SEQUENCE LISTING (210 sequences)

SEQ ID NO: 1 - DNA, 20 bp, Homo sapiens
atgatctctgctgccagtag

SEQ ID NO: 2 - DNA, 20 bp, Homo sapiens
atcgcgcctggatgtcaacg

SEQ ID NO: 3 - DNA, 20 bp, Homo sapiens
attgtgcagtgcatcggatt

SEQ ID NO: 4 - DNA, 20 bp, Homo sapiens
gtcaaacagtactgctacgg

SEQ ID NO: 5 - protein, 209 aa, Artificial Sequence
  Description of Artificial Sequence Synthetic polypeptide
LEPGEKPYKCPECGKSFSRSDKLTEHQRTHTGEKPYKCPECGKSFSTKNSLTEHQRTHTGEKPYKCPECGKSFSQSGDLRRHQRTHTGEKPYKCPECGKSFSTTGALTEHQRTHTGEKPYKCPECGKSFSDSGNLRVHQRTHTGEKPYKCPECGKSFSQRAHLERHQRTHTGEKPYKCPECGKSFSTSGELVRHQRTHTGEKPTGKKTS

SEQ ID NO: 6 - protein, 209 aa, Artificial Sequence
  Description of Artificial Sequence Synthetic polypeptide
LEPGEKPYKCPECGKSFSSPADLTRHQRTHTGEKPYKCPECGKSFSDPGHLVRHQRTHTGEKPYKCPECGKSFSRSDNLVRHQRTHTGEKPYKCPECGKSFSQSSSLVRHQRTHTGEKPYKCPECGKSFSTSHSLTEHQRTHTGEKPYKCPECGKSFSRNDALTEHQRTHTGEKPYKCPECGKSFSRNDALTEHQRTHTGEKPTGKKTS

SEQ ID NO: 7 - protein, 209 aa, Artificial Sequence
  Description of Artificial Sequence Synthetic polypeptide
LEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTS

SEQ ID NO: 8 - protein, 209 aa, Artificial Sequence
  Description of Artificial Sequence Synthetic polypeptide
LEPGEKPYKCPECGKSFSHTGHLLEHQRTHTGEKPYKCPECGKSFSTTGNLTVHQRTHTGEKPYKCPECGKSFSDKKDLTRHQRTHTGEKPYKCPECGKSFSQSGDLRRHQRTHTGEKPYKCPECGKSFSQRAHLERHQRTHTGEKPYKCPECGKSFSRSDKLTEHQRTHTGEKPYKCPECGKSFSRTDTLRDHQRTHTGEKPTGKKTS

SEQ ID NO: 9 - protein, 209 aa, Artificial Sequence
  Description of Artificial Sequence Synthetic polypeptide
LEPGEKPYKCPECGKSFSHTGHLLEHQRTHTGEKPYKCPECGKSFSTSGNLTEHQRTHTGEKPYKCPECGKSFSTSGNLVRHQRTHTGEKPYKCPECGKSFSQLAHLRAHQRTHTGEKPYKCPECGKSFSERSHLREHQRTHTGEKPYKCPECGKSFSDPGHLVRHQRTHTGEKPYKCPECGKSFSTHLDLIRHQRTHTGEKPTGKKTS

SEQ ID NO: 10 - protein, 209 aa, Artificial Sequence
  Description of Artificial Sequence Synthetic polypeptide
LEPGEKPYKCPECGKSFSDPGALVRHQRTHTGEKPYKCPECGKSFSERSHLREHQRTHTGEKPYKCPECGKSFSRSDHLTNHQRTHTGEKPYKCPECGKSFSERSHLREHQRTHTGEKPYKCPECGKSFSRSDHLTNHQRTHTGEKPYKCPECGKSFSDPGHLVRHQRTHTGEKPYKCPECGKSFSRSDKLVRHQRTHTGEKPTGKKTS

SEQ ID NO: 11 - protein, 209 aa, Artificial Sequence
  Description of Artificial Sequence Synthetic polypeptide
LEPGEKPYKCPECGKSFSRNDALTEHQRTHTGEKPYKCPECGKSFSDCRDLARHQRTHTGEKPYKCPECGKSFSDPGHLVRHQRTHTGEKPYKCPECGKSFSQSGHLTEHQRTHTGEKPYKCPECGKSFSREDNLHTHQRTHTGEKPYKCPECGKSFSTKNSLTEHQRTHTGEKPYKCPECGKSFSRADNLTEHQRTHTGEKPTGKKTS

SEQ ID NO: 12 - protein, 209 aa, Artificial Sequence
  Description of Artificial Sequence Synthetic polypeptide
LEPGEKPYKCPECGKSFSRSDKLTEHQRTHTGEKPYKCPECGKSFSRRDELNVHQRTHTGEKPYKCPECGKSFSRSDHLTNHQRTHTGEKPYKCPECGKSFSSPADLTRHQRTHTGEKPYKCPECGKSFSRSDHLTNHQRTHTGEKPYKCPECGKSFSSKKALTEHQRTHTGEKPYKCPECGKSFSTHLDLIRHQRTHTGEKPTGKKTS

SEQ ID NO: 13 - protein, 209 aa, Artificial Sequence
  Description of Artificial Sequence Synthetic polypeptide
LEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTS

SEQ ID NO: 14 - protein, 209 aa, Artificial Sequence
  Description of Artificial Sequence Synthetic polypeptide
LEPGEKPYKCPECGKSFSQSGDLRRHQRTHTGEKPYKCPECGKSFSQSGHLTEHQRTHTGEKPYKCPECGKSFSERSHLREHQRTHTGEKPYKCPECGKSFSDPGHLVRHQRTHTGEKPYKCPECGKSFSRNDTLTEHQRTHTGEKPYKCPECGKSFSRADNLTEHQRTHTGEKPYKCPECGKSFSTHLDLIRHQRTHTGEKPTGKKTS

SEQ ID NO: 15 - protein, 209 aa, Artificial Sequence
  Description of Artificial Sequence Synthetic polypeptide
LEPGEKPYKCPECGKSFSHTGHLLEHQRTHTGEKPYKCPECGKSFSSKKALTEHQRTHTGEKPYKCPECGKSFSDCRDLARHQRTHTGEKPYKCPECGKSFSHTGHLLEHQRTHTGEKPYKCPECGKSFSRNDALTEHQRTHTGEKPYKCPECGKSFSQSGDLRRHQRTHTGEKPYKCPECGKSFSDSGNLRVHQRTHTGEKPTGKKTS

SEQ ID NO: 16 - protein, 209 aa, Artificial Sequence
  Description of Artificial Sequence Synthetic polypeptide
LEPGEKPYKCPECGKSFSQSSSLVRHQRTHTGEKPYKCPECGKSFSRSDHLTNHQRTHTGEKPYKCPECGKSFSQLAHLRAHQRTHTGEKPYKCPECGKSFSQSSNLVRHQRTHTGEKPYKCPECGKSFSRSDNLVRHQRTHTGEKPYKCPECGKSFSRSDELVRHQRTHTGEKPYKCPECGKSFSQLAHLRAHQRTHTGEKPTGKKTS

SEQ ID NO: 17 - protein, 1367 aa, Artificial Sequence
  Description of Artificial Sequence Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 18 - protein, 96 aa, Artificial Sequence
  Description of Artificial Sequence Synthetic polypeptide
DAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 19 - protein, 385 aa, Artificial Sequence
  Description of Artificial Sequence Synthetic polypeptide
SKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 20 - protein, 480 aa, Artificial Sequence
  Description of Artificial Sequence Synthetic polypeptide
VDLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 21 - protein, 457 aa, Artificial Sequence
  Description of Artificial Sequence Synthetic polypeptide
EWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 22 - protein, 367 aa, Artificial Sequence
  Description of Artificial Sequence Synthetic polypeptide
MAPKKKRKVGIHGVPAAGSSGSLEPGEKPYKCPECGKSFSRSDKLTEHQRTHTGEKPYKCPECGKSFSTKNSLTEHQRTHTGEKPYKCPECGKSFSQSGDLRRHQRTHTGEKPYKCPECGKSFSTTGALTEHQRTHTGEKPYKCPECGKSFSDSGNLRVHQRTHTGEKPYKCPECGKSFSQRAHLERHQRTHTGEKPYKCPECGKSFSTSGELVRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 23 - protein, 367 aa, Artificial Sequence
  Description of Artificial Sequence Synthetic polypeptide
MAPKKKRKVGIHGVPAAGSSGSLEPGEKPYKCPECGKSFSSPADLTRHQRTHTGEKPYKCPECGKSFSDPGHLVRHQRTHTGEKPYKCPECGKSFSRSDNLVRHQRTHTGEKPYKCPECGKSFSQSSSLVRHQRTHTGEKPYKCPECGKSFSTSHSLTEHQRTHTGEKPYKCPECGKSFSRNDALTEHQRTHTGEKPYKCPECGKSFSRNDALTEHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 24 - protein, 367 aa, Artificial Sequence
  Description of Artificial Sequence Synthetic polypeptide
MAPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 25 - protein, 367 aa, Artificial Sequence
  Description of Artificial Sequence Synthetic polypeptide
MAPKKKRKVGIHGVPAAGSSGSLEPGEKPYKCPECGKSFSHTGHLLEHQRTHTGEKPYKCPECGKSFSTTGNLTVHQRTHTGEKPYKCPECGKSFSDKKDLTRHQRTHTGEKPYKCPECGKSFSQSGDLRRHQRTHTGEKPYKCPECGKSFSQRAHLERHQRTHTGEKPYKCPECGKSFSRSDKLTEHQRTHTGEKPYKCPECGKSFSRTDTLRDHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 26 - protein, 367 aa, Artificial Sequence
  Description of Artificial Sequence Synthetic polypeptide
MAPKKKRKVGIHGVPAAGSSGSLEPGEKPYKCPECGKSFSHTGHLLEHQRTHTGEKPYKCPECGKSFSTSGNLTEHQRTHTGEKPYKCPECGKSFSTSGNLVRHQRTHTGEKPYKCPECGKSFSQLAHLRAHQRTHTGEKPYKCPECGKSFSERSHLREHQRTHTGEKPYKCPECGKSFSDPGHLVRHQRTHTGEKPYKCPECGKSFSTHLDLIRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 27 - protein, 367 aa, Artificial Sequence
  Description of Artificial Sequence Synthetic polypeptide
MAPKKKRKVGIHGVPAAGSSGSLEPGEKPYKCPECGKSFSDPGALVRHQRTHTGEKPYKCPECGKSFSERSHLREHQRTHTGEKPYKCPECGKSFSRSDHLTNHQRTHTGEKPYKCPECGKSFSERSHLREHQRTHTGEKPYKCPECGKSFSRSDHLTNHQRTHTGEKPYKCPECGKSFSDPGHLVRHQRTHTGEKPYKCPECGKSFSRSDKLVRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 28 - protein, 661 aa, Artificial Sequence
  Description of Artificial Sequence Synthetic polypeptide
MAPKKKRKVGIHGVPAAGSSGSLEPGEKPYKCPECGKSFSRNDALTEHQRTHTGEKPYKCPECGKSFSDCRDLARHQRTHTGEKPYKCPECGKSFSDPGHLVRHQRTHTGEKPYKCPECGKSFSQSGHLTEHQRTHTGEKPYKCPECGKSFSREDNLHTHQRTHTGEKPYKCPECGKSFSTKNSLTEHQRTHTGEKPYKCPECGKSFSRADNLTEHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 29 - protein, 661 aa, Artificial Sequence
  Description of Artificial Sequence Synthetic polypeptide
MAPKKKRKVGIHGVPAAGSSGSLEPGEKPYKCPECGKSFSRSDKLTEHQRTHTGEKPYKCPECGKSFSRRDELNVHQRTHTGEKPYKCPECGKSFSRSDHLTNHQRTHTGEKPYKCPECGKSFSSPADLTRHQRTHTGEKPYKCPECGKSFSRSDHLTNHQRTHTGEKPYKCPECGKSFSSKKALTEHQRTHTGEKPYKCPECGKSFSTHLDLIRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 30 - protein, 661 aa, Artificial Sequence
  Description of Artificial Sequence Synthetic polypeptide
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 31 - protein, 661 aa, Artificial Sequence
  Description of Artificial Sequence Synthetic polypeptide
MAPKKKRKVGIHGVPAAGSSGSLEPGEKPYKCPECGKSFSQSGDLRRHQRTHTGEKPYKCPECGKSFSQSGHLTEHQRTHTGEKPYKCPECGKSFSERSHLREHQRTHTGEKPYKCPECGKSFSDPGHLVRHQRTHTGEKPYKCPECGKSFSRNDTLTEHQRTHTGEKPYKCPECGKSFSRADNLTEHQRTHTGEKPYKCPECGKSFSTHLDLIRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 32 - protein, 661 aa, Artificial Sequence
  Description of Artificial Sequence Synthetic polypeptide
MAPKKKRKVGIHGVPAAGSSGSLEPGEKPYKCPECGKSFSHTGHLLEHQRTHTGEKPYKCPECGKSFSSKKALTEHQRTHTGEKPYKCPECGKSFSDCRDLARHQRTHTGEKPYKCPECGKSFSHTGHLLEHQRTHTGEKPYKCPECGKSFSRNDALTEHQRTHTGEKPYKCPECGKSFSQSGDLRRHQRTHTGEKPYKCPECGKSFSDSGNLRVHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 33 - protein, 661 aa, Artificial Sequence
  Description of Artificial Sequence Synthetic polypeptide
MAPKKKRKVGIHGVPAAGSSGSLEPGEKPYKCPECGKSFSQSSSLVRHQRTHTGEKPYKCPECGKSFSRSDHLTNHQRTHTGEKPYKCPECGKSFSQLAHLRAHQRTHTGEKPYKCPECGKSFSQSSNLVRHQRTHTGEKPYKCPECGKSFSRSDNLVRHQRTHTGEKPYKCPECGKSFSRSDELVRHQRTHTGEKPYKCPECGKSFSQLAHLRAHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 34 - protein, 1528 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 35 - protein, 1820 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 36 - protein, 1922 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSVDLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKDGGGGSGKRPAATKKAGQAKKKKGSYPYDVPDYA

SEQ ID NO: 37 - protein, 1986 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSGGGGSGPKKKRKVAAAGSNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLSGGKRPAATKKAGQAKKKKGSYPYDVPDYA

SEQ ID NO: 38 - DNA, 627 bp, Artificial Sequence
  Description of Artificial Sequence Synthetic polynucleotide
ctggagcccggcgagaaaccctacaagtgccccgagtgcggcaaatccttctctagaagcgacaaactgaccgaacatcagaggacccacaccggcgagaagccttataagtgtcccgaatgcggcaaatccttcagcaccaagaactctctgacagaacaccagagaacacataccggagagaaaccttataaatgccccgagtgcggcaagtccttctcccagtccggcgatctgaggagacaccaaagaacacataccggcgaaaagccttacaagtgccccgagtgtggaaagagcttctccaccaccggcgctctgaccgagcaccagagaacacacaccggcgagaaaccctataaatgtcccgagtgtggcaaatccttcagcgacagcggcaatctgagagtgcaccaaagaacccataccggcgaaaaaccctacaaatgccccgagtgcggcaaatccttcagccagagggcccatctggagaggcaccaaaggacacacaccggagaaaagccctacaagtgtcccgagtgtggaaaaagctttagcacaagcggcgagctggtgaggcatcaaaggacccacaccggcgaaaagcccaccggcaaaaagaccagc

SEQ ID NO: 39 - DNA, 627 bp, Artificial Sequence
  Description of Artificial Sequence Synthetic polynucleotide
ctggagcccggcgagaagccctacaagtgccccgagtgcggaaagtccttcagctcccccgccgatctgacaagacatcagagaacccataccggcgagaaaccttacaaatgccccgaatgtggcaagtcctttagcgatcccggacatctggtgaggcaccagaggacacacaccggcgaaaagccctataaatgtcccgagtgtggaaagagcttttctagaagcgacaatctcgtgagacaccagagaacccacaccggagagaagccttacaagtgccccgagtgcggcaaatccttcagccagagctcctctctggtgaggcaccaaaggacccacaccggcgagaaaccttataagtgtcccgagtgtggcaaaagcttcagcacctcccactctctgaccgagcatcaaagaacccacaccggcgaaaaaccttataaatgccccgagtgtggcaaatccttcagcagaaatgacgctctgacagagcaccaaagaacacataccggagaaaagccctacaaatgccccgagtgtggaaaatccttttctagaaacgatgctctgaccgaacaccaaagaacacacaccggcgaaaagcctaccggaaaaaagaccagc

SEQ ID NO: 40 - DNA, 627 bp, Artificial Sequence
  Description of Artificial Sequence Synthetic polynucleotide
ctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagc

SEQ ID NO: 41 - DNA, 627 bp, Artificial Sequence
  Description of Artificial Sequence Synthetic polynucleotide
ctggagcccggcgaaaagccttataaatgtcccgaatgcggcaagagctttagccacaccggccatctgctggaacaccaaaggacccataccggcgaaaagccctataagtgccccgagtgtggcaagagcttcagcaccaccggcaatctgacagtccatcagaggacccacaccggagagaaaccctataaatgccccgagtgtggaaagtccttctccgacaagaaggatctgacaagacaccagaggacccataccggcgagaaaccctacaaatgccccgagtgcggcaaatccttctcccagagcggcgatctgaggagacatcaaagaacacataccggcgaaaaaccctataagtgccccgaatgcggcaagtccttcagccagagggcccatctggaaaggcatcagaggacacacaccggcgagaagccttacaaatgtcccgagtgcggaaagagcttctctagaagcgacaagctgaccgagcatcagaggacccacaccggagaaaaaccttacaagtgccccgagtgcggcaaaagcttcagcagaaccgacacactgagagatcaccaaaggacacacaccggcgagaaacccaccggcaaaaagaccagc

SEQ ID NO: 42 - DNA, 627 bp, Artificial Sequence
  Description of Artificial Sequence Synthetic polynucleotide
ctggagcccggcgagaagccttataagtgccccgagtgtggcaagagctttagccacaccggccatctgctggagcatcaaaggacacacaccggagaaaagccctataagtgccccgagtgtggcaaatccttcagcacctccggcaatctcaccgaacaccagagaacacacaccggagaaaaaccttacaaatgtcccgagtgtggaaagagcttttccaccagcggcaatctggtgagacatcaaagaacacataccggcgaaaaaccctataaatgccccgagtgtggaaaatccttctcccaactggcccatctgagggcccaccagaggacacataccggagaaaaaccctacaaatgccccgaatgcggaaaaagcttctccgagagaagccatctgagagagcaccaaaggacccataccggagaaaagccttacaagtgtcccgagtgcggaaaaagctttagcgatcccggacatctggtgagacatcagagaacccacaccggcgaaaagccttataaatgtcccgaatgtggcaagtcctttagcacccatctggatctgattagacaccaaagaacccacaccggcgagaaacccaccggaaaaaagaccagc

SEQ ID NO: 43 - DNA, 627 bp, Artificial Sequence
  Description of Artificial Sequence Synthetic polynucleotide
ctggagcccggcgaaaagccttacaaatgtcccgagtgcggaaagtccttcagcgaccccggcgctctggtgagacatcaaagaacacataccggcgagaaaccttataaatgccccgaatgtggaaaatccttcagcgaaagaagccatctgagggaacaccagaggacccacaccggcgaaaaaccttataagtgccccgaatgcggaaaaagcttttctagaagcgatcatctgaccaaccatcagagaacacacaccggcgaaaagccctataaatgtcccgagtgtggcaaatcctttagcgagaggtcccatctgagagagcaccagaggacacataccggagagaagccctacaagtgccccgagtgtggcaagagctttagcagaagcgaccatctgaccaatcatcaaaggacccacaccggagagaagccttacaagtgtcccgagtgcggaaagtccttttccgatcccggccacctcgtgaggcaccaaagaacccataccggcgagaaaccctacaaatgccccgagtgtggaaagagcttctccagaagcgacaagctggtgaggcatcagaggacacacaccggcgaaaaacccaccggcaagaaaaccagc

SEQ ID NO: 44 - DNA, 627 bp, Artificial Sequence
  Description of Artificial Sequence Synthetic polynucleotide
ctggagcccggagagaagccctacaaatgccccgagtgtggaaagagcttctctagaaatgacgctctgacagaacaccagaggacccataccggcgagaaaccttacaaatgccccgagtgcggaaaaagctttagcgattgcagagatctggctagacatcagagaacacacaccggcgagaagccctataagtgccccgaatgcggcaagagctttagcgaccccggccatctggtgagacatcaaaggacacataccggagaaaaaccttacaagtgccccgagtgcggaaagtccttctcccagagcggccatctcaccgagcatcaaaggacccacaccggcgaaaagccttataaatgtcccgaatgtggcaagtccttctctagagaggataatctgcacacccatcagaggacccacaccggcgaaaagccttataaatgccccgaatgtggaaagtccttttccaccaagaactctctgaccgagcatcagaggacacacaccggagagaaaccctataaatgtcccgagtgtggcaagagcttcagcagagccgacaatctgacagagcaccaaagaacacataccggcgaaaagcccaccggcaaaaagaccagc

SEQ ID NO: 45 - DNA, 627 bp, Artificial Sequence
  Description of Artificial Sequence Synthetic polynucleotide
ctggagcccggcgagaaaccctacaagtgccccgagtgtggcaaatccttctctagatccgacaaactgaccgaacatcagaggacccataccggcgaaaaaccttataaatgtcccgagtgcggaaagtccttctctagaagggacgagctgaacgtgcatcagagaacacataccggcgagaagccctataaatgccccgaatgcggcaaaagcttctctagaagcgatcatctgaccaaccaccagagaacccataccggagaaaagccttacaagtgtcccgaatgtggaaaatccttcagctcccccgccgatctgaccagacaccaaaggacccacaccggcgagaagccctataaatgccccgagtgcggcaagagcttttccagatccgaccatctgaccaatcatcaaagaacccacaccggcgaaaagccttataaatgtcccgagtgcggcaaatccttttccagcaagaaggctctgaccgagcatcaaaggacccataccggcgagaagccttacaaatgccccgagtgtggaaagtcctttagcacccatctggatctgattagacaccagaggacacacaccggagagaaacccaccggcaaaaagaccagc

SEQ ID NO: 46 - DNA, 627 bp, Artificial Sequence
  Description of Artificial Sequence Synthetic polynucleotide
ctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagc

SEQ ID NO: 47 - DNA, 627 bp, Artificial Sequence
  Description of Artificial Sequence Synthetic polynucleotide
ctggagcccggcgagaaaccctacaagtgccccgagtgtggaaaaagctttagccaaagcggcgatctgaggagacaccaaagaacacacaccggcgagaagccctacaaatgtcccgagtgcggaaagagcttcagccagagcggccatctgaccgagcatcagagaacccataccggcgaaaaaccttataagtgccccgagtgtggaaagtccttctccgagagatcccatctgagagaacaccagaggacacacaccggcgaaaaaccttataagtgtcccgagtgcggaaagtccttcagcgatcccggccatctggtgagacatcaaaggacacataccggcgaaaaaccttataagtgtcccgaatgcggcaagagctttagcagaaacgacacactcaccgaacaccagaggacccacaccggcgagaaaccctacaaatgccccgagtgcggcaaatccttttctagagccgacaatctgaccgaacaccagaggacccataccggagaaaagccttacaaatgtcccgagtgtggcaaatccttctccacccatctggatctgattagacaccaaagaacacataccggagaaaagcccaccggaaaaaagaccagc

SEQ ID NO: 48 - DNA, 627 bp, Artificial Sequence
  Description of Artificial Sequence Synthetic polynucleotide
ctggagcccggcgaaaaaccctataagtgccccgaatgtggaaagagcttcagccataccggccatctgctggaacaccaaaggacacacaccggcgagaaaccttacaagtgtcccgagtgcggaaaaagcttctcctccaaaaaggctctcaccgagcaccagagaacacataccggcgaaaagccttataagtgccccgagtgtggcaaatccttttccgactgtagagatctggccagacatcaaagaacccacaccggagagaaaccttataaatgccccgagtgcggcaagtcctttagccataccggccatctgctggagcaccagaggacccataccggcgagaagccttacaaatgccccgagtgcggcaaaagcttcagcagaaatgacgctctgaccgagcatcaaaggacccataccggcgaaaagccctacaagtgtcccgagtgtggaaagtccttctcccagagcggcgatctgaggagacaccagagaacacacaccggcgagaaaccctataaatgtcccgagtgcggaaagagctttagcgacagcggcaatctgagggtgcatcaaagaacacacaccggcgaaaaacccaccggaaaaaagacaagc

SEQ ID NO: 49 - DNA, 538 bp, Artificial Sequence
  Description of Artificial Sequence Synthetic polynucleotide
caccggcgaaaagccttataagtgccccgagtgcggcaagtccttctctagaagcgatcacctcaccaatcatcagaggacacataccggagagaagccctataagtgccccgagtgcggcaagagctttagccagctggctcatctgagagctcaccaaagaacccataccggcgagaagccttacaaatgccccgagtgtggaaaatccttttcccagtccagcaacctcgtcagacatcaaaggacccataccggcgaaaagccttacaagtgtcccgagtgcggaaagtccttctctagatccgacaacctcgtgaggcaccagagaacccacaccggcgagaaaccttacaaatgtcccgagtgtggcaaaagcttttctagaagcgacgagctggtgagacatcaaagaacccataccggcgaaaaaccttataagtgtcccgagtgcggcaaatcctttagccagctggcccatctgagggcccaccagagaacacataccggcgaaaaacccaccggcaaaaagacaagc

SEQ ID NO: 50 - DNA, 4101 bp, Artificial Sequence
  Description of Artificial Sequence Synthetic polynucleotide
gacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgac

SEQ ID NO: 51 - DNA, 288 bp, Artificial Sequence
  Description of Artificial Sequence Synthetic polynucleotide
gacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtg

SEQ ID NO: 52 - DNA, 1155 bp, Artificial Sequence
  Description of Artificial Sequence Synthetic polynucleotide
agcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggc

SEQ ID NO: 53 - DNA, 1440 bp, Artificial Sequence
  Description of Artificial Sequence Synthetic polynucleotide
gtggatctgaggacactcgacgtgtttagcggatgcggcggactctccgaaggcttccaccaagccggaatttccgacacactctgggccattgagatgtgggaccccgccgctcaagccttcagactgaataatcccggctccaccgtgttcaccgaggactgcaacattctgctgaagctggtgatggctggcgaaaccaccaactctagaggccagaggctgccccagaagggagatgtggaaatgctctgtggaggccctccttgccaaggcttctccggcatgaacaggttcaactctagaacatacagcaagttcaagaactctctggtcgtgagctttctgagctactgcgactactatagacctaggttctttctgctggagaacgtgagaaatttcgtgtccttcaagaggagcatggtgctgaagctgacactgaggtgtctggtgaggatgggctaccagtgcacattcggagtgctgcaagctggccagtacggcgtggcccagaccagaaggagggccatcattctggctgctgcccccggcgagaaactccctctgttccccgagcccctccacgtgttcgcccctagagcttgccagctgagcgtggtggtcgacgataagaagttcgtgagcaacatcacaaggctgtccagcggacccttcagaaccattaccgtgagggataccatgtccgacctccccgaggtgaggaatggcgccagcgctctggagatttcctacaacggcgaacctcagagctggttccaaaggcagctgagaggcgctcagtatcagcccattctgagggaccacatctgcaaagatatgagcgctctggtggccgctagaatgagacatattcctctggcccccggcagcgactggagagatctgcccaatattgaggtgagactcagcgacggaacaatggctagaaaactgaggtacacccatcatgatagaaagaacggaaggagcagcagcggcgctctgagaggagtgtgtagctgcgtggaagctggcaaggcttgcgatcccgccgctaggcagttcaataccctcatcccttggtgtctgcctcacaccggcaacagacacaatcattgggctggactgtatggaaggctcgaatgggacggctttttcagcaccaccgtgaccaatcccgaacctatgggcaagcaaggaagggtgctccaccccgagcagcatagagtcgtgtccgtgagagaatgcgctagaagccaaggcttccccgacacctatagactgttcggcaacattctggataagcacagacaagtgggaaatgctgtccctcctcctctggccaaggctatcggactggagatcaagctgtgtatgctcgccaaagctagggagagcgcttccgccaagattaaggaggaggaggccgccaaggac

SEQ ID NO: 54 - DNA, 1626 bp, Artificial Sequence
  Description of Artificial Sequence Synthetic polynucleotide
aaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctg

SEQ ID NO: 55 - DNA, 1345 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgagaaaccctacaagtgccccgagtgcggcaaatccttctctagaagcgacaaactgaccgaacatcagaggacccacaccggcgagaagccttataagtgtcccgaatgcggcaaatccttcagcaccaagaactctctgacagaacaccagagaacacataccggagagaaaccttataaatgccccgagtgcggcaagtccttctcccagtccggcgatctgaggagacaccaaagaacacataccggcgaaaagccttacaagtgccccgagtgtggaaagagcttctccaccaccggcgctctgaccgagcaccagagaacacacaccggcgagaaaccctataaatgtcccgagtgtggcaaatccttcagcgacagcggcaatctgagagtgcaccaaagaacccataccggcgaaaaaccctacaaatgccccgagtgcggcaaatccttcagccagagggcccatctggagaggcaccaaaggacacacaccggagaaaagccctacaagtgtcccgagtgtggaaaaagctttagcacaagcggcgagctggtgaggcatcaaaggacccacaccggcgaaaagcccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 56 - DNA, 1345 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgagaagccctacaagtgccccgagtgcggaaagtccttcagctcccccgccgatctgacaagacatcagagaacccataccggcgagaaaccttacaaatgccccgaatgtggcaagtcctttagcgatcccggacatctggtgaggcaccagaggacacacaccggcgaaaagccctataaatgtcccgagtgtggaaagagcttttctagaagcgacaatctcgtgagacaccagagaacccacaccggagagaagccttacaagtgccccgagtgcggcaaatccttcagccagagctcctctctggtgaggcaccaaaggacccacaccggcgagaaaccttataagtgtcccgagtgtggcaaaagcttcagcacctcccactctctgaccgagcatcaaagaacccacaccggcgaaaaaccttataaatgccccgagtgtggcaaatccttcagcagaaatgacgctctgacagagcaccaaagaacacataccggagaaaagccctacaaatgccccgagtgtggaaaatccttttctagaaacgatgctctgaccgaacaccaaagaacacacaccggcgaaaagcctaccggaaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 57 - DNA, 1345 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 58 - DNA, 1345 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgaaaagccttataaatgtcccgaatgcggcaagagctttagccacaccggccatctgctggaacaccaaaggacccataccggcgaaaagccctataagtgccccgagtgtggcaagagcttcagcaccaccggcaatctgacagtccatcagaggacccacaccggagagaaaccctataaatgccccgagtgtggaaagtccttctccgacaagaaggatctgacaagacaccagaggacccataccggcgagaaaccctacaaatgccccgagtgcggcaaatccttctcccagagcggcgatctgaggagacatcaaagaacacataccggcgaaaaaccctataagtgccccgaatgcggcaagtccttcagccagagggcccatctggaaaggcatcagaggacacacaccggcgagaagccttacaaatgtcccgagtgcggaaagagcttctctagaagcgacaagctgaccgagcatcagaggacccacaccggagaaaaaccttacaagtgccccgagtgcggcaaaagcttcagcagaaccgacacactgagagatcaccaaaggacacacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 59 - DNA, 1345 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgagaagccttataagtgccccgagtgtggcaagagctttagccacaccggccatctgctggagcatcaaaggacacacaccggagaaaagccctataagtgccccgagtgtggcaaatccttcagcacctccggcaatctcaccgaacaccagagaacacacaccggagaaaaaccttacaaatgtcccgagtgtggaaagagcttttccaccagcggcaatctggtgagacatcaaagaacacataccggcgaaaaaccctataaatgccccgagtgtggaaaatccttctcccaactggcccatctgagggcccaccagaggacacataccggagaaaaaccctacaaatgccccgaatgcggaaaaagcttctccgagagaagccatctgagagagcaccaaaggacccataccggagaaaagccttacaagtgtcccgagtgcggaaaaagctttagcgatcccggacatctggtgagacatcagagaacccacaccggcgaaaagccttataaatgtcccgaatgtggcaagtcctttagcacccatctggatctgattagacaccaaagaacccacaccggcgagaaacccaccggaaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 60 - DNA, 1345 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgaaaagccttacaaatgtcccgagtgcggaaagtccttcagcgaccccggcgctctggtgagacatcaaagaacacataccggcgagaaaccttataaatgccccgaatgtggaaaatccttcagcgaaagaagccatctgagggaacaccagaggacccacaccggcgaaaaaccttataagtgccccgaatgcggaaaaagcttttctagaagcgatcatctgaccaaccatcagagaacacacaccggcgaaaagccctataaatgtcccgagtgtggcaaatcctttagcgagaggtcccatctgagagagcaccagaggacacataccggagagaagccctacaagtgccccgagtgtggcaagagctttagcagaagcgaccatctgaccaatcatcaaaggacccacaccggagagaagccttacaagtgtcccgagtgcggaaagtccttttccgatcccggccacctcgtgaggcaccaaagaacccataccggcgagaaaccctacaaatgccccgagtgtggaaagagcttctccagaagcgacaagctggtgaggcatcagaggacacacaccggcgaaaaacccaccggcaagaaaaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 61 - DNA, 2227 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggagagaagccctacaaatgccccgagtgtggaaagagcttctctagaaatgacgctctgacagaacaccagaggacccataccggcgagaaaccttacaaatgccccgagtgcggaaaaagctttagcgattgcagagatctggctagacatcagagaacacacaccggcgagaagccctataagtgccccgaatgcggcaagagctttagcgaccccggccatctggtgagacatcaaaggacacataccggagaaaaaccttacaagtgccccgagtgcggaaagtccttctcccagagcggccatctcaccgagcatcaaaggacccacaccggcgaaaagccttataaatgtcccgaatgtggcaagtccttctctagagaggataatctgcacacccatcagaggacccacaccggcgaaaagccttataaatgccccgaatgtggaaagtccttttccaccaagaactctctgaccgagcatcagaggacacacaccggagagaaaccctataaatgtcccgagtgtggcaagagcttcagcagagccgacaatctgacagagcaccaaagaacacataccggcgaaaagcccaccggcaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - DNA, 2227 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgagaaaccctacaagtgccccgagtgtggcaaatccttctctagatccgacaaactgaccgaacatcagaggacccataccggcgaaaaaccttataaatgtcccgagtgcggaaagtccttctctagaagggacgagctgaacgtgcatcagagaacacataccggcgagaagccctataaatgccccgaatgcggcaaaagcttctctagaagcgatcatctgaccaaccaccagagaacccataccggagaaaagccttacaagtgtcccgaatgtggaaaatccttcagctcccccgccgatctgaccagacaccaaaggacccacaccggcgagaagccctataaatgccccgagtgcggcaagagcttttccagatccgaccatctgaccaatcatcaaagaacccacaccggcgaaaagccttataaatgtcccgagtgcggcaaatccttttccagcaagaaggctctgaccgagcatcaaaggacccataccggcgagaagccttacaaatgccccgagtgtggaaagtcctttagcacccatctggatctgattagacaccagaggacacacaccggagagaaacccaccggcaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 63 - DNA, 2227 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 64 - DNA, 2080 bp, Artificial Sequence
  Description of Artificial Sequence Synthetic polynucleotide
agtgtggaaaaagctttagccaaagcggcgatctgaggagacaccaaagaacacacaccggcgagaagccctacaaatgtcccgagtgcggaaagagcttcagccagagcggccatctgaccgagcatcagagaacccataccggcgaaaaaccttataagtgccccgagtgtggaaagtccttctccgagagatcccatctgagagaacaccagaggacacacaccggcgaaaaaccttataagtgtcccgagtgcggaaagtccttcagcgatcccggccatctggtgagacatcaaaggacacataccggcgaaaaaccttataagtgtcccgaatgcggcaagagctttagcagaaacgacacactcaccgaacaccagaggacccacaccggcgagaaaccctacaaatgccccgagtgcggcaaatccttttctagagccgacaatctgaccgaacaccagaggacccataccggagaaaagccttacaaatgtcccgagtgtggcaaatccttctccacccatctggatctgattagacaccaaagaacacataccggagaaaagcccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - DNA, 1929 bp, Artificial Sequence
  Description of Artificial Sequence Synthetic polynucleotide
gccttataagtgccccgagtgtggcaaatccttttccgactgtagagatctggccagacatcaaagaacccacaccggagagaaaccttataaatgccccgagtgcggcaagtcctttagccataccggccatctgctggagcaccagaggacccataccggcgagaagccttacaaatgccccgagtgcggcaaaagcttcagcagaaatgacgctctgaccgagcatcaaaggacccataccggcgaaaagccctacaagtgtcccgagtgtggaaagtccttctcccagagcggcgatctgaggagacaccagagaacacacaccggcgagaaaccctataaatgtcccgagtgcggaaagagctttagcgacagcggcaatctgagggtgcatcaaagaacacacaccggcgaaaaacccaccggaaaaaagacaagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - DNA, 1779 bp, Artificial Sequence
  Description of Artificial Sequence Synthetic polynucleotide
gacccataccggcgaaaagccttacaagtgtcccgagtgcggaaagtccttctctagatccgacaacctcgtgaggcaccagagaacccacaccggcgagaaaccttacaaatgtcccgagtgtggcaaaagcttttctagaagcgacgagctggtgagacatcaaagaacccataccggcgaaaaaccttataagtgtcccgagtgcggcaaatcctttagccagctggcccatctgagggcccaccagagaacacataccggcgaaaaacccaccggcaaaaagacaagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - DNA, 4829 bp, Artificial Sequence
  Description of Artificial Sequence Synthetic polynucleotide
aaggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaaggccagcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 68 - DNA, 2591 bp, Artificial Sequence
  Description of Artificial Sequence Synthetic polynucleotide
gagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaaggcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 69 - DNA, 6010 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagtcgggcgggggtggctcagtggatctgaggacactcgacgtgtttagcggatgcggcggactctccgaaggcttccaccaagccggaatttccgacacactctgggccattgagatgtgggaccccgccgctcaagccttcagactgaataatcccggctccaccgtgttcaccgaggactgcaacattctgctgaagctggtgatggctggcgaaaccaccaactctagaggccagaggctgccccagaagggagatgtggaaatgctctgtggaggccctccttgccaaggcttctccggcatgaacaggttcaactctagaacatacagcaagttcaagaactctctggtcgtgagctttctgagctactgcgactactatagacctaggttctttctgctggagaacgtgagaaatttcgtgtccttcaagaggagcatggtgctgaagctgacactgaggtgtctggtgaggatgggctaccagtgcacattcggagtgctgcaagctggccagtacggcgtggcccagaccagaaggagggccatcattctggctgctgcccccggcgagaaactccctctgttccccgagcccctccacgtgttcgcccctagagcttgccagctgagcgtggtggtcgacgataagaagttcgtgagcaacatcacaaggctgtccagcggacccttcagaaccattaccgtgagggataccatgtccgacctccccgaggtgaggaatggcgccagcgctctggagatttcctacaacggcgaacctcagagctggttccaaaggcagctgagaggcgctcagtatcagcccattctgagggaccacatctgcaaagatatgagcgctctggtggccgctagaatgagacatattcctctggcccccggcagcgactggagagatctgcccaatattgaggtgagactcagcgacggaacaatggctagaaaactgaggtacacccatcatgatagaaagaacggaaggagcagcagcggcgctctgagaggagtgtgtagctgcgtggaagctggcaaggcttgcgatcccgccgctaggcagttcaataccctcatcccttggtgtctgcctcacaccggcaacagacacaatcattgggctggactgtatggaaggctcgaatgggacggctttttcagcaccaccgtgaccaatcccgaacctatgggcaagcaaggaagggtgctccaccccgagcagcatagagtcgtgtccgtgagagaatgcgctagaagccaaggcttccccgacacctatagactgttcggcaacattctggataagcacagacaagtgggaaatgctgtccctcctcctctggccaaggctatcggactggagatcaagctgtgtatgctcgccaaagctagggagagcgcttccgccaagattaaggaggaggaggccgccaaggacggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 70 - DNA, 6202 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcgccggcggcggcggcagcggcggcggcggcagcggcggcggcggcagcggccccaagaagaagcggaaggtggccgccgccggcagcaaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 71 - DNA, 20 bp, Artificial Sequence
  Description of Artificial Sequence Synthetic oligonucleotide
  modified_base (1)..(1): a, c, t, g, unknown or other
  modified_base (3)..(3): a, c, t, g, unknown or other
nbndccdshagrkgghrshv

SEQ ID NO: 72 - DNA, 20 bp, Artificial Sequence
  Description of Artificial Sequence Synthetic oligonucleotide
  modified_base (18)..(18): a, c, t, g, unknown or other
  modified_base (20)..(20): a, c, t, g, unknown or other
vhsrhggkrgahsdccdnbn

SEQ ID NO: 73 - DNA, 10 bp, Artificial Sequence
  Description of Artificial Sequence Synthetic oligonucleotide
  modified_base (8)..(8): a, c, t, g, unknown or other
ccgccatntt

SEQ ID NO: 74 - DNA, 10 bp, Artificial Sequence
  Description of Artificial Sequence Synthetic oligonucleotide
  modified_base (3)..(3): a, c, t, g, unknown or other
aanatggcgg

SEQ ID NO: 75 - DNA, 21 bp, Homo sapiens
gctggaaaccttgcacctcgg

SEQ ID NO: 76 - DNA, 21 bp, Homo sapiens
ctgctgccagtagagggcaca

SEQ ID NO: 77 - DNA, 21 bp, Homo sapiens
gcccagagagggggcggaggg

SEQ ID NO: 78 - DNA, 21 bp, Homo sapiens
acgcggggagcaaccaatcgc

SEQ ID NO: 79 - DNA, 21 bp, Homo sapiens
actggcagcagagatcatcgc

SEQ ID NO: 80 - DNA, 21 bp, Homo sapiens
gggggcaggagcaggagcgtc

SEQ ID NO: 81 - DNA, 21 bp, Homo sapiens
cagccttagcgaggcgccctg

SEQ ID NO: 82 - DNA, 21 bp, Homo sapiens
actcacaggacaaggatgcgg

SEQ ID NO: 83 - DNA, 21 bp, Homo sapiens
agcaaaagaaaatggtaggcg

SEQ ID NO: 84 - DNA, 21 bp, Homo sapiens
actcagccgggcagccgagca

SEQ ID NO: 85 - DNA, 21 bp, Homo sapiens
cgtaccaggctgcagggcgcc

SEQ ID NO: 86 - DNA, 21 bp, Homo sapiens
agagtggaggaaagaagggta

SEQ ID NO: 87 - protein, 386 aa, Artificial Sequence
  Description of Artificial Sequence Synthetic polypeptide
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 88 - protein, 22 aa, Artificial Sequence
  Description of Artificial Sequence Synthetic peptide
MAPKKKRKVGIHGVPAAGSSGS

SEQ ID NO: 89 - protein, 29 aa, Artificial Sequence
  Description of Artificial Sequence Synthetic peptide
SGGKRPAATKKAGQAKKKGSYPYDVPDYA

SEQ ID NO: 90 - protein, 9 aa, Artificial Sequence
  Description of Artificial Sequence Synthetic peptide
YPYDVPDYA

SEQ ID NO: 91 - protein, 1123 aa, Artificial Sequence
  Description of Artificial Sequence Synthetic polypeptide
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYASSGGKRPAATKKAGQAKKKKGSYPYDVPDYA

SEQ ID NO: 92 - protein, 1123 aa, Artificial Sequence
  Description of Artificial Sequence Synthetic polypeptide
MAPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYASSGGKRPAATKKAGQAKKKKGSYPYDVPDYA

SEQ ID NO: 93 - DNA, 3332 bp, Artificial Sequence
  Description of Artificial Sequence Synthetic polynucleotide
gcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgccgggtcctacccgtacgacgtgcccgattacgccgccaccaacttctcgctgctgaagcaggccggagatgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgaagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 94 - DNA, 3012 bp, Artificial Sequence
  Description of Artificial Sequence Synthetic polynucleotide
tataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcagcggcggcaagcggcccgccgccaccaagaaggccggccaggccaagaagaagaagggcagctacccctacgacgtgcccgactacgccgggtcctacccgtacgacgtgcccgattacgccgccaccaacttctcgctgctgaagcaggccggagatgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcggcggcaagcggcccgccgccaccaagaaggccggccaggccaagaagaagaagggcagctacccctacgacgtgcccgactacgcctgaagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 95 - protein, 118 aa, Artificial Sequence
  Description of Artificial Sequence Synthetic polypeptide
PSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGS

SEQ ID NO: 96 - DNA, 20 bp, Homo sapiens
tgccacttccccactaaccc

SEQ ID NO: 97 - DNA, 20 bp, Homo sapiens
ggccacacaaggaagctgca

SEQ ID NO: 98 - DNA, 20 bp, Homo sapiens
ccacacaaggaagctgcagg

SEQ ID NO: 99 - DNA, 20 bp, Homo sapiens
tgattggaatgcaacccgaa

SEQ ID NO: 100 - DNA, 20 bp, Homo sapiens
ttttgcccttgctaccccaa

SEQ ID NO: 101 - DNA, 20 bp, Homo sapiens
agctgatggtatccactagg

SEQ ID NO: 102 - DNA, 20 bp, Homo sapiens
cacatccaagaatgtagtgg

SEQ ID NO: 103 - DNA, 20 bp, Homo sapiens
gatacagccacaaagctcac

SEQ ID NO: 104 - DNA, 20 bp, Homo sapiens
attacataacagaatccagg

SEQ ID NO: 105 - DNA, 20 bp, Homo sapiens
cccttgactgtgctgccacc

SEQ ID NO: 106 - DNA, 20 bp, Homo sapiens
cagacgaggaacctgaaccc

SEQ ID NO: 107 - DNA, 20 bp, Homo sapiens
agaatcccttggggtagcaa

SEQ ID NO: 108 - DNA, 20 bp, Homo sapiens
cagcactctcgctgaccgca

SEQ ID NO: 109 - DNA, 20 bp, Homo sapiens
gttgagtcatgtgtactctg

SEQ ID NO: 110 - DNA, 20 bp, Homo sapiens
aggaacaggatgttacaact

SEQ ID NO: 111 - DNA, 20 bp, Homo sapiens
ggggccactagggacaggat

SEQ ID NO: 112 - DNA, 3650 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgccgggtcctacccgtacgacgtgcccgattacgccgccaccaacttctcgctgctgaagcaggccggagatgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgaagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 113 - DNA, 3650 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcagcggcggcaagcggcccgccgccaccaagaaggccggccaggccaagaagaagaagggcagctacccctacgacgtgcccgactacgccgggtcctacccgtacgacgtgcccgattacgccgccaccaacttctcgctgctgaagcaggccggagatgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcggcggcaagcggcccgccgccaccaagaaggccggccaggccaagaagaagaagggcagctacccctacgacgtgcccgactacgcctgaagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 114 - protein, 542 aa, Artificial Sequence
  Description of Artificial Sequence Synthetic polypeptide
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 115 - DNA, 627 bp, Artificial Sequence
  Description of Artificial Sequence Synthetic polynucleotide
ctggagcccggcgagaaaccctataaatgccccgaatgcggaaaaagcttcagccagtccagctctctggtgagacatcagaggacacacaccggcgaaaagccttataagtgccccgagtgcggcaagtccttctctagaagcgatcacctcaccaatcatcagaggacacataccggagagaagccctataagtgccccgagtgcggcaagagctttagccagctggctcatctgagagctcaccaaagaacccataccggcgagaagccttacaaatgccccgagtgtggaaaatccttttcccagtccagcaacctcgtcagacatcaaaggacccataccggcgaaaagccttacaagtgtcccgagtgcggaaagtccttctctagatccgacaacctcgtgaggcaccagagaacccacaccggcgagaaaccttacaaatgtcccgagtgtggcaaaagcttttctagaagcgacgagctggtgagacatcaaagaacccataccggcgaaaaaccttataagtgtcccgagtgcggcaaatcctttagccagctggcccatctgagggcccaccagagaacacataccggcgaaaaacccaccggcaaaaagacaagc

SEQ ID NO: 116 - DNA, 2227 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgagaaaccctacaagtgccccgagtgtggaaaaagctttagccaaagcggcgatctgaggagacaccaaagaacacacaccggcgagaagccctacaaatgtcccgagtgcggaaagagcttcagccagagcggccatctgaccgagcatcagagaacccataccggcgaaaaaccttataagtgccccgagtgtggaaagtccttctccgagagatcccatctgagagaacaccagaggacacacaccggcgaaaaaccttataagtgtcccgagtgcggaaagtccttcagcgatcccggccatctggtgagacatcaaaggacacataccggcgaaaaaccttataagtgtcccgaatgcggcaagagctttagcagaaacgacacactcaccgaacaccagaggacccacaccggcgagaaaccctacaaatgccccgagtgcggcaaatccttttctagagccgacaatctgaccgaacaccagaggacccataccggagaaaagccttacaaatgtcccgagtgtggcaaatccttctccacccatctggatctgattagacaccaaagaacacataccggagaaaagcccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 117 - DNA, 2227 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgaaaaaccctataagtgccccgaatgtggaaagagcttcagccataccggccatctgctggaacaccaaaggacacacaccggcgagaaaccttacaagtgtcccgagtgcggaaaaagcttctcctccaaaaaggctctcaccgagcaccagagaacacataccggcgaaaagccttataagtgccccgagtgtggcaaatccttttccgactgtagagatctggccagacatcaaagaacccacaccggagagaaaccttataaatgccccgagtgcggcaagtcctttagccataccggccatctgctggagcaccagaggacccataccggcgagaagccttacaaatgccccgagtgcggcaaaagcttcagcagaaatgacgctctgaccgagcatcaaaggacccataccggcgaaaagccctacaagtgtcccgagtgtggaaagtccttctcccagagcggcgatctgaggagacaccagagaacacacaccggcgagaaaccctataaatgtcccgagtgcggaaagagctttagcgacagcggcaatctgagggtgcatcaaagaacacacaccggcgaaaaacccaccggaaaaaagacaagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 118 - DNA, 2227 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcccggcgagaaaccctataaatgccccgaatgcggaaaaagcttcagccagtccagctctctggtgagacatcagaggacacacaccggcgaaaagccttataagtgccccgagtgcggcaagtccttctctagaagcgatcacctcaccaatcatcagaggacacataccggagagaagccctataagtgccccgagtgcggcaagagctttagccagctggctcatctgagagctcaccaaagaacccataccggcgagaagccttacaaatgccccgagtgtggaaaatccttttcccagtccagcaacctcgtcagacatcaaaggacccataccggcgaaaagccttacaagtgtcccgagtgcggaaagtccttctctagatccgacaacctcgtgaggcaccagagaacccacaccggcgagaaaccttacaaatgtcccgagtgtggcaaaagcttttctagaagcgacgagctggtgagacatcaaagaacccataccggcgaaaaaccttataagtgtcccgagtgcggcaaatcctttagccagctggcccatctgagggcccaccagagaacacataccggcgaaaaacccaccggcaaaaagacaagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 119 - DNA, 5705 bp, Artificial Sequence
  Description of Artificial Sequence Synthetic polynucleotide
aaggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaaggcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 120 - protein, 18 aa, Artificial Sequence
  Description of Artificial Sequence Synthetic peptide
EGRGSLLTCGDVEENPGP

SEQ ID NO: 121 - protein, 1092 aa, Artificial Sequence
  Description of Artificial Sequence Synthetic polypeptide
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 122 - protein, 1092 aa, Artificial Sequence
  Description of Artificial Sequence Synthetic polypeptide
MAPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYA

SEQ ID NO: 123 - DNA, 355 bp, Homo sapiens
cagagaaggaggaagttaattcacattcttaattttttctaagggcaaaaaaaaaaaaaaaatgcaccagctcattttccatctctgcttgggtcatcagtgtgcattgtgagcctgtacaaaggccttagacggggaatgctgccgagagcatcaccttttatgtcttcttttatatgaaatgtgccacttccccactaaccctggctctgggctctgcctctgctctcctgatggtgtgtttatggtggattcagcattctgggccacacaaggaagctgcagggggtgtccaagttcacatgtccccgcattccaggcgaatgtttctgacattgagcaatgatatggctct

SEQ ID NO: 124 - protein, 46 aa, Artificial Sequence
  Description of Artificial Sequence Synthetic polypeptide
ATNFSLLKQAGDVEENPGPTSAGKLGSGEGRGSLLTCGDVEENPGP

SEQ ID NO: 125 - protein, 19 aa, Artificial Sequence
  Description of Artificial Sequence Synthetic peptide
ATNFSLLKQAGDVEENPGP

SEQ ID NO: 126 - protein, 18 aa, Artificial Sequence
  Description of Artificial Sequence Synthetic peptide
ATNFSLLKQAGDVEENPG

SEQ ID NO: 127 - protein, 27 aa, Artificial Sequence
  Description of Artificial Sequence Synthetic peptide
PTSAGKLGSGEGRGSLLTCGDVEENPG

SEQ ID NO: 128 - protein, 1 aa, Artificial Sequence
  Description of Artificial Sequence Synthetic peptide
P

SEQ ID NO: 129 - protein, 652 aa, Artificial Sequence
  Description of Artificial Sequence Synthetic polypeptide
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 130 - DNA, 2234 bp, Artificial Sequence
  Description of Artificial Sequence Synthetic polynucleotide
gggaaataagagagaaaagaagagtaagaagaaatataagagccaccatggcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 131 - RNA, 627 nt, Artificial Sequence
  Description of Artificial Sequence Synthetic polynucleotide
cuggagcccggcgagaaaccuuacaaaugccccgagugcggcaagagcuucagcagaagcgacgaucuggugaggcaccaaagaacccacaccggcgaaaaaccuuacaagugucccgaaugcggaaaguccuucagcagagaggacaaucugcacacccaccagagaacacacaccggagaaaagccuuacaagugccccgaaugcggcaaauccuuuucuagaagcgaucaucugaccacccaccaaagaacacauaccggcgagaagccuuacaaaugucccgagugcggaaaguccuucucccagagagccaaucugagggcucaucaaaggacccauaccggcgaaaagcccuacaaaugccccgagugcggaaaauccuucagccagcuggcccaucugagagcccaccaaaggacacacaccggagagaaacccuauaagugccccgaguguggaaaaagcuuuucccagagggccaaucugagggcccaucagaggacccauaccggagagaagccuuauaaaugucccgagugcggaaaaagcuucagcgagaggagccaucugagggaacaucaaagaacccacaccggcgaaaaacccaccggaaaaaagaccagc

SEQ ID NO: 132 - RNA, 1155 nt, Artificial Sequence
  Description of Artificial Sequence Synthetic polynucleotide
agcaagguggagaacaagaccaagaagcugcggguguucgaggccuucgccggcaucggcgcccagcggaaggcccuggagaaggugcggaaggacgaguacgagaucgugggccuggccgagugguacgugcccgccaucgugauguaccaggccauccacaacaacuuccacaccaagcuggaguacaagagcgugagccgggaggagaugaucgacuaccuggagaacaagacccugagcuggaacagcaagaaccccgugagcaacggcuacuggaagcggaagaaggacgacgagcugaagaucaucuacaacgccaucaagcugagcgagaaggagggcaacaucuucgacauccgggaccuguacaagcggacccugaagaacaucgaccugcugaccuacagcuuccccugccaggaccugagccagcagggcauccagaagggcaugaagcggggcagcggcacccggagcggccugcugugggagaucgagcgggcccuggacagcaccgagaagaacgaccugcccaaguaccugcugauggagaacgugggcgcccugcugcacaagaagaacgaggaggagcugaaccaguggaagcagaagcuggagagccugggcuaccagaacagcaucgaggugcugaacgccgccgacuucggcagcagccaggcccggcggcggguguucaugaucagcacccugaacgaguucguggagcugcccaagggcgacaagaagcccaagagcaucaagaaggugcugaacaagaucgugagcgagaaggacauccugaacaaccugcugaaguacaaccugaccgaguucaagaaaaccaagagcaacaucaacaaggccagccugaucggcuacagcaaguucaacagcgagggcuacguguacgaccccgaguucaccggccccacccugaccgccagcggcgccaacagccggaucaagaucaaggacggcagcaacauccggaagaugaacagcgacgagaccuuccuguacaucggcuucgacagccaggacggcaagcgggugaacgagaucgaguuccugaccgagaaccagaagaucuucgugugcggcaacagcaucagcguggaggugcuggaggccaucaucgacaagaucggcggc

SEQ ID NO: 133 - protein, 690 aa, Artificial Sequence
  Description of Artificial Sequence Synthetic polypeptide
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYAGSYPYDVPDYAATNFSLLKQAGDVEENPG

SEQ ID NO: 134 - protein, 366 aa, Artificial Sequence
  Description of Artificial Sequence Synthetic polypeptide
PLEGSSGSGSPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 135 - protein, 6 aa, Artificial Sequence
  Description of Artificial Sequence Synthetic peptide
PKKKRK

SEQ ID NO: 136 - protein, 15 aa, Artificial Sequence
  Description of Artificial Sequence Synthetic peptide
KRPAATKKAGQAKKK

SEQ ID NO: 137 - protein, 13 aa, Artificial Sequence
  Description of Artificial Sequence Synthetic peptide
ASGSGGGSGGARD

SEQ ID NO: 138 - protein, 10 aa, Artificial Sequence
  Description of Artificial Sequence Synthetic peptide
ASGSGGGSGG

SEQ ID NO: 139 - protein, 358 aa, Artificial Sequence
  Description of Artificial Sequence Synthetic polypeptide
MAPKKKRKVGIHGVPAAGSSGSLEPGEKPYKCPECGKSFSRSDKLTEHQRTHTGEKPYKCPECGKSFSTKNSLTEHQRTHTGEKPYKCPECGKSFSQSGDLRRHQRTHTGEKPYKCPECGKSFSTTGALTEHQRTHTGEKPYKCPECGKSFSDSGNLRVHQRTHTGEKPYKCPECGKSFSQRAHLERHQRTHTGEKPYKCPECGKSFSTSGELVRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 140 - protein, 358 aa, Artificial Sequence
  Description of Artificial Sequence Synthetic polypeptide
MAPKKKRKVGIHGVPAAGSSGSLEPGEKPYKCPECGKSFSSPADLTRHQRTHTGEKPYKCPECGKSFSDPGHLVRHQRTHTGEKPYKCPECGKSFSRSDNLVRHQRTHTGEKPYKCPECGKSFSQSSSLVRHQRTHTGEKPYKCPECGKSFSTSHSLTEHQRTHTGEKPYKCPECGKSFSRNDALTEHQRTHTGEKPYKCPECGKSFSRNDALTEHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 141 - protein, 358 aa, Artificial Sequence
  Description of Artificial Sequence Synthetic polypeptide
MAPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 142 - protein, 358 aa, Artificial Sequence
  Description of Artificial Sequence Synthetic polypeptide
MAPKKKRKVGIHGVPAAGSSGSLEPGEKPYKCPECGKSFSHTGHLLEHQRTHTGEKPYKCPECGKSFSTTGNLTVHQRTHTGEKPYKCPECGKSFSDKKDLTRHQRTHTGEKPYKCPECGKSFSQSGDLRRHQRTHTGEKPYKCPECGKSFSQRAHLERHQRTHTGEKPYKCPECGKSFSRSDKLTEHQRTHTGEKPYKCPECGKSFSRTDTLRDHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 143 - protein, 358 aa, Artificial Sequence
  Description of Artificial Sequence Synthetic polypeptide
MAPKKKRKVGIHGVPAAGSSGSLEPGEKPYKCPECGKSFSHTGHLLEHQRTHTGEKPYKCPECGKSFSTSGNLTEHQRTHTGEKPYKCPECGKSFSTSGNLVRHQRTHTGEKPYKCPECGKSFSQLAHLRAHQRTHTGEKPYKCPECGKSFSERSHLREHQRTHTGEKPYKCPECGKSFSDPGHLVRHQRTHTGEKPYKCPECGKSFSTHLDLIRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 144 - protein, 358 aa, Artificial Sequence
  Description of Artificial Sequence Synthetic polypeptide
MAPKKKRKVGIHGVPAAGSSGSLEPGEKPYKCPECGKSFSDPGALVRHQRTHTGEKPYKCPECGKSFSERSHLREHQRTHTGEKPYKCPECGKSFSRSDHLTNHQRTHTGEKPYKCPECGKSFSERSHLREHQRTHTGEKPYKCPECGKSFSRSDHLTNHQRTHTGEKPYKCPECGKSFSDPGHLVRHQRTHTGEKPYKCPECGKSFSRSDKLVRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 145 - protein, 652 aa, Artificial Sequence
  Description of Artificial Sequence Synthetic polypeptide
MAPKKKRKVGIHGVPAAGSSGSLEPGEKPYKCPECGKSFSRNDALTEHQRTHTGEKPYKCPECGKSFSDCRDLARHQRTHTGEKPYKCPECGKSFSDPGHLVRHQRTHTGEKPYKCPECGKSFSQSGHLTEHQRTHTGEKPYKCPECGKSFSREDNLHTHQRTHTGEKPYKCPECGKSFSTKNSLTEHQRTHTGEKPYKCPECGKSFSRADNLTEHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 146 - protein, 652 aa, Artificial Sequence
  Description of Artificial Sequence Synthetic polypeptide
MAPKKKRKVGIHGVPAAGSSGSLEPGEKPYKCPECGKSFSRSDKLTEHQRTHTGEKPYKCPECGKSFSRRDELNVHQRTHTGEKPYKCPECGKSFSRSDHLTNHQRTHTGEKPYKCPECGKSFSSPADLTRHQRTHTGEKPYKCPECGKSFSRSDHLTNHQRTHTGEKPYKCPECGKSFSSKKALTEHQRTHTGEKPYKCPECGKSFSTHLDLIRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 147 - protein, 652 aa, Artificial Sequence
  Description of Artificial Sequence Synthetic polypeptide
MAPKKKRKVGIHGVPAAGSSGSLEPGEKPYKCPECGKSFSQSGDLRRHQRTHTGEKPYKCPECGKSFSQSGHLTEHQRTHTGEKPYKCPECGKSFSERSHLREHQRTHTGEKPYKCPECGKSFSDPGHLVRHQRTHTGEKPYKCPECGKSFSRNDTLTEHQRTHTGEKPYKCPECGKSFSRADNLTEHQRTHTGEKPYKCPECGKSFSTHLDLIRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 148 - protein, 652 aa, Artificial Sequence
  Description of Artificial Sequence Synthetic polypeptide
MAPKKKRKVGIHGVPAAGSSGSLEPGEKPYKCPECGKSFSHTGHLLEHQRTHTGEKPYKCPECGKSFSSKKALTEHQRTHTGEKPYKCPECGKSFSDCRDLARHQRTHTGEKPYKCPECGKSFSHTGHLLEHQRTHTGEKPYKCPECGKSFSRNDALTEHQRTHTGEKPYKCPECGKSFSQSGDLRRHQRTHTGEKPYKCPECGKSFSDSGNLRVHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 149 - protein, 652 aa, Artificial Sequence
  Description of Artificial Sequence Synthetic polypeptide
MAPKKKRKVGIHGVPAAGSSGSLEPGEKPYKCPECGKSFSQSSSLVRHQRTHTGEKPYKCPECGKSFSRSDHLTNHQRTHTGEKPYKCPECGKSFSQLAHLRAHQRTHTGEKPYKCPECGKSFSQSSNLVRHQRTHTGEKPYKCPECGKSFSRSDNLVRHQRTHTGEKPYKCPECGKSFSRSDELVRHQRTHTGEKPYKCPECGKSFSQLAHLRAHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 150 - protein, 1519 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 151 - protein, 1811 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 152 - protein, 1913 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSVDLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKDGGGGSGKRPAATKKAGQAKKKKGS

SEQ ID NO: 153 - protein, 1977 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSGGGGSGPKKKRKVAAAGSNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLSGGKRPAATKKAGQAKKKKGS

SEQ ID NO: 154 - protein, 209 aa, Artificial Sequence
  Description of Artificial Sequence Synthetic polypeptide
LEPGEKPYKCPECGKSFSTSGELVRHQRTHTGEKPYKCPECGKSFSERSHLREHQRTHTGEKPYKCPECGKSFSRNDTLTEHQRTHTGEKPYKCPECGKSFSRADNLTEHQRTHTGEKPYKCPECGKSFSTSGSLVRHQRTHTGEKPYKCPECGKSFSSPADLTRHQRTHTGEKPYKCPECGKSFSDSGNLRVHQRTHTGEKPTGKKTS

SEQ ID NO: 155 - protein, 209 aa, Artificial Sequence
  Description of Artificial Sequence Synthetic polypeptide
LEPGEKPYKCPECGKSFSRSDKLVRHQRTHTGEKPYKCPECGKSFSQRANLRAHQRTHTGEKPYKCPECGKSFSRADNLTEHQRTHTGEKPYKCPECGKSFSQSSSLVRHQRTHTGEKPYKCPECGKSFSDKKDLTRHQRTHTGEKPYKCPECGKSFSSPADLTRHQRTHTGEKPYKCPECGKSFSQSGHLTEHQRTHTGEKPTGKKTS

SEQ ID NO: 156 - protein, 209 aa, Artificial Sequence
  Description of Artificial Sequence Synthetic polypeptide
LEPGEKPYKCPECGKSFSQSGDLRRHQRTHTGEKPYKCPECGKSFSQSGDLRRHQRTHTGEKPYKCPECGKSFSERSHLREHQRTHTGEKPYKCPECGKSFSTTGNLTVHQRTHTGEKPYKCPECGKSFSHRTTLTNHQRTHTGEKPYKCPECGKSFSDSGNLRVHQRTHTGEKPYKCPECGKSFSQSSSLVRHQRTHTGEKPTGKKTS

SEQ ID NO: 157 - DNA, 627 bp, Artificial Sequence
  Description of Artificial Sequence Synthetic polynucleotide
cttgagcccggagaaaagccatacaaatgtcctgaatgcggaaagtcattttctacgagcggcgaactcgtgcggcaccaaaggactcataccggcgaaaagccttacaaatgcccggagtgcggaaaaagcttctccgagcgctcgcacttgcgggaacaccagcgaacccacacaggggagaaaccgtataagtgcccagagtgcggcaaatcgttctcccggaacgacaccctgaccgaacaccaacgcactcatactggcgaaaaaccttacaagtgccctgagtgtggaaagagcttctcccgcgccgataacctgaccgagcaccagcggacccataccggggaaaagccgtacaagtgtccggaatgcggcaaaagcttcagcacctcgggttccctggtccggcatcagagaactcacaccggagagaaaccctataagtgtcctgagtgcgggaagtccttttcatcgcccgcggacctgactagacaccagaggacccacaccggggagaagccctacaagtgccccgaatgtggaaagtccttctccgactccggcaacctccgggtgcaccagcgcacccacactggagagaagccgaccggaaagaaaacttcc

SEQ ID NO: 158 - DNA, 627 bp, Artificial Sequence
  Description of Artificial Sequence Synthetic polynucleotide
ctggaacccggagaaaaaccttataagtgccctgaatgcggaaagtcattctcgaggtcggacaagctcgtgcggcaccagaggacacacaccggggaaaagccatacaagtgtcccgaatgtggaaagtccttcagccaacgcgccaacctgagagctcatcagcggactcacactggcgaaaaaccgtacaaatgccccgaatgcggcaaaagcttctcccgcgccgacaacttgaccgagcaccagcggacccataccggcgaaaagccgtacaagtgcccggagtgtgggaagtcgttcagccagtcctcttccctcgtgcgccaccaacgcacccatactggggagaagccctataagtgtcctgagtgtggcaaatcattcagcgataagaaggatcttacccggcaccaacggactcataccggagagaagccttacaagtgccccgagtgcggaaagagcttctcgtccccggcggacctgactagacaccagcgcacccacaccggagaaaagccctacaagtgcccagagtgcgggaagtccttttcccaatccggtcacctgactgagcaccagagaacccacacgggagagaaaccgaccggaaagaaaacctcc

SEQ ID NO: 159 - DNA, 627 bp, Artificial Sequence
  Description of Artificial Sequence Synthetic polynucleotide
ttggaacccggagaaaagccatacaaatgccccgaatgcggaaagtcgttcagccagtccggcgacctcagacggcaccaacggactcacaccggcgaaaaaccgtacaagtgcccagagtgcggcaaaagctttagccagtcgggcgatctgcggagacatcagcgcactcacactggtgaaaagccctacaagtgtcctgagtgcgggaagtccttcagcgagcgctcccatcttcgcgagcaccagagaacccacactggagaaaaaccttataagtgccctgagtgtggcaaatccttctcaaccaccggcaacctgactgtgcaccagcggacccacacaggggagaagccttacaagtgcccggagtgtgggaagtcattctcccatcggacgaccctgaccaaccaccagaggacccatactggcgaaaagccgtataagtgtccggagtgcggaaagagcttctccgactccggaaacctcagggtgcaccaacgcacccacaccggagagaagccgtacaaatgtcccgaatgtggaaagtccttctcccaatcctcttcgctggtccggcaccagcgaactcataccggggaaaagcccaccggaaagaaaacctcg

SEQ ID NO: 160 - protein, 659 aa, Artificial Sequence
  Description of Artificial Sequence Synthetic polypeptide
MAPKKKRKVGIHGVPAAGSSGSLEPGEKPYKCPECGKSFSTSGELVRHQRTHTGEKPYKCPECGKSFSERSHLREHQRTHTGEKPYKCPECGKSFSRNDTLTEHQRTHTGEKPYKCPECGKSFSRADNLTEHQRTHTGEKPYKCPECGKSFSTSGSLVRHQRTHTGEKPYKCPECGKSFSSPADLTRHQRTHTGEKPYKCPECGKSFSDSGNLRVHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGSYPYDVPDYA

SEQ ID NO: 161 - protein, 659 aa, Artificial Sequence
  Description of Artificial Sequence Synthetic polypeptide
MAPKKKRKVGIHGVPAAGSSGSLEPGEKPYKCPECGKSFSRSDKLVRHQRTHTGEKPYKCPECGKSFSQRANLRAHQRTHTGEKPYKCPECGKSFSRADNLTEHQRTHTGEKPYKCPECGKSFSQSSSLVRHQRTHTGEKPYKCPECGKSFSDKKDLTRHQRTHTGEKPYKCPECGKSFSSPADLTRHQRTHTGEKPYKCPECGKSFSQSGHLTEHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGSYPYDVPDYA

SEQ ID NO: 162 - protein, 659 aa, Artificial Sequence
  Description of Artificial Sequence Synthetic polypeptide
MAPKKKRKVGIHGVPAAGSSGSLEPGEKPYKCPECGKSFSQSGDLRRHQRTHTGEKPYKCPECGKSFSQSGDLRRHQRTHTGEKPYKCPECGKSFSERSHLREHQRTHTGEKPYKCPECGKSFSTTGNLTVHQRTHTGEKPYKCPECGKSFSHRTTLTNHQRTHTGEKPYKCPECGKSFSDSGNLRVHQRTHTGEKPYKCPECGKSFSQSSSLVRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGSYPYDVPDYA

SEQ ID NO: 163 - protein, 650 aa, Artificial Sequence
  Description of Artificial Sequence Synthetic polypeptide
MAPKKKRKVGIHGVPAAGSSGSLEPGEKPYKCPECGKSFSTSGELVRHQRTHTGEKPYKCPECGKSFSERSHLREHQRTHTGEKPYKCPECGKSFSRNDTLTEHQRTHTGEKPYKCPECGKSFSRADNLTEHQRTHTGEKPYKCPECGKSFSTSGSLVRHQRTHTGEKPYKCPECGKSFSSPADLTRHQRTHTGEKPYKCPECGKSFSDSGNLRVHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGS

SEQ ID NO: 164 - protein, 650 aa, Artificial Sequence
  Description of Artificial Sequence Synthetic polypeptide
MAPKKKRKVGIHGVPAAGSSGSLEPGEKPYKCPECGKSFSRSDKLVRHQRTHTGEKPYKCPECGKSFSQRANLRAHQRTHTGEKPYKCPECGKSFSRADNLTEHQRTHTGEKPYKCPECGKSFSQSSSLVRHQRTHTGEKPYKCPECGKSFSDKKDLTRHQRTHTGEKPYKCPECGKSFSSPADLTRHQRTHTGEKPYKCPECGKSFSQSGHLTEHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGS

SEQ ID NO: 165 - protein, 650 aa, Artificial Sequence
  Description of Artificial Sequence Synthetic polypeptide
MAPKKKRKVGIHGVPAAGSSGSLEPGEKPYKCPECGKSFSQSGDLRRHQRTHTGEKPYKCPECGKSFSQSGDLRRHQRTHTGEKPYKCPECGKSFSERSHLREHQRTHTGEKPYKCPECGKSFSTTGNLTVHQRTHTGEKPYKCPECGKSFSHRTTLTNHQRTHTGEKPYKCPECGKSFSDSGNLRVHQRTHTGEKPYKCPECGKSFSQSSSLVRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGS

SEQ ID NO: 166 - DNA, 2255 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatcccttgagcccggagaaaagccatacaaatgtcctgaatgcggaaagtcattttctacgagcggcgaactcgtgcggcaccaaaggactcataccggcgaaaagccttacaaatgcccggagtgcggaaaaagcttctccgagcgctcgcacttgcgggaacaccagcgaacccacacaggggagaaaccgtataagtgcccagagtgcggcaaatcgttctcccggaacgacaccctgaccgaacaccaacgcactcatactggcgaaaaaccttacaagtgccctgagtgtggaaagagcttctcccgcgccgataacctgaccgagcaccagcggacccataccggggaaaagccgtacaagtgtccggaatgcggcaaaagcttcagcacctcgggttccctggtccggcatcagagaactcacaccggagagaaaccctataagtgtcctgagtgcgggaagtccttttcatcgcccgcggacctgactagacaccagaggacccacaccggggagaagccctacaagtgccccgaatgtggaaagtccttctccgactccggcaacctccgggtgcaccagcgcacccacactggagagaagccgaccggaaagaaaacttccgcctccggttcgggaggaggctcaggaggagcgagagattccaaggtcgagaacaagaccaagaagctgcgggtgttcgaggcctttgctggcatcggagcccagaggaaggccctcgagaaggtccgcaaggatgagtacgagatcgtgggactcgcggagtggtacgtgcccgccattgtgatgtaccaggccatccataacaacttccacactaagctggagtacaagtccgtgtcccgggaggaaatgattgactacctggagaataagaccctgtcatggaactctaagaaccccgtgtcgaacggttactggaagagaaagaaggatgacgaactgaagattatctacaacgcgatcaagctgagcgagaaggagggcaacatcttcgacatccgggacctctacaagcgcaccttgaagaacatcgatctgctgacctactccttcccgtgccaagacctgagccagcagggcatccagaaggggatgaaacggggctccggtactcgcagcggcttgctgtgggaaattgagcgggccctggatagcaccgagaagaacgacttgccgaagtatcttctcatggaaaacgtcggggctctccttcacaagaagaacgaggaagaactgaaccagtggaagcaaaagctggaatccctcggataccagaactccattgaggtcctgaacgccgccgacttcggatcgtcgcaagccagacggagggtgttcatgattagcactctgaacgaattcgtggaactgccgaagggcgacaagaagcctaagtccatcaagaaggtgctgaacaagatcgtgtccgagaaggacattctcaacaatctgctgaagtacaacctgacagagttcaagaaaaccaagtccaacatcaacaaggcctccttgattggttactcaaagttcaacagcgagggatacgtgtacgaccccgaattcactggacccactctgaccgcctccggagcaaactctaggattaagatcaaggacggctccaacatccggaagatgaactccgacgaaacctttctgtacatcggcttcgactcgcaagacggaaagcgcgtgaacgagatcgaatttcttaccgaaaaccagaagatcttcgtgtgcggcaattcaatctccgtggaagtcctggaagcgattatcgacaagatcggaggcagtggtggaaagcgcccagcagccactaagaaggccggacaggccaagaagaagaagggatcctacccttacgatgtgccggattacgcttgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 167 - DNA, 2255 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggaacccggagaaaaaccttataagtgccctgaatgcggaaagtcattctcgaggtcggacaagctcgtgcggcaccagaggacacacaccggggaaaagccatacaagtgtcccgaatgtggaaagtccttcagccaacgcgccaacctgagagctcatcagcggactcacactggcgaaaaaccgtacaaatgccccgaatgcggcaaaagcttctcccgcgccgacaacttgaccgagcaccagcggacccataccggcgaaaagccgtacaagtgcccggagtgtgggaagtcgttcagccagtcctcttccctcgtgcgccaccaacgcacccatactggggagaagccctataagtgtcctgagtgtggcaaatcattcagcgataagaaggatcttacccggcaccaacggactcataccggagagaagccttacaagtgccccgagtgcggaaagagcttctcgtccccggcggacctgactagacaccagcgcacccacaccggagaaaagccctacaagtgcccagagtgcgggaagtccttttcccaatccggtcacctgactgagcaccagagaacccacacgggagagaaaccgaccggaaagaaaacctccgcctccggttcgggaggaggctcaggaggagcgagagattccaaggtcgagaacaagaccaagaagctgcgggtgttcgaggcctttgctggcatcggagcccagaggaaggccctcgagaaggtccgcaaggatgagtacgagatcgtgggactcgcggagtggtacgtgcccgccattgtgatgtaccaggccatccataacaacttccacactaagctggagtacaagtccgtgtcccgggaggaaatgattgactacctggagaataagaccctgtcatggaactctaagaaccccgtgtcgaacggttactggaagagaaagaaggatgacgaactgaagattatctacaacgcgatcaagctgagcgagaaggagggcaacatcttcgacatccgggacctctacaagcgcaccttgaagaacatcgatctgctgacctactccttcccgtgccaagacctgagccagcagggcatccagaaggggatgaaacggggctccggtactcgcagcggcttgctgtgggaaattgagcgggccctggatagcaccgagaagaacgacttgccgaagtatcttctcatggaaaacgtcggggctctccttcacaagaagaacgaggaagaactgaaccagtggaagcaaaagctggaatccctcggataccagaactccattgaggtcctgaacgccgccgacttcggatcgtcgcaagccagacggagggtgttcatgattagcactctgaacgaattcgtggaactgccgaagggcgacaagaagcctaagtccatcaagaaggtgctgaacaagatcgtgtccgagaaggacattctcaacaatctgctgaagtacaacctgacagagttcaagaaaaccaagtccaacatcaacaaggcctccttgattggttactcaaagttcaacagcgagggatacgtgtacgaccccgaattcactggacccactctgaccgcctccggagcaaactctaggattaagatcaaggacggctccaacatccggaagatgaactccgacgaaacctttctgtacatcggcttcgactcgcaagacggaaagcgcgtgaacgagatcgaatttcttaccgaaaaccagaagatcttcgtgtgcggcaattcaatctccgtggaagtcctggaagcgattatcgacaagatcggaggcagtggtggaaagcgcccagcagccactaagaaggccggacaggccaagaagaagaagggatcctacccttacgatgtgccggattacgcttgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 168 - DNA, 2255 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccttggaacccggagaaaagccatacaaatgccccgaatgcggaaagtcgttcagccagtccggcgacctcagacggcaccaacggactcacaccggcgaaaaaccgtacaagtgcccagagtgcggcaaaagctttagccagtcgggcgatctgcggagacatcagcgcactcacactggtgaaaagccctacaagtgtcctgagtgcgggaagtccttcagcgagcgctcccatcttcgcgagcaccagagaacccacactggagaaaaaccttataagtgccctgagtgtggcaaatccttctcaaccaccggcaacctgactgtgcaccagcggacccacacaggggagaagccttacaagtgcccggagtgtgggaagtcattctcccatcggacgaccctgaccaaccaccagaggacccatactggcgaaaagccgtataagtgtccggagtgcggaaagagcttctccgactccggaaacctcagggtgcaccaacgcacccacaccggagagaagccgtacaaatgtcccgaatgtggaaagtccttctcccaatcctcttcgctggtccggcaccagcgaactcataccggggaaaagcccaccggaaagaaaacctcggcctccggttcgggaggaggctcaggaggagcgagagattccaaggtcgagaacaagaccaagaagctgcgggtgttcgaggcctttgctggcatcggagcccagaggaaggccctcgagaaggtccgcaaggatgagtacgagatcgtgggactcgcggagtggtacgtgcccgccattgtgatgtaccaggccatccataacaacttccacactaagctggagtacaagtccgtgtcccgggaggaaatgattgactacctggagaataagaccctgtcatggaactctaagaaccccgtgtcgaacggttactggaagagaaagaaggatgacgaactgaagattatctacaacgcgatcaagctgagcgagaaggagggcaacatcttcgacatccgggacctctacaagcgcaccttgaagaacatcgatctgctgacctactccttcccgtgccaagacctgagccagcagggcatccagaaggggatgaaacggggctccggtactcgcagcggcttgctgtgggaaattgagcgggccctggatagcaccgagaagaacgacttgccgaagtatcttctcatggaaaacgtcggggctctccttcacaagaagaacgaggaagaactgaaccagtggaagcaaaagctggaatccctcggataccagaactccattgaggtcctgaacgccgccgacttcggatcgtcgcaagccagacggagggtgttcatgattagcactctgaacgaattcgtggaactgccgaagggcgacaagaagcctaagtccatcaagaaggtgctgaacaagatcgtgtccgagaaggacattctcaacaatctgctgaagtacaacctgacagagttcaagaaaaccaagtccaacatcaacaaggcctccttgattggttactcaaagttcaacagcgagggatacgtgtacgaccccgaattcactggacccactctgaccgcctccggagcaaactctaggattaagatcaaggacggctccaacatccggaagatgaactccgacgaaacctttctgtacatcggcttcgactcgcaagacggaaagcgcgtgaacgagatcgaatttcttaccgaaaaccagaagatcttcgtgtgcggcaattcaatctccgtggaagtcctggaagcgattatcgacaagatcggaggcagtggtggaaagcgcccagcagccactaagaaggccggacaggccaagaagaagaagggatcctacccttacgatgtgccggattacgcttgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 169 - protein, 204 aa, Artificial Sequence
  Description of Artificial Sequence Synthetic polypeptide
LEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTS

SEQ ID NO: 170 - protein, 204 aa, Artificial Sequence
  Description of Artificial Sequence Synthetic polypeptide
LEPGEKPYKCPECGKSFSQKSSLIAHQRTHTGEKPYKCPECGKSFSHKNALQNHQRTHTGEKPYKCPECGKSFSQSSNLVRHQRTHTGEKPYKCPECGKSFSRNDALTEHQRTHTGEKPYKCPECGKSFSDKKDLTRHQRTHTGEKPYKCPECGKSFSQAGHLASHQRTHTGEKPYKCPECGKSFSDKKDLTRHQRTHTGKKTS

SEQ ID NO: 171 - protein, 204 aa, Artificial Sequence
  Description of Artificial Sequence Synthetic polypeptide
LEPGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGEKPYKCPECGKSFSREDNLHTHQRTHTGKKTS

SEQ ID NO: 172 - protein, 204 aa, Artificial Sequence
  Description of Artificial Sequence Synthetic polypeptide
LEPGEKPYKCPECGKSFSRSDHLTTHQRTHTGEKPYKCPECGKSFSQKSSLIAHQRTHTGEKPYKCPECGKSFSRRDELNVHQRTHTGEKPYKCPECGKSFSQSGDLRRHQRTHTGEKPYKCPECGKSFSQAGHLASHQRTHTGEKPYKCPECGKSFSTSGNLTEHQRTHTGEKPYKCPECGKSFSQAGHLASHQRTHTGKKTS

SEQ ID NO: 173 - DNA, 612 bp, Artificial Sequence
  Description of Artificial Sequence Synthetic polynucleotide
ctggagcctggagagaaaccctacaaatgcccggaatgcgggaagtccttttccgaacgctcgcacctgagggaacaccagagaactcacaccggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccatcagcgcacccataccggcaaaaagacctct

SEQ ID NO: 174 - DNA, 612 bp, Artificial Sequence
  Description of Artificial Sequence Synthetic polynucleotide
cttgaacccggggagaagccctacaagtgcccggaatgcggaaagagcttcagccagaagtcctcgctgatcgcgcaccagaggactcacaccggcgaaaagccatacaagtgtcctgagtgtggcaaatccttctcgcacaagaacgcactgcagaaccaccagagaacccacaccggggaaaagccgtataagtgccccgaatgtggaaagtcgttcagccagtcatccaacctcgtgcgccatcaaaggactcataccggagagaaaccttacaaatgccctgaatgcggcaaatctttctcccggaatgatgccctgaccgagcaccagcgcactcacacgggagagaagccgtacaaatgtccggagtgcggaaagtccttctccgacaagaaggacttgaccagacaccagcggacccatactggcgaaaaaccctataagtgtccagagtgcgggaagtcctttagccaagccggtcacctcgcttcccaccaacggacccacacaggagaaaagccttataaatgccccgagtgcggcaaaagcttctccgataagaaggacctgactcggcatcagcgcacccataccggaaagaaaacctca

SEQ ID NO: 175 - DNA, 612 bp, Artificial Sequence
  Description of Artificial Sequence Synthetic polynucleotide
ctggagcctggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccaccagagaacccatactggcgagaagccatacaaatgcccggaatgtggaaagagtttctcgcgcgaggacaacctccacacccatcagcgcacccataccggcaaaaagacctct

SEQ ID NO: 176 - DNA, 612 bp, Artificial Sequence
  Description of Artificial Sequence Synthetic polynucleotide
ctggaacccggagagaaaccctacaaatgcccagagtgcggcaaatcgttctcacggtccgatcacctcaccacccaccagaggacccataccggggagaagccttacaagtgtcctgagtgtggaaagtccttcagccaaaagtcctcgctgatcgcacaccagcgcacgcacactggggaaaagccatataaatgcccggagtgtggcaaatccttctcccgccgcgacgaactgaacgtgcaccagagaacccacactggagagaagccgtataagtgtccggagtgcggaaagagcttctcgcaatccggggaccttcggagacatcagaggacacacactggcgaaaagccctataagtgccctgagtgcgggaagtcctttagccaagccggtcacctggcctcccaccaacggactcacaccggcgaaaaaccgtacaagtgccccgaatgcggaaagtcgttctctacctccggaaacttgaccgaacaccagcggacccacaccggagaaaagccgtacaaatgtcctgaatgcggcaaaagcttcagccaggccggtcatctcgcgagccatcagcggactcatactggcaaaaagacctca

SEQ ID NO: 177 - protein, 367 aa, Artificial Sequence
  Description of Artificial Sequence Synthetic polypeptide
MAPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 178 - protein, 367 aa, Artificial Sequence
  Description of Artificial Sequence Synthetic polypeptide
MAPKKKRKVGIHGVPAAGSSGSLEPGEKPYKCPECGKSFSQKSSLIAHQRTHTGEKPYKCPECGKSFSHKNALQNHQRTHTGEKPYKCPECGKSFSQSSNLVRHQRTHTGEKPYKCPECGKSFSRNDALTEHQRTHTGEKPYKCPECGKSFSDKKDLTRHQRTHTGEKPYKCPECGKSFSQAGHLASHQRTHTGEKPYKCPECGKSFSDKKDLTRHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 179 - protein, 367 aa, Artificial Sequence
  Description of Artificial Sequence Synthetic polypeptide
MAPKKKRKVGIHGVPAAGSSGSLEPGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGEKPYKCPECGKSFSREDNLHTHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 180 - protein, 367 aa, Artificial Sequence
  Description of Artificial Sequence Synthetic polypeptide
MAPKKKRKVGIHGVPAAGSSGSLEPGEKPYKCPECGKSFSRSDHLTTHQRTHTGEKPYKCPECGKSFSQKSSLIAHQRTHTGEKPYKCPECGKSFSRRDELNVHQRTHTGEKPYKCPECGKSFSQSGDLRRHQRTHTGEKPYKCPECGKSFSQAGHLASHQRTHTGEKPYKCPECGKSFSTSGNLTEHQRTHTGEKPYKCPECGKSFSQAGHLASHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 181 - protein, 1082 aa, Artificial Sequence
  Description of Artificial Sequence Synthetic polypeptide
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYAGSATNFSLLKQAGDVEENPGPTSAGKLGSGEGRGSLLTCGDVEENPGPLEGSSGSGSPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 182 - protein, 1082 aa, Artificial Sequence
  Description of Artificial Sequence Synthetic polypeptide
MAPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYAGSATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYA

SEQ ID NO: 183 - protein, 358 aa, Artificial Sequence
  Description of Artificial Sequence Synthetic polypeptide
MAPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 184 - protein, 358 aa, Artificial Sequence
  Description of Artificial Sequence Synthetic polypeptide
MAPKKKRKVGIHGVPAAGSSGSLEPGEKPYKCPECGKSFSQKSSLIAHQRTHTGEKPYKCPECGKSFSHKNALQNHQRTHTGEKPYKCPECGKSFSQSSNLVRHQRTHTGEKPYKCPECGKSFSRNDALTEHQRTHTGEKPYKCPECGKSFSDKKDLTRHQRTHTGEKPYKCPECGKSFSQAGHLASHQRTHTGEKPYKCPECGKSFSDKKDLTRHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 185 - protein, 358 aa, Artificial Sequence
  Description of Artificial Sequence Synthetic polypeptide
MAPKKKRKVGIHGVPAAGSSGSLEPGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGEKPYKCPECGKSFSREDNLHTHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 186 - protein, 358 aa, Artificial Sequence
  Description of Artificial Sequence Synthetic polypeptide
MAPKKKRKVGIHGVPAAGSSGSLEPGEKPYKCPECGKSFSRSDHLTTHQRTHTGEKPYKCPECGKSFSQKSSLIAHQRTHTGEKPYKCPECGKSFSRRDELNVHQRTHTGEKPYKCPECGKSFSQSGDLRRHQRTHTGEKPYKCPECGKSFSQAGHLASHQRTHTGEKPYKCPECGKSFSTSGNLTEHQRTHTGEKPYKCPECGKSFSQAGHLASHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 187 - protein, 1073 aa, Artificial Sequence
  Description of Artificial Sequence Synthetic polypeptide
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYAGSATNFSLLKQAGDVEENPGPTSAGKLGSGEGRGSLLTCGDVEENPGPLEGSSGSGSPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 188 - protein, 1073 aa, Artificial Sequence
  Description of Artificial Sequence Synthetic polypeptide
MAPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYAGSATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGS

SEQ ID NO: 189 - DNA, 1376 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcctggagagaaaccctacaaatgcccggaatgcgggaagtccttttccgaacgctcgcacctgagggaacaccagagaactcacaccggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccatcagcgcacccataccggcaaaaagacctctgctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 190 - DNA, 21 bp, Homo sapiens
aacacagttcagccgagcgct

SEQ ID NO: 191 - DNA, 21 bp, Homo sapiens
cgaacaaccgtacagaaaggg

SEQ ID NO: 192 - DNA, 21 bp, Homo sapiens
gtaaacagtaatagcgcagca

SEQ ID NO: 193 - DNA, 1376 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatcccttgaacccggggagaagccctacaagtgcccggaatgcggaaagagcttcagccagaagtcctcgctgatcgcgcaccagaggactcacaccggcgaaaagccatacaagtgtcctgagtgtggcaaatccttctcgcacaagaacgcactgcagaaccaccagagaacccacaccggggaaaagccgtataagtgccccgaatgtggaaagtcgttcagccagtcatccaacctcgtgcgccatcaaaggactcataccggagagaaaccttacaaatgccctgaatgcggcaaatctttctcccggaatgatgccctgaccgagcaccagcgcactcacacgggagagaagccgtacaaatgtccggagtgcggaaagtccttctccgacaagaaggacttgaccagacaccagcggacccatactggcgaaaaaccctataagtgtccagagtgcgggaagtcctttagccaagccggtcacctcgcttcccaccaacggacccacacaggagaaaagccttataaatgccccgagtgcggcaaaagcttctccgataagaaggacctgactcggcatcagcgcacccataccggaaagaaaacctcagctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 194 - DNA, 1376 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggagcctggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccaccagagaacccatactggcgagaagccatacaaatgcccggaatgtggaaagagtttctcgcgcgaggacaacctccacacccatcagcgcacccataccggcaaaaagacctctgctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 195 - DNA, 1376 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccggcagcagcggatccctggaacccggagagaaaccctacaaatgcccagagtgcggcaaatcgttctcacggtccgatcacctcaccacccaccagaggacccataccggggagaagccttacaagtgtcctgagtgtggaaagtccttcagccaaaagtcctcgctgatcgcacaccagcgcacgcacactggggaaaagccatataaatgcccggagtgtggcaaatccttctcccgccgcgacgaactgaacgtgcaccagagaacccacactggagagaagccgtataagtgtccggagtgcggaaagagcttctcgcaatccggggaccttcggagacatcagaggacacacactggcgaaaagccctataagtgccctgagtgcgggaagtcctttagccaagccggtcacctggcctcccaccaacggactcacaccggcgaaaaaccgtacaagtgccccgaatgcggaaagtcgttctctacctccggaaacttgaccgaacaccagcggacccacaccggagaaaagccgtacaaatgtcctgaatgcggcaaaagcttcagccaggccggtcatctcgcgagccatcagcggactcatactggcaaaaagacctcagctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 196 - DNA, 3521 bp, Artificial Sequence
  Description of Artificial Sequence Synthetic polynucleotide
gggaaataagagagaaaagaagagtaagaagaaatataagagccaccatggccccaaagaagaagagaaaggtcggaattcatggcgtgcccgcagccggcagcagcggttccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcggcggcaagcggcccgccgccaccaagaaggccggccaggccaagaagaagaagggcagctacccctacgacgtgcccgactacgccgggtccgccaccaacttctcgctgctgaagcaggccggagacgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcctggagagaaaccctacaaatgcccggaatgcgggaagtccttttccgaacgctcgcacctgagggaacaccagagaactcacaccggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccatcagcgcacccataccggcaaaaagacctctgctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 197 - DNA, 3521 bp, Artificial Sequence
  Description of Artificial Sequence Synthetic polynucleotide
gggaaataagagagaaaagaagagtaagaagaaatataagagccaccatggccccaaagaagaagagaaaggtcggaattcatggcgtgcccgcagccggcagcagcggttccctggagcctggagagaaaccctacaaatgcccggaatgcgggaagtccttttccgaacgctcgcacctgagggaacaccagagaactcacaccggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccatcagcgcacccataccggcaaaaagacctctgctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgccgggtccgccaccaacttctcgctgctgaagcaggccggagacgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 198 - DNA, 1385 bp, Artificial Sequence
  Description of Artificial Sequence Synthetic polynucleotide
gaaataagagagaaaagaagagtaagaagaaatataagacctaatagtaatagtaatagcccaagaagaagcgcaaggtcgggatccacggagtcccggcagcaggatcctcaggctcactggaaccgggggaaaaaccctacaagtgcccggagtgcggcaagagcttctcgacctccgggaacctgaccgagcaccagcgcacccacaccggagagaaaccgtacaagtgccccgagtgcgggaaatcgttctcagactcgggaaacctcagggtgcaccagcggacccacacgggggaaaagccgtacaagtgcccggagtgcgggaagtcattctcccacaagaacgcgctgcagaaccaccaaagaacccacaccggcgaaaaaccgtacaagtgccccgagtgcggaaagtccttctcccgcaacgacaccctcaccgaacaccaacgcacccacaccggagaaaagccctacaagtgcccggagtgcggaaagagcttcagccagagggcccacctggaaagacaccagagaacccacaccggcgaaaagccgtacaagtgcccggagtgcgggaagtccttcagccggtcagacaagctggtccgccaccaaaggacccacacaggagaaaagccctacaagtgcccggagtgcggaaaatcgttcagcgaccccggacacctggtccggcaccagaggacccacaccggggagaagccgaccggcaaaaagacctcagcaagcgggagcggaggaggaagcggaggggacgccaagagcctgaccgcgtggagcaggaccctcgtgaccttcaaagacgtgttcgtggacttcacccgcgaagagtggaagctgctggacaccgcgcagcaaatcctgtaccggaacgtgatgctggaaaactacaagaacctcgtcagcctcggctaccaactgaccaagcccgacgtgatcctgcggctggaaaagggcgaagaaccctggctcgtggagcgggagatccaccaggaaacccacccggacagcgaaaccgcgttcgaaatcaagagcagcgtcagcggaggaaaaagaccagccgccaccaagaaggccggccaagccaagaagaaaaagggcagctacccctacgacgtgccggactacgcatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 199 - DNA, 21 bp, Homo sapiens
acggggaatgctgccgagagc

SEQ ID NO: 200 - DNA, 21 bp, Homo sapiens
acctgaaccctggaaattata

SEQ ID NO: 201 - DNA, 21 bp, Homo sapiens
tagacggggaatgctgccgag

SEQ ID NO: 202 - DNA, 21 bp, Homo sapiens
tgacattgagcaatgatatgg

SEQ ID NO: 203 - DNA, 21 bp, Homo sapiens
agcaaaagaaaatggtaggcg

SEQ ID NO: 204 - DNA, 21 bp, Homo sapiens
acggggaatgctgccgagagc

SEQ ID NO: 205 - DNA, 21 bp, Homo sapiens
acggggaatgctgccgagagc

SEQ ID NO: 206 - DNA, 21 bp, Homo sapiens
agcaaaagaaaatggtaggcg

SEQ ID NO: 207 - protein, 5 aa, Artificial Sequence
  Description of Artificial Sequence Synthetic peptide
GGGGS

SEQ ID NO: 208 - DNA, 830 bp, Artificial Sequence
  Description of Artificial Sequence Synthetic polynucleotide
gatgttcattagcagtggtgataggttaattttcaccatctcttatgcggttgaatagtcacctctgaaccactttttcctccagtaactcctctttcttcggaccttctgcagccaacctgaaagaataacaaggaggtggctggaaacttgttttaaggaaccgcctgtccttcccccgctggaaaccttgcacctcggacgctcctgctcctgcccccacctgacccccgccctcgttgacatccaggcgcgatgatctctgctgccagtagagggcacacttactttactttcgcaaacctgaacgcgggtgctgcccagagagggggcggagggaaagacgctttgcagcaaaatccagcatagcgattggttgctccccgcgtttgcggcaaaggcctggaggcaggagtaatttgcaatccttaaagctgaattgtgcagtgcatcggatttggaagctactatattcacttaacacttgaacgctgagctgcaaactcaacgggtaataacccatcttgaacagcgtacatgctatacacgcacccctttcccccgaattgttttctcttttggaggtggtggagggagagaaaagtttacttaaaatgcctttgggtgagggaccaaggatgagaagaatgttttttgtttttcatgccgtggaataacacaaaataaaaaatcccgagggaatatacattatatattaaatatagatcatttcagggagcaaacaaatcatgtgtggggctgggcaactagctaagtcgaagcgtaaataaaatgtgaatacacgtttgcgggttacatacagtgcactt

SEQ ID NO: 209 - DNA, 830 bp, Artificial Sequence
  Description of Artificial Sequence Synthetic polynucleotide
taggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgg

SEQ ID NO: 210 - DNA, 7 bp, Artificial Sequence
  Description of Artificial Sequence Synthetic oligonucleotide
tatawaw

(19) United States
(12) Patent Application Publication
(10) Pub. No.: US 2024/0132559 A1
(43) Pub. Date: Apr. 25, 2024

(54) COMPOSITIONS AND METHODS FOR MODULATING MYC EXPRESSION

(71) Applicant: FLAGSHIP PIONEERING INNOVATIONS V, INC., Cambridge, MA (US)

(72) Inventors: Abigail Elizabeth WITT, Charlestown, MA (US); Jeremiah Dale FARELLI, Marblehead, MA (US); Adam Walter SCHEIDEGGER, Somerville, MA (US); William Thomas SENAPEDIS, Jr., Millis, MA (US); Jodi Michelle KENNEDY, Dedham, MA (US); Houda BELAGHZAL, Cambridge, MA (US); Defne YARAR, Brookline, MA (US); Eugine LEE, Cambridge, MA (US)

(21) Appl. No.: 18/257,483

(22) PCT Filed: Dec. 15, 2021
(86) PCT No.: PCT/US21/10059

§ 371 Date: Jun. 14, 2023

Related U.S. Application Data
(60) Provisional application No. 63/125,833, filed on Dec. 15, 2020.
(60) Provisional application No. 63/137,097, filed on Jan. 13, 2021.
(60) Provisional application No. 63/212,991, filed on Jun. 21, 2021.
(60) Provisional application No. 63/281,022, filed on Nov. 18, 2021.

(51) Int. Cl. C07K 14/47; A61K 45/06; A61P 31/14; A61P 35/00; C12N 9/10

(52) U.S. Cl. CPC C07K 14/4703; A61K 45/06; A61P 31/14; A61P 35/00; C12N 9/1007; A61K 38/00

(57) ABSTRACT

The present disclosure relates to compositions and methods for reducing expression of MYC gene in a cell. In some embodiments, an expression repressor comprises a targeting moiety that binds a MYC promoter, anchor sequence, or super-enhancer. In some embodiments, the expression repressor comprises an effector moiety that represses transcription or methylates DNA. Systems comprising two expression repressors are also disclosed. The compositions can be used, for example, to treat cancers such as HCC or NSCLC.

### CROSS-REFERENCE TO RELATED APPLICATIONS

This application claims priority to U.S. Provisional Application 63/125,833 filed on Dec. 15, 2020, U.S. Provisional Application 63/137,097 filed on Jan. 13, 2021, U.S. Provisional Application 63/212,991 filed on Jun. 21, 2021, and U.S. Provisional Application 63/281,022 filed on Nov. 18, 2021, the entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on Dec. 6, 2021, is named O2057-7029WO_SL.txt and is 624,274 bytes in size.

### BACKGROUND

Mis-regulation of gene expression is the underlying cause of many diseases (e.g., in mammals, e.g., humans) e.g., neoplasia, neurological disorders, metabolic disorders and obesity. The mis-regulation of the transcription factor MYC plays a central role in a variety of human tumors and chronic liver diseases. MYC protein is considered “undruggable” due to various factors, e.g., lack of a defined ligand binding site, physiological function essential to the maintenance of normal tissues. Techniques geared towards modulating the MYC gene expression provides a viable alternative approach in treating these diseases. There is a need for novel tools, systems, and methods to stably alter, e.g., decrease, expression of disease associated genes such as MYC.

### SUMMARY

The disclosure provides, among other things, expression repressors and expression repressor systems that may be used to modulate, e.g., decrease, expression of a target gene, e.g., MYC.

In some aspects, the disclosure provides an expression repressor comprises a targeting moiety that binds to a target gene promoter, e.g., MYC promoter, and optionally, an effector moiety, wherein the expression repressor is capable of decreasing expression of the target gene, e.g., MYC.

In some aspects, the disclosure provides an expression repressor comprising: a targeting moiety that binds a target gene locus, e.g., MYC, and an effector moiety comprising MQ1 or a fragment or variant thereof, wherein the expression repressor is capable of decreasing expression of target gene, e.g., MYC.

In some aspects, the disclosure provides an expression repressor comprising: a targeting moiety that binds to a regulatory element located in a super enhancer region of MYC, and optionally an effector moiety wherein the expression repressor is capable of decreasing expression of MYC.

In some aspects, the disclosure provides an expression repressor comprising: a targeting moiety that binds to a regulatory element located in a super enhancer region of a target gene, e.g., MYC, and an effector moiety (e.g., KRAB, or MQ1, or a fragment or variant thereof) wherein the expression repressor is capable of decreasing expression of the target gene, e.g., MYC.

In some aspects, the disclosure provides an expression repressor comprising: a targeting moiety that binds a regulatory element located in a super enhancer region of a target gene, e.g., MYC, wherein the targeting moiety comprises a zinc finger domain, wherein the expression repressor is capable of decreasing expression of target gene, e.g., MYC.

In some aspects, the disclosure provides an expression repressor comprising: a targeting moiety that binds a regulatory element located in a super enhancer region of MYC, wherein the targeting moiety comprises a zinc finger domain or a TAL effector domain, and an effector moiety, wherein the effector moiety comprises a transcription repressor (e.g., KRAB or a fragment or variant thereof) or a DNA methyltransferase (e.g., MQ1 or a fragment or variant thereof); wherein the expression repressor is capable of decreasing expression of MYC.

In some aspects, the disclosure provides an expression repressor comprising: a targeting moiety that binds a target gene locus, e.g., MYC, wherein the targeting moiety comprises a zinc finger domain, wherein the expression repressor is capable of decreasing expression of target gene, e.g., MYC.

In some aspects, the disclosure provides expression repressor comprising: a targeting moiety that binds a genomic locus comprising at least 14, 15, 16, 17, 18, 19, or 20 nucleotides of the sequence of SEQ ID NO: 1, 3, 97, 98, 99, 100, 101, 102, 103, 104, 105, 106, 107, 109, 110, or 75, 76, 78, 79, 80, 81, 84, 85, 86, wherein the expression repressor is capable of decreasing expression of MYC.

In some aspects, the disclosure provides an expression repressor comprising: a targeting moiety that bind a genomic locus comprising at least 16, 17, 18, 19, or 20 nucleotides of the sequence of SEQ ID NO: 2 or 77, 82, 83 and wherein the expression repressor is capable of decreasing expression of target gene, e.g., MYC. In some embodiments, the expression expressor comprises an effector moiety.

In some aspects, the disclosure provides an expression repressor comprising a targeting moiety wherein the targeting moiety binds a genomic locus that is within 1400 nt upstream or downstream of SEQ ID NO: 4.

In some aspects, the disclosure provides an expression repressor comprising a targeting moiety wherein, the targeting moiety binds a genomic locus comprising at least 14, 15, 16, 17, 18, 19, or 20 nucleotides of the sequence of SEQ ID NO: 4, 77, 82, or 83.

In some aspects, the disclosure provides an expression repressor comprising a targeting moiety wherein, the targeting moiety binds a genomic locus comprising at least 14, 15, 16, 17, 18, 19, or 20 nucleotides of the sequence of SEQ ID NO: 83, 96, or 108.

In some aspects, the disclosure provides a system comprising a first expression repressor comprising a first targeting moiety and optionally a first effector moiety, wherein the first expression repressor binds to a transcription regulatory element (e.g., a promoter or transcription start site (TSS)) operably linked to a target gene, e.g., MYC or to a sequence proximal to the transcription regulatory element, and a second expression repressor comprising a second targeting moiety and optionally a second effector moiety, wherein the second expression repressor binds to an anchor sequence of an anchor sequence mediated conjunction (ASMC) comprising a target gene, e.g., MYC or to a sequence proximal to the anchor sequence.

In some aspects, the disclosure provides a system comprising a first expression repressor comprising a first targeting moiety and optionally a first effector moiety, wherein the first expression repressor binds to a transcription regulatory element (e.g., a promoter or transcription start site (TSS)) operably linked to a target gene, e.g., MYC, or to a sequence proximal to the transcription regulatory element, and a second expression repressor comprising a second targeting moiety and optionally a second effector moiety, wherein the second expression repressor binds to a genomic locus located in a super enhancer region of a target gene, e.g., MYC.

In some embodiments, the first targeting moiety specifically binds a first DNA sequence and the second targeting moiety specifically binds a second DNA sequence different from the first DNA sequence. In some embodiments, the first effector moiety is different from the second effector moiety.

In some aspects, the disclosure provides an expression repressor comprising: a targeting moiety comprising a CRISPR/Cas molecule, e.g., comprising a catalytically inactive CRISPR/Cas protein, that binds to a transcription regulatory element (e.g., a promoter or transcription start site (TSS)) operably linked to a target gene, e.g., MYC or a sequence proximal to said transcription regulatory element; and an effector moiety comprising MQ1 or a functional variant or fragment thereof.

In some aspects, the disclosure provides an expression repressor comprising: a targeting moiety comprising a CRISPR/Cas molecule, e.g., comprising a catalytically inactive CRISPR/Cas protein that binds to a genomic locus located in a super enhancer region of a target gene, e.g., MYC, and an effector moiety comprising KRAB, MQ1, or a functional variant or fragment thereof, wherein the expression repressor is capable of decreasing expression of target gene, e.g., MYC.

In some aspects, the disclosure provides an expression repressor comprising: a targeting moiety comprising a CRISPR/Cas molecule, e.g., comprising a catalytically inactive CRISPR/Cas protein, that binds to an anchor sequence of an anchor sequence mediated conjunction (ASMC) comprising a target gene, e.g., MYC or to a sequence proximal to the anchor sequence; and an effector moiety comprising KRAB or a functional variant or fragment thereof.

In some aspects, the disclosure provides an expression repressor comprising: a targeting moiety comprising a zinc finger molecule that binds to a transcription regulatory element (e.g., a promoter or transcription start site (TSS)) operably linked to a target gene, e.g., MYC or a sequence proximal to said transcription regulatory element; and an effector moiety comprising MQ1 or a functional variant or fragment thereof.

In some aspects, the disclosure provides an expression repressor comprising: a targeting moiety comprising a zinc finger molecule that binds to an anchor sequence of an anchor sequence mediated conjunction (ASMC) comprising a target gene, e.g., MYC or to a sequence proximal to the anchor sequence; and an effector moiety comprising KRAB or a functional variant or fragment thereof.

In some aspects, the disclosure provides an expression repressor comprising: a targeting moiety comprising a zinc finger molecule, that binds to a genomic locus located in a super enhancer region of a target gene, e.g., MYC, and an effector moiety comprising KRAB or a functional variant or fragment thereof.

In some aspects, the disclosure is directed to a nucleic acid encoding the first expression repressor, second expression repressor, both, or a component thereof (e.g., a gRNA, a mRNA). In some embodiments, the nucleic acid encoding the expression repressor system is a multi-cistronic sequence. In some embodiments, the multi-cistronic sequence is a bi-cistronic sequence.

In some aspects, the disclosure is directed to a vector comprising a nucleic acid, a system, or an expression repressor described herein. In another aspect, the disclosure is directed to a lipid nanoparticle comprising a vector, a nucleic acid, a system, or an expression repressor described herein. In another aspect, the disclosure is directed to a reaction mixture comprising an expression repressor, a system, a nucleic acid, a vector, or a lipid nanoparticle described herein. In another aspect, the disclosure is directed to a pharmaceutical composition comprising an expression repressor, a system, a nucleic acid, a vector, a lipid nanoparticle, or a reaction mixture described herein.

In some aspects, the disclosure is directed to a method of decreasing expression of a target gene comprising providing an expression repressor or an expression repression system described herein and contacting the target gene and/or one or more operably linked transcription control elements with the expression repressor or expression repression system, thereby decreasing expression of the target gene.

In some aspects, the disclosure is directed to a method of treating a condition associated with over-expression of a target gene e.g., MYC in a subject, comprising administering an expression repressor, or a system, nucleic acid, or vector described herein to the subject, thereby treating the condition.

In some aspects, the disclosure is directed to a method of treating a condition associated with mis-regulation of a target gene, e.g., MYC, in a subject, comprising administering an expression repressor, system, nucleic acid, or vector described herein to the subject, thereby treating the condition.

In some aspects, the disclosure provides, a method of decreasing expression of a target gene, e.g., MYC in a cell, the method comprising: contacting the cell with a system comprising: a first expression repressor comprising a first targeting moiety and optionally a first effector moiety, wherein the first expression repressor binds to a transcription regulatory element (e.g., a promoter or transcription start site (TSS)) operably linked to a target gene, e.g., MYC, and a second expression repressor comprising a second targeting moiety and optionally a second effector moiety, wherein the second expression repressor binds to an anchor sequence of an anchor sequence mediated conjunction (ASMC) comprising a target gene, e.g., MYC or to a sequence proximal to the anchor sequence thereby decreasing expression of the target gene, e.g., MYC in the cell.

In some aspects, the disclosure provides a method of decreasing expression of a target gene, e.g., MYC, in a cell, the method comprising: contacting the cell with a system comprising: a first expression repressor comprising a first targeting moiety and optionally a first effector moiety, wherein the first expression repressor binds to a transcription regulatory element (e.g., a promoter or transcription start site (TSS)) operably linked to a target gene, e.g., MYC, and a second expression repressor comprising a second targeting moiety and optionally a second effector moiety, wherein the second expression repressor binds to a genomic locus located in a super enhancer region of a target gene, e.g., MYC, thereby decreasing expression of the target gene, e.g., MYC, in the cell.

The present disclosure further provides, in part, a kit comprising: a) a container comprising a composition comprising an expression repressor comprising a targeting moiety that binds to a target gene, promoter, e.g., MYC, and an effector moiety capable of modulating, e.g., decreasing the expression of the target gene, e.g., MYC, and b) a set of instructions comprising at least one method for modulating the expression of a target gene, e.g., MYC within a cell with said composition.

The present disclosure further provides, in part, a kit comprising: a) a container comprising a composition comprising an expression repressor comprising a targeting moiety that binds to a locus located in a super enhancer region of a target gene, e.g., MYC, and an effector moiety capable of modulating, e.g., decreasing the expression of the target gene, e.g., MYC, and b) a set of instructions comprising at least one method for modulating the expression of a target gene, e.g., MYC within a cell with said composition.

In some aspects, the kit comprises a) a container comprising a composition comprising a system comprising two expression repressors, comprising a first expression repressor comprising a first targeting moiety and optionally a first effector moiety, wherein the first expression repressor binds to a transcription regulatory element (e.g., a promoter or transcription start site (TSS)) operably linked to target gene, e.g., MYC or to a sequence proximal to the transcription regulatory element and an expression repressor comprising a second targeting moiety and optionally a second effector moiety, wherein the second expression repressor binds to an anchor sequence of an anchor sequence mediated conjunction (ASMC) comprising target gene, e.g., MYC or to a sequence proximal to the anchor sequence.

In some aspects, the kit comprises a) a container comprising a composition comprising a system comprising two expression repressors, comprising a first expression repressor comprising a first targeting moiety and optionally a first effector moiety, wherein the first expression repressor binds to a transcription regulatory element (e.g., a promoter or transcription start site (TSS)) operably linked to target gene, e.g., MYC, or to a sequence proximal to the transcription regulatory element and an expression repressor comprising a second targeting moiety and optionally a second effector moiety, wherein the second expression repressor binds to a genomic locus located in a super enhancer region of a target gene, e.g., MYC.

In some embodiments the kit further comprises b) a set of instructions comprising at least one method for treating a disease or modulating, e.g., decreasing the expression of target gene, e.g., MYC within a cell with said composition. In some embodiments, the kits can optionally include a delivery vehicle for said composition (e.g., a lipid nanoparticle). The reagents may be provided suspended in the excipient and/or delivery vehicle or may be provided as a separate component which can be later combined with the excipient and/or delivery vehicle. In some embodiments, the kits may optionally contain additional therapeutics to be co-administered with the compositions to affect the desired target gene expression, e.g., MYC gene expression modulation. While the instructional materials typically comprise written or printed materials, they are not limited to such. Any medium capable of storing such instructions and communicating them to an end user is contemplated by this invention. Such media include but are not limited to electronic storage media (e.g., magnetic discs, tapes, cartridges, chips), optical media (e.g., CD ROM), and the like. Such media may include addresses to internet sites that provide such instructional materials.

Additional features of any of the aforesaid methods or compositions include one or more of the following enumerated embodiments.

Those skilled in the art will recognize or be able to ascertain using no more than routine experimentation, many equivalents to the specific embodiments of the disclosure described herein. Such equivalents are intended to be encompassed by the following enumerated embodiments.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Dec. 15, 2020. When one gene or protein references a plurality of sequence accession numbers, all of the sequence variants are encompassed.

### ENUMERATED EMBODIMENTS

1. An expression repressor comprising:
a targeting moiety that binds to a MYC promoter, and
optionally, an effector moiety,
wherein the expression repressor is capable of decreasing expression of MYC.
   2. The expression repressor of embodiment 1, wherein the targeting moiety binds a genomic locus that is within 1400, 1200, 1000, 800, 600, 400, or 200 nt upstream or downstream of SEQ ID NO: 4, 199, or 201.
   3. The expression repressor of embodiment 1, wherein the targeting moiety binds a genomic locus comprising at least 14, 15, 16, 17, 18, 19, or 20 nucleotides of the sequence of SEQ ID NO: 4, 77, 82, 83, 85, 199, or 201.
   4. An expression repressor comprising:
a targeting moiety that binds a genomic locus comprising at least 14, 15, 16, 17, 18, 19, or 20 nucleotides of the sequence of SEQ ID NO: 3, 97, 98, 99, 100, 101, 102, 103, 104, 105, 106, 107, 109, 110, 75, 76, 78, 79, 80, 81, 84, 85, 86, 190, 191, 192, 200, or 202 and
optionally, an effector moiety,
wherein the expression repressor is capable of decreasing expression of MYC.
   5. An expression repressor comprising:
a targeting moiety that binds a genomic locus comprising at least 16, 17, 18, 19, or 20 nucleotides of the sequence of SEQ ID NO: 2, 77, 82, 83, 199, or 201 and
optionally, an effector moiety,
wherein the expression repressor is capable of decreasing expression of MYC.
   6. An expression repressor comprising:
a targeting moiety that binds a MYC locus, and
an effector moiety comprising MQ1 or a fragment or variant thereof,
wherein the expression repressor is capable of decreasing expression of MYC.
   7. An expression repressor comprising:
a targeting moiety that binds a locus in MYC super enhancer region,
optionally an effector moiety, e.g., an effector moiety comprising a DNA methyltransferase, wherein optionally the effector moiety comprises MQ1 or a fragment or variant thereof,
wherein the expression repressor is capable of decreasing expression of MYC.
   8. An expression repressor comprising:
a targeting moiety that binds a locus in MYC super enhancer region,
an effector moiety comprising a transcription repressor, wherein optionally the effector moiety comprises KRAB or a fragment or variant thereof,
wherein the expression repressor is capable of decreasing expression of MYC.
   9. The expression repressor of embodiment 7 or 8, wherein the targeting moiety binds a genomic locus comprising at least 14, 15, 16, 17, 18, 19, or 20 nucleotides of the sequence of any of SEQ ID NO: 96-110, 83, 199, 201.
   10. The expression repressor of any of embodiments 7-9, wherein the targeting moiety binds a genomic locus comprising at least 14, 15, 16, 17, 18, 19, or 20 nucleotides of the sequence of GRCh37: chr8:129162465-129212140, using the hg19 reference genome.
   11. The expression repressor of any of embodiments 7-10, wherein the targeting moiety binds a genomic locus comprising at least 14, 15, 16, 17, 18, 19, or 20 nucleotides of the sequence of SEQ ID NO: 96 or 108.
   12. The expression repressor of any of embodiments 7-11, wherein the targeting moiety comprises a zinc finger domain or a TAL effector domain.
   13. An expression repressor comprising:
a targeting moiety that binds a locus, e.g., a MYC locus,
a first effector moiety comprising EZH2 or a fragment or variant thereof, and
a second effector moiety comprising KRAB or a fragment or variant thereof,
wherein the expression repressor is capable of decreasing expression at the locus, e.g., decreasing expression of MYC.
   14. The expression repressor of embodiment 13, wherein the targeting moiety binds the MYC promoter, super enhancer region, or anchor sequence.
   15. The expression repressor of embodiment 13 or 14, wherein the targeting moiety comprises a TAL effector domain, a CRISPR/Cas domain, or a zinc finger domain.
   16. The expression repressor of any of embodiments 13-15, wherein the first effector moiety is N-terminal of the second effector, or wherein the first effector is C-terminal of the second effector moiety.
   17. An expression repressor comprising:
a targeting moiety that binds a MYC locus, wherein the targeting moiety comprises a zinc finger domain, and
optionally, an effector moiety,
wherein the expression repressor is capable of decreasing expression of MYC.
   18. An expression repressor comprising:
a targeting moiety comprising a CRISPR/Cas domain, e.g., comprising a catalytically inactive CRISPR/Cas protein, that binds to a transcription regulatory element (e.g., a promoter, an enhancer, a super enhancer, or transcription start site (TSS)) operably linked to a MYC gene or a sequence proximal to said transcription regulatory element; and
an effector moiety comprising MQ1 or a functional variant or fragment thereof.
   19. An expression repressor comprising:
a targeting moiety comprising a CRISPR/Cas domain, e.g., comprising a catalytically inactive CRISPR/Cas protein, that binds to a transcription regulatory element (e.g., a promoter, an enhancer, or transcription start site (TSS)) operably linked to a MYC gene or a sequence proximal to said transcription regulatory element; and
an effector moiety comprising MQ1 or a functional variant or fragment thereof.
   20. An expression repressor comprising:
a targeting moiety comprising a CRISPR/Cas domain, e.g., comprising a catalytically inactive CRISPR/Cas protein, that binds to a transcription regulatory element (e.g., a promoter, an enhancer, or transcription start site (TSS)) operably linked to a MYC gene or a sequence proximal to said transcription regulatory element; and
an effector moiety comprising KRAB or a functional variant or fragment thereof.
   21. An expression repressor comprising:
a targeting moiety comprising a CRISPR/Cas domain, e.g., comprising a catalytically inactive CRISPR/Cas protein, that binds to an anchor sequence of an anchor sequence mediated conjunction (ASMC) comprising a MYC gene or to a sequence proximal to the anchor sequence; and
an effector moiety comprising KRAB or a functional variant or fragment thereof.
   22. An expression repressor comprising:
a targeting moiety comprising a zinc finger domain that binds to a transcription regulatory element (e.g., a promoter, an enhancer, or transcription start site (TSS)) operably linked to a MYC gene or a sequence proximal to said transcription regulatory element; and
an effector moiety comprising MQ1 or a functional variant or fragment thereof.
   23. An expression repressor comprising:
a targeting moiety comprising a zinc finger domain that binds to a transcription regulatory element (e.g., a promoter, an enhancer, or transcription start site (TSS)) operably linked to a MYC gene or a sequence proximal to said transcription regulatory element; and
an effector moiety comprising KRAB or a functional variant or fragment thereof.
   24. An expression repressor comprising:
a targeting moiety that binds a mouse genomic locus comprising at least 14, 15, 16, 17, 18, 19, or nucleotides of the sequence of any of SEQ ID NOs: 190-192 and
optionally, an effector moiety,
   wherein the expression repressor is capable of decreasing expression of MYC.
   25. The expression repressor of claim 24, wherein the effector moiety comprises a DNA methyltransferase, e.g., MQ1 or a fragment or variant thereof.
   26. The expression repressor of embodiments 24 or 25, wherein the targeting moiety comprises a TAL effector domain, a CRISPR/Cas domain, a zinc finger domain, a tetR domain, a meganuclease domain, or an oligonucleotide.
   27. The expression repressor of any of embodiments 24-26, wherein the targeting moiety comprises a zinc finger domain or a TAL effector domain.
   28. The expression repressor of any of embodiments 24-27, wherein the expression repressor comprises an amino acid sequence chosen from any of SEQ ID NOs: 160-165, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
   29. The expression repressor of any of embodiments 24-28, wherein the expression repressor is encoded by a nucleotide sequence chosen from any of SEQ ID NOs: 166-168, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
   30. The expression repressor of any of embodiments 24-29, wherein the targeting moiety comprises an amino acid sequence according to any of SEQ ID NOs: 154-156, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
   31. The expression repressor of any of embodiments 24-30, wherein the targeting moiety comprises a nucleic acid sequence according to any of SEQ ID NOs: 157-159, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
   32. The expression repressor of any of embodiments 24-31, wherein the effector moiety is a durable effector moiety.
   33. The expression repressor of any of embodiments 24-32, wherein the effector moiety is a transient effector moiety.
   34. The expression repressor of any of embodiments 24-33, wherein the expression repressor is a fusion molecule.
   35. The expression repressor of any of embodiments 24-34, wherein the targeting moiety comprises a zinc finger domain, and the effector moiety comprises an epigenetic modifying moiety, e.g., a DNA methyltransferase, e.g., MQ1 or a fragment or variant thereof.
   36. The expression repressor of any of embodiments 18-20, 22, or 23, wherein the regulatory element is part of a cluster of regulatory elements.
   37. The expression repressor of any embodiments 18-20, 22, or 23, wherein the regulatory element is located in a non-coding region.
   38. The expression repressor of any embodiments 18-20, 22, or 23, wherein the regulatory element is a distal enhancer e.g., located at least 1,000 nt away from a target gene promoter, e.g., MYC.
   39. The expression repressor of any embodiments 18-20, 22, 23 or 36-38, wherein the regulatory element increases the expression of a target gene, e.g., MYC.
   40. The expression repressor of any embodiments 18-20, 22, 23, or 36-39, wherein the regulatory element contains one or more mutations.
   41. The expression repressor of any embodiments 18-20, 22, 23, or 36-40, wherein the regulatory element contains at least one disease-associated single nucleotide polymorphism (SNP).
   42. The expression repressor of any of embodiments 18-20, 22, 23, or 36-41, wherein the transcription regulatory element interacts with the promoter of target gene, e.g., MYC through an enhancer docking site.
   43. The expression repressor of embodiment 42, wherein the enhancer docking site comprises a nucleotide sequence of according to any of SEQ ID NOs: 71-74.
   44. An expression repressor comprising:
a targeting moiety comprising a zinc finger domain that binds to an anchor sequence of an anchor sequence mediated conjunction (ASMC) comprising a MYC gene or to a sequence proximal to the anchor sequence; and
an effector moiety comprising KRAB or a functional variant or fragment thereof.
   45. The expression repressor of any of embodiments 1-23 or 36-43, wherein the expression repressor comprises an amino acid sequence chosen from any of SEQ ID NOs: 22-37, 129, 133, 134, 139-149, or 177-186, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
   46. The expression repressor of any of embodiments 1-23 or 36-45, wherein the expression repressor is encoded by a nucleotide sequence chosen from any of SEQ ID NOs: 55-70, 130, 189, or 193-197, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
   47. The expression repressor of any of embodiments 1-23 or 36-46, wherein the targeting moiety comprises an amino acid sequence according to any of SEQ ID NOs: 5-16, or 169-172, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
   48. The expression repressor of any of the preceding embodiments, wherein the effector moiety comprises an amino acid sequence according to SEQ ID NO: 18 19, or 87, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto
   49. The expression repressor of any of embodiments 1-12, 17-19, 22, 36-42, or 44-47, wherein the effector moiety is a durable effector moiety.
   50. The expression repressor of any of embodiments 1-23, or 36-48 wherein the effector moiety is a transient effector moiety.
   51. The expression repressor of any of embodiments 1-12, 17-19, 22, 36-42, or 44-48, wherein the effector moiety comprises a DNA methyltransferase, e.g., MQ1 or a fragment or variant thereof.
   52. The expression repressor of any of embodiments 1-23, 36-47 or 49, wherein the effector moiety comprises a transcription repressor, e.g., comprises KRAB or a fragment or variant thereof.
   53. The expression repressor of any of the preceding embodiments, wherein the targeting moiety comprises a TAL effector domain, a CRISPR/Cas domain, a zinc finger domain, a tetR domain, a meganuclease domain, or an oligonucleotide.
   54. The expression repressor of embodiment 53, wherein the CRISPR/Cas domain binds a gRNA, e.g., a gRNA that binds a genomic locus comprising at least 14, 15, 16, 17, 18, 19, or 20 nucleotides of the sequence of any of SEQ ID NOs: 1-4, e.g., wherein the gRNA comprises a sequence that comprises at least 14, 15, 16, 17, 18, 19, or 20 nucleotides of the sequence of any of SEQ ID NOs: 1-4.
   55. The expression repressor of embodiment 53, wherein the CRISPR/Cas domain binds a gRNA, e.g., a gRNA that binds a genomic locus comprising at least 14, 15, 16, 17, 18, 19, or 20 nucleotides of the sequence of any of SEQ ID NOs: 96-110, e.g., wherein the gRNA comprises a sequence that comprises at least 14, 15, 16, 17, 18, 19, or 20 nucleotides of the sequence of any of SEQ ID NOs: 96-110.
   56. The expression repressor of any of embodiments 53-55, wherein the CRISPR/Cas domain comprises a Cas protein or Cpf1 protein chosen from Table 1 or a variant (e.g., mutant) of any thereof.
   57. The expression repressor of any of embodiments 53-56, wherein the CRISPR/Cas domain comprises a catalytically inactive CRISPR/Cas protein, e.g., dCas9.
   58. The expression repressor of embodiment 53, wherein the zinc finger domain binds a genomic locus comprising at least 14, 15, 16, 17, 18, 19, or 20 nucleotides of the sequence of any of SEQ ID NOs: 96-110, e.g., wherein the gRNA comprises a sequence that comprises at least 14, 15, 16, 17, 18, 19, or 20 nucleotides of the sequence of any of SEQ ID NOs: 96-110.
   59. The expression repressor of any of embodiments 17, 22, 26-53, or 57, wherein the zinc finger domain comprises 1, 2, 3, 4, 5, 6, 7, 8, 9, or 10 zinc fingers (and optionally no more than 11, 10, 9, 8, 7, 6, or 5 zinc fingers).
   60. The expression repressor of any of embodiments 17, 22, 26-53, 57, or 58, wherein the zinc finger domain comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6- 7, 7-10, 7-9, 7-8, 8-10, 8-9, or 9-10 zinc fingers.
   61. The expression repressor of any of embodiments 17, 22, 26-53, or 57-59, wherein the zinc finger domain comprises 3 or 9 zinc fingers.
   62. The expression repressor of any of the preceding embodiments, which is a fusion molecule.
   63. The expression repressor of any of the preceding embodiments, which comprises a linker situated between the targeting domain and the effector domain, optionally wherein the linker comprises an amino sequence according to SEQ ID NO: 137 or SEQ ID NO: 138.
   64. The expression repressor of any of embodiments 1-17, 20, 21, 23, 44-48, 50, or 52-57, wherein the targeting moiety comprises a catalytically inactive CRISPR/Cas domain (e.g., dCas9) and the effector moiety comprises a transcription repressor, e.g., KRAB or a fragment or variant thereof.
   65. The expression repressor of any of embodiments 1-17, 20, 21, 23, 44-48, 50, 52, or 53-64, wherein the targeting moiety comprises a zinc finger domain, and the effector moiety comprises a transcription repressor, e.g., KRAB or a fragment or variant thereof.
   66. The expression repressor of any of embodiments 17, 36-43, 45-47, 53, or 58-63, wherein the targeting moiety comprises a zinc finger domain, and the expression repressor does not comprise an effector moiety.
   67. The expression repressor of any of embodiments 1-12, 18-19, 22, 36-43, 45-49, 51, or 53-57 wherein the targeting moiety comprises a catalytically inactive CRISPR/Cas domain (e.g., dCas9) and the effector moiety comprises an epigenetic modifying moiety, e.g., a DNA methyltransferase, e.g., MQ1 or a fragment or variant thereof.
   68. The expression repressor of any of embodiments 1-12, 17-19, 22, 36-43, 45-49, 51, 53, or 58-63, wherein the targeting moiety comprises a zinc finger domain, and the effector moiety comprises an epigenetic modifying moiety, e.g., a DNA methyltransferase, e.g., MQ1 or a fragment or variant thereof.
   69. The expression repressor of any of the preceding embodiments, which comprises an amino acid sequence of any of SEQ ID NOS: 22-37, 129, 133, 134, 139-149, or 177-186, or a sequence having at least. 80%, 85%, 90%, 95%, 96%, 97%, 98%, 99% identity thereto.
   70. The expression repressor of any of the preceding embodiments, which: (i) comprises one or more nuclear localization signal sequences (NLS), or (ii) does not comprise an NLS.
   71. The expression repressor of any of the preceding embodiments, comprising a first NLS at the N terminus, e.g., wherein the first NLS has a sequence of SEQ ID NO: 88.
   72. The expression repressor of any of the preceding embodiments, comprising an NLS, e.g., a second NLS, at the C terminus, e.g., having a sequence of SEQ ID NO: 89.
   73. The expression repressor of any of the preceding embodiments, wherein the first and the second NLS have the same sequence.
   74. The expression repressor of any of embodiments 71-73, wherein the first and the second NLS have different sequences.
   75. The expression repressor of any of the preceding embodiments, which comprises an epitope tag.
   76. The expression repressor of embodiment 75, wherein the epitope tag is an HA tag.
   77. The expression repressor of any of preceding embodiments, wherein the anchor sequence comprises the sequence of SEQ ID NO: 71 or 72, or a sequence with no more than 8, 7, 6, 5, 4, 3, 2, or 1 alterations relative thereto.
   78. The expression repressor of any of embodiments 1-77, wherein the anchor sequence comprises a sequence according to SEQ ID NO: 73 or 74, or a sequence with no more than 8, 7, 6, 5, 4, 3, 2, or 1 alterations relative thereto.
   79. The expression repressor of any of preceding embodiments, wherein the anchor sequence is on the same chromosome as the MYC gene.
   80. The expression repressor of any of preceding embodiments, wherein the anchor sequence is upstream of the MYC gene (e.g., upstream of the TSS or upstream of the promoter).
   81. The expression repressor of any of preceding embodiments, wherein the anchor sequence is at least 1, 5, 10, 50, 100, or 1000 kilobases away from the MYC gene (e.g., from the TSS or promoter of the MYC gene).
   82. The expression repressor of any of preceding embodiments, wherein the anchor sequence is 0.1-0.5, 0.1-1, 0.1-5, 0.1-10, 0.1-50, 0.1-100, 0.1-500, 0.1-1000, 0.5-1, 0.5-5, 0.5-10, 0.5-50, 0.5-100, 0.5-500, 0.5-1000, 1-5, 1-10, 1-50, 1-100, 1-500, 1-1000, 5-10, 5-50, 5-100, 5-500, 5-1000, 10-50, 10-100, 10-500, 10-1000, 50-100, 50-500, 50-1000, 100-500, 100-1000, or 500-1000 kilobases away from the MYC gene (e.g., from the TSS or promoter of the MYC gene).
   83. The expression repressor of any of embodiments 1-79 or 81-82, wherein the target sequence is downstream of the MYC gene (e.g., downstream of the TSS or downstream of the promoter).
   84. The expression repressor of any of preceding embodiments, wherein the targeting moiety binds to a sequence at chromosome coordinates 128746342-128746364, 128746321-128746343, 128746525-128746547, 128748014-128748036, 129188878-129188900, 129188958-129188980, 129188960-129188982, 129189067-129189089, 129189457-129189479, 129189554-129189576, 129189679-129189701, 129209511-129209533, 129209643-129209665, 129209658-129209680, 129209856-129209878, 129189452-129189474, 129189190-129189212, 129189274-129189296, 129189421-129189443, 128746405-128746425, 128748069-128748089, 129188825-129188845, or 129188822-129188842 or a sequence proximal thereto.
   85. The expression repressor of any of the preceding embodiments, wherein binding of the expression repressor to the target gene locus, e.g., MYC, increases methylation at a site in the target gene locus, e.g., MYC, by 10, 20, 30, 40, 50, 60, 70, 80, 90, or 100% compared to methylation in the absence of the expression repressor, e.g., as measured by ELISA or as described in any of Examples 7 or 28, wherein optionally the site assayed for methylation is chr8:129188693-129189048 according to hg19 reference genome, e.g., comprises a sequence according to SEQ ID NO: 123.
   86. The expression repressor of any of the preceding embodiments, wherein binding of the expression repressor to the target gene locus, e.g., MYC increases methylation at a site in the target gene locus, e.g., MYC for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as described in Example 28.
   87. The expression repressor of any of the preceding embodiments, wherein binding of the expression repressor to the MYC locus decreases expression of MYC in a cell by 10, 20, 30, 40, 50, 60, 70, 80, 90, or 100% compared to expression in the absence of the expression repressor, e.g., as measured by ELISA or as described in any of Examples 2-7 or 9.
   88. The expression repressor of any of the preceding embodiments, wherein binding of the expression repressor to the MYC locus appreciably decreases expression of MYC for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA or as described in any of Examples 2-7 or 9.
   89. The expression repressor of any of the preceding embodiments, wherein binding of the expression repressor to the MYC locus appreciably decreases expression of MYC at 1, 2, 3, 4, 5, 6, 7, 8, 10, 12, 16, 20, 24, 28, 32, 36, 40, 44, 48, 52, 56, 60, 64, 68, 72, 76, 80, or 96 hours post-transfection.
   90. The expression repressor of any of embodiments 1-23 or 36-89, wherein the targeting moiety binds to a human genomic locus.
   91. The expression repressor of any of embodiments 24-43 49, 51, 53, 56-57, 59-62, 66-68, 70-89, wherein the targeting moiety binds to a mouse genomic locus.
   92. The expression repressor of any of the preceding embodiments, wherein binding of the expression repressor to the MYC locus decreases the viability of a cell comprising the MYC locus (e.g., cancer cells).
   93. The expression repressor of any of the preceding embodiments, wherein contacting a plurality of cells with the expression repressor or a nucleic acid encoding the expression repressor decreases the viability of the plurality of cells.
   94. The expression repressor of any of the preceding embodiments, wherein viability is decreased by 10, 20, 30, 40, 50, 60, 70, 80, 90, or 100% compared to viability in the absence of the first expression repressor, e.g., as measured by CellTiter Glo or as described in any of Examples 2-7.
   95. The expression repressor of any of the preceding embodiments, wherein administration of the expression repressor results in apoptosis of at least 5%, 6%, 7%, 8%, 9% 10%, 12%, 15%, 17% 20%, 25% 30%, 40%, 45%, 50%, 55%, 60%, 65%, 75% of target cells (e.g., cancer cells).
   96. The expression repressor of any preceding embodiments, wherein the plurality of cells comprises a plurality of cancer cells and a plurality of non-cancer cells and/or a plurality of infected cells and a plurality of uninfected cells.
   97. The expression repressor of any of the preceding embodiments, wherein contacting the plurality of cells with the expression repressor or a nucleic acid encoding the expression repressor decreases the viability of the plurality of cancer cells more than it decreases the viability of the plurality of non-cancer cells.
   98. The expression repressor of any of the preceding embodiments, wherein contacting the plurality of cells with the expression repressor or a nucleic acid encoding the expression repressor decreases the viability of the plurality of cancer cells 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than it decreases the viability of the plurality of non-cancer cells.
   99. The expression repressor of any of embodiments 92-97, wherein the cancer cells are lung cancer cells, gastric cancer cells, gastrointestinal cancer cells, colorectal cancer cells, pancreatic cancer cells, or hepatic cancer cells.
   100. The expression repressor of any of embodiments 92-99, wherein the cancer is hepatocellular carcinoma (HCC), fibrolamellar hepatocellular carcinoma (FHCC), cholangiocarcinoma, angiosarcoma, secondary liver cancer, non-small cell lung cancer (NSCLC), adenocarcinoma, small cell lung cancer (SCLC), large cell (undifferentiated) carcinoma, triple negative breast cancer, gastric adenocarcinoma, endometrial carcinoma, or pancreatic carcinoma.
   101. The expression repressor of any of the preceding embodiments, which, when contacted with a plurality of infected cells and a plurality of uninfected cells, decreases the viability of the plurality of infected cells more than it decreases the viability of the plurality of uninfected cells.
   102. The expression repressor of any of preceding embodiments, wherein the infection is viral.
   103. The expression repressor of embodiment 102, wherein the viral infection is hepatitis, e.g., hepatitis B.
   104. The expression repressor of any of embodiments 92-103, wherein the infected cells are human hepatocytes.
   105. The expression, repressor of any of the preceding embodiments, which has an EC50 of 0.04-0.4, 0.04-0.1, 0.1-0.2, 0.2-0.3, or 0.3-0.4 μg/mL when tested in an assay for viability of cancer cells (e.g., HCC cells) using LNP delivery of mRNA encoding the expression repressor, e.g., in an assay according to Example 12.
   106. The expression repressor of any of embodiments 1-104, which has an EC50 of 0.1-2.5, 0.5-2.2, 1.0-1.5, 1.2-2 μg/mL when tested in an assay for viability of cancer cells (e.g., lung cancer cells) using LNP delivery of mRNA encoding the expression repressor, e.g., in an assay according to Example 18.
   107. The expression repressor of any of the preceding embodiments, which has an EC50 of 0.004-0.08, 0.004-0.01, 0.01-0.02, 0.02-0.04, or 0.04-0.08 μg/mL when tested in an assay for reducing MYC mRNA levels in cancer cells (e.g., HCC cells) using LNP delivery of mRNA encoding the expression repressor, e.g., in an assay according to Example 12.
   108. The expression repressor of any of the preceding embodiments, which has an EC50 of 0.04-0.1, 0.04-0.09, 0.05-0.09, or 0.06-0.8 μg/mL when tested in an assay for reducing MYC mRNA levels in cancer cells (e.g., lung cancer cells) using LNP delivery of mRNA encoding the expression repressor, e.g., in an assay according to Example 18.
   109. The expression repressor of any of the preceding embodiments, which reduces the level of a protein encoded by a target gene, e.g., MYC in a cell by at least 10%, at least 20%, at least 30%, at least 40%, at least 50%, at least 60%, at least 70%, at least 80%, or at least 90% compared to the protein level in an untreated cell.
   110. The expression repressor of any of the preceding embodiments, which is capable of reducing tumor volume, e.g., in a human subject or in a mammalian model.
   111. The expression repressor of any of preceding embodiments, wherein the expression repressor is capable of reducing tumor volume to a similar or greater degree compared to a chemotherapeutic agent, e.g., in a mammalian model, e.g., when measured at day 20 after initiation of treatment, e.g., wherein the expression repressor is administered every 5 days at a dose of 3 mg/kg.
   112. The expression repressor of any of preceding embodiments, wherein the expression repressor is capable of reducing tumor volume compared to a PBS control, e.g., in a mammalian model, e.g., when measured at day 20 after initiation of treatment e.g., wherein the expression repressor is administered every 5 days for 4 doses followed by every 3 days for 3 doses at 1 mg/kg, 1.5 mg/kg, or 3 mg/kg.
   113. The expression repressor of any of preceding embodiments, wherein the tumor volume is reduced by at least about 10%, 20%, 30%, or 40% compared to a control treated with PBS, e.g., at day 20 after start of treatment.
   114. The expression repressor of any of embodiment 111-113, wherein the chemotherapeutic agent is sorafenib or cisplatin.
   115. The expression repressor of any of preceding embodiments, wherein the system is capable of reducing tumor volume to a similar or greater degree compared to a small molecule MYC inhibitor.
   116. The expression repressor of embodiment 115, wherein the small molecule MYC inhibitor is MYCi975 wherein optionally tumor volume is reduced by at least about 10%, 20%, 30%, or 40% compared to a control treated with the MYCi975, e.g., at day 20 after start of treatment.
   117. The expression repressor of any of preceding embodiments, which does not cause a decrease in body weight compared to at the start of treatment, or which causes a decrease in body weight of less than 3%, 2%, or 1%.
   118. A system comprising:
a first expression repressor of any of the preceding embodiments, and
a second expression repressor, e.g., a second expression repressor described herein, e.g., a second expression repressor of any of the preceding embodiments.
   119. A system comprising:
a first expression repressor comprising a first targeting moiety and optionally a first effector moiety, wherein the first expression repressor binds to a transcription regulatory element (e.g., a promoter, enhancer, or transcription start site (TSS)) operably linked to a MYC gene or to a sequence proximal to the transcription regulatory element, and
a second expression repressor comprising a second targeting moiety and optionally a second effector moiety, wherein the second expression repressor binds to an anchor sequence of an anchor sequence mediated conjunction (ASMC) comprising a MYC gene or to a sequence proximal to the anchor sequence.
   120. The system of embodiment 118 or 119,
wherein the transcription regulatory element comprises a promoter, and
wherein the anchor sequence comprises a CTCF binding motif.
   121. The system of any of embodiments 118-120, wherein second expression repressor binds to a downstream region adjacent to the CTCF binding motif.
   122. The system of any of embodiments 118-120, wherein second expression repressor binds to an upstream region adjacent to the CTCF binding motif.
   123. The system of any of embodiments 118-122, wherein
the first expression repressor comprises a targeting moiety that binds a genomic locus comprising at least 16, 17, 18, 19, or 20 nucleotides of the sequence of SEQ ID NO: 2, 3, 4, 71-86, or 200-206; and
the second expression repressor comprises a targeting moiety that binds a genomic locus comprising at least 16, 17, 18, 19, or 20 nucleotides of the sequence of SEQ ID NO: 2, 3, 4, 71-86, or 200-206.
   124. The system of any of embodiments 118-123, wherein
the first expression repressor comprises a targeting moiety that binds a genomic locus comprising at least 16, 17, 18, 19, or 20 nucleotides of the sequence of any of SEQ ID NO: 96-110.
   125. The system of any of embodiments 118-124, wherein,
the first expression repressor comprises a targeting moiety that binds a genomic locus comprising at least 16, 17, 18, 19, or 20 nucleotides of the sequence of SEQ ID NO: 71, SEQ ID NO: 72, or SEQ ID NO: 83; and
the second expression repressor comprises a targeting moiety that binds a genomic locus comprising at least 16, 17, 18, 19, or 20 nucleotides of the sequence of SEQ ID NO: 77.
   126. A system comprising:
a first expression repressor comprising a first targeting moiety and optionally a first effector moiety, wherein the first expression repressor binds to a promoter operably linked to a MYC gene or to a sequence proximal to the promoter, and
a second expression repressor comprising a second targeting moiety and optionally a second effector moiety, wherein the second expression repressor binds to an enhancer (e.g., a super-enhancer) of the MYC gene.
   127. The system of embodiment 126, wherein,
the first expression repressor comprises a targeting moiety that binds a genomic locus comprising at least 16, 17, 18, 19, or 20 nucleotides of the sequence of SEQ ID NO: 204, and
the second expression repressor comprises a targeting moiety that binds a genomic locus comprising at least 16, 17, 18, 19, or 20 nucleotides of the sequence of any of SEQ ID NOs: 199 or 201.
   128. A system for reducing MYC expression, the system comprising:
a) a first expression repressor comprising:
   i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto and
   ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second expression repressor comprising:
   i) a second targeting moiety having an amino acid sequence according to SEQ ID NO: 7 169, or 171 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   ii) a second effector moiety having an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      129. The system of embodiments 128, wherein the first expression repressor further comprises a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the first targeting moiety.
      130. The system of embodiment 128 or 129, wherein the first expression repressor further comprises a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the first effector moiety.
      131. The system of any of embodiments 128-130, wherein the second expression repressor further comprises a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the second targeting moiety.
      132. The system of any of embodiments 128-131, wherein the second expression repressor further comprises a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the second effector moiety.
      133. The system of any of embodiments 128-132, wherein the first expression repressor further comprises a first linker situated between the first targeting moiety and the first effector moiety, wherein optionally the first linker has an amino acid sequence according to SEQ ID NO: 137 or a sequence with at least 80, 85, 90, 95, 99, or 100% identity thereto.
      134. The system of any of embodiments 128-133, wherein the second expression repressor further comprises a second linker situated between the second targeting moiety and the second effector moiety, wherein optionally the second linker has an amino acid sequence according to SEQ ID NO: 138 or a sequence with at least 80, 85, 90, 95, 99, or 100% identity thereto.
      135. The system of any of embodiments 128-134, wherein the first expression repressor further comprises an amino acid sequence C-terminal of the first effector moiety, e.g., a sequence of up to 30, 25, 20, or 18 amino acids, e.g., a sequence according to SEQ ID NO: 126 or a sequence with at least 80, 85, 90, 95, 99, or 100% identity thereto.
      136. The system of any of embodiments 128-132, wherein the second expression repressor further comprises an amino acid sequence N-terminal of the second targeting moiety, e.g., a sequence of up to 30, 25, 20, or 18 amino acids, e.g., a sequence according to SEQ ID NO: 128 or a sequence with at least 80, 85, 90, 95, 99, or 100% identity thereto.
      137. The system of any of embodiments 128-136, wherein the first expression repressor has an amino acid sequence according to SEQ ID NO: 30 or 129, or a sequence with at least 80, 85, 90, 95, 99, or 100% identity thereto.
      138. The system of any of embodiments 128-137, wherein the second expression repressor has an amino acid sequence according to SEQ ID NO: 24, or a sequence with at least 80, 85, 90, 95, 99, or 100% identity thereto.
      139. The system of any of embodiments 128-137, wherein the second targeting moiety comprises an amino acid sequence according to SEQ ID NO: 169, or a sequence with at least 80, 85, 90, 95, 99, or 100% identity thereto.
      140. The system of any of embodiments 128-137, wherein the second targeting moiety comprises an amino acid sequence according to SEQ ID NO: 171, or a sequence with at least 80, 85, 90, 95, 99, or 100% identity thereto.
      141. The system of any of embodiments 128-140, wherein the second expression repressor has an amino acid sequence according to SEQ ID NO: 177 or 183 or a sequence with at least 80, 85, 90, 95, 99, or 100% identity thereto.
      142. The system of any of embodiments 128-140, wherein the second expression repressor has an amino acid sequence according to SEQ ID NO: 179, 185, or a sequence with at least 80, 85, 90, 95, 99, or 100% identity thereto.
      143. A nucleic acid encoding the first expression repressor and second repressor of the system of any of embodiments 128-142.
      144. A nucleic acid encoding a system for reducing MYC expression, the nucleic acid comprising:
a) a first region encoding a first expression repressor, the first expression repressor comprising:
   i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second region encoding a second expression repressor, the second expression repressor comprising:
   i) a second targeting moiety having an amino acid sequence according to SEQ ID NO: 7 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   ii) a second effector moiety having an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      145. The nucleic acid of embodiment 144, wherein the first region is 5′ of the second region.
      146. The nucleic acid of embodiment 144, wherein the first region is 3′ of the second region.
      147. The nucleic acid of embodiment 145 or 146, wherein the first region further comprises a nucleotide sequence encoding a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the first targeting moiety.
      148. The nucleic acid of any of embodiments 145-147, wherein the first region further comprises a nucleotide sequence encoding a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the first effector moiety.
      149. The nucleic acid of any of embodiments 145-148, wherein the second region further comprises a nucleotide sequence encoding a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the second targeting moiety.
      150. The nucleic acid of any of embodiments 145-149, wherein the second region further comprises a nucleotide sequence encoding a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the second effector moiety.
      151. The nucleic acid of any of embodiments 145-150, wherein the first region further comprises a nucleotide sequence encoding a first linker situated between the first targeting moiety and the first effector moiety, wherein optionally the first linker has an amino acid sequence according to SEQ ID NO: 137 or a sequence with at least 80, 85, 90, 95, 99, or 100% identity thereto.
      152. The nucleic acid of any of embodiments 145-151, wherein the second region further comprises a nucleotide sequence encoding a second linker situated between the second targeting moiety and the second effector moiety, wherein optionally the second linker has an amino acid sequence according to SEQ ID NO: 138 or a sequence with at least 80, 85, 90, 95, 99, or 100% identity thereto.
      153. The nucleic acid of any of embodiments 145-152, wherein the first region further comprises a nucleotide sequence encoding an amino acid sequence C-terminal of the first effector moiety, e.g., a sequence of up to 30, 25, 20, or 18 amino acids, e.g., a sequence according to SEQ ID NO: 126 or a sequence with at least 80, 85, 90, 95, 99, or 100% identity thereto.
      154. The nucleic acid of any of embodiments 145-153, wherein the second region further comprises a nucleotide sequence encoding an amino acid sequence N-terminal of the second targeting moiety, e.g., a sequence of up to 30, 25, 20, or 18 amino acids, e.g., a sequence according to SEQ ID NO: 128 or a sequence with at least 80, 85, 90, 95, 99, or 100% identity thereto.
      155. The nucleic acid of any of embodiments 145-154, wherein the first expression repressor has an amino acid sequence according SEQ ID NO: 30 or 129, or a sequence with at least 80, 85, 90, 95, 99, or 100% identity thereto.
      156. The nucleic acid of any of embodiments 145-155, wherein the second expression repressor has an amino acid sequence according to SEQ ID NO: 24, or a sequence with at least 80, 85, 90, 95, 99, or 100% identity thereto.
      157. The nucleic acid of any of embodiments 145-156, wherein the first region comprises a nucleotide sequence encoding the first targeting moiety, wherein the nucleotide sequence encoding the first targeting moiety comprises a sequence according to SEQ ID NO: 46 or 131 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      158. The nucleic acid of any of embodiments 145-157, wherein the first region comprises a nucleotide sequence encoding the first effector moiety, wherein the nucleotide sequence encoding the first effector moiety comprises a sequence according to SEQ ID NO: 52 or 132, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      159. The nucleic acid of any of embodiments 145-158, wherein the second region comprises a nucleotide sequence encoding the second targeting moiety, wherein the nucleotide sequence encoding the second targeting moiety comprises a sequence according to SEQ ID NO: 40 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      160. The nucleic acid of any of embodiments 145-159, wherein the first region comprises a nucleotide sequence encoding the first effector moiety, wherein the nucleotide sequence encoding the first effector moiety comprises a sequence according to SEQ ID NO: 51, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      161. The nucleic acid of any of embodiments 145-160, wherein the first region comprises a nucleotide sequence according to SEQ ID NO: 63 or 130, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
      162. The nucleic acid of any of embodiments 145-161, wherein the second region comprises a nucleotide sequence according to SEQ ID NO: 57, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
      163. A nucleic acid encoding a system for reducing MYC expression, the nucleic acid comprising:
a) a first region encoding a first expression repressor, the first expression repressor comprising:
   i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second region encoding a second expression repressor, the second expression repressor comprising:
   i) a second targeting moiety having an amino acid sequence according to SEQ ID NO:169 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   ii) a second effector moiety having an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      164. A nucleic acid encoding a system for reducing MYC expression, the nucleic acid comprising:
a) a first region encoding a first expression repressor, the first expression repressor comprising:
   i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second region encoding a second expression repressor, the second expression repressor comprising:
   i) a second targeting moiety having an amino acid sequence according to SEQ ID NO:171 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   ii) a second effector moiety having an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      165. The nucleic acid of embodiment 163 or 164, wherein the first region is 5′ of the second region.
      166. The nucleic acid of embodiment 163 or 164, wherein the first region is 3′ of the second region.
      167. The nucleic acid of any of embodiments 163-166, wherein the first region further comprises a nucleotide sequence encoding a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the first targeting moiety.
      168. The nucleic acid of any of embodiments 163-167, wherein the first region further comprises a nucleotide sequence encoding a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the first effector moiety.
      169. The nucleic acid of any of embodiments 163-168, wherein the second region further comprises a nucleotide sequence encoding a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the second targeting moiety.
      170. The nucleic acid of any of embodiments 163-169, wherein the second region further comprises a nucleotide sequence encoding a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the second effector moiety.
      171. The nucleic acid of any of embodiments 163-170, wherein the first region further comprises a nucleotide sequence encoding a first linker situated between the first targeting moiety and the first effector moiety, wherein optionally the first linker has an amino acid sequence according to SEQ ID NO: 137 or a sequence with at least 80, 85, 90, 95, 99, or 100% identity thereto.
      172. The nucleic acid of any of embodiments 163-171, wherein the second region further comprises a nucleotide sequence encoding a second linker situated between the second targeting moiety and the second effector moiety, wherein optionally the second linker has an amino acid sequence according to SEQ ID NO: 138 or a sequence with at least 80, 85, 90, 95, 99, or 100% identity thereto.
      173. The nucleic acid of any of embodiments 163-171, wherein the first region further comprises a nucleotide sequence encoding an amino acid sequence C-terminal of the first effector moiety, e.g., a sequence of up to 30, 25, 20, or 18 amino acids, e.g., a sequence according to SEQ ID NO: 126 or a sequence with at least 80, 85, 90, 95, 99, or 100% identity thereto.
      174. The nucleic acid of any of embodiments 163-173, wherein the second region further comprises a nucleotide sequence encoding an amino acid sequence N-terminal of the second targeting moiety, e.g., a sequence of up to 30, 25, 20, or 18 amino acids, e.g., a sequence according to SEQ ID NO: 128 or a sequence with at least 80, 85, 90, 95, 99, or 100% identity thereto.
      175. The nucleic acid of any of embodiments 163-174, wherein the first expression repressor has an amino acid sequence according SEQ ID NO: 30 or 129, or a sequence with at least 80, 85, 90, 95, 99, or 100% identity thereto.
      176. The nucleic acid of any of embodiments 144-175, wherein the second expression repressor has an amino acid sequence according to SEQ ID NO: 177, or 183 or a sequence with at least 80, 85, 90, 95, 99, or 100% identity thereto.
      177. The nucleic acid of any of embodiments 144-176, wherein the second expression repressor has an amino acid sequence according to SEQ ID NO: 179, or 185, or a sequence with at least 80, 85, 90, 95, 99, or 100% identity thereto.
      178. The nucleic acid of any of embodiments 144-177, wherein first expression repressor comprises an amino acid sequence according to SEQ ID NO: 30, or 129, or a sequence with at least 80, 85, 90, 95, 99, or 100% identity thereto and the second expression repressor has an amino acid sequence according to SEQ ID NO: 24, 141, 177, 179, 183, or 185, or a sequence with at least 80, 85, 90, 95, 99, or 100% identity thereto.
      179. The nucleic acid of any of embodiments 144-178, wherein the first region comprises a nucleotide sequence encoding the first targeting moiety, wherein the nucleotide sequence encoding the first targeting moiety comprises a sequence according to SEQ ID NO: 46 or 131 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      180. The nucleic acid of any of embodiments 144-179, wherein the first region comprises a nucleotide sequence encoding the first effector moiety, wherein the nucleotide sequence encoding the first effector moiety comprises a sequence according to SEQ ID NO: 52 or 132, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      181. The nucleic acid of any of embodiments 144-180, wherein the second region comprises a nucleotide sequence according to SEQ ID NO: 173 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
      182. The nucleic acid of any of embodiments 144-181, wherein the second region comprises a nucleotide sequence according to SEQ ID NO: 175 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
      183. The nucleic acid of any of embodiments 144-182, wherein the second region comprises a nucleotide sequence encoding the second effector moiety, wherein the nucleotide sequence encoding the second effector moiety comprises a sequence according to SEQ ID NO: 51, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      184. The nucleic acid of any of embodiments 144-183, wherein the first region comprises a nucleotide sequence according to SEQ ID NO: 63 or 130, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
      185. The nucleic acid of any of embodiments 144-184, wherein the second region comprises a nucleotide sequence according to SEQ ID NO: 189, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
      186. The nucleic acid of any of embodiments 144-185, wherein the second region comprises a nucleotide sequence according to SEQ ID NO: 194, or a sequence with at least 80, 85, 90, 95, 99, or 100% identity Thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional 187. The nucleic acid of any of embodiments 144-186, wherein the first region comprises a nucleotide sequence encoding the first effector moiety, wherein the nucleotide sequence encoding the first effector moiety comprises a sequence according to SEQ ID NO: 52 or 132 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      188. The nucleic acid of any of embodiments 144-187, wherein the first region comprises a nucleotide sequence encoding the first targeting moiety, wherein the nucleotide sequence encoding the first targeting moiety comprises a sequence according to SEQ ID NO: 46 or 131, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      189. The nucleic acid of any of embodiments 144-188, wherein the second region comprises a nucleotide sequence encoding the second effector moiety, wherein the nucleotide sequence encoding the second effector moiety comprises a sequence according to SEQ ID NO: 51 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      190. The nucleic acid of any of embodiments 144-189, wherein the second region comprises a nucleotide sequence according to SEQ ID NO: 189, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      191. The nucleic acid of any of embodiments 144-190, wherein the second region comprises a nucleotide sequence according to SEQ ID NO: 194, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      192. The nucleic acid of any of embodiments 144-191, which has a nucleotide sequence according to SEQ ID NO: 93, 112, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      193. The nucleic acid of any of embodiments 144-192, which has a nucleotide sequence according to SEQ ID NO: 196 or 197, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
      194. The system or nucleic acid of any of embodiments 118-193, wherein the first expression repressor comprises the first effector moiety.
      195. The system or nucleic acid of any of embodiments 118-194, wherein the second expression repressor comprises the second effector moiety.
      196. The system or nucleic acid of any of embodiments 118-195, wherein the first effector moiety has a different amino acid sequence from the second effector moiety.
      197. The system or nucleic acid of any of embodiments 118-196, wherein the first effector moiety is a durable effector moiety.
      198. The system or nucleic acid of any of embodiments 118-125 or 144-197, wherein the first effector moiety is a transient effector moiety.
      199. The system or nucleic acid of any of embodiments 118-198, wherein the first effector moiety is an epigenetic modifying moiety.
      200. The system or nucleic acid of any of embodiments 118-143, 163-197 or 199, wherein the first effector moiety comprises a histone methyltransferase.
      201. The system or nucleic acid of embodiment 200, wherein the first effector moiety comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof.
      202. The system or nucleic acid of any of embodiments 118-143, 163-197, or 199, wherein the first effector moiety comprises a histone demethylase (e.g., a lysine demethylase).
      203. The system or nucleic acid of embodiment 202, wherein the first effector moiety comprises a protein chosen from KDM1A (i.e., LSD1), KDM1B (i.e., LSD2), KDM2A, KDM2B, KDM5A, KDM5B, KDM5C, KDM5D, KDM4B, NO66 (or a functional variant or fragment of any thereof).
      204. The system or nucleic acid of any of embodiments 118-143, 163-197, or 199, wherein the first effector moiety comprises a histone deacetylase.
      205. The system or nucleic acid of embodiment 204, wherein the first effector moiety comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.
      206. The system or nucleic acid of any of embodiments 118-197 or 200, wherein the first effector moiety comprises a DNA methyltransferase.
      207. The system or nucleic acid of embodiment 206, wherein the first effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.
      208. The system or nucleic acid of any of embodiments 118-143, 160-196, or 198 or, wherein the first effector moiety is a transcription repressor moiety, e.g., comprising a transcription repressor.
      209. The system or nucleic acid of embodiment 198 or 199, wherein the first effector moiety comprises a protein chosen from KRAB, MeCP2, HP1, RBBP4, REST, FOG1, SUZ12, or a functional variant or fragment of any thereof.
      210. The system or nucleic acid of any of embodiments 118-209, wherein the first effector moiety promotes epigenetic modification of the transcription regulatory element or a sequence proximal thereto.
      211. The system or nucleic acid of any of embodiments 118-210, wherein the first effector moiety catalyzes epigenetic modification of the transcription regulatory element or a sequence proximal thereto.
      212. The system or nucleic acid of any of embodiments 118-125, 194, or 197-211, wherein the second expression repressor does not comprise an effector moiety.
      213. The system or nucleic acid of any of embodiments 118-212, wherein the second effector moiety is a transient effector moiety.
      214. The system or nucleic acid of any of embodiments 118-125 or 194-211, wherein the second effector moiety is a durable effector moiety.
      215. The system or nucleic acid of any of embodiments 118-211 or 214, wherein the second effector moiety is an epigenetic modifying moiety.
      216. The system or nucleic acid of any of embodiments 118-125, 194-211, or 214-215, wherein the second effector moiety comprises a histone methyltransferase.
      217. The system or nucleic acid of embodiment 216, wherein the second effector moiety comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof.
      218. The system or nucleic acid of any of embodiments 118-125, 194-211, or 214-215, wherein the second effector moiety comprises a histone demethylase (e.g., a lysine demethylase).
      219. The system or nucleic acid of embodiment 218, wherein the second effector moiety comprises a protein chosen from KDM1A (i.e., LSD1), KDM1B (i.e., LSD2), KDM2A, KDM2B, KDM5A, KDM5B, KDM5C, KDM5D, KDM4B, NO66 (or a functional variant or fragment of any thereof.
      220. The system or nucleic acid of any of embodiments 118-125, 194-211, or 214-215, wherein the second effector moiety comprises a histone deacetylase.
      221. The system or nucleic acid of embodiment 220, wherein the second effector moiety comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.
      222. The system or nucleic acid of any of embodiments 118-125, 194-211, or 214-215, wherein the second effector moiety comprises a DNA methyltransferase.
      223. The system or nucleic acid of embodiment 222, wherein the second effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.
      224. The system or nucleic acid of any of embodiments 118-211 or 213, wherein the second effector moiety is a transcription repressor moiety.
      225. The system or nucleic acid of embodiment 224, wherein the second effector moiety promotes epigenetic modification of the anchor sequence or a sequence proximal thereto.
      226. The system or nucleic acid of embodiment 223 or 224, wherein the second effector moiety binds to one or more endogenous epigenetic modifying proteins or one or more endogenous transcription modifying proteins.
      227. The system or nucleic acid of any of embodiments 223-226, wherein the second effector moiety comprises KRAB, MeCP2, HP1, RBBP4, REST, FOG1, SUZ12, or a functional variant or fragment of any thereof.
      228. The system or nucleic acid of any of embodiments 118-197, 199-207, 210-211, 213, or 224-227, wherein:
the first effector moiety is a durable effector moiety, and
the second effector moiety is a transient effector moiety.
   229. The system or nucleic acid of embodiment 228, wherein the first effector moiety is an epigenetic modifying moiety.
   230. The system or nucleic acid of embodiment 227 or 228, wherein the second effector moiety is a transcription repressor moiety.
   231. The system or nucleic acid of any of embodiments 227-230, wherein:
the first effector moiety comprises a histone methyltransferase, histone demethylase, histone deacetylase, DNA methyltransferase, a functional variant or fragment of any thereof, or a combination of any thereof, and
the second effector moiety comprises a transcription repressor or a functional variant or fragment of any thereof.
   232. The system or nucleic acid of any of embodiments 118-125, 194, 197, 199-207, 210-212, or 190, wherein:
the first effector moiety comprises a histone methyltransferase, histone demethylase, histone deacetylase, DNA methyltransferase, a functional variant or fragment of any thereof, or a combination of any thereof, and
the second expression repressor does not comprise a second effector moiety.
   233. The system or nucleic acid of any of embodiments 118-125, 199-207, 210-211, 213 214, or 224-231 wherein:
the first effector moiety comprises a SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, NO66, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, a functional variant or fragment of any thereof, or a combination of any thereof, and the second effector moiety comprises KRAB, MeCP2, HP1, RBBP4, REST, FOG1, SUZ12, a functional variant or fragment of any thereof, or a combination of any thereof.
   234. The system or nucleic acid of any of embodiments 118-197, 199, 206-207, 210-211, 213, 215, 224-231, or 233, wherein:
the first effector moiety comprises a DNA methyltransferase, and
the second effector moiety comprises a transcription repressor.
   235. The system or nucleic acid of any of embodiments 118-125, 194, 197, 200, 206-207, 210-212, or 232 wherein:
the first effector moiety comprises a DNA methyltransferase, and
the second expression repressor does not comprise a second effector moiety.
   236. The system or nucleic acid of any of embodiments 118-125, 200, 206-207, 210-235 wherein the first effector moiety comprises MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.
   237. The system or nucleic acid of any of embodiments 118-211, 214, 224-234, or 236, wherein the second effector moiety comprises KRAB, MeCP2, HP1, RBBP4, REST, FOG1, SUZ12, or a functional variant or fragment of any thereof.
   238. The system or nucleic acid of any of embodiments 118-211, 199, 206-207, 210-211, 213, 224-234, or 236-237, wherein:
the first effector moiety comprises MQ1 or a functional variant or fragment of any thereof, and
the second effector comprises KRAB or a functional variant or fragment of any thereof.
   239. The system or nucleic acid of any of embodiments 118-125, 194, 197, 199-207, or 210-212, 229, 232, 235, or 236, wherein:
the first effector moiety comprises MQ1 or a functional variant or fragment of any thereof, and
the second expression repressor does not comprise a second effector moiety.
   240. The system or nucleic acid of any of embodiments 118-200 wherein the first expression repressor comprises an amino acid sequence chosen from any of SEQ ID NOs: 22-37, 129, 133, 134, 139-149, 177-180, or 183-186, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   241. The system or nucleic acid of any of embodiments 118-198, 200, 206-211, 213-216, 222-223, 236-237, or 240, wherein the second expression repressor comprises an amino acid sequence chosen from any of SEQ ID NOs: 22-37, 129, 133, 134, 139-149, 177-180, or 183-186, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   242. The system or nucleic acid of any of embodiments 118-198, 200, 206-211, 213-216, 222-223, 236-237, or 240-241, wherein the first expression repressor comprises an amino acid sequence of SEQ ID NO: 30, 129, 133, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second expression repressor comprises an amino acid sequence of SEQ ID NO: 24, 134, 141, 177, 179, 183, or 185, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   243. The system or nucleic acid of any of embodiments 118-198, 200, 206-211, 213-216, 222-223, 236-237, or 240-242, wherein the first expression repressor is encoded by a first nucleotide sequence of SEQ ID NO: 63 or 130, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second expression repressor are encoded by a second nucleotide sequence of SEQ ID NO: 57, 189, or 194, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   244. The system or nucleic acid of any of embodiments 118-198, 200, 206-211, 213-216, 222-223, 236-237, or 240-243, wherein the first and the second repressor are encoded by a nucleic acid sequence of SEQ ID NO: 93, 94, 112, 113, 196, or 197, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   245. The system or nucleic acid of embodiment 244 comprising an amino acid sequence of SEQ ID NO: 91, 92, 121, 122, 181, 182, 187, or 188, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   246. The system or nucleic acid of any of embodiments 118-197, 199, 206-207, 210-211, 213, 215, 224-231, 233-234, 236-237, or 240-244, wherein:
the first expression repressor comprises from N-terminus to C-terminus:
(i) a first nuclear localization signal, e.g., a SV40 NLS; e.g., a sequence according to SEQ ID NO: 135;
(ii) a first targeting moiety, e.g., a zinc finger binding domain, e.g., ZF9; e.g., a sequence according to SEQ ID NO: 13;
(iii) a first effector moiety, e.g., a DNA methyltransferase, e.g., MQ1; e.g., a sequence according to SEQ ID NO: 19 or 87;
(iv) a second nuclear localization signal, e.g., a nucleoplasmin NLS; e.g., a sequence according to SEQ ID NO: 136;
and the second expression repressor comprises, from N-terminus to C-terminus:
(v) a third nuclear localization signal, e.g., a SV40NLS; e.g., a sequence according to SEQ ID NO: 135;
(vi) a second targeting moiety, e.g., a zinc finger binding domain, e.g., ZF3; e.g., a sequence according to SEQ ID NO: 7;
(vii) a second effector moiety, e.g., KRAB, e.g., a sequence according to SEQ ID NO:18; and
(viii) a fourth nuclear localization signal, e.g., a nucleoplasmin NLS, e.g., a sequence according to SEQ ID NO: 136.
   247. The system or nucleic acid of any of embodiments 118-197, 199, 206-207, 210-211, 213, 215, 224-231, 233-234, 236-237, or 240-244, wherein:
the first expression repressor comprises from N-terminus to C-terminus:
(i) a first nuclear localization signal, e.g., a SV40 NLS; e.g., a sequence according to SEQ ID NO: 135;
(ii) a first targeting moiety, e.g., a zinc finger binding domain, e.g., ZF9; e.g., a sequence according to SEQ ID NO: 13;
(iii) a first effector moiety, e.g., a DNA methyltransferase, e.g., MQ1; e.g., a sequence according to SEQ ID NO: 19 or 87;
(iv) a second nuclear localization signal, e.g., a nucleoplasmin NLS; e.g., a sequence according to SEQ ID NO: 136;
and the second expression repressor comprises, from N-terminus to C-terminus:
(v) a third nuclear localization signal, e.g., a SV40NLS; e.g., a sequence according to SEQ ID NO: 135;
(vi) a second targeting moiety, e.g., a zinc finger binding domain, e.g., ZF54; e.g., a sequence according to SEQ ID NO: 169;
(vii) a second effector moiety, e.g., KRAB, e.g., a sequence according to SEQ ID NO:18; and
(viii) a fourth nuclear localization signal, e.g., a nucleoplasmin NLS, e.g., a sequence according to SEQ ID NO: 136.
   248. The system or nucleic acid of any of embodiments 118-197, 199, 206-207, 210-211, 213, 215, 224-231, 233-234, 236-237, or 240-244, wherein:
the first expression repressor comprises from N-terminus to C-terminus:
(i) a first nuclear localization signal, e.g., a SV40 NLS; e.g., a sequence according to SEQ ID NO: 135;
(ii) a first targeting moiety, e.g., a zinc finger binding domain, e.g., ZF9; e.g., a sequence according to SEQ ID NO: 13;
(iii) a first effector moiety, e.g., a DNA methyltransferase, e.g., MQ1; e.g., a sequence according to SEQ ID NO: 19 or 87;
(iv) a second nuclear localization signal, e.g., a nucleoplasmin NLS; e.g., a sequence according to SEQ ID NO: 136;
and the second expression repressor comprises, from N-terminus to C-terminus:
(v) a third nuclear localization signal, e.g., a SV40NLS; e.g., a sequence according to SEQ ID NO: 135;
(vi) a second targeting moiety, e.g., a zinc finger binding domain, e.g., ZF67; e.g., a sequence according to SEQ ID NO: 171;
(vii) a second effector moiety, e.g., KRAB, e.g., a sequence according, to SEQ ID NO:18; and
(viii) a fourth nuclear localization signal, e.g., a nucleoplasmin NLS, e.g., a sequence according to SEQ ID NO: 136.
   249. The system of any of embodiment 118-248, wherein the system is capable of the decreasing expression of MYC to a greater degree compared to the first expression repressor alone or the second expression repressor alone.
   250. The system of any of embodiments 128-194, or 242-249, wherein the system is capable of decreasing expression of MYC to a greater degree compared to any of the expression repressors of SEQ ID: 22, 23, 25-29, 31-37 alone or in combination.
   251. The system of any of embodiments 118-250, which is capable of reducing tumor volume, e.g., in a human subject or in a mammalian model.
   252. The system of any of embodiments 128-193 or 242-209, wherein the system is capable of reducing tumor volume to a similar or greater degree compared to a chemotherapeutic agent, e.g., in a mammalian model, e.g., when measured at day 20 after initiation of treatment, e.g., wherein the expression repressor is administered every 5 days at a dose of 3 mg/kg, e.g., in a model system as described in Example 15.
   253. The system of any of embodiments 128-193 or 242-252, wherein the system is capable of reducing tumor volume to a greater degree compared to a chemotherapeutic agent, e.g., in a mammalian model, e.g., when measured at day 15 after initiation of treatment, e.g., wherein the expression repressor is administered every 5 days at a dose of 6 mg/kg, e.g., in a model system as described in Example 14.
   254. The system of any of embodiments 128-193 or 242-253, wherein the tumor volume is reduced by at least about 10%, 20%, 30%, 40%, 50%, or 60% compared to a control treated with PBS, e.g., at day 20 after start of treatment.
   255. The system of embodiment 254 wherein the chemotherapeutic agent is sorafenib or cisplatin.
   256. The system of any of embodiments 128-193 or 242-253, wherein the system is capable of reducing tumor volume to a similar or greater degree compared to a small molecule MYC inhibitor.
   257. The system of embodiment 256 wherein the small molecule MYC inhibitor is MYCi975 wherein optionally tumor volume is reduced by at least about 10%, 20%, 30%, or 40% compared to a control treated with the MYCi975, e.g., at day 20 after start of treatment.
   258. The system of any of embodiments 118-257, which does not cause a decrease in body weight compared to at the start of treatment, or which causes a decrease in body weight of less than 3%, 2%, or 1%.
   259. The system or nucleic acid of any of embodiments 118-258, wherein the first targeting moiety is selected from a TAL effector domain, a CRISPR/Cas domain, a zinc finger domain, a tetR domain, a meganuclease, or an oligonucleotide.
   260. The system or nucleic acid of any of embodiments 118-260, wherein the second targeting moiety is selected from a TAL effector domain, a CRISPR/Cas domain, a zinc finger domain, a tetR domain, a meganuclease, or an oligonucleotide.
   261. The system or nucleic acid of any of embodiments 118-260, wherein the first targeting moiety comprises a CRISPR/Cas domain (e.g., a first CRISPR/Cas domain).
   262. The system or nucleic acid of any of embodiments 118-261, wherein the second targeting moiety comprises a second CRISPR/Cas domain (e.g., a second CRISPR/Cas domain).
   263. The system or nucleic acid of embodiment 262, wherein: i) the first CRISPR/Cas domain binds a first guide RNA, ii) the second CRISPR/Cas domain binds a second guide RNA, or iii) both (i) and (ii).
   264. The system or nucleic acid of embodiment 262 or 263, wherein the first CRISPR/Cas domain dues not bind the second guide RNA or binds with a K_{D} of at least 10, 20, 50, 100, 1000, or 10,000 nM, and the second CRISPR/Cas domain does not bind the first guide RNA, or binds with a K_{D} of at least 10, 20, 50, 100, 1000, or 10,000 nM.
   265. The system or nucleic acid of any of embodiments 260-264, wherein the first CRISPR/Cas domain comprises a different amino acid sequence than the second CRISPR/Cas domain.
   266. The system or nucleic acid of any of embodiments 260-265, wherein the first or second CRISPR/Cas domain comprises an amino acid sequence of a Cas protein or Cpf1 protein chosen from Table 1 or a variant (e.g., mutant) of any thereof.
   267. The system or nucleic acid of any of embodiments 260-266, wherein the first CRISPR/Cas domain comprises an amino acid sequence of a Cas protein or Cpf1 protein chosen from Table 1 or a variant (e.g., mutant) of any thereof, and the second CRISPR/Cas domain comprises an amino acid sequence of a different Cas protein or Cpf1 protein chosen from Table 1 or a variant (e.g., mutant) of any thereof.
   268. The system or nucleic acid of any of embodiments 118-260, wherein the first targeting moiety comprises a zinc finger domain (e.g., a first zinc finger domain).
   269. The system or nucleic acid of any of embodiments 118-260 or 268, wherein the second targeting moiety comprises a zinc finger domain (e.g., a second zinc finger domain).
   270. The system or nucleic acid of any of embodiments 118-261 or 268-269, wherein the first targeting moiety comprises a first zinc finger domain and the second targeting moiety comprises a second zinc finger domain.
   271. The system or nucleic acid of any of embodiments 268-270, wherein the first zinc finger domain and the second zinc finger domain bind the same genomic locus, e.g., have the same amino acid sequence.
   272. The system or nucleic acid of any of embodiments 268-271, wherein the first zinc finger domain and the second zinc finger domain have different amino acid sequences or bind different genomic loci.
   273. The system or nucleic acid of any of embodiments 118-261 or 267-272, wherein the first zinc finger molecule comprises at least 1, 2, 3, 4, 5, 7, 8, 9, or 10 zinc fingers (and optionally no more than 11, 10, 9, 8, 7, 6, or 5 zinc fingers).
   274. The system or nucleic acid of any of embodiments 267-273, wherein the first zinc finger molecule comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7- 9, 7-8, 8-10, 8-9, or 9-10 zinc fingers.
   275. The system or nucleic acid of any of embodiments 268-274, wherein the first zinc finger domain comprises 3 or 9 zinc fingers.
   276. The system or nucleic acid of any of embodiments 268-275, wherein the second zinc finger domain comprises at least 1, 2, 3, 4, 5, 7, 8, 9, or 10 zinc fingers (and optionally no more than 11, 10, 9, 8, 7, 6, or 5 zinc fingers).
   277. The system or nucleic acid of any of embodiments 268-276, wherein the second zinc finger domain comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7- 9, 7-8, 8-10, 8-9, or 9-10 zinc fingers.
   278. The system or nucleic acid of any of embodiments 268-277, wherein the second zinc finger domain comprises 3 or 9 zinc fingers.
   279. The system or nucleic acid of any of embodiments 118-278, wherein the first targeting moiety comprises a TAL effector domain (e.g., a first TAL effector domain).
   280. The system or nucleic acid of any of embodiments 118-260 or 279 wherein the second targeting moiety comprises a TAL effector domain (e.g., a second TAL effector domain).
   281. The system or nucleic acid of any of embodiments 279 or 280, wherein the first TAL effector domain comprises at least 2, 4, 6, 8, 10, 12, 14, 16, 18, 20, 22, 24, 26, 28, 30, 32, 34, 36, 38, or 40 central repeats (and optionally, no more than 45, 40, 35, 30, 25, 20, 15, or 10 central repeats).
   282. The system or nucleic acid of any of embodiments 279-281, wherein the first TAL effector domain comprises 2-40, 5-40, 10-40, 15-40, 20-40, 25-40, 30-40, 35-40, 2-35, 5-35, 10-35, 15-35, 20-35, 25-35, 30-35, 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 central repeats.
   283. The system or nucleic acid of any of embodiments 279-282, wherein the second TAL effector domain comprises at least 2, 4, 6, 8, 10, 12, 14, 16, 18, 20, 22, 24, 26, 28, 30, 32, 34, 36, 38, or 40 central repeats (and optionally, no more than 45, 40, 35, 30, 25, 20, 15, or 10 central repeats).
   284. The system or nucleic acid of any of embodiments 279-283, wherein the second TAL effector domain comprises 2-40, 5-40, 10-40, 15-40, 20-40, 25-40, 30-40, 35-40, 2-35, 5-35, 10-35, 15-35, 20-35, 25-35, 30-35, 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 central repeats.
   285. The system or nucleic acid of any of embodiments 118-284, wherein the first targeting moiety comprises a nucleic acid (e.g., a first nucleic acid).
   286. The system of any of embodiments 129-285, wherein the second targeting moiety comprises a nucleic acid (e.g., a second nucleic acid).
   287. The system or nucleic acid of any of embodiments 129-286, wherein the first targeting moiety comprises a polypeptide (e.g., a first polypeptide).
   288. The system or nucleic acid of any of embodiments 129-287, wherein the second targeting moiety comprises a polypeptide (e.g., a second polypeptide).
   289. The system of embodiment 287 or 288, wherein the nucleic acid is covalently attached to the polypeptide.
   290. The system of embodiment 288 or 289, wherein the nucleic acid is non-covalently associated with the polypeptide.
   291. The system or nucleic acid of any of embodiments 275-290, wherein the nucleic acid comprises a sequence that is complementary to the transcriptional regulatory element or a sequence proximal thereto, or comprises no more than 10, 9, 8, 7, 6, 5, 4, 3, 2, or 1 mismatches relative to the transcriptional regulatory element or a sequence proximal thereto.
   292. The system or nucleic acid of any of embodiments 275-291, wherein the nucleic acid comprises a sequence that is complementary to the anchor sequence or a sequence proximal thereto, or comprises no more than 10, 9, 8, 7, 6, 5, 4, 3, 2, or 1 mismatches relative to the anchor sequence or a sequence proximal thereto.
   293. The system of any of embodiments 275-292, wherein the nucleic acid comprises DNA, a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA, or other RNAi molecule.
   294. The system of any of embodiments—275-293, wherein the nucleic acid comprises a gRNA.
   295. The system of any of embodiments 275-294, wherein the nucleic acid comprises a sequence with at least 80, 85, 90, 95, 99, or 100% identity to any of SEQ ID NOs: 1-4, or has no more than 1, 2, 3, 4, 5, 6, 7, 8, 9, or 10 positions of difference thereto.
   296. The system of any of embodiments 275-295, wherein the first nucleic acid comprises a sequence with at least 80, 85, 90, 95, 99, or 100% identity to any of SEQ ID NOs: 1-4 or has no more than 1, 2, 3, 4, 5, 6, 7, 8, 9, or 10 positions of difference thereto, and the second nucleic acid comprises a sequence with at least 80, 85, 90, 95, 99, or 100% identity to any of SEQ ID NOs: 1-4 or has no more than 1, 2, 3, 4, 5, 6, 7, 8, 9, or 10 positions of difference thereto.
   297. The system of any of embodiments 275-295, wherein the first nucleic acid comprises a sequence with at least 80, 85, 90, 95, 99, or 100% identity to any of SEQ ID NOs: 96-110 or has no more than 1, 2, 3, 4, 5, 6, 7, 8, 9, or 10 positions of difference thereto, and the second nucleic acid comprises a sequence with at least 80, 85, 90, 95, 99, or 100% identity to any of SEQ ID NOs: 96-110 or has no more than 1, 2, 3, 4, 5, 6, 7, 8, 9, or 10 positions of difference thereto.
   298. The system of any of embodiments 118-297, wherein the transcriptional regulatory element comprises a promoter.
   299. The system of any of embodiments 118-298, wherein the transcriptional regulatory element comprises an enhancer; e.g., a super enhancer.
   300. The system of any of embodiments 118-299, wherein the anchor sequence comprises a CTCF binding motif.
   301. The system of any of embodiments 118-300, wherein the anchor sequence comprises a YY1 binding motif.
   302. The system of any of embodiments 118-301, wherein the anchor sequence comprises the sequence of SEQ ID NO: 71 or 72, or a sequence with no more than 8, 7, 6, 5, 4, 3, 2, or 1 alterations relative thereto.
   303. The system of any of embodiments 118-302, wherein the anchor sequence comprises a sequence according to SEQ ID NO: 73 or 74, or a sequence with no more than 8, 7, 6, 5, 4, 3, 2, or 1 alterations relative thereto.
   304. The system of any of embodiments 118-303, wherein the anchor sequence is on the same chromosome as the MYC gene.
   305. The system of any of embodiments 118-304, wherein the anchor sequence is upstream of the MYC gene (e.g., upstream of the TSS or upstream of the promoter).
   306. The system of any of embodiments 118-305, wherein the anchor sequence is at least 1, 5, 10, 50, 100, or 1000 kilobases away from the MYC gene (e.g., from the TSS or promoter of the MYC gene).
   307. The system of any of embodiments 118-306, wherein the anchor sequence is 0.1-0.5, 0.1-1, 0.1-5, 0.1-10, 0.1-50, 0.1-100, 0.1-500, 0.1-1000, 0.5-1, 0.5-5, 0.5-10, 0.5-50, 0.5-100, 0.5-500, 0.5-1000, 1-5, 1-10, 1-50, 1-100, 1-500, 1-1000, 5-10, 5-50, 5-100, 5-500, 5-1000, 10-50, 10-100, 10-500, 10-1000, 50-100, 50-500, 50-1000, 100-500, 100-1000, or 500-1000 kilobases away from the MYC gene (e.g., from the TSS or promoter of the MYC gene).
   308. The system of any of embodiments 118-303 or 305-307, wherein the anchor sequence is on a different chromosome than the MYC gene.
   309. The system of any of embodiments 118-308, wherein the second targeting moiety binds to the anchor sequence or a sequence proximal to the anchor sequence with affinity sufficient to compete for binding with an endogenous polypeptide (e.g., CTCF or YY1).
   310. The system of any of embodiments 118-309, wherein the first targeting moiety binds to a sequence at chromosome coordinates 128746342-128746364, 128746321-128746343, or 128746525-128746547, or a sequence proximal thereto.
   311. The system of any of embodiments 118-309, wherein the first targeting moiety binds to a sequence at chromosome coordinates 128746405-128746425, 128748069-128748089, 129188825-129188845, or 129188822-129188842 or a sequence proximal thereto.
   312. The system of any of embodiments 118-311, wherein the second targeting moiety binds to a sequence at chromosome coordinates 128748014-128748036, or a sequence proximal thereto.
   313. The system of any of embodiments 118-311, wherein the second targeting moiety binds to a sequence at chromosome coordinates 128746405-128746425, 128748069-128748089, 129188825-129188845, or 129188822-129188842, or a sequence proximal thereto.
   314. The system of any of embodiments 118-314, wherein the first expression repressor is a fusion molecule.
   315. The system of any of embodiments 118-314, wherein the second expression repressor is a fusion molecule.
   316. The system of any of embodiments 118-315, wherein the first expression repressor comprises a linker.
   317. The system of any of embodiments 118-316, wherein the second expression repressor comprises a linker.
   318. The system of any of embodiments 118-267 or 285-317, wherein:
the first expression repressor comprises a targeting moiety comprising a first CRISPR/Cas molecule, e.g., comprising a first catalytically inactive CRISPR/Cas protein, and an effector moiety comprising an epigenetic modifying moiety; and
the second expression repressor comprises a targeting moiety comprising a second CRISPR/Cas molecule, e.g., comprising a second catalytically inactive CRISPR/Cas protein, and an optionally an effector moiety comprising a transcription repressor.
   319. The system of any of embodiments 118-260, 268-278, or 285-317 wherein:
the first expression repressor comprises a targeting moiety comprising a first zinc finger domain, and an effector moiety comprising an epigenetic modifying moiety; and
the second expression repressor comprises a targeting moiety comprising a second zinc finger domain, and optionally an effector moiety comprising a transcription repressor.
   320. The system of any of embodiments 118-120, 262, 268, or 275-318, wherein:
the first expression repressor comprises a targeting moiety comprising a CRISPR/Cas molecule, e.g., comprising a catalytically inactive CRISPR/Cas protein, and an effector moiety comprising an epigenetic modifying moiety; and
the second expression repressor comprises a targeting moiety comprising a zinc finger domain, and optionally an effector moiety comprising a transcription repressor.
   321. The system of any of embodiments 118-260, 268, or 275-318, wherein:
the first expression repressor comprises a targeting moiety comprising a zinc finger domain, and an effector moiety comprising an epigenetic modifying moiety; and
the second expression repressor comprises a targeting moiety comprising a CRISPR/Cas domain, e.g., comprising a catalytically inactive CRISPR/Cas protein, and optionally an effector moiety comprising a transcription repressor.
   322. The system of any of embodiments 260, 268-278, or 275-318, wherein the zinc finger domain (e.g., the first or second zinc finger domain) comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 zinc fingers, e.g., 3 or 9 zinc fingers.
   323. The system of any of embodiments 322, wherein the epigenetic modifying moiety comprises a DNA methyltransferase.
   324. The system of any of embodiments 118-323, wherein the epigenetic modifying moiety comprises MQ1 or a functional variant or fragment thereof.
   325. The system of any of embodiments 118-324, wherein the second expression repressor comprises an effector moiety comprising a transcription repressor.
   326. The system of any of embodiments 118-323, wherein the transcription repressor comprises KRAB or a functional variant or fragment thereof.
   327. The system of any of embodiments 118-326, wherein the first expression repressor comprises an amino acid sequence of any of SEQ ID NOS: 28-33 or 35-37, 145-149, 151, 152, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
   328. The system of any of embodiments 118-327, wherein the second expression repressor comprises an amino acid sequence of any of SEQ ID NOS: 22-27, 34, 139-144, 150, 177-180, 183-186, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
   329. The system of any of embodiments 118-328, wherein binding of the first expression repressor to the transcription regulatory element or a sequence proximal thereto decreases expression of MYC in a cell.
   330. The system of embodiment 327, wherein expression is decreased by 10, 20, 30, 40, 50, 60, 70, 80, 90, or 100% compared to expression in the absence of the first expression repressor, e.g., as measured by QPCR or ELISA.
   331. The system of embodiment 326 or 327, wherein binding of the first expression repressor to the transcription regulatory element appreciably decreases expression of MYC for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by QPCR or ELISA.
   332. The system of any of embodiments 329-331, wherein binding of the first expression repressor to the transcription regulatory element appreciably decreases expression of MYC at 1, 2, 3, 4, 5, 6, 7, 8, 10, 12, 16, 20, 24, 28, 32, 36, 40, 44, 48, 52, 56, 60, 64, 68, 72, 76, 80, or 96 hours post-transfection.
   333. The system of any of embodiments 328-332, wherein binding of the second expression repressor to the anchor sequence or a sequence proximal thereto decreases expression of MYC in a cell.
   334. The system of embodiment 333, wherein expression is decreased by 10, 20, 30, 40, 50, 60, 70, 80, 90, or 100% compared to expression in the absence of the second expression repressor, e.g., as measured by QPCR or ELISA.
   335. The system of embodiment 333 or 334, wherein binding of the second expression repressor to the anchor sequence or a sequence proximal thereto appreciably decreases expression of MYC for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by QPCR or ELISA.
   336. The system of any of embodiments 334-335, wherein binding of the second expression repressor to the anchor sequence or a sequence proximal thereto appreciably decreases expression of MYC at 1, 2, 3, 4, 5, 6, 7, 8, 10, 12, 16, 20, 24, 28, 32, 36, 40, 44, 48, 52, 56, 60, 64, 68, 72, 76, 80, or 96 hours post-transfection.
   337. The system of any of embodiments 329-336, wherein binding of the first expression repressor to the transcription regulatory element or a sequence proximal thereto and the second expression repressor to the anchor sequence or a sequence proximal thereto decreases expression of MYC in a cell.
   338. The system of any of embodiments 329-337, wherein binding of the first expression repressor to the transcription regulatory element or a sequence proximal thereto and binding of the second expression repressor to the anchor sequence or a sequence proximal thereto appreciably decreases expression of MYC at 1, 2, 3, 4, 5, 6, 7, 8, 10, 12, 16, 20, 24, 28, 32, 36, 40, 44, 48, 52, 56, 60, 64, 68, 72, 76, 80, or 96 hours post-transfection.
   339. The system of any of embodiments 337 or 338, wherein expression is decreased by 10, 20, 30, 40, 50, 60, 70, 80, 90, or 100% compared to expression in the absence of the first and second expression repressors, e.g., as measured by QPCR or ELISA.
   340. The system of any of embodiments 329-339, wherein binding of the first expression repressor to the transcription regulatory element or a sequence proximal thereto and the second expression repressor to the anchor sequence or a sequence proximal thereto appreciably decreases expression of MYC for a time period of at least 1, 2, 3, 4, 5, 6, 7, 8, 9, 10, 11, or 12 hours, or at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by QPCR or ELISA.
   341. The system of any of embodiments 329-340, wherein the decrease in expression resulting from the binding of the first expression repressor to the transcription regulatory element or a sequence proximal thereto and the second expression repressor to the anchor sequence or a sequence proximal thereto is greater than the decrease in expression resulting from the binding of the first expression repressor to the transcription regulatory element or a sequence proximal thereto or the binding of the second expression repressor to the anchor sequence or a sequence proximal thereto individually.
   342. The system of embodiment 341, wherein the binding of the first expression repressor to the transcription regulatory element or a sequence proximal thereto and the second expression repressor to the anchor sequence or a sequence proximal thereto decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the transcription regulatory element or a sequence proximal thereto or the binding of the second expression repressor to the anchor sequence or a sequence proximal thereto individually, e.g., as measured by QPCR or ELISA.
   343. The system of any of embodiments 329-342, wherein the decrease in expression resulting from the binding of the first expression repressor to the transcription regulatory element or a sequence proximal thereto and the second expression repressor to the anchor sequence or a sequence proximal thereto persists for a longer time (e.g., more hours, days, or cell divisions) than the decrease in expression resulting from the binding of the first expression repressor to the transcription regulatory element or a sequence proximal thereto or the binding of the second expression repressor to the anchor sequence or a sequence proximal thereto individually.
   344. The system of embodiment 343, wherein the binding of the first expression repressor to the transcription regulatory element or a sequence proximal thereto and the second expression repressor to the anchor sequence or a sequence proximal thereto decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× longer (e.g., as measured in hours, days, or cell divisions) than either the binding of the first expression repressor to the transcription regulatory element or a sequence proximal thereto or the binding of the second expression repressor to the anchor sequence or a sequence proximal thereto individually, e.g., as measured by QPCR or ELISA.
   345. The system of any of embodiments 329-344, wherein binding of the first expression repressor to the promoter or a sequence proximal thereto and the second expression repressor to the super-enhancer or a sequence proximal thereto appreciably decreases expression of MYC for a time period of at least 1, 2, 3, 4, 5, 6, 7, 8, 9, 10, 11, or 12 hours, or at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by QPCR or ELISA.
   346. The system of any of embodiments 329-345, wherein the decrease in expression resulting from the binding of the first expression repressor to the promoter or a sequence proximal thereto and the second expression repressor to the super-enhancer or a sequence proximal thereto is greater than the decrease in expression resulting from the binding of the first expression repressor to the promoter or a sequence proximal thereto or the binding of the second expression repressor to the super-enhancer or a sequence proximal thereto individually.
   347. The system of embodiment 346, wherein the binding of the first expression repressor to the promoter or a sequence proximal thereto and the second expression repressor to the super-enhancer or a sequence proximal thereto decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the promoter or a sequence proximal thereto or the binding of the second expression repressor to the super-enhancer or a sequence proximal thereto individually, e.g., as measured by QPCR or ELISA.
   348. The system of any of embodiments 329-347, wherein the decrease in expression resulting from the binding of the first expression repressor to the promoter or a sequence proximal thereto and the second expression repressor to the super-enhancer or a sequence proximal thereto persists for a longer time (e.g., more hours, days, or cell divisions) than the decrease in expression resulting from the binding of the first expression repressor to the promoter or a sequence proximal thereto or the binding of the second expression repressor to the super-enhancer or a sequence proximal thereto individually.
   349. The system of embodiment 348, wherein the binding of the first expression repressor to the promoter or a sequence proximal thereto and the second expression repressor to the super-enhancer or a sequence proximal thereto decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× longer (e.g., as measured in hours, days, or cell divisions) than either the binding of the first expression repressor to the promoter or a sequence proximal thereto or the binding of the second expression repressor to the super-enhancer or a sequence proximal thereto individually, e.g., as measured by QPCR or ELISA.
   350. The system of any of embodiments 329-349, wherein expression is appreciably decreased indefinitely (e.g., for a time period greater than can be experimentally measured).
   351. The system of any of embodiments 329-350, wherein binding of the first expression repressor to the transcription regulatory element or a sequence proximal thereto decreases the viability of a cell comprising the transcription regulatory element or a sequence proximal thereto.
   352. The system of any of embodiments 329-351, wherein contacting a plurality of cells with the first expression repressor or a nucleic acid encoding the first expression repressor decreases the viability of the plurality of cells, optionally wherein the plurality of cells comprise cancerous and non-cancerous cells and/or infected cells and uninfected cells.
   353. The system of embodiment 352, wherein viability is decreased by 10, 20, 30, 40, 50, 60, 70, 80, 90, or 100% compared to viability in the absence of the first expression repressor, e.g., as measured by CellTiter Glo.
   354. The system of any of embodiments 329-353, wherein, administration of the first expression repressor results in apoptosis of at least 5%, 6%, 7%, 8%, 9% 10%, 12%, 15%, 17% 20%, 25% 30%, 40%, 45%, 50%, 55%, 60%, 65%, 75% of target cells (e.g., cancer cells).
   355. The system of any of embodiments 329-354, wherein binding of the second expression repressor to the anchor sequence or a sequence proximal thereto decreases the viability of a cell comprising the anchor sequence or a sequence proximal thereto.
   356. The system of any of embodiments 329-355, wherein contacting a plurality of cells with the second expression repressor or a nucleic acid encoding the second expression repressor decreases the viability of the plurality of cells.
   357. The system of any of embodiments 329-356, wherein binding of the second expression repressor to the super-enhancer or a sequence proximal thereto decreases the viability of a cell comprising the transcription regulatory element or a sequence proximal thereto.
   358. The system of any of embodiments 329-357, wherein contacting a plurality of cells with the second expression repressor or a nucleic acid encoding the first expression repressor decreases the viability of the plurality of cells, optionally wherein the plurality of cells comprise cancerous and non-cancerous cells and/or infected cells and uninfected cells.
   359. The system of embodiment 358, wherein viability is decreased by 10, 20, 30, 40, 50, 60, 70, 80, 90, or 100% compared to viability in the absence of the second expression repressor, e.g., as measured by CellTiter Glo.
   360. The system of any of embodiments 329-359, wherein, administration of the second expression repressor results in apoptosis of at least 5%, 6%, 7%, 8%, 9% 10%, 12%, 15%, 17% 20%, 25% 30%, 40%, 45%, 50%, 55%, 60%, 65%, 75% of target cells (e.g., cancer cells).
   361. The system of any of embodiments 329-360, wherein binding of the first expression repressor to the transcription regulatory element or a sequence proximal thereto and the second expression repressor to the anchor sequence or a sequence proximal thereto decreases the viability of a cell comprising the anchor sequence or a sequence proximal thereto.
   362. The system of any of embodiments 329-361, wherein binding of the first expression repressor to the promoter or a sequence proximal thereto and the second expression repressor to the super-enhancer or a sequence proximal thereto decreases the viability of a cell
   363. The system of any of embodiments 329-362, wherein contacting a plurality of cells with the system or a nucleic acid encoding the system decreases the viability of the plurality of cells.
   364. The system of embodiments 329-363, wherein viability is decreased by 10, 20, 30, 40, 50, 60, 70, 80, 90, or 100% compared to viability in the absence of the system, e.g., as measured by CellTiter Glo.
   365. The system of any of embodiments 329-364, wherein the decrease in viability resulting from the binding of the first expression repressor to the transcription regulatory element or a sequence proximal thereto and the second expression repressor to the anchor sequence or a sequence proximal thereto is greater than the decrease in viability resulting from the binding of the first expression repressor to the transcription regulatory element or a sequence proximal thereto or the binding of the second expression repressor to the anchor sequence or a sequence proximal thereto individually.
   366. The system of any of embodiments 329-365, wherein the decrease in viability resulting from the binding of the first expression repressor to the promoter or a sequence proximal thereto and the second expression repressor to the super-enhancer or a sequence proximal thereto is greater than the decrease in viability resulting from the binding of the first expression repressor to the promoter or a sequence proximal thereto or the binding of the second expression repressor to the super-enhancer or a sequence proximal thereto individually.
   367. The system of embodiment 366, wherein the binding of the first expression repressor to the transcription regulatory element or a sequence proximal thereto and the second expression repressor to the anchor sequence or a sequence proximal thereto decreases viability 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the transcription regulatory element or a sequence proximal thereto or the binding of the second expression repressor to the anchor sequence or a sequence proximal thereto individually, e.g., as measured by CellTiter Glo.
   368. The system of embodiment 366 or 367, wherein the binding of the first expression repressor to the promoter or a sequence proximal thereto and the second expression repressor to the super-enhancer or a sequence proximal thereto decreases viability 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the promoter or a sequence proximal thereto or the binding of the second expression repressor to the super-enhancer or a sequence proximal thereto individually, e.g., as measured by CellTiter Glo.
   369. The system of any of embodiments 329-368, wherein, administration of the first expression repressor and the second expression repressor result in apoptosis of at least 5%, 6%, 7%, 8%, 9% 10%, 12%, 15%, 17% 20%, 25% 30%, 40%, 45%, 50%, 55%, 60%, 65%, 75% of target cells (e.g., cancer cells).
   370. The system of embodiments 329-369, wherein the plurality of cells comprises a plurality of cancer cells and a plurality of non-cancer cells.
   371. The system of embodiment 370, wherein contacting the plurality of cells with the system or a nucleic acid encoding the system decreases the viability of the plurality of cancer cells more than it decreases the viability of the plurality of non-cancer cells.
   372. The system of embodiment 370 or 371, wherein contacting the plurality of cells with the system or a nucleic acid encoding the system decreases the viability of the plurality of cancer cells 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than it decreases the viability of the plurality of non-cancer cells.
   373. The expression repressor or system of any preceding embodiments, which does not reduce viability of non-cancer cells (e.g., primary hepatocytes) by more than 5, 10, 15, or 20%, e.g., when assayed according to Example 29.
   374. The expression repressor or system of embodiment 320, wherein viability is assayed 72 hours after contacting the cells with the expression repressor or system.
   375. The expression repressor or system of embodiment 374, wherein the assay comprises contacting the non-cancer cells with 2.5, 2, 1.25, 1, 0.6, or 0.5 ug/ml of the expression repressor or system.
   376. The system of any of embodiments 352-375, which, when contacted with a plurality of infected cells and a plurality of uninfected cells, decreases the viability of the plurality of infected cells more than it decreases the viability of the plurality of uninfected cells and/or decreases the viability of the plurality of cancerous cells more than it decreases the viability of the plurality of non-cancerous cells.
   377. The system of any of embodiments 352-376, wherein the cancer is hepatocellular carcinoma (HCC), Fibrolamellar Hepatocellular Carcinoma (FHCC), Cholangiocarcinoma, Angiosarcoma, secondary liver cancer, Non-small cell lung cancer (NSCLC), Adenocarcinoma, Small cell lung cancer (SCLC), Large cell (undifferentiated) carcinoma, triple negative breast cancer, gastric adenocarcinoma, endometrial carcinoma, or pancreatic carcinoma.
   378. The system of any of embodiments 352-377, wherein the cancer cells are lung cancer cells, gastric cancer cells, gastrointestinal cancer cells, colorectal cancer cells, pancreatic cancer cells, or hepatic cancer cells.
   379. The system of any of embodiments 352-378, wherein the cells are human lung epithelial cells or human lung fibroblast cells
   380. The system of any of embodiments 352-379, wherein the infection is viral.
   381. The expression repressor of embodiment 380, wherein the viral infection is hepatitis, e.g., hepatitis B.
   382. The system of any of embodiments 378-381, wherein the infected cells are human hepatocytes.
   383. The system of any of embodiments 352-382, wherein the viral infection is a chronic infection.
   384. A fusion protein comprising:
a first amino acid region comprising a sequence encoding the first expression repressor of a system of any of embodiments 118-383; and
a second amino acid region comprising a sequence encoding the second expression repressor of a system of any of embodiments 118-383.
   385. The fusion protein of embodiment 384 which comprises a third amino acid region, wherein the third amino acid region is situated between the first amino acid region and the second amino acid region.
   386. The fusion protein of embodiment 385, wherein the third amino acid region comprises a protease cleavage peptide sequence, e.g., a self-cleaving peptide sequence, e.g., a T2A self-cleaving peptide sequence, e.g., a sequence according to SEQ ID NO: 120.
   387. The fusion protein of embodiment 386, wherein the third amino acid region comprises a protease cleavage peptide sequence, e.g., a self-cleaving peptide sequence, e.g., a tandem 2A peptide sequence, e.g., a tPT2A sequence, e.g., a sequence according to SEQ ID NO: 124.
   388. The fusion protein of embodiment 385, wherein the peptide sequence comprises a T2A peptide sequence and a P2A peptide sequence.
   389. The fusion protein of any of embodiments 384-388, wherein:
the first expression repressor comprises an amino acid sequence according to SEQ ID NO: 30 or 129, or a sequence with at least 80, 85, 90, 95, or 99% identity thereto; and
the second expression repressor comprises an amino acid sequence according to SEQ ID NO: 24 or 142, or a sequence with at least 80, 85, 90, 95, or 99% identity thereto.
   390. The fusion protein of any of embodiments 384-388, wherein: the first expression repressor comprises an amino acid sequence according to SEQ ID NO: 30 or 129, or a sequence with at least 80, 85, 90, 95, or 99% identity thereto; and the second expression repressor comprises an amino acid sequence according to SEQ ID NO: 177 or 183, or a sequence with at least 80, 85, 90, 95, or 99% identity thereto.
   391. The fusion protein of any of embodiments 384-388, wherein:
the first expression repressor comprises an amino acid sequence according to SEQ ID NO: 30 or 129, or a sequence with at least 80, 85, 90, 95, or 99% identity thereto; and
the second expression repressor comprises an amino acid sequence according to SEQ ID NO: 179 or 185, or a sequence with at least 80, 85, 90, 95, or 99% identity thereto.
   392. The fusion protein of any of embodiments 384-391, which comprises an amino acid sequence of SEQ ID NO: 91, 92, 121, or 122, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   393. The fusion protein of any of embodiments 384-392, which comprises an amino acid sequence of SEQ ID NO: 181, 182, 187, or 188, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   394. A nucleic acid comprising a sequence encoding the system of any of embodiments 118-393.
   395. A nucleic acid comprising a sequence encoding the system of embodiment 394.
   396. The nucleic acid of embodiment 394 or 395, which comprises:
a first region comprising a sequence encoding the first expression repressor of a system of any of embodiments 118-393; and
a second region comprising a sequence encoding the second expression repressor of a system of any of embodiments 118-393.
   397. The nucleic acid of any of embodiments 394-396, which comprises a third region, wherein the third region is situated between the first region and the second region.
   398. The nucleic acid of any of embodiments 394-397, wherein the third region encodes a ribosome-skipping sequence.
   399. The nucleic acid of embodiment 397 or 398, wherein the third region encodes a tPT2A peptide sequence, e.g., a sequence according to SEQ ID NO: 124.
   400. The nucleic acid of any of embodiments 397-399, wherein the third region encodes a protease cleavage peptide sequence, e.g., a self-cleaving peptide sequence, e.g., a T2A self-cleaving peptide sequence, e.g., a sequence according to SEQ ID NO: 95.
   401. The nucleic acid of any of embodiments 397-400, wherein the third region encodes a protease cleavage peptide sequence, e.g., a self-cleaving peptide sequence, e.g., a tandem 2A peptide sequence, e.g., a tPT2A peptide sequence, e.g., a sequence according to SEQ ID NO: 124.
   402. The nucleic acid of any of embodiments 394-401, wherein the first expression repressor comprises an amino acid sequence according to SEQ ID NO: 30, 129 or a sequence with at least 80, 85, 90, 95, or 99% identity thereto; and the second expression repressor comprises an amino acid sequence according to SEQ ID NO: 24, 142, or a sequence with at least 80, 85, 90, 95, or 99% identity thereto.
   403. The nucleic acid of any of embodiments 394-401, wherein the first expression repressor comprises an amino acid sequence according to SEQ ID NO: 30, 129 or a sequence with at least 80, 85, 90, 95, or 99% identity thereto; and the second expression repressor comprises an amino acid sequence according to SEQ ID NO: 177, 179, 183, or 185 or a sequence with at least 80, 85, 90, 95, or 99% identity thereto.
   404. The nucleic acid of any of embodiments 394-403, which encodes an amino acid sequence of SEQ ID NO: 91, 92, 121, 122 or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   405. The nucleic acid of any of embodiments 394-404, which encodes an amino acid sequence of SEQ ID NO: 181, 182, 187, 188, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   406. The nucleic acid of any of embodiments 394-405, which comprises a nucleotide sequence of SEQ ID NO: 93, 94, 112, or 113 or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   407. The nucleic acid of any of embodiments 394-406, which comprises a nucleotide sequence of SEQ ID NO: 196, 197, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   408. A nucleic acid comprising a sequence encoding the expression repressor or the expression repressor system of any of embodiments 1-407.
   409. The nucleic acid of any of embodiments 394-408, which is an RNA, e.g., an mRNA.
   410. The nucleic acid of any of embodiments 394-409, which comprises an N7-methylated guanosine, e.g., linked to the 5′ end of the RNA, e.g., via a reverse 5′ to 5′ triphosphate linkage.
   411. The nucleic acid of any of embodiments 394-410, which comprises a 5′ UTR.
   412. The nucleic acid of any of embodiments 394-411, which comprises a Kozak sequence, e.g., between the 5′ UTR and the sequence encoding the expression repressor.
   413. A system comprising:
a first nucleic acid comprising a sequence encoding the first expression repressor of a system of any of embodiments 118-393; and
a second nucleic acid comprising a sequence encoding a second expression repressor, e.g., the second expression repressor of a system of any of embodiments 118-393.
   414. The system of embodiment 413, wherein the first nucleic acid has a nucleotide sequence of SEQ ID NO: 63, 130, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second nucleic acid having a nucleotide sequence of SEQ ID NO: 57, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   415. The system of embodiment 414, wherein the first nucleic acid has a nucleotide sequence of SEQ ID NO: 63, 130, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second nucleic acid having a nucleotide sequence of SEQ ID NO: 189, or 194, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   416. The system of embodiment 415, wherein the first nucleic acid has a nucleotide sequence of SEQ ID NO:189, 194, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second nucleic acid having a nucleotide sequence of SEQ ID NO: 63, 130, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
   417. The nucleic acid or system of any of embodiments 394-416, wherein the nucleic acid comprises mRNA.
   418. A vector comprising the nucleic acid encoding the system, or expression repressor of any of the preceding embodiments.
   419. A lipid nanoparticle comprising the system, nucleic acid, mRNA, or vector of any of the preceding embodiments.
   420. The lipid nanoparticle of embodiment 419 comprising an ionizable lipid, e.g., a cationic lipid, e.g., MC3, SSOP.
   421. The lipid nanoparticle of embodiment 419 or 420, further comprising one or more of neutral lipids, ionizable amine-containing lipids, biodegradable alkyn lipids, steroids, phospholipids, polyunsaturated lipids, structural lipids (e.g., sterols), PEG, cholesterol, or polymer conjugated lipids.
   422. A reaction mixture comprising the expression repressor, system, nucleic acid, vector, or lipid nanoparticle of any of the preceding embodiments.
   423. The reaction mixture of embodiment 422, further comprising a cell.
   424. A pharmaceutical composition comprising the expression repressor, system, nucleic acid, vector, lipid nanoparticle or the reaction mixture of any preceding embodiments.
   425. A method of decreasing expression of a MYC gene in a cell, the method comprising:
contacting the cell (e.g., a cancer cell) with an expression repressor, a system, one or more nucleic acids encoding said system or expression repressor, a vector, a lipid nanoparticle, or a pharmaceutical composition of any of embodiments 1-424,
thereby decreasing expression of the MYC gene in the cell.
   426. A method of treating cancer in a subject in need thereof, the method comprising:
administering the expression repressor, system, nucleic acid, vector, lipid nanoparticle, or a pharmaceutical composition of any of embodiments 1-424 to the subject,
thereby treating the cancer in the subject.
   427. A method of reducing tumor growth in a subject in need thereof, the method comprising:
administering the expression repressor, system, nucleic acid, vector, lipid nanoparticle, or a pharmaceutical composition of any of embodiments 1-424 to the subject,
thereby reducing the tumor size in the subject.
   428. The method of embodiment 427, wherein the reduction in tumor growth comprises reduction of tumor volume compared to tumor volume at the start of treatment.
   429. The method of embodiment 428, wherein the reduction in tumor growth in the subject is greater compared to an untreated subject.
   430. A method of increasing or restoring sensitivity of a cancer to a kinase inhibitor, e.g., sorafenib, the method comprising administering an expression repressor or system described herein to a subject having the cancer.
   431. The method of embodiment 430, wherein administration of the expression repressor or system lowers the IC₅₀ of the kinase inhibitor by 10%, 20%, 30%, or 40%, e.g., in a cancer cell viability assay, e.g., an assay according to Example 38.
   432. The method of embodiment 430 or 431, wherein the kinase inhibitor inhibits one or more of (e.g., all of) VEGFR, PDGFR, or RAF kinase.
   433. A method of increasing or restoring sensitivity of a cancer to a bromodomain inhibitor, e.g., a BET inhibitor, e.g., JQ1, the method comprising administering an expression repressor, system, or nucleic acid described herein (e.g., of any of embodiments 1-423) to a subject having the cancer, wherein optionally administration of the expression repressor or system lowers the IC₅₀ of the bromodomain inhibitor by 10%, 20%, 30%, 40%, 50%, 60%, 70%, 80%, 90%, or 95%, e.g., in a cancer cell viability assay, e.g., an assay according to Example 39.
   434. The method of embodiment 433 wherein the bromodomain inhibitor is or comprises JQ1, BET672, or birabresib.
   435. A method of increasing or restoring sensitivity of a cancer to a MEK inhibitor, e.g., Trametinib, the method comprising administering an expression repressor, system, or nucleic acid described herein (e.g., of any of embodiments 1-423) to a subject having the cancer, wherein optionally administration of the expression repressor or system lowers the IC₅₀ of the MEK inhibitor by 10%, 20%, 30%, 40%, 50%, 60%, 70%, 80%, 90%, or 95%, e.g., in a cancer cell viability assay, e.g., an assay according to Example 51.
   436. The method of any of embodiments 427-435, wherein the reduction in tumor growth in the subject is greater than or similar to a tumor size reduction when the subject is treated with a chemotherapeutic agent or small molecule MYC inhibitor.
   437. The method of embodiment 436, wherein the chemotherapeutic agent is sorafenib or cisplatin.
   438. The method of embodiment 437, wherein the small molecule MYC inhibitor is MYCi975.
   439. The method of reducing tumor size in a subject in need thereof, the method comprising:
administering the expression repressor, system, nucleic acid, vector, lipid nanoparticle, or a pharmaceutical composition of 1-424 to the subject, wherein the reduction in tumor size is greater than or similar to a tumor size reduction when the subject is treated with a chemotherapeutic agent.
   440. The method of 439 wherein the chemotherapeutic agent is sorafenib or cisplatin.
   441. The method of any of the preceding embodiments wherein the subject does not experience any significant side effects compared to when treated with a chemotherapeutic agent or a small molecule MYC inhibitor.
   442. The method of any of embodiments 436-441, wherein the chemotherapeutic agent is sorafenib or cisplatin.
   443. The method of embodiment 442, wherein the small molecule MYC inhibitor is MYCi975.
   444. The method of any of embodiments 426-443, wherein the cancer is stage I, stage II, stage III, or stage IV cancer.
   445. The method of any of preceding embodiments wherein the subject's body weight remains about the same before treatment and post-treatment.
   446. The method of any of preceding embodiments, wherein the subject does not experience a decrease in body weight, or wherein the subject experiences a decrease in body weight of less than 3%, 2%, or 1% compared to at the start of treatment.
   447. The method of any of the preceding embodiments wherein the subject does not experience a reduction or gain in body weight post-treatment compared to the subject's body weight before the treatment.
   448. A method of treating a liver disease in a subject in need thereof, the method comprising:
administering an expression repressor to the subject, wherein the expression repressor comprises targeting moiety that binds a MYC locus (e.g., a transcribed region of MYC, a MYC promoter, or an anchor sequence of an anchor sequence mediated conjunction (ASMC) comprising a MYC gene or to a sequence proximal to the anchor sequence), and optionally, an effector moiety, e.g., an effector moiety described herein;
thereby treating the liver disease in the subject.
   449. The method of embodiment 447, which further comprises administering to the subject a second expression repressor, the second expression repressor comprising a targeting moiety that binds to an anchor sequence of an anchor sequence mediated conjunction (ASMC) comprising a target gene, e.g., MYC, and optionally, a second effector moiety, e.g., an effector moiety described herein; e.g., KRAB;
thereby treating the liver disease in the subject.
   450. A method of treating a liver disease in a subject in need thereof, the method comprising:
administering the expression repressor, system, nucleic acid, vector, lipid nanoparticle, or a pharmaceutical composition, of any of embodiments 1-424 to the subject,
thereby treating the liver disease in the subject.
   451. The method of embodiment 450, wherein the liver disease is a chronic liver disease.
   452. The method of embodiment 450 or 451 wherein the liver disease is viral or alcohol related.
   453. The method of any of embodiments 450-452, wherein the liver disease is hepatitis or hepatocellular carcinoma.
   454. The method of embodiment 453, wherein the hepatocellular carcinoma is selected from HCC subtype S1, HCC subtype S2, or HCC subtype S3.
   455. The method of embodiment 453 or 454, wherein the hepatocellular carcinoma is HCC S1.
   456. The method of embodiment 453 or 454, wherein the hepatocellular carcinoma is HCC S2.
   457. The method of any of embodiments 450-456 where the liver disease is caused by a hepatitis B virus or hepatitis C virus.
   458. A method of treating a pulmonary disease in a subject in need thereof, the method comprising:
administering an expression repressor to the subject, wherein the expression repressor comprises targeting moiety that binds a MYC locus (e.g., a transcribed region of MYC, a MYC promoter, or an anchor sequence of an anchor sequence mediated conjunction (ASMC) comprising a MYC gene or to a sequence proximal to the anchor sequence), and optionally, an effector moiety, e.g., an effector moiety described herein;
thereby treating the pulmonary disease in the subject.
   459. The method of embodiment 458, which further comprises administering to the subject a second expression repressor, the second expression repressor comprising a targeting moiety that binds a genomic locus located in a super enhancer region of a target gene, e.g., MYC, and optionally, a second effector moiety, e.g., an effector moiety described herein; e.g., KRAB;
thereby treating the pulmonary disease in the subject
   460. A method of treating a pulmonary disease in a subject in need thereof, the method comprising:
administering the expression repressor, system, nucleic acid, vector, lipid nanoparticle, or a pharmaceutical composition, of any of embodiments 1-424 to the subject,
thereby treating the pulmonary disease in the subject.
   461. The method of embodiment 459 or 460 where the pulmonary disease is a cancer, e.g., a lung cancer, e.g., a lung carcinoma, e.g., non-small cell lung carcinoma or small cell lung carcinoma.
   462. The method of any of embodiments 425-461, wherein contacting or administering comprises intravenous administration to a subject.
   463. The method of any of embodiments 425-462, wherein contacting or administering comprises intratumoral delivery (e.g., injection).
   464. The method of any of embodiments 425-463, wherein the cancer is characterized by increased MYC expression relative to a reference level (e.g., relative to a reference cell's MYC expression, e.g., an otherwise similar non-cancerous cell of the subject).
   465. The method of any of embodiments 426-464, wherein the cancer is characterized by duplication of a portion of or all of a MYC gene.
   466. The method of any of embodiments 426-465, wherein the cancer is selected from colorectal cancer, breast cancer, AML, prostate cancer, neuroblastoma, lung cancer, endometrial cancer, liver cancer, a lymphoma (e.g., Burkitt lymphoma), carcinoma of the cervix, or stomach cancer.
   467. The method of any of embodiments 426-466, wherein the cancer is a human chorionic gonadotropin (hCG) secreting cancer.
   468. The method of any of embodiments 426-467, wherein the cancer is hepatocarcinoma.
   469. The method of any of embodiments 426-468, wherein the cancer is a non-responsive cancer, e.g., a non-responsive hepatocarcinoma.
   470. The method of any of embodiments 426-469, wherein the cancer is non-small cell lung carcinoma or small cell lung carcinoma.
   471. The method of any of embodiments 426-470, wherein the cancer over-expresses alpha-fetoprotein (AFP) (e.g., relative to a reference cell's AFP expression, e.g., an otherwise similar non-cancerous cell of the subject).
   472. The method of any of embodiments 431-471, wherein cells of the cancer are characterized by the presence of a super enhancer, e.g., comprising the MYC gene or comprising the anchor-sequence mediated conjunction comprising the MYC gene, wherein optionally the cancer is selected from liver cancer, colorectal cancer, breast cancer, AML, prostate cancer, neuroblastoma, lung cancer, or endometrial cancer.
   472. The method of embodiment 471, wherein the expression repressor (e.g., the second expression repressor) binds to an anchor sequence of an anchor sequence mediated conjunction (ASMC) comprising a MYC gene or to a sequence proximal to the anchor sequence.
   473. The method of any of embodiments 426-472, wherein cells of the cancer are characterized by the absence of a super enhancer comprising the MYC gene or comprising the anchor-sequence mediated conjunction comprising the MYC gene.
   474. The method of embodiment 473, wherein the expression repressor (e.g., the first expression repressor) binds the MYC promoter.
   475. The method of any of embodiments 426-474, wherein the cancer comprises cells comprising a super enhancer comprising the MYC gene or comprising the anchor-sequence mediated conjunction comprising the MYC gene, and cells not comprising a super enhancer comprising the MYC gene or comprising the anchor-sequence mediated conjunction comprising the MYC gene.
   476 The method of any of embodiments 426-475, wherein the cancer comprises cells characterized by increased MYC expression relative to a reference level (e.g., relative to a reference cell's MYC expression, e.g., an otherwise similar non-cancerous cell of the subject), and cells not characterized by increased MYC expression relative to a reference level (e.g., relative to a reference cell's MYC expression, e.g., an otherwise similar non-cancerous cell of the subject), e.g., having normal MYC expression.
   477. The method of any of embodiments 426-476, wherein the expression repressor, system, nucleic acid, vector, lipid nanoparticle, or a pharmaceutical composition is administered a monotherapy.
   478. The method of any of embodiments 426-477, which comprises administering a plurality of doses of the expression repressor, system, nucleic acid, vector, lipid nanoparticle, or a pharmaceutical composition to the subject, e.g., at least 2, 3, 4, 5, or 6 doses.
   479. The method of any of embodiments 426-479, which comprises administering a plurality of doses of the expression repressor, system, nucleic acid, vector, lipid nanoparticle, or a pharmaceutical composition to the subject in 5 day intervals.
   480. The method of any of embodiments 426-479, comprising:
a) first, administering to the subject a first plurality of doses of an expression repressor or system described herein (e.g., of any of embodiments 1-424), wherein optionally each subsequent dose in the first plurality is administered 5 days after the previous dose in the first plurality;
b) second, withdrawing the expression repressor or system for a period of time (a “drug holiday”), e.g., for about 2 weeks), and
c) third, administering to the subject a second plurality of doses of the expression repressor or system, wherein optionally a subsequent dose of the second plurality is administered 5 days after the previous dose in the second plurality.
   481. The method of embodiment 480, wherein the first plurality of doses comprises 4 doses.
   482. The method of embodiment 479 or 480, wherein the second plurality of doses comprises 2 doses.
   483. The method of any of embodiments 480-482, wherein the subject receives no therapeutic at all during the drug holiday.
   484. The method of any of embodiments 480-483, wherein the subject receives a second therapeutic agent during the drug holiday.
   485. The method of any of embodiments 480-484, wherein the drug holiday is at least twice as long as the time between administration of doses in the first plurality of doses.
   486. The method of any of embodiments 480-486, wherein the drug holiday is at least twice as long as the time between administration of doses in the second plurality of doses.
   487. The method of any of embodiments 426-486, wherein volume of the tumor declines to undetectable levels following treatment with the expression repressor or system.
   488. The method of any of embodiments 426-487, tumor volume declines (e.g., to undetectable levels) after cessation of treatment with the expression repressor or system.
   489. The method of any of embodiments 425-488, wherein the cancer does not become resistant to the expression repressor or system, or does not become resistant to the expression repressor or system within a period of 10, 20, 30, 40, 50, or 60 days.
   490. The method of any of embodiments 425-489, wherein the cancer cells have a functional apoptotic pathway.
   491. The method of any of embodiments 425-490, wherein the cancer cells have functional Caspase 3.
   492. The method of embodiment 491, wherein Caspase 3 is upregulated in cancer cells upon administration of the expression repressor or system to the subject.
   493. The method of any of embodiments 425-492, wherein Ki67 is downregulated in cancer cells upon administration of the expression repressor or system to the subject.
   494. The method of any of embodiments 425-493, wherein cancer cell proliferation declines upon administration of the expression repressor or system to the subject.
   495. The method of any of embodiments 425-494, wherein the method further comprises
a. contacting the cell with a second therapeutic agent or
b. administering a second therapeutic agent to the subject.
   496. The method of embodiment 495, wherein the second therapeutic agent is not an expression repressor binding to MYC promoter.
   497. The method of embodiment 495 or 496 wherein the second therapeutic agent is not an expression repressor, system, fusion protein, nucleic acid, vector, reaction mixture, pharmaceutical composition, or lipid nanoparticle of any of embodiments 1-424.
   498. The method of any of embodiments 494-496, wherein the second therapeutic agent is the expression repressor, system, fusion protein, nucleic acid, vector, reaction mixture, pharmaceutical composition, or lipid nanoparticle, of any of embodiments 1-424.
   499. The method of any of embodiments 495-497, wherein the second therapeutic agent is an immunotherapy, one or both of immune checkpoint and anti-vascular-endothelial-growth-factor therapy, systemic chemotherapy, a tyrosine kinase inhibitor, e.g., sorafenib, a mitogen-activated protein kinase kinase inhibitor (MEK inhibitor), e.g., trametinib, or a bromodomain inhibitor, e.g., a BET inhibitor, e.g., JQ1 or birabresib.
   500. The method of any of embodiments 495-499, wherein the second therapeutic agent is a tyrosine kinase inhibitor, e.g., sorafenib.
   501. The method of any of embodiments 495-499, wherein the second therapeutic agent is a bromodomain inhibitor, e.g., a BET inhibitor, e.g., JQ1, birabresib, or BET 672.
   502. The method of any of embodiments 495-499, wherein the second therapeutic agent is a mitogen-activated protein kinase kinase inhibitor (MEK inhibitor), e.g., trametinib.
   503. The method of any of embodiments 495-502, wherein the method further comprises administering an additional therapy to the subject.
   504. The method of embodiment 504, wherein the additional therapy comprises surgical resection orthotopic liver transplantation, radiofrequency ablation, photodynamic therapy (PDT), laser therapy, brachytherapy, radiation therapy, trans-catheter arterial chemo- or radio-embolization, or stereotactic radiation therapy.
   505. The method of any of embodiments 495-504, wherein the second therapeutic agent is selected from a checkpoint inhibitor or a small molecule.
   506. The method of any of embodiments 495-505, wherein the second therapeutic agent is a chemotherapeutic agent, e.g., a kinase inhibitor or a bromodomain inhibitor, e.g., a BET inhibitor.
   507. The method of embodiments 505 or 506 wherein the second therapeutic agent is selected from sorafenib, JQ1, BET672, birabresib, or trametinib.
   508. The method of any of embodiments 495-506, wherein the expression repressor, system, or nucleic acid and the second therapeutic agent are administered concurrently.
   509. The method of any of embodiments 495-508, wherein the expression repressor, system, or nucleic acid and the second therapeutic agent are administered sequentially.
   509. The method of any of embodiments 503-509, wherein the additional therapy is administered concurrently.
   511. The method of any of embodiments 503-510, wherein the additional therapy is administered sequentially.
   519. The method of any of embodiments 495-511, wherein second therapeutic agent is administered simultaneously with the expression repressor, system, nucleic acid, vector, lipid nanoparticle, pharmaceutical composition, or reaction mixture of any of embodiments 1-424.
   513. The method of any of embodiments 495-512, wherein second therapeutic agent is administered consecutively with the expression repressor, system, nucleic acid, vector, lipid nanoparticle, pharmaceutical composition, or reaction mixture of any of embodiments 1-424.
   514. The method of any of embodiments 495-513, wherein the expression repressor, system, or nucleic acid is administered intravenously, and the second therapy is administered orally.
   515. The method of any of the preceding embodiments, wherein the cancer is a resistant or refractory cancer.
   516. The method of any of the preceding embodiments, wherein the cancer is resistant or refractory to a kinase inhibitor, e.g., a kinase inhibitor that inhibits one or more of VEGFR, PDGFR, or RAF kinase, e.g., sorafenib.
   517. The method of any of the preceding embodiments, wherein the subject has an amplification in the MYC super-enhancer.
   518. A kit comprising a container comprising a composition comprising an expression repressor, a system, one or more nucleic acids encoding said system or expression repressor, a vector, a lipid nanoparticle, reaction mixture, or a pharmaceutical composition of any of embodiments 1-424 and a set of instructions comprising at least one method for modulating, e.g., decreasing the expression of a MYC gene within a cell with said composition.

### Definitions

A, an, the: As used herein, the singular forms “a,” “an” and “the” include plural referents unless the context clearly dictates otherwise.

Agent: As used herein, the term “agent”, may be used to refer to a compound or entity of any chemical class including, for example, a polypeptide, nucleic acid, saccharide, lipid, small molecule, metal, or combination or complex thereof. As will be clear from context to those skilled in the art, in some embodiments, the term may be utilized to refer to an entity that is or comprises a cell or organism, or a fraction, extract, or component thereof. Alternatively, or additionally, as those skilled in the art will understand in light of context, in some embodiments, the term may be used to refer to a natural product in that it is found in and/or is obtained from nature. In some embodiments, again as will be understood by those skilled in the art in light of context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some embodiments, the term “agent” may refer to a compound or entity that is or comprises a polymer; in some embodiments, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Anchor Sequence: The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present disclosure, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated. In some embodiments, the anchor sequence comprises or is a nucleating polypeptide binding motif. In some embodiments, the anchor sequence is adjacent to a nucleating polypeptide binding motif.

Anchor Sequence-Mediated Conjunction: The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences (see, e.g. FIG. 1).

Associated with: Two events or entities are “associated” with one another, as that term is used herein, if presence, level, form and/or function of one is correlated with that of the other. For example, in some embodiments, a particular entity (e.g., polypeptide, genetic signature, metabolite, microbe, etc.) is considered to be associated with a particular disease, disorder, or condition, if its presence, level, form and/or function correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a DNA sequence is “associated with” a target genomic or transcription complex when the nucleic acid is at least partially within the target genomic or transcription complex, and expression of a gene in the DNA sequence is affected by formation or disruption of the target genomic or transcription complex.

Domain: As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).

Effector moiety: As used herein, the term “effector moiety” refers to a domain that is capable of altering the expression of a target gene when localized to an appropriate site in the nucleus of a cell. In some embodiments, an effector moiety recruits components of the transcription machinery. In some embodiments, an effector moiety inhibits recruitment of components of transcription factors or expression repressing factors. In some embodiments, an effector moiety comprises an epigenetic modifying moiety (e.g., epigenetically modifies a target DNA sequence).

Epigenetic modifying moiety: As used herein, “epigenetic modifying moiety” refers to a domain that alters: i) the structure, e.g., two dimensional structure, of chromatin; and/or ii) an epigenetic marker (e.g., one or more of DNA methylation, histone methylation, histone acetylation, histone sumoylation, histone phosphorylation, and RNA-associated silencing), when the epigenetic modifying moiety is appropriately localized to a nucleic acid (e.g., by a targeting moiety). In some embodiments, an epigenetic modifying moiety comprises an enzyme, or a functional fragment or variant thereof, that affects (e.g., increases or decreases the level of) one or more epigenetic markers. In some embodiments, an epigenetic modifying moiety comprises a DNA methyltransferase, a histone methyltransferase, CREB-binding protein (CBP), or a functional fragment of any thereof.

Expression control sequence: As used herein, the term “expression control sequence” refers to a nucleic acid sequence that increases or decreases transcription of a gene and includes (but is not limited to) a promoter and an enhancer. An “enhancing sequence” refers to a subtype of expression control sequence and increases the likelihood of gene transcription. A “silencing or repressor sequence” refers to a subtype of expression control sequence and decreases the likelihood of gene transcription.

Expression repressor: As used herein, the term “expression repressor” refers to an agent or entity with one or more functionalities that decreases expression of a target gene in a cell and that specifically binds to a DNA sequence (e.g., a DNA sequence associated with a target gene or a transcription control element operably linked to a target gene). An expression repressor comprises at least one targeting moiety and optionally one effector moiety.

Expression repression system: As used herein, the term “expression repression system” refers to a plurality of expression repressors which decrease expression of a target gene in a cell. In some embodiments, an expression repression system comprises a first expression repressor and a second expression repressor, wherein the first expression repressor and second expression repressor (or nucleic acids encoding the first expression repressor and second expression repressor) are present together in a single composition, mixture, or pharmaceutical composition. In some embodiments, an expression repression system comprises a first expression repressor and a second expression repressor, wherein the first expression repressor and second expression repressor (or nucleic acids encoding the first expression repressor and second expression repressor) are present in separate compositions or pharmaceutical compositions. In some embodiments, the first expression repressor and the second expression repressor are present in the same cell at the same time. In some embodiments, the first expression repressor and the second expression repressor are not present in the same cell at the same time, e.g., they are present sequentially. For example, the first expression repressor may be present in a cell for a first time period, and then the second expression repressor may be present in the cell for a second time period, wherein the first and second time periods may be overlapping or non-overlapping.

Fusion Molecule: As used herein, the term “fusion molecule” refers to a compound comprising two or more moieties, e.g., a targeting moiety and an effector moiety, that are covalently linked. A fusion molecule and its moieties may comprise any combination of polypeptide, nucleic acid, glycan, small molecule, or other components described herein (e.g., a targeting moiety may comprise a nucleic acid and an effector moiety may comprise a polypeptide). In some embodiments, a fusion molecule is a fusion protein, e.g., comprising one or more polypeptide domains covalently linked via peptide bonds. In some embodiments, a fusion molecule is a conjugate molecule that comprises a targeting moiety and effector moiety that are linked by a covalent bond other than a peptide bond or phosphodiester bond (e.g., a targeting moiety that comprises a nucleic acid and an effector moiety comprising a polypeptide linked by a covalent bond other than a peptide bond or phosphodiester bond). In some embodiments, an expression repressor is or comprises a fusion molecule.

Genomic complex: As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex binds. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that it does not adopt when the complex is not formed.

Moiety: As used herein, the term “moiety” refers to a defined chemical group or entity with a particular structure and/or or activity, as described herein.

Modulating agent: As used herein, the term “modulating agent” refers to an agent comprising one or more targeting moieties and one or more effector moieties that is capable of altering (e.g., increasing or decreasing) expression of a target gene, e.g., MYC.

MYC: As used herein, the terms “MYC locus” refer to the portion of the human genome that encodes a MYC polypeptide (e.g., the polypeptide disclosed in NCBI Accession Number NP002458.2, or a mutant thereof), the promoter operably linked to MYC (“MYC promoter”), and the anchor sequences that form an ASMC comprising the MYC gene. In some embodiments, the MYC locus encodes a nucleic acid having NCBI Accession Number NM-002467. In some embodiments, the MYC gene is a proto-oncogene, and in some embodiments the MYC gene is an oncogene. In certain instances, a MYC gene is found on chromosome 8, at 8q24.21. In certain instances, a MYC gene begins at 128,816,862 bp from pter and ends at 128,822,856 bp from pter. In certain instances, a MYC gene is about 6 kb. In certain instances, a MYC gene encodes at least eight separate mRNA sequences—5 alternatively spliced variants and 3 un-spliced variants.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Nucleating polypeptide: As used herein, the term “nucleating polypeptide” or “conjunction nucleating polypeptide” as used herein, refers to a protein that associates with an anchor sequence directly or indirectly and may interact with one or more conjunction nucleating polypeptides (that may interact with an anchor sequence or other nucleic acids) to form a dimer (or higher order structure) comprised of two or more such conjunction nucleating polypeptides, which may or may not be identical to one another. When conjunction nucleating polypeptides associated with different anchor sequences associate with each other so that the different anchor sequences are maintained in physical proximity with one another, the structure generated thereby is an anchor-sequence-mediated conjunction. That is, the close physical proximity of a nucleating polypeptide-anchor sequence interacting with another nucleating polypeptide-anchor sequence generates an anchor sequence-mediated conjunction (e.g., in some cases, a DNA loop), that begins and ends at the anchor sequence. As those skilled in the art, reading the present specification will immediately appreciate, terms such as “nucleating polypeptide”, “nucleating molecule”, “nucleating protein”, “conjunction nucleating protein”, may sometimes be used to refer to a conjunction nucleating polypeptide. As will similarly be immediately appreciated by those skilled in the art reading the present specification, an assembles collection of two or more conjunction nucleating polypeptides (which may, in some embodiments, include multiple copies of the same agent and/or in some embodiments one or more of each of a plurality of different agents) may be referred to as a “complex”, a “dimer” a “multimer”, etc.

Operably linked: As used herein, the phrase “operably linked” refers to a juxtaposition wherein the components described are in a relationship permitting them to function in their intended manner. A transcription control element “operably linked” to a functional element, e.g., gene, is associated in such a way that expression and/or activity of the functional element, e.g., gene, is achieved under conditions compatible with the transcription control element. In some embodiments, “operably linked” transcription control elements are contiguous (e.g., covalently linked) with coding elements, e.g., genes, of interest, in some embodiments, operably linked transcription control elements act in trans to or otherwise at a distance from the functional element, e.g., gene, of interest. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1000, 500, 100, 50, or 10 base pairs of each other or directly adjacent to each other.

Peptide, Polypeptide, Protein: As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types.

Pharmaceutical composition: As used herein, the term “pharmaceutical composition” refers to an active agent (e.g., a modulating agent, e.g., a disrupting agent), formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; trans-dermally; or nasally, pulmonary, and/or to other mucosal surfaces.

Proximal: As used herein, “proximal” refers to a closeness of two sites, e.g., nucleic acid sites, such that binding of an expression repressor at the first site and/or modification of the first site by an expression repressor will produce the same or substantially the same effect as binding and/or modification of the other site. For example, a targeting moiety may bind to a first site that is proximal to an enhancer (the second site), and the effector moiety associated with said targeting moiety may epigenetically modify the first site such that the enhancer's effect on expression of a target gene is modified, substantially the same as if the second site (the enhancer sequence) had been bound and/or modified. In some embodiments, a site proximal to a target gene (e.g., an exon, intron, or splice site within the target gene), proximal to a transcription control element operably linked to the target gene, or proximal to an anchor sequence is less than 5000, 4000, 3000, 2000, 1000, 900, 800, 700, 600, 500, 400, 300, 200, 100, 50, or 25 base pairs from the target gene (e.g., an exon, intron, or splice site within the target gene), transcription control element, or anchor sequence (and optionally at least 20, 25, 50, 100, 200, or 300 base pairs from the target gene (e.g., an exon, intron, or splice site within the target gene), transcription control element, or anchor sequence).

Specific: As used herein, the term “specific”, referring to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In some embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments, specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specific binding: As used herein, the term “specific binding” refers to an ability to discriminate between possible binding partners in the environment in which binding is to occur. In some embodiments, a binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex. In some embodiments, specific binding is assessed by detecting or determining ability of the binding agent to compete with an alternative interaction between its partner and another entity. In some embodiments, specific binding is assessed by performing such detections or determinations across a range of concentrations.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

Symptoms are reduced: As used herein, the phrase “symptoms are reduced” may be used when one or more symptoms of a particular disease, disorder or condition is reduced in magnitude (e.g., intensity, severity, etc.) and/or frequency. In some embodiments, a delay in the onset of a particular symptom is considered one form of reducing the frequency of that symptom.

Target: An agent or entity is considered to “target” another agent or entity, in accordance with the present disclosure, if it binds specifically to the targeted agent or entity under conditions in which they come into contact with one another. In some embodiments, for example, an antibody (or antigen-binding fragment thereof) targets its cognate epitope or antigen. In some embodiments, a nucleic acid having a particular sequence targets a nucleic acid of substantially complementary sequence.

Target gene: As used herein, the term “target gene” means a gene that is targeted for modulation, e.g., of expression. In some embodiments, a target gene is part of a targeted genomic complex (e.g. a gene that has at least part of its genomic sequence as part of a target genomic complex, e.g. inside an anchor sequence-mediated conjunction), which genomic complex is targeted by one or more modulating agents as described herein. In some embodiments, modulation comprises inhibition of expression of the target gene. In some embodiments, a target gene is modulated by contacting the target gene or a transcription control element operably linked to the target gene with an expression repression system, e.g., expression repressor(s), described herein. In some embodiments, a target gene is aberrantly expressed (e.g., overexpressed) in a cell, e.g., a cell in a subject (e.g., patient).

Targeting moiety: As used herein, the term “targeting moiety” means an agent or entity that specifically targets, e.g., binds, a genomic sequence element (e.g., an expression control sequence or anchor sequence). In some embodiments, the genomic sequence element is proximal to and/or operably linked to a target gene (e.g., MYC).

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent comprises an expression repression system, e.g., an expression repressor, described herein. In some embodiments, a therapeutic agent comprises a nucleic acid encoding an expression repression system, e.g., an expression repressor, described herein. In some embodiments, a therapeutic agent comprises a pharmaceutical composition described herein.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) or tissue(s), etc. For example, in some embodiments, an effective amount of compound in a formulation to treat a disease, disorder, and/or condition is an amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

### BRIEF DESCRIPTION OF THE FIGURES

The following detailed description of the embodiments of the disclosure will be better understood when read in conjunction with the appended drawings. For the purpose of illustrating the disclosure, there are shown in the drawing embodiments, which are presently exemplified. It should be understood, however, that the disclosure is not limited to the precise arrangement and instrumentalities of the embodiments shown in the drawings.

FIG. 1A depicts a schematic representation of a dual target approach based on a durable block of the MYC promoter using a DBD fused to a DNA methyltransferase, and a transient (48/72 Hours) block of CTCF/TF sites using a DBD or a DBD fused to a short-term effector.

FIG. 1B depicts guide RNA localization and chromatin context of target sites (CTCF and promoter) for the MYC gene. From top to bottom, the graphs represent, for the MYC locus in HepG2 cells, H3K4me3 (histone H3 Ky trimethylation) levels; H3K9me3 (histone H3 K9 trimethylation) levels (replicate 1); H3K9me3 (histone H3 K9 trimethylation) levels (replicate 2); H3K27me3 (histone H3 K27 trimethylation) levels; H3K27ac (histone H3 K27 acetylation) levels; GROseq_fwdStrand levels (binding of transcriptionally active RNA pol II on the forward strand); GROseq_revStrand levels (binding of transcriptionally active RNA pol II on the reverse strand); RNAseq_rep2 levels (MYC transcript levels measured using RNAseq, replicate 2); DNA methylation level as measured by WGBS (whole-genome bisulfite sequencing), and CTCF binding levels. The positions of four gRNAs are indicated with arrows. gRNAs GD-28859, GD-28616, GD-28862 target at or near the anchor site upstream of MYC, and gRNA GD-28617 targets the MYC promoter. In this disclosure, GD-28859 is also referred to as GD-59; GD-28616 is also referred to as GD-16; GD-28862 is also referred to as GD-62; and GD-28617 is also referred to as GD-17.

FIG. 1C shows a schematic diagram of an exemplary bi-cistronic construct. The 5′ end of the construct possesses a cap structure defined by an N7-methylated guanosine linked to the first nucleotide of the mRNA via a reverse 5′ to 5′ triphosphate linkage. In some embodiments, the cap structure promotes protein translation and stability. Downstream of the cap structure is an un-translated region (5′ UTR) designed to promote high levels of protein translation, followed by the canonical “Kozak” sequence that is recognized by the ribosome to start translation of the protein. Following the “Kozak” sequence is the CDS which is a single continuous sequence comprising the first expression repressor comprising a first targeting moiety and a first effector moiety and the second expression repressor comprising the second targeting moiety and the second effector moiety separated by a tPT2A “ribosome skipping” sequence (the linker). Without wishing to be bound by theory, when a ribosome reaches the tPT2A linker, it begins translating the linker into amino acids. The first 18 amino acids produced from the P2A linker remain at the C-terminal end of the first expression repressor (e.g., comprising a ZF DBD and MQ1), which the ribosome then releases. The ribosome then continues on until it reaches the T2A linker, and the first 17 residues of the T2A linker are translated and released. Next, the second polypeptide is translated, comprising a single amino acid and then the beginning of the second expression repressor (e.g., comprising a second ZF DBD and KRAB). After the CDS is a 3′ UTR which is designed to aid in high levels of translation and to also stabilize the mRNA. Finally, at the very 3′ end of the mRNA is the polyA tail. In some embodiments, the polyA tail promotes protein translation and mRNA stability.

FIG. 2A shows that Cas9-Nuclease editing of the CTCF motif results in down-regulation of MYC expression. Disruption of the CTCF motif with Cas9 (in combination with GD-28616) resulted in a 32-39% down-regulation in MYC expression in all three HCC cell lines (HepG2, Hep3B and SKHEP1). Disruption of the region adjacent to the CTCF motif (GD-28859) regulated MYC expression 35-45% in two (HepG2 and Hep3B) of the three cell lines.

FIG. 2B shows that editing efficiency as assessed by AmpSeq confirmed 77-100% editing of all the cell lines.

FIG. 3 shows that dCas9-KRAB down-regulates MYC expression when directed to the promotor or associated CTCF motif. LNP-mediated transfection of dCas9-KRAB/GD-28616 down-regulated MYC expression by 11-34% at 48/72-hour timepoints in Hep3B and SKHEP1. LNP-mediated transfection of dCas9-KRAB/GD-28859 down-regulated MYC expression by 18-44% at 48/72-hour timepoints in all 3 HCC models. Directing dCas9-KRAB to the MYC promoter via dCas9-KRAB/GD-28617 down-regulated MYC expression by 24-58% at 48/72-hour timepoints in all 3 HCC models.

FIG. 4A depicts sgRNA localization and zinc finger design at the promoter associated CTCF motif Fig. discloses SEQ ID NO: 208.

FIG. 4B shows that ZF-KRAB constructs directed to the promoter associated CTCF effected MYC down-regulation in Hep3B. ZF2-KRAB, ZF3-KRAB and ZF4-KRAB down-regulated MYC to an equivalent or greater degree than dCas9-KRAB/GD-28859 in Hep3B cells, with ZF3-KRAB having the strongest down-regulatory effect.

FIG. 4C shows that ZF3-No Effector and ZF3-KRAB down-regulated MYC expression in multiple human HCC Models (HepG2, Hep3B, and SKHEP1). ZF3-KRAB was also shown to down-regulate MYC to an equivalent or greater degree than ZF3-No Effector and ZF5-No Effector in the other two HCC models, HepG2 and SKHEP1.

FIG. 4D shows that ZF3-No Effector and ZF3-KRAB demonstrated equivalent effects on MYC expression and viability in Hep3B cells at different time points (24 hours, 72 hours, and 120 hours).

FIG. 5 shows that dCas9-MQ1 down-regulated MYC expression when directed at the MYC promoter in multiple HCC models (HepG2, Hep3B, and SKHEP1).

FIG. 6A depicts sgRNA localization and zinc finger design at the MYC promoter. Fig. discloses SEQ ID NO: 209.

FIG. 6B depicts 6 ZF-MQ1 constructs directed to the MYC promoter that were screened for effects on MYC down-regulation in Hep3B. ZF8-MQ1, ZF9-MQ1 and ZF11-MQ1 down-regulated MYC to the greatest degree in Hep3B cells, with ZF9-KRAB MQ1 having the strongest down-regulatory effect.

FIG. 7A shows that ZF9-MQ1 significantly down-regulated MYC expression and reduces viability in Hep3B compared to ZF12-MQ1.

FIG. 7B shows that ZF9-MQ1 significantly down-regulated MYC expression and reduces viability in HepG2 compared to ZF12-MQ1.

FIG. 7C shows that ZF9-MQ1 significantly down-regulated MYC expression and reduces viability in SKHEP1 compared to ZF12-MQ1.

FIG. 7D shows that ZF9-MQ1 is more efficient in down-regulating MYC expression and reducing viability in Hep3B compared to ZF8-MQ1.

FIG. 7E shows that ZF9-MQ1 is more efficient in down-regulating MYC expression and reducing viability in HepG2 compared to ZF8-MQ1.

FIG. 7F shows that ZF9-MQ1 is more efficient in down-regulating MYC expression and reducing viability in SKHEP1 compared to ZF8-MQ1.

FIG. 7G shows that ZF9-MQ1 significantly down-regulated MYC expression and reduces viability in Hep3B compared to dCas9-MQ1/GD17.

FIG. 7H shows that ZF9-MQ1 significantly down-regulated MYC expression and reduces viability in HepG2 compared to dCas9-MQ1/GD17.

FIG. 7I shows that ZF9-MQ1 significantly down-regulated MYC expression and reduces viability in SKHEP1 compared to dCas9-MQ1/GD17.

FIG. 8A shows that dCas9-MQ1 effected a 50-90% decrease in mRNA at 72 hours across the three cell lines (Hep3B, HepG2, and SKHEP1).

FIG. 8B shows that MYC down-regulation dramatically decreased viability in HepG2 and Hep3B at 72 and 168 hours, although SK-HEP-1 viability was minimally affected by MYC down-regulation.

FIG. 8C shows that at day 7 and day 11, MYC mRNA was decreased ˜70% and ˜55% respectively. As far out as day 15, an ˜40% down-regulation in transcript was maintained.

FIG. 8D shows that that treatment with dCas9-MQ1/GD-28617 directs de novo methylation to the targeted region and that these transcriptional changes tightly correlate with the percentage of CpG methylation in the target region and confirmed methylation persists out to day 15.

FIG. 9 shows that treatment with dCas9-MQ1/GD-17 inhibited tumor growth in vivo.

FIG. 10 shows that dCas9-MQ1/GD-17 down-regulated MYC in the context of Hepatitis B infection in human hepatocytes.

FIG. 11 shows that targeting KRAB effector (or no-effector or NE) fused to zinc-finger domain to the upstream region directly adjacent to the CTCF motif (ZF3-NE or ZF3-KRAB) and targeting MQ1 effector fused to Zinc-Finger domains to the MYC promoter (ZF9-MQ1) downregulated MYC1 mRNA expression.

FIG. 12 shows that ZF3-KRAB plus ZF9-MQ1 down-regulated MYC to a greater degree than ZF9-MQ1 alone or ZF3-NE plus ZF9-MQ1 combination.

FIG. 13A shows that ZF9-MQ1 designed to bind and target the MYC promoter was dosed at multiple concentration in five HCC cells line, Hep3B, HepG2, SKHEP1, SNU-182 and SNU-449.

FIG. 13B-F shows that ZF9-MQ1 downregulated MYC expression and reduced viability in all five HCC cell lines tested and ZF9-MQ1 downregulated MYC expression with a median EC50 of 0.028 ug/ml LNP/mRNA with a ˜10-fold higher median EC50 effect on viability (0.13 ug/ml) in vitro at 72 hours in a HepG2 cell line.

FIG. 14 shows that ZF9-MQ1 was able to significantly reduced tumor growth (from day 6 forward) when compared to PBS control treated mice and ZF9-MQ1 reduced tumor growth more than the small molecule comparator (MYCi975) (A). ZF9-MQ1 had minimal effect on overall animal weight compared to PBS or MYCi975 (B).

FIG. 15A shows that combination of ZF9-MQ1 and ZF3-KRAB at 1.5 mg/kg every 5 days for 2 doses, 3 mg/kg every 5 days for 3 doses, 3 mg/kg every 3 days for 1 dose reduced tumor growth at a comparable level to sorafenib.

FIG. 15B shows that treatment with a combination of ZF9-MQ1 and ZF3-KRAB had minimal effect on overall animal weight compared to the effect on overall animal weight when treated with sorafenib.

FIG. 16A shows that that ZF9-MQ1 (from day 13 forward) and co-formulation of ZF9-MQ1 and ZF3-KRAB (from day 6 forward) at 1 mg/kg was able to significantly reduce tumor growth when compared to negative control treated mice.

FIG. 16B shows that ZF9-MQ1 individually and the co-formulation of ZF9-MQ1 and ZF3-KRAB at 3 mg/kg was able to reduce tumor growth when compared to negative control treated mice.

FIG. 16C shows that the co-formulation of ZF9-MQ1 and ZF3-KRAB was able to reduce tumor growth at a similar or a greater level than cisplatin or the small molecule comparator (MYCi975) at both 1 mg/kg and 3 mg/kg dosage.

FIG. 16D shows that treatment with a co-formulation of ZF9-MQ1 and ZF3-KRAB at both 1 mg/kg and 3 mg/kg dosage had minimal effect on overall animal weight compared to the effect on overall animal weight when treated with either cisplatin or MYCi975.

FIG. 17A shows that ZF9-MQ1 reduced MYC mRNA level by over 80% in A549 cell line 120 hours post-treatment.

FIG. 17B shows that ZF9-MQ1 reduced MYC mRNA level by over 80% in NCI-H2009 cell line 120 hours post-treatment.

FIG. 17C shows that ZF9-MQ1 reduced MYC mRNA level by over 80% in NCI-H358 cell line 120 hours post-treatment.

FIG. 17D shows that ZF9-MQ1 reduced MYC mRNA level by over 80% in HCC95 cell line 72 hours post-treatment.

FIG. 17E shows that ZF9-MQ1 caused loss of cell viability in A549 cell line 120 hours post-treatment.

FIG. 17F shows that ZF9-MQ1 caused loss of cell viability in NCI-H2009 cell line 120 hours post-treatment.

FIG. 17G shows that ZF9-MQ1 caused loss of cell viability in NCI-H358 cell line 120 hours post-treatment.

FIG. 17H shows that ZF9-MQ1 caused loss of cell viability in HCC95 cell line 72 hours post-treatment.

FIG. 18A shows that 96 hours post-treatment, about ˜17.5% cells were apoptotic in the untreated cell population.

FIG. 18B shows that 96 hours post-treatment, about ˜18% cells were apoptotic in the cell population treated with ZF9-NE.

FIG. 18C shows that 96 hours post-treatment, about ˜38.9% cells were apoptotic in the cell population treated with ZF9-MQ1.

FIG. 18D shows that 96 hours post-treatment, about ˜38.9% cells were apoptotic in the cell population treated with ZF9-MQ1 in contrast to ˜18% apoptotic cells in both untreated cells and ZF9-NE treated cell population indicating that ZF9-MQ1 is capable of inducing cellular apoptosis of lung cancer cells.

FIGS. 19A and B show ZF9-MQ1 down-regulated MYC with an EC50 of 0.08 ug/ml LNP/mRNA with a ˜25-fold higher EC50 effect on viability (2 ug/ml) in vitro at 72 hours in these A549 (FIG. 19A) and HCC95 (FIG. 19B) cell line.

FIGS. 20A and B show that ZF9-MQ1 treatment reduces MYC protein levels over 80% at 96 hours post-treatment in lung cancer cell lines.

FIG. 21 shows that ZF9-MQ1 was able to significantly reduce tumor growth (from day 8 forward when compared to PBS control treated mice. It was also observed that ZF9-MQ1 had minimal effect on overall animal weight.

FIG. 22 shows that guide RNA GD-29833 and 29914 could effectively downregulate MYC mRNA levels when delivered with a dCAS9-KRAB effector mRNA using LNP delivery with SSOP, highlighting the ability to decrease oncogenic MYC using this distal regulatory element.

FIG. 23 shows that guide RNA GD-29833 and 29914 could effectively downregulate MYC mRNA levels when delivered with a dCAS9-KRAB effector mRNA using LNP delivery with MC3, highlighting the ability to decrease oncogenic MYC using this distal regulatory element.

FIG. 24A shows that guide RNA GD-29833 and 29914 could effectively downregulate MYC mRNA levels when delivered with all 3 effector proteins (EZH2, EZH2-KRAB, and MQ1) in A549 cell line.

FIG. 24B shows that guide RNA GD-29833 and 29914 could effectively downregulate MYC mRNA levels when delivered with all 3 effector proteins (EZH2, EZH2-KRAB, and MQ1) in NCI-H2009 cell line.

FIG. 25A shows that guide RNA GD-29833 and 29914 delivered with KRAB or MQ1 could significantly downregulate MYC mRNA levels in A549 cell line 120 hours post treatment.

FIG. 25B shows that guide RNA GD-29833 and 29914 delivered with KRAB or MQ1 could significantly downregulate MYC mRNA levels in NCI-H2009 cell line 120 hours post treatment and the downregulation is comparable to the downregulation observed after ZF9-MQ1 treatment.

FIG. 26A shows that dCas9-MQ1 increased target site methylation in NSCLC to about 60%.

FIG. 26B shows that dCas9-MQ1 directed methylation to the distal promoter region (increased to about 50%).

FIGS. 27A-B show that directing guides to the MYC lung super-enhancer with transcriptional repressors reduces MYC protein levels at 96 hours in NCI-H2009 lung cancer cell lines.

FIG. 28A shows the ZF9-MQ1 protein presence in whole cell lysate decreases gradually after treating the Hep3B cell with ZF9-MQ1.

FIG. 28B shows the MYC protein expression in whole cell lysate downregulates gradually after treating the Hep3B cell with ZF9-MQ1.

FIG. 28C shows the ZF9-MQ1 protein presence in whole cell lysate correlates with down regulation of MYC protein after treating the Hep3B cell with ZF9-MQ1.

FIG. 29A shows that down regulation of mRNA expression with a 45% down-regulation in MYC transcript at several timepoints through Day 15 in SK-HEP cell line after treatment with ZF9-MQ1.

FIG. 29B shows that MYC transcriptional changes correlated with the percentage of methylation out to day 15.

FIG. 30A shows that primary hepatocytes treated with ZF9-MQ1, ZF9-MQ1+ZF3-KRAB, or bi-cistronic ZF9-MQ1_ZF3-KRAB at concentrations 0.6 μg/ml, 1.25 μg/ml, and 2.5 μg/ml showed a decrease of MYC mRNA expression when compared to GFP, ZF-NE, or ZF3-KRAB alone.

FIG. 30B shows that treatment with ZF9-MQ1, ZF9-MQ1+ZF3-KRAB, or bi-cistronic ZF9-MQ1_ZF3-KRAB at concentrations 0.6 μg/ml, 1.25 μg/ml, and 2.5 μg/ml had minimal effect on viability of primary hepatocytes, demonstrating that the decrease in MYC expression is less consequential to normal cells when compared to HCC cell lines.

FIG. 30C shows that primary hepatocytes treated with ZF9-MQ1, ZF9-MQ1+ZF3-KRAB, or bi-cistronic ZF9-MQ1_ZF3-KRAB at concentrations 0.5 μg/ml, 1.0 μg/ml, and 2.0 μg/ml showed a decrease of MYC mRNA expression when compared to GFP, ZF-NE, or ZF3-KRAB alone.

FIG. 30D shows that treatment with ZF9-MQ1, ZF9-MQ1+ZF3-KRAB, or bi-cistronic ZF9-MQ1_ZF3-KRAB at concentrations 0.5 μg/ml, 1.0 μg/ml, and 2.0 μg/ml had minimal effect on viability of primary hepatocytes, demonstrating that the decrease in MYC expression is less consequential to normal cells when compared to HCC cell lines.

FIG. 31A shows that treatment with ZF9-MQ1+ZF3-KRAB showed a statistically significant reduction in tumor size following three administrations, resulting in a 63% lower tumor volume at Day 25 compared to control and that ZF9-MQ1+ZF3-KRAB treatment was associated with an equivalent effect on tumor volume to treatment with cisplatin.

FIG. 31B showed that mice treated with ZF9-MQ1+ZF3-KRAB did not experience a significant decrease in body weight.

FIG. 32A shows that treatment with ZF9-MQ1+ZF3-KRAB at 1.5 mg/kg was associated with a statistically significant reduction in tumor size following two administrations, resulting in 63% inhibition of tumor growth by Day 23 compared to negative control. ZF9-MQ1+ZF3-KRAB at 3 mg/kg was associated with a statistically significant reduction in tumor size following two administrations, resulting in 54% inhibition of tumor growth by Day 23 compared to negative control, and treatment with a 6 mg/kg dose of ZF9-MQ1+ZF3-KRAB is associated with a statistically significant reduction in tumor size following two administrations, resulting in 63% lower tumor volume at Day 23 compared to negative control.

FIG. 32B shows that mice treated with ZF9-MQ1+ZF3-KRAB did not experience a significant decrease in body weight. Mice treated with sorafenib experienced an initial drop in body weight with a later gain in overall body weight potentially due to an increase in tumor mass.

FIG. 33A shows that the bi-cistronic construct ZF9-MQ1_ ZF3-KRAB downregulated MYC mRNA expression at concentrations 0.6 μg/ml and 2.0 μg/ml in Hep 3B cells to a greater extent than the single constructs (ZF3-KRAB or ZF9-MQ1) alone. Bi-cistronic ZF9-MQ1_ ZF3-KRAB reduced total MYC mRNA levels by 99% at 48 hours at both 0.6 μg/ml and 2 μg/ml concentrations.

FIG. 33B shows that the bi-cistronic construct ZF9-MQ1_ ZF3-KRAB downregulated cell viability in Hep 3B cells to a greater extent than the single constructs (ZF3-KRAB or ZF9-MQ1) alone. Bi-cistronic ZF9-MQ1_ ZF3-KRAB reduced the viability of Hep3B cells by about 80% and 27% respectively at both 0.6 μg/ml and 2 μg/ml concentrations.

FIG. 34A shows that the bi-cistronic construct ZF9-MQ1_ ZF3-KRAB downregulated MYC mRNA and cell viability in Hep3B cells in a dose-dependent manner.

FIG. 34B shows that the bi-cistronic construct ZF9-MQ1_ ZF3-KRAB downregulated MYC mRNA and cell viability in HepG2 cells in a dose-dependent manner.

FIG. 34C shows that the bi-cistronic construct ZF9-MQ1_ ZF3-KRAB downregulated MYC mRNA and cell viability in SKHEP1 cells in a dose-dependent manner.

FIG. 34D shows bi-cistronic ZF9-MQ1_ZF3-KRAB bi-cistronic construct ZF9-MQ1_ ZF3-KRAB was effective against both HCC S1 and S2 subtype.

FIG. 35 shows at 48 hours of treatment with bi-cistronic ZF9-MQ1_ZF3-KRAB>75% apoptotic cells were detected in the Hep 3B and Hep G2 cell lines and about 15% apoptotic cells were detected in the SK-HEP-1 cell line. Cells were unaffected by non-coding mRNA control compared to untreated cells (5-20% background apoptosis).

FIG. 36 shows, in SKHEP1 cells, after 1 treatment with the bi-cistronic ZF9-MQ1_ZF3-KRAB construct the MYC mRNA levels were reduced at day one and remained repressed up to fifteen days following the treatment.

FIG. 37 shows, bi-cistronic ZF9-MQ1_ZF3-KRAB treatment decreased MYC mRNA and protein expression at 6 hours which remained down 96 hours later when compared to short non-coding mRNA or untreated cells in both Hep3B and SKHEP1 cell line.

FIG. 38 shows at both 6 and 24 hour timepoints following transfection, both OEC ZF3-KRAB and ZF9-MQ1 proteins encoded by bi-cistronic ZF9-MQ1_ZF3-KRAB mRNA were visualized by HA tag on a western blot.

FIG. 39A shows the IC50 of sorafenib in SKHEP1 reduced from 12.3 to 10.7 μM when sorafenib was administered in combination with 0.6 μg/ml bi-cistronic ZF9-MQ1_ZF3-KRAB. The IC₅₀ of sorafenib did not change significantly in SKHEP1 cells when sorafenib was administered in combination with 0.1 μg/ml bi-cistronic ZF9-MQ1_ZF3-KRAB.

FIG. 39B shows the IC50 of sorafenib in Hep 3B reduced from 4.4 to 2.9 μM when sorafenib, was administered in combination with 0.6 μg/ml bi-cistronic ZF9-MQ1_ZF3-KRAB. The IC₅₀ of sorafenib did not change significantly in Hep 3B cells when sorafenib was administered in combination with 0.1 μg/ml bi-cistronic ZF9-MQ1_ZF3-KRAB.

FIG. 40A shows, the IC₅₀ of JQ1 in SKHEP1 cells reduced when treated with bi-cistronic ZF9-MQ1_ZF3-KRAB at concentrations 0.6 μg/ml and 0.1 μg/ml respectively.

FIG. 40B shows the IC₅₀ of JQ1 in Hep 3B cells reduced when treated with bi-cistronic ZF9-MQ1_ZF3-KRAB at concentrations 0.6 μg/ml and 0.1 μg/ml respectively.

FIG. 41A shows ZF17-MQ1 was able to downregulate mouse MYC mRNA expression in Hepa1-6 cells compared to untreated cells at both 0.6 and 1.2 μg/ml concentrations.

FIG. 41B shows ZF17-MQ1 was able to reduce cell viability in mouse Hepa1-6 cells compared to untreated cells at both 0.6 and 1.2 μg/ml concentrations.

FIG. 42A shows ZF17-MQ1 treatment in mouse HCC cells Hepa1-6 showed significant downregulation of MYC protein at 24 and 48 hours.

FIG. 42B shows ZF17-MQ1 treatment in mouse HCC cells Hepa1-6 showed significant downregulation of MYC protein at 24 and 48 hours.

FIG. 42C shows ZF17-MQ1 treatment in mouse HCC cells Hepa1-6 showed significant downregulation of MYC mRNA at 96 hours.

FIG. 42D shows ZF17-MQ1 treatment in mouse HCC cells Hepa1-6 showed significant loss of cell viability at 96 hours.

FIG. 43 shows ZF17-MQ1 significantly reduced animal tumor burden after 4 doses and following a drug holiday of two weeks, re-treatment of the mice with ZF17-MQ1 resulted in full tumor depletion after ˜4 weeks.

FIG. 44A shows ZF17-MQ1 treated cells showed reduced MYC protein levels in LL2 cells in comparison to untreated or GFP-treated cells.

FIG. 44B shows compared to levels observed in untreated cells, ZF17-MQ1 and ZF16-MQ1 reduced MYC mRNA levels by >99.9% or 74%, respectively in LL2 cells.

FIG. 44C shows all three constructs, ZF15-MQ1, ZF16-MQ1, and ZF17-MQ1 were able to reduce cell viability in LL2 cell to a greater extent than untreated and GFP-treated cells.

FIG. 45A shows ZF17-MQ1 reduced MYC mRNA level at both 1.25 μg/mL and 2.5 μg/mL concentrations. Compared to levels observed in untreated cells, at 2.5 μg/mL ZF17-MQ1 reduced MYC mRNA levels by 93% and 85% in LL2 and CT26 cells, respectively.

FIG. 45B shows ZF17-MQ1 reduced cell viability at both concentrations. Compared to untreated cells, under these conditions, ZF17-MQ1 reduced cell viability by 87% and 93% in LL2 and CT26 cells, respectively.

FIG. 46 shows ZF17-MQ1 downregulated MYC mRNA and reduces cell viability in CMT167 and LL2 cells to a greater extent than untreated and GFP-treated cells (negative control). Compared to levels observed in untreated cells, ZF17-MQ1 reduced MYC mRNA levels by 62% and 73% in CMT167 and LL2 cells, respectively. Furthermore, compared to untreated cells, under these conditions, ZF17-MQ1 reduced cell viability by 54% and 57% in CMT167 and LL2 cells, respectively.

FIG. 47 shows ZF9-MQ1 downregulated MYC mRNA levels by 94%, 96%, 96% levels compared to untreated cells in primary small airway epithelial cells, primary lobar epithelial cells, and primary lung fibroblasts respectively. However, viability was only reduced by 16%, 9%, and 22% compared to control cells.

FIG. 48A shows ZF9-MQ1 and JQ1 each separately inhibited cell viability of A549 cells.

FIG. 48B shows ZF9-MQ1 (0.5 μg/ml) and JQ1 (concentrations up to 6.25 uM) showed a greater than additive effect on the inhibition of A549 viability than what was predicted by their individual activities.

FIG. 48C shows ZF9-MQ1 (1.0 μg/ml) and JQ1 (concentrations up to 6.25 uM) showed a greater than additive effect on the inhibition of A549 viability than what was predicted by their individual activities.

FIG. 49A shows ZF9-MQ1 and BET762 each separately inhibited cell viability of A549 cells.

FIG. 49B shows ZF9-MQ1 (0.5 μg/ml) and BET762 (concentrations up to 1.25 uM) showed a greater than additive effect on the inhibition of A549 viability than what was predicted by their individual activities.

FIG. 49C shows ZF9-MQ1 (1.0 μg/ml) and BET762 (concentrations up to 0.625 uM) showed a greater than additive effect on the inhibition of A549 viability than what was predicted by their individual activities.

FIG. 50A shows ZF9-MQ1 and Birabresib each separately inhibited cell viability of A549 cells.

FIG. 50B shows ZF9-MQ1 (0.5 μg/ml) and Birabresib (concentrations up to 0.625 uM) showed a greater than additive effect on the inhibition of A549 viability than what was predicted by their individual activities.

FIG. 50C shows ZF9-MQ1 (1.0 μg/ml) and Birabresib (concentrations up to 0.313 uM) showed a greater than additive effect on the inhibition of A549 viability than what was predicted by their individual activities.

FIG. 51A shows ZF9-MQ1 and Trametinib each separately inhibited cell viability of A549 cells.

FIG. 51B shows ZF9-MQ1 (0.5 μg/ml) and Trametinib (concentrations up to 0.05 uM) showed a greater than additive effect on the inhibition of A549 viability than what was predicted by their individual activities.

FIG. 51C shows ZF9-MQ1 (1.0 μg/ml) and Trametinib (concentrations up to 0.05 uM) showed a greater than additive effect on the inhibition of A549 viability than what was predicted by their individual activities.

FIG. 52A shows all the constructs ZF9-MQ1, ZF54-KRAB, ZF67-KRAB, and ZF68-KRAB were able to downregulate MYC mRNA levels in H2009 cells by at least 42% at 72 hours post-treatment compared to untreated cells.

FIG. 52B shows the constructs ZF9-MQ1, ZF67-KRAB, and ZF68-KRAB were able to downregulate MYC mRNA levels in H226 cells by at least 27% at 72 hours post-treatment compared to untreated cells.

FIG. 52C shows both the constructs ZF9-MQ1 and ZF54-KRAB were able to downregulate MYC mRNA levels in H226 cells by at least 27% at 72 hours post-treatment compared to untreated cells.

FIG. 52D shows the constructs ZF9-MQ1, ZF61-KRAB, ZF67-KRAB, and ZF68-KRAB were able to downregulate MYC mRNA levels in H460 cells by at least 26% at 72 hours post-treatment compared to untreated cells.

FIG. 53 shows at the highest concentration tested, ZF9-MQ1 and ZF54-KRAB each separately downregulated MYC mRNA in H2009 cells by 99% or 62% respectively, relative to untreated control cells. When less than 0.313 μg/mL ZF9-MQ1 is combined with 1 or 2 μg/mL ZF54-KRAB, MYC mRNA is downregulated to a greater extent than that observed for either treatment alone.

FIG. 54 shows ZF9-MQ1 downregulated MYC mRNA in H1299 cells by 95% relative to untreated control cells by 48 hours and maintained downregulation at 90% of control levels at 144 hours. Combination of ZF9-MQ1 plus ZF54-KRAB reduced MYC mRNA levels to 98% at 48 hours and maintained downregulation to 93% of control levels at 144 hours (FIG. 54). Further, the data showed ZF9-MQ1 and ZF9-MQ1 combined with ZF54-KRAB downregulated MYC mRNA levels in H1299 cells for at least 6 days.

FIG. 55 shows 24 hours after introduction to H2009 cells, ZF9-MQ1 and ZF54-KRAB downregulated MYC mRNA levels by up to 83% and 55%, respectively, in comparison to untreated cells. MYC mRNA levels were further reduced by another 13% in ZF9-MQ1-treated cells to 96% of untreated controls 48 hours after treatment, whereas ZF54-KRAB does not further downregulate MYC levels. MYC mRNA levels in cells treated with ZF9-MQ1_ZF54-KRAB and ZF54-KRAB_ZF9-MQ1 were reduced to 95% and 96% of control cells, respectively, at 24 hours post-treatment. The data indicated that these controllers were able to reduce MYC mRNA levels earlier than ZF9-MQ1 leading to a greater level of MYC downregulation in treated cells compared ZF9-MQ1 treated cells at 24 hours in H2009 cells.

FIG. 56 shows ZF9-MQ1 treatment inhibited tumor growth in the H460 subcutaneous tumor model at a similar or higher level compared to sorafenib induced tumor growth inhibition.

### DETAILED DESCRIPTION OF CERTAIN EMBODIMENTS

The present disclosure provides technologies for modulating, e.g., decreasing, expression of a target gene e.g., MYC in cell, e.g., in a subject or patient, through the use of an expression repressor or a system described herein.

Many different diseases and syndromes, including cancer, autoimmunity, cardiovascular disease, and obesity, can be caused by mis-regulation of gene expression. Particularly, overexpression of transcription factors has long been known to known to contribute to tumorigenesis, and recent studies indicate that overexpressed oncogenic transcription factors can alter the core autoregulatory circuitry of the cell.

MYC, a transcription factor and master cell regulator, is frequently dysregulated in over 50% of human cancer and plays a central role in nearly every aspect of the tumorigenic process. Except for early response genes, MYC typically upregulates gene expression. MYC is the most frequently amplified oncogene, and the elevated expression of its gene product is associated with tumor aggression and poor clinical outcome. Elevated levels of c-MYC can promote tumorigenesis in a wide range of tissues. Most tumor cells depend on the transcription factor c-MYC for their growth and proliferation. MYC overexpression is also associated in chronic liver disease e.g., viral and alcohol related liver disease. MYC overexpression level varies in specific cancer subtypes. Without wishing to be bound by theory, it is thought that modulating e.g., decreasing the levels of MYC in a subject (e.g., overall, or in a specific target tissue or tissues) suffering from MYC mis-regulation disorder may lessen or eliminate the symptoms of the MYC mis-regulation disorder.

The present disclosure provides, in part, an expression repressor comprising a targeting moiety that binds to a target gene promoter, e.g., MYC promoter or operably linked to the target gene, e.g., MYC gene and an effector moiety capable of modulating (e.g., decreasing) expression of the target gene, e.g., MYC when localized by the targeting moiety. In some embodiments, the expression repressors disclosed herein specifically bind to an expression control element (e.g., a promoter or enhancer, repressor or silencer) operably linked to the target gene, e.g., MYC via the targeting moiety and the effector moiety modulates expression of the target gene, e.g., MYC. In some embodiments, the expression repressors disclosed herein specifically bind to an anchor sequence of an anchor sequence mediated conjunction (ASMC) comprising a target gene, e.g., MYC or to a sequence proximal to the anchor sequence via the targeting moiety and the effector moiety modulates expression of the target gene, e.g., MYC. In some embodiments, the expression repressors disclosed herein specifically bind to a genomic locus located in a super enhancer region of a target gene, e.g., MYC and the effector moiety modulates expression of the target gene, e.g., MYC.

The disclosure further provides in part, an expression repression system comprising two or more expression repressors, each comprising a targeting moiety and optionally an effector moiety. In some embodiments, the targeting moieties target two or more different sequences (e.g., each expression repressor may target a different sequence). In some embodiments, the first expression repressor binds to a transcription regulatory element (e.g., a promoter or transcription start site (TSS)) operably linked to a target gene, e.g., MYC and the second expression repressor binds to an anchor sequence of an anchor sequence mediated conjunction (ASMC) comprising a target gene, e.g., MYC. In some embodiments, the first expression repressor binds to a transcription regulatory element (e.g., a promoter or transcription start site (TSS)) operably linked to a target gene, e.g., MYC and the second expression repressor binds to an expression control element (e.g., an enhancer, a super-enhancer, a repressor, or a silencer) operably linked to a target gene, e.g., MYC. In some embodiments, the first expression repressor binds to an anchor sequence of an anchor sequence mediated conjunction (ASMC) comprising a target gene, e.g., MYC and the second expression repressor binds to an expression control element (e.g., an enhancer, a super-enhancer, a repressor, or a silencer) operably linked to a target gene. Generally, modulation of expression of a target gene, e.g., MYC by an expression repression system involves the binding of the first expression repressor and second expression repressor to the first and second DNA sequences, respectively. Binding of the first and second DNA sequences localizes the functionalities of the first and second effector moieties to those sites. Without wishing to be bound by theory, in some embodiments employing the functionalities of both the first and second expressor moieties stably represses expression of a target gene associated with or comprising the first and/or second DNA sequences, e.g., wherein the first and/or second DNA sequences are or comprise sequences of the target gene or one or more operably linked transcription control elements. In some embodiments, the expression repressor system is encoded by a bi-cistronic nucleic acid sequence.

The disclosure further provides nucleic acids encoding said expression repressors and/or expression repressor systems, compositions comprising expression repressors and/or expression repressor systems, and methods for delivering said nucleic acids. Further provided are methods for increasing target gene expression, e.g., MYC gene expression in a cell using the expression repressors or expression repressor systems described herein.

Expression Repressors

As described herein, the present disclosure in part provides expression repressors for modulating, e.g., decreasing the expression of a target gene, e.g., MYC. In some embodiments, an expression repressor may comprise a targeting moiety that binds to a target gene promoter, e.g., MYC promoter and optionally an effector moiety. In some embodiments, the targeting moiety specifically binds a target DNA sequence, e.g., MYC DNA sequence, thereby localizing the expression repressor's functionality to the DNA sequence. In some embodiments, an expression repressor comprises a targeting moiety and one effector moiety. In some embodiments an expression repressor comprises a targeting moiety and a plurality of effector moieties (e.g., 1, 2, 3, 4, 5, 6, 7, 8, 9, 10, 11, 12, 13, 14, 15, 16, 17, 18, 19, 20, or more effector domains (and optionally, less than 20, 19, 18, 17, 16, 15, 14, 13, 12, 11, 10, 9, 8, 7, 6, 5, 4, 3, or 2 effector domains)).

An expression repressor may comprise a plurality of effector moieties, where each effector moiety comprises a different functionality than the other effector moieties. For example, an expression repressor may comprise two effector moieties, where the first effector moiety comprises DNA methylase functionality and the second effector moiety comprises a transcriptional repressor functionality. In some embodiments, an expression repressor comprises effector moieties whose functionalities are complementary to one another with regard to decreasing expression of a target gene, e.g., MYC, where the functionalities together enable inhibition of expression and, optionally, do not inhibit or negligibly inhibit expression when present individually. In some embodiments, an expression repressor comprises a plurality of effector moieties, wherein each effector moiety complements each other effector moiety, each effector moiety decreases expression of a target gene, e.g., MYC.

In some embodiments, an expression repressor comprises a combination of effector moieties whose functionalities synergize with one another with regard to decreasing expression of a target gene, e.g., MYC. Without wishing to be bound by theory, in some embodiments, epigenetic modifications to a genomic locus are cumulative, in that multiple transcription activating epigenetic markers (e.g., multiple different types of epigenetic markers and/or more extensive marking of a given type) individually together inhibit expression more effectively than individual modifications alone (e.g., producing a greater decrease in expression and/or a longer-lasting decrease in expression). In some embodiments, an expression repressor comprises a plurality of effector moieties, wherein each effector moiety synergizes with each other effector moiety, e.g., each effector moiety decreases expression of a target gene, e.g., MYC. In some embodiments, an expression repressor (comprising a plurality of effector moieties which synergize with one another) is more effective at inhibiting expression of a target gene, e.g., MYC than an expression repressor comprising an individual effector moiety. In some embodiments, an expression repressor comprising said plurality of effector moieties is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at decreasing expression of a target gene, e.g., MYC than an expression repressor comprising an individual effector moiety.

In some embodiments, an expression repressor comprises one or more targeting moieties e.g., a Cas domain, TAL effector domain, or Zn Finger domain. In an embodiment, when an expression repressor system comprises two or more targeting moieties of the same type, e.g., two or more Cas domains, the targeting moieties specifically bind two or more different sequences. For example, in an expression repressor system comprising two or more Cas domains, the two or more Cas domains may be chosen or altered such that they only appreciably bind the gRNA corresponding to their target sequence (e.g., and do not appreciably bind the gRNA corresponding to the target of another Cas domain).

In some embodiments, an expression repressor comprises a targeting moiety and an effector moiety that are covalently linked, e.g., by a peptide bond. In some embodiments, the targeting moiety and the effector moiety are situated on the same polypeptide chain, e.g., connected by one or more peptide bonds and/or a linker. In some embodiments, the expression repressor is or comprises a fusion molecule, e.g., comprising the targeting moiety and the effector moiety linked by a peptide bond and/or a linker. In some embodiments, the expression repressor comprises a targeting moiety that is disposed N-terminal of an effector moiety on the same polypeptide chain. In some embodiments, the expression repressor comprises a targeting moiety that is disposed C-terminal of an effector moiety on the same polypeptide chain. In some embodiments, an expression repressor comprises a targeting moiety and an effector moiety that are covalently linked by a non-peptide bond. In some embodiments, a targeting moiety is conjugated to an effector moiety by a non-peptide bond. In some embodiments, an expression repressor comprises a targeting moiety and a plurality of effector moieties, wherein the targeting moiety and the plurality of effector moieties are covalently linked, e.g., by peptide bonds (e.g., the targeting moiety and plurality of effector moieties are all connected by a series of covalent bonds, although each individual moiety may not share a covalent bond with every other effector moiety).

In other embodiments, an expression repressor comprises a targeting moiety and an effector moiety that are not covalently linked, e.g., that are non-covalently associated with one another. In some embodiments, an expression repressor comprises a targeting moiety that non-covalently binds to an effector moiety or vice versa. In some embodiments, an expression repressor comprises a targeting moiety and a plurality of effector moieties, wherein the targeting moiety and at least one effector moiety are not covalently linked, e.g., are non-covalently associated with one another, and wherein the targeting moiety and at least one other effector moiety are covalently linked, e.g., by a peptide bond.

In general, an expression repressor as described herein binds (e.g., via a targeting moiety) a genomic sequence element proximal to and/or operably linked to a target gene, e.g., MYC. In some embodiments, binding of the expression repressor to the genomic sequence element modulates (e.g., decreases) expression of the target gene, e.g., MYC. For example, binding of an expression repressor comprising an effector moiety that recruits or inhibits recruitment of components of the transcription machinery to the genomic sequence element may modulate (e.g., decrease) expression of the target gene, e.g., MYC. As a further example, binding of an expression repressor comprising an effector moiety with an enzymatic activity (e.g., an epigenetic modifying moiety) may modulate (e.g., decrease) expression of the target gene, e.g., MYC) through the localized enzymatic activity of the effector moiety. As a further example, both binding of an expression repressor to a genomic sequence element and the localized enzymatic activity of an expression repressor may contribute to the resulting modulation (e.g., decrease) in expression of the target gene, e.g., MYC.

In some embodiments, an expression repressor comprises an effector moiety wherein the effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB (e.g., a KRAB domain), MeCP2, HP1, RBBP4, REST, FOG1, SUZ12 or a functional variant or fragment thereof.

In some embodiments, an expression repressor comprises a first effector moiety and a second effector moiety, wherein the first effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB (e.g., a KRAB domain), MeCP2, HP1, RBBP4, REST, FOG1, SUZ12 or a functional variant or fragment thereof, and the second effector moiety comprises a different protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB (e.g., a KRAB domain), MeCP2, HP1, RBBP4, REST, FOG1, SUZ12, or a functional variant or fragment thereof.

In some embodiments, the disclosure provides nucleic acid sequences encoding an expression repressor, an expression repressor system, a targeting moiety and/or an effector moiety as described herein. A skilled artisan is aware that the nucleic acid sequences of RNA are identical to the corresponding DNA sequences, except that typically thymine (T) is replaced by uracil (U). It will be understood that when a nucleotide sequence is represented by a DNA sequence (e.g., comprising, A, T, G, C), this disclosure also provides the corresponding RNA sequence (e.g., comprising, A, U, G, C) in which “U” replaces “T.” Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

It will be appreciated by those skilled in the art that due to the degeneracy of the genetic code, a multitude of nucleotide sequences encoding an expression repressor comprising targeting moiety and/or an effector moiety as described herein may be produced, some of which have similarity, e.g., 90%, 95%, 96%, 97%, 98%, or 99% identity to the nucleic acid sequences disclosed herein. For instance, codons AGA, AGG, CGA, CGC, CGG, and CGU all encode the amino acid arginine. Thus, at every position in the nucleic acid molecules of the invention where an arginine is specified by a codon, the codon can be altered to any of the corresponding codons described above without altering the encoded polypeptide.

In some embodiments a nucleic acid cohesion encoding an expression repressor comprising a targeting moiety and/or an effector moiety may be part or all of a codon-optimized coding region, optimized according to codon usage in mammals, e.g., humans. In some embodiments, a nucleic acid cohesion encoding a targeting moiety and/or an effector moiety is codon optimized for increasing the protein expression and/or increasing the duration of protein expression. In some embodiments, a protein produced by the codon optimized nucleic acid sequence is at least 1%, at least 2%, at least 5%, at least 10%, at least 15%, at least 20%, at least 30%, at least 40%, or at least 50% higher compared to levels of the protein when encoded by a nucleic acid sequence that is not codon optimized.

### Expression Repression Systems

Expression repression systems of the present disclosure may comprise two or more expression repressors. In some embodiments, an expression repression system comprises 2, 3, 4, 5, 6, 7, 8, 9, 10, 11, 12, or more expression repressors (and optionally no more than 15, 14, 13, 12, 11, 10, 9, 8, 7, 6, 5, 4, 3, or 2). In some embodiments, an expression repression system targets two or more different sequences (e.g., a 1^{st} and 2^{nd}, 3^{rd}, 4^{th}, 5^{th}, 6^{th}, 7^{th}, 8^{th}, 9^{th}, 10^{th}, 11^{th}, 12^{th}, and/or further DNA sequence, and optionally no more than a 20^{th}, 19^{th}, 18^{th}, 17^{th}, 16^{th}, 15^{th}, 14^{th}, 13^{th}, 12^{th}, 11^{th}, 10^{th}, 9^{th}, 8^{th}, 6^{th}, 5^{th}, 4^{th}, 3^{rd}, or 2^{nd} sequence). In some embodiments, an expression repression system comprises a plurality of expression repressors, wherein each member of the plurality of expression repressors does not detectably bind, e.g., does not bind, to another member of the plurality of expression repressors. In some embodiments, an expression repression system comprises a first expression repressor and a second expression repressor, wherein the first expression repressor does not detectably bind, e.g., does not bind, to the second expression repressor.

In some embodiments, an expression repression system of the present disclosure comprises two or more expression repressors, wherein the expression repressors are present together in a composition, pharmaceutical composition, or mixture. In some embodiments, an expression repression system of the present disclosure comprises two or more expression repressors, wherein one or more expression repressors is not admixed with at least one other expression repressor. For example, an expression repression system may comprise a first expression repressor and a second expression repressor, wherein the presence of the first expression repressor in the nucleus of a cell does not overlap with the presence of the second expression repressor in the nucleus of the same cell, wherein the expression repression system achieves a decrease in expression of a MYC gene via the non-overlapping presences of the first and second expression repressors. In some embodiments, the expression repression system achieves a greater decrease in expression of a MYC gene in comparison to the decrease in expression of a MYC gene achieved by the first or the second expression repressor alone.

In some embodiments, the expression repressors of an expression repressor system each comprise a different targeting moiety (e.g., the first, second, third, or further expression repressors each comprise different targeting moieties from one another). For example, an expression repression system may comprise a first expression repressor and a second expression repressor wherein the first expression repressor comprises a first targeting moiety (e.g., a Cas9 domain, TAL effector domain, or Zn Finger domain), and the second expression repressor comprises a second targeting moiety (e.g., a Cas9 domain, TAL effector domain, or Zn Finger domain) different from the first targeting moiety. In some embodiments, different can mean comprising distinct types of targeting moiety, e.g., the first targeting moiety comprises a Cas9 domain, and the second DNA-targeting moiety comprises a Zn finger domain. In other embodiments, different can mean comprising distinct variants of the same type of targeting moiety, e.g., the first targeting moiety comprises a first Cas9 domain (e.g., from a first species) and the second targeting moiety comprises a second Cas9 domain (e.g., from a second species). In an embodiment, when an expression repressor system comprises two or mule targeting moieties of the same type, e.g., two or more Cas9 or ZF domains, the targeting moieties specifically bind two or more different sequences. For example, in an expression repressor system comprising two or more Cas9 domains, the two or more Cas9 domains may be chosen or altered such that they only appreciably bind the gRNA corresponding to their target sequence (e.g., and do not appreciably bind the gRNA corresponding to the target of another Cas9 domain). In a further example, in an expression repressor system comprising two or more effector moieties, the two or more effector moieties may be chosen or altered such that they only appreciably bind to their target sequence (e.g., and do not appreciably bind the target sequence of another effector moiety).

In some embodiments, an expression repressor system comprises three or more expression repressors and two or more expression repressors comprise the same targeting moiety. For example, an expression repressor system may comprise three expression repressors, wherein the first and second expression repressors both comprise a first targeting moiety and the third expression repressor comprises a second different targeting moiety. For a further example, an expression repressor system may comprise four expression repressors, wherein the first and second expression repressors both comprise a first targeting moiety and the third and fourth expression repressors comprises a second different targeting moiety. For a further example, an expression repressor system may comprise five expression repressors, wherein the first and second expression repressors both comprise a first targeting moiety, the third and fourth expression repressors both comprise a second different targeting moiety, and the fifth expression repressor comprises a third different targeting moiety. As described above, different can mean comprising different types of -targeting moieties or comprising distinct variants of the same type of targeting moiety.

In some embodiments, the expression repressors of an expression repressor system each bind to a different DNA sequence (e.g., the first, second, third, or further expression repressors each bind DNA sequences that are different from one another). For example, an expression repression system may comprise a first expression repressor and a second expression repressor wherein the first expression repressor binds to a first DNA sequence, and the second expression repressor binds to a second DNA sequence. In some embodiments, different can mean that: there is at least one position that is not identical between the DNA sequence bound by one expression repressor and the DNA sequence bound by another expression repressor, or that there is at least one position present in the DNA sequence bound by one expression repressor that is not present in the DNA sequence bound by another expression repressor.

In some embodiments, the first DNA sequence may be situated on a first genomic DNA strand and the second DNA sequence may be situated on a second genomic DNA strand. In some embodiments, the first DNA sequence may be situated on the same genomic DNA strand as the second DNA sequence.

In some embodiments, an expression repressor system comprises three or more expression repressors and two or more expression repressors bind the same DNA sequence. For example, an expression repressor system may comprise three expression repressors, wherein the first and second expression repressors both bind a first DNA sequence, and the third expression repressor binds a second different DNA sequence. For a further example, an expression repressor system may comprise four expression repressors, wherein the first and second expression repressors both bind a first DNA sequence and the third and fourth expression repressors both bind a second DNA sequence. For a further example, an expression repressor system may comprise five expression repressors, wherein the first and second expression repressors both bind a first DNA sequence, the third and fourth expression repressors both bind a second DNA sequence, and the fifth expression repressor binds a third DNA sequence. As described above, different can mean that there is at least one position that is not identical between the DNA sequence bound by one expression repressor and the DNA sequence bound by another expression repressor, or that there is at least one position present in the DNA sequence bound by one expression repressor that is not present in the DNA sequence bound by another expression repressor.

In some embodiments, an expression repression system comprises two or more (e.g., two) expression repressors and a plurality (e.g., two) of the expression repressors comprise targeting moieties that bind to different DNA sequences. In such embodiments, a first targeting moiety may bind to a first DNA sequence and a second DNA-targeting moiety may bind to a second DNA sequence, wherein the first and the second DNA sequences are different and do not overlap. In some such embodiments, the first DNA sequence is separated from the second DNA sequence by at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no more than 500, 400, 300, 200, 100, 95, 90, 85, 80, 75, 70, 65, 60, 55, or 50 base pairs). In some such embodiments, the first DNA sequence is separated from the second DNA sequence by no more than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no base pairs, e.g., the first and second sequence are directly adjacent one another).

In some embodiments, the expression repressors of an expression repressor system each comprise a different effector moiety (e.g., the first, second, third, or further expression repressors each comprise a different effector moiety from one another). For example, an expression repression system may comprise a first expression repressor and a second expression repressor wherein the first expression repressor comprises a first effector moiety (e.g., comprising a DNA methyltransferase or functional fragment thereof), and the second expression repressor comprises a second effector moiety (e.g., comprising a transcription repressor (e.g., KRAB) or functional fragment thereof) different from the first effector moiety. In some embodiments, different can mean comprising distinct types of effector moiety. In other embodiments, different can mean comprising distinct variants of the same type of effector moiety, e.g., the first effector moiety comprises a first DNA methyltransferase (e.g., having a first site specificity or amino acid sequence) and the second effector moiety comprises a second DNA methyltransferase (e.g., having a second site specificity or amino acid sequence).

In some embodiments, an expression repressor system comprises a first expression repressor comprising a first effector moiety and a second expression repressor comprising a second effector moiety, wherein the first effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB, MeCP2, HP1, RBBP4, REST, FOG1, SUZ12 or a functional variant or fragment thereof, and the second effector moiety comprises a different protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB, MeCP2, HP1, RBBP4, REST, FOG1, SUZ12, or a functional variant or fragment thereof.

In some embodiments, the first or second effector moiety comprises a DNA methyltransferase activity (e.g., MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof, and the other effector moiety comprises a transcription repressor activity (e.g., KRAB, MeCP2, HP1, RBBP4, REST, FOG1, SUZ12, or a functional variant or fragment of any thereof), the first or second effector moiety comprises a histone methyltransferase activity and the other effector moiety comprises a histone deacetylase activity (e.g., HDAC1, HDAC2, HDAC3, HDAC4, MACS, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof). In some embodiments, the first or second effector moiety comprises a histone methyltransferase activity and the other effector moiety comprises a DNA methyltransferase activity (e.g., MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof). In some embodiments, the first or second effector moiety comprises a DNA methyltransferase activity and the other effector moiety comprises a transcription repressor activity. In some embodiments the first or second effector moiety comprises a histone methyltransferase activity and the other effector moiety comprises a transcription repressor activity (e.g., KRAB, MeCP2, HP1, RBBP4, REST, FOG1, SUZ12, or a functional variant or fragment of any thereof). In some embodiments, the first or second effector moiety comprises a transcription repressor activity and the other effector moiety comprises a different transcription repressor activity. In some embodiments, the first or second effector moiety comprises a DNA methyltransferase activity and the other effector moiety comprises the same DNA methyltransferase activity. In some embodiments, the first or second effector moiety comprises a DNA methyltransferase activity and the other effector moiety comprises a histone deacetylase activity. In some embodiments, the first or second effector moiety comprises a histone demethylase activity and the other effector moiety comprises a DNA methyltransferase activity. In some embodiments, the first or second effector moiety comprises a histone methyltransferase activity and the other effector moiety comprises a DNA demethylase activity. In some embodiments, the first or second effector moiety comprises a histone demethylase activity and the other effector moiety comprises a transcription repressor activity. In some embodiments, the first or second effector moiety comprises a histone demethylase activity and the other effector moiety comprises a different histone demethylase activity. In some embodiments, the first or second effector moiety comprises a histone demethylase activity and the other effector moiety comprises the same histone demethylase activity. In some embodiments, the first or second effector moiety comprises a histone deacetylase activity and the other effector moiety comprises a DNA methyltransferase activity. In some embodiments, the first or second effector moiety comprises a histone deacetylase activity and the other effector moiety comprises a DNA demethylase activity. In some embodiments, the first or second effector moiety comprises a histone deacetylase activity and the other effector moiety comprises a transcription repressor activity. In some embodiments, the first or second effector moiety comprises a histone deacetylase activity and the other effector moiety comprises a different histone deacetylase activity. In some embodiments, the first or second effector moiety comprises a histone deacetylase activity and the other effector moiety comprises the same histone deacetylase activity. In some embodiments, the first or second effector moiety comprises a DNA methyltransferase activity and the other effector moiety comprises a DNA demethylase activity. In some embodiments, the first or second effector moiety comprises a DNA demethylase activity and the other effector moiety comprises a transcription repressor activity. In some embodiments, the first or second effector moiety comprises a DNA methyltransferase activity and the other effector moiety comprises a different DNA methyltransferase activity. In some embodiments, the first or second effector moiety comprises a DNA methyltransferase activity and the other effector moiety comprises the same DNA methyltransferase activity. In some embodiments, the first or second effector moiety comprises a DNA demethylase activity and the other effector moiety comprises a transcription repressor activity. In some embodiments, the first or second effector moiety comprises a DNA demethylase activity and the other effector moiety comprises a different DNA demethylase activity. In some embodiments, the first or second effector moiety comprises a DNA demethylase activity and the other effector moiety comprises the same DNA demethylase activity. In some embodiments, the first or second effector moiety comprises a transcription repressor activity and the other effector moiety comprises a different transcription repressor activity. In some embodiments, the first or second effector moiety comprises a transcription repressor activity and the other effector moiety comprises the same transcription repressor activity.

In some embodiments, an expression repressor system comprises three or more expression repressors and two or more expression repressors comprise the same DNA-targeting moiety. For example, an expression repressor system may comprise three expression repressors, wherein the first and second expression repressors both comprise a first effector moiety and the third expression repressor comprises a second different effector moiety. For a further example, an expression repressor system may comprise four expression repressors, wherein the first and second expression repressors both comprise a first effector moiety and the third and fourth expression repressors comprises a second different effector moiety. For a further example, an expression repressor system may comprise five expression repressors, wherein the first and second expression repressors both comprise a first effector moiety, the third and fourth expression repressors both comprise a second different effector moiety, and the fifth expression repressor comprises a third different effector moiety. As described above, different can mean comprising different types of effector moiety or comprising distinct variants of the same type of effector moiety.

In some embodiments, two or more (e.g., all) expression repressors of an expression repressor system are not covalently associated with each other, e.g., each expression repressor is not covalently associated with any other expression repressor. In another embodiment, two or more expression repressors of an expression repressor system are covalently associated with one another. In an embodiment, an expression repression system comprises a first expression repressor and a second expression repressor disposed on the same polypeptide, e.g., as a fusion molecule, e.g., connected by a peptide bond and optionally a linker. In some embodiments, the peptide is a self-cleaving peptide, e.g., a T2A self-cleaving peptide. In an embodiment, an expression repression system comprises a first expression repressor and a second expression repressor that are connected by a non-peptide bond, e.g., are conjugated to one another.

### Linkers

An expression repressor or an expression repressor system as disclosed herein may comprise one or more linkers. A linker may connect a targeting moiety to an effector moiety, an effector moiety to another effector moiety, or a targeting moiety to another targeting moiety. A linker may be a chemical bond, e.g., one or more covalent bonds or non-covalent bonds. In some embodiments, a linker is covalent. In some embodiments, a linker is non-covalent. In some embodiments, a linker is a peptide linker. Such a linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length). In some embodiments, a linker can be used to space a first domain or moiety from a second domain or moiety, e.g., a DNA-targeting moiety from an effector moiety. In some embodiments, for example, a linker can be positioned between a DNA-targeting moiety and an effector moiety, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g., polypeptide) from a modulating agent, but sufficiently stable to prevent premature cleavage.

In some embodiments, one or more moieties and/or domains of an expression repressor described herein are linked with one or more linkers. In some embodiments, an expression repression may comprise a linker situated between the targeting moiety and the effector moiety. In some embodiments, the linker may have a sequence of ASGSGGGSGGARD (SEQ ID NO: 137), or ASGSGGGSGG (SEQ ID NO: 138). In some embodiments, a system comprising a first and second repressor may comprise a first linker situated between the first targeting moiety and the first effector moiety, and a second linker situated between the second targeting moiety and the second effector moiety. In some embodiments, the first and the second linker may be identical. In some embodiments, the first and the second linker may be different. In some embodiments, the first linker may comprise an amino acid sequence according to SEQ ID NO: 137 or a sequence with at least 80, 85, 90, 95, 99, or 100% identity thereto and the second linker may comprise an amino acid sequence according to SEQ ID NO: 138 or a sequence with at least 80, 85, 90, 95, 99, or 100% identity thereto.

As will be known by one of skill in the art, commonly used flexible linkers have sequences consisting primarily of stretches of Gly and Ser residues (“GS” linker). Flexible linkers may be useful for joining domains/moieties that require a certain degree of movement or interaction and may include small, non-polar (e.g., Gly) or polar (e.g., Ser or Thr) amino acids. Incorporation of Ser or Thr can also maintain the stability of a linker in aqueous solutions by forming hydrogen bonds with water molecules, and therefore reduce unfavorable interactions between a linker and moieties/domains. In some embodiments, the linker is a GS linker or a variant thereof e.g., G4S (SEQ ID NO: 207).

Rigid linkers are useful to keep a fixed distance between domains/moieties and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments. In some embodiment, the cleavable linker may be a self-cleaving linker, e.g., a T2A peptide linker. In some embodiments, the linker may comprise a “ribosome skipping” sequence, e.g., a tPT2A linker.

Examples of molecules suitable for use in linkers described herein include a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof; noncarbon linkers; carbohydrate linkers; phosphodiester linkers, or other molecule capable of covalently linking two or more components of an expression repressor. Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine, or other hydrophobic residues. Components of an expression repressor may be linked using charge-based chemistry, such that a positively charged component of an expression repressor is linked to a negative charge of another component.

Targeting Moieties

The present disclosure provides, e.g., expression repressors comprising a targeting moiety that specifically targets, e.g., binds, a genomic sequence element (e.g., a promoter, a TSS, or an anchor sequence) in, proximal to, and/or operably linked to a target gene. Targeting moieties may specifically bind a DNA sequence, e.g., a DNA sequence associated with a target gene, e.g., MYC. Any molecule or compound that specifically binds a DNA sequence may be used as a targeting moiety.

In some embodiments, a targeting moiety targets, e.g., binds, a component of a genomic complex (e.g., ASMC). In some embodiments, a targeting moiety targets, e.g., binds, an expression control sequence (e.g., a promoter or enhancer) operably linked to a target gene. In some embodiments, a targeting moiety targets, e.g., binds, a target gene or a part of a target gene. The target of a targeting moiety may be referred to as its targeted component. A targeted component may be any genomic sequence element operably linked to a target gene, or the target gene itself, including but not limited to a promoter, enhancer, anchor sequence, exon, intron, UTR encoding sequence, a splice site, or a transcription start site. In some embodiments, a targeting moiety binds specifically to one or more target anchor sequences (e.g., within a cell) and not to non-targeted anchor sequences (e.g., within the same cell).

In some embodiments, a targeting moiety may be or comprise a CRISPR/Cas domain, a TAL effector domain, a Zn finger domain, peptide nucleic acid (PNA) or a nucleic acid molecule. In some embodiments, an expression repressor comprises one effector moiety. In some embodiments, an expression repressor comprises a plurality of targeting moieties, wherein each targeting moiety does not detectably bind, e.g., does not bind, to another targeting moiety. In some embodiments, an expression repression system comprises a plurality of expression repressors, wherein each member of the plurality of expression repressors comprises a targeting moiety, wherein each targeting moiety does not detectably bind, e.g., does not bind, to another targeting moiety. In some embodiments, an expression repression system comprises a first expression repressor comprising a first targeting moiety and a second expression repressor comprising a second targeting moiety, wherein the first targeting moiety does not detectably bind, e.g., does not bind, to the second targeting moiety. In some embodiments, an expression repression system comprises a first expression repressor comprising a first targeting moiety and a second expression repressor comprising a second targeting moiety, wherein the first targeting moiety does not detectably bind, e.g., does not bind, to another first targeting moiety, and the second targeting moiety does not detectably bind, e.g., does not bind, to another second targeting moiety. In some embodiments, a targeting moiety for use in the compositions and methods described herein is functional (e.g., binds to a DNA sequence) in a monomeric, e.g., non-dimeric, state.

In some embodiments, binding of a targeting moiety to a targeted component decreases binding affinity of the targeted component for another transcription factor, genomic complex component, or genomic sequence element. In some embodiments, a targeting moiety binds to its target sequence with a K_{D} of less than or equal to 500, 450, 400, 350, 300, 250, 200, 150, 100, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM (and optionally, a K_{D} of at least 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM). In some embodiments, a targeting moiety binds to its target sequence with a K_{D} of 0.001 nM to 500 nM, e.g., 0.1 nM to 5 nM, e.g., about 0.5 nM. In some embodiments, a targeting moiety binds to a non-target sequence with a K_{D} of at least 500, 600, 700, 800, 900, 1000, 2000, 5000, 10,000, or 100,000 nM (and optionally, does not appreciably bind to a non-target sequence). In some embodiments, a targeting moiety does not bind to a non-target sequence.

In some embodiments, a targeting moiety comprises a nucleic acid sequence complementary to a targeted component, e.g., a regulatory element (e.g., promoter or enhancer) of a target gene, e.g., MYC. In some embodiments, a targeting moiety comprises a nucleic acid sequence that is at least 10%, 15%, 20%, 25%, 30%, 35%, 40%, 45%, 50%, 55%, 60%, 65%, 70%, 75%, 80%, 85%, 90%, 95%, 99%, or 100% complementary to a targeted component.

In some embodiments, a targeting moiety may be or comprise a CRISPR/Cas domain, a TAL effector domain, a Zn finger domain, or a nucleic acid molecule.

In some embodiments, the targeting moiety of an expression repressor comprises no more than 100, 90, 80, 70, 60, 50, 40, 30, or 20 nucleotides (and optionally at least 10, 20, 30, 40, 50, 60, 70, 80, or 90 nucleotides). In some embodiments, an expression repressor or the effector moiety of a fusion molecule, comprises no more than 2000, 1900, 1800, 1700, 1600, 1500, 1400, 1300, 1200, 1100, 1000, 900, 800, 700, 600, 500, 400, 300, 200, or 100 amino acids (and optionally at least 50, 100, 200, 300, 400, 500, 600, 700, 800, 900, 1000, 1100, 1200, 1300, 1400, 1500, 1600, 1700, 1800, or 1900 amino acids). In some embodiments, an expression repressor or the effector moiety of a fusion molecule, comprises 100-2000, 100-1900, 100-1800, 100-1700, 100-1600, 100-1500, 100-1400, 100-1300, 100-1200, 100-1100, 100-1000, 100-900, 100-800, 100-700, 100-600, 100-500, 100-400, 100-300, 100-200, 200-2000, 200-1900, 200-1800, 200-1700, 200-1600, 200-1500, 200-1400, 200-1300, 200-1200, 200-1100, 200-1000, 200-900, 200-800, 200-700, 200-600, 200-500, 200-400, 200-300, 300-2000, 300-1900, 300-1800, 300-1700, 300-1600, 300-1500, 300-1400, 300-1300, 300-1200, 300-1100, 300-1000, 300-900, 300-800, 300-700, 300-600, 300-500, 300-400, 400-2000, 400-1900, 400-1800, 400-1700, 400-1600, 400-1500, 400-1400, 400-1300, 400-1200, 400-1100, 400-1000, 400-900, 400-800, 400-700, 400-600, 400-500, 500-2000, 500-1900, 500-1800, 500-1700, 500-1600, 500-1500, 500-1400, 500-1300, 500-1200, 500-1100, 500-1000, 500-900, 500-800, 500-700, 500-600, 600-2000, 600-1900, 600-1800, 600-1700, 600-1600, 600-1500, 600-1400, 600-1300, 600-1200, 600-1100, 600-1000, 600-900, 600-800, 600-700, 700-2000, 700-1900, 700-1800, 700-1700, 700-1600, 700-1500, 700-1400, 700-1300, 700-1200, 700-1100, 700-1000, 700-900, 700-800, 800-2000, 800-1900, 800-1800, 800-1700, 800-1600, 800-1500, 800-1400, 800-1300, 800-1200, 800-1100, 800-1000, 800-900, 900-2000, 900-1900, 900-1800, 900-1700, 900-1600, 900-1500, 900-1400, 900-1300, 900-1200, 900-1100, 900-1000, 1000-2000, 1000-1900, 1000-1800, 1000-1700, 1000-1600, 1000-1500, 1000-1400, 1000-1300, 1000-1200, 1000-1100, 1100-2000, 1100-1900, 1100-1800, 1100-1700, 1100-1600, 1100-1500, 1100-1400, 1100-1300, 1100-1200, 1200-2000, 1200-1900, 1200-1800, 1200-1700, 1200-1600, 1200-1500, 1200-1400, 1200-1300, 1300-2000, 1300-1900, 1300-1800, 1300-1700, 1300-1600, 1300-1500, 1300-1400, 1400-2000, 1400-1900, 1400-1800, 1400-1700, 1400-1600, 1400-1500, 1500-2000, 1500-1900, 1500-1800, 1500-1700, 1500-1600, 1600-2000, 1600-1900, 1600-1800, 1600-1700, 1700-2000, 1700-1900, 1700-1800, 1800-2000, 1800-1900, or 1900-2000 amino acids.

An expression repressor or a system comprising an expressor as disclosed herein, may comprise nucleic acid, e.g., one or more nucleic acids. The term “nucleic acid” refers to any compound that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is or comprises more than 50% ribonucleotides and is referred to herein as a ribonucleic acid (RNA). In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. As used herein, “recombinant” when used to describe a nucleic acid refers to any nucleic acid that does not naturally occur. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, nucleic acids may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

In some embodiments, the targeting moiety comprises or is a nucleic acid sequence, a protein, protein fusion, or a membrane translocating polypeptide. In some embodiments, the targeting moiety is selected from an exogenous conjunction nucleating molecule, a nucleic acid encoding the conjunction nucleating molecule, or a fusion of a sequence targeting polypeptide and a conjunction nucleating molecule. The conjunction nucleating molecule may be, e.g., CTCF, cohesin, USF1, YY1, TATA-box binding protein associated factor 3 (TAF3), ZNF143 binding motif. In some embodiments, a targeting moiety comprises or is a polymer or polymeric moiety, e.g., a polymer of nucleotides (such as an oligonucleotide), a peptide nucleic acid, a peptide-nucleic acid mixmer, a peptide or polypeptide, a polyamide, a carbohydrate, etc.

In some embodiments, a targeting moiety comprises or is nucleic acid. In some embodiments, an effector moiety comprises or is nucleic acid. In some embodiments, a nucleic acid that may be included in a moiety may be or comprise DNA, RNA, and/or an artificial or synthetic nucleic acid or nucleic acid analog or mimic. For example, in some embodiments, a nucleic acid may be or include one or more of genomic DNA (gDNA), complementary DNA (cDNA), a peptide nucleic acid (PNA), a peptide-nucleic acid mixmer, a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with a targeted genomic complex as described herein), etc. A nucleic acid sequence suitable for use in a modulating agent may include modified oligonucleotides (e.g., chemical modifications, such as modifications that alter backbone linkages, sugar molecules, and/or nucleic acid bases) and/or artificial nucleic acids. In some embodiments, a nucleic acid sequence includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules. In some embodiments, a nucleic acid may include one or more residues that is not a naturally-occurring DNA or RNA residue, may include one or more linkages that is/are not phosphodiester bonds (e.g., that may be, for example, phosphorothioate bonds, etc.), and/or may include one or more modifications such as, for example, a 2′O modification such as 2′-OmeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

Some examples of nucleic acids include, but are not limited to, a nucleic acid that hybridizes to an target gene, e.g., MYC, (e.g., gRNA or antisense ssDNA as described herein elsewhere), a nucleic acid that hybridizes to an exogenous nucleic acid such as a viral DNA or RNA, nucleic acid that hybridizes to an RNA, a nucleic acid that interferes with gene transcription, a nucleic acid that interferes with RNA translation, a nucleic acid that stabilizes RNA or destabilizes RNA such as through targeting for degradation, a nucleic acid that interferes with a DNA or RNA binding factor through interference of its expression or its function, a nucleic acid that is linked to a intracellular protein or protein complex and modulates its function, etc.

In some embodiments, an expression repressor comprises one or more nucleoside analogs. In some embodiments, a nucleic acid sequence may include in addition or as an alternative to one or more natural nucleosides nucleosides, e.g., purines or pyrimidines, e.g., adenine, cytosine, guanine, thymine and uracil, one or more nucleoside analogs. In some embodiments, a nucleic acid sequence includes one or more nucleoside analogs. A nucleoside analog may include, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-β]pyridine, and any others that can base pair with a purine or a pyrimidine side chain.

### CRISPR/Cas Domains

In some embodiments, a targeting moiety is or comprises a CRISPR/Cas domain. A CRISPR/Cas protein can comprise a CRISPR/Cas effector and optionally one or more other domains. A CRISPR/Cas domain typically has structural and/or functional similarity to a protein involved in the clustered regulatory interspaced short palindromic repeat (CRISPR) system, e.g., a Cas protein. The CRISPR/Cas domain optionally comprises a guide RNA, e.g., single guide RNA (sgRNA). In some embodiments, the gRNA comprised by the CRISPR/Cas domain is noncovalently bound by the CRISPR/Cas domain.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. For example, in a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a partially double-stranded structure which is cleaved by Rnase III, resulting in a crRNA/tracrRNA hybrid. A crRNA/tracrRNA hybrid then directs Cas9 endonuclease to recognize and cleave a target DNA sequence. A target DNA sequence must generally be adjacent to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system requires only Cpf1 nuclease and a crRNA to cleave a target DNA sequence. Cpf1 endonucleases, are associated with T-rich PAM sites, e. g., 5′-TTN. Cpf1 can also recognize a 5′-CTA PAM motif. Cpf1 cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a DNA-targeting moiety includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a gram-positive bacteria or a gram-negative bacteria. In certain embodiments, a Cas protein may be from a Streptococcus (e.g., a S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.

In some embodiments, a Cas protein requires a protospacer adjacent motif (PAM) to be present in or adjacent to a target DNA sequence for the Cas protein to bind and/or function. In some embodiments, the PAM is or comprises, from 5′ to 3′, NGG, YG, NNGRRT, NNNRRT, NGA, TYCV, TATV, NTTN, or NNNGATT, where N stands for any nucleotide, Y stands for C or T, R stands for A or G, and V stands for A or C or G. In some embodiments, a Cas protein is a protein listed in Table 1. In some embodiments, a Cas protein comprises one or more mutations altering its PAM. In some embodiments, a Cas protein comprises E1369R, E1449H, and R1556A mutations or analogous substitutions to the amino acids corresponding to said positions. In some embodiments, a Cas protein comprises E782K, N968K, and R1015H mutations or analogous substitutions to the amino acids corresponding to said positions. In some embodiments, a Cas protein comprises D1135V, R1335Q, and T1337R mutations or analogous substitutions to the amino acids corresponding to said positions. In some embodiments, a Cas protein comprises S542R and K607R mutations or analogous substitutions to the amino acids corresponding to said positions. In some embodiments, a Cas protein comprises S542R, K548V, and N552R mutations or analogous substitutions to the amino acids corresponding to said positions.

TABLE 1

| | | | # of | | Mutations to alter | Mutations to make |
|---|---|---|---|---|---|---|
| Name | Enzyme | Species | Aas | PAM | PAM recognition | catalytically dead |
| FnCas9 | Cas9 | Francisella | 1629 | 5′-NGG-3′ | Wt | D11A/H969A/ |
| | | novicida | | | | N995A |
| FnCas9 | Cas9 | Francisella | 1629 | 5′-YG-3′ | E1369R/E1449H/ | D11A/H969A/ |
| RHA | | novicida | | | R1556A | N995A |
| SaCas9 | Cas9 | Staphylococcus | 1053 | 5′-NNGRRT-3′ | Wt | D10A/H557A |
| | | aureus | | | | |
| SaCas9 | Cas9 | Staphylococcus | 1053 | 5′-NNNRRT-3′ | E782K/N968K/ | D10A/H557A |
| KKH | | aureus | | | R1015H | |
| SpCas9 | Cas9 | Streptococcus | 1368 | 5′-NGG-3′ | Wt | D10A/D839A/ |
| | | pyogenes | | | | H840A/N863A |
| SpCas9 | Cas9 | Streptococcus | 1368 | 5′-NGA-3′ | D1135V/R1335Q/ | D10A/D839A/ |
| VQR | | pyogenes | | | T1337R | H840A/N863A |
| AsCpf1 | Cpf1 | Acidaminococcus | 1307 | 5′-TYCV-3′ | S542R/K607R | E993A |
| RR | | sp. BV3L6 | | | | |
| AsCpf1 | Cpf1 | Acidaminococcus | 1307 | 5′-TATV-3′ | S542R/K548V/ | E993A |
| RVR | | sp. BV3L6 | | | N552R | |
| FnCpf1 | Cpf1 | Francisella | 1300 | 5′NTTN-3′ | Wt | D917A/E1006A/ |
| | | novicida | | | | D1255A |
| NmCas9 | Cas9 | Neisseria | 1082 | 5′-NNNGATT-3′ | Wt | D16A/D587A/ |
| | | meningitidis | | | | H588A/N611A |

In some embodiments, the Cas protein is modified to deactivate the nuclease, e.g., nuclease-deficient Cas. In some embodiments, the Cas protein is a Cas9 protein. Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA. In some embodiments, dCas binding to a DNA sequence may interfere with transcription at that site by steric hindrance. In some embodiments, a DNA-targeting moiety is or comprises a catalytically inactive Cas, e.g., dCas. Many catalytically inactive Cas proteins are known in the art. In some embodiments, dCas9 comprises mutations in each endonuclease domain of the Cas protein, e.g., D10A and H840A mutations.

In some embodiments, a catalytically inactive Cas9 protein, e.g., dCas9, comprises a D11A mutation or an analogous substitution to the amino acid corresponding to said position. In some embodiments, a catalytically inactive Cas9 protein, e.g., dCas9, comprises a H969A mutation or an analogous substitution to the amino acid corresponding to said position. In some embodiments, a catalytically inactive Cas9 protein, e.g., dCas9, comprises a N995A mutation or an analogous substitution to the amino acid corresponding to said position. In some embodiments, a catalytically inactive Cas9 protein, e.g., dCas9, comprises D11A, H969A, and N995A mutations or analogous substitutions to the amino acids corresponding to said positions.

In some embodiments, a catalytically inactive Cas9 protein, e.g., dCas9, comprises a D10A mutation or an analogous substitution to the amino acid corresponding to said position. In some embodiments, a catalytically inactive Cas9 protein, e.g., dCas9, comprises a H557A mutation or an analogous substitution to the amino acid corresponding to said position. In some embodiments, a catalytically inactive Cas9 protein, e.g., dCas9, comprises D10A and H557A mutations or analogous substitutions to the amino acids corresponding to said positions.

In some embodiments, a catalytically inactive Cas9 protein, e.g., dCas9, comprises a D839A mutation or an analogous substitution to the amino acid corresponding to said position. In some embodiments, a catalytically inactive Cas9 protein, e.g., dCas9, comprises a H840A mutation or an analogous substitution to the amino acid corresponding to said position. In some embodiments, a catalytically inactive Cas9 protein, e.g., dCas9, comprises a N863A mutation or an analogous substitution to the amino acid corresponding to said position. In some embodiments, a catalytically inactive Cas9 protein, e.g., dCas9, comprises D10A, D839A, H840A, and N863A mutations or analogous substitutions to the amino acids corresponding to said positions.

In some embodiments, a catalytically inactive Cas9 protein, e.g., dCas9, comprises a E993A mutation or an analogous substitution to the amino acid corresponding to said position.

In some embodiments, a catalytically inactive Cas9 protein, e.g., dCas9, comprises a D917A mutation or an analogous substitution to the amino acid corresponding to said position. In some embodiments, a catalytically inactive Cas9 protein, e.g., dCas9, comprises a E1006A mutation or an analogous substitution to the amino acid corresponding to said position. In some embodiments, a catalytically inactive Cas9 protein, e.g., dCas9, comprises a D1255A mutation or an analogous substitution to the amino acid corresponding to said position. In some embodiments, a catalytically inactive Cas9 protein, e.g., dCas9, comprises D917A, E1006A, and D1255A mutations or analogous substitutions to the amino acids corresponding to said positions.

In some embodiments, a catalytically inactive Cas9 protein, e.g., dCas9, comprises a D16A mutation or an analogous substitution to the amino acid corresponding to said position. In some embodiments, a catalytically inactive Cas9 protein, e.g., dCas9, comprises a D587A mutation or an analogous substitution to the amino acid corresponding to said position. In some embodiments, a catalytically inactive Cas9 protein, e.g., dCas9, comprises a H588A mutation or an analogous substitution to the amino acid corresponding to said position. In some embodiments, a catalytically inactive Cas9 protein, e.g., dCas9, comprises a N611A mutation or an analogous substitution to the amino acid corresponding to said position. In some embodiments, a catalytically inactive Cas9 protein, e.g., dCas9, comprises D16A, D587A, H588A, and N611A mutations or analogous substitutions to the amino acids corresponding to said positions.

In another aspect, the disclosure is directed to an expression repressor or a polypeptide comprising one or more (e.g., one) targeting moiety and one or more effector moiety, wherein the one or more targeting moiety is or comprises a CRISPR/Cas domain comprising a Cas protein, e.g., catalytically inactive Cas9 protein, e.g., dCas9, or a functional variant or fragment thereof. In some embodiments, dCas9 comprises an amino acid sequence of SEQ ID NO: 17:

(SEQ ID NO: 17)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVAAIVPQSFLKDDS
IDNKVLTRSDKARGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

In some embodiments, the dCas9 is encoded by a nucleic acid sequence of SEQ ID NO: 50:

(SEQ ID NO: 50)
GACAAGAAGTACAGCATCGGCCTGGCCATCGGCACCAACAGCGTGGGCTGGGCCGT
GATCACCGACGAGTACAAGGTGCCCAGCAAGAAGTTCAAGGTGCTGGGCAACACCGACCGG
CACAGCATCAAGAAGAACCTGATCGGCGCCCTGCTGTTCGACAGCGGCGAGACCGCCGAGG
CCACCCGGCTGAAGCGGACCGCCCGGCGGCGGTACACCCGGCGGAAGAACCGGATCTGCTA
CCTGCAGGAGATCTTCAGCAACGAGATGGCCAAGGTGGACGACAGCTTCTTCCACCGGCTG
GAGGAGAGCTTCCTGGTGGAGGAGGACAAGAAGCACGAGCGGCACCCCATCTTCGGCAACA
TCGTGGACGAGGTGGCCTACCACGAGAAGTACCCCACCATCTACCACCTGCGGAAGAAGCT
GGTGGACAGCACCGACAAGGCCGACCTGCGGCTGATCTACCTGGCCCTGGCCCACATGATC
AAGTTCCGGGGCCACTTCCTGATCGAGGGCGACCTGAACCCCGACAACAGCGACGTGGACA
AGCTGTTCATCCAGCTGGTGCAGACCTACAACCAGCTGTTCGAGGAGAACCCCATCAACGCC
AGCGGCGTGGACGCCAAGGCCATCCTGAGCGCCCGGCTGAGCAAGAGCCGGCGGCTGGAGA
ACCTGATCGCCCAGCTGCCCGGCGAGAAGAAGAACGGCCTGTTCGGCAACCTGATCGCCCT
GAGCCTGGGCCTGACCCCCAACTTCAAGAGCAACTTCGACCTGGCCGAGGACGCCAAGCTG
CAGCTGAGCAAGGACACCTACGACGACGACCTGGACAACCTGCTGGCCCAGATCGGCGACC
AGTACGCCGACCTGTTCCTGGCCGCCAAGAACCTGAGCGACGCCATCCTGCTGAGCGACATC
CTGCGGGTGAACACCGAGATCACCAAGGCCCCCCTGAGCGCCAGCATGATCAAGCGGTACG
ACGAGCACCACCAGGACCTGACCCTGCTGAAGGCCCTGGTGCGGCAGCAGCTGCCCGAGAA
GTACAAGGAGATCTTCTTCGACCAGAGCAAGAACGGCTACGCCGGCTACATCGACGGCGGC
GCCAGCCAGGAGGAGTTCTACAAGTTCATCAAGCCCATCCTGGAGAAGATGGACGGCACCG
AGGAGCTGCTGGTGAAGCTGAACCGGGAGGACCTGCTGCGGAAGCAGCGGACCTTCGACAA
CGGCAGCATCCCCCACCAGATCCACCTGGGCGAGCTGCACGCCATCCTGCGGCGGCAGGAG
GACTTCTACCCCTTCCTGAAGGACAACCGGGAGAAGATCGAGAAGATCCTGACCTTCCGGAT
CCCCTACTACGTGGGCCCCCTGGCCCGGGGCAACAGCCGGTTCGCCTGGATGACCCGGAAAT
CCGAGGAGACCATCACCCCCTGGAACTTCGAGGAGGTGGTGGACAAGGGCGCCAGCGCCCA
GAGCTTCATCGAGCGGATGACCAACTTCGACAAGAACCTGCCCAACGAGAAGGTGCTGCCC
AAGCACAGCCTGCTGTACGAGTACTTCACCGTGTACAACGAGCTGACCAAGGTGAAGTACG
TGACCGAGGGCATGCGGAAGCCCGCCTTCCTGAGCGGCGAGCAGAAGAAGGCCATCGTGGA
CCTGCTGTTCAAGACCAACCGGAAGGTGACCGTGAAGCAGCTGAAGGAGGACTACTTCAAG
AAGATCGAGTGCTTCGACAGCGTGGAGATCAGCGGCGTGGAGGACCGGTTCAACGCCAGCC
TGGGCACCTACCACGACCTGCTGAAGATCATCAAGGACAAGGACTTCCTGGACAACGAGGA
GAACGAGGACATCCTGGAGGACATCGTGCTGACCCTGACCCTGTTCGAGGACCGGGAGATG
ATCGAGGAGCGGCTGAAAACCTACGCCCACCTGTTCGACGACAAGGTGATGAAGCAGCTGA
AGCGGCGGCGGTACACCGGCTGGGGCCGGCTGAGCCGGAAGCTGATCAACGGCATCCGGGA
CAAGCAGAGCGGCAAGACCATCCTGGACTTCCTGAAATCCGACGGCTTCGCCAACCGGAAC
TTCATGCAGCTGATCCACGACGACAGCCTGACCTTCAAGGAGGACATCCAGAAGGCCCAGG
TGAGCGGCCAGGGCGACAGCCTGCACGAGCACATCGCCAACCTGGCCGGCAGCCCCGCCAT
CAAGAAGGGCATCCTGCAGACCGTGAAGGTGGTGGACGAGCTGGTGAAGGTGATGGGCCGG
CACAAGCCCGAGAACATCGTGATCGAGATGGCCCGGGAGAACCAGACCACCCAGAAGGGCC
AGAAGAACAGCCGGGAGCGGATGAAGCGGATCGAGGAGGGCATCAAGGAGCTGGGCAGCC
AGATCCTGAAGGAGCACCCCGTGGAGAACACCCAGCTGCAGAACGAGAAGCTGTACCTGTA
CTACCTGCAGAACGGCCGGGACATGTACGTGGACCAGGAGCTGGACATCAACCGGCTGAGC
GACTACGACGTGGCCGCCATCGTGCCCCAGAGCTTCCTGAAGGACGACAGCATCGACAACA
AGGTGCTGACCCGGAGCGACAAGGCCCGGGGCAAGAGCGACAACGTGCCCAGCGAGGAGG
TGGTGAAGAAGATGAAGAACTACTGGCGGCAGCTGCTGAACGCCAAGCTGATCACCCAGCG
GAAGTTCGACAACCTGACCAAGGCCGAGCGGGGCGGCCTGAGCGAGCTGGACAAGGCCGGC
TTCATCAAGCGGCAGCTGGTGGAGACCCGGCAGATCACCAAGCACGTGGCCCAGATCCTGG
ACAGCCGGATGAACACCAAGTACGACGAGAACGACAAGCTGATCCGGGAGGTGAAGGTGA
TCACCCTGAAATCCAAGCTGGTGAGCGACTTCCGGAAGGACTTCCAGTTCTACAAGGTGCGG
GAGATCAACAACTACCACCACGCCCACGACGCCTACCTGAACGCCGTGGTGGGCACCGCCC
TGATCAAGAAGTACCCCAAGCTGGAGAGCGAGTTCGTGTACGGCGACTACAAGGTGTACGA
CGTGCGGAAGATGATCGCCAAGAGCGAGCAGGAGATCGGCAAGGCCACCGCCAAGTACTTC
TTCTACAGCAACATCATGAACTTCTTCAAGACCGAGATCACCCTGGCCAACGGCGAGATCCG
GAAGCGGCCCCTGATCGAGACCAACGGCGAGACCGGCGAGATCGTGTGGGACAAGGGCCG
GGACTTCGCCACCGTGCGGAAGGTGCTGAGCATGCCCCAGGTGAACATCGTGAAGAAAACC
GAGGTGCAGACCGGCGGCTTCAGCAAGGAGAGCATCCTGCCCAAGCGGAACAGCGACAAGC
TGATCGCCCGGAAGAAGGACTGGGACCCCAAGAAGTACGGCGGCTTCGACAGCCCCACCGT
GGCCTACAGCGTGCTGGTGGTGGCCAAGGTGGAGAAGGGCAAGAGCAAGAAGCTGAAATCC
GTGAAGGAGCTGCTGGGCATCACCATCATGGAGCGGAGCAGCTTCGAGAAGAACCCCATCG
ACTTCCTGGAGGCCAAGGGCTACAAGGAGGTGAAGAAGGACCTGATCATCAAGCTGCCCAA
GTACAGCCTGTTCGAGCTGGAGAACGGCCGGAAGCGGATGCTGGCCAGCGCCGGCGAGCTG
CAGAAGGGCAACGAGCTGGCCCTGCCCAGCAAGTACGTGAACTTCCTGTACCTGGCCAGCC
ACTACGAGAAGCTGAAGGGCAGCCCCGAGGACAACGAGCAGAAGCAGCTGTTCGTGGAGC
AGCACAAGCACTACCTGGACGAGATCATCGAGCAGATCAGCGAGTTCAGCAAGCGGGTGAT
CCTGGCCGACGCCAACCTGGACAAGGTGCTGAGCGCCTACAACAAGCACCGGGACAAGCCC
ATCCGGGAGCAGGCCGAGAACATCATCCACCTGTTCACCCTGACCAACCTGGGCGCCCCCGC
CGCCTTCAAGTACTTCGACACCACCATCGACCGGAAGCGGTACACCAGCACCAAGGAGGTG
CTGGACGCCACCCTGATCCACCAGAGCATCACCGGCCTGTACGAGACCCGGATCGACCTGA
GCCAGCTGGGCGGCGAC

In some embodiments, a targeting moiety may comprise a Cas domain comprising or linked (e.g., covalently) to a gRNA. A gRNA is a short synthetic RNA composed of a “scaffold” sequence necessary for Cas-protein binding and a user-defined ˜20 nucleotide targeting sequence for a genomic target. In practice, guide RNA sequences are generally designed to have a length of between 17-24 nucleotides (e.g., 19, 20, or 21 nucleotides) and be complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective for use with Cas proteins; see, for example, Hendel et al. (2015) Nature Biotechnol., 985-991. The exemplary guide RNA sequences are disclosed in Table 2 and Table 13.

In some embodiments, a gRNA comprises a nucleic acid sequence that is complementary to a DNA sequence associated with a target gene. In some embodiments, the DNA sequence is, comprises, or overlaps an expression control element that is operably linked to the target gene. In some embodiments, a gRNA comprises a nucleic acid sequence that is at least 90, 95, 99, or 100% complementary to a DNA sequence associated with a target gene. In some embodiments, a gRNA for use with a DNA-targeting moiety that comprises a Cas molecule is an sgRNA.

In some embodiments, a gRNA for use with a CRISPR/Cas domain specifically binds a target sequence associated with CTCF. In some embodiments, a gRNA for use with a CRISPR/Cas domain specifically binds a target sequence associated with the promoter. In some embodiments, the gRNA binds a target sequence listed in Table 2 or Table 13. In some embodiments, an expression repressor described herein binds to a target sequence listed in Table 2 or Table 13.

TABLE 2
Exemplary gRNA sequences

| | | | Genomic | |
|---|---|---|---|---|
| Guide Name | Target Site | Target Sequence | Coordinates | Strand |
| GD-28616 | CTCF | ATGATCTCTGCTGCCAGTAG | chr8: 128746342- | + |
| | | (SEQ ID NO: 1) | 128746364 | |
| GD-28859 | CTCF | ATCGCGCCTGGATGTCAACG | chr8: 128746321- | − |
| | | (SEQ ID NO: 2) | 128746343 | |
| GD-28862 | CTCF | ATTGTGCAGTGCATCGGATT | chr8: 128746525- | + |
| | | (SEQ ID NO: 3) | 128746547 | |
| GD-28617 | Promoter | GTCAAACAGTACTGCTACGG | chr8: 128748014- | + |
| | | (SEQ ID NO: 4) | 128748036 | |

TABLE 13
Exemplary gRNA sequences

| Guide Name | Target Sequence | Genomic Coordinates |
|---|---|---|
| GD-29833 | TGCCACTTCCCCACTAACCC | GRCh37: chr8: 129188878- |
| | (SEQ ID NO: 96) | 129188900 |
| GD-29834 | GGCCACACAAGGAAGCTGCA | GRCh37: chr8: 129188958- |
| | (SEQ ID NO: 97) | 129188980 |
| GD-29835 | CCACACAAGGAAGCTGCAGG | GRCh37: chr8: 129188960- |
| | (SEQ ID NO: 98) | 129188982 |
| GD-29836 | TGATTGGAATGCAACCCGAA | GRCh37: chr8: 129189067- |
| | (SEQ ID NO: 99) | 129189089 |
| GD-29837 | TTTTGCCCTTGCTACCCCAA | GRCh37: chr8: 129189457- |
| | (SEQ ID NO: 100) | 129189479 |
| GD-29838 | AGCTGATGGTATCCACTAGG | GRCh37: chr8: 129189554- |
| | (SEQ ID NO: 101) | 129189576 |
| GD-29839 | CACATCCAAGAATGTAGTGG | GRCh37: chr8: 129189679- |
| | (SEQ ID NO: 102) | 129189701 |
| GD-29840 | GATACAGCCACAAAGCTCAC | GRCh37: chr8: 129209511- |
| | (SEQ ID NO: 103) | 129209533 |
| GD-29841 | ATTACATAACAGAATCCAGG | GRCh37: chr8: 129209643- |
| | (SEQ ID NO: 104) | 129209665 |
| GD-29842 | CCCTTGACTGTGCTGCCACC | GRCh37: chr8: 129209658- |
| | (SEQ ID NO: 105) | 129209680 |
| GD-29843 | CAGACGAGGAACCTGAACCC | GRCh37: chr8: 129209856- |
| | (SEQ ID NO: 106) | 129209878 |
| GD-29844 | AGAATCCCTTGGGGTAGCAA | GRCh37: chr8: 129189452- |
| | (SEQ ID NO: 107) | 129189474 |
| GD-29914 | CAGCACTCTCGCTGACCGCA | GRCh37: chr8: 129189190- |
| | (SEQ ID NO: 108) | 129189212 |
| GD-29915 | GTTGAGTCATGTGTACTCTG | GRCh37: chr8: 129189274- |
| | (SEQ ID NO: 109) | 129189296 |
| GD-29916 | AGGAACAGGATGTTACAACT | GRCh37: chr8: 129189421- |
| | (SEQ ID NO: 110) | 129189443 |
| GD-28662 | GGGGCCACTAGGGACAGGAT | GRCh37: chr19: 55627120- |
| | (SEQ ID NO: 111) | 55627139 |

In some embodiments, an expression repression system comprises a first expression repressor comprising a first DNA-targeting moiety and a second expression repressor comprising a second DNA-targeting moiety, wherein the first DNA-targeting moiety comprises or is a first CRISPR/Cas domain and the second DNA-targeting moiety comprises or is a second CRISPR/Cas domain. In some embodiments, the first CRISPR/Cas domain comprises a first CRISPR/Cas protein and first guide RNA, and the second CRISPR/Cas domain comprises a second CRISPR/Cas protein and a second guide RNA. In some embodiments, the first CRISPR/Cas protein does not appreciably bind (e.g., does not bind) the second guide RNA, e.g., binds with a K_{D} of at least 10, 20, 50, 100, 1000, or 10,000 nM, and the second CRISPR/Cas protein does not appreciably bind (e.g., does not bind) the first guide RNA, e.g., binds with a K_{D} of at least 10, 20, 50, 100, 1000, or 10,000 nM.

### TAL Effector Domains

In some embodiments, a DNA-targeting moiety is or comprises a TAL effector domain. A TAL effector domain, e.g., a TAL effector domain that specifically binds a DNA sequence, comprises a plurality of TAL effector repeats or fragments thereof, and optionally one or more additional portions of naturally occurring TAL effector repeats (e.g., N- and/or C-terminal of the plurality of TAL effector domains) wherein each TAL effector repeat recognizes a nucleotide. A TAL effector protein can comprise a TAL effector domain and optionally one or more other domains. Many TAL effector domains are known to those of skill in the art and are commercially available, e.g., from Thermo Fisher Scientific.

TALEs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat-variable di-residues, RVD domain).

Members of the TAL effectors family differ mainly in the number and order of their repeats. The number of repeats ranges from 1.5 to 33.5 repeats and the C-terminal repeat is usually shorter in length (e.g., about 20 amino acids) and is generally referred to as a “half-repeat”. Each repeat of the TAL effector features a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 3 listing exemplary repeat variable di-residues (RVD) and their correspondence to nucleic acid base targets.

TABLE 3
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, the TAL effector repeat of the TAL effector domain of the present disclosure may be derived from a TAL effector from any bacterial species (e.g., Xanthomonas species such as the African strain of Xanthomonas oryzae pv. Oryzae (Yu et al. 2011), Xanthomonas campestris pv. raphani strain strain 756C and Xanthomonas oryzae pv. Oryzicolastrain BLS256 (Bogdanove et al. 2011). As used herein, the TAL effector domain in accordance with the present disclosure comprises an RVD domain as well as flanking sequence(s) (sequences on the N-terminal and/or C-terminal side of the RVD domain) also from the naturally occurring TAL effector. It may comprise more or fewer repeats than the RVD of the naturally occurring TAL effector domain. The TAL effector domain of the present disclosure is designed to target a given DNA sequence based on the above code and others known in the art. The number of TAL effector repeats (e.g., monomers or modules) and their specific sequence are selected based on the desired DNA target sequence. For example, TAL effector repeats, may be removed or added in order to suit a specific target sequence. In an embodiment, the TAL effector domain of the present disclosure comprises between 6.5 and 33.5 TAL effector repeats. In an embodiment, TAL effector domain of the present disclosure comprises between 8 and 33.5 TAL effector repeats, e.g., between 10 and 25 TAL effector repeats, e.g., between 10 and 14 TAL effector repeats.

In some embodiments, the TAL effector domain comprises TAL effector repeats that correspond to a perfect match to the DNA target sequence. In some embodiments, a mismatch between a repeat and a target base-pair on the DNA target sequence is permitted as along as it allows for the function of the expression repression system, e.g., the expression repressor comprising the TAL effector domain. In general, TALE binding is inversely correlated with the number of mismatches. In some embodiments, the TAL effector domain of a expression repressor of the present disclosure comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector repeats in the TAL effector domain, the smaller the number of mismatches will be tolerated and still allow for the function of the expression repression system, e.g., the expression repressor comprising the TAL effector domain. The binding affinity is thought to depend on the sum of matching repeat-DNA combinations. For example, TAL effector domains having 25 TAL effector repeats or more may be able to tolerate up to 7 mismatches.

In addition to the TAL effector repeats, the TAL effector domain of the present disclosure may comprise additional sequences derived from a naturally occurring TAL effector. The length of the C-terminal and/or N-terminal sequence(s) included on each side of the TAL effector repeat portion of the TAL effector domain can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector repeats of the naturally occurring TAL effector is included in the TAL effector domain of an expression repressor of the present disclosure. Accordingly, in an embodiment, a TAL effector domain of the present disclosure comprises 1) one or more TAL effector repeats derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector repeats; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector repeats.

In some embodiments, a modulating agent comprises a targeting moiety comprising an engineered DNA binding domain (DBD), e.g., a TAL effector comprising a TAL effector repeat that binds to a target sequence, e.g., a promoter or transcription start site (TSS)) sequence operably linked to a target gene (e.g., MYC), e.g., a sequence proximal to the transcription regulatory element, e.g., an anchor sequence of an anchor sequence mediated conjunction (ASMC) comprising a target gene (e.g., MYC), e.g., a sequence proximal to the anchor sequence. In some embodiments, the TAL effector domain can be engineered to carry epigenetic effector moieties to target sites.

### Zn Finger Domains

In some embodiments, a DNA-targeting moiety is or comprises a Zn finger domain. A Zn finger domain comprises a Zn finger, e.g., a naturally occurring Zn finger or engineered Zn finger, or fragment thereof. Many Zn fingers are known to those of skill in the art and are commercially available, e.g., from Sigma-Aldrich. Generally, a Zn finger domain comprises a plurality of Zn fingers, wherein each Zn finger recognizes three nucleotides. A Zn finger protein can comprise a Zn finger domain and optionally one or more other domains.

In some embodiments, a Zn finger molecule comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger may have a novel binding specificity, compared to a naturally-occurring Zn finger. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc fingers and/or multi-fingered zinc finger domains may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

Zn fingers and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In certain embodiments, the DNA-targeting moiety comprises a Zn finger domain comprising an engineered zinc finger that binds (in a sequence-specific manner) to a target DNA sequence. In some embodiments, the Zn finger domain comprises one Zn finger or fragment thereof. In some embodiments, the Zn finger domain comprises a plurality of Zn fingers (or fragments thereof), e.g., 2, 3, 4, 5, 6 or more Zn fingers (and optionally no more than 12, 11, 10, 9, 8, 7, 6, 5, 4, 3, or 2 Zn fingers). In some embodiments, the Zn finger domain comprises at least three Zn fingers. In some embodiments, the Zn finger domain comprises four, five or six Zn fingers. In some embodiments, the Zn finger domain comprises 8, 9, 10, 11 or 12 Zn fingers. In some embodiments, a Zn finger domain comprising three Zn fingers recognizes a target DNA sequence comprising 9 or 10 nucleotides. In some embodiments, a Zn finger domain comprising four Zn fingers recognizes a target DNA sequence comprising 12 to 14 nucleotides. In some embodiments, a Zn finger domain comprising six Zn fingers recognizes a target DNA sequence comprising 18 to 21 nucleotides.

In some embodiments, a targeting domain comprises a two-handed Zn finger protein. Two handed zinc finger proteins are those proteins in which two clusters of zinc fingers are separated by intervening amino acids so that the two zinc finger domains bind to two discontinuous target DNA sequences. An example of a two-handed type of zinc finger binding protein is SIP1, where a cluster of four zinc fingers is located at the amino terminus of the protein and a cluster of three Zn fingers is located at the carboxyl terminus (see Remade, et al. (1999) EMBO Journal 18(18):5073-5084). Each cluster of zinc fingers in these domains is able to bind to a unique target sequence and the spacing between the two target sequences can comprise many nucleotides.

In some embodiments, an expression repressor comprises a targeting moiety comprising an engineered DNA binding domain (DBD), e.g., a Zn finger domain comprising a Zn finger (ZFN) that binds to a target sequence, e.g., a promoter or transcription start site (TSS)) sequence operably linked to a target gene (e.g., MYC), e.g., a sequence proximal to the transcription regulatory element, e.g., an anchor sequence of an anchor sequence mediated conjunction (ASMC) comprising a target gene (e.g., MYC), e.g., a sequence proximal to the anchor sequence. In some embodiments, the ZFN can be engineered to carry epigenetic effector molecules to target sites. In some embodiments, the targeting moiety comprises a Zn Finger domain that comprises 2, 3, 4, 5, 6, 7, or 8 zinc fingers. The amino acid sequences of exemplary targeting moieties disclosed herein are listed in Table 4. The nucleotide sequences encoding exemplary targeting moieties disclosed herein are listed in Table 5. In some embodiments, an expression repressor or system described herein comprises a targeting moiety having a sequence set forth in Table 4, or a sequence with at least 70%, 75%, 80%, 85%, 90%, 95%, 98%, or 99% identity thereto. In some embodiments, a nucleic acid described herein comprises a sequence set forth in Table 5, or a sequence with at least 70%, 75%, 80%, 85%, 90%, 95%, 98%, or 99% identity thereto.

TABLE 4
Amino acid sequences of exemplary targeting moieties

| | SEQ | |
|---|---|---|
| | ID | |
| NAME | NO. | SEQUENCE |
| ZF1 | 5 | LEPGEKPYKCPECGKSFSRSDKLTEHQRTHTGEKPYKCPECGKSFSTKNSLTEHQRTHTGEKPYKCPECGKSFSQ |
| (aa) | | SGDLRRHQRTHTGEKPYKCPECGKSFSTTGALTEHQRTHTGEKPYKCPECGKSFSDSGNLRVHQRTHTGEKPYK |
| | | CPECGKSFSQRAHLERHQRTHTGEKPYKCPECGKSFSTSGELVRHQRTHTGEKPTGKKTS |
| ZF2 | 6 | LEPGEKPYKCPECGKSFSSPADLTRHQRTHTGEKPYKCPECGKSFSDPGHLVRHQRTHTGEKPYKCPECGKSFSR |
| (aa) | | SDNLVRHQRTHTGEKPYKCPECGKSFSQSSSLVRHQRTHTGEKPYKCPECGKSFSTSHSLTEHQRTHTGEKPYK |
| | | CPECGKSFSRNDALTEHQRTHTGEKPYKCPECGKSFSRNDALTEHQRTHTGEKPTGKKTS |
| ZF3 | 7 | LEPGEKPYKCPECGKSFSRSDKLVRHQRTHTGEKPYKCPECGKSFSQRAHLERHQRTHTGEKPYKCPECGKSFS |
| (aa) | | DPGHLVRHQRTHTGEKPYKCPECGKSFSRSDKLVRHQRTHTGEKPYKCPECGKSFSQLAHLRAHQRTHTGEKP |
| | | YKCPECGKSFSRADNLTEHQRTHTGEKPYKCPECGKSFSDCRDLARHQRTHTGEKPTGKKTS |
| ZF4 | 8 | LEPGEKPYKCPECGKSFSHTGHLLEHQRTHTGEKPYKCPECGKSFSTTGNLTVHQRTHTGEKPYKCPECGKSF |
| (aa) | | SDKKDLTRHQRTHTGEKPYKCPECGKSFSQSGDLRRHQRTHTGEKPYKCPECGKSFSQRAHLERHQRTHTGE |
| | | KPYKCPECGKSFSRSDKLTEHQRTHTGEKPYKCPECGKSFSRTDTLRDHQRTHTGEKPTGKKTS |
| ZF5 | 9 | LEPGEKPYKCPECGKSFSHTGHLLEHQRTHTGEKPYKCPECGKSFSTSGNLTEHQRTHTGEKPYKCPECGKSFS |
| (aa) | | TSGNLVRHQRTHTGEKPYKCPECGKSFSQLAHLRAHQRTHTGEKPYKCPECGKSFSERSHLREHQRTHTGEKP |
| | | YKCPECGKSFSDPGHLVRHQRTHTGEKPYKCPECGKSFSTHLDLIRHQRTHTGEKPTGKKTS |
| ZF6 | 10 | LEPGEKPYKCPECGKSFSDPGALVRHQRTHTGEKPYKCPECGKSFSERSHLREHQRTHTGEKPYKCPECGKSF |
| (aa) | | SRSDHLTNHQRTHTGEKPYKCPECGKSFSERSHLREHQRTHTGEKPYKCPECGKSFSRSDHLTNHQRTHTGEK |
| | | PYKCPECGKSFSDPGHLVRHQRTHTGEKPYKCPECGKSFSRSDKLVRHQRTHTGEKPTGKKTS |
| ZF7 | 11 | LEPGEKPYKCPECGKSFSRNDALTEHQRTHTGEKPYKCPECGKSFSDCRDLARHQRTHTGEKPYKCPECGKSF |
| (aa) | | SDPGHLVRHQRTHTGEKPYKCPECGKSFSQSGHLTEHQRTHTGEKPYKCPECGKSFSREDNLHTHQRTHTGEK |
| | | PYKCPECGKSFSTKNSLTEHQRTHTGEKPYKCPECGKSFSRADNLTEHQRTHTGEKPTGKKTS |
| ZF8 | 12 | LEPGEKPYKCPECGKSFSRSDKLTEHQRTHTGEKPYKCPECGKSFSRRDELNVHQRTHTGEKPYKCPECGKSF |
| (aa) | | SRSDHLTNHQRTHTGEKPYKCPECGKSFSSPADLTRHQRTHTGEKPYKCPECGKSFSRSDHLTNHQRTHTGEK |
| | | PYKCPECGKSFSSKKALTEHQRTHTGEKPYKCPECGKSFSTHLDLIRHQRTHTGEKPTGKKTS |
| ZF9 | 13 | LEPGEKPYKCPECGKSFSRSDDLVRHQRTHTGEKPYKCPECGKSFSREDNLHTHQRTHTGEKPYKCPECGKS |
| (aa) | | FSRSDHLTTHQRTHTGEKPYKCPECGKSFSQRANLRAHQRTHTGEKPYKCPECGKSFSQLAHLRAHQRTHTG |
| | | EKPYKCPECGKSFSQRANLRAHQRTHTGEKPYKCPECGKSFSERSHLREHQRTHTGEKPTGKKTS |
| ZF10 | 14 | LEPGEKPYKCPECGKSFSQSGDLRRHQRTHTGEKPYKCPECGKSFSQSGHLTEHQRTHTGEKPYKCPECGKSF |
| (aa) | | SERSHLREHQRTHTGEKPYKCPECGKSFSDPGHLVRHQRTHTGEKPYKCPECGKSFSRNDTLTEHQRTHTGEK |
| | | PYKCPECGKSFSRADNLTEHQRTHTGEKPYKCPECGKSFSTHLDLIRHQRTHTGEKPTGKKTS |
| ZF11 | 15 | LEPGEKPYKCPECGKSFSHTGHLLEHQRTHTGEKPYKCPECGKSFSSKKALTEHQRTHTGEKPYKCPECGKSFS |
| (aa) | | DCRDLARHQRTHTGEKPYKCPECGKSFSHTGHLLEHQRTHTGEKPYKCPECGKSFSRNDALTEHQRTHTGEKP |
| | | YKCPECGKSFSQSGDLRRHQRTHTGEKPYKCPECGKSFSDSGNLRVHQRTHTGEKPTGKKTS |
| ZF12 | 16 | LEPGEKPYKCPECGKSFSQSSSLVRHQRTHTGEKPYKCPECGKSFSRSDHLTNHQRTHTGEKPYKCPECGKSFS |
| (aa) | | QLAHLRAHQRTHTGEKPYKCPECGKSFSQSSNLVRHQRTHTGEKPYKCPECGKSFSRSDNLVRHQRTHTGEKP |
| | | YKCPECGKSFSRSDELVRHQRTHTGEKPYKCPECGKSFSQLAHLRAHQRTHTGEKPTGKKTS |
| ZF54 | 169 | LEPGEKPYKCPECGKSFSERSHLREHQRTHTGEKPYKCPECGKSFSRSDNLVRHQRTHTGEKPYKCPECGKSFS |
| (aa) | | DCRDLARHQRTHTGEKPYKCPECGKSFSTSGELVRHQRTHTGEKPYKCPECGKSFSTTGNLTVHQRTHTGEKP |
| | | YKCPECGKSFSRSDKLVRHQRTHTGEKPYKCPECGKSFSRTDTLRDHQRTHTGKKTS |
| ZF61 | 170 | LEPGEKPYKCPECGKSFSQKSSLIAHQRTHTGEKPYKCPECGKSFSHKNALQNHQRTHTGEKPYKCPECGKSFS |
| (aa) | | QSSNLVRHQRTHTGEKPYKCPECGKSFSRNDALTEHQRTHTGEKPYKCPECGKSFSDKKDLTRHQRTHTGEKP |
| | | YKCPECGKSFSQAGHLASHQRTHTGEKPYKCPECGKSFSDKKDLTRHORTHTGKKTS |
| ZF67 | 171 | LEPGEKPYKCPECGKSFSRSDNLVRHQRTHTGEKPYKCPECGKSFSDCRDLARHQRTHTGEKPYKCPECGKSF |
| (aa) | | STSGELVRHQRTHTGEKPYKCPECGKSFSTTGNLTVIIQRTIITGEKPYKCPECGKSFSRSDKLVRHQRTHTGEK |
| | | PYKCPECGKSFSRTDTLRDHQRTHTGEKPYKCPECGKSFSREDNLHTHQRTHTGKKTS |
| ZF68 | 172 | LEPGEKPYKCPECGKSFSRSDHLTTHQRTHTGEKPYKCPECGKSFSQKSSLIAHQRTHTGEKPYKCPECGKSFS |
| (aa) | | RRDELNVHQRTHTGEKPYKCPECGKSFSQSGDLRRHQRTHTGEKPYKCPECGKSFSQAGHLASHQRTHTGEK |
| | | PYKCPECGKSFSTSGNLTEHQRTHTGEKPYKCPECGKSFSQAGHLASHQRTHTGKKTS |

TABLE 5
Nucleotide sequences of exemplary targeting moieties

| | SEQ | |
|---|---|---|
| | ID | |
| NAME | NO. | SEQUENCE |
| ZF1 | 38 | CTGGAGCCCGGCGAGAAACCCTACAAGTGCCCCGAGTGCGGCAAATCCTTCTCTAGAAGCGACAAACTGAC |
| (nt) | | CGAACATCAGAGGACCCACACCGGCGAGAAGCCTTATAAGTGTCCCGAATGCGGCAAATCCTTCAGCACCA |
| | | AGAACTCTCTGACAGAACACCAGAGAACACATACCGGAGAGAAACCTTATAAATGCCCCGAGTGCGGCAAG |
| | | TCCTTCTCCCAGTCCGGCGATCTGAGGAGACACCAAAGAACACATACCGGCGAAAAGCCTTACAAGTGCCC |
| | | CGAGTGTGGAAAGAGCTTCTCCACCACCGGCGCTCTGACCGAGCACCAGAGAACACACACCGGCGAGAAAC |
| | | CCTATAAATGTCCCGAGTGTGGCAAATCCTTCAGCGACAGCGGCAATCTGAGAGTGCACCAAAGAACCCAT |
| | | ACCGGCGAAAAACCCTACAAATGCCCCGAGTGCGGCAAATCCTTCAGCCAGAGGGCCCATCTGGAGAGGCA |
| | | CCAAAGGACACACACCGGAGAAAAGCCCTACAAGTGTCCCGAGTGTGGAAAAAGCTTTAGCACAAGCGGCG |
| | | AGCTGGTGAGGCATCAAAGGACCCACACCGGCGAAAAGCCCACCGGCAAAAAGACCAGC |
| ZF2 | 39 | CTGGAGCCCGGCGAGAAGCCCTACAAGTGCCCCGAGTGCGGAAAGTCCTTCAGCTCCCCCGCCGATCTGAC |
| (nt) | | AAGACATCAGAGAACCCATACCGGCGAGAAACCTTACAAATGCCCCGAATGTGGCAAGTCCTTTAGCGATC |
| | | CCGGACATCTGGTGAGGCACCAGAGGACACACACCGGCGAAAAGCCCTATAAATGTCCCGAGTGTGGAAAG |
| | | AGCTTTTCTAGAAGCGACAATCTCGTGAGACACCAGAGAACCCACACCGGAGAGAAGCCTTACAAGTGCCC |
| | | CGAGTGCGGCAAATCCTTCAGCCAGAGCTCCTCTCTGGTGAGGCACCAAAGGACCCACACCGGCGAGAAAC |
| | | CTTATAAGTGTCCCGAGTGTGGCAAAAGCTTCAGCACCTCCCACTCTCTGACCGAGCATCAAAGAACCCACA |
| | | CCGGCGAAAAACCTTATAAATGCCCCGAGTGTGGCAAATCCTTCAGCAGAAATGACGCTCTGACAGAGCAC |
| | | CAAAGAACACATACCGGAGAAAAGCCCTACAAATGCCCCGAGTGTGGAAAATCCTTTTCTAGAAACGATGCT |
| | | CTGACCGAACACCAAAGAACACACACCGGCGAAAAGCCTACCGGAAAAAAGACCAGC |
| ZF3 | 40 | CTGGAGCCCGGCGAAAAACCTTACAAGTGCCCCGAGTGCGGAAAGAGCTTCAGCAGAAGCGACAAACTGGT |
| (nt) | | GAGGCATCAAAGGACACATACCGGAGAGAAGCCCTATAAGTGCCCCGAATGTGGCAAATCCTTTTCCCAGAG |
| | | GGCTCATCTGGAAAGACACCAGAGGACCCATACCGGCGAAAAACCCTACAAATGTCCCGAGTGTGGAAAGA |
| | | GCTTTTCCGATCCCGGCCATCTGGTCAGACATCAGAGGACACATACCGGCGAAAAGCCTTACAAGTGTCCCG |
| | | AATGCGGAAAATCCTTCTCCAGAAGCGACAAGCTGGTGAGGCACCAAAGAACCCACACCGGCGAAAAACCC |
| | | TATAAATGCCCCGAGTGCGGCAAGTCCTTTAGCCAGCTGGCCCATCTGAGAGCCCACCAGAGAACACACACC |
| | | GGAGAGAAGCCTTATAAGTGTCCCGAGTGCGGAAAGTCCTTCTCTAGAGCCGACAATCTGACCGAACATCAA |
| | | AGGACACACACCGGCGAGAAACCTTATAAATGCCCCGAGTGCGGAAAAAGCTTTTCCGACTGCAGAGATCTG |
| | | GCTAGACACCAGAGAACCCACACCGGCGAGAAACCCACCGGCAAAAAGACCAGC |
| ZF4 | 41 | CTGGAGCCCGGCGAAAAGCCTTATAAATGTCCCGAATGCGGCAAGAGCTTTAGCCACACCGGCCATCTGCT |
| (nt) | | GGAACACCAAAGGACCCATACCGGCGAAAAGCCCTATAAGTGCCCCGAGTGTGGCAAGAGCTTCAGCACCA |
| | | CCGGCAATCTGACAGTCCATCAGAGGACCCACACCGGAGAGAAACCCTATAAATGCCCCGAGTGTGGAAAG |
| | | TCCTTCTCCGACAAGAAGGATCTGACAAGACACCAGAGGACCCATACCGGCGAGAAACCCTACAAATGCCC |
| | | CGAGTGCGGCAAATCCTTCTCCCAGAGCGGCGATCTGAGGAGACATCAAAGAACACATACCGGCGAAAAAC |
| | | CCTATAAGTGCCCCGAATGCGGCAAGTCCTTCAGCCAGAGGGCCCATCTGGAAAGGCATCAGAGGACACAC |
| | | ACCGGCGAGAAGCCTTACAAATGTCCCGAGTGCGGAAAGAGCTTCTCTAGAAGCGACAAGCTGACCGAGCA |
| | | TCAGAGGACCCACACCGGAGAAAAACCTTACAAGTGCCCCGAGTGCGGCAAAAGCTTCAGCAGAACCGACA |
| | | CACTGAGAGATCACCAAAGGACACACACCGGCGAGAAACCCACCGGCAAAAAGACCAGC |
| ZF5 | 42 | CTGGAGCCCGGCGAGAAGCCTTATAAGTGCCCCGAGTGTGGCAAGAGCTTTAGCCACACCGGCCATCTGCTG |
| (nt) | | GAGCATCAAAGGACACACACCGGAGAAAAGCCCTATAAGTGCCCCGAGTGTGGCAAATCCTTCAGCACCTCC |
| | | GGCAATCTCACCGAACACCAGAGAACACACACCGGAGAAAAACCTTACAAATGTCCCGAGTGTGGAAAGAGC |
| | | TTTTCCACCAGCGGCAATCTGGTGAGACATCAAAGAACACATACCGGCGAAAAACCCTATAAATGCCCCGAG |
| | | TGTGGAAAATCCTTCTCCCAACTGGCCCATCTGAGGGCCCACCAGAGGACACATACCGGAGAAAAACCCTACA |
| | | AATGCCCCGAATGCGGAAAAAGCTTCTCCGAGAGAAGCCATCTGAGAGAGCACCAAAGGACCCATACCGGAG |
| | | AAAAGCCTTACAAGTGTCCCGAGTGCGGAAAAAGCTTTAGCGATCCCGGACATCTGGTGAGACATCAGAGAA |
| | | CCCACACCGGCGAAAAGCCTTATAAATGTCCCGAATGTGGCAAGTCCTTTAGCACCCATCTGGATCTGATTAG |
| | | ACACCAAAGAACCCACACCGGCGAGAAACCCACCGGAAAAAAGACCAGC |
| ZF6 | 43 | CTGGAGCCCGGCGAAAAGCCTTACAAATGTCCCGAGTGCGGAAAGTCCTTCAGCGACCCCGGCGCTCTGGTG |
| (nt) | | AGACATCAAAGAACACATACCGGCGAGAAACCTTATAAATGCCCCGAATGTGGAAAATCCTTCAGCGAAAGA |
| | | AGCCATCTGAGGGAACACCAGAGGACCCACACCGGCGAAAAACCTTATAAGTGCCCCGAATGCGGAAAAAG |
| | | CTTTTCTAGAAGCGATCATCTGACCAACCATCAGAGAACACACACCGGCGAAAAGCCCTATAAATGTCCCGA |
| | | GTGTGGCAAATCCTTTAGCGAGAGGTCCCATCTGAGAGAGCACCAGAGGACACATACCGGAGAGAAGCCCTA |
| | | CAAGTGCCCCGAGTGTGGCAAGAGCTTTAGCAGAAGCGACCATCTGACCAATCATCAAAGGACCCACACCGG |
| | | AGAGAAGCCTTACAAGTGTCCCGAGTGCGGAAAGTCCTTTTCCGATCCCGGCCACCTCGTGAGGCACCAAAG |
| | | AACCCATACCGGCGAGAAACCCTACAAATGCCCCGAGTGTGGAAAGAGCTTCTCCAGAAGCGACAAGCTGGT |
| | | GAGGCATCAGAGGACACACACCGGCGAAAAACCCACCGGCAAGAAAACCAGC |
| ZF7 | 44 | CTGGAGCCCGGAGAGAAGCCCTACAAATGCCCCGAGTGTGGAAAGAGCTTCTCTAGAAATGACGCTCTGAC |
| (nt) | | AGAACACCAGAGGACCCATACCGGCGAGAAACCTTACAAATGCCCCGAGTGCGGAAAAAGCTTTAGCGATT |
| | | GCAGAGATCTGGCTAGACATCAGAGAACACACACCGGCGAGAAGCCCTATAAGTGCCCCGAATGCGGCAA |
| | | GAGCTTTAGCGACCCCGGCCATCTGGTGAGACATCAAAGGACACATACCGGAGAAAAACCTTACAAGTGCC |
| | | CCGAGTGCGGAAAGTCCTTCTCCCAGAGCGGCCATCTCACCGAGCATCAAAGGACCCACACCGGCGAAAAG |
| | | CCTTATAAATGTCCCGAATGTGGCAAGTCCTTCTCTAGAGAGGATAATCTGCACACCCATCAGAGGACCCAC |
| | | ACCGGCGAAAAGCCTTATAAATGCCCCGAATGTGGAAAGTCCTTTTCCACCAAGAACTCTCTGACCGAGCAT |
| | | CAGAGGACACACACCGGAGAGAAACCCTATAAATGTCCCGAGTGTGGCAAGAGCTTCAGCAGAGCCGACAA |
| | | TCTGACAGAGCACCAAAGAACACATACCGGCGAAAAGCCCACCGGCAAAAAGACCAGC |
| ZF8 | 45 | CTGGAGCCCGGCGAGAAACCCTACAAGTGCCCCGAGTGTGGCAAATCCTTCTCTAGATCCGACAAACTGAC |
| (nt) | | CGAACATCAGAGGACCCATACCGGCGAAAAACCTTATAAATGTCCCGAGTGCGGAAAGTCCTTCTCTAGAA |
| | | GGGACGAGCTGAACGTGCATCAGAGAACACATACCGGCGAGAAGCCCTATAAATGCCCCGAATGCGGCAA |
| | | AAGCTTCTCTAGAAGCGATCATCTGACCAACCACCAGAGAACCCATACCGGAGAAAAGCCTTACAAGTGTC |
| | | CCGAATGTGGAAAATCCTTCAGCTCCCCCGCCGATCTGACCAGACACCAAAGGACCCACACCGGCGAGAAG |
| | | CCCTATAAATGCCCCGAGTGCGGCAAGAGCTTTTCCAGATCCGACCATCTGACCAATCATCAAAGAACCCAC |
| | | ACCGGCGAAAAGCCTTATAAATGTCCCGAGTGCGGCAAATCCTTTTCCAGCAAGAAGGCTCTGACCGAGCA |
| | | TCAAAGGACCCATACCGGCGAGAAGCCTTACAAATGCCCCGAGTGTGGAAAGTCCTTTAGCACCCATCTGGA |
| | | TCTGATTAGACACCAGAGGACACACACCGGAGAGAAACCCACCGGCAAAAAGACCAGC |
| ZF9 | 46 | CTGGAGCCCGGCGAGAAACCTTACAAATGCCCCGAGTGCGGCAAGAGCTTCAGCAGAAGCGACGATCTGGT |
| (nt) | | GAGGCACCAAAGAACCCACACCGGCGAAAAACCTTACAAGTGTCCCGAATGCGGAAAGTCCTTCAGCAGAG |
| | | AGGACAATCTGCACACCCACCAGAGAACACACACCGGAGAAAAGCCTTACAAGTGCCCCGAATGCGGCAA |
| | | ATCCTTTTCTAGAAGCGATCATCTGACCACCCACCAAAGAACACATACCGGCGAGAAGCCTTACAAATGTCC |
| | | CGAGTGCGGAAAGTCCTTCTCCCAGAGAGCCAATCTGAGGGCTCATCAAAGGACCCATACCGGCGAAAAGC |
| | | CCTACAAATGCCCCGAGTGCGGAAAATCCTTCAGCCAGCTGGCCCATCTGAGAGCCCACCAAAGGACACAC |
| | | ACCGGAGAGAAACCCTATAAGTGCCCCGAGTGTGGAAAAAGCTTTTCCCAGAGGGCCAATCTGAGGGCCCA |
| | | TCAGAGGACCCATACCGGAGAGAAGCCTTATAAATGTCCCGAGTGOGGAAAAAGCTTCAGCGAGAGGAGCC |
| | | ATCTGAGGGAACATCAAAGAACCCACACCGGCGAAAAACCCACCGGAAAAAAGACCAGC |
| ZF10 | 47 | CTGGAGCCCGGCGAGAAACCCTACAAGTGCCCCGAGTGTGGAAAAAGCTTTAGCCAAAGCGGCGATCTGAGG |
| (nt) | | AGACACCAAAGAACACACACCGGCGAGAAGCCCTACAAATGTCCCGAGTGCGGAAAGAGCTTCAGCCAGAG |
| | | CGGCCATCTGACCGAGCATCAGAGAACCCATACCGGCGAAAAACCTTATAAGTGCCCCGAGTGTGGAAAGTC |
| | | CTTCTCCGAGAGATCCCATCTGAGAGAACACCAGAGGACACACACCGGCGAAAAACCTTATAAGTGTCCCGA |
| | | GTGCGGAAAGTCCTTCAGCGATCCCGGCCATCTGGTGAGACATCAAAGGACACATACCGGCGAAAAACCTTA |
| | | TAAGTGTCCCGAATGCGGCAAGAGCTTTAGCAGAAACGACACACTCACCGAACACCAGAGGACCCACACCGG |
| | | CGAGAAACCCTACAAATGCCCCGAGTGCGGCAAATCCTTTTCTAGAGCCGACAATCTGACCGAACACCAGAG |
| | | GACCCATACCGGAGAAAAGCCTTACAAATGTCCCGAGTGTGGCAAATCCTTCTCCACCCATCTGGATCTGATT |
| | | AGACACCAAAGAACACATACCGGAGAAAAGCCCACCGGAAAAAAGACCAGC |
| ZF11 | 48 | CTGGAGCCCGGCGAAAAACCCTATAAGTGCCCCGAATGTGGAAAGAGCTTCAGCCATACCGGCCATCTGCT |
| (nt) | | GGAACACCAAAGGACACACACCGGCGAGAAACCTTACAAGTGTCCCGAGTGCGGAAAAAGCTTCTCCTCCA |
| | | AAAAGGCTCTCACCGAGCACCAGAGAACACATACCGGCGAAAAGCCTTATAAGTGCCCCGAGTGTGGCAAA |
| | | TCCTTTTCCGACTGTAGAGATCTGGCCAGACATCAAAGAACCCACACCGGAGAGAAACCTTATAAATGCCCC |
| | | GAGTGCGGCAAGTCCTTTAGCCATACCGGCCATCTGCTGGAGCACCAGAGGACCCATACCGGCGAGAAGCC |
| | | TTACAAATGCCCCGAGTGCGGCAAAAGCTTCAGCAGAAATGACGCTCTGACCGAGCATCAAAGGACCCATA |
| | | CCGGCGAAAAGCCCTACAAGTGTCCCGAGTGTGGAAAGTCCTTCTCCCAGAGCGGCGATCTGAGGAGACAC |
| | | CAGAGAACACACACCGGCGAGAAACCCTATAAATGTCCCGAGTGCGGAAAGAGCTTTAGCGACAGCGGCAA |
| | | TCTGAGGGTGCATCAAAGAACACACACCGGCGAAAAACCCACCGGAAAAAAGACAAGC |
| ZF12 | 49 | CACCGGCGAAAAGCCTTATAAGTGCCCCGAGTGCGGCAAGTCCTTCTCTAGAAGCGATCACCTCACCAATCA |
| (nt) | | TCAGAGGACACATACCGGAGAGAAGCCCTATAAGTGCCCCGAGTGCGGCAAGAGCTTTAGCCAGCTGGCTC |
| | | ATCTGAGAGCTCACCAAAGAACCCATACCGGCGAGAAGCCTTACAAATGCCCCGAGTGTGGAAAATCCTTT |
| | | TCCCAGTCCAGCAACCTCGTCAGACATCAAAGGACCCATACCGGCGAAAAGCCTTACAAGTGTCCCGAGTG |
| | | CGGAAAGTCCTTCTCTAGATCCGACAACCTCGTGAGGCACCAGAGAACCCACACCGGCGAGAAACCTTACA |
| | | AATGTCCCGAGTGTGGCAAAAGCTTTTCTAGAAGCGACGAGCTGGTGAGACATCAAAGAACCCATACCGGC |
| | | GAAAAACCTTATAAGTGTCCCGAGTGCGGCAAATCCTTTAGCCAGCTGGCCCATCTGAGGGCCCACCAGAGA |
| | | ACACATACCGGCGAAAAACCCACCGGCAAAAAGACAAGC |
| ZF12 | 115 | CTGGAGCCCGGCGAGAAACCCTATAAATGCCCCGAATGCGGAAAAAGCTTCAGCCAGTCCAGCTCTCTGGTG |
| (nt) | | AGACATCAGAGGACACACACCGGCGAAAAGCCTTATAAGTGCCCCGAGTGCGGCAAGTCCTTCTCTAGAAGC |
| Full | | GATCACCTCACCAATCATCAGAGGACACATACCGGAGAGAAGCCCTATAAGTGCCCCGAGTGCGGCAAGAGC |
| length | | TTTAGCCAGCTGGCTCATCTGAGAGCTCACCAAAGAACCCATACCGGCGAGAAGCCTTACAAATGCCCCGAG |
| | | TGTGGAAAATCCTTTTCCCAGTCCAGCAACCTCGTCAGACATCAAAGGACCCATACCGGCGAAAAGCCTTAC |
| | | AAGTGTCCCGAGTGCGGAAAGTCCTTCTCTAGATCCGACAACCTCGTGAGGCACCAGAGAACCCACACCGGC |
| | | GAGAAACCTTACAAATGTCCCGAGTGTGGCAAAAGCTTTTCTAGAAGCGACGAGCTGGTGAGACATCAAAGA |
| | | ACCCATACCGGCGAAAAACCTTATAAGTGTCCCGAGTGCGGCAAATCCTTTAGCCAGCTGGCCCATCTGAGG |
| | | GCCCACCAGAGAACACATACCGGCGAAAAACCCACCGGCAAAAAGACAAGC |
| ZF9 | 131 | CUGGAGCCCGGCGAGAAACCUUACAAAUGCCCCGAGUGCGGCAAGAGCUUCAGCAGAAGCGACGAUCUGG |
| | | UGAGGCACCAAAGAACCCACACCGGCGAAAAACCUUACAAGUGUCCCGAAUGCGGAAAGUCCUUCAGCAG |
| | | AGAGGACAAUCUGCACACCCACCAGAGAACACACACCGGAGAAAAGCCUUACAAGUGCCCCGAAUGCGGC |
| | | AAAUCCUUUUCUAGAAGCGAUCAUCUGACCACCCACCAAAGAACACAUACCGGCGAGAAGCCUUACAAAU |
| | | GUCCCGAGUGCGGAAAGUCCUUCUCCCAGAGAGCCAAUCUGAGGGCUCAUCAAAGGACCCAUACCGGCGA |
| | | AAAGCCCUACAAAUGCCCCGAGUGCGGAAAAUCCUUCAGCCAGCUGGCCCAUCUGAGAGCCCACCAAAGG |
| | | ACACACACCGGAGAGAAACCCUAUAAGUGCCCCGAGUGUGGAAAAAGCUUUUCCCAGAGGGCCAAUCUGA |
| | | GGGCCCAUCAGAGGACCCAUACCGGAGAGAAGCCUUAUAAAUGUCCCGAGUGCGGAAAAAGCUUCAGCGA |
| | | GAGGAGCCAUCUGAGGGAACAUCAAAGAACCCACACCGGCGAAAAACCCACCGGAAAAAAGACCAGC |
| ZF54 | 173 | CTGGAGCCTGGAGAGAAACCCTACAAATGCCCGGAATGCGGGAAGTCCTTTTCCGAACGCTCGCACCTGAGG |
| | | GAACACCAGAGAACTCACACCGGCGAAAAACCCTATAAGTGCCCAGAATGCGGAAAGAGCTTTTCACGGTCG |
| | | GACAACCTCGTGCGGCACCAACGCACTCATACCGGAGAGAAGCCGTACAAGTGTCCTGAGTGCGGAAAGTCA |
| | | TTCTCCGACTGCCGGGATTTGGCCCGCCACCAAAGAACACACACTGGCGAAAAGCCCTACAAGTGCCCGGAG |
| | | TGTGGAAAGTCCTTCAGCACTTCCGGAGAGCTGGTCCGGCACCAGAGGACCCACACCGGGGAGAAGCCTTAC |
| | | AAATGTCCAGAGTGCGGTAAAAGCTTCTCCACCACCGGCAACCTCACCGTGCACCAGCGGACCCACACTGGA |
| | | GAAAAGCCGTATAAATGCCCCGAATGCGGCAAGAGCTTCTCGCGATCCGATAAGCTTGTGCGGCATCAGAGA |
| | | ACGCACACTGGGGAAAAGCCTTATAAGTGTCCGGAGTGCGGCAAATCCTTCTCCCGCACTGACACCCTGCGG |
| | | GACCATCAGCGCACCCATACCGGCAAAAAGACCTCT |
| ZF61 | 174 | CTTGAACCCGGGGAGAAGCCCTACAAGTGCCCGGAATGCGGAAAGAGCTTCAGCCAGAAGTCCTCGCTGATC |
| | | GCGCACCAGAGGACTCACACCGGCGAAAAGCCATACAAGTGTCCTGAGTGTGGCAAATCCTTCTCGCACAAG |
| | | AACGCACTGCAGAACCACCAGAGAACCCACACCGGGGAAAAGCCGTATAAGTGCCCCGAATGTGGAAAGTC |
| | | GTTCAGCCAGTCATCCAACCTCGTGCGCCATCAAAGGACTCATACCGGAGAGAAACCTTACAAATGCCCTGA |
| | | ATGCGGCAAATCTTTCTCCCGGAATGATGCCCTGACCGAGCACCAGCGCACTCACACGGGAGAGAAGCCGTA |
| | | CAAATGTCCGGAGTGCGGAAAGTCCTTCTCCGACAAGAAGGACTTGACCAGACACCAGCGGACCCATACTGG |
| | | CGAAAAACCCTATAAGTGTCCAGAGTGCGGGAAGTCCTTTAGCCAAGCCGGTCACCTCGCTTCCCACCAACG |
| | | GACCCACACAGGAGAAAAGCCTTATAAATGCCCCGAGTGCGGCAAAAGCTTCTCCGATAAGAAGGACCTGAC |
| | | TCGGCATCAGCGCACCCATACCGGAAAGAAAACCTCA |
| ZF67 | 175 | CTGGAGCCTGGCGAAAAACCCTATAAGTGCCCAGAATGCGGAAAGAGCTTTTCACGGTCGGACAACCTCGTG |
| | | CGGCACCAACGCACTCATACCGGAGAGAAGCCGTACAAGTGTCCTGAGTGCGGAAAGTCATTCTCCGACTGC |
| | | CGGGATTTGGCCCGCCACCAAAGAACACACACTGGCGAAAAGCCCTACAAGTGCCCGGAGTGTGGAAAGTCC |
| | | TTCAGCACTTCCGGAGAGCTGGTCCGGCACCAGAGGACCCACACCGGGGAGAAGCCTTACAAATGTCCAGAG |
| | | TGCGGTAAAAGCTTCTCCACCACCGGCAACCTCACCGTGCACCAGCGGACCCACACTGGAGAAAAGCCGTAT |
| | | AAATGCCCCGAATGCGGCAAGAGCTTCTCGCGATCCGATAAGCTTGTGCGGCATCAGAGAACGCACACTGGG |
| | | GAAAAGCCTTATAAGTGTCCGGAGTGCGGCAAATCCTTCTCCCGCACTGACACCCTGCGGGACCACCAGAGA |
| | | ACCCATACTGGCGAGAAGCCATACAAATGCCCGGAATGTGGAAAGAGTTTCTCGCGCGAGGACAACCTCCAC |
| | | ACCCATCAGCGCACCCATACCGGCAAAAAGACCTCT |
| ZF68 | 176 | CTGGAACCCGGAGAGAAACCCTACAAATGCCCAGAGTGCGGCAAATCGTTCTCACGGTCCGATCACCTCACC |
| | | ACCCACCAGAGGACCCATACCGGGGAGAAGCCTTACAAGTGTCCTGAGTGTGGAAAGTCCTTCAGCCAAAAG |
| | | TCCTCGCTGATCGCACACCAGCGCACGCACACTGGGGAAAAGCCATATAAATGCCCGGAGTGTGGCAAATCC |
| | | TTCTCCCGCCGCGACGAACTGAACGTGCACCAGAGAACCCACACTGGAGAGAAGCCGTATAAGTGTCCGGAG |
| | | TGCGGAAAGAGCTTCTCGCAATCCGGGGACCTTCGGAGACATCAGAGGACACACACTGGCGAAAAGCCCTAT |
| | | AAGTGCCCTGAGTGCGGGAAGTCCTTTAGCCAAGCCGGTCACCTGGCCTCCCACCAACGGACTCACACCGGC |
| | | GAAAAACCGTACAAGTGCCCCGAATGCGGAAAGTCGTTCTCTACCTCCGGAAACTTGACCGAACACCAGCGG |
| | | ACCCACACCGGAGAAAAGCCGTACAAATGTCCTGAATGCGGCAAAAGCTTCAGCCAGGCCGGTCATCTCGCG |
| | | AGCCATCAGCGGACTCATACTGGCAAAAAGACCTCA |

In some embodiments, an expression repression comprises a targeting moiety comprising an engineered DNA binding domain (DBD), e.g., a Zn finger domain comprising a Zn finger (ZFN) that binds to a target sequence, e.g., a promoter or transcription start site (TSS)) sequence operably linked to a target gene (e.g., MYC), e.g., a sequence proximal to the transcription regulatory element, e.g., an anchor sequence of an anchor sequence mediated conjunction (ASMC) comprising a target gene (e.g., MYC), e.g., a sequence proximal to the anchor sequence in mouse genome. In some embodiments, the ZFN can be engineered to carry epigenetic effector molecules to target sites. In some embodiments, the targeting moiety comprises a Zn Finger domain that comprises 2, 3, 4, 5, 6, 7, or 8 zinc fingers. The amino acid sequences of exemplary targeting moieties disclosed herein are listed in Table 14. The nucleotide sequences encoding exemplary targeting moieties disclosed herein are listed in Table 15. In some embodiments, an expression repressor or system described herein comprises a targeting moiety having a sequence set forth in Table 14, or a sequence with at least 70%, 75%, 80%, 85%, 90%, 95%, 98%, or 99% identity thereto. In some embodiments, a nucleic acid described herein comprises a sequence set forth in Table 15, or a sequence with at least 70%, 75%, 80%, 85%, 90%, 95%, 98%, or 99% identity thereto.

TABLE 14
Amino acid sequences of exemplary mouse-specific targeting molettes

| | SEQ | |
|---|---|---|
| | ID | |
| Name | NO. | SEQUENCE |
| ZF15 | 154 | LEPGEKPYKCPECGKSFSTSGELVRHQRTHTGEKPYKCPECGKSFSERSHLREHQRTHTGEKPYKCPECGK |
| (aa) | | SFSRNDTLTEHQRTHTGEKPYKCPECGKSFSRADNLTEHQRTHTGEKPYKCPECGKSFSTSGSLVRHQRTH |
| | | TGEKPYKCPECGKSFSSPADLTRHQRTHTGEKPYKCPECGKSFSDSGNLRVHQRTHTGEKPTGKKTS |
| ZF16 | 155 | LEPGEKPYKCPECGKSFSRSDKLVRHQRTHTGEKPYKCPECGKSFSQRANLRAHQRTHTGEKPYKCPECG |
| (aa) | | KSFSRADNLTEHQRTHTGEKPYKCPECGKSFSQSSSLVRHQRTHTGEKPYKCPECGKSFSDKKDLTRHQRT |
| | | HTGEKPYKCPECGKSFSSPADLTRHQRTHTGEKPYKCPECGKSFSQSGHLTEHQRTHTGEKPTGKKTS |
| ZF17 | 156 | LEPGEKPYKCPECGKSFSQSGDLRRHQRTHTGEKPYKCPECGKSFSQSGDLRRHQRTHTGEKPYKCPECG |
| (aa) | | KSFSERSHLREHQRTHTGEKPYKCPECGKSFSTTGNLTVHQRTHTGEKPYKCPECGKSFSHRTTLTNHQRT |
| | | HTGEKPYKCPECGKSFSDSGNLRVHQRTHTGEKPYKCPECGKSFSQSSSLVRHQRTHTGEKPTGKKTS |

TABLE 15
Nucleotide sequences of exemplary mouse-specific targeting moieties

| | SEQ | |
|---|---|---|
| | ID | |
| Name | NO. | SEQUENCE |
| ZF15 | 157 | CTTGAGCCCGGAGAAAAGCCATACAAATGTCCTGAATGCGGAAAGTCATTTTCTACGAGCGGCGAAC |
| nt | | TCGTGCGGCACCAAAGGACTCATACCGGCGAAAAGCCTTACAAATGCCCGGAGTGCGGAAAAAGCTT |
| | | CTCCGAGCGCTCGCACTTGCGGGAACACCAGCGAACCCACACAGGGGAGAAACCGTATAAGTGCCCA |
| | | GAGTGCGGCAAATCGTTCTCCCGGAACGACACCCTGACCGAACACCAACGCACTCATACTGGCGAAA |
| | | AACCTTACAAGTGCCCTGAGTGTGGAAAGAGCTTCTCCCGCGCCGATAACCTGACCGAGCACCAGCG |
| | | GACCCATACCGGGGAAAAGCCGTACAAGTGTCCGGAATGCGGCAAAAGCTTCAGCACCTCGGGTTCC |
| | | CTGGTCCGGCATCAGAGAACTCACACCGGAGAGAAACCCTATAAGTGTCCTGAGTGCGGGAAGTCCT |
| | | TTTCATCGCCCGCGGACCTGACTAGACACCAGAGGACCCACACCGGGGAGAAGCCCTACAAGTGCCC |
| | | CGAATGTGGAAAGTCCTTCTCCGACTCCGGCAACCTCCGGGTGCACCAGCGCACCCACACTGGAGAG |
| | | AAGCCGACCGGAAAGAAAACTTCC |
| ZF16 | 158 | CTGGAACCCGGAGAAAAACCTTATAAGTGCCCTGAATGCGGAAAGTCATTCTCGAGGTCGGACAAGC |
| nt | | TCGTGCGGCACCAGAGGACACACACCGGGGAAAAGCCATACAAGTGTCCCGAATGTGGAAAGTCCTT |
| | | CAGCCAACGCGCCAACCTGAGAGCTCATCAGCGGACTCACACTGGCGAAAAACCGTACAAATGCCCC |
| | | GAATGCGGCAAAAGCTTCTCCCGCGCCGACAACTTGACCGAGCACCAGCGGACCCATACCGGCGAAA |
| | | AGCCGTACAAGTGCCCGGAGTGTGGGAAGTCGTTCAGCCAGTCCTCTTCCCTCGTGCGCCACCAACGC |
| | | ACCCATACTGGGGAGAAGCCCTATAAGTGTCCTGAGTGTGGCAAATCATTCAGCGATAAGAAGGATC |
| | | TTACCCGGCACCAACGGACTCATACCGGAGAGAAGCCTTACAAGTGCCCCGAGTGCGGAAAGAGCTT |
| | | CTCGTCCCCGGCGGACCTGACTAGACACCAGCGCACCCACACCGGAGAAAAGCCCTACAAGTGCCCA |
| | | GAGTGCGGGAAGTCCTTTTCCCAATCCGGTCACCTGACTGAGCACCAGAGAACCCACACGGGAGAGA |
| | | AACCGACCGGAAAGAAAACCTCC |
| ZF17 | 159 | TTGGAACCCGGAGAAAAGCCATACAAATGCCCCGAATGCGGAAAGTCGTTCAGCCAGTCCGGCGACC |
| nt | | TCAGACGGCACCAACGGACTCACACCGGCGAAAAACCGTACAAGTGCCCAGAGTGCGGCAAAAGCTT |
| | | TAGCCAGTCGGGCGATCTGCGGAGACATCAGCGCACTCACACTGGTGAAAAGCCCTACAAGTGTCCT |
| | | GAGTGCGGGAAGTCCTTCAGCGAGCGCTCCCATCTTCGCGAGCACCAGAGAACCCACACTGGAGAAA |
| | | AACCTTATAAGTGCCCTGAGTGTGGCAAATCCTTCTCAACCACCGGCAACCTGACTGTGCACCAGCGG |
| | | ACCCACACAGGGGAGAAGCCTTACAAGTGCCCGGAGTGTGGGAAGTCATTCTCCCATCGGACGACCC |
| | | TGACCAACCACCAGAGGACCCATACTGGCGAAAAGCCGTATAAGTGTCCGGAGTGCGGAAAGAGCTT |
| | | CTCCGACTCCGGAAACCTCAGGGTGCACCAACGCACCCACACCGGAGAGAAGCCGTACAAATGTCCC |
| | | GAATGTGGAAAGTCCTTCTCCCAATCCTCTTCGCTGGTCCGGCACCAGCGAACTCATACCGGGGAAAA |
| | | GCCCACCGGAAAGAAAACCTCG |

### Nucleic Acid Molecule

In some embodiments, a targeting moiety is or comprises a DNA-binding domain from a nuclease. For example, the recognition sequences of homing endonucleases and meganucleases such as I-SceI, I-CeuI, PI-PspI, PI-Sce, I-SceIV, I-CsmI, I-PanI, I-SceII, I-Ppol, I-SceIII, I-CreI, I-TevI, I-TevII and I-TevIII are known. See also U.S. Pat. Nos. 5,420,032; 6,833,252; Belfort, et al. (1997) Nucleic Acids Res. 25:3379-3388; Dujon, et al. (1989) Gene 82:115-118; Perler, et al. (1994) Nucleic Acids Res. 22:1125-1127; Jasin (1996) Trends Genet. 12:224-228; Gimble, et al. (1996); J. Mol. Biol. 263:163-180; Argast, et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier, et al. (2002) Molec. Cell 10:895-905; Epinat, et al. (2003) Nucleic Acids Res. 31:2952-2962; Ashworth, et al. (2006) Nature 441:656-659; Paques, et al. (2007) Current Gene Therapy 7:49-66; U.S. Patent Publication No. 2007/0117128.

In some embodiments, a DNA-targeting moiety comprises or is nucleic acid. In some embodiments, a nucleic acid that may be included in a DNA-targeting moiety, may be or comprise DNA, RNA, and/or an artificial or synthetic nucleic acid or nucleic acid analog or mimic. For example, in some embodiments, a nucleic acid may be or include one or more of genomic DNA (gDNA), complementary DNA (cDNA), a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with a targeted genomic complex as described herein), etc. In some embodiments, a nucleic acid may include one or more residues that is not a naturally-occurring DNA or RNA residue, may include one or more linkages that is/are not phosphodiester bonds (e.g., that may be, for example, phosphorothioate bonds, etc.), and/or may include one or more modifications such as, for example, a 2′O modification such as 2′-OmeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

A nucleic acid suitable for use in an expression repressor, e.g., in the DNA-targeting moiety, may include, but is not limited to, DNA, RNA, modified oligonucleotides (e.g., chemical modifications, such as modifications that alter backbone linkages, sugar molecules, and/or nucleic acid bases), and artificial nucleic acids. In some embodiments, a nucleic acid includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules.

In some embodiments, a DNA-targeting moiety comprises a nucleic acid with a length from about 15-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 215-190, 20-190, 30-190, 40-190, 50-190, 60-190, 70-190, 80-190, 90-190, 100-190, 110-190, 120-190, 130-190, 140-190, 150-190, 160-190, 170-190, 180-190, 15-180, 20-180, 30-180, 40-180, 50-180, 60-180, 70-180, 80-180, 90-180, 100-180, 110-180, 120-180, 130-180, 140-180, 150-180, 160-180, 170-180, 15-170, 20-170, 30-170, 40-170, 50-170, 60-170, 70-170, 80-170, 90-170, 100-170, 110-170, 120-170, 130-170, 140-170, 150-170, 160-170, 15-160, 20-160, 30-160, 40-160, 50-160, 60-160, 70-160, 80-160, 90-160, 100-160, 110-160, 120-160, 130-160, 140-160, 150-160, 215-150, 20-150, 30-150, 40-150, 50-150, 60-150, 70-150, 80-150, 90-150, 100-150, 110-150, 120-150, 130-150, 140-150, 15-140, 20-140, 30-140, 40-140, 50-140, 60-140, 70-140, 80-140, 90-140, 100-140, 110-140, 120-140, 130-140, 15-130, 20-130, 30-130, 40-130, 50-130, 60-130, 70-130, 80-130, 90-130, 100-130, 110-130, 120-130, 215-120, 20-120, 30-120, 40-120, 50-120, 60-120, 70-120, 80-120, 90-120, 100-120, 110-120, 15-110, 20-110, 30-110, 40-110, 50-110, 60-110, 70-110, 80-110, 90-110, 100-110, 15-100, 20-100, 30-100, 40-100, 50-100, 60-100, 70-100, 80-100, 90-100, 15-90, 20-90, 30-90, 40-90, 50-90, 60-90, 70-90, 80-90, 15-80, 20-80, 30-80, 40-80, 50-80, 60-80, 70-80, 15-70, 20-70, 30-70, 40-70, 50-70, 60-70, 15-60, 20-60, 30-60, 40-60, 50-60, 15-50, 20-50, 30-50, 40-50, 15-40, 20-40, 30-40, 15-30, 20-30, or 15-20 nucleotides, or any range therebetween.

Effector Moieties

In some embodiments, expression repressors of the present disclosure comprise one or more effector moieties. In some embodiments, an effector moiety, when used as part of an expressor repressor or an expression repression system described herein, decreases expression of a target gene in a cell.

In some embodiments, the effector moiety has functionality unrelated to the binding of the targeting moiety. For example, effector moieties may target, e.g., bind, a genomic sequence element or genomic complex component proximal to the genomic sequence element targeted by the targeting moiety or recruit a transcription factor. As a further example, an effector moiety may comprise an enzymatic activity, e.g., a genetic modification functionality.

In some embodiments, an effector moiety comprises an epigenetic modifying moiety. In some embodiments, an effector moiety comprises a DNA modifying functionality, e.g., a DNA methyltransferase. In some embodiments, an effector moiety is or comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.

In some embodiments, an effector moiety comprises a transcription repressor. In some embodiments the transcription repressor blocks recruitment of a factor that stimulates or promotes transcription, e.g., of the target gene. In some embodiments, the transcription repressor recruits a factor that inhibits transcription, e.g., of the target gene. In some embodiments, an effector moiety, e.g., transcription repressor, is or comprises a protein chosen from KRAB, MeCP2, HP1, RBBP4, REST, FOG1, SUZ12, or a functional variant or fragment of any thereof.

In some embodiments an effector moiety promotes epigenetic modification, e.g., directly or indirectly. For example, an effector moiety can indirectly promote epigenetic modification by recruiting an endogenous protein that epigenetically modifies the chromatin. An effector moiety can directly promote epigenetic modification by catalyzing epigenetic modification, wherein the effector moiety comprises enzymatic activity and directly places an epigenetic mark on the chromatin.

In some embodiments, an effector moiety comprises a histone modifying functionality, e.g., a histone methyltransferase, histone demethylase, or histone deacetylase activity. In some embodiments, a effector moiety is or comprises a protein chosen from KDM1A (i.e., LSD1), KDM1B (i.e., LSD2), KDM2A, KDM2B, KDM5A, KDM5B, KDM5C, KDM5D, KDM4B, NO66, or a functional variant or fragment of any thereof. In some embodiments, a effector moiety is or comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.

In some embodiments, an effector moiety comprises a protein having a functionality described herein. In some embodiments, an effector moiety is or comprises a protein selected from: KRAB (e.g., as according to NP_056209.2 or the protein encoded by NM_015394.5); a SET domain (e.g., the SET domain of: SETDB1 (e.g., as according to NP_001353347.1 or the protein encoded by NM_001366418.1); EZH2 (e.g., as according to NP-004447.2 or the protein encoded by NM_004456.5); G9A (e.g., as according to NP_001350618.1 or the protein encoded by NM_001363689.1); or SUV39H1 (e.g., as according to NP_003164.1 or the protein encoded by NIVI_003173.4)); histonc demethylase LSD1 (e.g., as according to NP_055828.2 or the protein encoded by NM_015013.4); FOG1 (e.g., the N-terminal residues of FOG1) (e.g., as according to NP_722520.2 or the protein encoded by NM_153813.3); or KAP1 (e.g., as according to NP_005753.1 or the protein encoded by NM_005762.3); a functional fragment or variant of any thereof, or a polypeptide with a sequence that has at least 80, 85, 90, 91, 92, 93, 94, 95, 96, 97, 98, or 99% identity to any of the above-referenced sequences.

In some embodiments, a effector moiety is or comprises a protein selected from: DNMT3A (e.g., human DNMT3A) (e.g., as according to NP_072046.2 or the protein encoded by NM_022552.4); DNMT3B (e.g., as according to NP_008823.1 or the protein encoded by NM_006892.4); DNMT3L (e.g., as according to NP_787063.1 or the protein encoded by NM_175867.3); DNMT3A/3L complex, bacterial MQ1 (e.g., as according to CAA35058.1 or P15840.3); a functional fragment of any thereof, or a polypeptide with a sequence that has at least 80, 85, 90, 91, 92, 93, 94, 95, 96, 97, 98, or 99% identity to any of the above-referenced sequences.

In another aspect, the disclosure is directed to an expression repressor or a polypeptide comprising one or more (e.g., one) targeting moiety and one or more effector moiety, wherein the one or more effector moiety is or comprises Krueppel-associated box (KRAB) e.g., as according to NP_056209.2 or the protein encoded by NM_015394.5 or a functional variant or fragment thereof. In some embodiments, KRAB is a synthetic KRAB construct. In some embodiments, KRAB comprises an amino acid sequence of SEQ ID NO: 18:

(SEQ ID NO: 18)
DAKSLTAWSRTLVTFKDVFVDFTREEWKLLDTAQQILYRNVMLENYKNLV
SLGYQLTKPDVILRLEKGEEPWLVEREIHQETHPDSETAFEIKSSV

In some embodiments, the KRAB effector moiety is encoded by a nucleotide sequence of SEQ ID NO: 51. In some embodiments, a nucleotide sequence described herein comprises a sequence of SEQ ID NO: 51 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 51)
GACGCCAAGAGCCTGACCGCCTGGAGCCGGACCCTGGTGACCTTCAAGGA
CGTGTTCGTGGACTTCACCCGGGAGGAGTGGAAGCTGCTGGACACCGCCC
AGCAGATCCTGTACCGGAACGTGATGCTGGAGAACTACAAGAACCTGGTG
AGCCTGGGCTACCAGCTGACCAAGCCCGACGTGATCCTGCGGCTGGAGAA
GGGCGAGGAGCCCTGGCTGGTGGAGCGGGAGATCCACCAGGAGACCCACC
CCGACAGCGAGACCGCCTTCGAGATCAAGAGCAGCGTG

In some embodiments, KRAB for use in a polypeptide or an expression repressor described herein is a variant, e.g., comprising one or more mutations, relative to the KRAB sequence of SEQ ID NO: 18. In some embodiments, an KRAB variant comprises one or more amino acid substitutions, deletions, or insertions relative to SEQ ID NO: 18.

In some embodiments, the polypeptide or the expression repressor is a fusion protein comprising a effector moiety that is or comprises KRAB and a DNA-targeting moiety. In some embodiments, the targeting moiety is or comprises a zinc finger domain, TAL domain, or CRISPR/Cas domain, e.g., comprising a CRISPR/Cas protein, e.g., a dCas9 protein. In some embodiments, the polypeptide or the expression repressor comprises an additional moiety described herein. In some embodiments, the polypeptide or the expression repressor decreases expression of a target gene, e.g., MYC. In some embodiments, the polypeptide or the expression repressor may be used in methods of modulating, e.g., decreasing, gene expression, methods of treating a condition, or methods of epigenetically modifying a target gene, e.g., MYC or transcription control element described herein, e.g., in place of an expression repression system. In some embodiments, an expression repression system comprises two or more (e.g., two, three, or four) expression repressors, wherein the first expression repressor comprises an effector moiety comprising the KRAB sequence of SEQ ID NO: 18, or a functional variant or fragment thereof.

In another aspect, the disclosure is directed to a expression repressor or a polypeptide comprising one or more (e.g., one) targeting moiety and one or more effector moiety, wherein the one or more effector moiety is or comprises MQ1, e.g., bacterial MQ1, or a functional variant or fragment thereof. In some embodiments, MQ1 is Mollicutes spiroplasma MQ1. In some embodiments, MQ1 is Spiroplasma monobiae MQ1. In some embodiments, MQ1 is MQ1 from strain ATCC 33825 and/or corresponding to Uniprot ID P15840. In some embodiments, MQ1 comprises an amino acid sequence of SEQ ID NO: 19. In some embodiments, MQ1 comprises an amino acid sequence of SEQ ID NO: 87. In some embodiments, an effector domain described herein comprises SEQ ID NO: 19 or 87, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 19)
SKVENKTKKLRVFEAFAGIGAQRKALEKVRKDEYEIVGLAEWYVPAIVMY
QAIHNNFHTKLEYKSVSREEMIDYLENKTLSWNSKNPVSNGYWKRKKDDE
LKIIYNAIKLSEKEGNIFDIRDLYKRTLKNIDLLTYSFPCQDLSQQGIQK
GMKRGSGTRSGLLWEIERALDSTEKNDLPKYLLMENVGALLHKKNEEELN
QWKQKLESLGYQNSIEVLNAADFGSSQARRRVFMISTLNEFVELPKGDKK
PKSIKKVLNKIVSEKDILNNLLKYNLTEFKKTKSNINKASLIGYSKFNSE
GYVYDPEFTGPTLTASGANSRIKIKDGSNIRKMNSDETFLYIGFDSQDGK
RVNEIEFLTENQKIFVCGNSISVEVLEAIIDKIGG
(SEQ ID NO: 87)
MSKVENKTKKLRVFEAFAGIGAQRKALEKVRKDEYEIVGLAEWYVPAIVM
YQAIHNNFHTKLEYKSVSREEMIDYLENKTLSWNSKNPVSNGYWKRKKDD
ELKIIYNAIKLSEKEGNIFDIRDLYKRTLKNIDLLTYSFPCQDLSQQGIQ
KGMKRGSGTRSGLLWEIERALDSTEKNDLPKYLLMENVGALLHKKNEEEL
NQWKQKLESLGYQNSIEVLNAADFGSSQARRRVFMISTLNEFVELPKGDK
KPKSIKKVLNKIVSEKDILNNLLKYNLTEFKKTKSNINKASLIGYSKFNS
EGYVYDPEFTGPTLTASGANSRIKIKDGSNIRKMNSDETFLYIGFDSQDG
KRVNEIEFLTENQKIFVCGNSISVEVLEAIIDKIGG

In some embodiments, MQ1 is encoded by a nucleotide sequence of SEQ ID NO: 52 or 132. In some embodiments, a nucleic acid described herein comprises a sequence of SEQ ID NO: 52, 132 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 52)
AGCAAGGTGGAGAACAAGACCAAGAAGCTGCGGGTGTTCGAGGCCTTCGC
CGGCATCGGCGCCCAGCGGAAGGCCCTGGAGAAGGTGCGGAAGGACGAGT
ACGAGATCGTGGGCCTGGCCGAGTGGTACGTGCCCGCCATCGTGATGTAC
CAGGCCATCCACAACAACTTCCACACCAAGCTGGAGTACAAGAGCGTGAG
CCGGGAGGAGATGATCGACTACCTGGAGAACAAGACCCTGAGCTGGAACA
GCAAGAACCCCGTGAGCAACGGCTACTGGAAGCGGAAGAAGGACGACGAG
CTGAAGATCATCTACAACGCCATCAAGCTGAGCGAGAAGGAGGGCAACAT
CTTCGACATCCGGGACCTGTACAAGCGGACCCTGAAGAACATCGACCTGC
TGACCTACAGCTTCCCCTGCCAGGACCTGAGCCAGCAGGGCATCCAGAAG
GGCATGAAGCGGGGCAGCGGCACCCGGAGCGGCCTGCTGTGGGAGATCGA
GCGGGCCCTGGACAGCACCGAGAAGAACGACCTGCCCAAGTACCTGCTGA
TGGAGAACGTGGGCGCCCTGCTGCACAAGAAGAACGAGGAGGAGCTGAAC
CAGTGGAAGCAGAAGCTGGAGAGCCTGGGCTACCAGAACAGCATCGAGGT
GCTGAACGCCGCCGACTTCGGCAGCAGCCAGGCCCGGCGGCGGGTGTTCA
TGATCAGCACCCTGAACGAGTTCGTGGAGCTGCCCAAGGGCGACAAGAAG
CCCAAGAGCATCAAGAAGGTGCTGAACAAGATCGTGAGCGAGAAGGACAT
CCTGAACAACCTGCTGAAGTACAACCTGACCGAGTTCAAGAAAACCAAGA
GCAACATCAACAAGGCCAGCCTGATCGGCTACAGCAAGTTCAACAGCGAG
GGCTACGTGTACGACCCCGAGTTCACCGGCCCCACCCTGACCGCCAGCGG
CGCCAACAGCCGGATCAAGATCAAGGACGGCAGCAACATCCGGAAGATGA
ACAGCGACGAGACCTTCCTGTACATCGGCTTCGACAGCCAGGACGGCAAG
CGGGTGAACGAGATCGAGTTCCTGACCGAGAACCAGAAGATCTTCGTGTG
CGGCAACAGCATCAGCGTGGAGGTGCTGGAGGCCATCATCGACAAGATCG
GCGGC
(SEQ ID NO: 132)
AGCAAGGUGGAGAACAAGACCAAGAAGCUGCGGGUGUUCGAGGCCUUCGC
CGGCAUCGGCGCCCAGCGGAAGGCCCUGGAGAAGGUGCGGAAGGACGAGU
ACGAGAUCGUGGGCCUGGCCGAGUGGUACGUGCCCGCCAUCGUGAUGUAC
CAGGCCAUCCACAACAACUUCCACACCAAGCUGGAGUACAAGAGCGUGAG
CCGGGAGGAGAUGAUCGACUACCUGGAGAACAAGACCCUGAGCUGGAACA
GCAAGAACCCCGUGAGCAACGGCUACUGGAAGCGGAAGAAGGACGACGAG
CUGAAGAUCAUCUACAACGCCAUCAAGCUGAGCGAGAAGGAGGGCAACAU
CUUCGACAUCCGGGACCUGUACAAGCGGACCCUGAAGAACAUCGACCUGC
UGACCUACAGCUUCCCCUGCCAGGACCUGAGCCAGCAGGGCAUCCAGAAG
GGCAUGAAGCGGGGCAGCGGCACCCGGAGCGGCCUGCUGUGGGAGAUCGA
GCGGGCCCUGGACAGCACCGAGAAGAACGACCUGCCCAAGUACCUGCUGA
UGGAGAACGUGGGCGCCCUGCUGCACAAGAAGAACGAGGAGGAGCUGAAC
CAGUGGAAGCAGAAGCUGGAGAGCCUGGGCUACCAGAACAGCAUCGAGGU
GCUGAACGCCGCCGACUUCGGCAGCAGCCAGGCCCGGCGGCGGGUGUUCA
UGAUCAGCACCCUGAACGAGUUCGUGGAGCUGCCCAAGGGCGACAAGAAG
CCCAAGAGCAUCAAGAAGGUGCUGAACAAGAUCGUGAGCGAGAAGGACAU
CCUGAACAACCUGCUGAAGUACAACCUGACCGAGUUCAAGAAaACCAAGA
GCAACAUCAACAAGGCCAGCCUGAUCGGCUACAGCAAGUUCAACAGCGAG
GGCUACGUGUACGACCCCGAGUUCACCGGCCCCACCCUGACCGCCAGCGG
CGCCAACAGCCGGAUCAAGAUCAAGGACGGCAGCAACAUCCGGAAGAUGA
ACAGCGACGAGACCUUCCUGUACAUCGGCUUCGACAGCCAGGACGGCAAG
CGGGUGAACGAGAUCGAGUUCCUGACCGAGAACCAGAAGAUCUUCGUGUG
CGGCAACAGCAUCAGCGUGGAGGUGCUGGAGGCCAUCAUCGACAAGAUCG
GCGGC

In some embodiments, MQ1 for use in a polypeptide or an expression repressor described herein is a variant, e.g., comprising one or more mutations, relative to a wildtype MQ1 (e.g., SEQ ID NO: 19). In some embodiments, an MQ1 variant comprises one or more amino acid substitutions, deletions, or insertions relative to a wildtype MQ1, e.g., the MQ1 of SEQ ID NO: 19. In some embodiments, an MQ1 variant comprises a K297P substitution. In some embodiments, an MQ1 variant comprises a N299C substitution. In some embodiments, an MQ1 variant comprises a E301Y substitution. In some embodiments, an MQ1 variant comprises a Q147L substitution (e.g., and has reduced DNA methyltransferase activity relative to wildtype MQ1). In some embodiments, an MQ1 variant comprises K297P, N299C, and E301Y substitutions (e.g., and has reduced DNA binding affinity relative to wildtype MQ1). In some embodiments, an MQ1 variant comprises Q147L, K297P, N299C, and E301Y substitutions (e.g., and has reduced DNA methyltransferase activity and DNA binding affinity relative to wildtype MQ1).

In some embodiments, the polypeptide or the expression repressor is a fusion protein comprising an effector moiety that is or comprises MQ1 and a targeting moiety is or comprises a zinc finger domain, TAL domain, or CRISPR/Cas domain, a dCas9 domain. In some embodiments, the polypeptide or the expression repressor comprises an additional moiety described herein. In some embodiments, the polypeptide or the expression repressor decreases expression of a target gene, e.g., MYC. In some embodiments, the polypeptide or the expression repressor may be used in methods of modulating, e.g., decreasing, gene expression, methods of treating a condition, or methods of epigenetically modifying a target gene, e.g., MYC or transcription control element described herein, e.g., in place of an expression repression system. In some embodiments, an expression repression system comprises two or more (e.g., two, three, or four) expression repressors, wherein the first expression repressor comprises an effector moiety comprising MQ1, e.g., bacterial MQ1, or a functional variant or fragment thereof.

In another aspect, the disclosure is directed to an expression repressor or a polypeptide comprising one or more (e.g., one) targeting moiety and one or more effector moiety, wherein the one or more effector moiety is or comprises DNMT1, e.g., human DNMT1, or a functional variant or fragment thereof. In some embodiments, DNMT1 is human DNMT1, e.g., corresponding to Gene ID 1786, e.g., corresponding to UniProt ID P26358.2. In some embodiments, DNMT1 comprises an amino acid sequence of SEQ ID NO: 20. In some embodiments, an effector domain described herein comprises a sequence according to SEQ ID NO: 20 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto:

(SEQ ID NO: 20)
VDLRTLDVFSGCGGLSEGFHQAGISDTLWAIEMWDPAAQAFRLNNPGSTV
FTEDCNILLKLVMAGETTNSRGQRLPQKGDVEMLCGGPPCQGFSGMNRFN
SRTYSKFKNSLVVSFLSYCDYYRPRFFLLENVRNFVSFKRSMVLKLTLRC
LVRMGYQCTFGVLQAGQYGVAQTRRRAIILAAAPGEKLPLFPEPLHVFAP
RACQLSVVVDDKKFVSNITRLSSGPFRTITVRDTMSDLPEVRNGASALEI
SYNGEPQSWFQRQLRGAQYQPILRDHICKDMSALVAARMRHIPLAPGSDW
RDLPNIEVRLSDGTMARKLRYTHHDRKNGRSSSGALRGVCSCVEAGKACD
PAARQFNTLIPWCLPHTGNRHNHWAGLYGRLEWDGFFSTTVTNPEPMGKQ
GRVLHPEQHRVVSVRECARSQGFPDTYRLFGNILDKHRQVGNAVPPPLAK
AIGLEIKLCMLAKARESASAKIKEEEAAKD

In some embodiments, DNMT1 is encoded by a nucleotide sequence of SEQ ID NO: 53. In some embodiments, a nucleic acid described herein comprises a sequence of SEQ ID NO: 53 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto

(SEQ ID NO: 53)
GTGGATCTGAGGACACTCGACGTGTTTAGCGGATGCGGCGGACTCTCCGA
AGGCTTCCACCAAGCCGGAATTTCCGACACACTCTGGGCCATTGAGATGT
GGGACCCCGCCGCTCAAGCCTTCAGACTGAATAATCCCGGCTCCACCGTG
TTCACCGAGGACTGCAACATTCTGCTGAAGCTGGTGATGGCTGGCGAAAC
CACCAACTCTAGAGGCCAGAGGCTGCCCCAGAAGGGAGATGTGGAAATGC
TCTGTGGAGGCCCTCCTTGCCAAGGCTTCTCCGGCATGAACAGGTTCAAC
TCTAGAACATACAGCAAGTTCAAGAACTCTCTGGTCGTGAGCTTTCTGAG
CTACTGCGACTACTATAGACCTAGGTTCTTTCTGCTGGAGAACGTGAGAA
ATTTCGTGTCCTTCAAGAGGAGCATGGTGCTGAAGCTGACACTGAGGTGT
CTGGTGAGGATGGGCTACCAGTGCACATTCGGAGTGCTGCAAGCTGGCCA
GTACGGCGTGGCCCAGACCAGAAGGAGGGCCATCATTCTGGCTGCTGCCC
CCGGCGAGAAACTCCCTCTGTTCCCCGAGCCCCTCCACGTGTTCGCCCCT
AGAGCTTGCCAGCTGAGCGTGGTGGTCGACGATAAGAAGTTCGTGAGCAA
CATCACAAGGCTGTCCAGCGGACCCTTCAGAACCATTACCGTGAGGGATA
CCATGTCCGACCTCCCCGAGGTGAGGAATGGCGCCAGCGCTCTGGAGATT
TCCTACAACGGCGAACCTCAGAGCTGGTTCCAAAGGCAGCTGAGAGGCGC
TCAGTATCAGCCCATTCTGAGGGACCACATCTGCAAAGATATGAGCGCTC
TGGTGGCCGCTAGAATGAGACATATTCCTCTGGCCCCCGGCAGCGACTGG
AGAGATCTGCCCAATATTGAGGTGAGACTCAGCGACGGAACAATGGCTAG
AAAACTGAGGTACACCCATCATGATAGAAAGAACGGAAGGAGCAGCAGCG
GCGCTCTGAGAGGAGTGTGTAGCTGCGTGGAAGCTGGCAAGGCTTGCGAT
CCCGCCGCTAGGCAGTTCAATACCCTCATCCCTTGGTGTCTGCCTCACAC
CGGCAACAGACACAATCATTGGGCTGGACTGTATGGAAGGCTCGAATGGG
ACGGCTTTTTCAGCACCACCGTGACCAATCCCGAACCTATGGGCAAGCAA
GGAAGGGTGCTCCACCCCGAGCAGCATAGAGTCGTGTCCGTGAGAGAATG
CGCTAGAAGCCAAGGCTTCCCCGACACCTATAGACTGTTCGGCAACATTC
TGGATAAGCACAGACAAGTGGGAAATGCTGTCCCTCCTCCTCTGGCCAAG
GCTATCGGACTGGAGATCAAGCTGTGTATGCTCGCCAAAGCTAGGGAGAG
CGCTTCCGCCAAGATTAAGGAGGAGGAGGCCGCCAAGGAC

In some embodiments, DNMT1 for use in a polypeptide or an expression repressor described herein is a variant, e.g., comprising one or more mutations, relative to a DNMT sequence of SEQ ID NO: 20. In some embodiments, the effector domain comprises one or more amino acid substitutions, deletions, or insertions relative to wild type DNMT1. In some embodiments, the polypeptide is a fusion protein comprising a repressor domain that is or comprises DNMT1 and a targeting moiety. In some embodiments, the targeting moiety is or comprises a zinc finger domain, TAL domain, or CRISPR/Cas domain, e.g., a dCas9 domain. In some embodiments, an expression repression system comprises two or more (e.g., two, three, or four) expression repressors, wherein the first expression repressor comprises an effector moiety comprising DNMT1, or a functional variant or fragment thereof.

In another aspect, the disclosure is directed to an expression repressor or a polypeptide comprising one or more (e.g., one) targeting moiety and one or more effector moiety, wherein the one or more effector moiety is or comprises DNMT3a/3L complex, or a functional variant or fragment thereof. In some embodiments, the DNMT3a/3L complex fusion construct. In some embodiments the DNMT3a/3L complex comprises DNMT3A (e.g., human DNMT3A) (e.g., as according to NP_072046.2 or the protein encoded by NM_022552.4). In some embodiments the DNMT3a/3L complex comprises DNMT3L (e.g., as according to NP_787063.1 or the protein encoded by NM_175867.3). In some embodiments, DNMT3a/3L comprises an amino acid sequence of SEQ ID NO: 21 or SEQ ID NO: 114. In some embodiments, an effector domain described herein comprises SEQ ID NO: 21 or SEQ ID NO: 114, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 21)
EWGPFDLVIGGSPCNDLSIVNPARKGLYEGTGRLFFEFYRLLHDARPKEG
DDRPFFWLFENVVAMGVSDKRDISRFLESNPVMIDAKEVSAAHRARYFWG
NLPGMNRPLASTVNDKLELQECLEHGRIAKFSKVRTITTRSNSIKQGKDQ
HFPVFMNEKEDILWCTEMERVFGFPVHYTDVSNMSRLARQRLLGRSWSVP
VIRHLFAPLKEYFACVSSGNSNANSRGPSFSSGLVPLSLRGSHMNPLEMF
ETVPVWRRQPVRVLSLFEDIKKELTSLGFLESGSDPGQLKHVVDVTDTVR
KDVEEWGPFDLVYGATPPLGHTCDRPPSWYLFQFHRLLQYARPKPGSPRP
FFWMFVDNLVLNKEDLDVASRFLEMEPVTIPDVHGGSLQNAVRVWSNIPA
IRSRHWALVSEEELSLLAQNKQSSKLAAKWPTKLVKNCFLPLREYFKYFS
TELTSSL
(SEQ ID NO: 114)
NHDQEFDPPKVYPPVPAEKRKPIRVLSLFDGIATGLLVLKDLGIQVDRYI
ASEVCEDSITVGMVRHQGKIMYVGDVRSVTQKHIQEWGPFDLVIGGSPCN
DLSIVNPARKGLYEGTGRLFFEFYRLLHDARPKEGDDRPFFWLFENVVAM
GVSDKRDISRFLESNPVMIDAKEVSAAHRARYFWGNLPGMNRPLASTVND
KLELQECLEHGRIAKFSKVRTITTRSNSIKQGKDQHFPVFMNEKEDILWC
TEMERVFGFPVHYTDVSNMSRLARQRLLGRSWSVPVIRHLFAPLKEYFAC
VSSGNSNANSRGPSFSSGLVPLSLRGSHMNPLEMFETVPVWRRQPVRVLS
LFEDIKKELTSLGFLESGSDPGQLKHVVDVTDTVRKDVEEWGPFDLVYGA
TPPLGHTCDRPPSWYLFQFHRLLQYARPKPGSPRPFFWMFVDNLVLNKED
LDVASRFLEMEPVTIPDVHGGSLQNAVRVWSNIPAIRSRHWALVSEEELS
LLAQNKQSSKLAAKWPTKLVKNCFLPLREYFKYFSTELTSSL

In some embodiments, DNMT3a/3L is encoded by a nucleotide sequence of SEQ ID NO: 54. In some embodiments, a nucleic acid described herein comprises a sequence of SEQ ID NO: 54 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 54)
AACCACGACCAGGAGTTCGACCCCCCCAAGGTGTACCCCCCCGTGCCCGC
CGAGAAGCGGAAGCCCATCCGGGTGCTGAGCCTGTTCGACGGCATCGCCA
CCGGCCTGCTGGTGCTGAAGGACCTGGGCATCCAGGTGGACCGGTACATC
GCCAGCGAGGTGTGCGAGGACAGCATCACCGTGGGCATGGTGCGGCACCA
GGGCAAGATCATGTACGTGGGCGACGTGCGGAGCGTGACCCAGAAGCACA
TCCAGGAGTGGGGCCCCTTCGACCTGGTGATCGGCGGCAGCCCCTGCAAC
GACCTGAGCATCGTGAACCCCGCCCGGAAGGGCCTGTACGAGGGCACCGG
CCGGCTGTTCTTCGAGTTCTACCGGCTGCTGCACGACGCCCGGCCCAAGG
AGGGCGACGACCGGCCCTTCTTCTGGCTGTTCGAGAACGTGGTGGCCATG
GGCGTGAGCGACAAGCGGGACATCAGCCGGTTCCTGGAGAGCAACCCCGT
GATGATCGACGCCAAGGAGGTGAGCGCCGCCCACCGGGCCCGGTACTTCT
GGGGCAACCTGCCCGGCATGAACCGGCCCCTGGCCAGCACCGTGAACGAC
AAGCTGGAGCTGCAGGAGTGCCTGGAGCACGGCCGGATCGCCAAGTTCAG
CAAGGTGCGGACCATCACCACCCGGAGCAACAGCATCAAGCAGGGCAAGG
ACCAGCACTTCCCCGTGTTCATGAACGAGAAGGAGGACATCCTGTGGTGC
ACCGAGATGGAGCGGGTGTTCGGCTTCCCCGTGCACTACACCGACGTGAG
CAACATGAGCCGGCTGGCCCGGCAGCGGCTGCTGGGCCGGAGCTGGAGCG
TGCCCGTGATCCGGCACCTGTTCGCCCCCCTGAAGGAGTACTTCGCCTGC
GTGAGCAGCGGCAACAGCAACGCCAACAGCCGGGGCCCCAGCTTCAGCAG
CGGCCTGGTGCCCCTGAGCCTGCGGGGCAGCCACATGAATCCTCTGGAGA
TGTTCGAGACAGTGCCCGTGTGGAGAAGGCAACCCGTGAGGGTGCTGAGC
CTCTTCGAGGACATTAAGAAGGAGCTGACCTCTCTGGGCTTTCTGGAATC
CGGCAGCGACCCCGGCCAGCTGAAACACGTGGTGGACGTGACCGACACAG
TGAGGAAGGACGTGGAAGAGTGGGGCCCCTTTGACCTCGTGTATGGAGCC
ACACCTCCTCTCGGCCACACATGCGATAGGCCTCCCAGCTGGTATCTCTT
CCAGTTCCACAGACTGCTCCAGTACGCCAGACCTAAGCCCGGCAGCCCCA
GACCCTTCTTCTGGATGTTCGTGGACAATCTGGTGCTGAACAAGGAGGAT
CTGGATGTGGCCAGCAGATTTCTGGAGATGGAACCCGTGACAATCCCCGA
CGTGCATGGCGGCTCTCTGCAGAACGCCGTGAGAGTGTGGTCCAACATCC
CCGCCATTAGAAGCAGACACTGGGCTCTGGTGAGCGAGGAGGAACTGTCT
CTGCTGGCCCAGAATAAGCAGTCCTCCAAGCTGGCCGCCAAGTGGCCCAC
CAAGCTGGTGAAGAACTGCTTTCTGCCTCTGAGGGAGTATTTCAAGTATT
TCAGCACCGAACTGACCAGCAGCCTG

In some embodiments, DNMT3a/3L for use in a polypeptide or an expression repressor described herein is a variant, e.g., comprising one or more mutations, relative to the DNMT3a/3L of SEQ ID NO: 21 or SEQ ID NO: 114. In some embodiments, an DNMT3a/3L variant comprises one or more amino acid substitutions, deletions, or insertions relative to SEQ ID NO: 21 or SEQ ID NO: 114. In some embodiments, the polypeptide or the expression repressor is a fusion protein comprising an effector moiety that is or comprises DNMT3a/3L and a targeting moiety. In some embodiments, the targeting moiety is or comprises a zinc finger domain, TAL domain, or CRISPR/Cas domain e.g., a dCas9 domain. In some embodiments, an expression repression system comprises two or more (e.g., two, three, or four) expression repressors, wherein the first expression repressor comprises an effector moiety comprising DNMT3a/3L, or a functional variant or fragment thereof.

In some embodiments, an effector moiety is or comprises a polypeptide. In some embodiments, an effector moiety is or comprises a nucleic acid. In some embodiments, an effector moiety is a chemical, e.g., a chemical that modulates a cytosine I or an adenine(A) (e.g., Na bisulfite, ammonium bisulfite). In some embodiments, an effector moiety has enzymatic activity (e.g., methyl transferase, demethylase, nuclease (e.g., Cas9), or deaminase activity). An effector moiety may be or comprise one or more of a small molecule, a peptide, a nucleic acid, a nanoparticle, an aptamer, or a pharmaco-agent with poor PK/PD.

In some embodiments, an effector moiety, may comprise a peptide ligand, a full-length protein, a protein fragment, an antibody, an antibody fragment, and/or a targeting aptamer. In some embodiments, the protein may bind a receptor such as an extracellular receptor, neuropeptide, hormone peptide, peptide drug, toxic peptide, viral or microbial peptide, synthetic peptide, or agonist or antagonist peptide.

In some embodiments, an effector moiety may comprise antigens, antibodies, antibody fragments such as, e.g. single domain antibodies, ligands, or receptors such as, e.g., glucagon-like peptide-1 (GLP-1), GLP-2 receptor 2, cholecystokinin B (CCKB), or somatostatin receptor, peptide therapeutics such as, e.g., those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, or degradation or self-destruction peptides such as an apoptosis-inducing peptide signal or photosensitizer peptide.

Peptide or protein moieties for use in effector moieties as described herein may also include small antigen-binding peptides, e.g., antigen binding antibody or antibody-like fragments, such as, e.g., single chain antibodies, nanobodies (see, e.g., Steeland et al. 2016. Nanobodies as therapeutics: big opportunities for small antibodies. Drug Discov Today: 21(7):1076-113). Such small antigen binding peptides may bind, e.g., a cytosolic antigen, a nuclear antigen, an intra-organellar antigen.

In some embodiments, an effector moiety comprises a dominant negative component (e.g., dominant negative moiety), e.g., a protein that recognizes and binds a sequence (e.g., an anchor sequence, e.g., a CTCF binding motif), but with an inactive (e.g., mutated) dimerization domain, e.g., a dimerization domain that is unable to form a functional anchor sequence-mediated conjunction), or binds to a component of a genomic complex (e.g., a transcription factor subunit, etc.) preventing formation of a functional transcription factor, etc. For example, the Zinc Finger domain of CTCF can be altered so that it binds a specific anchor sequence (by adding zinc fingers that recognize flanking nucleic acids), while the homo-dimerization domain is altered to prevent the interaction between engineered CTCF and endogenous forms of CTCF. In some embodiments, a dominant negative component comprises a synthetic nucleating polypeptide with a selected binding affinity for an anchor sequence within a target anchor sequence-mediated conjunction. In some embodiments, binding affinity may be at least 10%, 15%, 20%, 25%, 30%, 35%, 40%, 45%, 50%, 55%, 60%, 65%, 70%, 75%, 80%, 85%, 90%, 95%, or higher or lower than binding affinity of an endogenous nucleating polypeptide (e.g., CTCF) that associates with a target anchor sequence. A synthetic nucleating polypeptide may have between 30-90%, 30-85%, 30-80%, 30-70%, 50-80%, 50-90% amino acid sequence identity to a corresponding endogenous nucleating polypeptide. A nucleating polypeptide may modulate (e.g., disrupt), such as through competitive binding, e.g., competing with binding of an endogenous nucleating polypeptide to its anchor sequence.

In some embodiments, an effector moiety comprises an antibody or fragment thereof. In some embodiments, target gene (e.g., MYC) expression is altered via use of effector moieties that are or comprise one or more antibodies or fragments thereof. In some embodiments, gene expression is altered via use of effector moieties that are or comprise one or more antibodies (or fragments thereof) and dCas9.

In some embodiments, an antibody or fragment thereof for use in an effector moiety may be monoclonal. An antibody may be a fusion, a chimeric antibody, a non-humanized antibody, a partially or fully humanized antibody, etc. As will be understood by one of skill in the art, format of antibody(ies) used may be the same or different depending on a given target.

In some embodiments, an effector moiety, comprises a conjunction nucleating molecule, a nucleic acid encoding a conjunction nucleating molecule, or a combination thereof. A conjunction nucleating molecule may be, e.g., CTCF, cohesin, USF1, YY1, TATA-box binding protein associated factor 3 (TAF3), ZNF143 binding motif, or another polypeptide that promotes formation of an anchor sequence-mediated conjunction. A conjunction nucleating molecule may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. A conjunction nucleating molecule may modulate DNA interactions within or around the anchor sequence-mediated conjunction (e.g., associated with or comprising the genomic sequence element targeted by the targeting moiety). For example, a conjunction nucleating molecule can recruit other factors to an anchor sequence that alters an anchor sequence-mediated conjunction formation or disruption.

A conjunction nucleating molecule may also have a dimerization domain for homo- or heterodimerization. One or more conjunction nucleating molecules, e.g., endogenous and engineered, may interact to form an anchor sequence-mediated conjunction. In some embodiments, a conjunction nucleating molecule is engineered to further include a stabilization domain, e.g., cohesion interaction domain, to stabilize an anchor sequence-mediated conjunction. In some embodiments, a conjunction nucleating molecule is engineered to bind a target sequence, e.g., target sequence binding affinity is modulated. In some embodiments, a conjunction nucleating molecule is selected or engineered with a selected binding affinity for an anchor sequence within an anchor sequence-mediated conjunction.

Conjunction nucleating molecules and their corresponding anchor sequences may be identified through use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include ChIA-PET analysis using a bait, such as Cohesin, YY1 or USF1, ZNF143 binding motif, and MS to identify complexes that are associated with a bait.

In some embodiments, an effector moiety comprises a DNA-binding domain of a protein. In some embodiments, a DNA binding domain of an effector moiety enhances or alters targeting of a modulating agent but does not alone achieve complete targeting by a modulating agent (e.g., the targeting moiety is still needed to achieve targeting of the modulating agent). In some embodiments, a DNA binding domain enhances targeting of a modulating agent. In some embodiments, a DNA binding domain enhances efficacy of a modulating agent. DNA-binding proteins have distinct structural motifs, e.g., that play a key role in binding DNA, known to those of skill in the art. In some embodiments, a DNA-binding domain comprises a helix-turn-helix (HTH) motif, a common DNA recognition motif in repressor proteins. Such a motif comprises two helices, one of which recognizes DNA (aka recognition helix) with side chains providing binding specificity. Such motifs are commonly used to regulate proteins that are involved in developmental processes. Sometimes more than one protein competes for the same sequence or recognizes the same DNA fragment. Different proteins may differ in their affinity for the same sequence, or DNA conformation, respectively through H-bonds, salt bridges and Van der Waals interactions.

In some embodiments, a DNA-binding domain comprises a helix-hairpin-helix (HhH) motif. DNA-binding proteins with a HhH structural motif may be involved in non-sequence-specific DNA binding that occurs via the formation of hydrogen bonds between protein backbone nitrogen and DNA phosphate groups.

In some embodiments, a DNA-binding domain comprises a helix-loop-helix (HLH) motif. DNA-binding proteins with an HLH structural motif are transcriptional regulatory proteins and are principally related to a wide array of developmental processes. An HLH structural motif is longer, in terms of residues, than HTH or HhH motifs. Many of these proteins interact to form homo- and hetero-dimers. A structural motif is composed of two long helix regions, with an N-terminal helix binding to DNA, while a complex region allows the protein to dimerize.

In some embodiments, a DNA-binding domain comprises a leucine zipper motif. In some transcription factors, a dimer binding site with DNA forms a leucine zipper. This motif includes two amphipathic helices, one from each subunit, interacting with each other resulting in a left-handed coiled-coil super secondary structure. A leucine zipper is an interdigitation of regularly spaced leucine residues in one helix with leucines from an adjacent helix. Mostly, helices involved in leucine zippers exhibit a heptad sequence (abcdefg) with residues a and d being hydrophobic and other residues being hydrophilic. Leucine zipper motifs can mediate either homo- or heterodimer formation.

In some embodiments, a DNA-binding domain comprises a Zn finger domain, where a Ze⁺⁺ ion is coordinated by 2 Cys and 2 His residues. Such a transcription factor includes a trimer with the stoichiometry ββ′α. An apparent effect of Zn⁺⁺ coordination is stabilization of a small complex structure instead of hydrophobic core residues. Each Zn-finger interacts in a conformationally identical manner with successive triple base pair segments in the major groove of the double helix. Protein-DNA interaction is determined by two factors: (i) H-bonding interaction between α-helix and DNA segment, mostly between Arg residues and Guanine bases. (ii) H-bonding interaction with DNA phosphate backbone, mostly with Arg and His. An alternative Zn-finger motif chelates Zn⁺⁺ with 6 Cys.

In some embodiments, a DNA-binding domain comprises a TATA box binding protein (TBP). TBP was first identified as a component of the class II initiation factor TFIID. These binding proteins participate in transcription by all three nuclear RNA polymerases acting as subunit in each of them. Structure of TBP shows two α/β structural domains of 89-90 amino acids. The C-terminal or core region of TBP binds with high affinity to a TATA consensus sequence (TATAa/tAa/t, SEQ ID NO: 210) recognizing minor groove determinants and promoting DNA bending. TBP resemble a molecular saddle. The binding side is lined with central 8 strands of a 10-stranded anti-parallel β-sheet. The upper surface contains four α-helices and binds to various components of transcription machinery.

In some embodiments, a DNA-binding domain is or comprises a transcription factor. Transcription factors (TFs) may be modular proteins containing a DNA-binding domain that is responsible for specific recognition of base sequences and one or more effector domains that can activate or repress transcription. TFs interact with chromatin and recruit protein complexes that serve as coactivators or corepressors.

In some embodiments, an effector moiety comprises one or more RNAs (e.g., gRNA) and dCas9. In some embodiments, one or more RNAs is/are targeted to a genomic sequence element via dCas9 and target-specific guide RNA. As will be understood by one of skill in the art, RNAs used for targeting may be the same or different depending on a given target. An effector moiety may comprise an aptamer, such as an oligonucleotide aptamer or a peptide aptamer. Aptamer moieties are oligonucleotide or peptide aptamers.

An effector moiety may comprise an oligonucleotide aptamer. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition and can be produced by chemical synthesis. In addition, aptamers possess desirable storage properties, and elicit little or no immunogenicity in therapeutic applications.

Both DNA and RNA aptamers show robust binding affinities for various targets. For example, DNA and RNA aptamers have been selected for t lysozyme, thrombin, human immunodeficiency virus trans-acting responsive element (HIV TAR), hemin, interferon γ, vascular endothelial growth factor (VEGF), prostate specific antigen (PSA), dopamine, and the non-classical oncogene, heat shock factor 1 (HSF1).

Diagnostic techniques for aptamer-based plasma protein profiling includes aptamer plasma proteomics. This technology will enable future multi-biomarker protein measurements that can aid diagnostic distinction of disease versus healthy states.

An effector moiety may comprise a peptide aptamer moiety. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide complexes of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer complex attached to a transcription factor binding domain is screened against a target protein attached to a transcription factor activating domain. In vivo binding of a peptide aptamer to its target via this selection strategy is detected as expression of a downstream yeast marker gene. Such experiments identify particular proteins bound by aptamers, and protein interactions that aptamers disrupt, to cause a given phenotype. In addition, peptide aptamers derivatized with appropriate functional moieties can cause specific post-translational modification of their target proteins or change subcellular localization of the targets. Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently a yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. Peptides panned from combinatorial peptide libraries have been stored in a special database with named MimoDB.

An exemplary effector moiety may include, but is not limited to: ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3A, DNMT3B, DNMT3a/3L, MQ1), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdbl), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), helicases such as DHX9, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl β-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), specific domains from proteins, such as a KRAB domain, and fusions of one or more thereof (e.g., dCas9-DNMT, dCas9-MQ1, dCas9-KRAB).

In some embodiments, a candidate domain may be determined to be suitable for use as an effector moiety by methods known to those of skill in the art. For example, a candidate effector moiety may be tested by assaying whether, when the candidate effector moiety is present in the nucleus of a cell and appropriately localized (e.g., to a target gene or transcription control element operably linked to said target gene, e.g., via a targeting moiety), the candidate effector moiety decreases expression of the target gene in the cell, e.g., decreases the level of RNA transcript encoded by the target gene (e.g., as measured by RNASeq or Northern blot) or decreases the level of protein encoded by the target gene (e.g., as measured by ELISA).

In some embodiments, an expression repressor comprises a plurality of effector moiety, wherein each effector moiety does not detectably bind, e.g., does not bind, to another effector moiety. In some embodiments, an expression repression system comprises a first expression repressor comprising a first effector moiety and a second expression repressor comprising a second effector moiety, wherein the first effector moiety does not detectably bind, e.g., does not bind, to the second effector moiety.

In some embodiments, an expression repression system comprises a plurality of expression repressors, wherein each member of the plurality of expression repressors comprises an effector moiety, wherein each effector moiety does not detectably bind, e.g., does not bind, to another effector moiety. In some embodiments, an expression repression system comprises a first expression repressor comprising a first effector moiety and a second expression repressor comprising a second effector moiety, wherein the first effector moiety does not detectably bind, e.g., does not bind, to the second effector moiety. In some embodiments, an expression repression system comprises a first expression repressor comprising a first effector moiety and a second expression repressor comprising a second effector moiety, wherein the first effector moiety does not detectably bind, e.g., does not bind, to another first effector moiety, and the second effector moiety does not detectably bind, e.g., does not bind, to another second effector moiety. In some embodiments, an effector moiety for use in the compositions and methods described herein is functional in a monomeric, e.g., non-dimeric, state.

In some embodiments, an effector moiety is or comprises an epigenetic modifying moiety, e.g., that modulates the two-dimensional structure of chromatin (i.e., that modulate structure of chromatin in a way that would alter its two-dimensional representation).

Epigenetic modifying moieties useful in methods and compositions of the present disclosure include agents that affect epigenetic markers, e.g., DNA methylation, histone methylation, histone acetylation, histone sumoylation, histone phosphorylation, and RNA-associated silencing. Exemplary epigenetic enzymes that can be targeted to a genomic sequence element as described herein include DNA methylases (e.g., DNMT3a, DNMT3b, DNMT3a/3L, MQ1), DNA demethylation (e.g., the TET family), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, an expression repressor, e.g., comprising an epigenetic modifying moiety, useful herein comprises or is a construct described in Koferle et al. Genome Medicine 7.59 (2015):1-3 incorporated herein by reference. For example, in some embodiments, an expression repressor comprises or is a construct found in Table 1 of Koferle et al., e.g., histone deacetylase, histone methyltransferase, DNA demethylation, or H3K4 and/or H3K9 histone demethylase described in Table 1 (e.g., dCas9-p300, TALE-TET1, ZF-DNMT3A, or TALE-LSD1).

In some embodiments, an effector moiety comprises a component of a gene editing system e.g, a CRISPR/Cas domain, e.g., a Zn Finger domain, e.g., a TAL effector domain. In some embodiments, an epigenetic modifying moiety may comprise a polypeptide (e.g., peptide or protein moiety) linked to a gRNA and a targeted nuclease, e.g., a Cas9, e.g., a wild type Cas9, a nickase Cas9 (e.g., Cas9 D10A), a catalytically inactive Cas9 (dCas9), eSpCas9, Cpf1, C2C1, or C2C3, or a nucleic acid encoding such a nuclease.

As used herein, a “biologically active portion of an effector domain” is a portion that maintains function (e.g., completely, partially, minimally) of an effector domain (e.g., a “minimal” or “core” domain). In some embodiments, fusion of a dCas9 with all or a portion of one or more effector domains of an epigenetic modifying agent (such as a DNA methylase or enzyme with a role in DNA demethylation, e.g., DNMT3a, DNMT3b, DNMT3L, a DNMT inhibitor, combinations thereof, TET family enzymes, protein acetyl transferase or deacetylase, dCas9-DNMT3a/3L, dCas9-DNMT3a/3L/KRAB, dCas9/VP64) creates a chimeric protein that is linked to the polypeptide and useful in the methods described herein. An effector moiety comprising such a chimeric protein is referred to as either a genetic modifying moiety (because of its use of a gene editing system component, Cas9) or an epigenetic modifying moiety (because of its use of an effector domain of an epigenetic modifying agent).

In some embodiments, provided technologies are described as comprising a gRNA that specifically targets a target gene. In some embodiments, the target gene is an oncogene, a tumor suppressor, or a MYC mis-regulation disorder related gene. In some embodiments, the target gene is MYC.

In some embodiments, technologies provided herein include methods of delivering one or more genetic modifying moieties (e.g., CRISPR system components) described herein to a subject, e.g., to a nucleus of a cell or tissue of a subject, by linking such a moiety to a targeting moiety as part of a fusion molecule. In some embodiments, technologies provided herein include methods of delivering one or more genetic modifying moieties (e.g., CRISPR system components) described herein to a subject, e.g., to a nucleus of a cell or tissue of a subject, by encapsulating the one or more genetic modifying moieties (e.g., CRISPR system components) in a lipid nanoparticle.

Additional Moieties

An expression repressor may further comprise one or more additional moieties (e.g., in addition to one or more targeting moieties and one or more effector moieties). In some embodiments, an additional moiety is selected from a tagging or monitoring moiety, a cleavable moiety (e.g., a cleavable moiety positioned between a DNA-targeting moiety and an effector moiety or at the N- or C-terminal end of a polypeptide), a small molecule, a membrane translocating polypeptide, or a pharmaco-agent moiety.

Exemplary Expression Repressors

The following exemplary expression repressors are presented for illustration purposes only and are not intended to be limiting.

In some embodiments, an expression repressor comprises a targeting moiety comprising dCas9, e.g., an S. aureus dCas9, and an effector moiety comprising MQ1, e.g., bacterial MQ1. In some embodiments, the expression repressor is encoded by the nucleic acid sequence of SEQ ID NOs: 68 (e.g., a nucleic acid (e.g., cDNA) encoding the expression repressor). In some embodiments, the expression repressor is encoded by the nucleic acid sequence of SEQ ID NOs: 119. In some embodiments, a nucleic acid described herein comprises a nucleic acid sequence of SEQ ID NO: 68, 119 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-MQ1 nucleotide sequence:
(SEQ ID NO: 68)
GAGTTCGTGTACGGCGACTACAAGGTGTACGACGTGCGGAAGATGATCGCCAAGAGCGAGC
AGGAGATCGGCAAGGCCACCGCCAAGTACTTCTTCTACAGCAACATCATGAACTTCTTCAAG
ACCGAGATCACCCTGGCCAACGGCGAGATCCGGAAGCGGCCCCTGATCGAGACCAACGGCG
AGACCGGCGAGATCGTGTGGGACAAGGGCCGGGACTTCGCCACCGTGCGGAAGGTGCTGAG
CATGCCCCAGGTGAACATCGTGAAGAAAACCGAGGTGCAGACCGGCGGCTTCAGCAAGGAG
AGCATCCTGCCCAAGCGGAACAGCGACAAGCTGATCGCCCGGAAGAAGGACTGGGACCCCA
AGAAGTACGGCGGCTTCGACAGCCCCACCGTGGCCTACAGCGTGCTGGTGGTGGCCAAGGT
GGAGAAGGGCAAGAGCAAGAAGCTGAAATCCGTGAAGGAGCTGCTGGGCATCACCATCATG
GAGCGGAGCAGCTTCGAGAAGAACCCCATCGACTTCCTGGAGGCCAAGGGCTACAAGGAGG
TGAAGAAGGACCTGATCATCAAGCTGCCCAAGTACAGCCTGTTCGAGCTGGAGAACGGCCG
GAAGCGGATGCTGGCCAGCGCCGGCGAGCTGCAGAAGGGCAACGAGCTGGCCCTGCCCAGC
AAGTACGTGAACTTCCTGTACCTGGCCAGCCACTACGAGAAGCTGAAGGGCAGCCCCGAGG
ACAACGAGCAGAAGCAGCTGTTCGTGGAGCAGCACAAGCACTACCTGGACGAGATCATCGA
GCAGATCAGCGAGTTCAGCAAGCGGGTGATCCTGGCCGACGCCAACCTGGACAAGGTGCTG
AGCGCCTACAACAAGCACCGGGACAAGCCCATCCGGGAGCAGGCCGAGAACATCATCCACC
TGTTCACCCTGACCAACCTGGGCGCCCCCGCCGCCTTCAAGTACTTCGACACCACCATCGAC
CGGAAGCGGTACACCAGCACCAAGGAGGTGCTGGACGCCACCCTGATCCACCAGAGCATCA
CCGGCCTGTACGAGACCCGGATCGACCTGAGCCAGCTGGGCGGCGACAAGCGGCCCGCCGC
CACCAAGAAGGCCGGCCAGGCCAAGAAGAAGAAGGCCCGGGACAGCAAGGTGGAGAACAA
GACCAAGAAGCTGCGGGTGTTCGAGGCCTTCGCCGGCATCGGCGCCCAGCGGAAGGCCCTG
GAGAAGGTGCGGAAGGACGAGTACGAGATCGTGGGCCTGGCCGAGTGGTACGTGCCCGCCA
TCGTGATGTACCAGGCCATCCACAACAACTTCCACACCAAGCTGGAGTACAAGAGCGTGAG
CCGGGAGGAGATGATCGACTACCTGGAGAACAAGACCCTGAGCTGGAACAGCAAGAACCCC
GTGAGCAACGGCTACTGGAAGCGGAAGAAGGACGACGAGCTGAAGATCATCTACAACGCCA
TCAAGCTGAGCGAGAAGGAGGGCAACATCTTCGACATCCGGGACCTGTACAAGCGGACCCT
GAAGAACATCGACCTGCTGACCTACAGCTTCCCCTGCCAGGACCTGAGCCAGCAGGGCATCC
AGAAGGGCATGAAGCGGGGCAGCGGCACCCGGAGCGGCCTGCTGTGGGAGATCGAGCGGG
CCCTGGACAGCACCGAGAAGAACGACCTGCCCAAGTACCTGCTGATGGAGAACGTGGGCGC
CCTGCTGCACAAGAAGAACGAGGAGGAGCTGAACCAGTGGAAGCAGAAGCTGGAGAGCCT
GGGCTACCAGAACAGCATCGAGGTGCTGAACGCCGCCGACTTCGGCAGCAGCCAGGCCCGG
CGGCGGGTGTTCATGATCAGCACCCTGAACGAGTTCGTGGAGCTGCCCAAGGGCGACAAGA
AGCCCAAGAGCATCAAGAAGGTGCTGAACAAGATCGTGAGCGAGAAGGACATCCTGAACA
ACCTGCTGAAGTACAACCTGACCGAGTTCAAGAAAACCAAGAGCAACATCAACAAGGCCAG
CCTGATCGGCTACAGCAAGTTCAACAGCGAGGGCTACGTGTACGACCCCGAGTTCACCGGCC
CCACCCTGACCGCCAGCGGCGCCAACAGCCGGATCAAGATCAAGGACGGCAGCAACATCCG
GAAGATGAACAGCGACGAGACCTTCCTGTACATCGGCTTCGACAGCCAGGACGGCAAGCGG
GTGAACGAGATCGAGTTCCTGACCGAGAACCAGAAGATCTTCGTGTGCGGCAACAGCATCA
GCGTGGAGGTGCTGGAGGCCATCATCGACAAGATCGGCGGCCCCAGCAGCGGCGGCAAGCG
GCCCGCCGCCACCAAGAAGGCCGGCCAGGCCAAGAAGAAGAAGGGCAGCTACCCCTACGA
CGTGCCCGACTACGCCTGAGCGGCCGCTTAATTAAGCTGCCTTCTGCGGGGCTTGCCTTCTG
GCCATGCCCTTCTTCTCTCCCTTGCACCTGTACCTCTTGGTCTTTGAATAAAGCCTGAGTAGG
AAGTCTAGAAAAAAAAAAAAAAAAAAAAAAAAAAAAAAAAAAAAAAAAAAAAAAAAAAA
AAAAAAAAAAAAAAAAAAAAAAAAAAAAA
(SEQ ID NO: 119)
AAGGAAATAAGAGAGAAAAGAAGAGTAAGAAGAAATATAAGAGCCACCATGGCCCCCAAG
AAGAAGCGGAAGGTGGGCATCCACGGCGTGCCCGCCGCCGACAAGAAGTACAGCATCGGCC
TGGCCATCGGCACCAACAGCGTGGGCTGGGCCGTGATCACCGACGAGTACAAGGTGCCCAG
CAAGAAGTTCAAGGTGCTGGGCAACACCGACCGGCACAGCATCAAGAAGAACCTGATCGGC
GCCCTGCTGTTCGACAGCGGCGAGACCGCCGAGGCCACCCGGCTGAAGCGGACCGCCCGGC
GGCGGTACACCCGGCGGAAGAACCGGATCTGCTACCTGCAGGAGATCTTCAGCAACGAGAT
GGCCAAGGTGGACGACAGCTTCTTCCACCGGCTGGAGGAGAGCTTCCTGGTGGAGGAGGAC
AAGAAGCACGAGCGGCACCCCATCTTCGGCAACATCGTGGACGAGGTGGCCTACCACGAGA
AGTACCCCACCATCTACCACCTGCGGAAGAAGCTGGTGGACAGCACCGACAAGGCCGACCT
GCGGCTGATCTACCTGGCCCTGGCCCACATGATCAAGTTCCGGGGCCACTTCCTGATCGAGG
GCGACCTGAACCCCGACAACAGCGACGTGGACAAGCTGTTCATCCAGCTGGTGCAGACCTA
CAACCAGCTGTTCGAGGAGAACCCCATCAACGCCAGCGGCGTGGACGCCAAGGCCATCCTG
AGCGCCCGGCTGAGCAAGAGCCGGCGGCTGGAGAACCTGATCGCCCAGCTGCCCGGCGAGA
AGAAGAACGGCCTGTTCGGCAACCTGATCGCCCTGAGCCTGGGCCTGACCCCCAACTTCAAG
AGCAACTTCGACCTGGCCGAGGACGCCAAGCTGCAGCTGAGCAAGGACACCTACGACGACG
ACCTGGACAACCTGCTGGCCCAGATCGGCGACCAGTACGCCGACCTGTTCCTGGCCGCCAAG
AACCTGAGCGACGCCATCCTGCTGAGCGACATCCTGCGGGTGAACACCGAGATCACCAAGG
CCCCCCTGAGCGCCAGCATGATCAAGCGGTACGACGAGCACCACCAGGACCTGACCCTGCT
GAAGGCCCTGGTGCGGCAGCAGCTGCCCGAGAAGTACAAGGAGATCTTCTTCGACCAGAGC
AAGAACGGCTACGCCGGCTACATCGACGGCGGCGCCAGCCAGGAGGAGTTCTACAAGTTCA
TCAAGCCCATCCTGGAGAAGATGGACGGCACCGAGGAGCTGCTGGTGAAGCTGAACCGGGA
GGACCTGCTGCGGAAGCAGCGGACCTTCGACAACGGCAGCATCCCCCACCAGATCCACCTG
GGCGAGCTGCACGCCATCCTGCGGCGGCAGGAGGACTTCTACCCCTTCCTGAAGGACAACC
GGGAGAAGATCGAGAAGATCCTGACCTTCCGGATCCCCTACTACGTGGGCCCCCTGGCCCGG
GGCAACAGCCGGTTCGCCTGGATGACCCGGAAATCCGAGGAGACCATCACCCCCTGGAACT
TCGAGGAGGTGGTGGACAAGGGCGCCAGCGCCCAGAGCTTCATCGAGCGGATGACCAACTT
CGACAAGAACCTGCCCAACGAGAAGGTGCTGCCCAAGCACAGCCTGCTGTACGAGTACTTC
ACCGTGTACAACGAGCTGACCAAGGTGAAGTACGTGACCGAGGGCATGCGGAAGCCCGCCT
TCCTGAGCGGCGAGCAGAAGAAGGCCATCGTGGACCTGCTGTTCAAGACCAACCGGAAGGT
GACCGTGAAGCAGCTGAAGGAGGACTACTTCAAGAAGATCGAGTGCTTCGACAGCGTGGAG
ATCAGCGGCGTGGAGGACCGGTTCAACGCCAGCCTGGGCACCTACCACGACCTGCTGAAGA
TCATCAAGGACAAGGACTTCCTGGACAACGAGGAGAACGAGGACATCCTGGAGGACATCGT
GCTGACCCTGACCCTGTTCGAGGACCGGGAGATGATCGAGGAGCGGCTGAAAACCTACGCC
CACCTGTTCGACGACAAGGTGATGAAGCAGCTGAAGCGGCGGCGGTACACCGGCTGGGGCC
GGCTGAGCCGGAAGCTGATCAACGGCATCCGGGACAAGCAGAGCGGCAAGACCATCCTGGA
CTTCCTGAAATCCGACGGCTTCGCCAACCGGAACTTCATGCAGCTGATCCACGACGACAGCC
TGACCTTCAAGGAGGACATCCAGAAGGCCCAGGTGAGCGGCCAGGGCGACAGCCTGCACGA
GCACATCGCCAACCTGGCCGGCAGCCCCGCCATCAAGAAGGGCATCCTGCAGACCGTGAAG
GTGGTGGACGAGCTGGTGAAGGTGATGGGCCGGCACAAGCCCGAGAACATCGTGATCGAGA
TGGCCCGGGAGAACCAGACCACCCAGAAGGGCCAGAAGAACAGCCGGGAGCGGATGAAGC
GGATCGAGGAGGGCATCAAGGAGCTGGGCAGCCAGATCCTGAAGGAGCACCCCGTGGAGA
ACACCCAGCTGCAGAACGAGAAGCTGTACCTGTACTACCTGCAGAACGGCCGGGACATGTA
CGTGGACCAGGAGCTGGACATCAACCGGCTGAGCGACTACGACGTGGCCGCCATCGTGCCC
CAGAGCTTCCTGAAGGACGACAGCATCGACAACAAGGTGCTGACCCGGAGCGACAAGGCCC
GGGGCAAGAGCGACAACGTGCCCAGCGAGGAGGTGGTGAAGAAGATGAAGAACTACTGGC
GGCAGCTGCTGAACGCCAAGCTGATCACCCAGCGGAAGTTCGACAACCTGACCAAGGCCGA
GCGGGGCGGCCTGAGCGAGCTGGACAAGGCCGGCTTCATCAAGCGGCAGCTGGTGGAGACC
CGGCAGATCACCAAGCACGTGGCCCAGATCCTGGACAGCCGGATGAACACCAAGTACGACG
AGAACGACAAGCTGATCCGGGAGGTGAAGGTGATCACCCTGAAATCCAAGCTGGTGAGCGA
CTTCCGGAAGGACTTCCAGTTCTACAAGGTGCGGGAGATCAACAACTACCACCACGCCCACG
ACGCCTACCTGAACGCCGTGGTGGGCACCGCCCTGATCAAGAAGTACCCCAAGCTGGAGAG
CGAGTTCGTGTACGGCGACTACAAGGTGTACGACGTGCGGAAGATGATCGCCAAGAGCGAG
CAGGAGATCGGCAAGGCCACCGCCAAGTACTTCTTCTACAGCAACATCATGAACTTCTTCAA
GACCGAGATCACCCTGGCCAACGGCGAGATCCGGAAGCGGCCCCTGATCGAGACCAACGGC
GAGACCGGCGAGATCGTGTGGGACAAGGGCCGGGACTTCGCCACCGTGCGGAAGGTGCTGA
GCATGCCCCAGGTGAACATCGTGAAGAAAACCGAGGTGCAGACCGGCGGCTTCAGCAAGGA
GAGCATCCTGCCCAAGCGGAACAGCGACAAGCTGATCGCCCGGAAGAAGGACTGGGACCCC
AAGAAGTACGGCGGCTTCGACAGCCCCACCGTGGCCTACAGCGTGCTGGTGGTGGCCAAGG
TGGAGAAGGGCAAGAGCAAGAAGCTGAAATCCGTGAAGGAGCTGCTGGGCATCACCATCAT
GGAGCGGAGCAGCTTCGAGAAGAACCCCATCGACTTCCTGGAGGCCAAGGGCTACAAGGAG
GTGAAGAAGGACCTGATCATCAAGCTGCCCAAGTACAGCCTGTTCGAGCTGGAGAACGGCC
GGAAGCGGATGCTGGCCAGCGCCGGCGAGCTGCAGAAGGGCAACGAGCTGGCCCTGCCCAG
CAAGTACGTGAACTTCCTGTACCTGGCCAGCCACTACGAGAAGCTGAAGGGCAGCCCCGAG
GACAACGAGCAGAAGCAGCTGTTCGTGGAGCAGCACAAGCACTACCTGGACGAGATCATCG
AGCAGATCAGCGAGTTCAGCAAGCGGGTGATCCTGGCCGACGCCAACCTGGACAAGGTGCT
GAGCGCCTACAACAAGCACCGGGACAAGCCCATCCGGGAGCAGGCCGAGAACATCATCCAC
CTGTTCACCCTGACCAACCTGGGCGCCCCCGCCGCCTTCAAGTACTTCGACACCACCATCGA
CCGGAAGCGGTACACCAGCACCAAGGAGGTGCTGGACGCCACCCTGATCCACCAGAGCATC
ACCGGCCTGTACGAGACCCGGATCGACCTGAGCCAGCTGGGCGGCGACAAGCGGCCCGCCG
CCACCAAGAAGGCCGGCCAGGCCAAGAAGAAGAAGGCCCGGGACAGCAAGGTGGAGAACA
AGACCAAGAAGCTGCGGGTGTTCGAGGCCTTCGCCGGCATCGGCGCCCAGCGGAAGGCCCT
GGAGAAGGTGCGGAAGGACGAGTACGAGATCGTGGGCCTGGCCGAGTGGTACGTGCCCGCC
ATCGTGATGTACCAGGCCATCCACAACAACTTCCACACCAAGCTGGAGTACAAGAGCGTGA
GCCGGGAGGAGATGATCGACTACCTGGAGAACAAGACCCTGAGCTGGAACAGCAAGAACCC
CGTGAGCAACGGCTACTGGAAGCGGAAGAAGGACGACGAGCTGAAGATCATCTACAACGCC
ATCAAGCTGAGCGAGAAGGAGGGCAACATCTTCGACATCCGGGACCTGTACAAGCGGACCC
TGAAGAACATCGACCTGCTGACCTACAGCTTCCCCTGCCAGGACCTGAGCCAGCAGGGCATC
CAGAAGGGCATGAAGCGGGGCAGCGGCACCCGGAGCGGCCTGCTGTGGGAGATCGAGCGG
GCCCTGGACAGCACCGAGAAGAACGACCTGCCCAAGTACCTGCTGATGGAGAACGTGGGCG
CCCTGCTGCACAAGAAGAACGAGGAGGAGCTGAACCAGTGGAAGCAGAAGCTGGAGAGCC
TGGGCTACCAGAACAGCATCGAGGTGCTGAACGCCGCCGACTTCGGCAGCAGCCAGGCCCG
GCGGCGGGTGTTCATGATCAGCACCCTGAACGAGTTCGTGGAGCTGCCCAAGGGCGACAAG
AAGCCCAAGAGCATCAAGAAGGTGCTGAACAAGATCGTGAGCGAGAAGGACATCCTGAAC
AACCTGCTGAAGTACAACCTGACCGAGTTCAAGAAAACCAAGAGCAACATCAACAAGGCCA
GCCTGATCGGCTACAGCAAGTTCAACAGCGAGGGCTACGTGTACGACCCCGAGTTCACCGG
CCCCACCCTGACCGCCAGCGGCGCCAACAGCCGGATCAAGATCAAGGACGGCAGCAACATC
CGGAAGATGAACAGCGACGAGACCTTCCTGTACATCGGCTTCGACAGCCAGGACGGCAAGC
GGGTGAACGAGATCGAGTTCCTGACCGAGAACCAGAAGATCTTCGTGTGCGGCAACAGCAT
CAGCGTGGAGGTGCTGGAGGCCATCATCGACAAGATCGGCGGCCCCAGCAGCGGCGGCAAG
CGGCCCGCCGCCACCAAGAAGGCCGGCCAGGCCAAGAAGAAGAAGGGCAGCTACCCCTAC
GACGTGCCCGACTACGCCTGAGCGGCCGCTTAATTAAGCTGCCTTCTGCGGGGCTTGCCTTC
TGGCCATGCCCTTCTTCTCTCCCTTGCACCTGTACCTCTTGGTCTTTGAATAAAGCCTGAGTA
GGAAGTCTAGAAAAAAAAAAAAAAAAAAAAAAAAAAAAAAAAAAAAAAAAAAAAAAAAA
AAAAAAAAAAAAAAAAAAAAAAAAAAAAAAA

In some embodiments, an expression repressor comprises the amino acid sequence of SEQ ID NOs: 35 or 151. In some embodiments, an expression repressor described herein comprises an amino acid sequence of SEQ ID NO: 35, 151, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-MQ1 Protein sequence:
(SEQ ID NO: 35)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDKRPAATKKAGQAKKKKARDSKVENKTKKLRVFEAFAGIGAQRKALE
KVRKDEYEIVGLAEWYVPAIVMYQAIHNNFHTKLEYKSVSREEMIDYLENKTLSWNSKNPVSN
GYWKRKKDDELKIIYNAIKLSEKEGNIFDIRDLYKRTLKNIDLLTYSFPCQDLSQQGIQKGMKRG
SGTRSGLLWEIERALDSTEKNDLPKYLLMENVGALLHKKNEEELNQWKQKLESLGYQNSIEVLN
AADFGSSQARRRVFMISTLNEFVELPKGDKKPKSIKKVLNKIVSEKDILNNLLKYNLTEFKKTKS
NINKASLIGYSKFNSEGYVYDPEFTGPTLTASGANSRIKIKDGSNIRKMNSDETFLYIGFDSQDGK
RVNEIEFLTENQKIFVCGNSISVEVLEAIIDKIGGPSSGGKRPAATKKAGQAKKKKGSYPYDVPDY
A
(SEQ ID NO: 151)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDKRPAATKKAGQAKKKKARDSKVENKTKKLRVFEAFAGIGAQRKALE
KVRKDEYEIVGLAEWYVPAIVMYQAIHNNFHTKLEYKSVSREEMIDYLENKTLSWNSKNPVSN
GYWKRKKDDELKIIYNAIKLSEKEGNIFDIRDLYKRTLKNIDLLTYSFPCQDLSQQGIQKGMKRG
SGTRSGLLWEIERALDSTEKNDLPKYLLMENVGALLHKKNEEELNQWKQKLESLGYQNSIEVLN
AADFGSSQARRRVFMISTLNEFVELPKGDKKPKSIKKVLNKIVSEKDILNNLLKYNLTEFKKTKS
NINKASLIGYSKFNSEGYVYDPEFTGPTLTASGANSRIKIKDGSNIRKMNSDETFLYIGFDSQDGK
RVNEIEFLTENQKIFVCGNSISVEVLEAIIDKIGGPSSGGKRPAATKKAGQAKKKKGS

In some embodiments, an expression repressor comprises a targeting moiety comprising dCas9, e.g., an S. pyogenes dCas9, and an effector moiety comprising KRAB, e.g., a KRAB domain. In some embodiments, the expression repressor is encoded by the nucleic acid sequence of SEQ ID NOs: 67 (e.g., a nucleic acid (e.g., cDNA) encoding the expression repressor). In some embodiments, a nucleic acid described herein comprises a nucleic acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-KRAB nucleotide sequence:
(SEQ ID NO: 67)
AAGGAAATAAGAGAGAAAAGAAGAGTAAGAAGAAATATAAGAGCCACCATGGCCCCCAAG
AAGAAGCGGAAGGTGGGCATCCACGGCGTGCCCGCCGCCGACAAGAAGTACAGCATCGGCC
TGGCCATCGGCACCAACAGCGTGGGCTGGGCCGTGATCACCGACGAGTACAAGGTGCCCAG
CAAGAAGTTCAAGGTGCTGGGCAACACCGACCGGCACAGCATCAAGAAGAACCTGATCGGC
GCCCTGCTGTTCGACAGCGGCGAGACCGCCGAGGCCACCCGGCTGAAGCGGACCGCCCGGC
GGCGGTACACCCGGCGGAAGAACCGGATCTGCTACCTGCAGGAGATCTTCAGCAACGAGAT
GGCCAAGGTGGACGACAGCTTCTTCCACCGGCTGGAGGAGAGCTTCCTGGTGGAGGAGGAC
AAGAAGCACGAGCGGCACCCCATCTTCGGCAACATCGTGGACGAGGTGGCCTACCACGAGA
AGTACCCCACCATCTACCACCTGCGGAAGAAGCTGGTGGACAGCACCGACAAGGCCGACCT
GCGGCTGATCTACCTGGCCCTGGCCCACATGATCAAGTTCCGGGGCCACTTCCTGATCGAGG
GCGACCTGAACCCCGACAACAGCGACGTGGACAAGCTGTTCATCCAGCTGGTGCAGACCTA
CAACCAGCTGTTCGAGGAGAACCCCATCAACGCCAGCGGCGTGGACGCCAAGGCCATCCTG
AGCGCCCGGCTGAGCAAGAGCCGGCGGCTGGAGAACCTGATCGCCCAGCTGCCCGGCGAGA
AGAAGAACGGCCTGTTCGGCAACCTGATCGCCCTGAGCCTGGGCCTGACCCCCAACTTCAAG
AGCAACTTCGACCTGGCCGAGGACGCCAAGCTGCAGCTGAGCAAGGACACCTACGACGACG
ACCTGGACAACCTGCTGGCCCAGATCGGCGACCAGTACGCCGACCTGTTCCTGGCCGCCAAG
AACCTGAGCGACGCCATCCTGCTGAGCGACATCCTGCGGGTGAACACCGAGATCACCAAGG
CCCCCCTGAGCGCCAGCATGATCAAGCGGTACGACGAGCACCACCAGGACCTGACCCTGCT
GAAGGCCCTGGTGCGGCAGCAGCTGCCCGAGAAGTACAAGGAGATCTTCTTCGACCAGAGC
AAGAACGGCTACGCCGGCTACATCGACGGCGGCGCCAGCCAGGAGGAGTTCTACAAGTTCA
TCAAGCCCATCCTGGAGAAGATGGACGGCACCGAGGAGCTGCTGGTGAAGCTGAACCGGGA
GGACCTGCTGCGGAAGCAGCGGACCTTCGACAACGGCAGCATCCCCCACCAGATCCACCTG
GGCGAGCTGCACGCCATCCTGCGGCGGCAGGAGGACTTCTACCCCTTCCTGAAGGACAACC
GGGAGAAGATCGAGAAGATCCTGACCTTCCGGATCCCCTACTACGTGGGCCCCCTGGCCCGG
GGCAACAGCCGGTTCGCCTGGATGACCCGGAAATCCGAGGAGACCATCACCCCCTGGAACT
TCGAGGAGGTGGTGGACAAGGGCGCCAGCGCCCAGAGCTTCATCGAGCGGATGACCAACTT
CGACAAGAACCTGCCCAACGAGAAGGTGCTGCCCAAGCACAGCCTGCTGTACGAGTACTTC
ACCGTGTACAACGAGCTGACCAAGGTGAAGTACGTGACCGAGGGCATGCGGAAGCCCGCCT
TCCTGAGCGGCGAGCAGAAGAAGGCCATCGTGGACCTGCTGTTCAAGACCAACCGGAAGGT
GACCGTGAAGCAGCTGAAGGAGGACTACTTCAAGAAGATCGAGTGCTTCGACAGCGTGGAG
ATCAGCGGCGTGGAGGACCGGTTCAACGCCAGCCTGGGCACCTACCACGACCTGCTGAAGA
TCATCAAGGACAAGGACTTCCTGGACAACGAGGAGAACGAGGACATCCTGGAGGACATCGT
GCTGACCCTGACCCTGTTCGAGGACCGGGAGATGATCGAGGAGCGGCTGAAAACCTACGCC
CACCTGTTCGACGACAAGGTGATGAAGCAGCTGAAGCGGCGGCGGTACACCGGCTGGGGCC
GGCTGAGCCGGAAGCTGATCAACGGCATCCGGGACAAGCAGAGCGGCAAGACCATCCTGGA
CTTCCTGAAATCCGACGGCTTCGCCAACCGGAACTTCATGCAGCTGATCCACGACGACAGCC
TGACCTTCAAGGAGGACATCCAGAAGGCCCAGGTGAGCGGCCAGGGCGACAGCCTGCACGA
GCACATCGCCAACCTGGCCGGCAGCCCCGCCATCAAGAAGGGCATCCTGCAGACCGTGAAG
GTGGTGGACGAGCTGGTGAAGGTGATGGGCCGGCACAAGCCCGAGAACATCGTGATCGAGA
TGGCCCGGGAGAACCAGACCACCCAGAAGGGCCAGAAGAACAGCCGGGAGCGGATGAAGC
GGATCGAGGAGGGCATCAAGGAGCTGGGCAGCCAGATCCTGAAGGAGCACCCCGTGGAGA
ACACCCAGCTGCAGAACGAGAAGCTGTACCTGTACTACCTGCAGAACGGCCGGGACATGTA
CGTGGACCAGGAGCTGGACATCAACCGGCTGAGCGACTACGACGTGGCCGCCATCGTGCCC
CAGAGCTTCCTGAAGGACGACAGCATCGACAACAAGGTGCTGACCCGGAGCGACAAGGCCC
GGGGCAAGAGCGACAACGTGCCCAGCGAGGAGGTGGTGAAGAAGATGAAGAACTACTGGC
GGCAGCTGCTGAACGCCAAGCTGATCACCCAGCGGAAGTTCGACAACCTGACCAAGGCCGA
GCGGGGCGGCCTGAGCGAGCTGGACAAGGCCGGCTTCATCAAGCGGCAGCTGGTGGAGACC
CGGCAGATCACCAAGCACGTGGCCCAGATCCTGGACAGCCGGATGAACACCAAGTACGACG
AGAACGACAAGCTGATCCGGGAGGTGAAGGTGATCACCCTGAAATCCAAGCTGGTGAGCGA
CTTCCGGAAGGACTTCCAGTTCTACAAGGTGCGGGAGATCAACAACTACCACCACGCCCACG
ACGCCTACCTGAACGCCGTGGTGGGCACCGCCCTGATCAAGAAGTACCCCAAGCTGGAGAG
CGAGTTCGTGTACGGCGACTACAAGGTGTACGACGTGCGGAAGATGATCGCCAAGAGCGAG
CAGGAGATCGGCAAGGCCACCGCCAAGTACTTCTTCTACAGCAACATCATGAACTTCTTCAA
GACCGAGATCACCCTGGCCAACGGCGAGATCCGGAAGCGGCCCCTGATCGAGACCAACGGC
GAGACCGGCGAGATCGTGTGGGACAAGGGCCGGGACTTCGCCACCGTGCGGAAGGTGCTGA
GCATGCCCCAGGTGAACATCGTGAAGAAAACCGAGGTGCAGACCGGCGGCTTCAGCAAGGA
GAGCATCCTGCCCAAGCGGAACAGCGACAAGCTGATCGCCCGGAAGAAGGACTGGGACCCC
AAGAAGTACGGCGGCTTCGACAGCCCCACCGTGGCCTACAGCGTGCTGGTGGTGGCCAAGG
TGGAGAAGGGCAAGAGCAAGAAGCTGAAATCCGTGAAGGAGCTGCTGGGCATCACCATCAT
GGAGCGGAGCAGCTTCGAGAAGAACCCCATCGACTTCCTGGAGGCCAAGGGCTACAAGGAG
GTGAAGAAGGACCTGATCATCAAGCTGCCCAAGTACAGCCTGTTCGAGCTGGAGAACGGCC
GGAAGCGGATGCTGGCCAGCGCCGGCGAGCTGCAGAAGGGCAACGAGCTGGCCCTGCCCAG
CAAGTACGTGAACTTCCTGTACCTGGCCAGCCACTACGAGAAGCTGAAGGGCAGCCCCGAG
GACAACGAGCAGAAGCAGCTGTTCGTGGAGCAGCACAAGCACTACCTGGACGAGATCATCG
AGCAGATCAGCGAGTTCAGCAAGCGGGTGATCCTGGCCGACGCCAACCTGGACAAGGTGCT
GAGCGCCTACAACAAGCACCGGGACAAGCCCATCCGGGAGCAGGCCGAGAACATCATCCAC
CTGTTCACCCTGACCAACCTGGGCGCCCCCGCCGCCTTCAAGTACTTCGACACCACCATCGA
CCGGAAGCGGTACACCAGCACCAAGGAGGTGCTGGACGCCACCCTGATCCACCAGAGCATC
ACCGGCCTGTACGAGACCCGGATCGACCTGAGCCAGCTGGGCGGCGACAAGCGGCCCGCCG
CCACCAAGAAGGCCGGCCAGGCCAAGAAGAAGAAGGCCAGCGACGCCAAGAGCCTGACCG
CCTGGAGCCGGACCCTGGTGACCTTCAAGGACGTGTTCGTGGACTTCACCCGGGAGGAGTGG
AAGCTGCTGGACACCGCCCAGCAGATCCTGTACCGGAACGTGATGCTGGAGAACTACAAGA
ACCTGGTGAGCCTGGGCTACCAGCTGACCAAGCCCGACGTGATCCTGCGGCTGGAGAAGGG
CGAGGAGCCCTGGCTGGTGGAGCGGGAGATCCACCAGGAGACCCACCCCGACAGCGAGACC
GCCTTCGAGATCAAGAGCAGCGTGAGCGGCGGCAAGCGGCCCGCCGCCACCAAGAAGGCCG
GCCAGGCCAAGAAGAAGAAGGGCAGCTACCCCTACGACGTGCCCGACTACGCCTGAGCGGC
CGCTTAATTAAGCTGCCTTCTGCGGGGCTTGCCTTCTGGCCATGCCCTTCTTCTCTCCCTTGCA
CCTGTACCTCTTGGTCTTTGAATAAAGCCTGAGTAGGAAGTCTAGAAAAAAAAAAAAAAAA
AAAAAAAAAAAAAAAAAAAAAAAAAAAAAAAAAAAAAAAAAAAAAAAAAAAAAAAAAA
AAAAAA

In some embodiments, an expression repressor comprises the amino acid sequence of SEQ ID NOs: 34 or 150. In some embodiments, a nucleic acid described herein comprises an amino acid sequence of SEQ ID NO: 34, 150, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-KRAB Protein sequence:
(SEQ ID NO: 34)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDKRPAATKKAGQAKKKKASDAKSLTAWSRTLVTFKDVFVDFTREEWK
LLDTAQQILYRNVMLENYKNLVSLGYQLTKPDVILRLEKGEEPWLVEREIHQETHPDSETAFEIK
SSVSGGKRPAATKKAGQAKKKKGSYPYDVPDYA
(SEQ ID NO: 150)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDKRPAATKKAGQAKKKKASDAKSLTAWSRTLVTFKDVFVDFTREEWK
LLDTAQQILYRNVMLENYKNLVSLGYQLTKPDVILRLEKGEEPWLVEREIHQETHPDSETAFEIK
SSVSGGKRPAATKKAGQAKKKKGS

In some embodiments, an expression repressor comprises a DNA-targeting moiety comprising dCas9, e.g., an S. aureus dCas9, and an effector moiety comprising DNMT1, e.g., human DNMT1. In some embodiments, the expression repressor is encoded by the nucleic acid sequence of SEQ ID NO: 69 (e.g., a nucleic acid (e.g., cDNA) encoding the expression repressor). In some embodiments, a nucleic acid described herein comprises a nucleic acid sequence of SEQ ID NO: 69 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT1 nucleotide sequence
(SEQ ID NO: 69)
AGGAAATAAGAGAGAAAAGAAGAGTAAGAAGAAATATAAGAGCCACCATGGCCCCCAAGA
AGAAGCGGAAGGTGGGCATCCACGGCGTGCCCGCCGCCGACAAGAAGTACAGCATCGGCCT
GGCCATCGGCACCAACAGCGTGGGCTGGGCCGTGATCACCGACGAGTACAAGGTGCCCAGC
AAGAAGTTCAAGGTGCTGGGCAACACCGACCGGCACAGCATCAAGAAGAACCTGATCGGCG
CCCTGCTGTTCGACAGCGGCGAGACCGCCGAGGCCACCCGGCTGAAGCGGACCGCCCGGCG
GCGGTACACCCGGCGGAAGAACCGGATCTGCTACCTGCAGGAGATCTTCAGCAACGAGATG
GCCAAGGTGGACGACAGCTTCTTCCACCGGCTGGAGGAGAGCTTCCTGGTGGAGGAGGACA
AGAAGCACGAGCGGCACCCCATCTTCGGCAACATCGTGGACGAGGTGGCCTACCACGAGAA
GTACCCCACCATCTACCACCTGCGGAAGAAGCTGGTGGACAGCACCGACAAGGCCGACCTG
CGGCTGATCTACCTGGCCCTGGCCCACATGATCAAGTTCCGGGGCCACTTCCTGATCGAGGG
CGACCTGAACCCCGACAACAGCGACGTGGACAAGCTGTTCATCCAGCTGGTGCAGACCTAC
AACCAGCTGTTCGAGGAGAACCCCATCAACGCCAGCGGCGTGGACGCCAAGGCCATCCTGA
GCGCCCGGCTGAGCAAGAGCCGGCGGCTGGAGAACCTGATCGCCCAGCTGCCCGGCGAGAA
GAAGAACGGCCTGTTCGGCAACCTGATCGCCCTGAGCCTGGGCCTGACCCCCAACTTCAAGA
GCAACTTCGACCTGGCCGAGGACGCCAAGCTGCAGCTGAGCAAGGACACCTACGACGACGA
CCTGGACAACCTGCTGGCCCAGATCGGCGACCAGTACGCCGACCTGTTCCTGGCCGCCAAGA
ACCTGAGCGACGCCATCCTGCTGAGCGACATCCTGCGGGTGAACACCGAGATCACCAAGGC
CCCCCTGAGCGCCAGCATGATCAAGCGGTACGACGAGCACCACCAGGACCTGACCCTGCTG
AAGGCCCTGGTGCGGCAGCAGCTGCCCGAGAAGTACAAGGAGATCTTCTTCGACCAGAGCA
AGAACGGCTACGCCGGCTACATCGACGGCGGCGCCAGCCAGGAGGAGTTCTACAAGTTCAT
CAAGCCCATCCTGGAGAAGATGGACGGCACCGAGGAGCTGCTGGTGAAGCTGAACCGGGAG
GACCTGCTGCGGAAGCAGCGGACCTTCGACAACGGCAGCATCCCCCACCAGATCCACCTGG
GCGAGCTGCACGCCATCCTGCGGCGGCAGGAGGACTTCTACCCCTTCCTGAAGGACAACCG
GGAGAAGATCGAGAAGATCCTGACCTTCCGGATCCCCTACTACGTGGGCCCCCTGGCCCGGG
GCAACAGCCGGTTCGCCTGGATGACCCGGAAATCCGAGGAGACCATCACCCCCTGGAACTT
CGAGGAGGTGGTGGACAAGGGCGCCAGCGCCCAGAGCTTCATCGAGCGGATGACCAACTTC
GACAAGAACCTGCCCAACGAGAAGGTGCTGCCCAAGCACAGCCTGCTGTACGAGTACTTCA
CCGTGTACAACGAGCTGACCAAGGTGAAGTACGTGACCGAGGGCATGCGGAAGCCCGCCTT
CCTGAGCGGCGAGCAGAAGAAGGCCATCGTGGACCTGCTGTTCAAGACCAACCGGAAGGTG
ACCGTGAAGCAGCTGAAGGAGGACTACTTCAAGAAGATCGAGTGCTTCGACAGCGTGGAGA
TCAGCGGCGTGGAGGACCGGTTCAACGCCAGCCTGGGCACCTACCACGACCTGCTGAAGAT
CATCAAGGACAAGGACTTCCTGGACAACGAGGAGAACGAGGACATCCTGGAGGACATCGTG
CTGACCCTGACCCTGTTCGAGGACCGGGAGATGATCGAGGAGCGGCTGAAAACCTACGCCC
ACCTGTTCGACGACAAGGTGATGAAGCAGCTGAAGCGGCGGCGGTACACCGGCTGGGGCCG
GCTGAGCCGGAAGCTGATCAACGGCATCCGGGACAAGCAGAGCGGCAAGACCATCCTGGAC
TTCCTGAAATCCGACGGCTTCGCCAACCGGAACTTCATGCAGCTGATCCACGACGACAGCCT
GACCTTCAAGGAGGACATCCAGAAGGCCCAGGTGAGCGGCCAGGGCGACAGCCTGCACGAG
CACATCGCCAACCTGGCCGGCAGCCCCGCCATCAAGAAGGGCATCCTGCAGACCGTGAAGG
TGGTGGACGAGCTGGTGAAGGTGATGGGCCGGCACAAGCCCGAGAACATCGTGATCGAGAT
GGCCCGGGAGAACCAGACCACCCAGAAGGGCCAGAAGAACAGCCGGGAGCGGATGAAGCG
GATCGAGGAGGGCATCAAGGAGCTGGGCAGCCAGATCCTGAAGGAGCACCCCGTGGAGAA
CACCCAGCTGCAGAACGAGAAGCTGTACCTGTACTACCTGCAGAACGGCCGGGACATGTAC
GTGGACCAGGAGCTGGACATCAACCGGCTGAGCGACTACGACGTGGCCGCCATCGTGCCCC
AGAGCTTCCTGAAGGACGACAGCATCGACAACAAGGTGCTGACCCGGAGCGACAAGGCCCG
GGGCAAGAGCGACAACGTGCCCAGCGAGGAGGTGGTGAAGAAGATGAAGAACTACTGGCG
GCAGCTGCTGAACGCCAAGCTGATCACCCAGCGGAAGTTCGACAACCTGACCAAGGCCGAG
CGGGGCGGCCTGAGCGAGCTGGACAAGGCCGGCTTCATCAAGCGGCAGCTGGTGGAGACCC
GGCAGATCACCAAGCACGTGGCCCAGATCCTGGACAGCCGGATGAACACCAAGTACGACGA
GAACGACAAGCTGATCCGGGAGGTGAAGGTGATCACCCTGAAATCCAAGCTGGTGAGCGAC
TTCCGGAAGGACTTCCAGTTCTACAAGGTGCGGGAGATCAACAACTACCACCACGCCCACG
ACGCCTACCTGAACGCCGTGGTGGGCACCGCCCTGATCAAGAAGTACCCCAAGCTGGAGAG
CGAGTTCGTGTACGGCGACTACAAGGTGTACGACGTGCGGAAGATGATCGCCAAGAGCGAG
CAGGAGATCGGCAAGGCCACCGCCAAGTACTTCTTCTACAGCAACATCATGAACTTCTTCAA
GACCGAGATCACCCTGGCCAACGGCGAGATCCGGAAGCGGCCCCTGATCGAGACCAACGGC
GAGACCGGCGAGATCGTGTGGGACAAGGGCCGGGACTTCGCCACCGTGCGGAAGGTGCTGA
GCATGCCCCAGGTGAACATCGTGAAGAAAACCGAGGTGCAGACCGGCGGCTTCAGCAAGGA
GAGCATCCTGCCCAAGCGGAACAGCGACAAGCTGATCGCCCGGAAGAAGGACTGGGACCCC
AAGAAGTACGGCGGCTTCGACAGCCCCACCGTGGCCTACAGCGTGCTGGTGGTGGCCAAGG
TGGAGAAGGGCAAGAGCAAGAAGCTGAAATCCGTGAAGGAGCTGCTGGGCATCACCATCAT
GGAGCGGAGCAGCTTCGAGAAGAACCCCATCGACTTCCTGGAGGCCAAGGGCTACAAGGAG
GTGAAGAAGGACCTGATCATCAAGCTGCCCAAGTACAGCCTGTTCGAGCTGGAGAACGGCC
GGAAGCGGATGCTGGCCAGCGCCGGCGAGCTGCAGAAGGGCAACGAGCTGGCCCTGCCCAG
CAAGTACGTGAACTTCCTGTACCTGGCCAGCCACTACGAGAAGCTGAAGGGCAGCCCCGAG
GACAACGAGCAGAAGCAGCTGTTCGTGGAGCAGCACAAGCACTACCTGGACGAGATCATCG
AGCAGATCAGCGAGTTCAGCAAGCGGGTGATCCTGGCCGACGCCAACCTGGACAAGGTGCT
GAGCGCCTACAACAAGCACCGGGACAAGCCCATCCGGGAGCAGGCCGAGAACATCATCCAC
CTGTTCACCCTGACCAACCTGGGCGCCCCCGCCGCCTTCAAGTACTTCGACACCACCATCGA
CCGGAAGCGGTACACCAGCACCAAGGAGGTGCTGGACGCCACCCTGATCCACCAGAGCATC
ACCGGCCTGTACGAGACCCGGATCGACCTGAGCCAGCTGGGCGGCGACAGCGGCGGCAAGC
GGCCCGCCGCCACCAAGAAGGCCGGCCAGGCCAAGAAGAAGAAGTCGGGCGGGGGTGGCT
CAGTGGATCTGAGGACACTCGACGTGTTTAGCGGATGCGGCGGACTCTCCGAAGGCTTCCAC
CAAGCCGGAATTTCCGACACACTCTGGGCCATTGAGATGTGGGACCCCGCCGCTCAAGCCTT
CAGACTGAATAATCCCGGCTCCACCGTGTTCACCGAGGACTGCAACATTCTGCTGAAGCTGG
TGATGGCTGGCGAAACCACCAACTCTAGAGGCCAGAGGCTGCCCCAGAAGGGAGATGTGGA
AATGCTCTGTGGAGGCCCTCCTTGCCAAGGCTTCTCCGGCATGAACAGGTTCAACTCTAGAA
CATACAGCAAGTTCAAGAACTCTCTGGTCGTGAGCTTTCTGAGCTACTGCGACTACTATAGA
CCTAGGTTCTTTCTGCTGGAGAACGTGAGAAATTTCGTGTCCTTCAAGAGGAGCATGGTGCT
GAAGCTGACACTGAGGTGTCTGGTGAGGATGGGCTACCAGTGCACATTCGGAGTGCTGCAA
GCTGGCCAGTACGGCGTGGCCCAGACCAGAAGGAGGGCCATCATTCTGGCTGCTGCCCCCG
GCGAGAAACTCCCTCTGTTCCCCGAGCCCCTCCACGTGTTCGCCCCTAGAGCTTGCCAGCTG
AGCGTGGTGGTCGACGATAAGAAGTTCGTGAGCAACATCACAAGGCTGTCCAGCGGACCCT
TCAGAACCATTACCGTGAGGGATACCATGTCCGACCTCCCCGAGGTGAGGAATGGCGCCAG
CGCTCTGGAGATTTCCTACAACGGCGAACCTCAGAGCTGGTTCCAAAGGCAGCTGAGAGGC
GCTCAGTATCAGCCCATTCTGAGGGACCACATCTGCAAAGATATGAGCGCTCTGGTGGCCGC
TAGAATGAGACATATTCCTCTGGCCCCCGGCAGCGACTGGAGAGATCTGCCCAATATTGAGG
TGAGACTCAGCGACGGAACAATGGCTAGAAAACTGAGGTACACCCATCATGATAGAAAGAA
CGGAAGGAGCAGCAGCGGCGCTCTGAGAGGAGTGTGTAGCTGCGTGGAAGCTGGCAAGGCT
TGCGATCCCGCCGCTAGGCAGTTCAATACCCTCATCCCTTGGTGTCTGCCTCACACCGGCAA
CAGACACAATCATTGGGCTGGACTGTATGGAAGGCTCGAATGGGACGGCTTTTTCAGCACCA
CCGTGACCAATCCCGAACCTATGGGCAAGCAAGGAAGGGTGCTCCACCCCGAGCAGCATAG
AGTCGTGTCCGTGAGAGAATGCGCTAGAAGCCAAGGCTTCCCCGACACCTATAGACTGTTCG
GCAACATTCTGGATAAGCACAGACAAGTGGGAAATGCTGTCCCTCCTCCTCTGGCCAAGGCT
ATCGGACTGGAGATCAAGCTGTGTATGCTCGCCAAAGCTAGGGAGAGCGCTTCCGCCAAGA
TTAAGGAGGAGGAGGCCGCCAAGGACGGAGGTGGCGGATCGGGAAAGCGGCCCGCCGCCA
CCAAGAAGGCCGGTCAGGCCAAGAAGAAGAAGGGCAGCTACCCCTACGACGTGCCCGACTA
CGCCTGAGCGGCCGCTTAATTAAGCTGCCTTCTGCGGGGCTTGCCTTCTGGCCATGCCCTTCT
TCTCTCCCTTGCACCTGTACCTCTTGGTCTTTGAATAAAGCCTGAGTAGGAAGTCTAGAAAA
AAAAAAAAAAAAAAAAAAAAAAAAAAAAAAAAAAAAAAAAAAAAAAAAAAAAAAAAAA
AAAAAAAAAAAAAAAAAA

In some embodiments, an expression repressor comprises the amino acid sequence of SEQ ID NOs: 36, or 152. In some embodiments, an expression repressor described herein comprises an amino acid sequence of SEQ ID NO: 36, 152, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT1 Protein sequence:
(SEQ ID NO: 36)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDSGGKRPAATKKAGQAKKKKSGGGGSVDLRTLDVFSGCGGLSEGFHQ
AGISDTLWAIEMWDPAAQAFRLNNPGSTVFTEDCNILLKLVMAGETTNSRGQRLPQKGDVEML
CGGPPCQGFSGMNRFNSRTYSKFKNSLVVSFLSYCDYYRPRFFLLENVRNFVSFKRSMVLKLTLR
CLVRMGYQCTFGVLQAGQYGVAQTRRRAIILAAAPGEKLPLFPEPLHVFAPRACQLSVVVDDKK
FVSNITRLSSGPFRTITVRDTMSDLPEVRNGASALEISYNGEPQSWFQRQLRGAQYQPILRDHICK
DMSALVAARMRHIPLAPGSDWRDLPNIEVRLSDGTMARKLRYTHHDRKNGRSSSGALRGVCSC
VEAGKACDPAARQFNTLIPWCLPHTGNRHNHWAGLYGRLEWDGFFSTTVTNPEPMGKQGRVL
HPEQHRVVSVRECARSQGFPDTYRLFGNILDKHRQVGNAVPPPLAKAIGLEIKLCMLAKARESAS
AKIKEEEAAKDGGGGSGKRPAATKKAGQAKKKKGSYPYDVPDYA
(SEQ ID NO: 152)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDSGGKRPAATKKAGQAKKKKSGGGGSVDLRTLDVFSGCGGLSEGFHQ
AGISDTLWAIEMWDPAAQAFRLNNPGSTVFTEDCNILLKLVMAGETTNSRGQRLPQKGDVEML
CGGPPCQGFSGMNRFNSRTYSKFKNSLVVSFLSYCDYYRPRFFLLENVRNFVSFKRSMVLKLTLR
CLVRMGYQCTFGVLQAGQYGVAQTRRRAIILAAAPGEKLPLFPEPLHVFAPRACQLSVVVDDKK
FVSNITRLSSGPFRTITVRDTMSDLPEVRNGASALEISYNGEPQSWFQRQLRGAQYQPILRDHICK
DMSALVAARMRHIPLAPGSDWRDLPNIEVRLSDGTMARKLRYTHHDRKNGRSSSGALRGVCSC
VEAGKACDPAARQFNTLIPWCLPHTGNRHNHWAGLYGRLEWDGFFSTTVTNPEPMGKQGRVL
HPEQHRVVSVRECARSQGFPDTYRLFGNILDKHRQVGNAVPPPLAKAIGLEIKLCMLAKARESAS
AKIKEEEAAKDGGGGSGKRPAATKKAGQAKKKKGS

In some embodiments, an expression repressor comprises a DNA-targeting moiety comprising dCas9, e.g., an S. aureus dCas9, and an effector moiety comprising DNMT13a/3L. In some embodiments, the expression repressor is encoded by the nucleic acid sequence of SEQ ID NO: 70 (e.g., a nucleic acid (e.g., cDNA) encoding the expression repressor). In some embodiments, a nucleic acid described herein comprises a nucleic acid sequence of SEQ ID NO: 70 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT3a/3Lnucleotide sequence
(SEQ ID NO: 70)
AGGAAATAAGAGAGAAAAGAAGAGTAAGAAGAAATATAAGAGCCACCATGGCCCCCAAGA
AGAAGCGGAAGGTGGGCATCCACGGCGTGCCCGCCGCCGACAAGAAGTACAGCATCGGCCT
GGCCATCGGCACCAACAGCGTGGGCTGGGCCGTGATCACCGACGAGTACAAGGTGCCCAGC
AAGAAGTTCAAGGTGCTGGGCAACACCGACCGGCACAGCATCAAGAAGAACCTGATCGGCG
CCCTGCTGTTCGACAGCGGCGAGACCGCCGAGGCCACCCGGCTGAAGCGGACCGCCCGGCG
GCGGTACACCCGGCGGAAGAACCGGATCTGCTACCTGCAGGAGATCTTCAGCAACGAGATG
GCCAAGGTGGACGACAGCTTCTTCCACCGGCTGGAGGAGAGCTTCCTGGTGGAGGAGGACA
AGAAGCACGAGCGGCACCCCATCTTCGGCAACATCGTGGACGAGGTGGCCTACCACGAGAA
GTACCCCACCATCTACCACCTGCGGAAGAAGCTGGTGGACAGCACCGACAAGGCCGACCTG
CGGCTGATCTACCTGGCCCTGGCCCACATGATCAAGTTCCGGGGCCACTTCCTGATCGAGGG
CGACCTGAACCCCGACAACAGCGACGTGGACAAGCTGTTCATCCAGCTGGTGCAGACCTAC
AACCAGCTGTTCGAGGAGAACCCCATCAACGCCAGCGGCGTGGACGCCAAGGCCATCCTGA
GCGCCCGGCTGAGCAAGAGCCGGCGGCTGGAGAACCTGATCGCCCAGCTGCCCGGCGAGAA
GAAGAACGGCCTGTTCGGCAACCTGATCGCCCTGAGCCTGGGCCTGACCCCCAACTTCAAGA
GCAACTTCGACCTGGCCGAGGACGCCAAGCTGCAGCTGAGCAAGGACACCTACGACGACGA
CCTGGACAACCTGCTGGCCCAGATCGGCGACCAGTACGCCGACCTGTTCCTGGCCGCCAAGA
ACCTGAGCGACGCCATCCTGCTGAGCGACATCCTGCGGGTGAACACCGAGATCACCAAGGC
CCCCCTGAGCGCCAGCATGATCAAGCGGTACGACGAGCACCACCAGGACCTGACCCTGCTG
AAGGCCCTGGTGCGGCAGCAGCTGCCCGAGAAGTACAAGGAGATCTTCTTCGACCAGAGCA
AGAACGGCTACGCCGGCTACATCGACGGCGGCGCCAGCCAGGAGGAGTTCTACAAGTTCAT
CAAGCCCATCCTGGAGAAGATGGACGGCACCGAGGAGCTGCTGGTGAAGCTGAACCGGGAG
GACCTGCTGCGGAAGCAGCGGACCTTCGACAACGGCAGCATCCCCCACCAGATCCACCTGG
GCGAGCTGCACGCCATCCTGCGGCGGCAGGAGGACTTCTACCCCTTCCTGAAGGACAACCG
GGAGAAGATCGAGAAGATCCTGACCTTCCGGATCCCCTACTACGTGGGCCCCCTGGCCCGGG
GCAACAGCCGGTTCGCCTGGATGACCCGGAAATCCGAGGAGACCATCACCCCCTGGAACTT
CGAGGAGGTGGTGGACAAGGGCGCCAGCGCCCAGAGCTTCATCGAGCGGATGACCAACTTC
GACAAGAACCTGCCCAACGAGAAGGTGCTGCCCAAGCACAGCCTGCTGTACGAGTACTTCA
CCGTGTACAACGAGCTGACCAAGGTGAAGTACGTGACCGAGGGCATGCGGAAGCCCGCCTT
CCTGAGCGGCGAGCAGAAGAAGGCCATCGTGGACCTGCTGTTCAAGACCAACCGGAAGGTG
ACCGTGAAGCAGCTGAAGGAGGACTACTTCAAGAAGATCGAGTGCTTCGACAGCGTGGAGA
TCAGCGGCGTGGAGGACCGGTTCAACGCCAGCCTGGGCACCTACCACGACCTGCTGAAGAT
CATCAAGGACAAGGACTTCCTGGACAACGAGGAGAACGAGGACATCCTGGAGGACATCGTG
CTGACCCTGACCCTGTTCGAGGACCGGGAGATGATCGAGGAGCGGCTGAAAACCTACGCCC
ACCTGTTCGACGACAAGGTGATGAAGCAGCTGAAGCGGCGGCGGTACACCGGCTGGGGCCG
GCTGAGCCGGAAGCTGATCAACGGCATCCGGGACAAGCAGAGCGGCAAGACCATCCTGGAC
TTCCTGAAATCCGACGGCTTCGCCAACCGGAACTTCATGCAGCTGATCCACGACGACAGCCT
GACCTTCAAGGAGGACATCCAGAAGGCCCAGGTGAGCGGCCAGGGCGACAGCCTGCACGAG
CACATCGCCAACCTGGCCGGCAGCCCCGCCATCAAGAAGGGCATCCTGCAGACCGTGAAGG
TGGTGGACGAGCTGGTGAAGGTGATGGGCCGGCACAAGCCCGAGAACATCGTGATCGAGAT
GGCCCGGGAGAACCAGACCACCCAGAAGGGCCAGAAGAACAGCCGGGAGCGGATGAAGCG
GATCGAGGAGGGCATCAAGGAGCTGGGCAGCCAGATCCTGAAGGAGCACCCCGTGGAGAA
CACCCAGCTGCAGAACGAGAAGCTGTACCTGTACTACCTGCAGAACGGCCGGGACATGTAC
GTGGACCAGGAGCTGGACATCAACCGGCTGAGCGACTACGACGTGGCCGCCATCGTGCCCC
AGAGCTTCCTGAAGGACGACAGCATCGACAACAAGGTGCTGACCCGGAGCGACAAGGCCCG
GGGCAAGAGCGACAACGTGCCCAGCGAGGAGGTGGTGAAGAAGATGAAGAACTACTGGCG
GCAGCTGCTGAACGCCAAGCTGATCACCCAGCGGAAGTTCGACAACCTGACCAAGGCCGAG
CGGGGCGGCCTGAGCGAGCTGGACAAGGCCGGCTTCATCAAGCGGCAGCTGGTGGAGACCC
GGCAGATCACCAAGCACGTGGCCCAGATCCTGGACAGCCGGATGAACACCAAGTACGACGA
GAACGACAAGCTGATCCGGGAGGTGAAGGTGATCACCCTGAAATCCAAGCTGGTGAGCGAC
TTCCGGAAGGACTTCCAGTTCTACAAGGTGCGGGAGATCAACAACTACCACCACGCCCACG
ACGCCTACCTGAACGCCGTGGTGGGCACCGCCCTGATCAAGAAGTACCCCAAGCTGGAGAG
CGAGTTCGTGTACGGCGACTACAAGGTGTACGACGTGCGGAAGATGATCGCCAAGAGCGAG
CAGGAGATCGGCAAGGCCACCGCCAAGTACTTCTTCTACAGCAACATCATGAACTTCTTCAA
GACCGAGATCACCCTGGCCAACGGCGAGATCCGGAAGCGGCCCCTGATCGAGACCAACGGC
GAGACCGGCGAGATCGTGTGGGACAAGGGCCGGGACTTCGCCACCGTGCGGAAGGTGCTGA
GCATGCCCCAGGTGAACATCGTGAAGAAAACCGAGGTGCAGACCGGCGGCTTCAGCAAGGA
GAGCATCCTGCCCAAGCGGAACAGCGACAAGCTGATCGCCCGGAAGAAGGACTGGGACCCC
AAGAAGTACGGCGGCTTCGACAGCCCCACCGTGGCCTACAGCGTGCTGGTGGTGGCCAAGG
TGGAGAAGGGCAAGAGCAAGAAGCTGAAATCCGTGAAGGAGCTGCTGGGCATCACCATCAT
GGAGCGGAGCAGCTTCGAGAAGAACCCCATCGACTTCCTGGAGGCCAAGGGCTACAAGGAG
GTGAAGAAGGACCTGATCATCAAGCTGCCCAAGTACAGCCTGTTCGAGCTGGAGAACGGCC
GGAAGCGGATGCTGGCCAGCGCCGGCGAGCTGCAGAAGGGCAACGAGCTGGCCCTGCCCAG
CAAGTACGTGAACTTCCTGTACCTGGCCAGCCACTACGAGAAGCTGAAGGGCAGCCCCGAG
GACAACGAGCAGAAGCAGCTGTTCGTGGAGCAGCACAAGCACTACCTGGACGAGATCATCG
AGCAGATCAGCGAGTTCAGCAAGCGGGTGATCCTGGCCGACGCCAACCTGGACAAGGTGCT
GAGCGCCTACAACAAGCACCGGGACAAGCCCATCCGGGAGCAGGCCGAGAACATCATCCAC
CTGTTCACCCTGACCAACCTGGGCGCCCCCGCCGCCTTCAAGTACTTCGACACCACCATCGA
CCGGAAGCGGTACACCAGCACCAAGGAGGTGCTGGACGCCACCCTGATCCACCAGAGCATC
ACCGGCCTGTACGAGACCCGGATCGACCTGAGCCAGCTGGGCGGCGACAGCGCCGGCGGCG
GCGGCAGCGGCGGCGGCGGCAGCGGCGGCGGCGGCAGCGGCCCCAAGAAGAAGCGGAAGG
TGGCCGCCGCCGGCAGCAACCACGACCAGGAGTTCGACCCCCCCAAGGTGTACCCCCCCGT
GCCCGCCGAGAAGCGGAAGCCCATCCGGGTGCTGAGCCTGTTCGACGGCATCGCCACCGGC
CTGCTGGTGCTGAAGGACCTGGGCATCCAGGTGGACCGGTACATCGCCAGCGAGGTGTGCG
AGGACAGCATCACCGTGGGCATGGTGCGGCACCAGGGCAAGATCATGTACGTGGGCGACGT
GCGGAGCGTGACCCAGAAGCACATCCAGGAGTGGGGCCCCTTCGACCTGGTGATCGGCGGC
AGCCCCTGCAACGACCTGAGCATCGTGAACCCCGCCCGGAAGGGCCTGTACGAGGGCACCG
GCCGGCTGTTCTTCGAGTTCTACCGGCTGCTGCACGACGCCCGGCCCAAGGAGGGCGACGAC
CGGCCCTTCTTCTGGCTGTTCGAGAACGTGGTGGCCATGGGCGTGAGCGACAAGCGGGACAT
CAGCCGGTTCCTGGAGAGCAACCCCGTGATGATCGACGCCAAGGAGGTGAGCGCCGCCCAC
CGGGCCCGGTACTTCTGGGGCAACCTGCCCGGCATGAACCGGCCCCTGGCCAGCACCGTGA
ACGACAAGCTGGAGCTGCAGGAGTGCCTGGAGCACGGCCGGATCGCCAAGTTCAGCAAGGT
GCGGACCATCACCACCCGGAGCAACAGCATCAAGCAGGGCAAGGACCAGCACTTCCCCGTG
TTCATGAACGAGAAGGAGGACATCCTGTGGTGCACCGAGATGGAGCGGGTGTTCGGCTTCC
CCGTGCACTACACCGACGTGAGCAACATGAGCCGGCTGGCCCGGCAGCGGCTGCTGGGCCG
GAGCTGGAGCGTGCCCGTGATCCGGCACCTGTTCGCCCCCCTGAAGGAGTACTTCGCCTGCG
TGAGCAGCGGCAACAGCAACGCCAACAGCCGGGGCCCCAGCTTCAGCAGCGGCCTGGTGCC
CCTGAGCCTGCGGGGCAGCCACATGAATCCTCTGGAGATGTTCGAGACAGTGCCCGTGTGGA
GAAGGCAACCCGTGAGGGTGCTGAGCCTCTTCGAGGACATTAAGAAGGAGCTGACCTCTCT
GGGCTTTCTGGAATCCGGCAGCGACCCCGGCCAGCTGAAACACGTGGTGGACGTGACCGAC
ACAGTGAGGAAGGACGTGGAAGAGTGGGGCCCCTTTGACCTCGTGTATGGAGCCACACCTC
CTCTCGGCCACACATGCGATAGGCCTCCCAGCTGGTATCTCTTCCAGTTCCACAGACTGCTCC
AGTACGCCAGACCTAAGCCCGGCAGCCCCAGACCCTTCTTCTGGATGTTCGTGGACAATCTG
GTGCTGAACAAGGAGGATCTGGATGTGGCCAGCAGATTTCTGGAGATGGAACCCGTGACAA
TCCCCGACGTGCATGGCGGCTCTCTGCAGAACGCCGTGAGAGTGTGGTCCAACATCCCCGCC
ATTAGAAGCAGACACTGGGCTCTGGTGAGCGAGGAGGAACTGTCTCTGCTGGCCCAGAATA
AGCAGTCCTCCAAGCTGGCCGCCAAGTGGCCCACCAAGCTGGTGAAGAACTGCTTTCTGCCT
CTGAGGGAGTATTTCAAGTATTTCAGCACCGAACTGACCAGCAGCCTGAGCGGCGGCAAGC
GGCCCGCCGCCACCAAGAAGGCCGGCCAGGCCAAGAAGAAGAAGGGCAGCTACCCCTACG
ACGTGCCCGACTACGCCTGAGCGGCCGCTTAATTAAGCTGCCTTCTGCGGGGCTTGCCTTCT
GGCCATGCCCTTCTTCTCTCCCTTGCACCTGTACCTCTTGGTCTTTGAATAAAGCCTGAGTAG
GAAGTCTAGAAAAAAAAAAAAAAAAAAAAAAAAAAAAAAAAAAAAAAAAAAAAAAAAAA
AAAAAAAAAAAAAAAAAAAAAAAAAAAAAA

In some embodiments, an expression repressor comprises the amino acid sequence of SEQ ID NO: 37 or 153. In some embodiments, an expression repressor described herein comprises an amino acid sequence of SEQ ID NO: 37 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT3a/3Lprotein sequence
(SEQ ID NO: 37)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDSAGGGGSGGGGSGGGGSGPKKKRKVAAAGSNHDQEFDPPKVYPPVP
AEKRKPIRVLSLFDGIATGLLVLKDLGIQVDRYIASEVCEDSITVGMVRHQGKIMYVGDVRSVTQ
KHIQEWGPFDLVIGGSPCNDLSIVNPARKGLYEGTGRLFFEFYRLLHDARPKEGDDRPFFWLFEN
VVAMGVSDKRDISRFLESNPVMIDAKEVSAAHRARYFWGNLPGMNRPLASTVNDKLELQECLE
HGRIAKFSKVRTITTRSNSIKQGKDQHFPVFMNEKEDILWCTEMERVFGFPVHYTDVSNMSRLAR
QRLLGRSWSVPVIRHLFAPLKEYFACVSSGNSNANSRGPSFSSGLVPLSLRGSHMNPLEMFETVP
VWRRQPVRVLSLFEDIKKELTSLGFLESGSDPGQLKHVVDVTDTVRKDVEEWGPFDLVYGATPP
LGHTCDRPPSWYLFQFHRLLQYARPKPGSPRPFFWMFVDNLVLNKEDLDVASRFLEMEPVTIPD
VHGGSLQNAVRVWSNIPAIRSRHWALVSEEELSLLAQNKQSSKLAAKWPTKLVKNCFLPLREYF
KYFSTELTSSLSGGKRPAATKKAGQAKKKKGSYPYDVPDYA
(SEQ ID NO: 153)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDSAGGGGSGGGGSGGGGSGPKKKRKVAAAGSNHDQEFDPPKVYPPVP
AEKRKPIRVLSLFDGIATGLLVLKDLGIQVDRYIASEVCEDSITVGMVRHQGKIMYVGDVRSVTQ
KHIQEWGPFDLVIGGSPCNDLSIVNPARKGLYEGTGRLFFEFYRLLHDARPKEGDDRPFFWLFEN
VVAMGVSDKRDISRFLESNPVMIDAKEVSAAHRARYFWGNLPGMNRPLASTVNDKLELQECLE
HGRIAKFSKVRTITTRSNSIKQGKDQHFPVFMNEKEDILWCTEMERVFGFPVHYTDVSNMSRLAR
QRLLGRSWSVPVIRHLFAPLKEYFACVSSGNSNANSRGPSFSSGLVPLSLRGSHMNPLEMFETVP
VWRRQPVRVLSLFEDIKKELTSLGFLESGSDPGQLKHVVDVTDTVRKDVEEWGPFDLVYGATPP
LGHTCDRPPSWYLFQFHRLLQYARPKPGSPRPFFWMFVDNLVLNKEDLDVASRFLEMEPVTIPD
VHGGSLQNAVRVWSNIPAIRSRHWALVSEEELSLLAQNKQSSKLAAKWPTKLVKNCFLPLREYF
KYFSTELTSSLSGGKRPAATKKAGQAKKKKGS

In some embodiments, an expression repressor comprises a targeting moiety comprising a Zn Finger domain, and an effector moiety comprising KRAB, e.g., a KRAB domain. In some embodiments, the expression repressors are encoded by a nucleic acid sequence of any of SEQ ID NOs: 55, 56, 57, 58, 59, 60, 189, 194, 195, and 196 (e.g., a nucleic acid (e.g., cDNA) encoding the expression repressor). The nucleic acid sequences of these exemplary expression repressors are disclosed in Table 6. In some embodiments, a nucleic acid described herein comprises a nucleic acid sequence of any of SEQ ID NOs: 55-60, 189, 194-196, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the nucleic acid sequence comprises a poly-A sequence, and in other embodiments, the nucleic acid lacks the poly-A sequence.

TABLE 6
Nucleotide sequences of exemplary ZF-KRAB effectors

| | SEQ | |
|---|---|---|
| | ID | |
| NAME | NO. | SEQUENCE |
| ZF1- | 55 | AGGAAATAAGAGAGAAAAGAAGAGTAAGAAGAAATATAAGAGCCACCAT |
| KRAB | | GGCCCCCAAGAAGAAGCGGAAGGTGGGCATCCACGGCGTGCCCGCCGCC |
| (nt) | | GGCAGCAGCGGATCCCTGGAGCCCGGCGAGAAACCCTACAAGTGCCCCG |
| | | AGTGCGGCAAATCCTTCTCTAGAAGCGACAAACTGACCGAACATCAGAGG |
| | | ACCCACACCGGCGAGAAGCCTTATAAGTGTCCCGAATGCGGCAAATCCTT |
| | | CAGCACCAAGAACTCTCTGACAGAACACCAGAGAACACATACCGGAGAG |
| | | AAACCTTATAAATGCCCCGAGTGCGGCAAGTCCTTCTCCCAGTCCGGCGAT |
| | | CTGAGGAGACACCAAAGAACACATACCGGCGAAAAGCCTTACAAGTGCC |
| | | CCGAGTGTGGAAAGAGCTTCTCCACCACCGGCGCTCTGACCGAGCACCAG |
| | | AGAACACACACCGGCGAGAAACCCTATAAATGTCCCGAGTGTGGCAAATC |
| | | CTTCAGCGACAGCGGCAATCTGAGAGTGCACCAAAGAACCCATACCGGCG |
| | | AAAAACCCTACAAATGCCCCGAGTGCGGCAAATCCTTCAGCCAGAGGGCC |
| | | CATCTGGAGAGGCACCAAAGGACACACACCGGAGAAAAGCCCTACAAGT |
| | | GTCCCGAGTGTGGAAAAAGCTTTAGCACAAGCGGCGAGCTGGTGAGGCAT |
| | | CAAAGGACCCACACCGGCGAAAAGCCCACCGGCAAAAAGACCAGCGCTA |
| | | GCGGCAGCGGCGGCGGCAGCGGCGGCGACGCCAAGAGCCTGACCGCCTG |
| | | GAGCCGGACCCTGGTGACCTTCAAGGACGTGTTCGTGGACTTCACCCGGG |
| | | AGGAGTGGAAGCTGCTGGACACCGCCCAGCAGATCCTGTACCGGAACGTG |
| | | ATGCTGGAGAACTACAAGAACCTGGTGAGCCTGGGCTACCAGCTGACCAA |
| | | GCCCGACGTGATCCTGCGGCTGGAGAAGGGCGAGGAGCCCTGGCTGGTGG |
| | | AGCGGGAGATCCACCAGGAGACCCACCCCGACAGCGAGACCGCCTTCGA |
| | | GATCAAGAGCAGCGTGAGCGGCGGCAAGCGGCCCGCCGCCACCAAGAAG |
| | | GCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTT |
| | | CTGGCCATGCCCTTCTTCTCTCCCTTGCACCTGTACCTCTTGGTCTTTGAAT |
| | | AAAGCCTGAGTAGGAAGTCTAGAAAAAAAAAAAAAAAAAAAAAAAAAA |
| | | AAAAAAAAAAAAAAAAAAAAAAAAAAAAAAAAAAAAAAAAAAAAAAAA |
| | | AAAAAA |
| ZF2- | 56 | AGGAAATAAGAGAGAAAAGAAGAGTAAGAAGAAATATAAGAGCCACCAT |
| KRAB | | GGCCCCCAAGAAGAAGCGGAAGGTGGGCATCCACGGCGTGCCCGCCGCC |
| (nt) | | GGCAGCAGCGGATCCCTGGAGCCCGGCGAGAAGCCCTACAAGTGCCCCG |
| | | AGTGCGGAAAGTCCTTCAGCTCCCCCGCCGATCTGACAAGACATCAGAGA |
| | | ACCCATACCGGCGAGAAACCTTACAAATGCCCCGAATGTGGCAAGTCCTT |
| | | TAGCGATCCCGGACATCTGGTGAGGCACCAGAGGACACACACCGGCGAA |
| | | AAGCCCTATAAATGTCCCGAGTGTGGAAAGAGCTTTTCTAGAAGCGACAA |
| | | TCTCGTGAGACACCAGAGAACCCACACCGGAGAGAAGCCTTACAAGTGCC |
| | | CCGAGTGCGGCAAATCCTTCAGCCAGAGCTCCTCTCTGGTGAGGCACCAA |
| | | AGGACCCACACCGGCGAGAAACCTTATAAGTGTCCCGAGTGTGGCAAAAG |
| | | CTTCAGCACCTCCCACTCTCTGACCGAGCATCAAAGAACCCACACCGGCG |
| | | AAAAACCTTATAAATGCCCCGAGTGTGGCAAATCCTTCAGCAGAAATGAC |
| | | GCTCTGACAGAGCACCAAAGAACACATACCGGAGAAAAGCCCTACAAAT |
| | | GCCCCGAGTGTGGAAAATCCTTTTCTAGAAACGATGCTCTGACCGAACAC |
| | | CAAAGAACACACACCGGCGAAAAGCCTACCGGAAAAAAGACCAGCGCTA |
| | | GCGGCAGCGGCGGCGGCAGCGGCGGCGACGCCAAGAGCCTGACCGCCTG |
| | | GAGCCGGACCCTGGTGACCTTCAAGGACGTGTTCGTGGACTTCACCCGGG |
| | | AGGAGTGGAAGCTGCTGGACACCGCCCAGCAGATCCTGTACCGGAACGTG |
| | | ATGCTGGAGAACTACAAGAACCTGGTGAGCCTGGGCTACCAGCTGACCAA |
| | | GCCCGACGTGATCCTGCGGCTGGAGAAGGGCGAGGAGCCCTGGCTGGTGG |
| | | AGCGGGAGATCCACCAGGAGACCCACCCCGACAGCGAGACCGCCTTCGA |
| | | GATCAAGAGCAGCGTGAGCGGCGGCAAGCGGCCCGCCGCCACCAAGAAG |
| | | GCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTT |
| | | CTGGCCATGCCCTTCTTCTCTCCCTTGCACCTGTACCTCTTGGTCTTTGAAT |
| | | AAAGCCTGAGTAGGAAGTCTAGAAAAAAAAAAAAAAAAAAAAAAAAAA |
| | | AAAAAAAAAAAAAAAAAAAAAAAAAAAAAAAAAAAAAAAAAAAAAAAA |
| | | AAAAAA |
| ZF3- | 57 | AGGAAATAAGAGAGAAAAGAAGAGTAAGAAGAAATATAAGAGCCACCAT |
| KRAB | | GGCCCCCAAGAAGAAGCGGAAGGTGGGCATCCACGGCGTGCCCGCCGCC |
| (nt) | | GGCAGCAGCGGATCCCTGGAGCCCGGCGAAAAACCTTACAAGTGCCCCGA |
| | | GTGCGGAAAGAGCTTCAGCAGAAGCGACAAACTGGTGAGGCATCAAAGG |
| | | ACACATACCGGAGAGAAGCCCTATAAGTGCCCCGAATGTGGCAAATCCTT |
| | | TTCCCAGAGGGCTCATCTGGAAAGACACCAGAGGACCCATACCGGCGAAA |
| | | AACCCTACAAATGTCCCGAGTGTGGAAAGAGCTTTTCCGATCCCGGCCAT |
| | | CTGGTCAGACATCAGAGGACACATACCGGCGAAAAGCCTTACAAGTGTCC |
| | | CGAATGCGGAAAATCCTTCTCCAGAAGCGACAAGCTGGTGAGGCACCAAA |
| | | GAACCCACACCGGCGAAAAACCCTATAAATGCCCCGAGTGCOGCAAGTCC |
| | | TTTAGCCAGCTGGCCCATCTGAGAGCCCACCAGAGAACACACACCGGAGA |
| | | GAAGCCTTATAAGTGTCCCGAGTGCGGAAAGTCCTTCTCTAGAGCCGACA |
| | | ATCTGACCGAACATCAAAGGACACACACCGGCGAGAAACCTTATAAATGC |
| | | CCCGAGTGCGGAAAAAGCTTTTCCGACTGCAGAGATCTGGCTAGACACCA |
| | | GAGAACCCACACCGGCGAGAAACCCACCGGCAAAAAGACCAGCGCTAGC |
| | | GGCAGCGGCGGCGGCAGCGGCGGCGACGCCAAGAGCCTGACCGCCTGGA |
| | | GCCGGACCCTGGTGACCTTCAAGGACGTGTTCGTGGACTTCACCCGGGAG |
| | | GAGTGGAAGCTGCTGGACACCGCCCAGCAGATCCTGTACCGGAACGTGAT |
| | | GCTGGAGAACTACAAGAACCTGGTGAGCCTGGGCTACCAGCTGACCAAGC |
| | | CCGACGTGATCCTGCGGCTGGAGAAGGGCGAGGAGCCCTGGCTGGTGGAG |
| | | CGGGAGATCCACCAGGAGACCCACCCCGACAGCGAGACCGCCTTCGAGAT |
| | | CAAGAGCAGCGTGAGCGGCGGCAAGCGGCCCGCCGCCACCAAGAAGGCC |
| | | GGCCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCTTCTG |
| | | GCCATGCCCTTCTTCTCTCCCTTGCACCTGTACCTCTTGGTCTTTGAATAAA |
| | | GCCTGAGTAGGAAGTCTAGAAAAAAAAAAAAAAAAAAAAAAAAAAAAA |
| | | AAAAAAAAAAAAAAAAAAAAAAAAAAAAAAAAAAAAAAAAAAAAAAAA |
| | | AAA |
| ZF4- | 58 | AGGAAATAAGAGAGAAAAGAAGAGTAAGAAGAAATATAAGAGCCACCATGGCCCCCAAGAAGAAGCGG |
| KRAB | | AAGGTGGGCATCCACGGCGTGCCCGCCGCCGGCAGCAGCGGATCCCTGGAGCCCGGCGAAAAGCCTTAT |
| (nt) | | AAATGTCCCGAATGCGGCAAGAGCTTTAGCCACACCGGCCATCTGCTGGAACACCAAAGGACCCATACC |
| | | GGCGAAAAGCCCTATAAGTGCCCCGAGTGTGGCAAGAGCTTCAGCACCACCGGCAATCTGACAGTCCATC |
| | | AGAGGACCCACACCGGAGAGAAACCCTATAAATGCCCCGAGTGTGGAAAGTCCTTCTCCGACAAGAAGG |
| | | ATCTGACAAGACACCAGAGGACCCATACCGGCGAGAAACCCTACAAATGCCCCGAGTGCGGCAAATCCT |
| | | TCTCCCAGAGCGGCGATCTGAGGAGACATCAAAGAACACATACCGGCGAAAAACCCTATAAGTGCCCCG |
| | | AATGCGGCAAGTCCTTCAGCCAGAGGGCCCATCTGGAAAGGCATCAGAGGACACACACCGGCGAGAAGC |
| | | CTTACAAATGTCCCGAGTGCGGAAAGAGCTTCTCTAGAAGCGACAAGCTGACCGAGCATCAGAGGACCC |
| | | ACACCGGAGAAAAACCTTACAAGTGCCCCGAGTGCGGCAAAAGCTTCAGCAGAACCGACACACTGAGAG |
| | | ATCACCAAAGGACACACACCGGCGAGAAACCCACCGGCAAAAAGACCAGCGCTAGCGGCAGCGGCGGC |
| | | GGCAGCGGCGGCGACGCCAAGAGCCTGACCGCCTGGAGCCGGACCCTGGTGACCTTCAAGGACGTGTTC |
| | | GTGGACTTCACCCGGGAGGAGTGGAAGCTGCTGGACACCGCCCAGCAGATCCTGTACCGGAACGTGATG |
| | | CTGGAGAACTACAAGAACCTGGTGAGCCTGGGCTACCAGCTGACCAAGCCCGACGTGATCCTGCGGCTG |
| | | GAGAAGGGCGAGGAGCCCTGGCTGGTGGAGCGGGAGATCCACCAGGAGACCCACCCCGACAGCGAGAC |
| | | CGCCTTCGAGATCAAGAGCAGCGTGAGCGGCGGCAAGCGGCCCGCCGCCACCAAGAAGGCCGGCCAGGC |
| | | CAAGAAGAAGAAGGGCAGCTACCCCTACGACGTGCCCGACTACGCCTGAGCGGCCGCTTAATTAAGCTG |
| | | CCTTCTGCGGGGCTTGCCTTCTGGCCATGCCCTTCTTCTCTCCCTTGCACCTGTACCTCTTGGTCTTTGAAT |
| | | AAAGCCTGAGTAGGAAGTCTAGAAAAAAAAAAAAAAAAAAAAAAAAAAAAAAAAAAAAAAAAAAAAA |
| | | AAAAAAAAAAAAAAAAAAAAAAAAAAAAAAAAAAA |
| ZF5- | 59 | AGGAAATAAGAGAGAAAAGAAGAGTAAGAAGAAATATAAGAGCCACCAT |
| KRAB | | GGCCCCCAAGAAGAAGCGGAAGGTGGGCATCCACGGCGTGCCCGCCGCC |
| (nt) | | GGCAGCAGCGGATCCCTGGAGCCCGGCGAGAAGCCTTATAAGTGCCCCGA |
| | | GTGTGGCAAGAGCTTTAGCCACACCGGCCATCTGCTGGAGCATCAAAGGA |
| | | CACACACCGGAGAAAAGCCCTATAAGTGCCCCGAGTGTGGCAAATCCTTC |
| | | AGCACCTCCGGCAATCTCACCGAACACCAGAGAACACACACCGGAGAAA |
| | | AACCTTACAAATGTCCCGAGTGTGGAAAGAGCTTTTCCACCAGCGGCAAT |
| | | CTGGTGAGACATCAAAGAACACATACCGGCGAAAAACCCTATAAATGCCC |
| | | CGAGTGTGGAAAATCCTTCTCCCAACTGGCCCATCTGAGGGCCCACCAGA |
| | | GGACACATACCGGAGAAAAACCCTACAAATGCCCCGAATGCGGAAAAAG |
| | | CTTCTCCGAGAGAAGCCATCTGAGAGAGCACCAAAGGACCCATACCGGAG |
| | | AAAAGCCTTACAAGTGTCCCGAGTGCGGAAAAAGCTTTAGCGATCCCGGA |
| | | CATCTGGTGAGACATCAGAGAACCCACACCGGCGAAAAGCCTTATAAATG |
| | | TCCCGAATGTGGCAAGTCCTTTAGCACCCATCTGGATCTGATTAGACACCA |
| | | AAGAACCCACACCGGCGAGAAACCCACCGGAAAAAAGACCAGCGCTAGC |
| | | GGCAGCGGCGGCGGCAGCGGCGGCGACGCCAAGAGCCTGACCGCCTGGA |
| | | GCCGGACCCTGGTGACCTTCAAGGACGTGTTCGTGGACTTCACCCGGGAG |
| | | GAGTGGAAGCTGCTGGACACCGCCCAGCAGATCCTGTACCGGAACGTGAT |
| | | GCTGGAGAACTACAAGAACCTGGTGAGCCTGGGCTACCAGCTGACCAAGC |
| | | CCGACGTGATCCTGCGGCTGGAGAAGGGCGAGGAGCCCTGGCTGGTGGAG |
| | | CGGGAGATCCACCAGGAGACCCACCCCGACAGCGAGACCGCCTTCGAGAT |
| | | CAAGAGCAGCGTGAGCGGCGGCAAGCGGCCCGCCGCCACCAAGAAGGCC |
| | | GGCCAGGCCAAGAAGAAGAAGGGCAGCTACCCCTACGACGTGCCCGACT |
| | | ACGCCTGAGCGGCCGCTTAATTAAGCTGCCTTCTGCGGGGCTTGCCTTCTG |
| | | GCCATGCCCTTCTTCTCTCCCTTGCACCTGTACCTCTTGGTCTTTGAATAAA |
| | | GCCTGAGTAGGAAGTCTAGAAAAAAAAAAAAAAAAAAAAAAAAAAAAA |
| | | AAAAAAAAAAAAAAAAAAAAAAAAAAAAAAAAAAAAAAAAAAAAAAAA |
| | | AAA |
| ZF6- | 60 | AGGAAATAAGAGAGAAAAGAAGAGTAAGAAGAAATATAAGAGCCACCAT |
| KRAB | | GGCCCCCAAGAAGAAGCGGAAGGTGGGCATCCACGGCGTGCCCGCCGCC |
| (nt) | | GGCAGCAGCGGATCCCTGGAGCCCGGCGAAAAGCCTTACAAATGTCCCGA |
| | | GTGCGGAAAGTCCTTCAGCGACCCCGGCGCTCTGGTGAGACATCAAAGAA |
| | | CACATACCGGCGAGAAACCTTATAAATGCCCCGAATGTGGAAAATCCTTC |
| | | AGCGAAAGAAGCCATCTGAGGGAACACCAGAGGACCCACACCGGCGAAA |
| | | AACCTTATAAGTGCCCCGAATGCGGAAAAAGCTTTTCTAGAAGCGATCAT |
| | | CTGACCAACCATCAGAGAACACACACCGGCGAAAAGCCCTATAAATGTCC |
| | | CGAGTGTGGCAAATCCTTTAGCGAGAGGTCCCATCTGAGAGAGCACCAGA |
| | | GGACACATACCGGAGAGAAGCCCTACAAGTGCCCCGAGTGTGGCAAGAG |
| | | CTTTAGCAGAAGCGACCATCTGACCAATCATCAAAGGACCCACACCGGAG |
| | | AGAAGCCTTACAAGTGTCCCGAGTGCGGAAAGTCCTTTTCCGATCCCGGC |
| | | CACCTCGTGAGGCACCAAAGAACCCATACCGGCGAGAAACCCTACAAATG |
| | | CCCCGAGTGTGGAAAGAGCTTCTCCAGAAGCGACAAGCTGGTGAGGCATC |
| | | AGAGGACACACACCGGCGAAAAACCCACCGGCAAGAAAACCAGCGCTAG |
| | | CGGCAGCGGCGGCGGCAGCGGCGGCGACGCCAAGAGCCTGACCGCCTGG |
| | | AGCCGGACCCTGGTGACCTTCAAGGACGTGTTCGTGGACTTCACCCGGGA |
| | | GGAGTGGAAGCTGCTGGACACCGCCCAGCAGATCCTGTACCGGAACGTGA |
| | | TGCTGGAGAACTACAAGAACCTGGTGAGCCTGGGCTACCAGCTGACCAAG |
| | | CCCGACGTGATCCTGCGGCTGGAGAAGGGCGAGGAGCCCTGGCTGGTGGA |
| | | GCGGGAGATCCACCAGGAGACCCACCCCGACAGCGAGACCGCCTTCGAG |
| | | ATCAAGAGCAGCGTGAGCGGCGGCAAGCGGCCCGCCGCCACCAAGAAGG |
| | | CCGGCCAGGCCAAGAAGAAGAAGGGCAGCTACCCCTACGACGTGCCCGA |
| | | CTACGCCTGAGCGGCCGCTTAATTAAGCTGCCTTCTGCGGGGCTTGCCTTC |
| | | TGGCCATGCCCTTCTTCTCTCCCTTGCACCTGTACCTCTTGGTCTTTGAATA |
| | | AAGCCTGAGTAGGAAGTCTAGAAAAAAAAAAAAAAAAAAAAAAAAAAA |
| | | AAAAAAAAAAAAAAAAAAAAAAAAAAAAAAAAAAAAAAAAAAAAAAAA |
| | | AAAAA |
| ZF54- | 189 | AGGAAATAAGAGAGAAAAGAAGAGTAAGAAGAAATATAAGAGCCACCAT |
| KRAB | | GGCCCCCAAGAAGAAGCGGAAGGTGGGCATCCACGGCGTGCCCGCCGCC |
| | | GGCAGCAGCGGATCCCTGGAGCCTGGAGAGAAACCCTACAAATGCCCGG |
| | | AATGCGGGAAGTCCTTTTCCGAACGCTCGCACCTGAGGGAACACCAGAGA |
| | | ACTCACACCGGCGAAAAACCCTATAAGTGCCCAGAATGCGGAAAGAGCTT |
| | | TTCACGGTCGGACAACCTCGTGCGGCACCAACGCACTCATACCGGAGAGA |
| | | AGCCGTACAAGTGTCCTGAGTGCGGAAAGTCATTCTCCGACTGCCGGGAT |
| | | TTGGCCCGCCACCAAAGAACACACACTGGCGAAAAGCCCTACAAGTGCCC |
| | | GGAGTGTGGAAAGTCCTTCAGCACTTCCGGAGAGCTGGTCCGGCACCAGA |
| | | GGACCCACACCGGGGAGAAGCCTTACAAATGTCCAGAGTGCGGTAAAAG |
| | | CTTCTCCACCACCGGCAACCTCACCGTGCACCAGCGGACCCACACTGGAG |
| | | AAAAGCCGTATAAATGCCCCGAATGCGGCAAGAGCTTCTCGCGATCCGAT |
| | | AAGCTTGTGCGGCATCAGAGAACGCACACTGGGGAAAAGCCTTATAAGTG |
| | | TCCGGAGTGCGGCAAATCCTTCTCCCGCACTGACACCCTGCGGGACCATC |
| | | AGCGCACCCATACCGGCAAAAAGACCTCTGCTAGCGGCAGCGGCGGCGG |
| | | CAGCGGCGGCGCCCGGGACGACGCCAAGAGCCTGACCGCCTGGAGCCGG |
| | | ACCCTGGTGACCTTCAAGGACGTGTTCGTGGACTTCACCCGGGAGGAGTG |
| | | GAAGCTGCTGGACACCGCCCAGCAGATCCTGTACCGGAACGTGATGCTGG |
| | | AGAACTACAAGAACCTGGTGAGCCTGGGCTACCAGCTGACCAAGCCCGAC |
| | | GTGATCCTGCGGCTGGAGAAGGGCGAGGAGCCCTGGCTGGTGGAGCGGG |
| | | AGATCCACCAGGAAACCCACCCCGACAGCGAAACCGCCTTCGAGATCAAG |
| | | AGCAGCGTGCCCAGCAGCGGCGGCAAGCGGCCCGCCGCCACCAAGAAGG |
| | | CCGGCCAGGCCAAGAAGAAGAAGGGCAGCTACCCCTACGACGTGCCCGA |
| | | CTACGCCTGAGCGGCCGCTTAATTAAGCTGCCTTCTGCGGGGCTTGCCTTC |
| | | TGGCCATGCCCTTCTTCTCTCCCTTGCACCTGTACCTCTTGGTCTTTGAATA |
| | | AAGCCTGAGTAGGAAGTCTAGAAAAAAAAAAAAAAAAAAAAAAAAAAA |
| | | AAAAAAAAAAAAAAAAAAAAAAAAAAAAAAAAAAAAAAAAAAAAAAAA |
| | | AAAAAAAAAAAAAAAAAAAAAAAAAAAAAAAAAAAA |
| ZF61- | 193 | AGGAAATAAGAGAGAAAAGAAGAGTAAGAAGAAATATAAGAGCCACCAT |
| KRAB | | GGCCCCCAAGAAGAAGCGGAAGGTGGGCATCCACGGCGTGCCCGCCGCC |
| | | GGCAGCAGCGGATCCCTTGAACCCGGGGAGAAGCCCTACAAGTGCCCGG |
| | | AATGCGGAAAGAGCTTCAGCCAGAAGTCCTCGCTGATCGCGCACCAGAGG |
| | | ACTCACACCGGCGAAAAGCCATACAAGTGTCCTGAGTGTGGCAAATCCTT |
| | | CTCGCACAAGAACGCACTGCAGAACCACCAGAGAACCCACACCGGGGAA |
| | | AAGCCGTATAAGTGCCCCGAATGTGGAAAGTCGTTCAGCCAGTCATCCAA |
| | | CCTCGTGCGCCATCAAAGGACTCATACCGGAGAGAAACCTTACAAATGCC |
| | | CTGAATGCGGCAAATCTTTCTCCCGGAATGATGCCCTGACCGAGCACCAG |
| | | CGCACTCACACGGGAGAGAAGCCGTACAAATGTCCGGAGTGCGGAAAGT |
| | | CCTTCTCCGACAAGAAGGACTTGACCAGACACCAGCGGACCCATACTGGC |
| | | GAAAAACCCTATAAGTGTCCAGAGTGCGGGAAGTCCTTTAGCCAAGCCGG |
| | | TCACCTCGCTTCCCACCAACGGACCCACACAGGAGAAAAGCCTTATAAAT |
| | | GCCCCGAGTGCGGCAAAAGCTTCTCCGATAAGAAGGACCTGACTCGGCAT |
| | | CAGCGCACCCATACCGGAAAGAAAACCTCAGCTAGCGGCAGCGGCGGCG |
| | | GCAGCGGCGGCGCCCGGGACGACGCCAAGAGCCTGACCGCCTGGAGCCG |
| | | GACCCTGGTGACCTTCAAGGACGTGTTCGTGGACTTCACCCGGGAGGAGT |
| | | GGAAGCTGCTGGACACCGCCCAGCAGATCCTGTACCGGAACGTGATGCTG |
| | | GAGAACTACAAGAACCTGGTGAGCCTGGGCTACCAGCTGACCAAGCCCGA |
| | | CGTGATCCTGCGGCTGGAGAAGGGCGAGGAGCCCTGGCTGGTGGAGCGG |
| | | GAGATCCACCAGGAAACCCACCCCGACAGCGAAACCGCCTTCGAGATCAA |
| | | GAGCAGCGTGCCCAGCAGCGGCGGCAAGCGGCCCGCCGCCACCAAGAAG |
| | | GCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTT |
| | | CTGGCCATGCCCTTCTTCTCTCCCTTGCACCTGTACCTCTTGGTCTTTGAAT |
| | | AAAGCCTGAGTAGGAAGTCTAGAAAAAAAAAAAAAAAAAAAAAAAAAA |
| | | AAAAAAAAAAAAAAAAAAAAAAAAAAAAAAAAAAAAAAAAAAAAAAAA |
| | | AAAAAAAAAAAAAAAAAAAAAAAAAAAAAAAAAAAAA |
| ZF67- | 194 | AGGAAATAAGAGAGAAAAGAAGAGTAAGAAGAAATATAAGAGCCACCAT |
| KRAB | | GGCCCCCAAGAAGAAGCGGAAGGTGGGCATCCACGGCGTGCCCGCCGCC |
| | | GGCAGCAGCGGATCCCTGGAGCCTGGCGAAAAACCCTATAAGTGCCCAGA |
| | | ATGCGGAAAGAGCTTTTCACGGTCGGACAACCTCGTGCGGCACCAACGCA |
| | | CTCATACCGGAGAGAAGCCGTACAAGTGTCCTGAGTGCGGAAAGTCATTC |
| | | TCCGACTGCCGGGATTTGGCCCGCCACCAAAGAACACACACTGGCGAAAA |
| | | GCCCTACAAGTGCCCGGAGTGTGGAAAGTCCTTCAGCACTTCCGGAGAGC |
| | | TGGTCCGGCACCAGAGGACCCACACCGGGGAGAAGCCTTACAAATGTCCA |
| | | GAGTGCGGTAAAAGCTTCTCCACCACCGGCAACCTCACCGTGCACCAGCG |
| | | GACCCACACTGGAGAAAAGCCGTATAAATGCCCCGAATGCGGCAAGAGCT |
| | | TCTCGCGATCCGATAAGCTTGTGCGGCATCAGAGAACGCACACTGGGGAA |
| | | AAGCCTTATAAGTGTCCGGAGTGCGGCAAATCCTTCTCCCGCACTGACACC |
| | | CTGCGGGACCACCAGAGAACCCATACTGGCGAGAAGCCATACAAATGCCC |
| | | GGAATGTGGAAAGAGTTTCTCGCGCGAGGACAACCTCCACACCCATCAGC |
| | | GCACCCATACCGGCAAAAAGACCTCTGCTAGCGGCAGCGGCGGCGGCAG |
| | | CGGCGGCGCCCGGGACGACGCCAAGAGCCTGACCGCCTGGAGCCGGACC |
| | | CTGGTGACCTTCAAGGACGTGTTCGTGGACTTCACCCGGGAGGAGTGGAA |
| | | GCTGCTGGACACCGCCCAGCAGATCCTGTACCGGAACGTGATGCTGGAGA |
| | | ACTACAAGAACCTGGTGAGCCTGGGCTACCAGCTGACCAAGCCCGACGTG |
| | | ATCCTGCGGCTGGAGAAGGGCGAGGAGCCCTGGCTGGTGGAGCGGGAGA |
| | | TCCACCAGGAAACCCACCCCGACAGCGAAACCGCCTTCGAGATCAAGAGC |
| | | AGCGTGCCCAGCAGCGGCGGCAAGCGGCCCGCCGCCACCAAGAAGGCCG |
| | | GCCAGGCCAAGAAGAAGAAGGGCAGCTACCCCTACGACGTGCCCGACTA |
| | | CGCCTGAGCGGCCGCTTAATTAAGCTGCCTTCTGCGGGGCTTGCCTTCTGG |
| | | CCATGCCCTTCTTCTCTCCCTTGCACCTGTACCTCTTGGTCTTTGAATAAAG |
| | | CCTGAGTAGGAAGTCTAGAAAAAAAAAAAAAAAAAAAAAAAAAAAAAA |
| | | AAAAAAAAAAAAAAAAAAAAAAAAAAAAAAAAAAAAAAAAAAAAAAAA |
| | | AAAAAAAAAAAAAAAAAAAAAAAAAAAAAAAAA |
| ZF68- | 195 | AGGAAATAAGAGAGAAAAGAAGAGTAAGAAGAAATATAAGAGCCACCAT |
| KRAB | | GGCCCCCAAGAAGAAGCGGAAGGTGGGCATCCACGGCGTGCCCGCCGCC |
| | | GGCAGCAGCGGATCCCTGGAACCCGGAGAGAAACCCTACAAATGCCCAG |
| | | AGTGCGGCAAATCGTTCTCACGGTCCGATCACCTCACCACCCACCAGAGG |
| | | ACCCATACCGGGGAGAAGCCTTACAAGTGTCCTGAGTGTGGAAAGTCCTT |
| | | CAGCCAAAAGTCCTCGCTGATCGCACACCAGCGCACGCACACTGGGGAAA |
| | | AGCCATATAAATGCCCGGAGTGTGGCAAATCCTTCTCCCGCCGCGACGAA |
| | | CTGAACGTGCACCAGAGAACCCACACTGGAGAGAAGCCGTATAAGTGTCC |
| | | GGAGTGCGGAAAGAGCTTCTCGCAATCCGGGGACCTTCGGAGACATCAGA |
| | | GGACACACACTGGCGAAAAGCCCTATAAGTGCCCTGAGTGCGGGAAGTCC |
| | | TTTAGCCAAGCCGGTCACCTGGCCTCCCACCAACGGACTCACACCGGCGA |
| | | AAAACCGTACAAGTGCCCCGAATGCGGAAAGTCGTTCTCTACCTCCGGAA |
| | | ACTTGACCGAACACCAGCGGACCCACACCGGAGAAAAGCCGTACAAATG |
| | | TCCTGAATGCGGCAAAAGCTTCAGCCAGGCCGGTCATCTCGCGAGCCATC |
| | | AGCGGACTCATACTGGCAAAAAGACCTCAGCTAGCGGCAGCGGCGGCGG |
| | | CAGCGGCGGCGCCCGGGACGACGCCAAGAGCCTGACCGCCTGGAGCCGG |
| | | ACCCTGGTGACCTTCAAGGACGTGTTCGTGGACTTCACCCGGGAGGAGTG |
| | | GAAGCTGCTGGACACCGCCCAGCAGATCCTGTACCGGAACGTGATGCTGG |
| | | AGAACTACAAGAACCTGGTGAGCCTGGGCTACCAGCTGACCAAGCCCGAC |
| | | GTGATCCTGCGGCTGGAGAAGGGCGAGGAGCCCTGGCTGGTGGAGCGGG |
| | | AGATCCACCAGGAAACCCACCCCGACAGCGAAACCGCCTTCGAGATCAAG |
| | | AGCAGCGTGCCCAGCAGCGGCGGCAAGCGGCCCGCCGCCACCAAGAAGG |
| | | CCGGCCAGGCCAAGAAGAAGAAGGGCAGCTACCCCTACGACGTGCCCGA |
| | | CTACGCCTGAGCGGCCGCTTAATTAAGCTGCCTTCTGCGGGGCTTGCCTTC |
| | | TGGCCATGCCCTTCTTCTCTCCCTTGCACCTGTACCTCTTGGTCTTTGAATA |
| | | AAGCCTGAGTAGGAAGTCTAGAAAAAAAAAAAAAAAAAAAAAAAAAAA |
| | | AAAAAAAAAAAAAAAAAAAAAAAAAAAAAAAAAAAAAAAAAAAAAAAA |
| | | AAAAAAAAAAAAAAAAAAAAAAAAAAAAAAAAAAAA |

In some embodiments, an expression repressor comprises a targeting moiety comprising a Zn Finger domain (e.g., having an amino acid sequence according to any of SEQ ID NO: 5-10 or 169-172), and an effector moiety comprising KRAB (e.g., an amino acid sequence SEQ ID NO: 18), e.g., a KRAB domain. In some embodiments, an expression repressor described herein comprises an amino sequence of any of SEQ ID NOs: 22, 23, 24, 25, 26, 27, 139-144, 177-180, or 183-186. The protein sequence of these exemplary expression repressors are disclosed in Table 7. In some embodiments, an expression repressor described herein comprises an amino acid sequence of any of SEQ ID NOs: 22-27, 139-144, 177-180, 183-186 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

TABLE 7
Amino Acid sequences of exemplary Zinc Finger-KRAB effectors

| | SEQ | |
|---|---|---|
| | ID | |
| NAME | NO. | SEQUENCE |
| ZF1- | 22 | MAPKKKRKVGIHGVPAAGSSGSLEPGEKPYKCPECGKSFSRSDKLTEHQRTHTGEKPYKCPECGKSFSTKN |
| KRAB | | SLTEHQRTHTGEKPYKCPECGKSFSQSGDLRRHQRTHTGEKPYKCPECGKSFSTTGALTEHQRTHTGEKPY |
| (aa) | | KCPECGKSFSDSGNLRVHQRTHTGEKPYKCPECGKSFSQRAHLERHQRTHTGEKPYKCPECGKSFSTSGEL |
| | | VRHQRTHTGEKPTGKKTSASGSGGGSGGDAKSLTAWSRTLVTFKDVFVDFTREEWKLLDTAQQILYRNV |
| | | MLENYKNLVSLGYQLTKPDVILRLEKGEEPWLVEREIHQETHPDSETAFEIKSSVSGGKRPAATKKAGQAK |
| | | KKKGSYPYDVPDYA |
| ZF2- | 23 | MAPKKKRKVGIHGVPAAGSSGSLEPGEKPYKCPECGKSFSSPADLTRHQRTHTGEKPYKCPECGKSFSDPG |
| KRAB | | HLVRHQRTHTGEKPYKCPECGKSFSRSDNLVRHQRTHTGEKPYKCPECGKSFSQSSSLVRHQRTHTGEKP |
| (aa) | | YKCPECGKSFSTSHSLTEHQRTHTGEKPYKCPECGKSFSRNDALTEHQRTHTGEKPYKCPECGKSFSRNDA |
| | | LTEHQRTHTGEKPTGKKTSASGSGGGSGGDAKSLTAWSRTLVTFKDVFVDFTREEWKLLDTAQQILYRNV |
| | | MLENYKNLVSLGYQLTKPDVILRLEKGEEPWLVEREIHQETHPDSETAFEIKSSVSGGKRPAATKKAGQAK |
| | | KKKGSYPYDVPDYA |
| ZF3- | 24 | MAPKKKRKVGIHGVPAAGSSGSLEPGEKPYKCPECGKSFSRSDKLVRHQRTHTGEKPYKCPECGKSFSQR |
| KRAB | | AHLERHQRTHTGEKPYKCPECGKSFSDPGHLVRHQRTHTGEKPYKCPECGKSFSRSDKLVRHQRTHTGEK |
| (aa) | | PYKCPECGKSFSQLAHLRAHQRTHTGEKPYKCPECGKSFSRADNLTEHQRTHTGEKPYKCPECGKSFSDCR |
| | | DLARHQRTHTGEKPTGKKTSASGSGGGSGGDAKSLTAWSRTLVTFKDVFVDFTREEWKLLDTAQQILYR |
| | | NVMLENYKNLVSLGYQLTKPDVILRLEKGEEPWLVEREIHQETHPDSETAFEIKSSVSGGKRPAATKKAGQ |
| | | AKKKKGSYPYDVPDYA |
| ZF4- | 25 | MAPKKKRKVGIHGVPAAGSSGSLEPGEKPYKCPECGKSFSHTGHLLEHQRTHTGEKPYKCPECGKSFSTTG |
| KRAB | | NLTVHQRTHTGEKPYKCPECGKSFSDKKDLTRHQRTHTGEKPYKCPECGKSFSQSGDLRRHQRTHTGEKP |
| (aa) | | YKCPECGKSFSQRAHLERHQRTHTGEKPYKCPECGKSFSRSDKLTEHQRTHTGEKPYKCPECGKSFSRTDT |
| | | LRDHQRTHTGEKPTGKKTSASGSGGGSGGDAKSLTAWSRTLVTFKDVFVDFTREEWKLLDTAQQILYRN |
| | | VMLENYKNLVSLGYQLTKPDVILRLEKGEEPWLVEREIHQETHPDSETAFEIKSSVSGGKRPAATKKAGQA |
| | | KKKKGSYPYDVPDYA |
| ZF5- | 26 | MAPKKKRKVGIHGVPAAGSSGSLEPGEKPYKCPECGKSFSHTGHLLEHQRTHTGEKPYKCPECGKSFSTSG |
| KRAB | | NLTEHQRTHTGEKPYKCPECGKSFSTSGNLVRHQRTHTGEKPYKCPECGKSFSQLAHLRAHQRTHTGEKP |
| (aa) | | YKCPECGKSFSERSHLREHQRTHTGEKPYKCPECGKSFSDPGHLVRHQRTHTGEKPYKCPECGKSFSTHLD |
| | | LIRHQRTHTGEKPTGKKTSASGSGGGSGGDAKSLTAWSRTLVTFKDVFVDFTREEWKLLDTAQQILYRNV |
| | | MLENYKNLVSLGYQLTKPDVILRLEKGEEPWLVEREIHQETHPDSETAFEIKSSVSGGKRPAATKKAGQAK |
| | | KKKGSYPYDVPDYA |
| ZF6- | 27 | MAPKKKRKVGIHGVPAAGSSGSLEPGEKPYKCPECGKSFSDPGALVRHQRTHTGEKPYKCPECGKSFSE |
| KRAB | | RSHLREHQRTHTGEKPYKCPECGKSFSRSDHLTNHQRTHTGEKPYKCPECGKSFSERSHLREHQRTHTGE |
| (aa) | | KPYKCPECGKSFSRSDHLTNHQRTHTGEKPYKCPECGKSFSDPGHLVRHQRTHTGEKPYKCPECGKSFSR |
| | | SDKLVRHQRTHTGEKPTGKKTSASGSGGGSGGDAKSLTAWSRTLVTFKDVFVDFTREEWKLLDTAQQIL |
| | | YRNVMLENYKNLVSLGYQLTKPDVILRLEKGEEPWLVEREIHQETHPDSETAFEIKSSVSGGKRPAATKK |
| | | AGQAKKKKGSYPYDVPDYA |
| tPT2A | 134 | PLEGSSGSGSPKKKRKVGIHGVPAAGSSGSLEPGEKPYKCPECGKSFSRSDKLVRHQRTHTGEKPYKCPE |
| fragment | | CGKSFSQRAHLERHQRTHTGEKPYKCPECGKSFSDPGHLVRHQRTHTGEKPYKCPECGKSFSRSDKLVR |
| 3 + ZF3- | | HQRTHTGEKPYKCPECGKSFSQLAHLRAHQRTHTGEKPYKCPECGKSFSRADNLTEHQRTHTGEKPYKC |
| KRAB | | PECGKSFSDCRDLARHQRTHTGEKPTGKKTSASGSGGGSGGDAKSLTAWSRTLVTFKDVFVDFTREEWK |
| | | LLDTAQQILYRNVMLENYKNLVSLGYQLTKPDVILRLEKGEEPWLVEREIHQETHPDSETAFEIKSSVSGG |
| | | KRPAATKKAGQAKKKKGS |
| ZF1- | 139 | MAPKKKRKVGIHGVPAAGSSGSLEPGEKPYKCPECGKSFSRSDKLTEHQRTHTGEKPYKCPECGKSFSTK |
| KRAB | | NSLTEHQRTHTGEKPYKCPECGKSFSQSGDLRRHQRTHTGEKPYKCPECGKSFSTTGALTEHQRTHTGEK |
| without | | PYKCPECGKSFSDSGNLRVHQRTHTGEKPYKCPECGKSFSQRAHLERHQRTHTGEKPYKCPECGKSFSTS |
| HA | | GELVRHQRTHTGEKPTGKKTSASGSGGGSGGDAKSLTAWSRTLVTFKDVFVDFTREEWKLLDTAQQILY |
| (aa) | | RNVMLENYKNLVSLGYQLTKPDVILRLEKGEEPWLVEREIHQETHPDSETAFEIKSSVSGGKRPAATKKA |
| | | GQAKKKKGS |
| ZF2- | 140 | MAPKKKRKVGIHGVPAAGSSGSLEPGEKPYKCPECGKSFSSPADLTRHQRTHTGEKPYKCPECGKSFSDP |
| KRAB | | GHLVRHQRTHTGEKPYKCPECGKSFSRSDNLVRHQRTHTGEKPYKCPECGKSFSQSSSLVRHQRTHTGE |
| without | | KPYKCPECGKSFSTSHSLTEHQRTHTGEKPYKCPECGKSFSRNDALTEHQRTHTGEKPYKCPECGKSFSR |
| HA | | NDALTEHQRTHTGEKPTGKKTSASGSGGGSGGDAKSLTAWSRTLVTFKDVFVDFTREEWKLLDTAQQIL |
| (aa) | | YRNVMLENYKNLVSLGYQLTKPDVILRLEKGEEPWLVEREIHQETHPDSETAFEIKSSVSGGKRPAATKK |
| | | AGQAKKKKGS |
| ZF3- | 141 | MAPKKKRKVGIHGVPAAGSSGSLEPGEKPYKCPECGKSFSRSDKLVRHQRTHTGEKPYKCPECGKSFSQ |
| KRAB | | RAHLERHQRTHTGEKPYKCPECGKSFSDPGHLVRHQRTHTGEKPYKCPECGKSFSRSDKLVRHQRTHTG |
| without | | EKPYKCPECGKSFSQLAHLRAHQRTHTGEKPYKCPECGKSFSRADNLTEHQRTHTGEKPYKCPECGKSFS |
| HA | | DCRDLARHQRTHTGEKPTGKKTSASGSGGGSGGDAKSLTAWSRTLVTFKDVFVDFTREEWKLLDTAQQ |
| (aa) | | ILYRNVMLENYKNLVSLGYQLTKPDVILRLEKGEEPWLVEREIHQETHPDSETAFEIKSSVSGGKRPAATK |
| | | KAGQAKKKKGS |
| ZF4- | 142 | MAPKKKRKVGIHGVPAAGSSGSLEPGEKPYKCPECGKSFSHTGHLLEHQRTHTGEKPYKCPECGKSFSTT |
| KRAB | | GNLTVHQRTHTGEKPYKCPECGKSFSDKKDLTRHQRTHTGEKPYKCPECGKSFSQSGDLRRHQRTHTGE |
| without | | KPYKCPECGKSFSQRAHLERHQRTHTGEKPYKCPECGKSFSRSDKLTEHQRTHTGEKPYKCPECGKSFSR |
| HA | | TDTLRDHQRTHTGEKPTGKKTSASGSGGGSGGDAKSLTAWSRTLVTFKDVFVDFTREEWKLLDTAQQIL |
| (aa) | | YRNVMLENYKNLVSLGYQLTKPDVILRLEKGEEPWLVEREIHQETHPDSETAFEIKSSVSGGKRPAATKK |
| | | AGQAKKKKGS |
| ZF5- | 143 | MAPKKKRKVGIHGVPAAGSSGSLEPGEKPYKCPECGKSFSHTGHLLEHQRTHTGEKPYKCPECGKSFSTS |
| KRAB | | GNLTEHQRTHTGEKPYKCPECGKSFSTSGNLVRHQRTHTGEKPYKCPECGKSFSQLAHLRAHQRTHTGE |
| without | | KPYKCPECGKSFSERSHLREHQRTHTGEKPYKCPECGKSFSDPGHLVRHQRTHTGEKPYKCPECGKSFST |
| HA | | HLDLIRHQRTHTGEKPTGKKTSASGSGGGSGGDAKSLTAWSRTLVTFKDVFVDFTREEWKLLDTAQQIL |
| (aa) | | YRNVMLENYKNLVSLGYQLTKPDVILRLEKGEEPWLVEREIHQETHPDSETAFEIKSSVSGGKRPAATKK |
| | | AGQAKKKKGS |
| ZF6- | 144 | MAPKKKRKVGIHGVPAAGSSGSLEPGEKPYKCPECGKSFSDPGALVRHQRTHTGEKPYKCPECGKSFSE |
| KRAB | | RSHLREHQRTHTGEKPYKCPECGKSFSRSDHLTNHQRTHTGEKPYKCPECGKSFSERSHLREHQRTHTGE |
| without | | KPYKCPECGKSFSRSDHLTNHQRTHTGEKPYKCPECGKSFSDPGHLVRHQRTHTGEKPYKCPECGKSFSR |
| HA | | SDKLVRHQRTHTGEKPTGKKTSASGSGGGSGGDAKSLTAWSRTLVTFKDVFVDFTREEWKLLDTAQQIL |
| (aa) | | YRNVMLENYKNLVSLGYQLTKPDVILRLEKGEEPWLVEREIHQETHPDSETAFEIKSSVSGGKRPAATKK |
| | | AGQAKKKKGS |
| ZF54- | 177 | MAPKKKRKVGIHGVPAAGSSGSLEPGEKPYKCPECGKSFSERSHLREHQRTHTGEKPYKCPECGKSFSRS |
| KRAB aa | | DNLVRHQRTHTGEKPYKCPECGKSFSDCRDLARHQRTHTGEKPYKCPECGKSFSTSGELVRHQRTHTGE |
| | | KPYKCPECGKSFSTTGNLTVHQRTHTGEKPYKCPECGKSFSRSDKLVRHQRTHTGEKPYKCPECGKSFSR |
| | | TDTLRDHQRTHTGKKTSASGSGGGSGGARDDAKSLTAWSRTLVTFKDVFVDFTREEWKLLDTAQQILY |
| | | RNVMLENYKNLVSLGYQLTKPDVILRLEKGEEPWLVEREIHQETHPDSETAFEIKSSVPSSGGKRPAATK |
| | | KAGQAKKKKGSYPYDVPDYA |
| ZF61- | 178 | MAPKKKRKVGIHGVPAAGSSGSLEPGEKPYKCPECGKSFSQKSSLIAHQRTHTGEKPYKCPECGKSFSHK |
| KRAB aa | | NALQNHQRTHTGEKPYKCPECGKSFSQSSNLVRHQRTHTGEKPYKCPECGKSFSRNDALTEHQRTHTGE |
| | | KPYKCPECGKSFSDKKDLTRHQRTHTGEKPYKCPECGKSFSQAGHLASHQRTHTGEKPYKCPECGKSFS |
| | | DKKDLTRHQRTHTGKKTSASGSGGGSGGARDDAKSLTAWSRTLVTFKDVFVDFTREEWKLLDTAQQIL |
| | | YRNVMLENYKNIVSIGYQITKPDVIIRLEKGEEPWIVEREIHQETHPDSETAFEIKSSVPSSGGKRPAAT |
| | | KKAGQAKKKKGSYPYDVPDYA |
| ZF67- | 179 | MAPKKKRKVGIHGVPAAGSSGSLEPGEKPYKCPECGKSFSRSDNLVRHQRTHTGEKPYKCPECGKSFSD |
| KRAB aa | | CRDLARHQRTHTGEKPYKCPECGKSFSTSGELVRHQRTHTGEKPYKCPECGKSFSTTGNLTVHQRTHTG |
| | | EKPYKCPECGKSFSRSDKLVRHQRTHTGEKPYKCPECGKSFSRTDTLRDHQRTHTGEKPYKCPECGKSFS |
| | | REDNLHTHQRTHTGKKTSASGSGGGSGGARDDAKSLTAWSRTLVTFKDVFVDFTREEWKLLDTAQQIL |
| | | YRNVMLENYKNLVSLGYQLTKPDVILRLEKGEEPWLVEREIHQETHPDSETAFEIKSSVPSSGGKRPAAT |
| | | KKAGQAKKKKGSYPYDVPDYA |
| ZF68- | 180 | MAPKKKRKVGIHGVPAAGSSGSLEPGEKPYKCPECGKSFSRSDHLTTHQRTHTGEKPYKCPECGKSFSQK |
| KRAB aa | | SSLIAHQRTHTGEKPYKCPECGKSFSRRDELNVHQRTHTGEKPYKCPECGKSFSQSGDLRRHQRTHTGEK |
| | | PYKCPECGKSFSQAGHLASHQRTHTGEKPYKCPECGKSFSTSGNLTEHQRTHTGEKPYKCPECGKSFSQA |
| | | GHLASHQRTHTGKKTSASGSGGGSGGARDDAKSLTAWSRTLVTFKDVFVDFTREEWKLLDTAQQILYR |
| | | NVMLENYKNLVSLGYQLTKPDVILRLEKGEEPWLVEREIHQETHPDSETAFEIKSSVPSSGGKRPAATKK |
| | | AGQAKKKKGSYPYDVPDYA |
| ZF54- | 183 | MAPKKKRKVGIHGVPAAGSSGSLEPGEKPYKCPECGKSFSERSHLREHQRTHTGEKPYKCPECGKSFSRS |
| KRAB aa | | DNLVRHQRTHTGEKPYKCPECGKSFSDCRDLARHQRTHTGEKPYKCPECGKSFSTSGELVRHQRTHTGE |
| without | | KPYKCPECGKSFSTTGNLTVHQRTHTGEKPYKCPECGKSFSRSDKLVRHQRTHTGEKPYKCPECGKSFSR |
| HA tag | | TDTLRDHQRTHTGKKTSASGSGGGSGGARDDAKSLTAWSRTLVTFKDVFVDFTREEWKLLDTAQQILY |
| | | RNVMLENYKNLVSLGYQLTKPDVILRLEKGEEPWLVEREIHQETHPDSETAFEIKSSVPSSGGKRPAATK |
| | | KAGQAKKKKGS |
| ZF61- | 184 | MAPKKKRKVGIHGVPAAGSSGSLEPGEKPYKCPECGKSFSQKSSLIAHQRTHTGEKPYKCPECGKSFSHK |
| KRAB aa | | NALQNHQRTHTGEKPYKCPECGKSFSQSSNLVRHQRTHTGEKPYKCPECGKSFSRNDALTEHQRTHTGE |
| without | | KPYKCPECGKSFSDKKDLTRHQRTHTGEKPYKCPECGKSFSQAGHLASHQRTHTGEKPYKCPECGKSFS |
| HA tag | | DKKDLTRHQRTHTGKKTSASGSGGGSGGARDDAKSLTAWSRTLVTFKDVFVDFTREEWKLLDTAQQIL |
| | | YRNVMLENYKNLVSLGYQLTKPDVILRLEKGEEPWLVEREIHQETHPDSETAFEIKSSVPSSGGKRPAAT |
| | | KKAGQAKKKKGS |
| ZF67- | 185 | MAPKKKRKVGIHGVPAAGSSGSLEPGEKPYKCPECGKSFSRSDNLVRHQRTHTGEKPYKCPECGKSFSD |
| KRAB aa | | CRDLARHQRTHTGEKPYKCPECGKSFSTSGELVRHQRTHTGEKPYKCPECGKSFSTTGNLTVHQRTHTG |
| without | | EKPYKCPECGKSFSRSDKLVRHQRTHTGEKPYKCPECGKSFSRTDTLRDHQRTHTGEKPYKCPECGKSFS |
| HA tag | | REDNLHTHQRTHTGKKTSASGSGGGSGGARDDAKSLTAWSRTLVTFKDVFVDFTREEWKLLDTAQQIL |
| | | YRNVMLENYKNLVSLGYQLTKPDVILRLEKGEEPWLVEREIHQETHPDSETAFEIKSSVPSSGGKRPAAT |
| | | KKAGQAKKKKGS |
| ZF68- | 186 | MAPKKKRKVGIHGVPAAGSSGSLEPGEKPYKCPECGKSFSRSDHLTTHQRTHTGEKPYKCPECGKSFSQK |
| KRAB aa | | SSLIAHQRTHTGEKPYKCPECGKSFSRRDELNVHQRTHTGEKPYKCPECGKSFSQSGDLRRHQRTHTGEK |
| without | | PYKCPECGKSFSQAGHLASHQRTHTGEKPYKCPECGKSFSTSGNLTEHQRTHTGEKPYKCPECGKSFSQA |
| HA tag | | GHLASHQRTHTGKKTSASGSGGGSGGARDDAKSLTAWSRTLVTFKDVFVDFTREEWKLLDTAQQILYR |
| | | NVMLENYKNLVSLGYQLTKPDVILRLEKGEEPWLVEREIHQETHPDSETAFEIKSSVPSSGGKRPAATKK |
| | | AGQAKKKKGS |

In some embodiments, an expression repressor comprises a targeting moiety comprising a Zn Finger domain (e.g., one encoded by a nucleotide sequence of any of SEQ ID NO: 44-49 or 115), and an effector moiety comprising MQ1, e.g., a bacterial MQ1 (e.g., one encoded by a nucleotide sequence of SEQ ID NO: 52). In some embodiments, the expression repressors are encoded by the nucleic sequence of SEQ ID NOs: 61, 62, 63, 64, 65, 66, 116, 117, 118, or 130. The nucleic acid sequence of these exemplary expression repressors are disclosed in Table 8. In some embodiments, a nucleic acid described herein comprises a nucleic acid sequence of any of SEQ ID NO: 61-66, 116-118, 130 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the nucleic acid sequence comprises a poly-A sequence, and in other embodiments, the nucleic acid lacks the poly-A sequence. For example, in some embodiments, a nucleic acid described herein comprises a sequence according to any of SEQ ID NO: 61-66, 116-118, or 130 (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the 3′ poly-A sequence, or comprising a 3′ poly-A sequence of a shorter length.

TABLE 8
Nucleotide sequences of exemplary ZF-MQ1 effectors

| | SEQ | |
|---|---|---|
| | ID | |
| NAME | NO | SEQUENCE |
| ZF7- | 61 | AGGAAATAAGAGAGAAAAGAAGAGTAAGAAGAAATATAAGAGCCACCAT |
| MQ1 | | GGCCCCCAAGAAGAAGCGGAAGGTGGGCATCCACGGCGTGCCCGCCGCC |
| (nt) | | GGCAGCAGCGGATCCCTGGAGCCCGGAGAGAAGCCCTACAAATGCCCCG |
| | | AGTGTGGAAAGAGCTTCTCTAGAAATGACGCTCTGACAGAACACCAGAGG |
| | | ACCCATACCGGCGAGAAACCTTACAAATGCCCCGAGTGCGGAAAAAGCTT |
| | | TAGCGATTGCAGAGATCTGGCTAGACATCAGAGAACACACACCGGCGAGA |
| | | AGCCCTATAAGTGCCCCGAATGCGGCAAGAGCTTTAGCGACCCCGGCCAT |
| | | CTGGTGAGACATCAAAGGACACATACCGGAGAAAAACCTTACAAGTGCCC |
| | | CGAGTGCGGAAAGTCCTTCTCCCAGAGCGGCCATCTCACCGAGCATCAAA |
| | | GGACCCACACCGGCGAAAAGCCTTATAAATGTCCCGAATGTGGCAAGTCC |
| | | TTCTCTAGAGAGGATAATCTGCACACCCATCAGAGGACCCACACCGGCGA |
| | | AAAGCCTTATAAATGCCCCGAATGTGGAAAGTCCTTTTCCACCAAGAACT |
| | | CTCTGACCGAGCATCAGAGGACACACACCGGAGAGAAACCCTATAAATGT |
| | | CCCGAGTGTGGCAAGAGCTTCAGCAGAGCCGACAATCTGACAGAGCACCA |
| | | AAGAACACATACCGGCGAAAAGCCCACCGGCAAAAAGACCAGCGCTAGC |
| | | GGCAGCGGCGGCGGCAGCGGCGGCGCCCGGGACAGCAAGGTGGAGAACA |
| | | AGACCAAGAAGCTGCGGGTGTTCGAGGCCTTCGCCGGCATCGGCGCCCAG |
| | | CGGAAGGCCCTGGAGAAGGTGCGGAAGGACGAGTACGAGATCGTGGGCC |
| | | TGGCCGAGTGGTACGTGCCCGCCATCGTGATGTACCAGGCCATCCACAAC |
| | | AACTTCCACACCAAGCTGGAGTACAAGAGCGTGAGCCGGGAGGAGATGA |
| | | TCGACTACCTGGAGAACAAGACCCTGAGCTGGAACAGCAAGAACCCCGTG |
| | | AGCAACGGCTACTGGAAGCGGAAGAAGGACGACGAGCTGAAGATCATCT |
| | | ACAACGCCATCAAGCTGAGCGAGAAGGAGGGCAACATCTTCGACATCCGG |
| | | GACCTGTACAAGCGGACCCTGAAGAACATCGACCTGCTGACCTACAGCTT |
| | | CCCCTGCCAGGACCTGAGCCAGCAGGGCATCCAGAAGGGCATGAAGCGG |
| | | GGCAGCGGCACCCGGAGCGGCCTGCTGTGGGAGATCGAGCGGGCCCTGG |
| | | ACAGCACCGAGAAGAACGACCTGCCCAAGTACCTGCTGATGGAGAACGTG |
| | | GGCGCCCTGCTGCACAAGAAGAACGAGGAGGAGCTGAACCAGTGGAAGC |
| | | AGAAGCTGGAGAGCCTGGGCTACCAGAACAGCATCGAGGTGCTGAACGC |
| | | CGCCGACTTCGGCAGCAGCCAGGCCCGGCGGCGGGTGTTCATGATCAGCA |
| | | CCCTGAACGAGTTCGTGGAGCTGCCCAAGGGCGACAAGAAGCCCAAGAG |
| | | CATCAAGAAGGTGCTGAACAAGATCGTGAGCGAGAAGGACATCCTGAAC |
| | | AACCTGCTGAAGTACAACCTGACCGAGTTCAAGAAAACCAAGAGCAACAT |
| | | CAACAAGGCCAGCCTGATCGGCTACAGCAAGTTCAACAGCGAGGGCTACG |
| | | TGTACGACCCCGAGTTCACCGGCCCCACCCTGACCGCCAGCGGCGCCAAC |
| | | AGCCGGATCAAGATCAAGGACGGCAGCAACATCCGGAAGATGAACAGCG |
| | | ACGAGACCTTCCTGTACATCGGCTTCGACAGCCAGGACGGCAAGCGGGTG |
| | | AACGAGATCGAGTTCCTGACCGAGAACCAGAAGATCTTCGTGTGCGGCAA |
| | | CAGCATCAGCGTGGAGGTGCTGGAGGCCATCATCGACAAGATCGGCGGCC |
| | | CCAGCAGCGGCGGCAAGCGGCCCGCCGCCACCAAGAAGGCCGGCCAGGC |
| | | CAAGAAGAAGAAGGGCAGCTACCCCTACGACGTGCCCGACTACGCCTGA |
| | | GCGGCCGCTTAATTAAGCTGCCTTCTGCGGGGCTTGCCTTCTGGCCATGCC |
| | | CTTCTTCTCTCCCTTGCACCTGTACCTCTTGGTCTTTGAATAAAGCCTGAGT |
| | | AGGAAGTCTAGAAAAAAAAAAAAAAAAAAAAAAAAAAAAAAAAAAAAA |
| | | AAAAAAAAAAAAAAAAAAAAAAAAAAAAAAAAAAAAAAAAAAA |
| ZF8- | 62 | AGGAAATAAGAGAGAAAAGAAGAGTAAGAAGAAATATAAGAGCCACCAT |
| MQ1 | | GGCCCCCAAGAAGAAGCGGAAGGTGGGCATCCACGGCGTGCCCGCCGCC |
| (nt) | | GGCAGCAGCGGATCCCTGGAGCCCGGCGAGAAACCCTACAAGTGCCCCG |
| | | AGTGTGGCAAATCCTTCTCTAGATCCGACAAACTGACCGAACATCAGAGG |
| | | ACCCATACCGGCGAAAAACCTTATAAATGTCCCGAGTGCGGAAAGTCCTT |
| | | CTCTAGAAGGGACGAGCTGAACGTGCATCAGAGAACACATACCGGCGAG |
| | | AAGCCCTATAAATGCCCCGAATGCGGCAAAAGCTTCTCTAGAAGCGATCA |
| | | TCTGACCAACCACCAGAGAACCCATACCGGAGAAAAGCCTTACAAGTGTC |
| | | CCGAATGTGGAAAATCCTTCAGCTCCCCCGCCGATCTGACCAGACACCAA |
| | | AGGACCCACACCGGCGAGAAGCCCTATAAATGCCCCGAGTGCGGCAAGA |
| | | GCTTTTCCAGATCCGACCATCTGACCAATCATCAAAGAACCCACACCGGC |
| | | GAAAAGCCTTATAAATGTCCCGAGTGCGGCAAATCCTTTTCCAGCAAGAA |
| | | GGCTCTGACCGAGCATCAAAGGACCCATACCGGCGAGAAGCCTTACAAAT |
| | | GCCCCGAGTGTGGAAAGTCCTTTAGCACCCATCTGGATCTGATTAGACACC |
| | | AGAGGACACACACCGGAGAGAAACCCACCGGCAAAAAGACCAGCGCTAG |
| | | CGGCAGCGGCGGCGGCAGCGGCGGCGCCCGGGACAGCAAGGTGGAGAAC |
| | | AAGACCAAGAAGCTGCGGGTGTTCGAGGCCTTCGCCGGCATCGGCGCCCA |
| | | GCGGAAGGCCCTGGAGAAGGTGCGGAAGGACGAGTACGAGATCGTGGGC |
| | | CTGGCCGAGTGGTACGTGCCCGCCATCGTGATGTACCAGGCCATCCACAA |
| | | CAACTTCCACACCAAGCTGGAGTACAAGAGCGTGAGCCGGGAGGAGATG |
| | | ATCGACTACCTGGAGAACAAGACCCTGAGCTGGAACAGCAAGAACCCCGT |
| | | GAGCAACGGCTACTGGAAGCGGAAGAAGGACGACGAGCTGAAGATCATC |
| | | TACAACGCCATCAAGCTGAGCGAGAAGGAGGGCAACATCTTCGACATCCG |
| | | GGACCTGTACAAGCGGACCCTGAAGAACATCGACCTGCTGACCTACAGCT |
| | | TCCCCTGCCAGGACCTGAGCCAGCAGGGCATCCAGAAGGGCATGAAGCG |
| | | GGGCAGCGGCACCCGGAGCGGCCTGCTGTGGGAGATCGAGCGGGCCCTG |
| | | GACAGCACCGAGAAGAACGACCTGCCCAAGTACCTGCTGATGGAGAACGT |
| | | GGGCGCCCTGCTGCACAAGAAGAACGAGGAGGAGCTGAACCAGTGGAAG |
| | | CAGAAGCTGGAGAGCCTGGGCTACCAGAACAGCATCGAGGTGCTGAACG |
| | | CCGCCGACTTCGGCAGCAGCCAGGCCCGGCGGCGGGTGTTCATGATCAGC |
| | | ACCCTGAACGAGTTCGTGGAGCTGCCCAAGGGCGACAAGAAGCCCAAGA |
| | | GCATCAAGAAGGTGCTGAACAAGATCGTGAGCGAGAAGGACATCCTGAA |
| | | CAACCTGCTGAAGTACAACCTGACCGAGTTCAAGAAAACCAAGAGCAACA |
| | | TCAACAAGGCCAGCCTGATCGGCTACAGCAAGTTCAACAGCGAGGGCTAC |
| | | GTGTACGACCCCGAGTTCACCGGCCCCACCCTGACCGCCAGCGGCGCCAA |
| | | CAGCCGGATCAAGATCAAGGACGGCAGCAACATCCGGAAGATGAACAGC |
| | | GACGAGACCTTCCTGTACATCGGCTTCGACAGCCAGGACGGCAAGCGGGT |
| | | GAACGAGATCGAGTTCCTGACCGAGAACCAGAAGATCTTCGTGTGCGGCA |
| | | ACAGCATCAGCGTGGAGGTGCTGGAGGCCATCATCGACAAGATCGGCGGC |
| | | CCCAGCAGCGGCGGCAAGCGGCCCGCCGCCACCAAGAAGGCCGGCCAGG |
| | | CCAAGAAGAAGAAGGGCAGCTACCCCTACGACGTGCCCGACTACGCCTGA |
| | | GCGGCCGCTTAATTAAGCTGCCTTCTGCGGGGCTTGCCTTCTGGCCATGCC |
| | | CTTCTTCTCTCCCTTGCACCTGTACCTCTTGGTCTTTGAATAAAGCCTGAGT |
| | | AGGAAGTCTAGAAAAAAAAAAAAAAAAAAAAAAAAAAAAAAAAAAAAA |
| | | AAAAAAAAAAAAAAAAAAAAAAAAAAAAAAAAAAAAAAAAAAA |
| ZF9- | 63 | AGGAAATAAGAGAGAAAAGAAGAGTAAGAAGAAATATAAGAGCCACCAT |
| MQ1 | | GGCCCCCAAGAAGAAGCGGAAGGTGGGCATCCACGGCGTGCCCGCCGCC |
| (nt) | | GGCAGCAGCGGATCCCTGGAGCCCGGCGAGAAACCTTACAAATGCCCCGA |
| | | GTGCGGCAAGAGCTTCAGCAGAAGCGACGATCTGGTGAGGCACCAAAGA |
| | | ACCCACACCGGCGAAAAACCTTACAAGTGTCCCGAATGCGGAAAGTCCTT |
| | | CAGCAGAGAGGACAATCTGCACACCCACCAGAGAACACACACCGGAGAA |
| | | AAGCCTTACAAGTGCCCCGAATGCGGCAAATCCTTTTCTAGAAGCGATCA |
| | | TCTGACCACCCACCAAAGAACACATACCGGCGAGAAGCCTTACAAATGTC |
| | | CCGAGTGCGGAAAGTCCTTCTCCCAGAGAGCCAATCTGAGGGCTCATCAA |
| | | AGGACCCATACCGGCGAAAAGCCCTACAAATGCCCCGAGTGCGGAAAAT |
| | | CCTTCAGCCAGCTGGCCCATCTGAGAGCCCACCAAAGGACACACACCGGA |
| | | GAGAAACCCTATAAGTGCCCCGAGTGTGGAAAAAGCTTTTCCCAGAGGGC |
| | | CAATCTGAGGGCCCATCAGAGGACCCATACCGGAGAGAAGCCTTATAAAT |
| | | GTCCCGAGTGCGGAAAAAGCTTCAGCGAGAGGAGCCATCTGAGGGAACA |
| | | TCAAAGAACCCACACCGGCGAAAAACCCACCGGAAAAAAGACCAGCGCT |
| | | AGCGGCAGCGGCGGCGGCAGCGGCGGCGCCCGGGACAGCAAGGTGGAGA |
| | | ACAAGACCAAGAAGCTGCGGGTGTTCGAGGCCTTCGCCGGCATCGGCGCC |
| | | CAGCGGAAGGCCCTGGAGAAGGTGCGGAAGGACGAGTACGAGATCGTGG |
| | | GCCTGGCCGAGTGGTACGTGCCCGCCATCGTGATGTACCAGGCCATCCAC |
| | | AACAACTTCCACACCAAGCTGGAGTACAAGAGCGTGAGCCGGGAGGAGA |
| | | TGATCGACTACCTGGAGAACAAGACCCTGAGCTGGAACAGCAAGAACCCC |
| | | GTGAGCAACGGCTACTGGAAGCGGAAGAAGGACGACGAGCTGAAGATCA |
| | | TCTACAACGCCATCAAGCTGAGCGAGAAGGAGGGCAACATCTTCGACATC |
| | | CGGGACCTGTACAAGCGGACCCTGAAGAACATCGACCTGCTGACCTACAG |
| | | CTTCCCCTGCCAGGACCTGAGCCAGCAGGGCATCCAGAAGGGCATGAAGC |
| | | GGGGCAGCGGCACCCGGAGCGGCCTGCTGTGGGAGATCGAGCGGGCCCT |
| | | GGACAGCACCGAGAAGAACGACCTGCCCAAGTACCTGCTGATGGAGAAC |
| | | GTGGGCGCCCTGCTGCACAAGAAGAACGAGGAGGAGCTGAACCAGTGGA |
| | | AGCAGAAGCTGGAGAGCCTGGGCTACCAGAACAGCATCGAGGTGCTGAA |
| | | CGCCGCCGACTTCGGCAGCAGCCAGGCCCGGCGGCGGGTGTTCATGATCA |
| | | GCACCCTGAACGAGTTCGTGGAGCTGCCCAAGGGCGACAAGAAGCCCAA |
| | | GAGCATCAAGAAGGTGCTGAACAAGATCGTGAGCGAGAAGGACATCCTG |
| | | AACAACCTGCTGAAGTACAACCTGACCGAGTTCAAGAAAACCAAGAGCA |
| | | ACATCAACAAGGCCAGCCTGATCGGCTACAGCAAGTTCAACAGCGAGGGC |
| | | TACGTGTACGACCCCGAGTTCACCGGCCCCACCCTGACCGCCAGCGGCGC |
| | | CAACAGCCGGATCAAGATCAAGGACGGCAGCAACATCCGGAAGATGAAC |
| | | AGCGACGAGACCTTCCTGTACATCGGCTTCGACAGCCAGGACGGCAAGCG |
| | | GGTGAACGAGATCGAGTTCCTGACCGAGAACCAGAAGATCTTCGTGTGCG |
| | | GCAACAGCATCAGCGTGGAGGTGCTGGAGGCCATCATCGACAAGATCGGC |
| | | GGCCCCAGCAGCGGCGGCAAGCGGCCCGCCGCCACCAAGAAGGCCGGCC |
| | | AGGCCAAGAAGAAGAAGGGCAGCTACCCCTACGACGTGCCCGACTACGC |
| | | CTGAGCGGCCGCTTAATTAAGCTGCCTTCTGCGGGGCTTGCCTTCTGGCCA |
| | | TGCCCTTCTTCTCTCCCTTGCACCTGTACCTCTTGGTCTTTGAATAAAGCCT |
| | | GAGTAGGAAGTCTAGAAAAAAAAAAAAAAAAAAAAAAAAAAAAAAAAA |
| | | AAAAAAAAAAAAAAAAAAAAAAAAAAAAAAAAAAAAAAAAAAAAAAA |
| ZF10- | 64 | AGTGTGGAAAAAGCTTTAGCCAAAGCGGCGATCTGAGGAGACACCAAAGAACACACACCGGCGAGAAG |
| MQ1 | | CCCTACAAATGTCCCGAGTGCGGAAAGAGCTTCAGCCAGAGCGGCCATCTGACCGAGCATCAGAGAACC |
| (nt) | | CATACCGGCGAAAAACCTTATAAGTGCCCCGAGTGTGGAAAGTCCTTCTCCGAGAGATCCCATCTGAGAG |
| | | AACACCAGAGGACACACACCGGCGAAAAACCTTATAAGTGTCCCGAGTGCGGAAAGTCCTTCAGCGATC |
| | | CCGGCCATCTGGTGAGACATCAAAGGACACATACCGGCGAAAAACCTTATAAGTGTCCCGAATGCGGCA |
| | | AGAGCTTTAGCAGAAACGACACACTCACCGAACACCAGAGGACCCACACCGGCGAGAAACCCTACAAAT |
| | | GCCCCGAGTGCGGCAAATCCTTTTCTAGAGCCGACAATCTGACCGAACACCAGAGGACCCATACCGGAG |
| | | AAAAGCCTTACAAATGTCCCGAGTGTGGCAAATCCTTCTCCACCCATCTGGATCTGATTAGACACCAAAG |
| | | AACACATACCGGAGAAAAGCCCACCGGAAAAAAGACCAGCGCTAGCGGCAGCGGCGGCGGCAGCGGCG |
| | | GCGCCCGGGACAGCAAGGTGGAGAACAAGACCAAGAAGCTGCGGGTGTTCGAGGCCTTCGCCGGCATCG |
| | | GCGCCCAGCGGAAGGCCCTGGAGAAGGTGCGGAAGGACGAGTACGAGATCGTGGGCCTGGCCGAGTGGT |
| | | ACGTGCCCGCCATCGTGATGTACCAGGCCATCCACAACAACTTCCACACCAAGCTGGAGTACAAGAGCGT |
| | | GAGCCGGGAGGAGATGATCGACTACCTGGAGAACAAGACCCTGAGCTGGAACAGCAAGAACCCCGTGAG |
| | | CAACGGCTACTGGAAGCGGAAGAAGGACGACGAGCTGAAGATCATCTACAACGCCATCAAGCTGAGCGA |
| | | GAAGGAGGGCAACATCTTCGACATCCGGGACCTGTACAAGCGGACCCTGAAGAACATCGACCTGCTGAC |
| | | CTACAGCTTCCCCTGCCAGGACCTGAGCCAGCAGGGCATCCAGAAGGGCATGAAGCGGGGCAGCGGCAC |
| | | CCGGAGCGGCCTGCTGTGGGAGATCGAGCGGGCCCTGGACAGCACCGAGAAGAACGACCTGCCCAAGTA |
| | | CCTGCTGATGGAGAACGTGGGCGCCCTGCTGCACAAGAAGAACGAGGAGGAGCTGAACCAGTGGAAGCA |
| | | GAAGCTGGAGAGCCTGGGCTACCAGAACAGCATCGAGGTGCTGAACGCCGCCGACTTCGGCAGCAGCCA |
| | | GGCCCGGCGGCGGGTGTTCATGATCAGCACCCTGAACGAGTTCGTGGAGCTGCCCAAGGGCGACAAGAA |
| | | GCCCAAGAGCATCAAGAAGGTGCTGAACAAGATCGTGAGCGAGAAGGACATCCTGAACAACCTGCTGAA |
| | | GTACAACCTGACCGAGTTCAAGAAAACCAAGAGCAACATCAACAAGGCCAGCCTGATCGGCTACAGCAA |
| | | GTTCAACAGCGAGGGCTACGTGTACGACCCCGAGTTCACCGGCCCCACCCTGACCGCCAGCGGCGCCAAC |
| | | AGCCGGATCAAGATCAAGGACGGCAGCAACATCCGGAAGATGAACAGCGACGAGACCTTCCTGTACATC |
| | | GGCTTCGACAGCCAGGACGGCAAGCGGGTGAACGAGATCGAGTTCCTGACCGAGAACCAGAAGATCTTC |
| | | GTGTGCGGCAACAGCATCAGCGTGGAGGTGCTGGAGGCCATCATCGACAAGATCGGCGGCCCCAGCAGC |
| | | GGCGGCAAGCGGCCCGCCGCCACCAAGAAGGCCGGCCAGGCCAAGAAGAAGAAGGGCAGCTACCCCTA |
| | | CGACGTGCCCGACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAGTCTAGAAAAAA |
| | | AAAAAAAAAAAAAAAAAAAAAAAAAAAAAAAAAAAAAAAAAAAAAAAAAAAAAAAAAAAAAAAAA |
| | | AAAAAAAA |
| ZF11- | 65 | GCCTTATAAGTGCCCCGAGTGTGGCAAATCCTTTTCCGACTGTAGAGATCT |
| MQ1 | | GGCCAGACATCAAAGAACCCACACCGGAGAGAAACCTTATAAATGCCCC |
| (nt) | | GAGTGCGGCAAGTCCTTTAGCCATACCGGCCATCTGCTGGAGCACCAGAG |
| | | GACCCATACCGGCGAGAAGCCTTACAAATGCCCCGAGTGCGGCAAAAGCT |
| | | TCAGCAGAAATGACGCTCTGACCGAGCATCAAAGGACCCATACCGGCGAA |
| | | AAGCCCTACAAGTGTCCCGAGTGTGGAAAGTCCTTCTCCCAGAGCGGCGA |
| | | TCTGAGGAGACACCAGAGAACACACACCGGCGAGAAACCCTATAAATGTC |
| | | CCGAGTGCGGAAAGAGCTTTAGCGACAGCGGCAATCTGAGGGTGCATCAA |
| | | AGAACACACACCGGCGAAAAACCCACCGGAAAAAAGACAAGCGCTAGCG |
| | | GCAGCGGCGGCGGCAGCGGCGGCGCCCGGGACAGCAAGGTGGAGAACAA |
| | | GACCAAGAAGCTGCGGGTGTTCGAGGCCTTCGCCGGCATCGGCGCCCAGC |
| | | GGAAGGCCCTGGAGAAGGTGCGGAAGGACGAGTACGAGATCGTGGGCCT |
| | | GGCCGAGTGGTACGTGCCCGCCATCGTGATGTACCAGGCCATCCACAACA |
| | | ACTTCCACACCAAGCTGGAGTACAAGAGCGTGAGCCGGGAGGAGATGATC |
| | | GACTACCTGGAGAACAAGACCCTGAGCTGGAACAGCAAGAACCCCGTGA |
| | | GCAACGGCTACTGGAAGCGGAAGAAGGACGACGAGCTGAAGATCATCTA |
| | | CAACGCCATCAAGCTGAGCGAGAAGGAGGGCAACATCTTCGACATCCGGG |
| | | ACCTGTACAAGCGGACCCTGAAGAACATCGACCTGCTGACCTACAGCTTC |
| | | CCCTGCCAGGACCTGAGCCAGCAGGGCATCCAGAAGGGCATGAAGCGGG |
| | | GCAGCGGCACCCGGAGCGGCCTGCTGTGGGAGATCGAGCGGGCCCTGGA |
| | | CAGCACCGAGAAGAACGACCTGCCCAAGTACCTGCTGATGGAGAACGTGG |
| | | GCGCCCTGCTGCACAAGAAGAACGAGGAGGAGCTGAACCAGTGGAAGCA |
| | | GAAGCTGGAGAGCCTGGGCTACCAGAACAGCATCGAGGTGCTGAACGCC |
| | | GCCGACTTCGGCAGCAGCCAGGCCCGGCGGCGGGTGTTCATGATCAGCAC |
| | | CCTGAACGAGTTCGTGGAGCTGCCCAAGGGCGACAAGAAGCCCAAGAGC |
| | | ATCAAGAAGGTGCTGAACAAGATCGTGAGCGAGAAGGACATCCTGAACA |
| | | ACCTGCTGAAGTACAACCTGACCGAGTTCAAGAAAACCAAGAGCAACATC |
| | | AACAAGGCCAGCCTGATCGGCTACAGCAAGTTCAACAGCGAGGGCTACGT |
| | | GTACGACCCCGAGTTCACCGGCCCCACCCTGACCGCCAGCGGCGCCAACA |
| | | GCCGGATCAAGATCAAGGACGGCAGCAACATCCGGAAGATGAACAGCGA |
| | | CGAGACCTTCCTGTACATCGGCTTCGACAGCCAGGACGGCAAGCGGGTGA |
| | | ACGAGATCGAGTTCCTGACCGAGAACCAGAAGATCTTCGTGTGCGGCAAC |
| | | AGCATCAGCGTGGAGGTGCTGGAGGCCATCATCGACAAGATCGGCGGCCC |
| | | CAGCAGCGGCGGCAAGCGGCCCGCCGCCACCAAGAAGGCCGGCCAGGCC |
| | | AAGAAGAAGAAGGGCAGCTACCCCTACGACGTGCCCGACTACGCCTGAG |
| | | CGGCCGCTTAATTAAGCTGCCTTCTGCGGGGCTTGCCTTCTGGCCATGCCC |
| | | TTCTTCTCTCCCTTGCACCTGTACCTCTTGGTCTTTGAATAAAGCCTGAGTA |
| | | GGAAGTCTAGAAAAAAAAAAAAAAAAAAAAAAAAAAAAAAAAAAAAAA |
| | | AAAAAAAAAAAAAAAAAAAAAAAAAAAAAAAAAAAAAAAAAA |
| ZF12- | 66 | GACCCATACCGGCGAAAAGCCTTACAAGTGTCCCGAGTGCGGAAAGTCCT |
| MQ1 | | TCTCTAGATCCGACAACCTCGTGAGGCACCAGAGAACCCACACCGGCGAG |
| (nt) | | AAACCTTACAAATGTCCCGAGTGTGGCAAAAGCTTTTCTAGAAGCGACGA |
| | | GCTGGTGAGACATCAAAGAACCCATACCGGCGAAAAACCTTATAAGTGTC |
| | | CCGAGTGCGGCAAATCCTTTAGCCAGCTGGCCCATCTGAGGGCCCACCAG |
| | | AGAACACATACCGGCGAAAAACCCACCGGCAAAAAGACAAGCGCTAGCG |
| | | GCAGCGGCGGCGGCAGCGGCGGCGCCCGGGACAGCAAGGTGGAGAACAA |
| | | GACCAAGAAGCTGCGGGTGTTCGAGGCCTTCGCCGGCATCGGCGCCCAGC |
| | | GGAAGGCCCTGGAGAAGGTGCGGAAGGACGAGTACGAGATCGTGGGCCT |
| | | GGCCGAGTGGTACGTGCCCGCCATCGTGATGTACCAGGCCATCCACAACA |
| | | ACTTCCACACCAAGCTGGAGTACAAGAGCGTGAGCCGGGAGGAGATGATC |
| | | GACTACCTGGAGAACAAGACCCTGAGCTGGAACAGCAAGAACCCCGTGA |
| | | GCAACGGCTACTGGAAGCGGAAGAAGGACGACGAGCTGAAGATCATCTA |
| | | CAACGCCATCAAGCTGAGCGAGAAGGAGGGCAACATCTTCGACATCCGGG |
| | | ACCTGTACAAGCGGACCCTGAAGAACATCGACCTGCTGACCTACAGCTTC |
| | | CCCTGCCAGGACCTGAGCCAGCAGGGCATCCAGAAGGGCATGAAGCGGG |
| | | GCAGCGGCACCCGGAGCGGCCTGCTGTGGGAGATCGAGCGGGCCCTGGA |
| | | CAGCACCGAGAAGAACGACCTGCCCAAGTACCTGCTGATGGAGAACGTGG |
| | | GCGCCCTGCTGCACAAGAAGAACGAGGAGGAGCTGAACCAGTGGAAGCA |
| | | GAAGCTGGAGAGCCTGGGCTACCAGAACAGCATCGAGGTGCTGAACGCC |
| | | GCCGACTTCGGCAGCAGCCAGGCCCGGCGGCGGGTGTTCATGATCAGCAC |
| | | CCTGAACGAGTTCGTGGAGCTGCCCAAGGGCGACAAGAAGCCCAAGAGC |
| | | ATCAAGAAGGTGCTGAACAAGATCGTGAGCGAGAAGGACATCCTGAACA |
| | | ACCTGCTGAAGTACAACCTGACCGAGTTCAAGAAAACCAAGAGCAACATC |
| | | AACAAGGCCAGCCTGATCGGCTACAGCAAGTTCAACAGCGAGGGCTACGT |
| | | GTACGACCCCGAGTTCACCGGCCCCACCCTGACCGCCAGCGGCGCCAACA |
| | | GCCGGATCAAGATCAAGGACGGCAGCAACATCCGGAAGATGAACAGCGA |
| | | CGAGACCTTCCTGTACATCGGCTTCGACAGCCAGGACGGCAAGCGGGTGA |
| | | ACGAGATCGAGTTCCTGACCGAGAACCAGAAGATCTTCGTGTGCGGCAAC |
| | | AGCATCAGCGTGGAGGTGCTGGAGGCCATCATCGACAAGATCGGCGGCCC |
| | | CAGCAGCGGCGGCAAGCGGCCCGCCGCCACCAAGAAGGCCGGCCAGGCC |
| | | AAGAAGAAGAAGGGCAGCTACCCCTACGACGTGCCCGACTACGCCTGAG |
| | | CGGCCGCTTAATTAAGCTGCCTTCTGCGGGGCTTGCCTTCTGGCCATGCCC |
| | | TTCTTCTCTCCCTTGCACCTGTACCTCTTGGTCTTTGAATAAAGCCTGAGTA |
| | | GGAAGTCTAGAAAAAAAAAAAAAAAAAAAAAAAAAAAAAAAAAAAAAA |
| | | AAAAAAAAAAAAAAAAAAAAAAAAAAAAAAAAAAAAAAAAAA |
| ZF10- | 116 | AGGAAATAAGAGAGAAAAGAAGAGTAAGAAGAAATATAAGAGCCACCAT |
| MQ1 | | GGCCCCCAAGAAGAAGCGGAAGGTGGGCATCCACGGCGTGCCCGCCGCC |
| (nt) | | GGCAGCAGCGGATCCCTGGAGCCCGGCGAGAAACCCTACAAGTGCCCCG |
| full | | AGTGTGGAAAAAGCTTTAGCCAAAGCGGCGATCTGAGGAGACACCAAAG |
| length | | AACACACACCGGCGAGAAGCCCTACAAATGTCCCGAGTGCGGAAAGAGC |
| | | TTCAGCCAGAGCGGCCATCTGACCGAGCATCAGAGAACCCATACCGGCGA |
| | | AAAACCTTATAAGTGCCCCGAGTGTGGAAAGTCCTTCTCCGAGAGATCCC |
| | | ATCTGAGAGAACACCAGAGGACACACACCGGCGAAAAACCTTATAAGTGT |
| | | CCCGAGTGCGGAAAGTCCTTCAGCGATCCCGGCCATCTGGTGAGACATCA |
| | | AAGGACACATACCGGCGAAAAACCTTATAAGTGTCCCGAATGCGGCAAGA |
| | | GCTTTAGCAGAAACGACACACTCACCGAACACCAGAGGACCCACACCGGC |
| | | GAGAAACCCTACAAATGCCCCGAGTGCGGCAAATCCTTTTCTAGAGCCGA |
| | | CAATCTGACCGAACACCAGAGGACCCATACCGGAGAAAAGCCTTACAAAT |
| | | GTCCCGAGTGTGGCAAATCCTTCTCCACCCATCTGGATCTGATTAGACACC |
| | | AAAGAACACATACCGGAGAAAAGCCCACCGGAAAAAAGACCAGCGCTAG |
| | | CGGCAGCGGCGGCGGCAGCGGCGGCGCCCGGGACAGCAAGGTGGAGAAC |
| | | AAGACCAAGAAGCTGCGGGTGTTCGAGGCCTTCGCCGGCATCGGCGCCCA |
| | | GCGGAAGGCCCTGGAGAAGGTGCGGAAGGACGAGTACGAGATCGTGGGC |
| | | CTGGCCGAGTGGTACGTGCCCGCCATCGTGATGTACCAGGCCATCCACAA |
| | | CAACTTCCACACCAAGCTGGAGTACAAGAGCGTGAGCCGGGAGGAGATG |
| | | ATCGACTACCTGGAGAACAAGACCCTGAGCTGGAACAGCAAGAACCCCGT |
| | | GAGCAACGGCTACTGGAAGCGGAAGAAGGACGACGAGCTGAAGATCATC |
| | | TACAACGCCATCAAGCTGAGCGAGAAGGAGGGCAACATCTTCGACATCCG |
| | | GGACCTGTACAAGCGGACCCTGAAGAACATCGACCTGCTGACCTACAGCT |
| | | TCCCCTGCCAGGACCTGAGCCAGCAGGGCATCCAGAAGGGCATGAAGCG |
| | | GGGCAGCGGCACCCGGAGCGGCCTGCTGTGGGAGATCGAGCGGGCCCTG |
| | | GACAGCACCGAGAAGAACGACCTGCCCAAGTACCTGCTGATGGAGAACGT |
| | | GGGCGCCCTGCTGCACAAGAAGAACGAGGAGGAGCTGAACCAGTGGAAG |
| | | CAGAAGCTGGAGAGCCTGGGCTACCAGAACAGCATCGAGGTGCTGAACG |
| | | CCGCCGACTTCGGCAGCAGCCAGGCCCGGCGGCGGGTGTTCATGATCAGC |
| | | ACCCTGAACGAGTTCGTGGAGCTGCCCAAGGGCGACAAGAAGCCCAAGA |
| | | GCATCAAGAAGGTGCTGAACAAGATCGTGAGCGAGAAGGACATCCTGAA |
| | | CAACCTGCTGAAGTACAACCTGACCGAGTTCAAGAAAACCAAGAGCAACA |
| | | TCAACAAGGCCAGCCTGATCGGCTACAGCAAGTTCAACAGCGAGGGCTAC |
| | | GTGTACGACCCCGAGTTCACCGGCCCCACCCTGACCGCCAGCGGCGCCAA |
| | | CAGCCGGATCAAGATCAAGGACGGCAGCAACATCCGGAAGATGAACAGC |
| | | GACGAGACCTTCCTGTACATCGGCTTCGACAGCCAGGACGGCAAGCGGGT |
| | | GAACGAGATCGAGTTCCTGACCGAGAACCAGAAGATCTTCGTGTGCGGCA |
| | | ACAGCATCAGCGTGGAGGTGCTGGAGGCCATCATCGACAAGATCGGCGGC |
| | | CCCAGCAGCGGCGGCAAGCGGCCCGCCGCCACCAAGAAGGCCGGCCAGG |
| | | CCAAGAAGAAGAAGGGCAGCTACCCCTACGACGTGCCCGACTACGCCTGA |
| | | GCGGCCGCTTAATTAAGCTGCCTTCTGCGGGGCTTGCCTTCTGGCCATGCC |
| | | CTTCTTCTCTCCCTTGCACCTGTACCTCTTGGTCTTTGAATAAAGCCTGAGT |
| | | AGGAAGTCTAGAAAAAAAAAAAAAAAAAAAAAAAAAAAAAAAAAAAAA |
| | | AAAAAAAAAAAAAAAAAAAAAAAAAAAAAAAAAAAAAAAAAAA |
| ZF11- | 117 | AGGAAATAAGAGAGAAAAGAAGAGTAAGAAGAAATATAAGAGCCACCAT |
| MQ1 | | GGCCCCCAAGAAGAAGCGGAAGGTGGGCATCCACGGCGTGCCCGCCGCC |
| (nt) | | GGCAGCAGCGGATCCCTGGAGCCCGGCGAAAAACCCTATAAGTGCCCCGA |
| full | | ATGTGGAAAGAGCTTCAGCCATACCGGCCATCTGCTGGAACACCAAAGGA |
| length | | CACACACCGGCGAGAAACCTTACAAGTGTCCCGAGTGCGGAAAAAGCTTC |
| | | TCCTCCAAAAAGGCTCTCACCGAGCACCAGAGAACACATACCGGCGAAAA |
| | | GCCTTATAAGTGCCCCGAGTGTGGCAAATCCTTTTCCGACTGTAGAGATCT |
| | | GGCCAGACATCAAAGAACCCACACCGGAGAGAAACCTTATAAATGCCCC |
| | | GAGTGCGGCAAGTCCTTTAGCCATACCGGCCATCTGCTGGAGCACCAGAG |
| | | GACCCATACCGGCGAGAAGCCTTACAAATGCCCCGAGTGCGGCAAAAGCT |
| | | TCAGCAGAAATGACGCTCTGACCGAGCATCAAAGGACCCATACCGGCGAA |
| | | AAGCCCTACAAGTGTCCCGAGTGTGGAAAGTCCTTCTCCCAGAGCGGCGA |
| | | TCTGAGGAGACACCAGAGAACACACACCGGCGAGAAACCCTATAAATGTC |
| | | CCGAGTGCGGAAAGAGCTTTAGCGACAGCGGCAATCTGAGGGTGCATCAA |
| | | AGAACACACACCGGCGAAAAACCCACCGGAAAAAAGACAAGCGCTAGCG |
| | | GCAGCGGCGGCGGCAGCGGCGGCGCCCGGGACAGCAAGGTGGAGAACAA |
| | | GACCAAGAAGCTGCGGGTGTTCGAGGCCTTCGCCGGCATCGGCGCCCAGC |
| | | GGAAGGCCCTGGAGAAGGTGCGGAAGGACGAGTACGAGATCGTGGGCCT |
| | | GGCCGAGTGGTACGTGCCCGCCATCGTGATGTACCAGGCCATCCACAACA |
| | | ACTTCCACACCAAGCTGGAGTACAAGAGCGTGAGCCGGGAGGAGATGATC |
| | | GACTACCTGGAGAACAAGACCCTGAGCTGGAACAGCAAGAACCCCGTGA |
| | | GCAACGGCTACTGGAAGCGGAAGAAGGACGACGAGCTGAAGATCATCTA |
| | | CAACGCCATCAAGCTGAGCGAGAAGGAGGGCAACATCTTCGACATCCGGG |
| | | ACCTGTACAAGCGGACCCTGAAGAACATCGACCTGCTGACCTACAGCTTC |
| | | CCCTGCCAGGACCTGAGCCAGCAGGGCATCCAGAAGGGCATGAAGCGGG |
| | | GCAGCGGCACCCGGAGCGGCCTGCTGTGGGAGATCGAGCGGGCCCTGGA |
| | | CAGCACCGAGAAGAACGACCTGCCCAAGTACCTGCTGATGGAGAACGTGG |
| | | GCGCCCTGCTGCACAAGAAGAACGAGGAGGAGCTGAACCAGTGGAAGCA |
| | | GAAGCTGGAGAGCCTGGGCTACCAGAACAGCATCGAGGTGCTGAACGCC |
| | | GCCGACTTCGGCAGCAGCCAGGCCCGGCGGCGGGTGTTCATGATCAGCAC |
| | | CCTGAACGAGTTCGTGGAGCTGCCCAAGGGCGACAAGAAGCCCAAGAGC |
| | | ATCAAGAAGGTGCTGAACAAGATCGTGAGCGAGAAGGACATCCTGAACA |
| | | ACCTGCTGAAGTACAACCTGACCGAGTTCAAGAAAACCAAGAGCAACATC |
| | | AACAAGGCCAGCCTGATCGGCTACAGCAAGTTCAACAGCGAGGGCTACGT |
| | | GTACGACCCCGAGTTCACCGGCCCCACCCTGACCGCCAGCGGCGCCAACA |
| | | GCCGGATCAAGATCAAGGACGGCAGCAACATCCGGAAGATGAACAGCGA |
| | | CGAGACCTTCCTGTACATCGGCTTCGACAGCCAGGACGGCAAGCGGGTGA |
| | | ACGAGATCGAGTTCCTGACCGAGAACCAGAAGATCTTCGTGTGCGGCAAC |
| | | AGCATCAGCGTGGAGGTGCTGGAGGCCATCATCGACAAGATCGGCGGCCC |
| | | CAGCAGCGGCGGCAAGCGGCCCGCCGCCACCAAGAAGGCCGGCCAGGCC |
| | | AAGAAGAAGAAGGGCAGCTACCCCTACGACGTGCCCGACTACGCCTGAG |
| | | CGGCCGCTTAATTAAGCTGCCTTCTGCGGGGCTTGCCTTCTGGCCATGCCC |
| | | TTCTTCTCTCCCTTGCACCTGTACCTCTTGGTCTTTGAATAAAGCCTGAGTA |
| | | GGAAGTCTAGAAAAAAAAAAAAAAAAAAAAAAAAAAAAAAAAAAAAAA |
| | | AAAAAAAAAAAAAAAAAAAAAAAAAAAAAAAAAAAAAAAAAA |
| ZF12- | 118 | AGGAAATAAGAGAGAAAAGAAGAGTAAGAAGAAATATAAGAGCCACCAT |
| MQ1 | | GGCCCCCAAGAAGAAGCGGAAGGTGGGCATCCACGGCGTGCCCGCCGCC |
| (nt) | | GGCAGCAGCGGATCCCTGGAGCCCGGCGAGAAACCCTATAAATGCCCCGA |
| full | | ATGCGGAAAAAGCTTCAGCCAGTCCAGCTCTCTGGTGAGACATCAGAGGA |
| length | | CACACACCGGCGAAAAGCCTTATAAGTGCCCCGAGTGCGGCAAGTCCTTC |
| | | TCTAGAAGCGATCACCTCACCAATCATCAGAGGACACATACCGGAGAGAA |
| | | GCCCTATAAGTGCCCCGAGTGCGGCAAGAGCTTTAGCCAGCTGGCTCATC |
| | | TGAGAGCTCACCAAAGAACCCATACCGGCGAGAAGCCTTACAAATGCCCC |
| | | GAGTGTGGAAAATCCTTTTCCCAGTCCAGCAACCTCGTCAGACATCAAAG |
| | | GACCCATACCGGCGAAAAGCCTTACAAGTGTCCCGAGTGCGGAAAGTCCT |
| | | TCTCTAGATCCGACAACCTCGTGAGGCACCAGAGAACCCACACCGGCGAG |
| | | AAACCTTACAAATGTCCCGAGTGTGGCAAAAGCTTTTCTAGAAGCGACGA |
| | | GCTGGTGAGACATCAAAGAACCCATACCGGCGAAAAACCTTATAAGTGTC |
| | | CCGAGTGCGGCAAATCCTTTAGCCAGCTGGCCCATCTGAGGGCCCACCAG |
| | | AGAACACATACCGGCGAAAAACCCACCGGCAAAAAGACAAGCGCTAGCG |
| | | GCAGCGGCGGCGGCAGCGGCGGCGCCCGGGACAGCAAGGTGGAGAACAA |
| | | GACCAAGAAGCTGCGGGTGTTCGAGGCCTTCGCCGGCATCGGCGCCCAGC |
| | | GGAAGGCCCTGGAGAAGGTGCGGAAGGACGAGTACGAGATCGTGGGCCT |
| | | GGCCGAGTGGTACGTGCCCGCCATCGTGATGTACCAGGCCATCCACAACA |
| | | ACTTCCACACCAAGCTGGAGTACAAGAGCGTGAGCCGGGAGGAGATGATC |
| | | GACTACCTGGAGAACAAGACCCTGAGCTGGAACAGCAAGAACCCCGTGA |
| | | GCAACGGCTACTGGAAGCGGAAGAAGGACGACGAGCTGAAGATCATCTA |
| | | CAACGCCATCAAGCTGAGCGAGAAGGAGGGCAACATCTTCGACATCCGGG |
| | | ACCTGTACAAGCGGACCCTGAAGAACATCGACCTGCTGACCTACAGCTTC |
| | | CCCTGCCAGGACCTGAGCCAGCAGGGCATCCAGAAGGGCATGAAGCGGG |
| | | GCAGCGGCACCCGGAGCGGCCTGCTGTGGGAGATCGAGCGGGCCCTGGA |
| | | CAGCACCGAGAAGAACGACCTGCCCAAGTACCTGCTGATGGAGAACGTGG |
| | | GCGCCCTGCTGCACAAGAAGAACGAGGAGGAGCTGAACCAGTGGAAGCA |
| | | GAAGCTGGAGAGCCTGGGCTACCAGAACAGCATCGAGGTGCTGAACGCC |
| | | GCCGACTTCGGCAGCAGCCAGGCCCGGCGGCGGGTGTTCATGATCAGCAC |
| | | CCTGAACGAGTTCGTGGAGCTGCCCAAGGGCGACAAGAAGCCCAAGAGC |
| | | ATCAAGAAGGTGCTGAACAAGATCGTGAGCGAGAAGGACATCCTGAACA |
| | | ACCTGCTGAAGTACAACCTGACCGAGTTCAAGAAAACCAAGAGCAACATC |
| | | AACAAGGCCAGCCTGATCGGCTACAGCAAGTTCAACAGCGAGGGCTACGT |
| | | GTACGACCCCGAGTTCACCGGCCCCACCCTGACCGCCAGCGGCGCCAACA |
| | | GCCGGATCAAGATCAAGGACGGCAGCAACATCCGGAAGATGAACAGCGA |
| | | CGAGACCTTCCTGTACATCGGCTTCGACAGCCAGGACGGCAAGCGGGTGA |
| | | ACGAGATCGAGTTCCTGACCGAGAACCAGAAGATCTTCGTGTGCGGCAAC |
| | | AGCATCAGCGTGGAGGTGCTGGAGGCCATCATCGACAAGATCGGCGGCCC |
| | | CAGCAGCGGCGGCAAGCGGCCCGCCGCCACCAAGAAGGCCGGCCAGGCC |
| | | AAGAAGAAGAAGGGCAGCTACCCCTACGACGTGCCCGACTACGCCTGAG |
| | | CGGCCGCTTAATTAAGCTGCCTTCTGCGGGGCTTGCCTTCTGGCCATGCCC |
| | | TTCTTCTCTCCCTTGCACCTGTACCTCTTGGTCTTTGAATAAAGCCTGAGTA |
| | | GGAAGTCTAGAAAAAAAAAAAAAAAAAAAAAAAAAAAAAAAAAAAAAA |
| | | AAAAAAAAAAAAAAAAAAAAAAAAAAAAAAAAAAAAAAAAAA |
| ZF9- | 130 | GGGAAATAAGAGAGAAAAGAAGAGTAAGAAGAAATATAAGAGCCACCAT |
| MQ1 | | GGCCCCCAAGAAGAAGCGGAAGGTGGGCATCCACGGCGTGCCCGCCGCC |
| without | | GGCAGCAGCGGATCCCTGGAGCCCGGCGAGAAACCTTACAAATGCCCCGA |
| HA | | GTGCGGCAAGAGCTTCAGCAGAAGCGACGATCTGGTGAGGCACCAAAGA |
| | | ACCCACACCGGCGAAAAACCTTACAAGTGTCCCGAATGCGGAAAGTCCTT |
| | | CAGCAGAGAGGACAATCTGCACACCCACCAGAGAACACACACCGGAGAA |
| | | AAGCCTTACAAGTGCCCCGAATGCGGCAAATCCTTTTCTAGAAGCGATCA |
| | | TCTGACCACCCACCAAAGAACACATACCGGCGAGAAGCCTTACAAATGTC |
| | | CCGAGTGCGGAAAGTCCTTCTCCCAGAGAGCCAATCTGAGGGCTCATCAA |
| | | AGGACCCATACCGGCGAAAAGCCCTACAAATGCCCCGAGTGCGGAAAAT |
| | | CCTTCAGCCAGCTGGCCCATCTGAGAGCCCACCAAAGGACACACACCGGA |
| | | GAGAAACCCTATAAGTGCCCCGAGTGTGGAAAAAGCTTTTCCCAGAGGGC |
| | | CAATCTGAGGGCCCATCAGAGGACCCATACCGGAGAGAAGCCTTATAAAT |
| | | GTCCCGAGTGCGGAAAAAGCTTCAGCGAGAGGAGCCATCTGAGGGAACA |
| | | TCAAAGAACCCACACCGGCGAAAAACCCACCGGAAAAAAGACCAGCGCT |
| | | AGCGGCAGCGGCGGCGGCAGCGGCGGCGCCCGGGACAGCAAGGTGGAGA |
| | | ACAAGACCAAGAAGCTGCGGGTGTTCGAGGCCTTCGCCGGCATCGGCGCC |
| | | CAGCGGAAGGCCCTGGAGAAGGTGCGGAAGGACGAGTACGAGATCGTGG |
| | | GCCTGGCCGAGTGGTACGTGCCCGCCATCGTGATGTACCAGGCCATCCAC |
| | | AACAACTTCCACACCAAGCTGGAGTACAAGAGCGTGAGCCGGGAGGAGA |
| | | TGATCGACTACCTGGAGAACAAGACCCTGAGCTGGAACAGCAAGAACCCC |
| | | GTGAGCAACGGCTACTGGAAGCGGAAGAAGGACGACGAGCTGAAGATCA |
| | | TCTACAACGCCATCAAGCTGAGCGAGAAGGAGGGCAACATCTTCGACATC |
| | | CGGGACCTGTACAAGCGGACCCTGAAGAACATCGACCTGCTGACCTACAG |
| | | CTTCCCCTGCCAGGACCTGAGCCAGCAGGGCATCCAGAAGGGCATGAAGC |
| | | GGGGCAGCGGCACCCGGAGCGGCCTGCTGTGGGAGATCGAGCGGGCCCT |
| | | GGACAGCACCGAGAAGAACGACCTGCCCAAGTACCTGCTGATGGAGAAC |
| | | GTGGGCGCCCTGCTGCACAAGAAGAACGAGGAGGAGCTGAACCAGTGGA |
| | | AGCAGAAGCTGGAGAGCCTGGGCTACCAGAACAGCATCGAGGTGCTGAA |
| | | CGCCGCCGACTTCGGCAGCAGCCAGGCCCGGCGGCGGGTGTTCATGATCA |
| | | GCACCCTGAACGAGTTCGTGGAGCTGCCCAAGGGCGACAAGAAGCCCAA |
| | | GAGCATCAAGAAGGTGCTGAACAAGATCGTGAGCGAGAAGGACATCCTG |
| | | AACAACCTGCTGAAGTACAACCTGACCGAGTTCAAGAAAACCAAGAGCA |
| | | ACATCAACAAGGCCAGCCTGATCGGCTACAGCAAGTTCAACAGCGAGGGC |
| | | TACGTGTACGACCCCGAGTTCACCGGCCCCACCCTGACCGCCAGCGGCGC |
| | | CAACAGCCGGATCAAGATCAAGGACGGCAGCAACATCCGGAAGATGAAC |
| | | AGCGACGAGACCTTCCTGTACATCGGCTTCGACAGCCAGGACGGCAAGCG |
| | | GGTGAACGAGATCGAGTTCCTGACCGAGAACCAGAAGATCTTCGTGTGCG |
| | | GCAACAGCATCAGCGTGGAGGTGCTGGAGGCCATCATCGACAAGATCGGC |
| | | GGCCCCAGCAGCGGCGGCAAGCGGCCCGCCGCCACCAAGAAGGCCGGCC |
| | | AGGCCAAGAAGAAGAAGGGCAGCTGAGCGGCCGCTTAATTAAGCTGCCTT |
| | | CTGCGGGGCTTGCCTTCTGGCCATGCCCTTCTTCTCTCCCTTGCACCTGTAC |
| | | CTCTTGGTCTTTGAATAAAGCCTGAGTAGGAAGTCTAGAAAAAAAAAAAA |
| | | AAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAA |
| | | AAAAAA |

In some embodiments, an expression repressor comprises a targeting moiety comprising a Zn Finger domain (e.g., comprising an amino acid sequence of any of SEQ ID NO:11-14), and an effector moiety comprising MQ1, e.g., a bacterial MQ1 (e.g., SEQ ID NO: 19). In some embodiments, the expression repressor comprises an amino sequence of any of SEQ ID NOs: 28, 29, 30, 31, 32, 33, 129, and 145-149. The protein sequence of these exemplary expression repressors are disclosed in Table 9. In some embodiments, an expression repressor described herein comprises an amino acid sequence of any of SEQ ID NOs: 28-33, 129 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

TABLE 9
Amino acid sequences of exemplary ZF-MQ1 effectors

| | SEQ | |
|---|---|---|
| NAME | ID NO. | SEQUENCE |
| ZF7- | 28 | MAPKKKRKVGIHGVPAAGSSGSLEPGEKPYKCPECGKSFSRNDALTE |
| MQ1 | | HQRTHTGEKPYKCPECGKSFSDCRDLARHQRTHTGEKPYKCPECGKS |
| (aa) | | FSDPGHLVRHQRTHTGEKPYKCPECGKSFSQSGHLTEHQRTHTGEKP |
| | | YKCPECGKSFSREDNLHTHQRTHTGEKPYKCPECGKSFSTKNSLTEHQ |
| | | RTHTGEKPYKCPECGKSFSRADNLTEHQRTHTGEKPTGKKTSASGSG |
| | | GGSGGARDSKVENKTKKLRVFEAFAGIGAQRKALEKVRKDEYEIVGL |
| | | AEWYVPAIVMYQAIHNNFHTKLEYKSVSREEMIDYLENKTLSWNSKN |
| | | PVSNGYWKRKKDDELKIIYNAIKLSEKEGNIFDIRDLYKRTLKNIDLLT |
| | | YSFPCQDLSQQGIQKGMKRGSGTRSGLLWEIERALDSTEKNDLPKYLL |
| | | MENVGALLHKKNEEELNQWKQKLESLGYQNSIEVLNAADFGSSQAR |
| | | RRVFMISTLNEFVELPKGDKKPKSIKKVLNKIVSEKDILNNLLKYNLTE |
| | | FKKTKSNINKASLIGYSKFNSEGYVYDPEFTGPTLTASGANSRIKIKDG |
| | | SNIRKMNSDETFLYIGFDSQDGKRVNEIEFLTENQKIFVCGNSISVEVL |
| | | EAIIDKIGGPSSGGKRPAATKKAGQAKKKKGSYPYDVPDYA |
| ZF8- | 29 | MAPKKKRKVGIHGVPAAGSSGSLEPGEKPYKCPECGKSFSRSDKLTE |
| MQ1 | | HQRTHTGEKPYKCPECGKSFSRRDELNVHQRTHTGEKPYKCPECGKS |
| (aa) | | FSRSDHLTNHQRTHTGEKPYKCPECGKSFSSPADLTRHQRTHTGEKPY |
| | | KCPECGKSFSRSDHLTNHQRTHTGEKPYKCPECGKSFSSKKALTEHQR |
| | | THTGEKPYKCPECGKSFSTHLDLIRHQRTHTGEKPTGKKTSASGSGGG |
| | | SGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPV |
| | | SNGYWKRKKDDELKIIYNAIKLSEKEGNIFDIRDLYKRTLKNIDLLTYS |
| | | FPCQDLSQQGIQKGMKRGSGTRSGLLWEIERALDSTEKNDLPKYLLM |
| | | ENVGALLHKKNEEELNQWKQKLESLGYQNSIEVLNAADFGSSQARR |
| | | RVFMISTLNEFVELPKGDKKPKSIKKVLNKIVSEKDILNNLLKYNLTEF |
| | | KKTKSNINKASLIGYSKFNSEGYVYDPEFTGPTLTASGANSRIKIKDGS |
| | | NIRKMNSDETFLYIGFDSQDGKRVNEIEFLTENQKIFVCGNSISVEVLE |
| | | AIIDKIGGPSSGGKRPAATKKAGQAKKKKGSYPYDVPDYA |
| ZF9- | 30 | MAPKKKRKVGIHGVPAAGSSGSLEPGEKPYKCPECGKSFSRSDDLVR |
| MQ1 | | HQRTHTGEKPYKCPECGKSFSREDNLHTHQRTHTGEKPYKCPECGKS |
| (aa) | | FSRSDHLTTHQRTHTGEKPYKCPECGKSFSQRANLRAHQRTHTGEKP |
| | | YKCPECGKSFSQLAHLRAHQRTHTGEKPYKCPECGKSFSQRANLRAH |
| | | QRTHTGEKPYKCPECGKSFSERSHLREHQRTHTGEKPTGKKTSASGSG |
| | | GGSGGARDSKVENKTKKLRVFEAFAGIGAQRKALEKVRKDEYEIVGL |
| | | AEWYVPAIVMYQAIHNNFHTKLEYKSVSREEMIDYLENKTLSWNSKN |
| | | PVSNGYWKRKKDDELKIIYNAIKLSEKEGNIFDIRDLYKRTLKNIDLLT |
| | | YSFPCQDLSQQGIQKGMKRGSGTRSGLLWEIERALDSTEKNDLPKYLL |
| | | MENVGALLHKKNEEELNQWKQKLESLGYQNSIEVLNAADFGSSQAR |
| | | RRVFMISTLNEFVELPKGDKKPKSIKKVLNKIVSEKDILNNLLKYNLTE |
| | | FKKTKSNINKASLIGYSKFNSEGYVYDPEFTGPTLTASGANSRIKIKDG |
| | | SNIRKMNSDETFLYIGFDSQDGKRVNEIEFLTENQKIFVCGNSISVEVL |
| | | EAIIDKIGGPSSGGKRPAATKKAGQAKKKKGSYPYDVPDYA |
| ZF10- | 31 | MAPKKKRKVGIHGVPAAGSSGSLEPGEKPYKCPECGKSFSQSGDLRRHQRTHTGEKPYKCPECGKS |
| MQ1 | | FSQSGHLTEHQRTHTGEKPYKCPECGKSFSERSHLREHQRTHTGEKPYKCPECGKSFSDPGHLVRH |
| (aa) | | QRTHTGEKPYKCPECGKSFSRNDTLTEHQRTHTGEKPYKCPECGKSFSRADNLTEHQRTHTGEKPY |
| | | KCPECGKSFSTHLDLIRHQRTHTGEKPTGKKTSASGSGGGSGGARDSKVENKTKKLRVFEAFAGIG |
| | | AQRKALEKVRKDEYEIVGLAEWYVPAIVMYQAIHNNFHTKLEYKSVSREEMIDYLENKTLSWNSK |
| | | NPVSNGYWKRKKDDELKIIYNAIKLSEKEGNIFDIRDLYKRTLKNIDLLTYSFPCQDLSQQGIQKGM |
| | | KRGSGTRSGLLWEIERALDSTEKNDLPKYLLMENVGALLHKKNEEELNQWKQKLESLGYQNSIEV |
| | | LNAADFGSSQARRRVFMISTLNEFVELPKGDKKPKSIKKVLNKIVSEKDILNNLLKYNLTEFKKTKS |
| | | NINKASLIGYSKFNSEGYVYDPEFTGPTLTASGANSRIKIKDGSNIRKMNSDETFLYIGFDSQDGKRV |
| | | NEIEFLTENQKIFVCGNSISVEVLEAIIDKIGGPSSGGKRPAATKKAGQAKKKKGSYPYDVPDYA |
| ZF11- | 32 | MAPKKKRKVGIHGVPAAGSSGSLEPGEKPYKCPECGKSFSHTGHLLE |
| MQ1 | | HQRTHTGEKPYKCPECGKSFSSKKALTEHQRTHTGEKPYKCPECGKS |
| (aa) | | FSDCRDLARHQRTHTGEKPYKCPECGKSFSHTGHLLEHQRTHTGEKP |
| | | YKCPECGKSFSRNDALTEHQRTHTGEKPYKCPECGKSFSQSGDLRRH |
| | | QRTHTGEKPYKCPECGKSFSDSGNLRVHQRTHTGEKPTGKKTSASGS |
| | | GGGSGGARDSKVENKTKKLRVFEAFAGIGAQRKALEKVRKDEYEIVG |
| | | LAEWYVPAIVMYQAIHNNFHTKLEYKSVSREEMIDYLENKTLSWNSK |
| | | NPVSNGYWKRKKDDELKIIYNAIKLSEKEGNIFDIRDLYKRTLKNIDLL |
| | | TYSFPCQDLSQQGIQKGMKRGSGTRSGLLWEIERALDSTEKNDLPKYL |
| | | LMENVGALLHKKNEEELNQWKQKLESLGYQNSIEVLNAADFGSSQA |
| | | RRRVFMISTLNEFVELPKGDKKPKSIKKVLNKIVSEKDILNNLLKYNLT |
| | | EFKKTKSNINKASLIGYSKFNSEGYVYDPEFTGPTLTASGANSRIKIKD |
| | | GSNIRKMNSDETFLYIGFDSQDGKRVNEIEFLTENQKIFVCGNSISVEV |
| | | LEAIIDKIGGPSSGGKRPAATKKAGQAKKKKGSYPYDVPDYA |
| ZF12- | 33 | MAPKKKRKVGIHGVPAAGSSGSLEPGEKPYKCPECGKSFSQSSSLVRH |
| MQ1 | | QRTHTGEKPYKCPECGKSFSRSDHLTNHQRTHTGEKPYKCPECGKSFS |
| (aa) | | QLAHLRAHQRTHTGEKPYKCPECGKSFSQSSNLVRHQRTHTGEKPYK |
| | | CPECGKSFSRSDNLVRHQRTHTGEKPYKCPECGKSFSRSDELVRHQRT |
| | | HTGEKPYKCPECGKSFSQLAHLRAHQRTHTGEKPTGKKTSASGSGGG |
| | | SGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPV |
| | | SNGYWKRKKDDELKIIYNAIKLSEKEGNIFDIRDLYKRTLKNIDLLTYS |
| | | FPCQDLSQQGIQKGMKRGSGTRSGLLWEIERALDSTEKNDLPKYLLM |
| | | ENVGALLHKKNEEELNQWKQKLESLGYQNSIEVLNAADFGSSQARR |
| | | RVFMISTLNEFVELPKGDKKPKSIKKVLNKIVSEKDILNNLLKYNLTEF |
| | | KKTKSNINKASLIGYSKFNSEGYVYDPEFTGPTLTASGANSRIKIKDGS |
| | | NIRKMNSDETFLYIGFDSQDGKRVNEIEFLTENQKIFVCGNSISVEVLE |
| | | AIIDKIGGPSSGGKRPAATKKAGQAKKKKGSYPYDVPDYA |
| ZF9- | 129 | MAPKKKRKVGIHGVPAAGSSGSLEPGEKPYKCPECGKSFSRSDDLVR |
| MQ1 | | HQRTHTGEKPYKCPECGKSFSREDNLHTHQRTHTGEKPYKCPECGKS |
| without | | FSRSDIILTTIIQRTIITGEKPYKCPECGKSFSQRANLRAIIQRTIITGEKP |
| HA | | YKCPECGKSFSQLAHLRAHQRTHTGEKPYKCPECGKSFSQRANLRAH |
| | | QRTHTGEKPYKCPECGKSFSERSHLREHQRTHTGEKPTGKKTSASGSG |
| | | GGSGGARDSKVENKTKKLRVFEAFAGIGAQRKALEKVRKDEYEIVGL |
| | | AEWYVPAIVMYQAIHNNFHTKLEYKSVSREEMIDYLENKTLSWNSKN |
| | | PVSNGYWKRKKDDELKIIYNAIKLSEKEGNIFDIRDLYKRTLKNIDLLT |
| | | YSFPCQDLSQQGIQKGMKRGSGTRSGLLWEIERALDSTEKNDLPKYLL |
| | | MENVGALLHKKNEEELNQWKQKLESLGYQNSIEVLNAADFGSSQAR |
| | | RRVFMISTLNEFVELPKGDKKPKSIKKVLNKIVSEKDILNNLLKYNLTE |
| | | FKKTKSNINKASLIGYSKFNSEGYVYDPEFTGPTLTASGANSRIKIKDG |
| | | SNIRKMNSDETFLYIGFDSQDGKRVNEIEFLTENQKIFVCGNSISVEVL |
| | | EAIIDKIGGPSSGGKRPAATKKAGQAKKKKGS |
| ZF9- | | MAPKKKRKVGIHGVPAAGSSGSLEPGEKPYKCPECGKSFSRSDDLVR |
| MQ1 + | 133 | HQRTHTGEKPYKCPECGKSFSREDNLHTHQRTHTGEKPYKCPECGKS |
| fragment1 | | FSRSDHLTTHQRTHTGEKPYKCPECGKSFSQRANLRAHQRTHTGEKP |
| of | | YKCPECGKSFSQLAHLRAHQRTHTGEKPYKCPECGKSFSQRANLRAH |
| tPT2A | | QRTHTGEKPYKCPECGKSFSERSHLREHQRTHTGEKPTGKKTSASGSG |
| (aa) | | GGSGGARDSKVENKTKKLRVFEAFAGIGAQRKALEKVRKDEYEIVGL |
| | | AEWYVPAIVMYQAIHNNFHTKLEYKSVSREEMIDYLENKTLSWNSKN |
| | | PVSNGYWKRKKDDELKIIYNAIKLSEKEGNIFDIRDLYKRTLKNIDLLT |
| | | YSFPCQDLSQQGIQKGMKRGSGTRSGLLWEIERALDSTEKNDLPKYLL |
| | | MENVGALLHKKNEEELNQWKQKLESLGYQNSIEVLNAADFGSSQAR |
| | | RRVFMISTLNEFVELPKGDKKPKSIKKVLNKIVSEKDILNNLLKYNLTE |
| | | FKKTKSNINKASLIGYSKFNSEGYVYDPEFTGPTLTASGANSRIKIKDG |
| | | SNIRKMNSDETFLYIGFDSQDGKRVNEIEFLTENQKIFVCGNSISVEVL |
| | | EAIIDKIGGPSSGGKRPAATKKAGQAKKKKGSYPYDVPDYAGSYPYD |
| | | VPDYAATNFSLLKQAGDVEENPG |
| ZF7- | 145 | MAPKKKRKVGIHGVPAAGSSGSLEPGEKPYKCPECGKSFSRNDALTE |
| MQ1 | | HQRTHTGEKPYKCPECGKSFSDCRDLARHQRTHTGEKPYKCPECGKS |
| without | | FSDPGHLVRHQRTHTGEKPYKCPECGKSFSQSGHLTEHQRTHTGEKP |
| HA | | YKCPECGKSFSREDNLHTHQRTHTGEKPYKCPECGKSFSTKNSLTEHQ |
| (aa) | | RTHTGEKPYKCPECGKSFSRADNLTEHQRTHTGEKPTGKKTSASGSG |
| | | GGSGGARDSKVENKTKKLRVFEAFAGIGAQRKALEKVRKDEYEIVGL |
| | | AEWYVPAIVMYQAIHNNFHTKLEYKSVSREEMIDYLENKTLSWNSKN |
| | | PVSNGYWKRKKDDELKIIYNAIKLSEKEGNIFDIRDLYKRTLKNIDLLT |
| | | YSFPCQDLSQQGIQKGMKRGSGTRSGLLWEIERALDSTEKNDLPKYLL |
| | | MENVGALLHKKNEEELNQWKQKLESLGYQNSIEVLNAADFGSSQAR |
| | | RRVFMISTLNEFVELPKGDKKPKSIKKVLNKIVSEKDILNNLLKYNLTE |
| | | FKKTKSNINKASLIGYSKFNSEGYVYDPEFTGPTLTASGANSRIKIKDG |
| | | SNIRKMNSDETFLYIGFDSQDGKRVNEIEFLTENQKIFVCGNSISVEVL |
| | | EAIIDKIGGPSSGGKRPAATKKAGQAKKKKGS |
| ZF8- | 146 | MAPKKKRKVGIHGVPAAGSSGSLEPGEKPYKCPECGKSFSRSDKLTE |
| MQ1 | | HQRTHTGEKPYKCPECGKSFSRRDELNVHQRTHTGEKPYKCPECGKS |
| without | | FSRSDHLTNHQRTHTGEKPYKCPECGKSFSSPADLTRHQRTHTGEKPY |
| HA | | KCPECGKSFSRSDHLTNHQRTHTGEKPYKCPECGKSFSSKKALTEHQR |
| (aa) | | THTGEKPYKCPECGKSFSTHLDLIRHQRTHTGEKPTGKKTSASGSGGG |
| | | SGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPV |
| | | SNGYWKRKKDDELKIIYNAIKLSEKEGNIFDIRDLYKRTLKNIDLLTYS |
| | | FPCQDLSQQGIQKGMKRGSGTRSGLLWEIERALDSTEKNDLPKYLLM |
| | | ENVGALLHKKNEEELNQWKQKLESLGYQNSIEVLNAADFGSSQARR |
| | | RVFMISTLNEFVELPKGDKKPKSIKKVLNKIVSEKDILNNLLKYNLTEF |
| | | KKTKSNINKASLIGYSKFNSEGYVYDPEFTGPTLTASGANSRIKIKDGS |
| | | NIRKMNSDETFLYIGFDSQDGKRVNEIEFLTENQKIFVCGNSISVEVLE |
| | | AIIDKIGGPSSGGKRPAATKKAGQAKKKKGS |
| ZF10- | 147 | MAPKKKRKVGIHGVPAAGSSGSLEPGEKPYKCPECGKSFSQSGDLRRHQRTHTGEKPYKCPECGKS |
| MQ1 | | FSQSGHLTEHQRTHTGEKPYKCPECGKSFSERSHLREHQRTHTGEKPYKCPECGKSFSDPGHLVRH |
| without | | QRTHTGEKPYKCPECGKSFSRNDTLTEHQRTHTGEKPYKCPECGKSFSRADNLTEHQRTHTGEKPY |
| HA | | KCPECGKSFSTHLDLIRHQRTHTGEKPTGKKTSASGSGGGSGGARDSKVENKTKKLRVFEAFAGIG |
| (aa) | | AQRKALEKVRKDEYEIVGLAEWYVPAIVMYQAIHNNFHTKLEYKSVSREEMIDYLENKTLSWNSK |
| | | NPVSNGYWKRKKDDELKHIYNAIKLSEKEGNIFDIRDLYKRTLKNIDLLTYSFPCQDLSQQGIQKGM |
| | | KRGSGTRSGLLWEIERALDSTEKNDLPKYLLMENVGALLHKKNEEELNQWKQKLESLGYQNSIEV |
| | | LNAADFGSSQARRRVFMISTLNEFVELPKGDKKPKSIKKVLNKIVSEKDILNNLLKYNLTEFKKTKS |
| | | NINKASLIGYSKFNSEGYVYDPEFTGPTLTASGANSRIKIKDGSNIRKMNSDETFLYIGFDSQDGKRV |
| | | NEIEFLTENQKIFVCGNSISVEVLEAIIDKIGGPSSGGKRPAATKKAGQAKKKKGS |
| ZF11- | 148 | MAPKKKRKVGIHGVPAAGSSGSLEPGEKPYKCPECGKSFSHTGHLLE |
| MQ1 | | HQRTHTGEKPYKCPECGKSFSSKKALTEHQRTHTGEKPYKCPECGKS |
| without | | FSDCRDLARHQRTHTGEKPYKCPECGKSFSHTGHLLEHQRTHTGEKP |
| HA | | YKCPECGKSFSRNDALTEHQRTHTGEKPYKCPECGKSFSQSGDLRRH |
| (aa) | | QRTHTGEKPYKCPECGKSFSDSGNLRVHQRTHTGEKPTGKKTSASGS |
| | | GGGSGGARDSKVENKTKKLRVFEAFAGIGAQRKALEKVRKDEYEIVG |
| | | LAEWYVPAIVMYQAIHNNFHTKLEYKSVSREEMIDYLENKTLSWNSK |
| | | NPVSNGYWKRKKDDELKIIYNAIKLSEKEGNIFDIRDLYKRTLKNIDLL |
| | | TYSFPCQDLSQQGIQKGMKRGSGTRSGLLWEIERALDSTEKNDLPKYL |
| | | LMENVGALLHKKNEEELNQWKQKLESLGYQNSIEVLNAADFGSSQA |
| | | RRRVFMISTLNEFVELPKGDKKPKSIKKVLNKIVSEKDILNNLLKYNLT |
| | | EFKKTKSNINKASLIGYSKFNSEGYVYDPEFTGPTLTASGANSRIKIKD |
| | | GSNIRKMNSDETFLYIGFDSQDGKRVNEIEFLTENQKIFVCGNSISVEV |
| | | LEAIIDKIGGPSSGGKRPAATKKAGQAKKKKGS |
| ZF12- | 149 | MAPKKKRKVGIHGVPAAGSSGSLEPGEKPYKCPECGKSFSQSSSLVRH |
| MQ1 | | QRTHTGEKPYKCPECGKSFSRSDHLTNHQRTHTGEKPYKCPECGKSFS |
| without | | QLAHLRAHQRTHTGEKPYKCPECGKSFSQSSNLVRHQRTHTGEKPYK |
| HA | | CPECGKSFSRSDNLVRHQRTHTGEKPYKCPECGKSFSRSDELVRHQRT |
| (aa) | | HTGEKPYKCPECGKSFSQLAHLRAHQRTHTGEKPTGKKTSASGSGGG |
| | | SGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPV |
| | | SNGYWKRKKDDELKIIYNAIKLSEKEGNIFDIRDLYKRTLKNIDLLTYS |
| | | FPCQDLSQQGIQKGMKRGSGTRSGLLWEIERALDSTEKNDLPKYLLM |
| | | ENVGALLHKKNEEELNQWKQKLESLGYQNSIEVLNAADFGSSQARR |
| | | RVFMISTLNEFVELPKGDKKPKSIKKVLNKIVSEKDILNNLLKYNLTEF |
| | | KKTKSNINKASLIGYSKFNSEGYVYDPEFTGPTLTASGANSRIKIKDGS |
| | | NIRKMNSDETFLYIGFDSQDGKRVNEIEFLTENQKIFVCGNSISVEVLE |
| | | AIIDKIGGPSSGGKRPAATKKAGQAKKKKGS |

In some embodiments an expression repressor comprises a targeting moiety comprising a Zn Finger domain (e.g., having an amino acid sequence of any of SEQ ID NO:11-14), and an effector moiety comprising MQ1, e.g., a bacterial MQ1 (e.g., SEQ ID NO: 87).

In some embodiments, an expression repressor comprises a targeting moiety comprising a Zn Finger domain (e.g., one encoded by a nucleotide sequence of any of SEQ ID NO: 166-168), and an effector moiety comprising MQ1, e.g., a bacterial MQ1 (e.g., one encoded by a nucleotide sequence of SEQ ID NO: 52). In some embodiments, the expression repressors are encoded by the nucleic sequence of SEQ ID NOs: 157, 158, or 159. The nucleic acid sequence of these exemplary expression repressors are disclosed in Table 16. In some embodiments, a nucleic acid described herein comprises a nucleic acid sequence of any of SEQ ID NO: 166-168 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the nucleic acid sequence comprises a poly-A sequence, and in other embodiments, the nucleic acid lacks the poly-A sequence. For example, in some embodiments, a nucleic acid described herein comprises a sequence according to any of SEQ ID NO: 166-168 (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the 3′ poly-A sequence, or comprising a 3′ poly-A sequence of a shorter length.

TABLE 16
Nucleotide sequences of exemplary mouse-specific ZF-MQ1 effectors

| | SEQ | |
|---|---|---|
| | ID | |
| Name | NO. | SEQUENCE |
| ZF15- | 166 | AGGAAATAAGAGAGAAAAGAAGAGTAAGAAGAAATATAAGAGCCACCATGGCCCCCAAGAAGAAGCG |
| MQ1 nt | | GAAGGTGGGCATCCACGGCGTGCCCGCCGCCGGCAGCAGCGGATCCCTTGAGCCCGGAGAAAAGCCAT |
| | | ACAAATGTCCTGAATGCGGAAAGTCATTTTCTACGAGCGGCGAACTCGTGCGGCACCAAAGGACTCATA |
| | | CCGGCGAAAAGCCTTACAAATGCCCGGAGTGCGGAAAAAGCTTCTCCGAGCGCTCGCACTTGCGGGAAC |
| | | ACCAGCGAACCCACACAGGGGAGAAACCGTATAAGTGCCCAGAGTGCGGCAAATCGTTCTCCCGGAAC |
| | | GACACCCTGACCGAACACCAACGCACTCATACTGGCGAAAAACCTTACAAGTGCCCTGAGTGTGGAAAG |
| | | AGCTTCTCCCGCGCCGATAACCTGACCGAGCACCAGCGGACCCATACCGGGGAAAAGCCGTACAAGTGT |
| | | CCGGAATGCGGCAAAAGCTTCAGCACCTCGGGTTCCCTGGTCCGGCATCAGAGAACTCACACCGGAGAG |
| | | AAACCCTATAAGTGTCCTGAGTGCGGGAAGTCCTTTTCATCGCCCGCGGACCTGACTAGACACCAGAGG |
| | | ACCCACACCGGGGAGAAGCCCTACAAGTGCCCCGAATGTGGAAAGTCCTTCTCCGACTCCGGCAACCTC |
| | | CGGGTGCACCAGCGCACCCACACTGGAGAGAAGCCGACCGGAAAGAAAACTTCCGCCTCCGGTTCGGG |
| | | AGGAGGCTCAGGAGGAGCGAGAGATTCCAAGGTCGAGAACAAGACCAAGAAGCTGCGGGTGTTCGAGG |
| | | CCTTTGCTGGCATCGGAGCCCAGAGGAAGGCCCTCGAGAAGGTCCGCAAGGATGAGTACGAGATCGTG |
| | | GGACTCGCGGAGTGGTACGTGCCCGCCATTGTGATGTACCAGGCCATCCATAACAACTTCCACACTAAG |
| | | CTGGAGTACAAGTCCGTGTCCCGGGAGGAAATGATTGACTACCTGGAGAATAAGACCCTGTCATGGAAC |
| | | TCTAAGAACCCCGTGTCGAACGGTTACTGGAAGAGAAAGAAGGATGACGAACTGAAGATTATCTACAA |
| | | CGCGATCAAGCTGAGCGAGAAGGAGGGCAACATCTTCGACATCCGGGACCTCTACAAGCGCACCTTGA |
| | | AGAACATCGATCTGCTGACCTACTCCTTCCCGTGCCAAGACCTGAGCCAGCAGGGCATCCAGAAGGGGA |
| | | TGAAACGGGGCTCCGGTACTCGCAGCGGCTTGCTGTGGGAAATTGAGCGGGCCCTGGATAGCACCGAGA |
| | | AGAACGACTTGCCGAAGTATCTTCTCATGGAAAACGTCGGGGCTCTCCTTCACAAGAAGAACGAGGAAG |
| | | AACTGAACCAGTGGAAGCAAAAGCTGGAATCCCTCGGATACCAGAACTCCATTGAGGTCCTGAACGCCG |
| | | CCGACTTCGGATCGTCGCAAGCCAGACGGAGGGTGTTCATGATTAGCACTCTGAACGAATTCGTGGAAC |
| | | TGCCGAAGGGCGACAAGAAGCCTAAGTCCATCAAGAAGGTGCTGAACAAGATCGTGTCCGAGAAGGAC |
| | | ATTCTCAACAATCTGCTGAAGTACAACCTGACAGAGTTCAAGAAAACCAAGTCCAACATCAACAAGGCC |
| | | TCCTTGATTGGTTACTCAAAGTTCAACAGCGAGGGATACGTGTACGACCCCGAATTCACTGGACCCACTC |
| | | TGACCGCCTCCGGAGCAAACTCTAGGATTAAGATCAAGGACGGCTCCAACATCCGGAAGATGAACTCCG |
| | | ACGAAACCTTTCTGTACATCGGCTTCGACTCGCAAGACGGAAAGCGCGTGAACGAGATCGAATTTCTTA |
| | | CCGAAAACCAGAAGATCTTCGTGTGCGGCAATTCAATCTCCGTGGAAGTCCTGGAAGCGATTATCGACA |
| | | AGATCGGAGGCAGTGGTGGAAAGCGCCCAGCAGCCACTAAGAAGGCCGGACAGGCCAAGAAGAAGAA |
| | | GGGATCCTACCCTTACGATGTGCCGGATTACGCTTGAGCGGCCGCTTAATTAAGCTGCCTTCTGGGGGGC |
| | | TTGCCTTCTGGCCATGCCCTTCTTCTCTCCCTTGCACCTGTACCTCTTGGTCTTTGAATAAAGCCTGAGTA |
| | | GGAAGTCTAGAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAA |
| ZF16- | 167 | AGGAAATAAGAGAGAAAAGAAGAGTAAGAAGAAATATAAGAGCCACCATGGCCCCCAAGAAGAAGCG |
| MQ1 nt | | GAAGGTGGGCATCCACGGCGTGCCCGCCGCCGGCAGCAGCGGATCCCTGGAACCCGGAGAAAAACCTT |
| | | ATAAGTGCCCTGAATGCGGAAAGTCATTCTCGAGGTCGGACAAGCTCGTGCGGCACCAGAGGACACAC |
| | | ACCGGGGAAAAGCCATACAAGTGTCCCGAATGTGGAAAGTCCTTCAGCCAACGCGCCAACCTGAGAGC |
| | | TCATCAGCGGACTCACACTGGCGAAAAACCGTACAAATGCCCCGAATGCGGCAAAAGCTTCTCCCGCGC |
| | | CGACAACTTGACCGAGCACCAGCGGACCCATACCGGCGAAAAGCCGTACAAGTGCCCGGAGTGTGGGA |
| | | AGTCGTTCAGCCAGTCCTCTTCCCTCGTGCGCCACCAACGCACCCATACTGGGGAGAAGCCCTATAAGT |
| | | GTCCTGAGTGTGGCAAATCATTCAGCGATAAGAAGGATCTTACCCGGCACCAACGGACTCATACCGGAG |
| | | AGAAGCCTTACAAGTGCCCCGAGTGCGGAAAGAGCTTCTCGTCCCCGGCGGACCTGACTAGACACCAGC |
| | | GCACCCACACCGGAGAAAAGCCCTACAAGTGCCCAGAGTGCGGGAAGTCCTTTTCCCAATCCGGTCACC |
| | | TGACTGAGCACCAGAGAACCCACACGGGAGAGAAACCGACCGGAAAGAAAACCTCCGCCTCCGGTTCG |
| | | GGAGGAGGCTCAGGAGGAGCGAGAGATTCCAAGGTCGAGAACAAGACCAAGAAGCTGCGGGTGTTCGA |
| | | GGCCTTTGCTGGCATCGGAGCCCAGAGGAAGGCCCTCGAGAAGGTCCGCAAGGATGAGTACGAGATCG |
| | | TGGGACTCGCGGAGTGGTACGTGCCCGCCATTGTGATGTACCAGGCCATCCATAACAACTTCCACACTA |
| | | AGCTGGAGTACAAGTCCGTGTCCCGGGAGGAAATGATTGACTACCTGGAGAATAAGACCCTGTCATGGA |
| | | ACTCTAAGAACCCCGTGTCGAACGGTTACTGGAAGAGAAAGAAGGATGACGAACTGAAGATTATCTAC |
| | | AACGCGATCAAGCTGAGCGAGAAGGAGGGCAACATCTTCGACATCCGGGACCTCTACAAGCGCACCTT |
| | | GAAGAACATCGATCTGCTGACCTACTCCTTCCCGTGCCAAGACCTGAGCCAGCAGGGCATCCAGAAGGG |
| | | GATGAAACGGGGCTCCGGTACTCGCAGCGGCTTGCTGTGGGAAATTGAGCGGGCCCTGGATAGCACCGA |
| | | GAAGAACGACTTGCCGAAGTATCTTCTCATGGAAAACGTCGGGGCTCTCCTTCACAAGAAGAACGAGGA |
| | | AGAACTGAACCAGTGGAAGCAAAAGCTGGAATCCCTCGGATACCAGAACTCCATTGAGGTCCTGAACG |
| | | CCGCCGACTTCGGATCGTCGCAAGCCAGACGGAGGGTGTTCATGATTAGCACTCTGAACGAATTCGTGG |
| | | AACTGCCGAAGGGCGACAAGAAGCCTAAGTCCATCAAGAAGGTGCTGAACAAGATCGTGTCCGAGAAG |
| | | GACATTCTCAACAATCTGCTGAAGTACAACCTGACAGAGTTCAAGAAAACCAAGTCCAACATCAACAAG |
| | | GCCTCCTTGATTGGTTACTCAAAGTTCAACAGCGAGGGATACGTGTACGACCCCGAATTCACTGGACCC |
| | | ACTCTGACCGCCTCCGGAGCAAACTCTAGGATTAAGATCAAGGACGGCTCCAACATCCGGAAGATGAAC |
| | | TCCGACGAAACCTTTCTGTACATCGGCTTCGACTCGCAAGACGGAAAGCGCGTGAACGAGATCGAATTT |
| | | CTTACCGAAAACCAGAAGATCTTCGTGTGCGGCAATTCAATCTCCGTGGAAGTCCTGGAAGCGATTATC |
| | | GACAAGATCGGAGGCAGTGGTGGAAAGCGCCCAGCAGCCACTAAGAAGGCCGGACAGGCCAAGAAGA |
| | | AGAAGGGATCCTACCCTTACGATGTGCCGGATTACGCTTGAGCGGCCGCTTAATTAAGCTGCCTTCTGCG |
| | | GGGCTTGCCTTCTGGCCATGCCCTTCTTCTCTCCCTTGCACCTGTACCTCTTGGTCTTTGAATAAAGCCTG |
| | | AGTAGGAAGTCTAGAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAA |
| ZF17- | 168 | AGGAAATAAGAGAGAAAAGAAGAGTAAGAAGAAATATAAGAGCCACCATGGCCCCCAAGAAGAAGCG |
| MQ1 nt | | GAAGGTGGGCATCCACGGCGTGCCCGCCGCCGGCAGCAGCGGATCCTTGGAACCCGGAGAAAAGCCAT |
| | | ACAAATGCCCCGAATGCGGAAAGTCGTTCAGCCAGTCCGGCGACCTCAGACGGCACCAACGGACTCAC |
| | | ACCGGCGAAAAACCGTACAAGTGCCCAGAGTGCGGCAAAAGCTTTAGCCAGTCGGGCGATCTGCGGAG |
| | | ACATCAGCGCACTCACACTGGTGAAAAGCCCTACAAGTGTCCTGAGTGCGGGAAGTCCTTCAGCGAGCG |
| | | CTCCCATCTTCGCGAGCACCAGAGAACCCACACTGGAGAAAAACCTTATAAGTGCCCTGAGTGTGGCAA |
| | | ATCCTTCTCAACCACCGGCAACCTGACTGTGCACCAGCGGACCCACACAGGGGAGAAGCCTTACAAGTG |
| | | CCCGGAGTGTGGGAAGTCATTCTCCCATCGGACGACCCTGACCAACCACCAGAGGACCCATACTGGCGA |
| | | AAAGCCGTATAAGTGTCCGGAGTGCGGAAAGAGCTTCTCCGACTCCGGAAACCTCAGGGTGCACCAACG |
| | | CACCCACACCGGAGAGAAGCCGTACAAATGTCCCGAATGTGGAAAGTCCTTCTCCCAATCCTCTTCGCT |
| | | GGTCCGGCACCAGCGAACTCATACCGGGGAAAAGCCCACCGGAAAGAAAACCTCGGCCTCCGGTTCGG |
| | | GAGGAGGCTCAGGAGGAGCGAGAGATTCCAAGGTCGAGAACAAGACCAAGAAGCTGCGGGTGTTCGAG |
| | | GCCTTTGCTGGCATCGGAGCCCAGAGGAAGGCCCTCGAGAAGGTCCGCAAGGATGAGTACGAGATCGT |
| | | GGGACTCGCGGAGTGGTACGTGCCCGCCATTGTGATGTACCAGGCCATCCATAACAACTTCCACACTAA |
| | | GCTGGAGTACAAGTCCGTGTCCCGGGAGGAAATGATTGACTACCTGGAGAATAAGACCCTGTCATGGAA |
| | | CTCTAAGAACCCCGTGTCGAACGGTTACTGGAAGAGAAAGAAGGATGACGAACTGAAGATTATCTACA |
| | | ACGCGATCAAGCTGAGCGAGAAGGAGGGCAACATCTTCGACATCCGGGACCTCTACAAGCGCACCTTG |
| | | AAGAACATCGATCTGCTGACCTACTCCTTCCCGTGCCAAGACCTGAGCCAGCAGGGCATCCAGAAGGGG |
| | | ATGAAACGGGGCTCCGGTACTCGCAGCGGCTTGCTGTGGGAAATTGAGCGGGCCCTGGATAGCACCGAG |
| | | AAGAACGACTTGCCGAAGTATCTTCTCATGGAAAACGTCGGGGCTCTCCTTCACAAGAAGAACGAGGAA |
| | | GAACTGAACCAGTGGAAGCAAAAGCTGGAATCCCTCGGATACCAGAACTCCATTGAGGTCCTGAACGCC |
| | | GCCGACTTCGGATCGTCGCAAGCCAGACGGAGGGTGTTCATGATTAGCACTCTGAACGAATTCGTGGAA |
| | | CTGCCGAAGGGCGACAAGAAGCCTAAGTCCATCAAGAAGGTGCTGAACAAGATCGTGTCCGAGAAGGA |
| | | CATTCTCAACAATCTGCTGAAGTACAACCTGACAGAGTTCAAGAAAACCAAGTCCAACATCAACAAGGC |
| | | CTCCTTGATTGGTTACTCAAAGTTCAACAGCGAGGGATACGTGTACGACCCCGAATTCACTGGACCCACT |
| | | CTGACCGCCTCCGGAGCAAACTCTAGGATTAAGATCAAGGACGGCTCCAACATCCGGAAGATGAACTCC |
| | | GACGAAACCTTTCTGTACATCGGCTTCGACTCGCAAGACGGAAAGCGCGTGAACGAGATCGAATTTCTT |
| | | ACCGAAAACCAGAAGATCTTCGTGTGCGGCAATTCAATCTCCGTGGAAGTCCTGGAAGCGATTATCGAC |
| | | AAGATCGGAGGCAGTGGTGGAAAGCGCCCAGCAGCCACTAAGAAGGCCGGACAGGCCAAGAAGAAGA |
| | | AGGGATCCTACCCTTACGATGTGCCGGATTACGCTTGAGCGGCCGCTTAATTAAGCTGCCTTCTGCGGGG |
| | | CTTGCCTTCTGGCCATGCCCTTCTTCTCTCCCTTGCACCTGTACCTCTTGGTCTTTGAATAAAGCCTGAGT |
| | | AGGAAGTCTAGAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAA |

In some embodiments, an expression repressor comprises a targeting moiety comprising a Zn Finger domain (e.g., comprising an amino acid sequence of any of SEQ ID NO:154-156), and an effector moiety comprising MQ1, e.g., a bacterial MQ1 (e.g., SEQ ID NO: 19). In some embodiments, the expression repressor comprises an amino sequence of any of SEQ ID NOs: 160-165. The protein sequences of these exemplary expression repressors are disclosed in Table 17. In some embodiments, an expression repressor described herein comprises an amino acid sequence of any of SEQ ID NOs: 160-165 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

TABLE 17
Amino acid sequences of exemplary ZF-MQ1 effectors

| | SEQ | |
|---|---|---|
| | ID | |
| Name | NO. | SEQUENCE |
| ZF15- | 160 | MAPKKKRKVGIHGVPAAGSSGSLEPGEKPYKCPECGKSFSTSGELVRHQRTHTGEKPYKCPECGKSFSERSHLREH |
| MQ1 | | QRTHTGEKPYKCPECGKSFSRNDTLTEHQRTHTGEKPYKCPECGKSFSRADNLTEHQRTHTGEKPYKCPECGKSFS |
| aa | | TSGSLVRHQRTHTGEKPYKCPECGKSFSSPADLTRHQRTHTGEKPYKCPECGKSFSDSGNLRVHQRTHTGEKPTGK |
| | | KTSASGSGGGSGGARDSKVENKTKKLRVFEAFAGIGAQRKALEKVRKDEYEIVGLAEWYVPAIVMYQAIINNFII |
| | | TKLEYKSVSREEMIDYLENKTLSWNSKNPVSNGYWKRKKDDELKIIYNAIKLSEKEGNIFDIRDLYKRTLKNIDLLT |
| | | YSFPCQDLSQQGIQKGMKRGSGTRSGLLWEIERALDSTEKNDLPKYLLMENVGALLHKKNEEELNQWKQKLESL |
| | | GYQNSIEVLNAADFGSSQARRRVFMISTLNEFVELPKGDKKPKSIKKVLNKIVSEKDILNNLLKYNLTEFKKTKSNI |
| | | NKASLIGYSKFNSEGYVYDPEFTGPTLTASGANSRIKIKDGSNIRKMNSDETFLYIGFDSQDGKRVNEIEFLTENQKI |
| | | FVCGNSISVEVLEAIIDKIGGSGGKRPAATKKAGQAKKKKGSYPYDVPDYA |
| ZF16- | 161 | MAPKKKRKVGIHGVPAAGSSGSLEPGEKPYKCPECGKSFSRSDKLVRHQRTHTGEKPYKCPECGKSFSQRANLRA |
| MQ1 | | HQRTHTGEKPYKCPECGKSFSRADNLTEHQRTHTGEKPYKCPECGKSFSQSSSLVRHQRTHTGEKPYKCPECGKSF |
| aa | | SDKKDLTRHQRTHTGEKPYKCPECGKSFSSPADLTRHQRTHTGEKPYKCPECGKSFSQSGHLTEHQRTHTGEKPTG |
| | | KKTSASGSGGGSGGARDSKVENKTKKLRVFEAFAGIGAQRKALEKVRKDEYEIVGLAEWYVPAIVMYQAIHNNF |
| | | HTKLEYKSVSREEMIDYLENKTLSWNSKNPVSNGYWKRKKDDELKIIYNAIKLSEKEGNIFDIRDLYKRTLKNIDLL |
| | | TYSFPCQDLSQQGIQKGMKRGSGTRSGLLWEIERALDSTEKNDLPKYLLMENVGALLHKKNEEELNQWKQKLES |
| | | LGYQNSIEVLNAADFGSSQARRRVFMISTLNEFVELPKGDKKPKSIKKVLNKIVSEKDILNNLLKYNLTEFKKTKSN |
| | | INKASLIGYSKFNSEGYVYDPEFTGPTLTASGANSRIKIKDGSNIRKMNSDETFLYIGFDSQDGKRVNEIEFLTENQKI |
| | | FVCGNSISVEVLEAIIDKIGGSGGKRPAATKKAGQAKKKKGSYPYDVPDYA |
| ZF17- | 162 | MAPKKKRKVGIHGVPAAGSSGSLEPGEKPYKCPECGKSFSQSGDLRRHQRTHTGEKPYKCPECGKSFSQSGDLRR |
| MQ1 | | HQRTHTGEKPYKCPECGKSFSERSHLREHQRTHTGEKPYKCPECGKSFSTTGNLTVHQRTHTGEKPYKCPECGKSF |
| aa | | SHRTTLTNHQRTHTGEKPYKCPECGKSFSDSGNLRVHQRTHTGEKPYKCPECGKSFSQSSSLVRHQRTHTGEKPTG |
| | | KKTSASGSGGGSGGARDSKVENKTKKLRVFEAFAGIGAQRKALEKVRKDEYEIVGLAEWYVPAIVMYQAIHNNF |
| | | HTKLEYKSVSREEMIDYLENKTLSWNSKNPVSNGYWKRKKDDELKIIYNAIKLSEKEGNIFDIRDLYKRTLKNIDLL |
| | | TYSFPCQDLSQQGIQKGMKRGSGTRSGLLWEIERALDSTEKNDLPKYLLMENVGALLHKKNEEELNQWKQKLES |
| | | LGYQNSIEVLNAADFGSSQARRRVFMISTLNEFVELPKGDKKPKSIKKVLNKIVSEKDILNNLLKYNLTEFKKTKSN |
| | | INKASLIGYSKFNSEGYVYDPEFTGPTLTASGANSRIKIKDGSNIRKMNSDETFLYIGFDSQDGKRVNEIEFLTENQKI |
| | | FVCGNSISVEVLEAIIDKIGGSGGKRPAATKKAGQAKKKKGSYPYDVPDYA |
| ZF15- | 163 | MAPKKKRKVGIHGVPAAGSSGSLEPGEKPYKCPECGKSFSTSGELVRHQRTHTGEKPYKCPECGKSFSERSHLREH |
| MQ1 | | QRTHTGEKPYKCPECGKSFSRNDTLTEHQRTHTGEKPYKCPECGKSFSRADNLTEHQRTHTGEKPYKCPECGKSFS |
| aa | | TSGSLVRHQRTHTGEKPYKCPECGKSFSSPADLTRHQRTHTGEKPYKCPECGKSFSDSGNLRVHQRTHTGEKPTGK |
| without | | KTSASGSGGGSGGARDSKVENKTKKLRVFEAFAGIGAQRKALEKVRKDEYEIVGLAEWYVPAIVMYQAIHNNFH |
| HA tag | | TKLEYKSVSREEMIDYLENKTLSWNSKNPVSNGYWKRKKDDELKITYNAIKLSEKEGNIFDIRDLYKRTLKNIDLLT |
| | | YSFPCQDLSQQGIQKGMKRGSGTRSGLLWEIERALDSTEKNDLPKYLLMENVGALLHKKNEEELNQWKQKLESL |
| | | GYQNSIEVLNAADFGSSQARRRVFMISTLNEFVELPKGDKKPKSIKKVLNKIVSEKDILNNLLKYNLTEFKKTKSNI |
| | | NKASLIGYSKFNSEGYVYDPEFTGPTLTASGANSRIKIKDGSNIRKMNSDETFLYIGFDSQDGKRVNEIEFLTENQKI |
| | | FVCGNSISVEVLEAIIDKIGGSGGKRPAATKKAGQAKKKKGS |
| ZF16- | 164 | MAPKKKRKVGIHGVPAAGSSGSLEPGEKPYKCPECGKSFSRSDKLVRHQRTHTGEKPYKCPECGKSFSQRANLRA |
| MQ1 | | HQRTHTGEKPYKCPECGKSFSRADNLTEHQRTHTGEKPYKCPECGKSFSQSSSLVRHQRTHTGEKPYKCPECGKSF |
| aa | | SDKKDLTRHQRTHTGEKPYKCPECGKSFSSPADLTRHQRTHTGEKPYKCPECGKSFSQSGHLTEHQRTHTGEKPTG |
| without | | KKTSASGSGGGSGGARDSKVENKTKKLRVFEAFAGIGAQRKALEKVRKDEYEIVGLAEWYVPAIVMYQAIHNNF |
| HA tag | | HTKLEYKSVSREEMIDYLENKTLSWNSKNPVSNGYWKRKKDDELKIIYNAIKLSEKEGNIFDIRDLYKRTLKNIDLL |
| | | TYSFPCQDLSQQGIQKGMKRGSGTRSGLLWEIERALDSTEKNDLPKYLLMENVGALLHKKNEEELNQWKQKLES |
| | | LGYQNSIEVLNAADFGSSQARRRVFMISTLNEFVELPKGDKKPKSIKKVLNKIVSEKDILNNLLKYNLTEFKKTKSN |
| | | INKASLIGYSKFNSEGYVYDPEFTGPTLTASGANSRIKIKDGSNIRKMNSDETFLYIGFDSQDGKRVNEIEFLTENQKI |
| | | FVCGNSISVEVLEAIIDKIGGSGGKRPAATKKAGQAKKKKGS |
| ZF17- | 165 | MAPKKKRKVGIHGVPAAGSSGSLEPGEKPYKCPECGKSFSQSGDLRRHQRTHTGEKPYKCPECGKSFSQSGDLRR |
| MQ1 | | HQRTHTGEKPYKCPECGKSFSERSHLREHQRTHTGEKPYKCPECGKSFSTTGNLTVHQRTHTGEKPYKCPECGKSF |
| aa | | SHRTTLTNHQRTHTGEKPYKCPECGKSFSDSGNLRVHQRTHTGEKPYKCPECGKSFSQSSSLVRHQRTHTGEKPTG |
| without | | KKTSASGSGGGSGGARDSKVENKTKKLRVFEAFAGIGAQRKALEKVRKDEYEIVGLAEWYVPAIVMYQAIHNNF |
| HA tag | | HTKLEYKSVSREEMIDYLENKTLSWNSKNPVSNGYWKRKKDDELKIIYNAIKLSEKEGNIFDIRDLYKRTLKNIDLL |
| | | TYSFPCQDLSQQGIQKGMKRGSGTRSGLLWEIERALDSTEKNDLPKYLLMENVGALLHKKNEEELNQWKQKLES |
| | | LGYQNSIEVLNAADFGSSQARRRVFMISTLNEFVELPKGDKKPKSIKKVLNKIVSEKDILNNLLKYNLTEFKKTKSN |
| | | INKASLIGYSKFNSEGYVYDPEFTGPTLTASGANSRIKIKDGSNIRKMNSDETFLYIGFDSQDGKRVNEIEFLTENQKI |
| | | FVCGNSISVEVLEAIIDKIGGSGGKRPAATKKAGQAKKKKGS |

In some embodiments, the present disclosure provides an expression repressor system comprising a first targeting moiety comprising a first ZF, a first effector moiety comprising a DNA methyltransferase, e.g., MQ1 or a functional fragment thereof, a second targeting moiety comprising a second ZF, and a second effector moiety comprising KRAB, e.g., a KRAB domain. In some embodiments, the expression repressor system is encoded by a first nucleic acid encoding the first targeting moiety and first effector moiety, wherein expression is driven by a first promoter or IRES, and a second nucleic acid encoding the second targeting moiety and second effector moiety, wherein expression is driven by a second promoter or IRES. In some embodiments, mono-cistronic sequences are used. In some embodiments, the nucleic acid encoding the expression repressor system is a multi-cistronic sequence. In some embodiments, the multi-cistronic sequence is a bi-cistronic sequence. In some embodiments, the multi-cistronic sequence comprises a sequence encoding the first expression repressor and a sequence encoding the second expression repressor. In some embodiments, the multi-cistronic sequence encodes a self-cleavable peptide sequence, e.g., a 2A peptide sequence, e.g., a T2A peptide sequence, a P2A sequence. In some embodiments, the multi-cistronic sequence encodes a T2A peptide sequence and a P2A peptide sequence. In some embodiments, the multi-cistronic sequence encodes a tandem 2A sequence, e.g., a tPT2A sequence. In some embodiments, the multi-cistronic construct encodes, from 5′ to 3′, (i) a first nuclear localization signal, e.g., a SV40 NLS, (ii) a first targeting moiety, e.g., a DNA binding domain, e.g., a zinc finger binding domain, e.g., ZF-9, (iii) a first effector moiety, e.g., a DNA methyltransferase, e.g., MQ1, (iv) a second nuclear localization signal, e.g., a nucleoplasmin NLS, (v) a linker, e.g., a tPT2A linker, (vi) a third nuclear localization signal, e.g., a SV40NLS, (vii) a second targeting moiety, e.g., a DNA binding domain, e.g., a zinc finger binding domain, e.g., ZF-3, (viii) a second effector moiety, e.g., a transcription repressor moiety, e.g., KRAB, and (ix) a fourth nuclear localization signal, e.g., a nucleoplasmin NLS. In some embodiments, the bi-cistronic construct further comprises a polyA tail. In some embodiments, upon transcription of the bi-cistronic gene construct, a single mRNA transcript encoding the first expression repressor, and the second expression repressor are produced, which upon translation gets cleaved, e.g., after the glycine residue within the 2A peptide, to yield the first expression repressor and the second expression repressor as two separate proteins. In some embodiments, the first and the second expression repressor are separated by “ribosome-skipping”. In some embodiments the first expression repressor and/or the second expression repressor retains a fragment of the 2A peptide after ribosome skipping. In some embodiments, the expression level of the first and second expression repressor are equal. In some embodiments, the expression level of the first and the second expression repressor are different. In some embodiments, the protein level of the first expression repressor is within 1%, 2%, 5%, or 10% of (greater than or less than) the protein level of the second expression repressor.

In some embodiments, a system encoded by a bi-cistronic nucleic acid decreases expression of a target gene (e.g., MYC) at least 1%, at least 2%, at least 3%, at least 4%, at least 5%, at least 10%, at least 20%, at least 30%, at least 40%, at least 50%, in a cell, than an otherwise similar system wherein the first and second expression repressor are encoded by mono-cistronic nucleic acids.

In some embodiments, the bi-cistronic sequence encodes an amino acid of SEQ ID NO: 91, 92, 121, 122, 181, 182, 187, 188, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, an expression repressor system comprises a targeting moiety comprising a Zn Finger domain (e.g., comprising an amino acid sequence of any of SEQ ID NO:7 or 13), and an effector moiety comprising MQ1, e.g., a bacterial MQ1 (e.g., SEQ ID NO: 19) or KRAB, e.g., a KRAB domain (e.g., SEQ ID NO: 18). In some embodiments, the expression repressor comprises an amino sequence of any of SEQ ID NOs: 91, 92, 121, 122, 181, 182, 187, 188. The protein sequence of these exemplary expression repressor systems are disclosed in Table 10. In some embodiments, an expression repressor system described herein comprises an amino acid sequence of any of SEQ ID NOs: 91-92, 121-122, 181, 182, 187, 188, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, the bi-cistronic sequence comprises nucleic acid sequence of SEQ ID NO: 93 or 112 (e.g., a nucleic acid (e.g., cDNA) encoding the expression repressor) or SEQ ID NO: 94 or 113 (e.g., a nucleic acid (e.g., cDNA) encoding the expression repressor). In some embodiments, the bi-cistronic sequence comprises nucleic acid sequence of SEQ ID NO: 196 (e.g., a nucleic acid (e.g., cDNA) encoding the expression repressor) or SEQ ID NO: 197 (e.g., a nucleic acid (e.g., cDNA) encoding the expression repressor). In some embodiments, a nucleic acid described herein comprises a nucleic acid sequence of SEQ ID NO: 93, 94, 112, 113, 196, or 197, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. The nucleic acid sequence encoding these exemplary expression repressor systems are disclosed in Table 10. In some embodiments, the nucleic acid sequence comprises a poly-A sequence, and in other embodiments, the nucleic acid lacks the poly-A sequence.

TABLE 10
Amino acid sequences of, and Nucleic acid sequences encoding, exemplary expression
repressor systems

| | SEQ | |
|---|---|---|
| | ID | |
| Name | NO: | SEQUENCE |
| ZF9- | 91 | MAPKKKRKVGIHGVPAAGSSGSLEPGEKPYKCPECGKSFSRSDDLVRHQRTHTGEKPYKCPECGKSFSREDNLHTHQ |
| MQ1 + ZF | | RTHTGEKPYKCPECGKSFSRSDHLTTHQRTHTGEKPYKCPECGKSFSQRANLRAHQRTHTGEKPYKCPECGKSFSQL |
| 3-KRAB | | AHLRAHQRTHTGEKPYKCPECGKSFSQRANLRAHQRTHTGEKPYKCPECGKSFSERSHLREHQRTHTGEKPTGKKTS |
| (aa) | | ASGSGGGSGGARDSKVENKTKKLRVFEAFAGIGAQRKALEKVRKDEYEIVGLAEWYVPAIVMYQAIHNNFHTKLEY |
| | | KSVSREEMIDYLENKTLSWNSKNPVSNGYWKRKKDDELKIIYNAIKLSEKEGNIFDIRDLYKRTLKNIDLLTYSFPCQD |
| | | LSQQGIQKGMKRGSGTRSGLLWEIERALDSTEKNDLPKYLLMENVGALLHKKNEEELNQWKQKLESLGYQNSIEVL |
| | | NAADFGSSQARRRVFMISTLNEFVELPKGDKKPKSIKKVLNKIVSEKDILNNLLKYNLTEFKKTKSNINKASLIGYSKF |
| | | NSEGYVYDPEFTGPTLTASGANSRIKIKDGSNIRKMNSDETFLYIGFDSQDGKRVNEIEFLTENQKIFVCGNSISVEVLE |
| | | AIIDKIGGPSSGGKRPAATKKAGQAKKKKGSYPYDVPDYAGSYPYDVPDYAATNFSLLKQAGDVEENPGPTSAGKL |
| | | GSGEGRGSLLTCGDVEENPGPLEGSSGSGSPKKKRKVGIHGVPAAGSSGSLEPGEKPYKCPECGKSFSRSDKLVRHQR |
| | | THTGEKPYKCPECGKSFSQRAHLERHQRTHTGEKPYKCPECGKSFSDPGHLVRHQRTHTGEKPYKCPECGKSFSRSD |
| | | KLVRHQRTHTGEKPYKCPECGKSFSQLAHLRAHQRTHTGEKPYKCPECGKSFSRADNLTEHQRTHTGEKPYKCPECG |
| | | KSFSDCRDLARHQRTHTGEKPTGKKTSASGSGGGSGGDAKSLTAWSRTLVTFKDVFVDFTREEWKLLDTAQQILYR |
| | | NVMLENYKNLVSLGYQLTKPDVILRLEKGEEPWLVEREIHQETHPDSETAFEIKSSVSGGKRPAATKKAGQAKKKKG |
| | | SYPYDVPDYA*SSGGKRPAATKKAGQAKKKKGSYPYDVPDYA |
| ZF3- | 92 | MAPKKKRKVGIHGVPAAGSSGSLEPGEKPYKCPECGKSFSRSDKLVRHQRTHTGEKPYKCPECGKSFSQRAHLERHQ |
| KRAB + Z | | RTHTGEKPYKCPECGKSFSDPGHLVRHQRTHTGEKPYKCPECGKSFSRSDKLVRHQRTHTGEKPYKCPECGKSFSQL |
| F9- | | AHLRAHQRTHTGEKPYKCPECGKSFSRADNLTEHQRTHTGEKPYKCPECGKSFSDCRDLARHQRTHTGEKPTGKKTS |
| MQ1(aa) | | ASGSGGGSGGDAKSLTAWSRTLVTFKDVFVDFTREEWKLLDTAQQILYRNVMLENYKNLVSLGYQLTKPDVILRLE |
| | | KGEEPWLVEREIHQETHPDSETAFEIKSSVSSGGKRPAATKKAGQAKKKKGSYPYDVPDYAGSYPYDVPDYAATNFS |
| | | LLKQAGDVEENPGPTSAGKLGSGEGRGSLLTCGDVEENPGPLEGSSGSGSPKKKRKVGIHGVPAAGSSGSLEPGEKPY |
| | | KCPECGKSFSRSDDLVRHQRTHTGEKPYKCPECGKSFSREDNLHTHQRTHTGEKPYKCPECGKSFSRSDHLTTHQRT |
| | | HTGEKPYKCPECGKSFSQRANLRAHQRTHTGEKPYKCPECGKSFSQLAHLRAHQRTHTGEKPYKCPECGKSFSQRAN |
| | | LRAHQRTHTGEKPYKCPECGKSFSERSHLREHQRTHTGEKPTGKKTSASGSGGGSGGARDSKVENKTKKLRVFEAFA |
| | | GIGAQRKALEKVRKDEYEIVGLAEWYVPAIVMYQAIHNNFHTKLEYKSVSREEMIDYLENKTLSWNSKNPVSNGYW |
| | | KRKKDDELKIIYNAIKLSEKEGNIFDIRDLYKRTLKNIDLLTYSFPCQDLSQQGIQKGMKRGSGTRSGLLWEIERALDS |
| | | TEKNDLPKYLLMENVGALLHKKNEEELNQWKQKLESLGYQNSIEVLNAADFGSSQARRRVFMISTLNEFVELPKGD |
| | | KKPKSIKKVLNKIVSEKDILNNLLKYNLTEFKKTKSNINKASLIGYSKFNSEGYVYDPEFTGPTLTASGANSRIKIKDGS |
| | | NIRKMNSDETFLYIGFDSQDGKRVNEIEFLTENQKIFVCGNSISVEVLEAIIDKIGGPSGGKRPAATKKAGQAKKKKGS |
| | | YPYDVPDYA*SSGGKRPAATKKAGQAKKKKGSYPYDVPDYA |
| ZF9- | 93 | GCGGCAAATCCTTTTCTAGAAGCGATCATCTGACCACCCACCAAAGAACACATACCGGCGAGAAGCCTTACAAA |
| MQ1 + ZF | | TGTCCCGAGTGCGGAAAGTCCTTCTCCCAGAGAGCCAATCTGAGGGCTCATCAAAGGACCCATACCGGCGAAAA |
| 3-KRAB | | GCCCTACAAATGCCCCGAGTGCGGAAAATCCTTCAGCCAGCTGGCCCATCTGAGAGCCCACCAAAGGACACACA |
| (nt) | | CCGGAGAGAAACCCTATAAGTGCCCCGAGTGTGGAAAAAGCTTTTCCCAGAGGGCCAATCTGAGGGCCCATCA |
| | | GAGGACCCATACCGGAGAGAAGCCTTATAAATGTCCCGAGTGCGGAAAAAGCTTCAGCGAGAGGAGCCATCTG |
| | | AGGGAACATCAAAGAACCCACACCGGCGAAAAACCCACCGGAAAAAAGACCAGCGCTAGCGGCAGCGGCGGC |
| | | GGCAGCGGCGGCGCCCGGGACAGCAAGGTGGAGAACAAGACCAAGAAGCTGCGGGTGTTCGAGGCCTTCGCCG |
| | | GCATCGGCGCCCAGCGGAAGGCCCTGGAGAAGGTGCGGAAGGACGAGTACGAGATCGTGGGCCTGGCCGAGTG |
| | | GTACGTGCCCGCCATCGTGATGTACCAGGCCATCCACAACAACTTCCACACCAAGCTGGAGTACAAGAGCGTGA |
| | | GCCGGGAGGAGATGATCGACTACCTGGAGAACAAGACCCTGAGCTGGAACAGCAAGAACCCCGTGAGCAACGG |
| | | CTACTGGAAGCGGAAGAAGGACGACGAGCTGAAGATCATCTACAACGCCATCAAGCTGAGCGAGAAGGAGGGC |
| | | AACATCTTCGACATCCGGGACCTGTACAAGCGGACCCTGAAGAACATCGACCTGCTGACCTACAGCTTCCCCTG |
| | | CCAGGACCTGAGCCAGCAGGGCATCCAGAAGGGCATGAAGCGGGGCAGCGGCACCCGGAGCGGCCTGCTGTGG |
| | | GAGATCGAGCGGGCCCTGGACAGCACCGAGAAGAACGACCTGCCCAAGTACCTGCTGATGGAGAACGTGGGCG |
| | | CCCTGCTGCACAAGAAGAACGAGGAGGAGCTGAACCAGTGGAAGCAGAAGCTGGAGAGCCTGGGCTACCAGA |
| | | ACAGCATCGAGGTGCTGAACGCCGCCGACTTCGGCAGCAGCCAGGCCCGGCGGCGGGTGTTCATGATCAGCACC |
| | | CTGAACGAGTTCGTGGAGCTGCCCAAGGGCGACAAGAAGCCCAAGAGCATCAAGAAGGTGCTGAACAAGATCG |
| | | TGAGCGAGAAGGACATCCTGAACAACCTGCTGAAGTACAACCTGACCGAGTTCAAGAAaACCAAGAGCAACAT |
| | | CAACAAGGCCAGCCTGATCGGCTACAGCAAGTTCAACAGCGAGGGCTACGTGTACGACCCCGAGTTCACCGGCC |
| | | CCACCCTGACCGCCAGCGGCGCCAACAGCCGGATCAAGATCAAGGACGGCAGCAACATCCGGAAGATGAACAG |
| | | CGACGAGACCTTCCTGTACATCGGCTTCGACAGCCAGGACGGCAAGCGGGTGAACGAGATCGAGTTCCTGACCG |
| | | AGAACCAGAAGATCTTCGTGTGCGGCAACAGCATCAGCGTGGAGGTGCTGGAGGCCATCATCGACAAGATCGG |
| | | CGGCCCCAGCAGCGGCGGCAAGCGGCCCGCCGCCACCAAGAAGGCCGGCCAGGCCAAGAAGAAGAAGGGCAG |
| | | CTACCCCTACGACGTGCCCGACTACGCCGGGTCCTACCCGTACGACGTGCCCGATTACGCCGCCACCAACTTCTC |
| | | GCTGCTGAAGCAGGCCGGAGATGTGGAAGAAAACCCTGGACCTACCAGTGCCGGAAAGCTCGGTAGCGGAGAG |
| | | GGTCGGGGAAGCCTGCTTACTTGCGGCGACGTGGAAGAGAACCCCGGTCCGCTGGAGGGTTCGTCCGGCTCCGG |
| | | ATCCCCCAAGAAGAAGCGGAAGGTGGGCATCCACGGCGTGCCCGCCGCCGGCAGCAGCGGATCCCTGGAGCCC |
| | | GGCGAAAAACCTTACAAGTGCCCCGAGTGCGGAAAGAGCTTCAGCAGAAGCGACAAACTGGTGAGGCATCAAA |
| | | GGACACATACCGGAGAGAAGCCCTATAAGTGCCCCGAATGTGGCAAATCCTTTTCCCAGAGGGCTCATCTGGAA |
| | | AGACACCAGAGGACCCATACCGGCGAAAAACCCTACAAATGTCCCGAGTGTGGAAAGAGCTTTTCCGATCCCG |
| | | GCCATCTGGTCAGACATCAGAGGACACATACCGGCGAAAAGCCTTACAAGTGTCCCGAATGCGGAAAATCCTTC |
| | | TCCAGAAGCGACAAGCTGGTGAGGCACCAAAGAACCCACACCGGCGAAAAACCCTATAAATGCCCCGAGTGCG |
| | | GCAAGTCCTTTAGCCAGCTGGCCCATCTGAGAGCCCACCAGAGAACACACACCGGAGAGAAGCCTTATAAGTGT |
| | | CCCGAGTGCGGAAAGTCCTTCTCTAGAGCCGACAATCTGACCGAACATCAAAGGACACACACCGGCGAGAAAC |
| | | CTTATAAATGCCCCGAGTGCGGAAAAAGCTTTTCCGACTGCAGAGATCTGGCTAGACACCAGAGAACCCACACC |
| | | GGCGAGAAACCCACCGGCAAAAAGACCAGCGCTAGCGGCAGCGGCGGCGGCAGCGGCGGCGACGCCAAGAGC |
| | | CTGACCGCCTGGAGCCGGACCCTGGTGACCTTCAAGGACGTGTTCGTGGACTTCACCCGGGAGGAGTGGAAGCT |
| | | GCTGGACACCGCCCAGCAGATCCTGTACCGGAACGTGATGCTGGAGAACTACAAGAACCTGGTGAGCCTGGGCT |
| | | ACCAGCTGACCAAGCCCGACGTGATCCTGCGGCTGGAGAAGGGCGAGGAGCCCTGGCTGGTGGAGCGGGAGAT |
| | | CCACCAGGAGACCCACCCCGACAGCGAGACCGCCTTCGAGATCAAGAGCAGCGTGAGCGGCGGCAAGCGGCCC |
| | | GCCGCCACCAAGAAGGCCGGCCAGGCCAAGAAGAAGAAGGGCAGCTACCCCTACGACGTGCCCGACTACGCCT |
| | | GAAGCAGCGGCGGCAAGCGGCCCGCCGCCACCAAGAAGGCCGGCCAGGCCAAGAAGAAGAAGGGCAGCTACC |
| | | CCTACGACGTGCCCGACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAGTCTAGAAAAAAAAAA |
| | | AAAAAAAAAAAAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAA |
| ZF3- | 94 | TATAAATGCCCCGAGTGCGGAAAAAGCTITTCCGACTGCAGAGATCTGGCTAGACACCAGAGAACCCACACCGG |
| KRAB + Z | | CGAGAAACCCACCGGCAAAAAGACCAGCGCTAGCGGCAGCGGCGGCGGCAGCGGCGGCGACGCCAAGAGCCT |
| F9-MQ1 | | GACCGCCTGGAGCCGGACCCTGGTGACCTTCAAGGACGTGTTCGTGGACTTCACCCGGGAGGAGTGGAAGCTGC |
| (nt) | | TGGACACCGCCCAGCAGATCCTGTACCGGAACGTGATGCTGGAGAACTACAAGAACCTGGTGAGCCTGGGCTAC |
| | | CAGCTGACCAAGCCCGACGTGATCCTGCGGCTGGAGAAGGGCGAGGAGCCCTGGCTGGTGGAGCGGGAGATCC |
| | | ACCAGGAGACCCACCCCGACAGCGAGACCGCCTTCGAGATCAAGAGCAGCGTGAGCAGCGGCGGCAAGCGGCC |
| | | CGCCGCCACCAAGAAGGCCGGCCAGGCCAAGAAGAAGAAGGGCAGCTACCCCTACGACGTGCCCGACTACGCC |
| | | GGGTCCTACCCGTACGACGTGCCCGATTACGCCGCCACCAACTTCTCGCTGCTGAAGCAGGCCGGAGATGTGGA |
| | | AGAAAACCCTGGACCTACCAGTGCCGGAAAGCTCGGTAGCGGAGAGGGTCGGGGAAGCCTGCTTACTTGCGGC |
| | | GACGTGGAAGAGAACCCCGGTCCGCTGGAGGGTTCGTCCGGCTCCGGATCCCCCAAGAAGAAGCGGAAGGTGG |
| | | GCATCCACGGCGTGCCCGCCGCCGGCAGCAGCGGATCCCTGGAGCCCGGCGAGAAACCTTACAAATGCCCCGA |
| | | GTGCGGCAAGAGCTTCAGCAGAAGCGACGATCTGGTGAGGCACCAAAGAACCCACACCGGCGAAAAACCTTAC |
| | | AAGTGTCCCGAATGCGGAAAGTCCTTCAGCAGAGAGGACAATCTGCACACCCACCAGAGAACACACACCGGAG |
| | | AAAAGCCTTACAAGTGCCCCGAATGCGGCAAATCCTTTTCTAGAAGCGATCATCTGACCACCCACCAAAGAACA |
| | | CATACCGGCGAGAAGCCTTACAAATGTCCCGAGTGCGGAAAGTCCTTCTCCCAGAGAGCCAATCTGAGGGCTCA |
| | | TCAAAGGACCCATACCGGCGAAAAGCCCTACAAATGCCCCGAGTGCGGAAAATCCTTCAGCCAGCTGGCCCATC |
| | | TGAGAGCCCACCAAAGGACACACACCGGAGAGAAACCCTATAAGTGCCCCGAGTGTGGAAAAAGCTTTTCCCA |
| | | GAGGGCCAATCTGAGGGCCCATCAGAGGACCCATACCGGAGAGAAGCCTTATAAATGTCCCGAGTGCGGAAAA |
| | | AGCTTCAGCGAGAGGAGCCATCTGAGGGAACATCAAAGAACCCACACCGGCGAAAAACCCACCGGAAAAAAG |
| | | ACCAGCGCTAGCGGCAGCGGCGGCGGCAGCGGCGGCGCCCGGGACAGCAAGGTGGAGAACAAGACCAAGAAG |
| | | CTGCGGGTGTTCGAGGCCTTCGCCGGCATCGGCGCCCAGCGGAAGGCCCTGGAGAAGGTGCGGAAGGACGAGT |
| | | ACGAGATCGTGGGCCTGGCCGAGTGGTACGTGCCCGCCATCGTGATGTACCAGGCCATCCACAACAACTTCCAC |
| | | ACCAAGCTGGAGTACAAGAGCGTGAGCCGGGAGGAGATGATCGACTACCTGGAGAACAAGACCCTGAGCTGGA |
| | | ACAGCAAGAACCCCGTGAGCAACGGCTACTGGAAGCGGAAGAAGGACGACGAGCTGAAGATCATCTACAACGC |
| | | CATCAAGCTGAGCGAGAAGGAGGGCAACATCTTCGACATCCGGGACCTGTACAAGCGGACCCTGAAGAACATC |
| | | GACCTGCTGACCTACAGCTTCCCCTGCCAGGACCTGAGCCAGCAGGGCATCCAGAAGGGCATGAAGCGGGGCA |
| | | GCGGCACCCGGAGCGGCCTGCTGTGGGAGATCGAGCGGGCCCTGGACAGCACCGAGAAGAACGACCTGCCCAA |
| | | GTACCTGCTGATGGAGAACGTGGGCGCCCTGCTGCACAAGAAGAACGAGGAGGAGCTGAACCAGTGGAAGCAG |
| | | AAGCTGGAGAGCCTGGGCTACCAGAACAGCATCGAGGTGCTGAACGCCGCCGACTTCGGCAGCAGCCAGGCCC |
| | | GGCGGCGGGTGTTCATGATCAGCACCCTGAACGAGTTCGTGGAGCTGCCCAAGGGCGACAAGAAGCCCAAGAG |
| | | CATCAAGAAGGTGCTGAACAAGATCGTGAGCGAGAAGGACATCCTGAACAACCTGCTGAAGTACAACCTGACC |
| | | GAGTTCAAGAAAACCAAGAGCAACATCAACAAGGCCAGCCTGATCGGCTACAGCAAGTTCAACAGCGAGGGCT |
| | | ACGTGTACGACCCCGAGTTCACCGGCCCCACCCTGACCGCCAGCGGCGCCAACAGCCGGATCAAGATCAAGGA |
| | | CGGCAGCAACATCCGGAAGATGAACAGCGACGAGACCTTCCTGTACATCGGCTTCGACAGCCAGGACGGCAAG |
| | | CGGGTGAACGAGATCGAGTTCCTGACCGAGAACCAGAAGATCTTCGTGTGCGGCAACAGCATCAGCGTGGAGG |
| | | TGCTGGAGGCCATCATCGACAAGATCGGCGGCCCCAGCGGCGGCAAGCGGCCCGCCGCCACCAAGAAGGCCGG |
| | | CCAGGCCAAGAAGAAGAAGGGCAGCTACCCCTACGACGTGCCCGACTACGCCTGAAGCAGCGGCGGCAAGCGG |
| | | CCCGCCGCCACCAAGAAGGCCGGCCAGGCCAAGAAGAAGAAGGGCAGCTACCCCTACGACGTGCCCGACTACG |
| | | CCTGAGCGGCCGCTTAATTAAGCTGCCTTCTGCGGGGCTTGCCTTCTGGCCATGCCCTTCTTCTCTCCCTTGCACC |
| | | TGTACCTCTTGGTCTTTGAATAAAGCCTGAGTAGGAAGTCTAGAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAA |
| | | AAAAAAAAAAAAAA |
| ZF9- | 112 | AGGAAATAAGAGAGAAAAGAAGAGTAAGAAGAAATATAAGAGCCACCATGGCCCCCAAGAAGAAGCGGAAGG |
| MQ1 + ZF | | TGGGCATCCACGGCGTGCCCGCCGCCGGCAGCAGCGGATCCCTGGAGCCCGGCGAGAAACCTTACAAATGCCCC |
| 3-KRAB | | GAGTGCGGCAAGAGCTTCAGCAGAAGCGACGATCTGGTGAGGCACCAAAGAACCCACACCGGCGAAAAACCTT |
| full nt | | ACAAGTGTCCCGAATGCGGAAAGTCCTTCAGCAGAGAGGACAATCTGCACACCCACCAGAGAACACACACCGG |
| sequence | | AGAAAAGCCTTACAAGTGCCCCGAATGCGGCAAATCCTTTTCTAGAAGCGATCATCTGACCACCCACCAAAGAA |
| | | CACATACCGGCGAGAAGCCTTACAAATGTCCCGAGTGCGGAAAGTCCTTCTCCCAGAGAGCCAATCTGAGGGCT |
| | | CATCAAAGGACCCATACCGGCGAAAAGCCCTACAAATGCCCCGAGTGCGGAAAATCCTTCAGCCAGCTGGCCC |
| | | ATCTGAGAGCCCACCAAAGGACACACACCGGAGAGAAACCCTATAAGTGCCCCGAGTGTGGAAAAAGCTTTTC |
| | | CCAGAGGGCCAATCTGAGGGCCCATCAGAGGACCCATACCGGAGAGAAGCCTTATAAATGTCCCGAGTGCGGA |
| | | AAAAGCTTCAGCGAGAGGAGCCATCTGAGGGAACATCAAAGAACCCACACCGGCGAAAAACCCACCGGAAAA |
| | | AAGACCAGCGCTAGCGGCAGCGGCGGCGGCAGCGGCGGCGCCCGGGACAGCAAGGTGGAGAACAAGACCAAG |
| | | AAGCTGCGGGTGTTCGAGGCCTTCGCCGGCATCGGCGCCCAGCGGAAGGCCCTGGAGAAGGTGCGGAAGGACG |
| | | AGTACGAGATCGTGGGCCTGGCCGAGTGGTACGTGCCCGCCATCGTGATGTACCAGGCCATCCACAACAACTTC |
| | | CACACCAAGCTGGAGTACAAGAGCGTGAGCCGGGAGGAGATGATCGACTACCTGGAGAACAAGACCCTGAGCT |
| | | GGAACAGCAAGAACCCCGTGAGCAACGGCTACTGGAAGCGGAAGAAGGACGACGAGCTGAAGATCATCTACA |
| | | ACGCCATCAAGCTGAGCGAGAAGGAGGGCAACATCTTCGACATCCGGGACCTGTACAAGCGGACCCTGAAGAA |
| | | CATCGACCTGCTGACCTACAGCTTCCCCTGCCAGGACCTGAGCCAGCAGGGCATCCAGAAGGGCATGAAGCGGG |
| | | GCAGCGGCACCCGGAGCGGCCTGCTGTGGGAGATCGAGCGGGCCCTGGACAGCACCGAGAAGAACGACCTGCC |
| | | CAAGTACCTGCTGATGGAGAACGTGGGCGCCCTGCTGCACAAGAAGAACGAGGAGGAGCTGAACCAGTGGAAG |
| | | CAGAAGCTGGAGAGCCTGGGCTACCAGAACAGCATCGAGGTGCTGAACGCCGCCGACTTCGGCAGCAGCCAGG |
| | | CCCGGCGGCGGGTGTTCATGATCAGCACCCTGAACGAGTTCGTGGAGCTGCCCAAGGGCGACAAGAAGCCCAA |
| | | GAGCATCAAGAAGGTGCTGAACAAGATCGTGAGCGAGAAGGACATCCTGAACAACCTGCTGAAGTACAACCTG |
| | | ACCGAGTTCAAGAAaACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCCGGATCAAGATCAAG |
| | | GACGGCAGCAACATCCGGAAGATGAACAGCGACGAGACCTTCCTGTACATCGGCTTCGACAGCCAGGACGGCA |
| | | AGCGGGTGAACGAGATCGAGTTCCTGACCGAGAACCAGAAGATCTTCGTGTGCGGCAACAGCATCAGCGTGGA |
| | | GGTGCTGGAGGCCATCATCGACAAGATCGGCGGCCCCAGCAGCGGCGGCAAGCGGCCCGCCGCCACCAAGAAG |
| | | GCCGGCCAGGCCAAGAAGAAGAAGGGCAGCTACCCCTACGACGTGCCCGACTACGCCGGGTCCTACCCGTACG |
| | | ACGTGCCCGATTACGCCGCCACCAACTTCTCGCTGCTGAAGCAGGCCGGAGATGTGGAAGAAAACCCTGGACCT |
| | | ACCAGTGCCGGAAAGCTCGGTAGCGGAGAGGGTCGGGGAAGCCTGCTTACTTGCGGCGACGTGGAAGAGAACC |
| | | CCGGTCCGCTGGAGGGTTCGTCCGGCTCCGGATCCCCCAAGAAGAAGCGGAAGGTGGGCATCCACGGCGTGCCC |
| | | GCCGCCGGCAGCAGCGGATCCCTGGAGCCCGGCGAAAAACCTTACAAGTGCCCCGAGTGCGGAAAGAGCTTCA |
| | | GCAGAAGCGACAAACTGGTGAGGCATCAAAGGACACATACCGGAGAGAAGCCCTATAAGTGCCCCGAATGTGG |
| | | CAAATCCTTTTCCCAGAGGGCTCATCTGGAAAGACACCAGAGGACCCATACCGGCGAAAAACCCTACAAATGTC |
| | | CCGAGTGTGGAAAGAGCTTTTCCGATCCCGGCCATCTGGTCAGACATCAGAGGACACATACCGGCGAAAAGCCT |
| | | TACAAGTGTCCCGAATGCGGAAAATCCTTCTCCAGAAGCGACAAGCTGGTGAGGCACCAAAGAACCCACACCG |
| | | GCGAAAAACCCTATAAATGCCCCGAGTGCGGCAAGTCCTTTAGCCAGCTGGCCCATCTGAGAGCCCACCAGAGA |
| | | ACACACACCGGAGAGAAGCCTTATAAGTGTCCCGAGTGCGGAAAGTCCTTCTCTAGAGCCGACAATCTGACCGA |
| | | ACATCAAAGGACACACACCGGCGAGAAACCTTATAAATGCCCCGAGTGCGGAAAAAGCTTTTCCGACTGCAGA |
| | | GATCTGGCTAGACACCAGAGAACCCACACCGGCGAGAAACCCACCGGCAAAAAGACCAGCGCTAGCGGCAGCG |
| | | GCGGCGGCAGCGGCGGCGACGCCAAGAGCCTGACCGCCTGGAGCCGGACCCTGGTGACCTTCAAGGACGTGTT |
| | | CGTGGACTTCACCCGGGAGGAGTGGAAGCTGCTGGACACCGCCCAGCAGATCCTGTACCGGAACGTGATGCTGG |
| | | AGAACTACAAGAACCTGGTGAGCCTGGGCTACCAGCTGACCAAGCCCGACGTGATCCTGCGGCTGGAGAAGGG |
| | | CGAGGAGCCCTGGCTGGTGGAGCGGGAGATCCACCAGGAGACCCACCCCGACAGCGAGACCGCCTTCGAGATC |
| | | AAGAGCAGCGTGAGCGGCGGCAAGCGGCCCGCCGCCACCAAGAAGGCCGGCCAGGCCAAGAAGAAGAAGGGC |
| | | AGCTACCCCTACGACGTGCCCGACTACGCCTGAAGCAGCGGCGGCAAGCGGCCCGCCGCCACCAAGAAGGCCG |
| | | GCCAGGCCAAGAAGAAGAAGGGCAGCTACCCCTACGACGTGCCCGACTACGCCTGAGCGGCCGCTTAATTAAG |
| | | CTGCCTTCTGCGGGGCTTGCCTTCTGGCCATGCCCTTCTTCTCTCCCTTGCACCTGTACCTCTTGGTCTTTGAATA |
| | | AAGCCTGAGTAGGAAGTCTAGAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAA |
| ZF3- | 113 | AGGAAATAAGAGAGAAAAGAAGAGTAAGAAGAAATATAAGAGCCACCATGGCCCCCAAGAAGAAGCGGAAGG |
| KRAB + Z | | TGGGCATCCACGGCGTGCCCGCCGCCGGCAGCAGCGGATCCCTGGAGCCCGGCGAAAAACCTTACAAGTGCCCC |
| F9-MQ1 | | GAGTGCGGAAAGAGCTTCAGCAGAAGCGACAAACTGGTGAGGCATCAAAGGACACATACCGGAGAGAAGCCCT |
| full nt | | ATAAGTGCCCCGAATGTGGCAAATCCTTTTCCCAGAGGGCTCATCTGGAAAGACACCAGAGGACCCATACCGGC |
| sequence | | GAAAAACCCTACAAATGTCCCGAGTGTGGAAAGAGCTTTTCCGATCCCGGCCATCTGGTCAGACATCAGAGGAC |
| | | ACATACCGGCGAAAAGCCTTACAAGTGTCCCGAATGCGGAAAATCCTTCTCCAGAAGCGACAAGCTGGTGAGG |
| | | CACCAAAGAACCCACACCGGCGAAAAACCCTATAAATGCCCCGAGTGCGGCAAGTCCTTTAGCCAGCTGGCCCA |
| | | TCTGAGAGCCCACCAGAGAACACACACCGGAGAGAAGCCTTATAAGTGTCCCGAGTGCGGAAAGTCCTTCTCTA |
| | | GAGCCGACAATCTGACCGAACATCAAAGGACACACACCGGCGAGAAACCTTATAAATGCCCCGAGTGCGGAAA |
| | | AAGCTTTTCCGACTGCAGAGATCTGGCTAGACACCAGAGAACCCACACCGGCGAGAAACCCACCGGCAAAAAG |
| | | ACCAGCGCTAGCGGCAGCGGCGGCGGCAGCGGCGGCGACGCCAAGAGCCTGACCGCCTGGAGCCGGACCCTGG |
| | | TGACCTTCAAGGACGTGTTCGTGGACTTCACCCGGGAGGAGTGGAAGCTGCTGGACACCGCCCAGCAGATCCTG |
| | | TACCGGAACGTGATGCTGGAGAACTACAAGAACCTGGTGAGCCTGGGCTACCAGCTGACCAAGCCCGACGTGA |
| | | TCCTGCGGCTGGAGAAGGGCGAGGAGCCCTGGCTGGTGGAGCGGGAGATCCACCAGGAGACCCACCCCGACAG |
| | | CGAGACCGCCTTCGAGATCAAGAGCAGCGTGAGCAGCGGCGGCAAGCGGCCCGCCGCCACCAAGAAGGCCGGC |
| | | CAGGCCAAGAAGAAGAAGGGCAGCTACCCCTACGACGTGCCCGACTACGCCGGGTCCTACCCGTACGACGTGC |
| | | CCGATTACGCCGCCACCAACTTCTCGCTGCTGAAGCAGGCCGGAGATGTGGAAGAAAACCCTGGACCTACCAGT |
| | | GCCGGAAAGCTCGGTAGCGGAGAGGGTCGGGGAAGCCTGCTTACTTGCGGCGACGTGGAAGAGAACCCCGGTC |
| | | CGCTGGAGGGTTCGTCCGGCTCCGGATCCCCCAAGAAGAAGCGGAAGGTGGGCATCCACGGCGTGCCCGCCGC |
| | | CGGCAGCAGCGGATCCCTGGAGCCCGGCGAGAAACCTTACAAATGCCCCGAGTGCGGCAAGAGCTTCAGCAGA |
| | | AGCGACGATCTGGTGAGGCACCAAAGAACCCACACCGGCGAAAAACCTTACAAGTGTCCCGAATGCGGAAAGT |
| | | CCTTCAGCAGAGAGGACAATCTGCACACCCACCAGAGAACACACACCGGAGAAAAGCCTTACAAGTGCCCCGA |
| | | ATGCGGCAAATCCTTTTCTAGAAGCGATCATCTGACCACCCACCAAAGAACACATACCGGCGAGAAGCCTTACA |
| | | AATGTCCCGAGTGCGGAAAGTCCTTCTCCCAGAGAGCCAATCTGAGGGCTCATCAAAGGACCCATACCGGCGAA |
| | | AAGCCCTACAAATGCCCCGAGTGCGGAAAATCCTTCAGCCAGCTGGCCCATCTGAGAGCCCACCAAAGGACAC |
| | | ACACCGGAGAGAAACCCTATAAGTGCCCCGAGTGTGGAAAAAGCTTTTCCCAGAGGGCCAATCTGAGGGCCCA |
| | | TCAGAGGACCCATACCGGAGAGAAGCCTTATAAATGTCCCGAGTGCGGAAAAAGCTTCAGCGAGAGGAGCCAT |
| | | CTGAGGGAACATCAAAGAACCCACACCGGCGAAAAACCCACCGGAAAAAAGACCAGCGCTAGCGGCAGCGGC |
| | | GGCGGCAGCGGCGGCGCCCGGGACAGCAAGGTGGAGAACAAGACCAAGAAGCTGCGGGTGTTCGAGGCCTTCG |
| | | CCGGCATCGGCGCCCAGCGGAAGGCCCTGGAGAAGGTGCGGAAGGACGAGTACGAGATCGTGGGCCTGGCCGA |
| | | GTGGTACGTGCCCGCCATCGTGATGTACCAGGCCATCCACAACAACTTCCACACCAAGCTGGAGTACAAGAGCG |
| | | TGAGCCGGGAGGAGATGATCGACTACCTGGAGAACAAGACCCTGAGCTGGAACAGCAAGAACCCCGTGAGCAA |
| | | CGGCTACTGGAAGCGGAAGAAGGACGACGAGCTGAAGATCATCTACAACGCCATCAAGCTGAGCGAGAAGGAG |
| | | GGCAACATCTTCGACATCCGGGACCTGTACAAGCGGACCCTGAAGAACATCGACCTGCTGACCTACAGCTTCCC |
| | | CTGCCAGGACCTGAGCCAGCAGGGCATCCAGAAGGGCATGAAGCGGGGCAGCGGCACCCGGAGCGGCCTGCTG |
| | | TGGGAGATCGAGCGGGCCCTGGACAGCACCGAGAAGAACGACCTGCCCAAGTACCTGCTGATGGAGAACGTGG |
| | | GCGCCCTGCTGCACAAGAAGAACGAGGAGGAGCTGAACCAGTGGAAGCAGAAGCTGGAGAGCCTGGGCTACCA |
| | | GAACAGCATCGAGGTGCTGAACGCCGCCGACTTCGGCAGCAGCCAGGCCCGGCGGCGGGTGTTCATGATCAGC |
| | | ACCCTGAACGAGTTCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGAAAACCAAGAGCAA |
| | | CATCAACAAGGCCAGCCTGATCGGCTACAGCAAGTTCAACAGCGAGGGCTACGTGTACGACCCCGAGTTCACCG |
| | | GCCCCACCCTGACCGCCAGCGGCGCCAACAGCCGGATCAAGATCAAGGACGGCAGCAACATCCGGAAGATGAA |
| | | CAGCGACGAGACCTTCCTGTACATCGGCTTCGACAGCCAGGACGGCAAGCGGGTGAACGAGATCGAGTTCCTGA |
| | | CCGAGAACCAGAAGATCTTCGTGTGCGGCAACAGCATCAGCGTGGAGGTGCTGGAGGCCATCATCGACAAGAT |
| | | CGGCGGCCCCAGCGGCGGCAAGCGGCCCGCCGCCACCAAGAAGGCCGGCCAGGCCAAGAAGAAGAAGGGCAG |
| | | CTACCCCTACGACGTGCCCGACTACGCCTGAAGCAGCGGCGGCAAGCGGCCCGCCGCCACCAAGAAGGCCGGC |
| | | CAGGCCAAGAAGAAGAAGGGCAGCTACCCCTACGACGTGCCCGACTACGCCTGAGCGGCCGCTTAATTAAGCT |
| | | GCCTTCTGCGGGGCTTGCCTTCTGGCCATGCCCTTCTTCTCTCCCTTGCACCTGTACCTCTTGGTCTTTGAATAAA |
| | | GCCTGAGTAGGAAGTCTAGAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAA |
| ZF09- | 196 | GGGAAATAAGAGAGAAAAGAAGAGTAAGAAGAAATATAAGAGCCACCATGGCCCCAAAGAAGAAGAGAAAG |
| MQ1- | | GTCGGAATTCATGGCGTGCCCGCAGCCGGCAGCAGCGGTTCCCTGGAGCCCGGCGAGAAACCTTACAAATGCCC |
| tPT2A- | | CGAGTGCGGCAAGAGCTTCAGCAGAAGCGACGATCTGGTGAGGCACCAAAGAACCCACACCGGCGAAAAACCT |
| ZF54- | | TACAAGTGTCCCGAATGCGGAAAGTCCTTCAGCAGAGAGGACAATCTGCACACCCACCAGAGAACACACACCG |
| KRAB nt | | GAGAAAAGCCTTACAAGTGCCCCGAATGCGGCAAATCCTTTTCTAGAAGCGATCATCTGACCACCCACCAAAGA |
| | | ACACATACCGGCGAGAAGCCTTACAAATGTCCCGAGTGCGGAAAGTCCTTCTCCCAGAGAGCCAATCTGAGGGC |
| | | TCATCAAAGGACCCATACCGGCGAAAAGCCCTACAAATGCCCCGAGTGCGGAAAATCCTTCAGCCAGCTGGCCC |
| | | ATCTGAGAGCCCACCAAAGGACACACACCGGAGAGAAACCCTATAAGTGCCCCGAGTGTGGAAAAAGCTTTTC |
| | | CCAGAGGGCCAATCTGAGGGCCCATCAGAGGACCCATACCGGAGAGAAGCCTTATAAATGTCCCGAGTGCGGA |
| | | AAAAGCTTCAGCGAGAGGAGCCATCTGAGGGAACATCAAAGAACCCACACCGGCGAAAAACCCACCGGAAAA |
| | | AAGACCAGCGCTAGCGGCAGCGGCGGCGGCAGCGGCGGCGCCCGGGACAGCAAGGTGGAGAACAAGACCAAG |
| | | AAGCTGCGGGTGTTCGAGGCCTTCGCCGGCATCGGCGCCCAGCGGAAGGCCCTGGAGAAGGTGCGGAAGGACG |
| | | AGTACGAGATCGTGGGCCTGGCCGAGTGGTACGTGCCCGCCATCGTGATGTACCAGGCCATCCACAACAACTTC |
| | | CACACCAAGCTGGAGTACAAGAGCGTGAGCCGGGAGGAGATGATCGACTACCTGGAGAACAAGACCCTGAGCT |
| | | GGAACAGCAAGAACCCCGTGAGCAACGGCTACTGGAAGCGGAAGAAGGACGACGAGCTGAAGATCATCTACA |
| | | ACGCCATCAAGCTGAGCGAGAAGGAGGGCAACATCTTCGACATCCGGGACCTGTACAAGCGGACCCTGAAGAA |
| | | CATCGACCTGCTGACCTACAGCTTCCCCTGCCAGGACCTGAGCCAGCAGGGCATCCAGAAGGGCATGAAGCGGG |
| | | GCAGCGGCACCCGGAGCGGCCTGCTGTGGGAGATCGAGCGGGCCCTGGACAGCACCGAGAAGAACGACCTGCC |
| | | CAAGTACCTGCTGATGGAGAACGTGGGCGCCCTGCTGCACAAGAAGAACGAGGAGGAGCTGAACCAGTGGAAG |
| | | CAGAAGCTGGAGAGCCTGGGCTACCAGAACAGCATCGAGGTGCTGAACGCCGCCGACTTCGGCAGCAGCCAGG |
| | | CCCGGCGGCGGGTGTTCATGATCAGCACCCTGAACGAGTTCGTGGAGCTGCCCAAGGGCGACAAGAAGCCCAA |
| | | GAGCATCAAGAAGGTGCTGAACAAGATCGTGAGCGAGAAGGACATCCTGAACAACCTGCTGAAGTACAACCTG |
| | | ACCGAGTTCAAGAAAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCCGGATCAAGATCAAG |
| | | GACGGCAGCAACATCCGGAAGATGAACAGCGACGAGACCTTCCTGTACATCGGCTTCGACAGCCAGGACGGCA |
| | | AGCGGGTGAACGAGATCGAGTTCCTGACCGAGAACCAGAAGATCTTCGTGTGCGGCAACAGCATCAGCGTGGA |
| | | GGTGCTGGAGGCCATCATCGACAAGATCGGCGGCCCCAGCGGCGGCAAGCGGCCCGCCGCCACCAAGAAGGCC |
| | | GGCCAGGCCAAGAAGAAGAAGGGCAGCTACCCCTACGACGTGCCCGACTACGCCGGGTCCGCCACCAACTTCT |
| | | CGCTGCTGAAGCAGGCCGGAGACGTGGAAGAAAACCCTGGACCTACCAGTGCCGGAAAGCTCGGTAGCGGAGA |
| | | GGGTCGGGGAAGCCTGCTTACTTGCGGCGACGTGGAAGAGAACCCCGGTCCGCTGGAGGGTTCGTCCGGCTCCG |
| | | GATCCCCCAAGAAGAAGCGGAAGGTGGGCATCCACGGCGTGCCCGCCGCCGGCAGCAGCGGATCCCTGGAGCC |
| | | TGGAGAGAAACCCTACAAATGCCCGGAATGCGGGAAGTCCTTTTCCGAACGCTCGCACCTGAGGGAACACCAG |
| | | AGAACTCACACCGGCGAAAAACCCTATAAGTGCCCAGAATGCGGAAAGAGCTTTTCACGGTCGGACAACCTCGT |
| | | GCGGCACCAACGCACTCATACCGGAGAGAAGCCGTACAAGTGTCCTGAGTGCGGAAAGTCATTCTCCGACTGCC |
| | | GGGATTTGGCCCGCCACCAAAGAACACACACTGGCGAAAAGCCCTACAAGTGCCCGGAGTGTGGAAAGTCCTT |
| | | CAGCACTTCCGGAGAGCTGGTCCGGCACCAGAGGACCCACACCGGGGAGAAGCCTTACAAATGTCCAGAGTGC |
| | | GGTAAAAGCTTCTCCACCACCGGCAACCTCACCGTGCACCAGCGGACCCACACTGGAGAAAAGCCGTATAAATG |
| | | CCCCGAATGCGGCAAGAGCTTCTCGCGATCCGATAAGCTTGTGCGGCATCAGAGAACGCACACTGGGGAAAAG |
| | | CCTTATAAGTGTCCGGAGTGCGGCAAATCCTTCTCCCGCACTGACACCCTGCGGGACCATCAGCGCACCCATAC |
| | | CGGCAAAAAGACCTCTGCTAGCGGCAGCGGCGGCGGCAGCGGCGGCGCCCGGGACGACGCCAAGAGCCTGACC |
| | | GCCTGGAGCCGGACCCTGGTGACCTTCAAGGACGTGTTCGTGGACTTCACCCGGGAGGAGTGGAAGCTGCTGGA |
| | | CACCGCCCAGCAGATCCTGTACCGGAACGTGATGCTGGAGAACTACAAGAACCTGGTGAGCCTGGGCTACCAGC |
| | | TGACCAAGCCCGACGTGATCCTGCGGCTGGAGAAGGGCGAGGAGCCCTGGCTGGTGGAGCGGGAGATCCACCA |
| | | GGAAACCCACCCCGACAGCGAAACCGCCTTCGAGATCAAGAGCAGCGTGCCCAGCAGCGGCGGCAAGCGGCCC |
| | | GCCGCCACCAAGAAGGCCGGCCAGGCCAAGAAGAAGAAGGGCAGCTACCCCTACGACGTGCCCGACTACGCCT |
| | | GAGCGGCCGCTTAATTAAGCTGCCTTCTGCGGGGCTTGCCTTCTGGCCATGCCCTTCTTCTCTCCCTTGCACCTGT |
| | | ACCTCTTGGTCTTTGAATAAAGCCTGAGTAGGAAGTCTAGAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAA |
| | | AAAAAAAAA |
| ZF54- | 197 | GGGAAATAAGAGAGAAAAGAAGAGTAAGAAGAAATATAAGAGCCACCATGGCCCCAAAGAAGAAGAGAAAG |
| KRAB- | | GTCGGAATTCATGGCGTGCCCGCAGCCGGCAGCAGCGGTTCCCTGGAGCCTGGAGAGAAACCCTACAAATGCCC |
| tPT2A- | | GGAATGCGGGAAGTCCTTTTCCGAACGCTCGCACCTGAGGGAACACCAGAGAACTCACACCGGCGAAAAACCC |
| ZF09- | | TATAAGTGCCCAGAATGCGGAAAGAGCTTTTCACGGTCGGACAACCTCGTGCGGCACCAACGCACTCATACCGG |
| MQ1 nt | | AGAGAAGCCGTACAAGTGTCCTGAGTGCGGAAAGTCATTCTCCGACTGCCGGGATTTGGCCCGCCACCAAAGAA |
| | | CACACACTGGCGAAAAGCCCTACAAGTGCCCGGAGTGTGGAAAGTCCTTCAGCACTTCCGGAGAGCTGGTCCGG |
| | | CACCAGAGGACCCACACCGGGGAGAAGCCTTACAAATGTCCAGAGTGCGGTAAAAGCTTCTCCACCACCGGCA |
| | | ACCTCACCGTGCACCAGCGGACCCACACTGGAGAAAAGCCGTATAAATGCCCCGAATGCGGCAAGAGCTTCTCG |
| | | CGATCCGATAAGCTTGTGCGGCATCAGAGAACGCACACTGGGGAAAAGCCTTATAAGTGTCCGGAGTGCGGCA |
| | | AATCCTTCTCCCGCACTGACACCCTGCGGGACCATCAGCGCACCCATACCGGCAAAAAGACCTCTGCTAGCGGC |
| | | AGCGGCGGCGGCAGCGGCGGCGCCCGGGACGACGCCAAGAGCCTGACCGCCTGGAGCCGGACCCTGGTGACCT |
| | | TCAAGGACGTGTTCGTGGACTTCACCCGGGAGGAGTGGAAGCTGCTGGACACCGCCCAGCAGATCCTGTACCGG |
| | | AACGTGATGCTGGAGAACTACAAGAACCTGGTGAGCCTGGGCTACCAGCTGACCAAGCCCGACGTGATCCTGCG |
| | | GCTGGAGAAGGGCGAGGAGCCCTGGCTGGTGGAGCGGGAGATCCACCAGGAAACCCACCCCGACAGCGAAACC |
| | | GCCTTCGAGATCAAGAGCAGCGTGCCCAGCAGCGGCGGCAAGCGGCCCGCCGCCACCAAGAAGGCCGGCCAGG |
| | | CCAAGAAGAAGAAGGGCAGCTACCCCTACGACGTGCCCGACTACGCCGGGTCCGCCACCAACTTCTCGCTGCTG |
| | | AAGCAGGCCGGAGACGTGGAAGAAAACCCTGGACCTACCAGTGCCGGAAAGCTCGGTAGCGGAGAGGGTCGG |
| | | GGAAGCCTGCTTACTTGCGGCGACGTGGAAGAGAACCCCGGTCCGCTGGAGGGTTCGTCCGGCTCCGGATCCCC |
| | | CAAGAAGAAGCGGAAGGTGGGCATCCACGGCGTGCCCGCCGCCGGCAGCAGCGGATCCCTGGAGCCCGGCGAG |
| | | AAACCTTACAAATGCCCCGAGTGCGGCAAGAGCTTCAGCAGAAGCGACGATCTGGTGAGGCACCAAAGAACCC |
| | | ACACCGGCGAAAAACCTTACAAGTGTCCCGAATGCGGAAAGTCCTTCAGCAGAGAGGACAATCTGCACACCCA |
| | | CCAGAGAACACACACCGGAGAAAAGCCTTACAAGTGCCCCGAATGCGGCAAATCCTTTTCTAGAAGCGATCATC |
| | | TGACCACCCACCAAAGAACACATACCGGCGAGAAGCCTTACAAATGTCCCGAGTGCGGAAAGTCCTTCTCCCAG |
| | | AGAGCCAATCTGAGGGCTCATCAAAGGACCCATACCGGCGAAAAGCCCTACAAATGCCCCGAGTGCGGAAAAT |
| | | CCTTCAGCCAGCTGGCCCATCTGAGAGCCCACCAAAGGACACACACCGGAGAGAAACCCTATAAGTGCCCCGA |
| | | GTGTGGAAAAAGCTTTTCCCAGAGGGCCAATCTGAGGGCCCATCAGAGGACCCATACCGGAGAGAAGCCTTAT |
| | | AAATGTCCCGAGTGCGGAAAAAGCTTCAGCGAGAGGAGCCATCTGAGGGAACATCAAAGAACCCACACCGGCG |
| | | AAAAACCCACCGGAAAAAAGACCAGCGCTAGCGGCAGCGGCGGCGGCAGCGGCGGCGCCCGGGACAGCAAGG |
| | | TGGAGAACAAGACCAAGAAGCTGCGGGTGTTCGAGGCCTTCGCCGGCATCGGCGCCCAGCGGAAGGCCCTGGA |
| | | GAAGGTGCGGAAGGACGAGTACGAGATCGTGGGCCTGGCCGAGTGGTACGTGCCCGCCATCGTGATGTACCAG |
| | | GCCATCCACAACAACTTCCACACCAAGCTGGAGTACAAGAGCGTGAGCCGGGAGGAGATGATCGACTACCTGG |
| | | AGAACAAGACCCTGAGCTGGAACAGCAAGAACCCCGTGAGCAACGGCTACTGGAAGCGGAAGAAGGACGACG |
| | | AGCTGAAGATCATCTACAACGCCATCAAGCTGAGCGAGAAGGAGGGCAACATCTTCGACATCCGGGACCTGTA |
| | | CAAGCGGACCCTGAAGAACATCGACCTGCTGACCTACAGCTTCCCCTGCCAGGACCTGAGCCAGCAGGGCATCC |
| | | AGAAGGGCATGAAGCGGGGCAGCGGCACCCGGAGCGGCCTGCTGTGGGAGATCGAGCGGGCCCTGGACAGCAC |
| | | CGAGAAGAACGACCTGCCCAAGTACCTGCTGATGGAGAACGTGGGCGCCCTGCTGCACAAGAAGAACGAGGAG |
| | | GAGCTGAACCAGTGGAAGCAGAAGCTGGAGAGCCTGGGCTACCAGAACAGCATCGAGGTGCTGAACGCCGCCG |
| | | ACTTCGGCAGCAGCCAGGCCCGGCGGCGGGTGTTCATGATCAGCACCCTGAACGAGTTCGTGGAGCTGCCCAAG |
| | | GGCGACAAGAAGCCCAAGAGCATCAAGAAGGTGCTGAACAAGATCGTGAGCGAGAAGGACATCCTGAACAAC |
| | | CTGCTGAAGTACAACCTGACCGAGTTCAAGAAAACCAAGAGCAACATCAACAAGGCCAGCCTGATCGGCTACA |
| | | GCAAGTTCAACAGCGAGGGCTACGTGTACGACCCCGAGTTCACCGGCCCCACCCTGACCGCCAGCGGCGCCAAC |
| | | AGCCGGATCAAGATCAAGGACGGCAGCAACATCCGGAAGATGAACAGCGACGAGACCTTCCTGTACATCGGCT |
| | | TCGACAGCCAGGACGGCAAGCGGGTGAAGGAGATCGAGTTCCTGACCGAGAACCAGAAGATCTTCGTGTGCGG |
| | | CAACAGCATCAGCGTGGAGGTGCTGGAGGCCATCATCGACAAGATCGGCGGCCCCAGCGGCGGCAAGCGGCCC |
| | | GCCGCCACCAAGAAGGCCGGCCAGGCCAAGAAGAAGAAGGGCAGCTACCCCTACGACGTGCCCGACTACGCCT |
| | | GAGCGGCCGCTTAATTAAGCTGCCTTCTGCGGGGCTTGCCTTCTGGCCATGCCCTTCTTCTCTCCCTTGCACCTGT |
| | | ACCTCTTGGTCTTTGAATAAAGCCTGAGTAGGAAGTCTAGAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAA |
| | | AAAAAAAAA |
| ZF9- | 121 | MAPKKKRKVGIHGVPAAGSSGSLEPGEKPYKCPECGKSFSRSDDLVRHQRTHTGEKPYKCPECGKSFSREDNLHTHQ |
| MQ1 + ZF | | RTHTGEKPYKCPECGKSFSRSDHLTTHQRTHTGEKPYKCPECGKSFSQRANLRAHQRTHTGEKPYKCPECGKSFSQL |
| 3-KRAB | | AHLRAHQRTHTGEKPYKCPECGKSFSQRANLRAHQRTHTGEKPYKCPECGKSFSERSHLREHQRTHTGEKPTGKKTS |
| (aa) | | ASGSGGGSGGARDSKVENKTKKLRVFEAFAGIGAQRKALEKVRKDEYEIVGLAEWYVPAIVMYQAIHNNFHTKLEY |
| | | KSVSREEMIDYLENKTLSWNSKNPVSNGYWKRKKDDELKIIYNAIKLSEKEGNIFDIRDLYKRTLKNIDLLTYSFPCQD |
| | | LSQQGIQKGMKRGSGTRSGLLWEIERALDSTEKNDLPKYLLMENVGALLHKKNEEELNQWKQKLESLGYQNSIEVL |
| | | NAADFGSSQARRRVFMISTLNEFVELPKGDKKPKSIKKVLNKIVSEKDILNNLLKYNLTEFKKTKSNINKASLIGYSKF |
| | | NSEGYVYDPEFTGPTLTASGANSRIKIKDGSNIRKMNSDETFLYIGFDSQDGKRVNEIEFLTENQKIFVCGNSISVEVLE |
| | | AIIDKIGGPSSGGKRPAATKKAGQAKKKKGSYPYDVPDYAGSYPYDVPDYAATNFSLLKQAGDVEENPGPTSAGKL |
| | | GSGEGRGSLLTCGDVEENPGPLEGSSGSGSPKKKRKVGIHGVPAAGSSGSLEPGEKPYKCPECGKSFSRSDKLVRHQR |
| | | THTGEKPYKCPECGKSFSQRAHLERHQRTHTGEKPYKCPECGKSFSDPGHLVRHQRTHTGEKPYKCPECGKSFSRSD |
| | | KLVRHQRTHTGEKPYKCPECGKSFSQLAHLRAHQRTHTGEKPYKCPECGKSFSRADNLTEHQRTHTGEKPYKCPECG |
| | | KSFSDCRDLARHQRTHTGEKPTGKKTSASGSGGGSGGDAKSLTAWSRTLVTFKDVFVDFTREEWKLLDTAQQILYR |
| | | NVMLENYKNLVSLGYQLTKPDVILRLEKGEEPWLVEREIHQETHPDSETAFEIKSSVSGGKRPAATKKAGQAKKKKG |
| | | SYPYDVPDYA |
| ZF3- | 122 | MAPKKKRKVGIHGVPAAGSSGSLEPGEKPYKCPECGKSFSRSDKLVRHQRTHTGEKPYKCPECGKSFSQRAHLERHQ |
| KRAB + Z | | RTHTGEKPYKCPECGKSFSDPGHLVRHQRTHTGEKPYKCPECGKSFSRSDKLVRHQRTHTGEKPYKCPECGKSFSQL |
| F9- | | AHLRAHQRTHTGEKPYKCPECGKSFSRADNLTEHQRTHTGEKPYKCPECGKSFSDCRDLARHQRTHTGEKPTGKKTS |
| MQ1(aa) | | ASGSGGGSGGDAKSLTAWSRTLVTFKDVFVDFTREEWKLLDTAQQILYRNVMLENYKNLVSLGYQLTKPDVILRLE |
| | | KGEEPWLVEREIHQETHPDSETAFEIKSSVSSGGKRPAATKKAGQAKKKKGSYPYDVPDYAGSYPYDVPDYAATNFS |
| | | LLKQAGDVEENPGPTSAGKLGSGEGRGSLLTCGDVEENPGPLEGSSGSGSPKKKRKVGIHGVPAAGSSGSLEPGEKPY |
| | | KCPECGKSFSRSDDLVRHQRTHTGEKPYKCPECGKSFSREDNLHTHQRTHTGEKPYKCPECGKSFSRSDHLTTHQRT |
| | | HTGEKPYKCPECGKSFSQRANLRAHQRTHTGEKPYKCPECGKSFSQLAHLRAHQRTHTGEKPYKCPECGKSFSQRAN |
| | | LRAHQRTHTGEKPYKCPECGKSFSERSHLREHQRTHTGEKPTGKKTSASGSGGGSGGARDSKVENKTKKLRVFEAFA |
| | | GIGAQRKALEKVRKDEYEIVGLAEWYVPAIVMYQAIHNNFHTKLEYKSVSREEMIDYLENKTLSWNSKNPVSNGYW |
| | | KRKKDDELKIIYNAIKLSEKEGNIFDIRDLYKRTLKNIDLLTYSFPCQDLSQQGIQKGMKRGSGTRSGLLWEIERALDS |
| | | TEKNDLPKYLLMENVGALLHKKNEEELNQWKQKLESLGYQNSIEVLNAADFGSSQARRRVFMISTLNEFVELPKGD |
| | | KKPKSIKKVLNKIVSEKDILNNLLKYNLTEFKKTKSNINKASLIGYSKFNSEGYVYDPEFTGPTLTASGANSRIKIKDGS |
| | | NIRKMNSDETFLYIGFDSQDGKRVNEIEFLTENQKIFVCGNSISVEVLEAIIDKIGGPSGGKRPAATKKAGQAKKKKGS |
| | | YPYDVPDYA |
| ZF09- | 181 | MAPKKKRKVGIHGVPAAGSSGSLEPGEKPYKCPECGKSFSRSDDLVRHQRTHTGEKPYKCPECGKSFSREDNLHTHQ |
| MQ1- | | RTHTGEKPYKCPECGKSFSRSDHLTTHQRTHTGEKPYKCPECGKSFSQRANLRAHQRTHTGEKPYKCPECGKSFSQL |
| tPT2A- | | AHLRAHQRTHTGEKPYKCPECGKSFSQRANLRAHQRTHTGEKPYKCPECGKSFSERSHLREHQRTHTGEKPTGKKTS |
| ZF54- | | ASGSGGGSGGARDSKVENKTKKLRVFEAFAGIGAQRKALEKVRKDEYEIVGLAEWYVPAIVMYQAIHNNFHTKLEY |
| KRAB aa | | KSVSREEMIDYLENKTLSWNSKNPVSNGYWKRKKDDELKIIYNAIKLSEKEGNIFDIRDLYKRTLKNIDLLTYSFPCQD |
| | | LSQQGIQKGMKRGSGTRSGLLWEIERALDSTEKNDLPKYLLMENVGALLHKKNEEELNQWKQKLESLGYQNSIEVL |
| | | NAADFGSSQARRRVFMISTLNEFVELPKGDKKPKSIKKVLNKIVSEKDILNNLLKYNLTEFKKTKSNINKASLIGYSKF |
| | | NSEGYVYDPEFTGPTLTASGANSRIKIKDGSNIRKMNSDETFLYIGFDSQDGKRVNEIEFLTENQKIFVCGNSISVEVLE |
| | | AIIDKIGGPSGGKRPAATKKAGQAKKKKGSYPYDVPDYAGSATNFSLLKQAGDVEENPGPTSAGKLGSGEGRGSLLT |
| | | CGDVEENPGPLEGSSGSGSPKKKRKVGIHGVPAAGSSGSLEPGEKPYKCPECGKSFSERSHLREHQRTHTGEKPYKCP |
| | | ECGKSFSRSDNLVRHQRTHTGEKPYKCPECGKSFSDCRDLARHQRTHTGEKPYKCPECGKSFSTSGELVRHQRTHTG |
| | | EKPYKCPECGKSFSTTGNLTVHQRTHTGEKPYKCPECGKSFSRSDKLVRHQRTHTGEKPYKCPECGKSFSRTDTLRD |
| | | HQRTHTGKKTSASGSGGGSGGARDDAKSLTAWSRTLVTFKDVFVDFTREEWKLLDTAQQILYRNVMLENYKNLVS |
| | | LGYQLTKPDVILRLEKGEEPWLVEREIHQETHPDSETAFEIKSSVPSSGGKRPAATKKAGQAKKKKGSYPYDVPDYA |
| ZF54- | 182 | MAPKKKRKVGIHGVPAAGSSGSLEPGEKPYKCPECGKSFSERSHLREHQRTHTGEKPYKCPECGKSFSRSDNLVRHQ |
| KRAB- | | RTHTGEKPYKCPECGKSFSDCRDLARHQRTHTGEKPYKCPECGKSFSTSGELVRHQRTHTGEKPYKCPECGKSFSTTG |
| tPT2A- | | NLTVHQRTHTGEKPYKCPECGKSFSRSDKLVRHQRTHTGEKPYKCPECGKSFSRTDTLRDHQRTHTGKKTSASGSGG |
| ZF09- | | GSGGARDDAKSLTAWSRTLVTFKDVFVDFTREEWKLLDTAQQILYRNVMLENYKNLVSLGYQLTKPDVILRLEKGE |
| MQ1 aa | | EPWLVEREIHQETHPDSETAFEIKSSVPSSGGKRPAATKKAGQAKKKKGSYPYDVPDYAGSATNFSLLKQAGDVEEN |
| | | PGPTSAGKLGSGEGRGSLLTCGDVEENPGPLEGSSGSGSPKKKRKVGIHGVPAAGSSGSLEPGEKPYKCPECGKSFSRS |
| | | DDLVRHQRTHTGEKPYKCPECGKSFSREDNLHTHQRTHTGEKPYKCPECGKSFSRSDHLTTHQRTHTGEKPYKCPEC |
| | | GKSFSQRANLRAHQRTHTGEKPYKCPECGKSFSQLAHLRAHQRTHTGEKPYKCPECGKSFSQRANLRAHQRTHTGE |
| | | KPYKCPECGKSFSERSHLREHQRTHTGEKPTGKKTSASGSGGGSGGARDSKVENKTKKLRVFEAFAGIGAQRKALEK |
| | | VRKDEYEIVGLAEWYVPAIVMYQAIHNNFHTKLEYKSVSREEMIDYLENKTLSWNSKNPVSNGYWKRKKDDELKII |
| | | YNAIKLSEKEGNIFDIRDLYKRTLKNIDLLTYSFPCQDLSQQGIQKGMKRGSGTRSGLLWEIERALDSTEKNDLPKYLL |
| | | MENVGALLHKKNEEELNQWKQKLESLGYQNSIEVLNAADFGSSQARRRVFMISTLNEFVELPKGDKKPKSIKKVLN |
| | | KIVSEKDILNNLLKYNLTEFKKTKSNINKASLIGYSKFNSEGYVYDPEFTGPTLTASGANSRIKIKDGSNIRKMNSDETF |
| | | LYIGFDSQDGKRVNEIEFLTENQKIFVCGNSISVEVLEAIIDKIGGPSGGKRPAATKKAGQAKKKKGSYPYDVPDYA |
| ZF09- | 187 | MAPKKKRKVGIHGVPAAGSSGSLEPGEKPYKCPECGKSFSRSDDLVRHQRTHTGEKPYKCPECGKSFSREDNLHTHQ |
| MQ1- | | RTHTGEKPYKCPECGKSFSRSDHLTTHQRTHTGEKPYKCPECGKSFSQRANLRAHQRTHTGEKPYKCPECGKSFSQL |
| tPT2A- | | AHLRAHQRTHTGEKPYKCPECGKSFSQRANLRAHQRTHTGEKPYKCPECGKSFSERSHLREHQRTHTGEKPTGKKTS |
| ZF54- | | ASGSGGGSGGARDSKVENKTKKLRVFEAFAGIGAQRKALEKVRKDEYEIVGLAEWYVPAIVMYQAIHNNFHTKLEY |
| KRAB aa | | KSVSREEMIDYLENKTLSWNSKNPVSNGYWKRKKDDELKIIYNAIKLSEKEGNIFDIRDLYKRTLKNIDLLTYSFPCQD |
| without | | LSQQGIQKGMKRGSGTRSGLLWEIERALDSTEKNDLPKYLLMENVGALLHKKNEEELNQWKQKLESLGYQNSIEVL |
| HA tag | | NAADFGSSQARRRVFMISTLNEFVELPKGDKKPKSIKKVLNKIVSEKDILNNLLKYNLTEFKKTKSNINKASLIGYSKF |
| | | NSEGYVYDPEFTGPTLTASGANSRIKIKDGSNIRKMNSDETFLYIGFDSQDGKRVNEIEFLTENQKIFVCGNSISVEVLE |
| | | AIIDKIGGPSGGKRPAATKKAGQAKKKKGSYPYDVPDYAGSATNFSLLKQAGDVEENPGPTSAGKLGSGEGRGSLLT |
| | | CGDVEENPGPLEGSSGSGSPKKKRKVGIHGVPAAGSSGSLEPGEKPYKCPECGKSFSERSHLREHQRTHTGEKPYKCP |
| | | ECGKSFSRSDNLVRHQRTHTGEKPYKCPECGKSFSDCRDLARHQRTHTGEKPYKCPECGKSFSTSGELVRHQRTHTG |
| | | EKPYKCPECGKSFSTTGNLTVHQRTHTGEKPYKCPECGKSFSRSDKLVRHQRTHTGEKPYKCPECGKSFSRTDTLRD |
| | | HQRTHTGKKTSASGSGGGSGGARDDAKSLTAWSRTLVTFKDVFVDFTREEWKLLDTAQQILYRNVMLENYKNLVS |
| | | LGYQLTKPDVILRLEKGEEPWLVEREIHQETHPDSETAFEIKSSVPSSGGKRPAATKKAGQAKKKKGS |
| ZF54- | 188 | MAPKKKRKVGIHGVPAAGSSGSLEPGEKPYKCPECGKSFSERSHLREHQRTHTGEKPYKCPECGKSFSRSDNLVRHQ |
| KRAB- | | RTHTGEKPYKCPECGKSFSDCRDLARHQRTHTGEKPYKCPECGKSFSTSGELVRHQRTHTGEKPYKCPECGKSFSTTG |
| tPT2A- | | NLTVHQRTHTGEKPYKCPECGKSFSRSDKLVRHQRTHTGEKPYKCPECGKSFSRTDTLRDHQRTHTGKKTSASGSGG |
| ZF09- | | GSGGARDDAKSLTAWSRTLVTFKDVFVDFTREEWKLLDTAQQILYRNVMLENYKNLVSLGYQLTKPDVILRLEKGE |
| MQ1 aa | | EPWLVEREIHQETHPDSETAFEIKSSVPSSGGKRPAATKKAGQAKKKKGSYPYDVPDYAGSATNFSLLKQAGDVEEN |
| without | | PGPTSAGKLGSGEGRGSLLTCGDVEENPGPLEGSSGSGSPKKKRKVGIHGVPAAGSSGSLEPGEKPYKCPECGKSFSRS |
| HA tag | | DDLVRHQRTHTGEKPYKCPECGKSFSREDNLHTHQRTHTGEKPYKCPECGKSFSRSDHLTTHQRTHTGEKPYKCPEC |
| | | GKSFSQRANLRAHQRTHTGEKPYKCPECGKSFSQLAHLRAHQRTHTGEKPYKCPECGKSFSQRANLRAHQRTHTGE |
| | | KPYKCPECGKSFSERSHLREHQRTHTGEKPTGKKTSASGSGGGSGGARDSKVENKTKKLRVFEAFAGIGAQRKALEK |
| | | VRKDEYEIVGLAEWYVPAIVMYQAIHNNFHTKLEYKSVSREEMIDYLENKTLSWNSKNPVSNGYWKRKKDDELKII |
| | | YNAIKLSEKEGNIFDIRDLYKRTLKNIDLLTYSFPCQDLSQQGIQKGMKRGSGTRSGLLWEIERALDSTEKNDLPKYLL |
| | | MENVGALLHKKNEEELNQWKQKLESLGYQNSIEVLNAADFGSSQARRRVFMISTLNEFVELPKGDKKPKSIKKVLN |
| | | KIVSEKDILNNLLKYNLTEFKKTKSNINKASLIGYSKFNSEGYVYDPEFTGPTLTASGANSRIKIKDGSNIRKMNSDETF |
| | | LYIGFDSQDOKRVNEIEFLTENQKITVCGNSISVEVLLAIIDKIGGPSGGKRPAATKKAGQAKKKKGS |

In some embodiments, an expression repressor comprises a nuclear localization sequence (NLS). In some embodiments, the expression repressor comprises an NLS, e.g., an SV40 NLS at the N-terminus. In some embodiments, the expression repressor comprises an NLS, e.g., a nucleoplasmin NLS at the C-terminus. In some embodiments, the expression repressor comprises a first NLS at the N-terminus and a second NLS at the C-terminus. In some embodiments the first and the second NLS have the same sequence. In some embodiments, the first and the second NLS have different sequences. In some embodiments, the expression repression repressor comprises an SV40 NLS, e.g., the expression repressor comprises a sequence according to PICKKRK. (SEQ ID NO: 135). In some embodiments, the N-terminal sequence comprises an NLS and a spacer, e.g., having a sequence according to: MAPKKKRKVGIHGVPAAGSSGS (SEQ ID NO: 88). In some embodiments, the expression repressor comprises a C-terminal sequence comprising one or more of, e.g., any two or all three of: a spacer, a nucleoplasmin nuclear localization sequence and an HA-tag: e.g., SGGKRPAATKKAGQAKKKGSYPYDVPDYA (SEQ ID NO: 89). In some embodiments, the expression repressor comprises an epitope tag, e.g., an HA tag: YPYDVPDYA (SEQ ID NO: 90). For example, the expression repressor may comprise two copies of the epitope tag.

While an epitope tag is useful in many research contexts, it is sometimes desirable to omit an epitope tag in a therapeutic context. Accordingly, in some embodiments, the expression repressor lacks an epitope tag. In some embodiments, an expression repressor described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the HA tag of SEQ ID NO: 90. In some embodiments, a nucleic acid described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking a region encoding the HA tag of SEQ ID NO: 90. In some embodiments, the expression repressor comprises a nucleoplasmin NLS, e.g., the expression repressor comprises a sequence of KRPAATKKAGQAKKK (SEQ ID NO: 136). In some embodiments, the expression repressor does not comprise an NLS. In some embodiments, the expression repressor does not comprise an epitope tag. In some embodiments the expression repressor does not comprise an HA tag. In some embodiments, the expression repressor does not comprise an HA tag sequence according to SEQ ID NO: 90.

In some embodiments, the present disclosure provides an expression repressor system comprises a self-cleaving peptide. Self-cleaving peptides, first discovered in picornaviruses, are peptides of between 19 to 22 amino acids in length and are usually found between two proteins in some members of the picornavirus family. Using self-cleaving proteins, picornaviruses are capable of producing equimolar levels of multiple genes from the same mRNA. Such self-cleaving proteins are known to be found in other species of viruses and a person skilled in the art, based on the information provided herein, will be readily able to determine a suitable substitution for the self-cleaving protein disclosed herein, if required. In some embodiments, an expression repressor system comprises a self-cleaving peptide, e.g., a 2A self-cleaving peptide. In some embodiments, the 2A peptide comprises a single cleavage site, e.g., a 2A peptide, e.g., a P2A, a T2A, a E2A, or a F2A peptide. In some embodiments the self-cleaving peptide, e.g., a 2A peptide, comprises two cleavage sites, e.g., pPT2A, or P2A-T2A-E2A. In some embodiments, an expression repressor system comprises a self-cleaving peptide comprising a plurality of cleavage sites, e.g., a T2A self-cleaving peptide and a P2A self-cleaving peptide. In some embodiments, the 2A peptide gets cleaved after translation. In some embodiments, the self-cleaving peptide produces two or more fragments after cleaving. In some embodiments, the 2A peptide fragments comprise the sequences of SEQ ID NO: 126-128. In some embodiments, the 2A self-cleaving peptide comprises a sequence of SEQ ID NO: 120, 124, 125 or derivative thereof. In some embodiments, SEQ ID NO: 95 comprises a sequence of a self-cleaving peptide.

(SEQ ID NO: 95)
PSSGGKRPAATKKAGQAKKKKGSYPYDVPDYAGSYPYDVPDYAATNFSLL
KQAGDVEENPGPTSAGKLGSGEGRGSLLTCGDVEENPGPLEGSSGSGSPK
KKRKVGIHGVPAAGSSGS
(SEQ ID NO: 120)
EGRGSLLTCGDVEENPGP
(SEQ ID NO: 124)
ATNFSLLKQAGDVEENPGPTSAGKLGSGEGRGSLLTCGDVEENPGP
(SEQ ID NO: 125)
ATNFSLLKQAGDVEENPGP
(SEQ ID NO: 126)
ATNFSLLKQAGDVEENPG
(SEQ ID NO: 127)
PTSAGKLGSGEGRGSLLTCGDVEENPG
(SEQ ID NO: 128)
P

It is of course understood that although a 2A sequence, e.g., tPT2A sequence (e.g., according to SEQ ID NO: 124), may be referred to in the scientific literature and herein as a self-cleaving peptide, this is according to a non-limiting theory. According to another non-limiting theory, in some embodiments, a 2A sequence acts via ribosome-skipping. For instance, an mRNA encoding a 2A sequence may induce ribosome skipping, wherein the ribosome fails to form a peptide bond while translating the 2A region, resulting in a release of the first part of the translation product. The ribosome then produces the second part of the translation product. Overall, it is well established that a 2A sequence placed between a first sequence and a second sequence will lead to the production of a first protein comprising the first sequence and a separate, second protein comprising the second sequence. This disclosure is not bound by any particular theory as to the molecular mechanism by which this is achieved.

Functional Characteristics

An expression repressor or a system of the present disclosure can be used to decrease expression of a target gene, e.g., MYC, in a cell. In general, an expression repressor or a system as described herein binds (e.g., via a targeting moiety) a genomic sequence element proximal to and/or operably linked to a target gene, e.g., MYC. In some embodiments, binding of the expression repressor or the system to the genomic sequence element modulates (e.g., decreases) expression of the target gene, e.g., MYC. For example, binding of an expression repressor or a system comprising an effector moiety that inhibits recruitment of components of the transcription machinery to the genomic sequence element may modulate (e.g., decrease) expression of the target gene, e.g., MYC. As a further example, binding of an expression repressor or a system comprising an effector moiety with an enzymatic activity (e.g., an epigenetic modifying moiety) may modulate (e.g., decrease) expression of the target gene, e.g., MYC) through the localized enzymatic activity of the effector moiety. As a further example, both binding of an expression repressor or a system to a genomic sequence element and the localized enzymatic activity of an expression repressor or a system may contribute to the resulting modulation (e.g., decrease) in expression of the target gene, e.g., MYC.

In some embodiments, decreasing expression comprises decreasing the level of RNA, e.g., mRNA, encoded by the target gene e.g., MYC. In some embodiments, decreasing expression comprises decreasing the level of a protein encoded by the target gene e.g., MYC. In some embodiments, decreasing expression comprises both decreasing the level of mRNA and protein encoded by the target gene e.g., MYC. In some embodiments, the expression of a target gene in a cell contacted by or comprising the expression repressor or the expression repression system disclosed herein is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× lower than the level of expression of the target gene in a cell not contacted by or comprising the expression repressor or the expression repression system disclosed herein. Expression of a target gene e.g., MYC may be assayed by methods known to those of skill in the art, including RT-PCR, ELISA, Western blot, and the methods of Examples 2-9. Expression level of a target gene, e.g., MYC in a subject, e.g., a patient, e.g., a patient having a MYC mis-regulation disorder, e.g., a patient having a hepatic disease, a patient having a neoplasia and/or viral or alcohol related hepatic disease, e.g., a patient having a hepatocarcinoma, e.g., a patient having a hepatocarcinoma subtype S1 or hepatocarcinoma subtype S2, may be assessed by evaluating blood (e.g., whole blood) levels of the target gene, e.g., MYC, e.g., by the method of either Oglesbee et al. Clin Chem. 2013 October; 59(10):1461-9. Doi: 10.1373/clinchem.2013.207472 or Deutsch et al. J Neurol Neurosurg Psychiatry. 2014 September; 85(9):994-1002. Doi: 10.1136/jnnp-2013-306788, the contents of which are hereby incorporated by reference in their entirety.

An expression repressor or a system of the present disclosure can be used to decrease expression of a target gene e.g., MYC in a cell for a time period. In some embodiments, the expression of a target gene e.g., MYC in a cell contacted by or comprising the expression repressor or the system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, 14, or 15 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the expression of a target gene, e.g., MYC in a cell contacted by or comprising the expression repressor or the system is appreciably decreased for no more than 10, 9, 8, 7, 6, 5, 4, 3, 2, or 1 years. In some embodiments, the expression of a target gene e.g., MYC in a cell contacted by or comprising the expression repressor or the system is appreciably decreased for at least 1, 2, 3, 4, 5, 6, 7, 8, 9, or 10 cell divisions. An expression repressor or a system of the present disclosure can be used to methylate CpG nucleotides in a target promoter, e.g., MYC promoter. In some embodiments, the transcriptional changes in MYC expression correlates to percentage of CpG methylation. In some embodiments, the methylation persists for at least 1 days, at least 2 days, at least 5 days, at least 7 days, at least 10 days, at least 15 days, or at least 20 days post-treatment with an expression repressor or a system disclosed herein.

An expression repressor or a system of the present disclosure can be used to decrease the viability of a cell comprising the target locus, e.g., MYC locus. In some embodiments, expression repressor or a system of the present disclosure can be used to decrease the viability of a plurality of cells comprising the target locus, e.g., MYC locus. In some embodiments, the number of viable cells contacted by or comprising the expression repressor, or the system is appreciably decreased by 10, 20, 30, 40, 50, 60, 70, 80, 90, or 100% compared to number of viable cells in a control population of cells that is not contacted by or does not comprise the expression repressor or the system.

In some embodiments, an expression repressor or a system of the present disclosure can be used to decrease the viability of a plurality of cells comprising cancer cells and non-cancer cells. In some embodiments, an expression repressor or a system of the present disclosure can be used to decrease the viability of the plurality of cancer cells more than it decreases the viability of the plurality of non-cancer cells. In some embodiments, an expression repressor or a system of the present disclosure can be used to decrease the viability of the plurality of cancer cells 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than it decreases the viability of the plurality of non-cancer cells.

In some embodiments, an expression repressor or a system of the present disclosure can be used to decrease the viability of a plurality of cells comprising infected cells and uninfected cells. In some embodiments, an expression repressor or a system of the present disclosure can be used to decrease the viability of the plurality of infected cells more than it decreases the viability of the plurality of uninfected cells. In some embodiments, an expression repressor or a system of the present disclosure can be used to decrease the viability of the plurality of infected cells 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than it decreases the viability of the plurality of uninfected cells.

An expression repression system may comprise a plurality of expression repressors, where each expression repressor comprises an effector moiety with a different functionality than the effector moiety of another expression repressor. For example, an expression repression system may comprise two expression repressors, where the first expression repressor comprises a first effector moiety comprising an epigenetic modifying moiety e.g., DNA methyltransferase, e.g., MQ1 and the second expression repressor comprises a second effector moiety comprising a transcription repressor, e.g., KRAB. In some embodiments, the second expression repressor does not comprise a second effector moiety. In some embodiments, an expression repression system comprises expression repressors comprising a combination of effector moieties whose functionalities are complementary to one another with regard to inhibiting expression of a target gene, e.g., MYC, where the functionalities together enable inhibition of expression and, optionally, do not inhibit or negligibly inhibit expression when present individually. In some embodiments, an expression repression system comprises a plurality of expression repressors, wherein each expression repressor comprises an effector moiety that complements the effector moieties of each other expression repressor, e.g., each effector moiety decreases expression of a target gene, e.g., MYC.

In some embodiments, an expression repression system comprises expression repressors comprising a combination of effector moieties whose functionalities synergize with one another with regards to inhibiting expression of a target gene, e.g., MYC. Without wishing to be bound by theory, epigenetic modifications to a genomic locus may be cumulative, in that multiple repressive epigenetic markers (e.g., multiple different types of epigenetic markers and/or more extensive marking of a given type) individually together reduce expression more effectively than individual modifications alone (e.g., producing a greater decrease in expression and/or a longer-lasting decrease in expression). In some embodiments, an expression repression system comprises a plurality of expression repressors, wherein each expression repressor comprises an effector moiety that synergizes with the effector moieties of each other expression repressor, e.g., each effector moiety decreases expression of a target gene, e.g., MYC.

In some embodiments, an expression repressor or a system modulates (e.g., decreases) expression of a target gene, e.g., MYC by altering one or more epigenetic markers associated with the target gene, e.g., MYC or an expression control sequence operably linked thereto. In some embodiments, altering comprises decreasing the level of an epigenetic marker associated with the target gene, e.g., MYC or an expression control sequence operably linked thereto. Epigenetic markers include, but are not limited to, DNA methylation, histone methylation, and histone deacetylation.

In some embodiments, altering the level of an epigenetic marker decreases the level of the epigenetic marker associated with the target gene, e.g., MYC or an expression control sequence operably linked thereto by at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× lower than the level of the epigenetic marker associated with the target gene, e.g., MYC or an expression control sequence operably linked thereto in a cell not contacted by or comprising the expression repressor or the system. The level of an epigenetic marker may be assayed by methods known to those of skill in the art, including whole genome bisulfite sequencing, reduced representation bisulfite sequencing, bisulfite amplicon sequencing, methylation arrays, pyrosequencing, ChIP-seq, or ChIP-qPCR. In some embodiments, the changes (e.g., increase or decrease) in epigenetic marker e.g., DNA methylation may be assayed using bisulfite genomic sequencing at precise genomic coordinates according to hg19 reference genome, e.g., in between chr8:129188693-129189048 according to hg19 reference genome. In some embodiments, the changes (e.g., increase or decrease) in epigenetic marker e.g., DNA methylation may be assayed using bisulfite genomic sequencing at a genomic location according to SEQ ID NO: 123.

(SEQ ID NO: 123)
CAGAGAAGGAGGAAGTTAATTCACATTCTTAATTTTTTCTAAGGGCAAAA
AAAAAAAAAAAATGCACCAGCTCATTTTCCATCTCTGCTTGGGTCATCAG
TGTGCATTGTGAGCCTGTACAAAGGCCTTAGACGGGGAATGCTGCCGAGA
GCATCACCTTTTATGTCTTCTTTTATATGAAATGTGCCACTTCCCCACTA
ACCCTGGCTCTGGGCTCTGCCTCTGCTCTCCTGATGGTGTGTTTATGGTG
GATTCAGCATTCTGGGCCACACAAGGAAGCTGCAGGGGGTGTCCAAGTTC
ACATGTCCCCGCATTCCAGGCGAATGTTTCTGACATTGAGCAATGATATG
GCTCT

An expression repressor or the system of the present disclosure can be used to alter the level of an epigenetic marker associated with the target gene, e.g., MYC or an expression control sequence operably linked thereto in a cell for a time period. In some embodiments, the level of the epigenetic marker associated with the target gene or an expression control sequence operably linked thereto in a cell contacted by or comprising the expression repressor or the system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the level of an epigenetic marker associated with the target gene, e.g., MYC or an expression control sequence operably linked thereto in a cell contacted by or comprising the expression repressor or the system is appreciably decreased for no more than 10, 9, 8, 7, 6, 5, 4, 3, 2, or 1 years.

Combinations of Repressors

In some embodiments, an expression repression system comprises a first expression repressor comprising a first effector moiety and a second expression repressor comprising a second effector moiety wherein the first effector moiety and second effector moiety are different from one another. In some embodiments, the first effector moiety is or comprises a first epigenetic modifying moiety (e.g., that increases or decreases a first epigenetic marker) or functional fragment thereof and the second effector moiety is or comprises a second epigenetic modifying moiety (e.g., that increases or decreases a second epigenetic marker) or functional fragment thereof. In some embodiments, the first effector moiety is or comprises a DNA methyltransferase or functional fragment thereof and the second effector moiety is or comprises a KRAB or functional fragment thereof. In some embodiments, the first effector moiety is or comprises a histone deacetylase or functional fragment thereof and the second effector moiety is or comprises a KRAB or functional fragment thereof. In some embodiments, the first effector moiety is or comprises a histone methyltransferase or functional fragment thereof and the second effector moiety n is or comprises a KRAB or functional fragment thereof. In some embodiments, the first effector moiety is or comprises a histone demethylase or functional fragment thereof and the second effector moiety is or comprises a KRAB or functional fragment thereof.

In some embodiments, the first effector moiety is or comprises MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, MACS, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2 or a functional fragment of any thereof, and the second effector moiety is or comprises KRAB (e.g., a KRAB domain), MeCP2, HP1, RBBP4, REST, FOG1, SUZ12, or a functional fragment of any thereof.

In some embodiments, the first effector moiety is or comprises KRAB (e.g., a KRAB domain), MeCP2, HP1, RBBP4, REST, FOG1, SUZ12, or a functional fragment of any thereof, and the second effector moiety is or comprises MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2 or a functional fragment of any thereof.

In some embodiments, the first effector moiety is or comprises bacterial MQ1 or a functional variant or fragment thereof, and the second effector moiety is or comprises KRAB or a functional variant or fragment thereof.

In some embodiments, the first effector moiety is or comprises DNMT3A or a functional variant or fragment thereof, and the second effector moiety is or comprises KRAB or a functional variant or fragment thereof.

In some embodiments, the first effector moiety is or comprises DNMT3B or a functional variant or fragment thereof, and the second effector moiety is or comprises KRAB or a functional variant or fragment thereof.

In some embodiments, the first effector moiety is or comprises DNMT3L or a functional variant or fragment thereof, and the second effector moiety is or comprises KRAB or a functional variant or fragment thereof.

In some embodiments, the first effector moiety is or comprises DNMT3a/3L complex or a functional variant or fragment thereof, and the second effector moiety is or comprises KRAB or a functional variant or fragment thereof.

### Target Sites

Expression repressors or expression repressor systems disclosed herein are useful for modulating, e.g., decreasing, expression of a target gene, e.g., MYC in cell, e.g., in a subject or patient. A target gene, e.g., MYC may be any gene known to those of skill in the art. In some embodiments, a target gene, e.g., MYC is associated with a disease or condition in a subject, e.g., a mammal, e.g., a human, bovine, horse, sheep, chicken, rat, mouse, cat, or dog. A target gene may include coding sequences, e.g., exons, and/or non-coding sequences, e.g., introns, 3′UTR, or 5′UTR. In some embodiments, a target gene is operably linked to a transcription control element.

A targeting moiety suitable for use in an expression repressor or an expression repressor of system described herein may bind, e.g., specifically bind, to any site within a target gene, e.g., MYC, transcription control element operably linked to a target gene, e.g., MYC to an anchor sequence (e.g., an anchor sequence proximal to a target gene or associated with an anchor sequence-mediated conjunction operably linked to a target gene, e.g., MYC (e.g., an anchor sequence-mediated conjunction is operably linked to a target gene if disruption of the conjunction alters expression of the target gene, e.g., MYC)), or to a regulatory element located in a super enhancer region (e.g., a regulatory element located in a super enhancer region of MYC).

In some embodiments, an expression repressor described herein binds at a site or at a location that is proximal to the site. For example, a targeting moiety may bind to a first site that is proximal to a repressor (the second site), and the effector moiety associated with said targeting moiety may epigenetically modify the first site such that the enhancer's effect on expression of a target gene is modified, substantially the same as if the second site (the enhancer sequence) had been bound and/or modified. In some embodiments, a site proximal to a target gene (e.g., an exon, intron, or splice site within the target gene), proximal to a transcription control element operably linked to the target gene, e.g., MYC, or proximal to an anchor sequence is less than 5000, 4000, 3000, 2000, 1000, 900, 800, 700, 600, 500, 400, 300, 200, 100, 50, or 25 base pairs from the target gene, e.g., MYC (e.g., an exon, intron, or splice site within the target gene, e.g., MYC), transcription control element, or anchor sequence (and optionally at least 20, 25, 50, 100, 200, or 300 base pairs from the target gene, e.g., MYC (e.g., an exon, intron, or splice site within the target gene), transcription control element, or anchor sequence).

In some embodiments, a targeting moiety binds to a target gene, e.g., MYC. In some embodiments, a DNA-targeting moiety binds to a site within an exon of a target gene, e.g., MYC. In some embodiments, a targeting moiety binds to a site within an intron of a target gene, e.g., MYC. In some embodiments, a targeting moiety binds to a site at the boundary of an exon and an intron, e.g., a splice site, of a target gene, e.g., MYC. In some embodiments, a targeting moiety binds to a site within the 5′UTR of a target gene, e.g., MYC. In some embodiments, a targeting moiety binds to a site within the 3′UTR of a target gene, e.g., MYC. Target genes include, but are not limited to the gene encoding MYC.

In some embodiments, a targeting moiety binds to a transcription control element operably linked to a target gene (e.g., MYC), e.g., a promoter or enhancer. In some embodiments, a targeting moiety binds to a portion of or a site within a promoter operably linked to a target gene, e.g., MYC. In some embodiments, a targeting moiety binds to the transcription start site of a target gene, e.g., MYC. In some embodiments, a targeting moiety binds to a portion of or a site within an enhancer operably linked to a target gene, e.g., MYC. In some embodiments, a genomic complex (e.g., ASMC) co-localizes two or more genomic sequence elements, wherein the two or more genomic sequence elements include a promoter. A promoter is, typically, a sequence element that initiates transcription of an associated gene. Promoters are typically near the 5′ end of a gene, not far from its transcription start site. As those of ordinary skill are aware, transcription of protein-coding genes in eukaryotic cells is typically initiated by binding of general transcription factors (e.g., TFIID, TFIIE, TFIIH, FUSE, CT-element etc.) and Mediator to core promoter sequences as a preinitiation complex that directs RNA polymerase II to the transcription start site, and in many instances remains bound to the core promoter sequences even after RNA polymerase escapes and elongation of the primary transcript is initiated. In some embodiments, a promoter includes a sequence element such as TATA, Inr, DPE, or BRE, but those skilled in the art are well aware that such sequences are not necessarily required to define a promoter. Those skilled in the art are familiar with a variety of positive (e.g., enhancers) or negative (e.g., repressors or silencers) transcription control elements that are associated with genes. In some embodiments, a transcription control element is a transcription factor binding site. Typically, when a cognate regulatory protein is bound to such a transcription control element, transcription from the associated gene(s) is altered (e.g., increased or decreased). In some embodiments, a targeting moiety binds to a genomic sequence located within a genomic coordinate GRCh37: chr8:129162465-129212140.

In some embodiments, a targeting moiety binds to a target sequence comprised by or partially comprised by a genomic sequence element. In some embodiments, the genomic sequence element is or comprises an expression control sequence. In some embodiments, the genomic sequence element is or comprises the target gene, e.g., MYC or a part of the target gene, e.g., MYC. In some embodiments, a targeting moiety binds to a target sequence that is at least 10, 11, 12, 13, 14, 15, 16, 17, 18, 19, 20, 21, 22, 23, 24, 25, 26, 27, 28, 29, 30, 31, 32, 33, 34, or 35 bases long (and optionally no more than 40, 39, 38, 37, 36, 35, 34, 33, 32, 31, 30, 29, 28, 27, 26, 25, 24, 23, 22, 21, or 20 bases long). In some embodiments, a targeting moiety binds to a target sequence that is 10-30, 15-30, 15-25, 18-24, 19-23, 20-23, 21-23, or 22-23 bases long. In some embodiments, the target sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases long. In some embodiments, the genomic sequence element is or comprises an anchor sequence.

Each ASMC comprises one or more anchor sequences, e.g., a plurality. In some embodiments, anchor sequences can be manipulated or altered to modulate (e.g., disrupt) a naturally occurring genomic complex (e.g., ASMC) or to form a new genomic complex (e.g., ASMC) (e.g., to form a non-naturally occurring genomic complex (e.g., ASMC) with an exogenous or altered anchor sequence). In some embodiments, an anchor sequence-mediated conjunction can be disrupted to alter, e.g., inhibit, e.g., decrease expression of a target gene. Such disruptions may modulate gene expression by, e.g., changing topological structure of DNA, e.g., by modulating the ability of a target gene to interact with a transcription control element (e.g., enhancing and silencing/repressive sequences).

In some embodiments, a targeting moiety binds to an anchor sequence, e.g., an anchor sequence proximal to a target gene, e.g., MYC or associated with an anchor sequence-mediated conjunction (ASMC) operably linked to a target gene, e.g., MYC (e.g., an anchor sequence-mediated conjunction is operably linked to a target gene, e.g., MYC if disruption of the conjunction alters expression of the target gene, e.g., MYC). In general, an anchor sequence is a genomic sequence element to which a genomic complex component, e.g., nucleating polypeptide binds specifically. In some embodiments, binding of a genomic complex component to an anchor sequence nucleates complex formation, e.g., ASMC formation. In some embodiments, a targeting moiety binds to a target gene, e.g., MYC locus. A locus is generally defined to encompass transcribed region, promoter, and anchor sites of an ASMC comprising a target gene, e.g., MYC. In some embodiments, a targeting moiety binds to a sequence comprising any one of SEQ ID NOS: 75-86 or 199-206. In some embodiments, the first targeting moiety binds to a sequence comprising any one of SEQ ID NOS: 75-86 and the second targeting moiety binds to a sequence comprising any one of SEQ ID NOS: 75-86, wherein the first and the second targeting moiety binds to the same sequence. In some embodiments, the first targeting moiety binds to a sequence comprising any one of SEQ ID NOS: 75-86 and the second targeting moiety binds to a sequence comprising any one of SEQ ID NOS: 75-86 wherein the first and the second targeting moiety binds to different sequences. In some embodiments, the first targeting moiety binds to a sequence comprising any of SEQ ID NOs: 83, 203, or 206 and the second targeting moiety binds to a sequence comprising SEQ ID NO:77. In some embodiments, the first targeting moiety binds to a sequence comprising SEQ ID NO: 77 and the second targeting moiety binds to a sequence comprising any of SEQ ID NOs:83, 203, or 206. In some embodiments, the first targeting moiety binds to a sequence comprising any of SEQ ID NOs: 83, 203, or 206 and the second targeting moiety binds to a sequence comprising any of SEQ ID NOs:199, 204, or 205. In some embodiments, the first targeting moiety binds to a sequence comprising any of SEQ ID NOs: 199, 204, or 205 and the second targeting moiety binds to a sequence comprising any of SEQ ID NOs:83, 203, or 206. In some embodiments, the first targeting moiety binds to a sequence comprising any of SEQ ID NOs: 83, 203, or 206 and the second targeting moiety binds to a sequence comprising SEQ ID NO:201. In some embodiments, a nucleic acid encoding the first and second expression repressors comprises a first region that encodes the first expression repressor, wherein the first region is upstream of a second region that encodes the second expression repressor. In some embodiments, a nucleic acid encoding the first and second expression repressors comprises a first region that encodes the first expression repressor, wherein the first region is downstream of a second region that encodes the second expression repressor. In some embodiments, the first targeting moiety binds to a sequence comprising any one of SEQ ID NOs: 75-86 or 199-206, and the second targeting moiety (e.g., a CRISPR/Cas domain comprising a gRNA) binds to a sequence comprising any one of SEQ ID NOS: 1-4. In some embodiments, a targeting moiety binds to a sequence comprising any one of SEQ ID NOS: 96-110. In some embodiments, the first targeting moiety binds to a sequence comprising any one of SEQ ID NOS: 96-110 and the second targeting moiety binds to a sequence comprising any one of SEQ ID NOS: 96-110, wherein the first and the second targeting moiety binds to the same sequence. In some embodiments, the first targeting moiety binds to a sequence comprising any one of SEQ ID NOS: 96-110 and the second targeting moiety binds to a sequence comprising any one of SEQ ID NOS: 96-110 wherein the first and the second targeting moiety binds to different sequences. In some embodiments, the first targeting moiety binds to a sequence comprising any one of SEQ ID NOs: 96-110, and the second targeting moiety (e.g., a CRISPR/Cas domain comprising a gRNA) binds to a sequence comprising any one of SEQ ID NOS: 1-4. In some embodiments, the first targeting moiety binds to a sequence comprising any one of the SEQ ID Nos. disclosed in tables 2, 12, or 13, and the second targeting moiety (e.g., a CRISPR/Cas domain comprising a gRNA) binds to a sequence comprising any one of the SEQ ID Nos. disclosed in tables 2, 12, or 11 Exemplary target sequences are disclosed in Table 12.

TABLE 12
Exemplary target sequences

| | SEQ | | |
|---|---|---|---|
| | ID | | GENOMIC |
| NAME | NO: | SEQUENCE | COORDINATES |
| ZF1- | 75 | GCTGGAAACCTTGCACCTCGG | GRCh37: |
| KRAB | | | chr8:128746267- |
| | | | 128746287 |
| ZF2- | 76 | CTGCTGCCAGTAGAGGGCACA | GRCh37: |
| KRAB | | | chr8:128746349- |
| | | | 128746369 |
| ZF3- | 77 | GCCCAGAGAGGGGGCGGAGGG | GRCh37: |
| KRAB | | | chr8:128746405- |
| | | | 128746425 |
| ZF4- | 78 | ACGCGGGGAGCAACCAATCGC | GRCh37: |
| KRAB | | | chr8:128746455- |
| | | | 128746475 |
| ZF5- | 79 | ACTGGCAGCAGAGATCATCGC | GRCh37: |
| KRAB | | | chr8:128746339- |
| | | | 128746359 |
| ZF6- | 80 | GGGGGCAGGAGCAGGAGCGTC | GRCh37: |
| KRAB | | | chr8:128746287- |
| | | | 128746307 |
| ZF7-MQ1 | 81 | CAGCCTTAGCGAGGCGCCCTG | GRCh37: |
| | | | chr8:128747885- |
| | | | 128747905 |
| ZF8-MQ1 | 82 | ACTCACAGGACAAGGATGCGG | GRCh37: |
| | | | chr8:128747990- |
| | | | 128748010 |
| ZF9-MQ1 | 83 | AGCAAAAGAAAATGGTAGGCG | GRCh37: |
| | | | chr8:128748069- |
| | | | 128748089 |
| ZF10- | 84 | ACTCAGCCGGGCAGCCGAGCA | GRCh37: |
| MQ1 | | | chr8:128748143- |
| | | | 128748163 |
| ZF11- | 85 | CGTACCAGGCTGCAGGGCGCC | GRCh37: |
| MQ1 | | | chr8:128747897- |
| | | | 128747917 |
| ZF12- | 86 | AGAGTGGAGGAAAGAAGGGTA | GRCh37: |
| MQ1 | | | chr8:128747829- |
| | | | 128747849 |
| ZF54- | 199 | ACGGGGAATGCTGCCGAGAGC | GRCh37: |
| KRAB | | | chr8:129188825- |
| | | | 129188845 |
| ZF61- | 200 | ACCTGAACCCTGGAAATTATA | GRCh37: |
| KRAB | | | chr8:129209866- |
| | | | 129209888 |
| ZF67- | 201 | TAGACGGGGAATGCTGCCGAG | GRCh37: |
| KRAB | | | chr8:129188822- |
| | | | 129188842 |
| ZF68- | 202 | TGACATTGAGCAATGATATGG | GRCh37: |
| KRAB | | | chr8:129189024- |
| | | | 129189044 |
| ZF09- | 203 | AGCAAAAGAAAATGGTAGGCG | GRCh37: |
| MQ1- | | | chr8:128748069- |
| tPT2A- | | | 128748089 |
| ZF54- | | | |
| KRAB | | | |
| target | | | |
| se- | | | |
| quence 1 | | | |
| ZF09- | 204 | ACGGGGAATGCTGCCGAGAGC | GRCh37: |
| MQ1- | | | chr8:129188825- |
| tPT2A- | | | 129188845 |
| ZF54- | | | |
| KRAB | | | |
| target | | | |
| se- | | | |
| quence 2 | | | |
| ZF54- | 205 | ACGGGGAATGCTGCCGAGAGC | GRCh37: |
| KRAB- | | | chr8:129188825- |
| tPT2A- | | | 129188845 |
| ZF09- | | | |
| MQ1 | | | |
| target | | | |
| se- | | | |
| quence 1 | | | |
| ZF54- | 206 | AGCAAAAGAAAATGGTAGGCG | GRCh37: |
| KRAB- | | | chr8:128748069- |
| tPT2A- | | | 128748089 |
| ZF09- | | | |
| MQ1 | | | |
| target | | | |
| se- | | | |
| quence 2 | | | |

In some embodiments, an expression repressor binds a genomic locus having a sequence set forth herein, e.g., any one of SEQ ID NOS: 1-4, 75-86, 96-110, or 199-206. It is understood that, in many cases, the genomic locus being bound comprises double stranded DNA, and this locus can be described by giving the sequence of its sense strand or its antisense strand. Thus, a gRNA having a given spacer sequence may cause expression repressor to bind to a particular genomic locus, wherein one strand of the genomic locus has a sequence similar or identical to the spacer sequence, and the other strand of the genomic locus has the complementary sequence. Typically, gRNA binding to the genomic locus will involve some unwinding of the genomic locus and pairing of the gRNA spacer with the strand to which it the spacer complementary.

In some embodiments, a targeting moiety binds to an anchor sequence, e.g., an anchor sequence proximal to a target gene, e.g., MYC or associated with an anchor sequence-mediated conjunction (ASMC) operably linked to a target gene, e.g., MYC (e.g., an anchor sequence-mediated conjunction is operably linked to a target gene, e.g., MYC if disruption of the conjunction alters expression of the target gene, e.g., MYC) in mouse genome. In general, an anchor sequence is a genomic sequence element to which a genomic complex component, e.g., nucleating polypeptide binds specifically. In some embodiments, binding of a genomic complex component to an anchor sequence nucleates complex formation, e.g., ASMC formation. In some embodiments, a targeting moiety binds to a target gene, e.g., MYC locus. A locus is generally defined to encompass transcribed region, promoter, and anchor sites of an ASMC comprising a target gene, e.g., MYC. In some embodiments, a targeting moiety binds to a sequence comprising any one of SEQ ID NOS: 190-192. In some embodiments, the targeting moiety binds to a sequence comprising any one of the SEQ ID Nos. disclosed in Table 18. Exemplary target sequences in mouse genome are disclosed in Table 18.

TABLE 18
Exemplary target sequences in mouse genome

| | SEQ | | |
|---|---|---|---|
| | ID | | GENOMIC |
| NAME | NO: | SEQUENCE | COORDINATES |
| ZF15- | 190 | AACACAGTTCAGCCGAGCGCT | GRCm38: |
| MQ1 | | | chr15:61985053- |
| | | | 61985073 |
| ZF16- | 191 | CGAACAACCGTACAGAAAGGG | GRCm38: |
| MQ1 | | | chr15:61985079- |
| | | | 61985099 |
| ZF17- | 192 | GTAAACAGTAATAGCGCAGCA | GRCm38: |
| MQ1 | | | chr15:61985151- |
| | | | 61985171 |

In some embodiments, an expression repressor binds a genomic locus having a sequence set forth herein, e.g., any one of SEQ ID NOS: 190-192. It is understood that, in many cases, the genomic locus being bound comprises double stranded DNA, and this locus can be described by giving the sequence of its sense strand or its antisense strand.

In one embodiment, the anchor sequence-mediated conjunction comprises a loop, such as an intra-chromosomal loop. In certain embodiments, the anchor sequence-mediated conjunction has a plurality of loops. One or more loops may include a first anchor sequence, a nucleic acid sequence, a transcriptional control sequence, and a second anchor sequence. In another embodiment, at least one loop includes, in order, a first anchor sequence, a transcriptional control sequence, and a second anchor sequence, or a first anchor sequence, a nucleic acid sequence, and a second anchor sequence. In yet another embodiment, either one or both of the nucleic acid sequences and the transcriptional control sequence is located within or outside the loop. In still another embodiment, one or more of the loops comprises a transcriptional control sequence.

In some embodiments, the anchor sequence-mediated conjunction includes a TATA box, a CAAT box, a GC box, or a CAP site. In some embodiments, the anchor sequence-mediated conjunction comprises a plurality of loops, and where the anchor sequence-mediated conjunction comprises at least one of an anchor sequence, a nucleic acid sequence, and a transcriptional control sequence in one or more of the loops.

In some embodiments, chromatin structure is modified by substituting, adding, or deleting one or more nucleotides within an anchor sequence. In some embodiments, chromatin structure is modified by substituting, adding, or deleting one or more nucleotides within an anchor sequence of an anchor sequence-mediated conjunction. In some embodiments, transcription is inhibited by inclusion of an activating loop or exclusion of a repressive loop. In one such embodiment, the anchor sequence-mediated conjunction excludes a transcriptional control sequence that decreases transcription of the nucleic acid sequence. In some embodiments, transcription is repressed by inclusion of a repressive loop or exclusion of an activating loop. In one such embodiment, the anchor sequence-mediated conjunction includes a transcriptional control sequence that decreases transcription of the nucleic acid sequence.

The anchor sequences may be non-contiguous with one another. In embodiments with noncontiguous anchor sequences, the first anchor sequence may be separated from the second anchor sequence by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, the first anchor sequence is separated from the second anchor sequence by about 500 bp, 600 bp, 700 bp, 800 bp, 900 bp, 1 kb, 5 kb, 10 kb, 15 kb, kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 20 100 Mb, 200 Mb, 300 Mb, 400 Mb, 500 Mb, or any size therebetween.

In some more embodiments, the targeting moiety introduces at least one of the following: at least one exogenous anchor sequence; an alteration in at least one conjunction nucleating molecule binding site, such as by altering binding affinity for the conjunction nucleating molecule; a change in an orientation of at least one common nucleotide sequence, such as a CTCF binding motif, YY1 binding motif, ZNF143 binding motif, or other binding motif mentioned herein; and a substitution, addition or deletion in at least one anchor sequence, such as a CTCF binding motif, YY1 binding motif, ZNF143 binding motif, or other binding motif mentioned herein.

In some embodiments, an anchor sequence comprises a nucleating polypeptide binding motif, e.g., a CTCF-binding motif: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO: 71), where N is any nucleotide.

A CTCF-binding motif may also be in an opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO: 72). Where N is any nucleotide

In some embodiments, an anchor sequence comprises SEQ ID NO: 71 or SEQ ID NO: 72 or a sequence at least 75%, at least 80%, at least 90%, at least 95%, at least 96%, at least 97%, at least 98%, at least 99% identical to either SEQ ID NO: 71 or SEQ ID NO: 72.

In some embodiments, an anchor sequence comprises a nucleating polypeptide binding motif, e.g., a YY1-binding motif: CCGCCATNTT (SEQ ID NO: 73), where N is any nucleotide.

A YY1-binding motif may also be in an opposite orientation, e.g., AANATGGCGG (SEQ ID NO: 74), where N is any nucleotide.

In some embodiments, an anchor sequence-mediated conjunction comprises at least a first anchor sequence and a second anchor sequence. For example, in some embodiments, a first anchor sequence and a second anchor sequence may each comprise a nucleating polypeptide binding motif, e.g., each comprises a CTCF binding motif.

In some embodiments, a first anchor sequence and second anchor sequence comprise different sequences, e.g., a first anchor sequence comprises a CTCF binding motif, and a second anchor sequence comprises an anchor sequence other than a CTCF binding motif. In some embodiments, each anchor sequence comprises a nucleating polypeptide binding motif and one or more flanking nucleotides on one or both sides of a nucleating polypeptide binding motif.

Two CTCF-binding motifs (e.g., contiguous or non-contiguous CTCF binding motifs) that can form an ASMC may be present in a genome in any orientation, e.g., in the same orientation (tandem) either 5′-3′ (left tandem, e.g., the two CTCF-binding motifs that comprise SEQ ID NO:71) or 3′-5′ (right tandem, e.g., the two CTCF-binding motifs comprise SEQ ID NO:72), or convergent orientation, where one CTCF-binding motif comprises SEQ ID NO:71 and another other comprises SEQ ID NO:72.

In some embodiments, an anchor sequence comprises a CTCF binding motif associated with a target gene (e.g., MYC), wherein the target gene is associated with a disease, disorder and/or condition, e.g., MYC mis-regulating disorder, e.g., hepatic disorder, (e.g., hepatocarcinoma) or lung cancer.

In some embodiments, methods of the present disclosure comprise modulating, e.g., disrupting, a genomic complex (e.g., ASMC), e.g., by modifying chromatin structure, by substituting, adding, or deleting one or more nucleotides within an anchor sequence, e.g., a nucleating polypeptide binding motif. One or more nucleotides may be specifically targeted, e.g., a targeted alteration, for substitution, addition or deletion within an anchor sequence, e.g., a nucleating polypeptide binding motif.

In some embodiments, a genomic complex (e.g., ASMC) may be altered by changing an orientation of at least one nucleating polypeptide binding motif. In some embodiments, an anchor sequence comprises a nucleating polypeptide binding motif, e.g., CTCF binding motif, and a targeting moiety introduces an alteration in at least one nucleating polypeptide binding motif, e.g., altering binding affinity for a nucleating polypeptide.

In some embodiments, before administration of an expression repressor or system described herein, the target gene, e.g., MYC has a defined state of expression, e.g., in a diseased state. For example, the target gene, e.g., MYC may have a high level of expression in a disease cell. By disrupting the anchor sequence-mediated conjunction, expression of the target gene, e.g., MYC may be decreased.

A targeting moiety suitable for use in an expression repressor of an expression repression system described herein may bind, e.g., specifically bind, to a site comprising at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs (and optionally no more 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 nucleotides or base pairs). In some embodiments, a DNA-targeting moiety binds to a site comprising 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs.

Expression repression systems of the present disclosure may comprise two or more expression repressors. In some embodiments, the expression repressors of an expression repressor system each comprise a different targeting moiety.

In some embodiments, an expression repression system comprises a first expression repressor comprising a targeting moiety that binds a target gene, e.g., an exon, intron, or exon intron boundary (e.g., splice site), and second expression repressor comprising a targeting moiety that binds the target gene, e.g., an exon, intron, or exon intron boundary (e.g., splice site). In some embodiments, an expression repression system comprises a first expression repressor comprising a targeting moiety that binds a target gene, e.g., an exon, intron, or exon intron boundary (e.g., splice site), and second expression repressor comprising a targeting moiety that binds to a transcription control element (e.g., promoter or enhancer) operably linked to the target gene, e.g., MYC. In some embodiments, an expression repression system comprises a first expression repressor comprising a targeting moiety that binds to a transcription control element (e.g., promoter or enhancer) operably linked to a target gene, and a second expression repressor comprising a targeting moiety that binds to a transcription control element (e.g., promoter or enhancer) operably linked to the target gene. In some embodiments, an expression repression system comprises a first expression repressor comprising a targeting moiety that binds to an anchor sequence proximal to a target gene, e.g., MYC or associated with an anchor sequence-mediated conjunction operably linked to a target gene, e.g., MYC, and a second expression repressor comprising a targeting moiety that binds to a transcription control element (e.g., promoter or enhancer) operably linked to the target gene, e.g., MYC. In some embodiments, an expression repression system comprises a first expression repressor comprising a targeting moiety that binds to an anchor sequence proximal to a target gene, e.g., MYC or associated with an anchor sequence-mediated conjunction operably linked to a target gene, e.g., MYC, and a second expression repressor comprising a targeting moiety that binds to the target gene (e.g., MYC), e.g., an exon, intron, or exon intron boundary (e.g., splice site). In some embodiments, an expression repression system comprises a first expression repressor comprising a targeting moiety that binds to an anchor sequence proximal to a target gene, e.g., MYC or associated with an anchor sequence-mediated conjunction operably linked to a target gene, e.g., MYC, and a second expression repressor comprising a targeting moiety that binds to an anchor sequence proximal to the target gene, e.g., MYC or associated with an anchor sequence-mediated conjunction operably linked to the target gene, e.g., MYC.

In some embodiments, an expression repression system comprises a first expression repressor comprising a targeting moiety that binds to a first site, e.g., in a promoter operably linked to a target gene, e.g., MYC, and a second expression repressor comprising a targeting moiety that binds to a second site, e.g., in the promoter operably linked to a target gene, e.g., MYC. The first site and second site may be different and non-overlapping sites, e.g., the first site and second site do not share any sequence in common. The first site and second site may be different but overlapping sites, e.g., the first site and second site comprise different sequences but share some sequence in common.

In some embodiments, the target gene is MYC. In some embodiments, MYC is located on human chromosome 8. In some embodiments, the expressor repressor or the expression repressor system as described herein binds to the transcription start site (TSS) of MYC.

### Other Compositions

Nucleic Acids and Vectors

The present disclosure is further directed, in part, to nucleic acids encoding expression repressors or expression repression systems described herein. In some embodiments, an expression repressor may be provided via a composition comprising a nucleic acid encoding the expression repressor, wherein the nucleic acid is associated with sufficient other sequences to achieve expression of the expression repressor, in a system of interest (e.g., in a particular cell, tissue, organism, etc.). Iri some embodiments, an expression repression system may be provided via a composition comprising a nucleic acid encoding the expression repression system, e.g., expression repressor(s) of the expression repression system, wherein the nucleic acid is associated with sufficient other sequences to achieve expression of the expression repression system, e.g., expression repressor(s) of the expression repression system, in a system of interest (e.g., in a particular cell, tissue, organism, etc.).

In some particular embodiments, the present disclosure provides compositions of nucleic acids that encode an expression repressor or polypeptide portion thereof. In some such embodiments, provided nucleic acids may be or include DNA, RNA, or any other nucleic acid moiety or entity as described herein, and may be prepared by any technology described herein or otherwise available in the art (e.g., synthesis, cloning, amplification, in vitro or in vivo transcription, etc.). In some embodiments, provided nucleic acids that encode an expression repressor or polypeptide portion thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc.).

In some embodiments, a composition for delivering an expression repressor described herein is or comprises a vector, e.g., a viral vector, comprising one or more nucleic acids encoding an expression repressor or one or more components of an expression repressor as described herein.

In some particular embodiments, the present disclosure provides compositions of nucleic acids that encode an expression repression system, one or more expression repressors, or polypeptide portions thereof. In some such embodiments, provided nucleic acids may be or include DNA, RNA, or any other nucleic acid moiety or entity as described herein, and may be prepared by any technology described herein or otherwise available in the art (e.g., synthesis, cloning, amplification, in vitro or in vivo transcription, etc.). In some embodiments, provided nucleic acids that encode an expression repression system, one or more expression repressors, or polypeptide portions thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc.).

In some embodiments, a composition for delivering an expression repression system described herein is or comprises a vector, e.g., a viral vector, comprising one or more nucleic acids encoding one or more components of an expression repression system, e.g., expression repressor(s) of the expression repression system as described herein.

In some embodiments, a composition for delivering an expression repressor described herein is or comprises RNA, e.g., mRNA, comprising one or more nucleic acids encoding an expression repressor or one or more components of an expression repressor, as described herein.

In some embodiments, a composition for delivering an expression repression system described herein is or comprises RNA, e.g., mRNA, comprising one or more nucleic acids encoding one or more components of an expression repression system, e.g., expression repressor(s) of the expression repression system as described herein.

Nucleic acids as described herein or nucleic acids encoding a protein described herein, may be incorporated into a vector. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art, and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter and incorporating the construct into an expression vector. Vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well.

In some embodiments, an expression repressor or system described herein acts at an enhancing sequence. In some embodiments, the enhancing sequence is an enhancer, a stretch enhancer, a shadow enhancer, a locus control region (LCR), or a super enhancer. In some embodiments, the super enhancer comprises a cluster of enhancers and other regulatory elements. In some embodiments, these sequences are located in a region 0.2-2 Mb upstream or downstream of a transcription start site. In some embodiments, the region is a non-coding region. In some embodiments, the region contains at least one SNP associated with higher risk of developing cancer. In some embodiments, the region is associated with long-range regulation of a target gene, e.g., MYC. In some embodiments, the regions are cell-type specific. In some embodiments, a super-enhancer modifies (e.g., increases or decreases) target gene expression, e.g., MYC expression, by recruiting the target gene promoter, e.g., MYC promoter. In some embodiments, the super enhancer interacts with a target gene promoter, e.g., MYC promoter, through an enhancer docking site. In some embodiments, the enhancer docking site is an anchor sequence. In some embodiments, the enhancer docking site is located at least 100 bp, 200 bp, 500 bp, 1000 bp, 1500 bp, 2000 bp, or 3000 bp away from the target gene promoter, e.g., MYC promoter. In some embodiments, a super enhancer region is at least 100 bp, at least 200 bp, at least 300 bp, at least 500 bp, at least 1 kb, at least 2 kb, at least 3 kb, at least 5 kb, at least 10 kb, at least 15 kb, at least 20 kb, or at least 25 kb long.

Spacing between promoter elements frequently is flexible, so that promoter function is preserved when elements are inverted or moved relative to one another. In a thymidine kinase (tk) promoter, spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In some embodiments of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, an actin promoter, a myosin promoter, a hemoglobin promoter, and a creatine kinase promoter.

The present disclosure should not be interpreted to be limited to use of any particular promoter or category of promoters (e.g., constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate transcriptional control sequences to enable expression in the host cells. Useful selectable markers may include, for example, antibiotic-resistance genes, such as neo, etc.

In some embodiments, reporter genes may be used for identifying potentially transfected cells and/or for evaluating the functionality of transcriptional control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to modulate promoter-driven transcription.

Cells

The present disclosure is further directed, in part, to cells comprising an expression repressor or an expression repression system described herein. Any cell, e.g., cell line, e.g., a cell line suitable for expression of a recombinant polypeptide, known to one of skill in the art is suitable to comprise an expression repressor or an expression repression system described herein. In some embodiments, a cell, e.g., cell line, may be used to express an expression repressor or an expression repression system, e.g., expression repressor(s), described herein. In some embodiments, a cell, e.g., cell line, may be used to express or amplify a nucleic acid, e.g., a vector, encoding an expression repressor or an expression repression system, e.g., expression repressor(s), described herein. In some embodiments, a cell comprises a nucleic acid encoding an expression repressor or an expression repression system, e.g., expression repressor(s), described herein.

In some embodiments, a cell comprises a first nucleic acid encoding a first component of an expression repression system, e.g., a first expression repressor, and a second nucleic acid encoding a second component of the expression repression system, e.g., a second expression repressor. In some embodiments, wherein a cell comprises nucleic acid encoding an expression repression system comprising two or more expression repressors, the sequences encoding each expression repressor are disposed on separate nucleic acid molecules, e.g., on different vectors, e.g., a first vector encoding a first expression repressor and a second vector encoding a second expression repressor. In some embodiments, the sequences encoding each expression repressor are disposed on the same nucleic acid molecule, e.g., on the same vector. In some embodiments, some or all of the nucleic acid encoding the expression repression system is integrated into the genomic DNA of the cell. In some embodiments, the nucleic acid encoding a first expression repressor of an expression repression system is integrated into the genomic DNA of a cell, and the nucleic acid encoding a second expression repressor of an expression repression system is not integrated into the genomic DNA of a cell (e.g., is situated on a vector). In some embodiments, the nucleic acid(s) encoding a first and a second expression repressor of an expression repression system are integrated into the genomic DNA of a cell, e.g., at the same (e.g., adjacent or colocalized) or different sites in the genomic DNA.

Examples of cells that may comprise and/or express an expression repression system or expression repressor described herein include, but are not limited to, hepatocytes, neuronal cells, endothelial cells, myocytes, and lymphocytes.

The present disclosure is further directed, in part, to a cell made by a method or process described herein. In some embodiments, the disclosure provides a cell produced by: providing an expression repressor or an expression repression system described herein, providing the cell, and contacting the cell with the expression repressor (or a nucleic acid encoding the expression repressor, or a composition comprising said expression repressor or nucleic acid) or the expression repression system (or a nucleic acid encoding the expression repression system, or a composition comprising said expression repression system or nucleic acid). In some embodiments, contacting a cell with an expression repressor comprises contacting the cell with a nucleic acid encoding the expression repressor under conditions that allow the cell to produce the expression repressor. In some embodiments, contacting a cell with an expression repressor comprises contacting an organism that comprises the cell with the expression repressor or a nucleic acid encoding the expression repressor under conditions that allow the cell to produce the expression repressor.

Without wishing to be bound by theory, a cell contacted with an expression repressor or an expression repression system described herein may exhibit: a decrease in expression of a target gene (e.g., MYC) and/or a modification of epigenetic markers associated with the target gene, e.g., MYC, a transcription control element operably linked to the target gene, e.g., MYC, or an anchor sequence proximal to the target gene or associated with an anchor sequence-mediated conjunction operably linked to the target gene, e.g., MYC compared to a similar cell that has not been contacted by the expression repressor or the expression repression system. In some embodiments, a cell exhibiting said decrease in expression of a target gene, e.g., MYC and/or modification of epigenetic markers does not comprise the expression repressor or the expression repression system. The decrease in expression and/or modification of epigenetic markers may persist, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after contact with the expression repressor or the expression repression system.

In some embodiments, a cell previously contacted by an the expression repressor or an expression repression system retains the decrease in expression and/or modification of epigenetic markers after the expression repressor or the expression repression system is no longer present in the cell, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after the expression repressor or the expression repression system is no longer present in the cell.

Methods of Making Expression Repression Systems and/or Expression Repressors

In some embodiments, an expression repressor comprises or is a protein and may thus be produced by methods of making proteins. In some embodiments, an expression repression system, e.g., the expression repressor(s) of an expression repression system, comprise one or more proteins and may thus be produced by methods of making proteins. As will be appreciated by one of skill, methods of making proteins or polypeptides (which may be included in modulating agents as described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

A protein or polypeptide of compositions of the present disclosure can be biochemically synthesized by employing standard solid phase techniques. Such methods include exclusive solid phase synthesis, partial solid phase synthesis methods, fragment condensation, classical solution synthesis. These methods can be used when a peptide is relatively short (e.g., 10 kDa) and/or when it cannot be produced by recombinant techniques (i.e., not encoded by a nucleic acid sequence) and therefore involves different chemistry.

Solid phase synthesis procedures are well known in the art and further described by John Morrow Stewart and Janis Dillaha Young, Solid Phase Peptide Syntheses, 2^{nd} Ed., Pierce Chemical Company, 1984; and Coin, I., et al., Nature Protocols, 2:3247-3256, 2007.

For longer peptides, recombinant methods may be used. Methods of making a recombinant therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Exemplary methods for producing a therapeutic pharmaceutical protein or polypeptide involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the protein or polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein. Compositions described herein may include a lipid nanoparticle encapsulating a vector, such as a viral vector, e.g., a lentiviral vector, encoding a recombinant protein. In some embodiments, a lipid nanoparticle encapsulating a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

Proteins comprise one or more amino acids. Amino acids include any compound and/or substance that can be incorporated into a polypeptide chain, e.g., through formation of one or more peptide bonds. In some embodiments, an amino acid has the general structure H₂N—C(H)I—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

### Pharmaceutical Compositions, Formulation, Delivery, and Administration

The present disclosure is further directed, in part, to pharmaceutical compositions comprising an expression repressor or an expression repression system, e.g., expression repressor(s), described herein, to pharmaceutical compositions comprising nucleic acids encoding the expression repressor or the expression repression system, e.g., expression repressor(s), described herein, and/or to and/or compositions that deliver an expression repressor or an expression repression system, e.g., expression repressor(s), described herein to a cell, tissue, organ, and/or subject.

As used herein, the term “pharmaceutical composition” refers to an active agent (e.g., an expression repressor or nucleic acids of the expression receptor, e.g., an expression repression system, e.g., expression repressor(s) of an expression repressor system, or nucleic acid encoding the same), formulated together with one or more pharmaceutically acceptable carriers (e.g., pharmaceutically acceptable carriers known to those of skill in the art). In some embodiments, active agent is present in unit dose amount appropriate for administration in a therapeutic regimen that shows a statistically significant probability of achieving a predetermined therapeutic effect when administered to a relevant population. In some embodiments, a pharmaceutical composition comprising an expression repressor of the present disclosure comprises an expression repressor or nucleic acid(s) encoding the same. In some embodiments, a pharmaceutical composition comprising an expression repression system of the present disclosure comprises or each of the expression repressors of the expression repression system or nucleic acid(s) encoding the same (e.g., if an expression repression system comprises a first expression repressor and a second expression repressor, the pharmaceutical composition comprises the first and second expression repressor). In some embodiments, a pharmaceutical composition comprises less than all of the expression repressors of an expression repression system comprising a plurality of expression repressors. For example, an expression repression system may comprise a first expression repressor and a second expression repressor, and a first pharmaceutical composition may comprise the first expression repressor or nucleic acid encoding the same and a second pharmaceutical composition may comprise the second expression repressor or nucleic acid encoding the same. In some embodiments, a pharmaceutical composition may comprise coformulation of one or more expression repressors, or nucleic acid(s) encoding the same.

In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; trans-dermally; or nasally, pulmonary, and/or to other mucosal surfaces.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In some embodiments, for example, materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt”, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate, and aryl sulfonate.

In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical preparations may be made following conventional techniques of pharmacy involving milling, mixing, granulation, and compressing, when necessary, for tablet forms; or milling, mixing and filling for hard gelatin capsule forms. When a liquid carrier is used, a preparation can be in the form of a syrup, elixir, emulsion or an aqueous or non-aqueous solution or suspension. Such a liquid formulation may be administered directly per os.

In some embodiments, pharmaceutical compositions may be formulated for delivery to a cell and/or to a subject via any route of administration. Modes of administration to a subject may include injection, infusion, inhalation, intranasal, intraocular, topical delivery, inter-cannular delivery, or ingestion. Injection includes, without limitation, intravenous, intramuscular, intra-arterial, intrathecal, intraventricular, intracapsular, intra-orbital, intracardiac, intradermal, intraperitoneal, transtracheal, subcutaneous, subcuticular, intraarticular, sub capsular, subarachnoid, intraspinal, intra-cerebrospinal, and intra-sternal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, intravitreal injection). In some embodiments, one or more compositions is administered systemically. In some embodiments, administration is non-parenteral and a therapeutic is a parenteral therapeutic. In some embodiments, administration may be bronchial (e.g., by bronchial instillation), buccal, dermal (which may be or comprise, for example, one or more of topical to the dermis, intradermal, inter-dermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc.

In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, six, eight, ten, 12, 15 or 20 or more administrations may be given to the subject during one treatment or over a period of time as a treatment regimen.

In some embodiments, administrations may be given as needed, e.g., for as long as symptoms associated with the disease, disorder or condition persist. In some embodiments, repeated administrations may be indicated for the remainder of the subject's life. Treatment periods may vary and could be, e.g., one day, two days, three days, one week, two weeks, one month, two months, three months, six months, a year, or longer.

### Dosage

The dosage of the administered agent or composition can vary based on, e.g., the condition being treated, the severity of the disease, the subject's individual parameters, including age, physiological condition, size and weight, duration of treatment, the type of treatment to be performed (if any), the particular route of administration and similar factors. Thus, the dose administered of the agents described herein can depend on such various parameters. The dosage of an administered composition may also vary depending upon other factors as the subject's sex, general medical condition, and severity of the disorder to be treated. It may be desirable to provide the subject with a dosage of a modulatory agent or combination of modulatory agents disclosed herein that is in the range of from about 1 mg/kg to 6 mg/kg as a single intravenous infusion, although a lower or higher dosage also may be administered as circumstances dictate. The dosage may be repeated as needed, for example, once every day (e.g., for 1-30 days), once every 3 days (e.g., for 1-30 days) once every 5 days (e.g., for 1-30 days), once per week (e.g., for 1-6 weeks or for 2-5 weeks). In some embodiments, dosages may include, but are not limited to, 1.0 mg/kg-6 mg/kg, 1.0 mg/kg-5 mg/kg, 1.0 mg/kg-4 mg/kg, 1.0-3.0 mg/kg, 1.5 mg/kg-3.0 mg/kg, 1.0 mg/kg-1.5 mg/kg, 1.5 mg/kg-3 mg/kg, 3 mg/kg-4 mg/kg, 4 mg/kg-5 mg/kg, or 5 mg/kg-6 mg/kg. The dosage may be administered multiple times, e.g., once, or twice a week, or once every 1 or 2 weeks. In some embodiments, the subject is provided with a dosage of a modulatory agent or combination of modulatory agents disclosed herein that is in the range of from about 1 mg/kg to 6 mg/kg as multiple intravenous infusions although a lower or higher dosage also may be administered as circumstances dictate.

A modulatory agent or a combination of modulatory agents as disclosed herein may be administered as one dosage every 3-5 days, repeated for a total of at least 3 dosages. Alternatively, a modulatory agent or a combination of modulatory agents as disclosed herein may be administered at 3 mg/kg every 5 days for 25 days. Alternatively, a modulatory agent or a combination of modulatory agents as disclosed herein may be administered at 1.0-5.0 mg/kg every 3-5 days for 1-10 doses. Alternatively, a modulatory agent or a combination of modulatory agents as disclosed herein may be administered at 1.0-3.0 mg/kg every 5 days for 3 doses then every 3 days for 3 doses. Alternatively, a modulatory agent or a combination of modulatory agents as disclosed herein may be administered at 1.0-3.0 mg/kg every 5 days for 4 doses then every 3 days for 3 doses. Alternatively, a modulatory agent or a combination of modulatory agents as disclosed herein may be administered at 6 mg/kg every 5 days for 1-10 doses. Alternatively, a modulatory agent or a combination of modulatory agents as disclosed herein may be administered at 3 mg/kg every 5 days for 1-10 doses. Alternatively, a modulatory agent or a combination of modulatory agents as disclosed herein may be administered at 1.5 mg/kg every 5 days for 2 doses, 3 mg/kg every 5 days for 3 doses, 3 mg/kg every 3 days for 1 dose. Alternatively, a modulatory agent or a combination of modulatory agents as disclosed herein may be administered at 6 mg/kg at every 5 days or at 1.5 mg/kg once a day for 5 days with 2 days off. The dosing schedule can optionally be repeated at other intervals and dosage may be given through various parenteral routes, with appropriate adjustment of the dose and schedule. In some embodiments, the dosing of modulatory agents or a combination of modulatory agents may include a dosage of between 1.0 mg/kg to 6.0 mg/kg, optionally given either weekly, twice per week, or every other week. The person of ordinary skill will realize that a variety of factors, such as age, sex, weight, severity of disorder to be treated may be considered in selecting a dosage of a modulatory agent or a combination of modulatory agents as disclosed herein, and that the dosage and/or frequency of administration may be increased or decreased during the course of therapy. The dosage may be repeated as needed, with evidence of reduction of tumor volume observed after as few as 2 to 8 doses. The dosages and schedules of administration disclosed herein show minimal effect on overall weight of the subject compared to cisplatin, sorafenib, or a small molecule comparator. The subject methods may include use of CT and/or PET/CT, or MRI, to measure tumor response at regular intervals. Blood levels of tumor markers may also be monitored. Dosages and/or administration schedules may be adjusted as needed, according to the results of imaging and/or marker blood levels.

In some embodiments, the compositions disclosed herein may be administered in combination with one or more therapeutic agents or methods chosen from surgical resection, tyrosine kinase inhibitors (TKIs), e.g., sorafenib, bromodomain inhibitors, e.g., BET inhibitors, e.g., JQ1, e.g., BET672, e.g., birabresib, MEK inhibitors, (e.g., Trametinib), orthotopic liver transplantation, radiofrequency ablation, immunotherapy, immune checkpoint plus anti-vascular-endothelial-growth-factor combination therapy, photodynamic therapy (PDT), laser therapy, brachytherapy, radiation therapy, trans-catheter arterial chemo- or radio-embolization, stereotactic radiation therapy, chemotherapy, and/or systemic chemotherapy to treat a disease or disorder. Table 21 below discloses exemplary therapeutic agents.

TABLE 21
Small molecule compounds, e.g., for using in combination therapies
with expression repressors described herein.

| | |
|---|---|
| sorafenib | 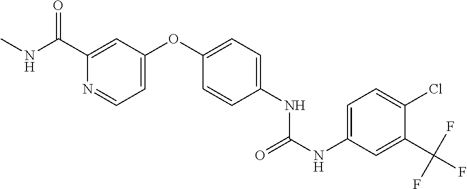 [SMILES: CNC(=O)c1cc(Oc2ccc(NC(=O)Nc3ccc(Cl)c(C(F)(F)F)c3)cc2)ccn1] |
| JQ1 | 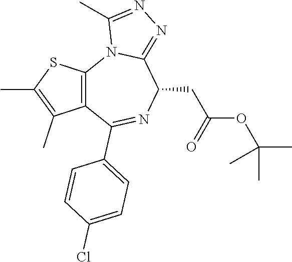 [SMILES: Cc1sc2c(c1C)C(c1ccc(Cl)cc1)=N[C@@H](CC(=O)OC(C)(C)C)c1nnc(C)n1-2] |
| BET762 | 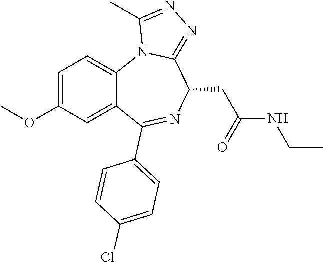 [SMILES: CCNC(=O)C[C@@H]1N=C(c2ccc(Cl)cc2)c2cc(OC)ccc2-n2c(C)nnc21] |
| birabresib | 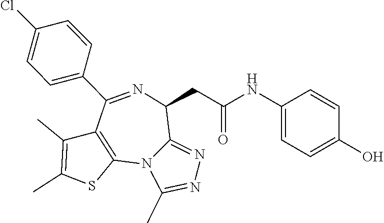 [SMILES: Cc1sc2c(c1C)C(c1ccc(Cl)cc1)=N[C@@H](CC(=O)Nc1ccc(O)cc1)c1nnc(C)n1-2] |
| trametinib | 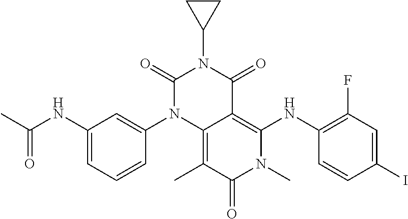 [SMILES: CC(=O)Nc1cccc(-n2c(=O)n(C3CC3)c(=O)c3c(Nc4ccc(I)cc4F)n(C)c(=O)c(C)c32)c1] |

Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

In some aspects, the present disclosure provides methods of delivering a therapeutic comprising administering a composition as described herein to a subject, wherein a modulating agent is a therapeutic and/or wherein delivery of a therapeutic causes changes in gene expression relative to gene expression in absence of a therapeutic.

Methods as provided in various embodiments herein may be utilized in any some aspects delineated herein. In some embodiments, one or more compositions is/are targeted to specific cells, or one or more specific tissues.

For example, in some embodiments one or more compositions is/are targeted to hepatic, epithelial, connective, muscular, reproductive, and/or nervous tissue or cells. In some embodiments a composition is targeted to a cell or tissue of a particular organ system, e.g., cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage); and/or combinations thereof.

In some embodiments, a composition of the present disclosure crosses a blood-brain-barrier, a placental membrane, or a blood-testis barrier.

In some embodiments, a pharmaceutical composition as provided herein is administered systemically.

In some embodiments, administration is non-parenteral and a therapeutic is a parenteral therapeutic.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

Pharmaceutical uses of the present disclosure may include compositions (e.g., modulating agents, e.g., disrupting agents) as described herein.

In some embodiments, a pharmaceutical composition of the present disclosure has improved PK/PD, e.g., increased pharmacokinetics or pharmacodynamics, such as improved targeting, absorption, or transport (e.g., at least 5%, 10%, 15%, 20%, 30%, 40%, 50%, 60%, 75%, 80%, 90% improved or more) as compared to an active agent alone. In some embodiments, a pharmaceutical composition has reduced undesirable effects, such as reduced diffusion to a nontarget location, off-target activity, or toxic metabolism, as compared to a therapeutic alone (e.g., at least 5%, 10%, 15%, 20%, 30%, 40%, 50%, 60%, 75%, 80%, 90% or more reduced, as compared to an active agent alone). In some embodiments, a composition increases efficacy and/or decreases toxicity of a therapeutic (e.g., at least 5%, 10%, 15%, 20%, 30%, 40%, 50%, 60%, 75%, 80%, 90% or more) as compared to an active agent alone.

Pharmaceutical compositions described herein may be formulated for example including a carrier, such as a pharmaceutical carrier and/or a polymeric carrier, e.g., a nanoparticle, a liposome or vesicle, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. July 2015, 26(7): 443-451. Doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

### Lipid Nanoparticles

Expression repressors or expression repression systems as described herein can be delivered using any biological delivery system/formulation including a particle, for example, a nanoparticle delivery system. Nanoparticles include particles with a dimension (e.g. diameter) between about 1 and about 1000 nanometers, between about 1 and about 500 nanometers in size, between about 1 and about 100 nm, between about 30 nm and about 200 nm, between about 50 nm and about 300 nm, between about 75 nm and about 200 nm, between about 100 nm and about 200 nm, and any range therebetween. A nanoparticle has a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In some embodiments, nanoparticles have a greatest dimension ranging between 25 nm and 200 nm. Nanoparticles as described herein comprise delivery systems that may be provided in any form, including but not limited to solid, semi-solid, emulsion, or colloidal nanoparticles. A nanoparticle delivery system may include but not limited to lipid-based systems, liposomes, micelles, micro-vesicles, exosomes, or gene gun. In one embodiment, the nanoparticle is a lipid nanoparticle (LNP). In some embodiments, the LNP is a particle that comprises a plurality of lipid molecules physically associated with each other by intermolecular forces.

In some embodiments, an LNP may comprise multiple components, e.g., 3-4 components. In one embodiment, the expression repressor or a pharmaceutical composition comprising said expression repressor (or a nucleic acid encoding the same, or pharmaceutical composition comprising said expression repressor nucleic acid) is encapsulated in an LNP. In one embodiment, the expression repression system or a pharmaceutical composition comprising said expression repression system (or a nucleic acid encoding the same, or pharmaceutical composition comprising said expression repression system nucleic acid) is encapsulated in an LNP. In some embodiments, the nucleic acid encoding the first expression repressor and the nucleic acid encoding the second expression repressor are present in same LNP. In some embodiments, the nucleic acid encoding the first expression repressor and the nucleic acid encoding the second expression repressor are present in different LNPs. Preparation of LNPs and the modulating agent encapsulation may be used/and or adapted from Rosin et al, Molecular Therapy, vol. 19, no. 12, pages 1286-2200, December 2011). In some embodiments, lipid nanoparticle compositions disclosed herein are useful for expression of protein encoded by mRNA. In some embodiments, nucleic acids, when present in the lipid nanoparticles, are resistant in aqueous solution to degradation with a nuclease.

In some embodiments, the LNP formulations may include a CCD lipid, a neutral lipid, and/or a helper lipid. In some embodiments, the LNP formulation comprises an ionizable lipid. In some embodiments, an ionizable lipid may be a cationic lipid, an ionizable cationic lipid, or an amine-containing lipid that can be readily protonated. In some embodiments, the lipid is a cationic lipid that can exist in a positively charged or neutral form depending on pH. In some embodiments, the cationic lipid is a lipid capable of being positively charged, e.g., under physiological conditions. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol, and polymer conjugated lipids.

In some embodiments, LNP formulation (e.g., MC3 and/or SSOP) includes cholesterol, PEG, and/or a helper lipid. The LNPs may be, e.g., microspheres (including unilamellar and multilamellar vesicles, lamellar phase lipid bilayers that, in some embodiments, are substantially spherical.

In some embodiments, the LNP can comprise an aqueous core, e.g., comprising a nucleic acid encoding an expression repressor or a system as disclosed herein. In some embodiments of the present disclosure, the cargo for the LNP formulation includes at least one guide RNA. In some embodiments, the cargo, e.g., a nucleic acid encoding an expression repressor, or a system as disclosed herein, may be adsorbed to the surface of an LNP, e.g., an LNP comprising a cationic lipid. In some embodiments, the cargo, e.g., a nucleic acid encoding an expression repressor, or a system as disclosed herein may be associated with the LNP. In some embodiments, the cargo, e.g., a nucleic acid encoding an expression repressor, or a system as disclosed herein, may be encapsulated, e.g., fully encapsulated and/or partially encapsulated in an LNP.

In some embodiments, an LNP comprising a cargo may be administered for systemic delivery, e.g., delivery of a therapeutically effective dose of cargo that can result in a broad exposure of an active agent within an organism. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery. In some embodiments, an LNP comprising a cargo may be administered for local delivery, e.g., delivery of an active agent directly to a target site within an organism. In some embodiments, an LNP may be locally delivered into a disease site, e.g., a tumor, other target site, e.g., a site of inflammation, or to a target organ, e.g., the liver, lung, stomach, colon, pancreas, uterus, breast, lymph nodes, and the like. In some embodiments, an LNP as disclosed herein may be locally delivered to a specific cell, e.g., hepatocytes, stellate cells, Kupffer cells, endothelial, alveolar, and/or epithelial cells. In some embodiments, an LNP as disclosed herein may be locally delivered to a specific tumor site, e.g., subcutaneous, orthotopic.

The LNPs may be formulated as a dispersed phase in an emulsion, micelles, or an internal phase in a suspension. In some embodiments, the LNPs are biodegradable. In some embodiments, the LNPs do not accumulate to cytotoxic levels or cause toxicity in vivo at a therapeutically effective dose. In some embodiments, the LNPs do not accumulate to cytotoxic levels or cause toxicity in vivo after repeat administrations at a therapeutically effective dose. In some embodiments, the LNPs do not cause an innate immune response that leads to a substantially adverse effect at a therapeutically effective dose.

In some embodiments, the LNP used, comprises the formula (6Z,9Z,28Z,31Z)-heptatriacont-6,9,28,31-tetraene-19-yl 4-(dimethylamino)butanoate or ssPalmO-phenyl-P4C2 (ssPalmO-Phe, SS-OP). In some embodiments, the LNP formulation comprises the formula, (6Z,9Z,28Z,31Z)-heptatriacont-6,9,28,31-tetraene-19-yl 4-(dimethylarnino)butanoate (MC3), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., MC3 LNP or ssPalmO-phenyl-P4C2 (ssPalmO-Phe, SS-OP), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., SSOP-LNP.

Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. Doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. Doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

### Uses

The present disclosure is further directed to uses of the expression repressors or expression repressor systems disclosed herein. Among other things, in some embodiments such provided technologies may be used to achieve modulation, e.g., repression, of target gene, e.g., MYC expression and, for example, enable control of target gene, e.g., MYC activity, delivery, and penetrance, e.g., in a cell. In some embodiments, a cell is a mammalian, e.g., human, cell. In some embodiments, a cell is a somatic cell. In some embodiments, a cell is a primary cell. For example, in some embodiments, a cell is a mammalian somatic cell. In some embodiments, a mammalian somatic cell is a primary cell. In some embodiments, a mammalian somatic cell is a non-embryonic cell.

### Modulating Gene Expression

The present disclosure is further directed, in part, to a method of modulating, e.g., decreasing, expression of a target gene, e.g., MYC, comprising providing an expression repressor (or a nucleic acid encoding the same, or pharmaceutical composition comprising said expression repressor nucleic acid) or an expression repression system described herein (or a nucleic acid encoding the same, or pharmaceutical composition comprising said expression repression system or nucleic acid), and contacting the target gene e.g., MYC, and/or operably linked transcription control element(s) with the expression repressor or the expression repression system. In some embodiments, modulating, e.g., decreasing expression of a target gene, e.g., MYC comprises modulation of transcription of a target gene, e.g., MYC as compared with a reference value, e.g., transcription of a target gene, e.g., MYC in absence of the expression repressor or the expression repression system. In some embodiments, the method of modulating, e.g., decreasing, expression of a target gene, e.g., MYC are used ex vivo, e.g., on a cell from a subject, e.g., a mammalian subject, e.g., a human subject. In some embodiments, the method of modulating, e.g., decreasing, expression of a target gene, e.g., MYC are used in vivo, e.g., on a mammalian subject, e.g., a human subject. In some embodiments, the method of modulating, e.g., decreasing, expression of a target gene, e.g., MYC are used in vitro, e.g., on a cell or cell line described herein.

The present disclosure is further directed, in part to a method of treating a condition associated with mis-regulation, e.g., over-expression of a target gene, e.g., MYC in a subject, comprising administering to the subject an expression repressor (or a nucleic acid encoding the same, or pharmaceutical composition comprising said expression repressor nucleic acid) or an expression repression system described herein (or a nucleic acid encoding the same, or pharmaceutical composition comprising said expression repression system or nucleic acid). Conditions associated with over-expression of particular genes are known to those of skill in the art. Such conditions include, but are not limited to, metabolic disorders, cancer (e.g., solid tumors), and hepatitis.

Methods and compositions as provided herein may treat a condition associated with over-expression or mis-regulation of a target gene, e.g., MYC by stably or transiently altering (e.g., decreasing) transcription of a target gene, e.g., MYC. In some embodiments, such a modulation persists for at least about 1 hour to about 30 days, or at least about 2 hours, 6 hours, 12 hours, 18 hours, 24 hours, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., permanently or indefinitely). Optionally, such a modulation persists for no more than 10, 9, 8, 7, 6, 5, 4, 3, 2, or 1 years.

In some embodiments, a method or composition provided herein may decrease expression of a target gene, e.g., MYC in a cell by at least 10, 20, 30, 40, 50, 60, 70, 80, 90, or 100% (and optionally up to 100%) relative to expression of the target gene in a cell not contacted by the composition or treated with the method.

In some embodiments, a method provided herein may modulate, e.g., decrease, expression of a target gene, e.g., MYC by disrupting a genomic complex, e.g., an anchor sequence-mediated conjunction, associated with said target gene. A gene that is associated with an anchor sequence-mediated conjunction may be at least partially within a conjunction (that is, situated sequence-wise between a first and second anchor sequences), or it may be external to a conjunction in that it is not situated sequence-wise between a first and second anchor sequences, but is located on the same chromosome and in sufficient proximity to at least a first or a second anchor sequence such that its expression can be modulated by controlling the topology of the anchor sequence-mediated conjunction. Those of ordinary skill in the art will understand that distance in three-dimensional space between two elements (e.g., between the gene and the anchor sequence-mediated conjunction) may, in some embodiments, be more relevant than distance in terms of base pairs. In some embodiments, an external but associated gene is located within 2 Mb, within 1.9 Mb, within 1.8 Mb, within 1.7 Mb, within 1.6 Mb, within 1.5 Mb, within 1.4 Mb, with 1.3 Mb, within 1.3 Mb, within 1.2 Mb, within 1.1 Mb, within 1 Mb, within 900 kb, within 800 kb, within 700 kb, within 500 kb, within 400 kb, within 300 kb, within 200 kb, within 100 kb, within 50 kb, within 20 kb, within 10 kb, or within 5 kb of the first or second anchor sequence.

In some embodiments, modulating expression of a gene, e.g., MYC comprises altering accessibility of a transcriptional control sequence to a gene, e.g., MYC. A transcriptional control sequence, whether internal or external to an anchor sequence-mediated conjunction, can be an enhancing sequence or a silencing (or repressive) sequence.

In some embodiments, such provided technologies may be used to treat a gene mis-regulation disorder e.g., MYC gene mis-regulation disorder e.g., a symptom associated with a MYC gene mis-regulation in a subject, e.g., a patient, in need thereof. In some embodiments, such provided technologies may be used to treat a MYC gene mis-regulation disorder or a symptom associated with a MYC gene mis-regulation disorder in a subject, e.g., a patient, in need thereof. In some embodiments, the disorder is associated with MYC mis-regulation, e.g., MYC overexpression. In some embodiments, the disorder is associated with AFP mis-regulation, e.g., AFP overexpression. In some embodiments, such provided technologies may be used to methylate the promoter of a target gene, e.g., MYC, to treat a gene mis-regulation disorder e.g., MYC gene mis-regulation disorder, e.g., a symptom associated with a MYC gene mis-regulation in a subject, e.g., a patient, in need thereof. In some embodiments, such provided technologies may selectively affect the viability of a cell which aberrantly expresses a polypeptide encoded by a target gene, e.g., MYC.

In some embodiments, such provided technologies may be used to treat a hepatic disorder or a disorder e.g. a symptom associated with a hepatic disorder in a subject, e.g., a patient, in need thereof. In some embodiments, such provided technologies may be used to treat a pulmonary disorder or a disorder e.g. a symptom associated with a hepatic disorder in a subject, e.g., a patient, in need thereof. In some embodiments, such provided technologies may be used to treat a neoplasia disorder e.g. a disorder or, a symptom associated with a neoplasia disorder in a subject, e.g., a patient, in need thereof. In some embodiments, such provided technologies may be used to treat a viral infection related disorder e.g. a disorder or a symptom associated with viral infection related disorder in a subject, e.g., a patient, in need thereof. In some embodiments, such provided technologies may be used to treat an alcohol misuse related disorder e.g. a disorder or a symptom associated with an alcohol misuse related disorder in a subject, e.g., a patient, in need thereof. In some embodiments, such provided technologies may be used to treat a neoplasia disorder associated with a viral infection or alcohol misuse, e.g., a disorder or a symptom associated with a neoplasia disorder that is associated with a viral infection or alcohol misuse in a subject, e.g., a patient, in need thereof.

In some embodiments, the condition treated is neoplasia. In some embodiments, the condition treated is tumorigenesis. In some embodiments, the condition treated is cancer. In some embodiments, the cancer is associated with poor prognosis. In some embodiments, the cancer is associated with MYC mis-regulation, e.g., MYC overexpression. In some embodiments, the cancer is associated with AFP mis-regulation, e.g., AFP overexpression. In some embodiments, the cancer is a breast, a hepatic, a colorectal, a lung, a pancreatic, a gastric, and/or a uterine cancer. In some embodiments, the cancer is associated with an infection, e.g., viral, e.g., bacterial. In some embodiments, the cancer is associated with alcohol abuse. In some embodiments, the cancer is hepatocarcinoma.

In some embodiments, the cancer cells are lung cancer cells, gastric, gastrointestinal, colorectal, pancreatic or hepatic cancer cells. In some embodiments, the cancer is hepatocellular carcinoma (HCC), Fibrolamellar Hepatocellular Carcinoma (FHCC), cholangiocarcinoma, Angiosarcoma, secondary liver cancer, non-small cell lung cancer (NSCLC), adenocarcinoma, small cell lung cancer (SCLC), large cell (undifferentiated) carcinoma, triple negative breast cancer, gastric adenocarcinoma, endometrial carcinoma, or pancreatic carcinoma.

In some embodiments the condition treated is a hepatic disease. In some embodiments the condition treated is associated with MYC mis-regulation, e.g., MYC overexpression. In some embodiments the condition treated is a chronic disease. In some embodiments the condition treated is a chronic liver disease. In some embodiments the condition treated is a viral infection. In some embodiments, the condition treated is an alcohol misuse associated disorder.

In some embodiments the condition treated is a pulmonary disease. In some embodiments the condition treated is associated with MYC mis-regulation, e.g., MYC overexpression. In some embodiments the condition treated is a chronic disease. In some embodiments the condition treated is a chronic pulmonary disease. In some embodiments, such provided technologies may be used to treat or reduce lung cancer growth, metastasis, drug resistance, and/or cancer stem cell (CSC) maintenance. In some embodiments, the condition treated is a carcinoma, e.g., non-small cell lung cancer (NSCLC). In some embodiments, the chronic pulmonary disease is associated with tobacco misuse.

In some embodiments, the cancer hepatocarcinoma subtype S1 (HCC S1), hepatocarcinoma subtype S2 (HCC S2), or hepatocarcinoma subtype S3 (HCC S2). In some embodiments, the HCC subtype is associated with MYC overexpression. In some embodiments, the cancer is HCC S1 or HCC S2. In some embodiments, the cancer subtype is associated with aggressive tumor and poor clinical outcome.

In some embodiments, the disclosure provides a treatment regimen that may be devised for the subject on the basis of the HCC subtype in the subject, e.g., a personalized approach to tailor the aggressiveness of treatment based on HCC subtype on a subject. In some embodiments, the disclosure provides a method of treatment using the expression repressors or expression repressor systems disclosed herein, the method comprising, identifying the HCC subtype in a patient and determine a dosage and administration schedule of said expression repressors and/or expression repressor systems based on the HCC subtype identification.

Methods are described herein to deliver agents, or a composition as disclosed herein to a subject for treatment of a disorder such that the subject suffers minimal side effects or systemic toxicity in comparison to chemotherapy treatment. In some embodiments, the subject does not experience any significant side effects typically associated with chemotherapy, when treated with the agents and/or compositions described herein. In some embodiments, the subject does not experience a significant side effect including but not limited to alopecia, nausea, vomiting, poor appetite, soreness, neutropenia, anemia, thrombocytopenia, dizziness, fatigue, constipation, oral ulcers, itchy skin, peeling, nerve and muscle damage, auditory changes, weight loss, diarrhea, immunosuppression, bruising, heart damage, bleeding, liver damage, kidney damage, edema, mouth and throat sores, infertility, fibrosis, epilation, moist desquamation, mucosal dryness, vertigo and encephalopathy when treated with the agents and/or compositions described herein. In some embodiments, the subject does not show a significant loss of body weight when treated with the agents and/or compositions described herein.

The agents and compositions described herein can be administered to a subject, e.g., a mammal, e.g., in vivo, to treat or prevent a variety of disorders as described herein. This includes disorders involving cells characterized by altered expression patterns of MYC.

### Epigenetic Modification

The present disclosure is further directed, in part, to a method of epigenetically modifying a target gene, a transcription control element operably linked to a target gene, or an anchor sequence (e.g., an anchor sequence proximal to a target gene or associated with an anchor sequence-mediated conjunction operably linked to a target gene), the method comprising providing an expression repressor (or nucleic acid encoding the same) or an expression repression system (e.g., expression repressor(s)), or nucleic acid encoding the same or pharmaceutical composition comprising said an expression repressor (or nucleic acid encoding the same) or expression repression system or nucleic acid; and contacting the target gene or a transcription control element operably linked to the target gene with the expression repressor or the expression repression system, thereby epigenetically modifying the target gene, e.g., MYC or a transcription control element operably linked to the target gene, e.g., MYC.

In some embodiments, a method of epigenetically modifying a target gene, e.g., MYC or a transcription control element operably linked to a target gene, e.g., MYC comprises increasing or decreasing DNA methylation of the target gene, e.g., MYC or a transcription control element operably linked to a target gene, e.g., MYC. In some embodiments, a method of epigenetically modifying a target gene, e.g., MYC or a transcription control element operably linked to a target gene, e.g., MYC comprises increasing or decreasing histone methylation of a histone associated with the target gene, e.g., MYC or a transcription control element operably linked to a target gene, e.g., MYC. In some embodiments, a method of epigenetically modifying a target gene, e.g., MYC or a transcription control element operably linked to a target gene, e.g., MYC comprises decreasing histone acetylation of a histone associated with the target gene, e.g., MYC or a transcription control element operably linked to a target gene, e.g., MYC. In some embodiments, a method of epigenetically modifying a target gene, e.g., MYC or a transcription control element operably linked to a target gene, e.g., MYC comprises increasing or decreasing histone sumoylation of a histone associated with the target gene, e.g., MYC or a transcription control element operably linked to a target gene, e.g., MYC. In some embodiments, a method of epigenetically modifying a target gene, e.g., MYC or a transcription control element operably linked to a target gene, e.g., MYC comprises increasing or decreasing histone phosphorylation of a histone associated with the target gene, e.g., MYC or a transcription control element operably linked to a target gene, e.g., MYC.

In some embodiments, a method of epigenetically modifying a target gene, e.g., MYC or a transcription control element operably linked to a target gene, e.g., MYC may decrease the level of the epigenetic modification by at least 10, 20, 30, 40, 50, 60, 70, 80, 90, or 100% (and optionally up to 100%) relative to the level of the epigenetic modification at that site in a cell not contacted by the composition or treated with the method. In some embodiments, a method of epigenetically modifying a target gene, e.g., MYC or a transcription control element operably linked to a target gene, e.g., MYC may increase the level of the epigenetic modification by at least 10, 20, 30, 40, 50, 60, 70, 80, 90, 100, 120, 140, 160, 180, 200, 300, 400, 500, 600, 700, 800, 900, or 1000% (and optionally up to 200, 300, 400, 500, 600, 700, 800, 900, 1000, or 2000%) relative to the level of the epigenetic modification at that site in a cell not contacted by the composition or treated with the method. In some embodiments, epigenetic modification of a target gene, e.g., MYC or a transcription control element operably linked to a target gene, e.g., MYC may modify the level of expression of the target gene, e.g., MYC, e.g., as described herein.

In some embodiments, an epigenetic modification produced by a method described herein persists for at least about 1 hour to about 30 days, or at least about 2 hours, 6 hours, 12 hours, 18 hours, 24 hours, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, an expression repressor, or an expression repression system for use in a method of epigenetically modifying a target gene, e.g., MYC or a transcription control element operably linked to a target gene, e.g., MYC comprises an expression repressor comprising an effector moiety that is or comprises an epigenetic modifying moiety.

For example, a effector moiety may be or comprise an epigenetic modifying moiety with DNA methyltransferase activity, and an endogenous or naturally occurring target sequence (e.g. a target gene, e.g., MYC or transcription control element) may be altered to increase its methylation (e.g., decreasing interaction of a transcription factor with a portion of target gene, e.g., MYC or transcription control element, decreasing binding of a nucleating protein to an anchor sequence, and/or disrupting or preventing an anchor sequence-mediated conjunction), or may be altered to decrease its methylation (e.g., increasing interaction of a transcription factor with a portion of a target gene, e.g., MYC or transcription control element, increasing binding of a nucleating protein to an anchor sequence, and/or promoting or increasing strength of an anchor sequence-mediated conjunction).

### Kits

The present disclosure further directed, in part, to a kit comprising an expression repressor or an expression repression system, e.g., expression repressor(s), described herein. In some embodiments, a kit comprises an expression repressor or an expression repression system (e.g., the expression repressor(s) of the expression repression system) and instructions for the use of said an expression repressor or expression repression system. In some embodiments, a kit comprises a nucleic acid encoding the expression repressor or a nucleic acid encoding the expression repression system or a component thereof (e.g., the expression repressor(s) of the expression repression system) and instructions for the use of said expression repressor (and/or said nucleic acid) and/or said expression repression system (and/or said nucleic acid). In some embodiments, a kit comprises a cell comprising a nucleic acid encoding the expression repressor or a nucleic acid encoding the expression repression system or a component thereof (e.g., the expression repressor(s) of the expression repression system) and instructions for the use of said cell, nucleic acid, and/or said expression repressor or expression repression system.

In some aspects, the kit comprises a) a container comprising a composition comprising a system comprising two expression repressors, comprising a first expression repressor comprising a first targeting moiety and optionally a first effector moiety, wherein the first expression repressor binds to a transcription regulatory element (e.g., a promoter or transcription start site (TSS)) operably linked to target gene, e.g., MYC or to a sequence proximal to the transcription regulatory element and a expression repressor comprising a second targeting moiety and optionally a second effector moiety, wherein the second expression repressor binds to an anchor sequence of an anchor sequence mediated conjunction (ASMC) comprising target gene, e.g., MYC or to a sequence proximal to the anchor sequence.

In some aspects, the kit comprises a) a container comprising a composition comprising a system comprising two expression repressors, comprising a first expression repressor comprising a first targeting moiety and optionally a first effector moiety, wherein the first expression repressor binds to a transcription regulatory element (e.g., a promoter or transcription start site (TSS)) operably linked to target gene, e.g., MYC or to a sequence proximal to the transcription regulatory element and an expression repressor comprising a second targeting moiety and optionally a second effector moiety, wherein the second expression repressor binds to a genomic locus located in a super enhancer region of a target gene, e.g., MYC.

In some embodiments the kit further comprises h) a set of instructions comprising at least one method for treating a disease or modulating, e.g., decreasing the expression of target gene, e.g., MYC within a cell with said composition. In some embodiments, the kits can optionally include a delivery vehicle for said composition (e.g., a lipid nanoparticle). The reagents may be provided suspended in the excipient and/or delivery vehicle or may be provided as a separate component which can be later combined with the excipient and/or delivery vehicle. In some embodiments, the kits may optionally contain additional therapeutics to be co-administered with the compositions to affect the desired target gene expression, e.g., MYC gene expression modulation. While the instructional materials typically comprise written or printed materials, they are not limited to such. Any medium capable of storing such instructions and communicating them to an end user is contemplated. Such media include but are not limited to electronic storage media (e.g., magnetic discs, tapes, cartridges, chips), optical media (e.g., CD ROM), and the like. Such media may include addresses to internet sites that provide such instructional materials.

In some embodiments, a kit comprises a unit dosage of an expression repressor an expression repression system, e.g., expression repressor(s), described herein, or a unit dosage of a nucleic acid, e.g., a vector, encoding an expression repression system, e.g., expression repressor(s), described herein.

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### EXAMPLES

### Example 1: Targeted Modification of the CTCF Motif Results in Down-Regulation of MYC

This example describes nuclease editing of the CTCF motif or the region adjacent to CTCF motif with Cas9 to down-regulate MYC expression.

In this example, four sgRNA complementary to the promoter region CpG island contained in exon 1 were designed to identify the best target region to direct epigenetic effector mediated down-regulation of the MYC genes. The c-MYC gene contains a long, untranslated exon 1 (Spencer CA, Groudine M 1991), with exons 2 and 3 containing the major coding regions. Genomic editing was performed using the CRISPR-dCas9-KRAB and CRISPR-dCas9-MQ1 (DNMT from the bacteria Mollicutes spiroplasma) effectors targeted by four sgRNA to regions of the MYC promoter CpG island in order to identify suitable down-regulatory regions. Both (1) dCas9 (no effector) combined with sgRNA, and (2) untreated cells were used as control to assess changes to MYC expression. These initial screens to select sgRNA were performed in easily grown and transfectable cells K562 and HEK293 and determined that sgRNA, GD-28617, mediated the strongest effect on MYC mRNA down-regulation (data not shown).

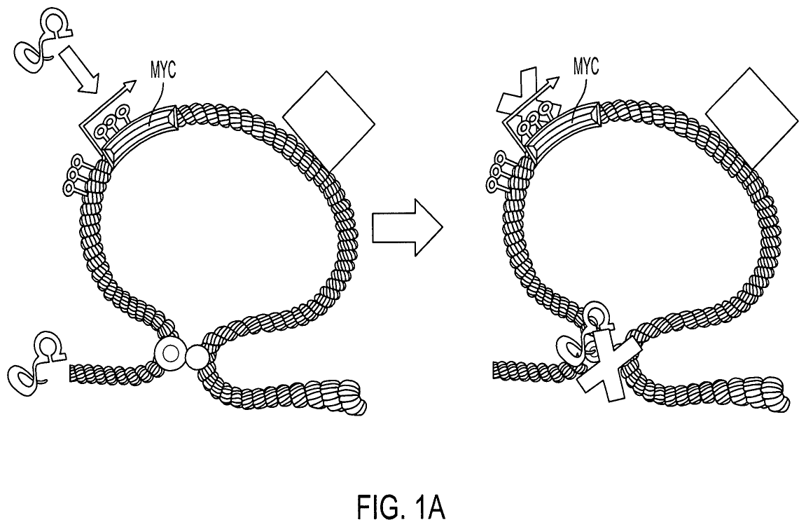

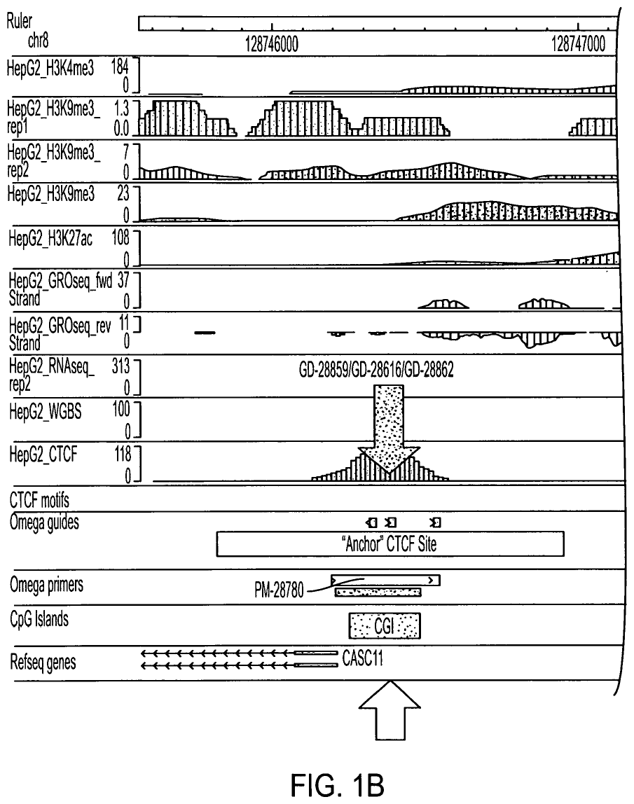

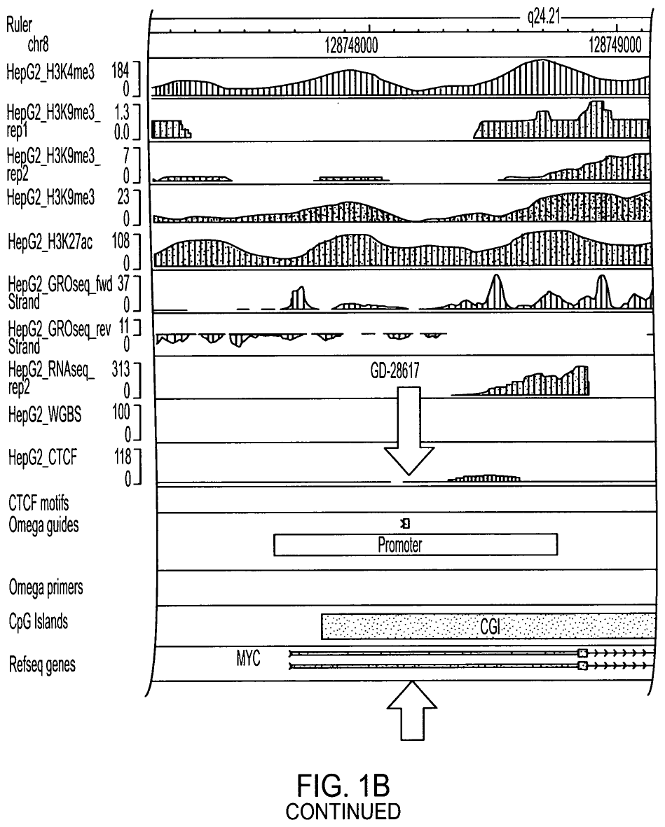

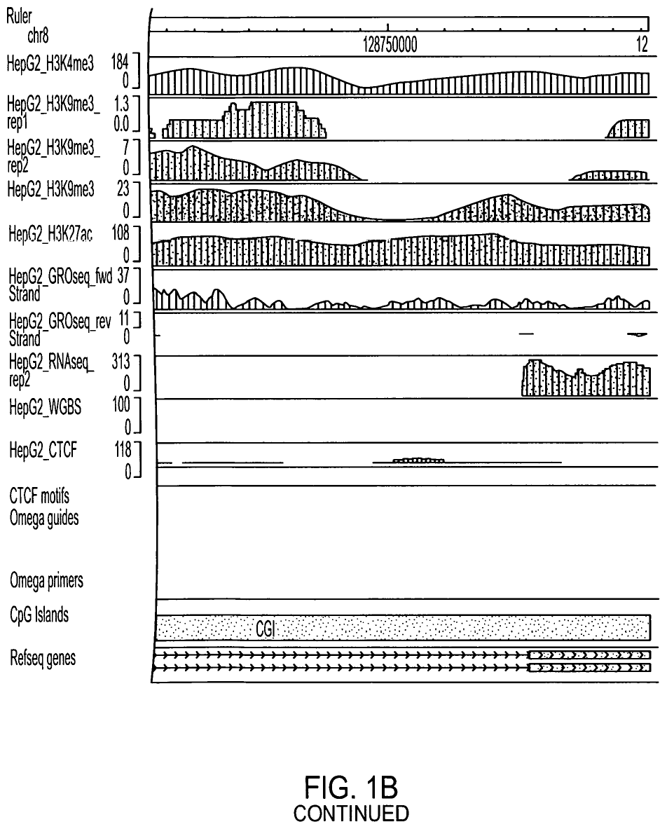

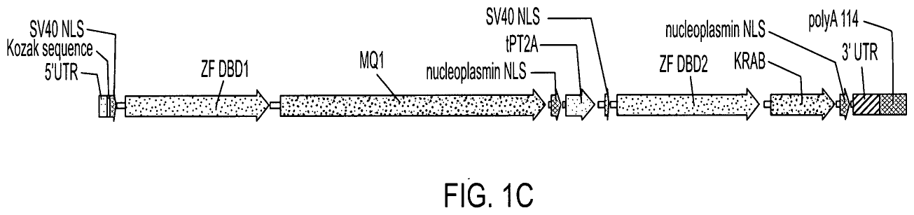

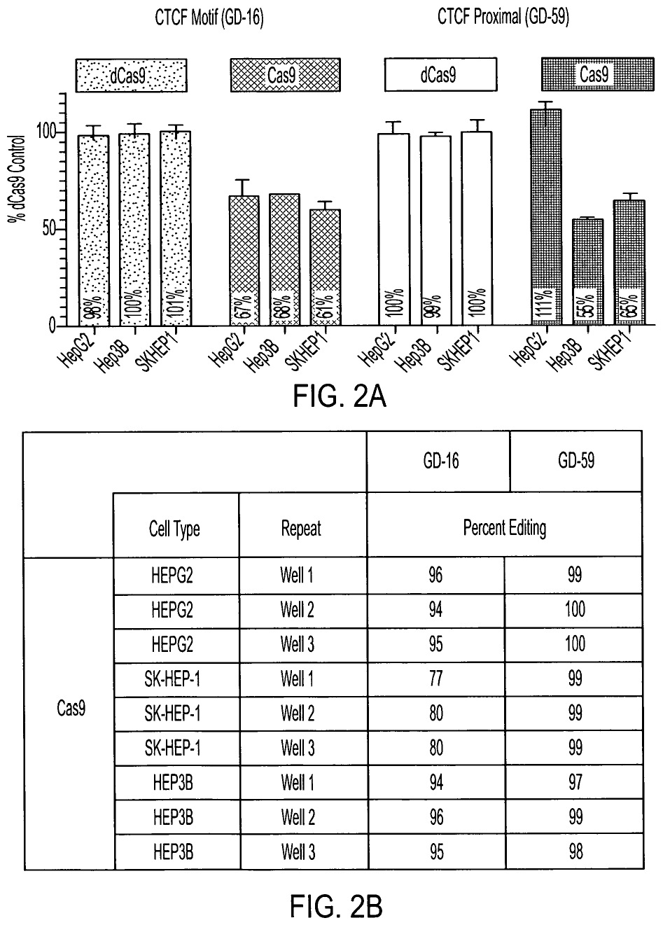

Cas9 targeted to the CTCF motif (GD-28616) or the upstream region directly adjacent to the CTCF motif (GD-28859) (a CTCF anchor site) was transfected into three human HCC models (HepG2, Hep3B and SKHEP1) with 2.5 ug/ml of SSOP formulation (Table 11 and FIG. 1A-B). Disruption of the CTCF motif with Cas9 (in combination with GD-28616) resulted in a 32-39% down-regulation in MYC expression in all three HCC cell lines (HepG2, Hep3B and SKHEP1). Disruption of the region adjacent to the CTCF motif (GD-28859) down-regulated MYC expression 35-45% in two (HepG2 and Hep3B) of the three lines (FIG. 2A). Editing efficiency as assessed by AmpSeq confirmed 77-100% editing of the cell lines (FIG. 2B).

TABLE 11
Guides

| Guide Name | Target Site | Target Sequence | Genomic Coordinates |
|---|---|---|---|
| GD-28616 | CTCF | ATGATCTCTGCTGCCAGTAG | chr8:128746342-128746364 |
| | | (SEQ ID NO: 1) | |
| GD-28859 | CTCF | ATCGCGCCTGGATGTCAACG | chr8:128746321-128746343 |
| | | (SEQ ID NO: 2) | |
| GD-28862 | CTCF | ATTGTGCAGTGCATCGGATT | chr8:128746525-128746547 |
| | | (SEQ ID NO: 3) | |
| GD-28617 | Promoter | GTCAAACAGTACTGCTACGG | chr8:128748014-128748036 |
| | | (SEQ ID NO: 4) | |

### Example 2: Down-Regulation of MYC1 Expression by KRAB Effectors Fused to dCas9sgRNA

This example describes down-regulating MYC1 expression by targeting KRAB effector fused to dCas9sgRNA to the CTCF motif (GD-28616) or the upstream region directly adjacent to the CTCF motif (GD-28859) or the MYC promoter (GD-28617). To target MYC expression in HCC, CRISPR-dCas9 system was modified by tethering it to KRAB.

In this example, dCas9-KRAB mRNA was delivered to human HCC cell lines (HepG2, Hep3B and SKHEP1) with individual sgRNA (Table 1 and FIG. 1A-B) targeting it to the CTCF motif (GD-28616), CTCF “anchor” site (GD-28859), or the MYC promoter (GD-28617). The effector mRNA and sgRNA were co-delivered using LNP delivery with SSOP. For controls, both (1) dCas9 (no effector) combined with sgRNA, and (2) untreated cells were used to assess changes. HCC cells were seeded in 96-well plates in growth media (5000 cell/well). LNP formulations (2-2.5 ug/ml) were then added to the cells to transfect the mRNA and sgRNA. All treatments were done in biological triplicate. Timepoints ranging from 24-168 hours were assessed for MYC expression and cell viability. RNA was isolated from four independent experiments using the RNeasy® plus 96-well Kit (Qiagen) following the Manufacture's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH reference genes using the ΔΔCt method. The untreated and dCas9(no-effector) samples were used as calibrators.

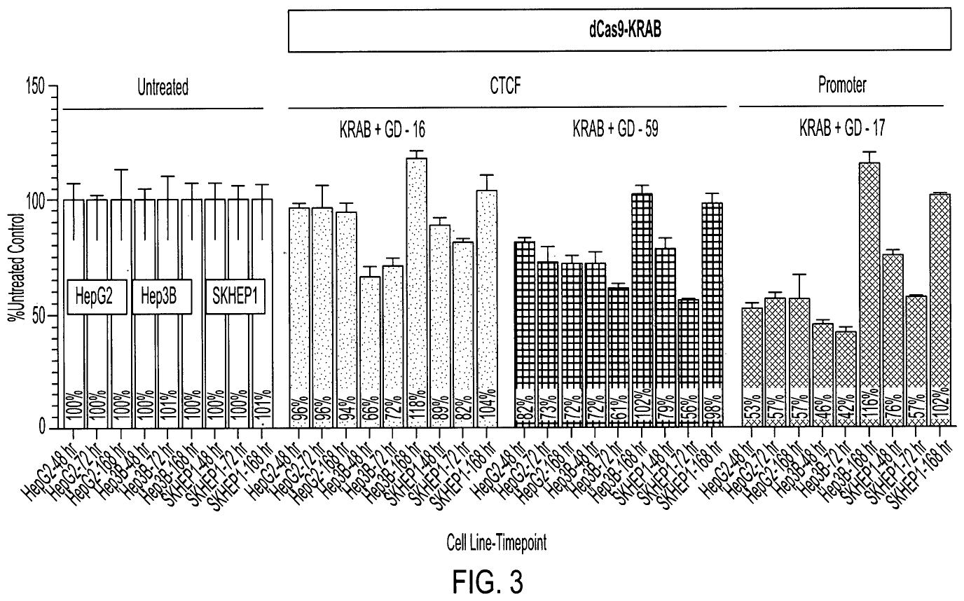

Experimental data demonstrated that LNP-mediated transfection of dCas9-KRAB/GD-28616 down-regulated MYC expression by 11-34% at 48/72-hour timepoints in Hep3B and SKHEP1. LNP-mediated transfection of dCas9-KRAB/GD-28859 downregulated MYC expression by 18-44% at 48/72-hour timepoints in all 3 HCC models (FIG. 3). This effect was reduced in two of the lines (Hep3B and SKHEP1) by 168 hours, but MYC expression remained down by 28% in HepG2 line at 168 hours (FIG. 3). Directing dCas9-KRAB to the MYC promoter via dCas9-KRAB/GD-28617 down-regulated MYC expression by 24-58% at 48/72-hour timepoints in all 3 HCC models. This effect was reduced in two of the lines (Hep3B and SKHEP1) by 168 hours, but MYC remained down by 43% in HepG2 at 168 hours (FIG. 3).

### Example 3: Down-Regulation of MYC1 Expression by KRAB Effectors Fused to Zinc Finger Domains

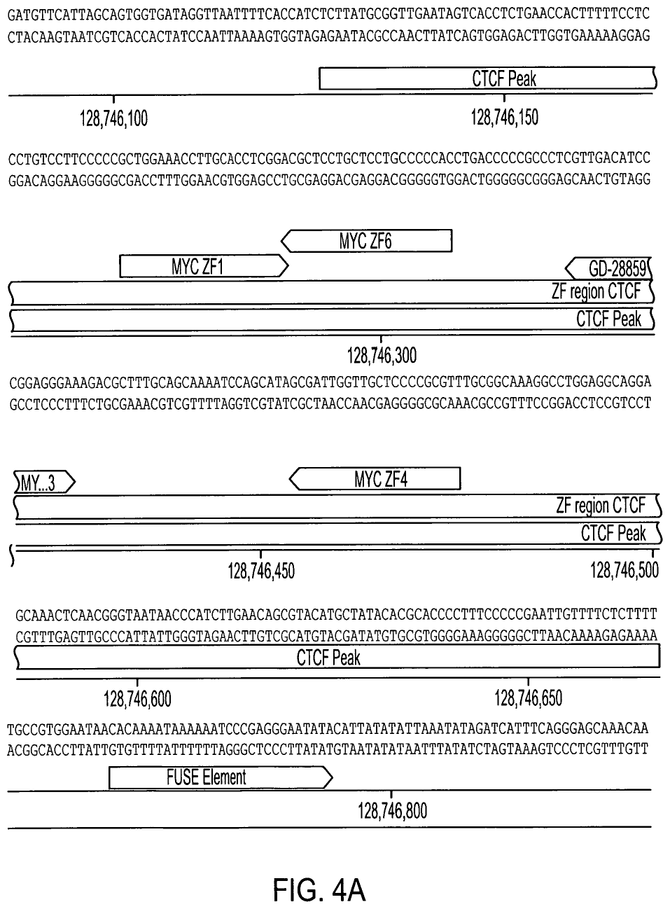

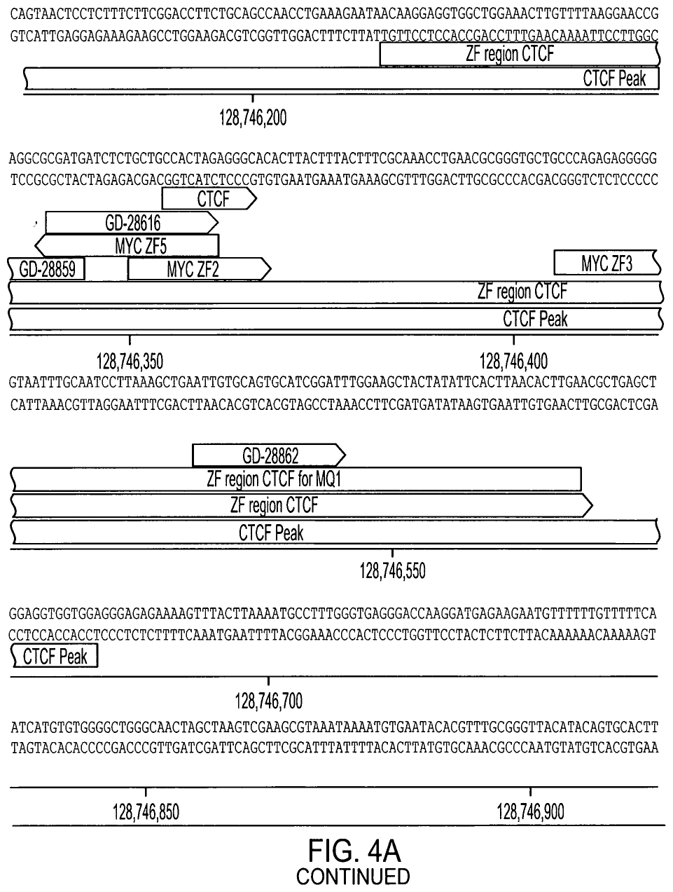

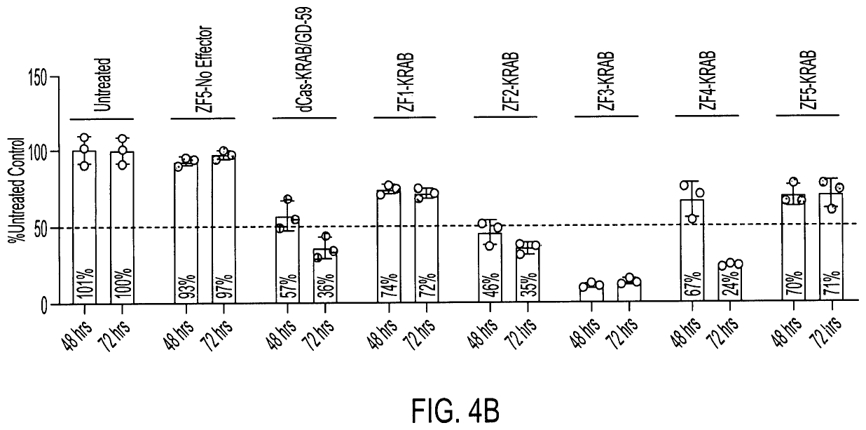

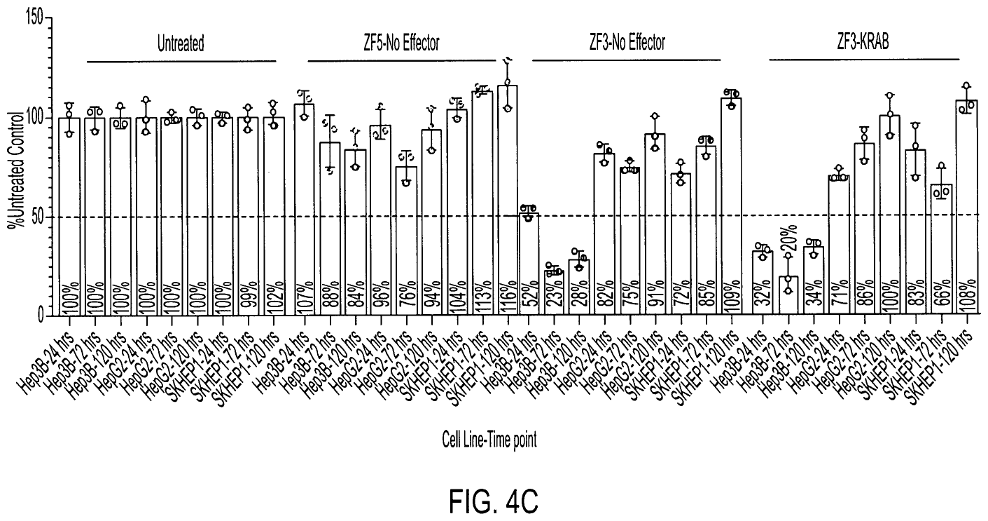

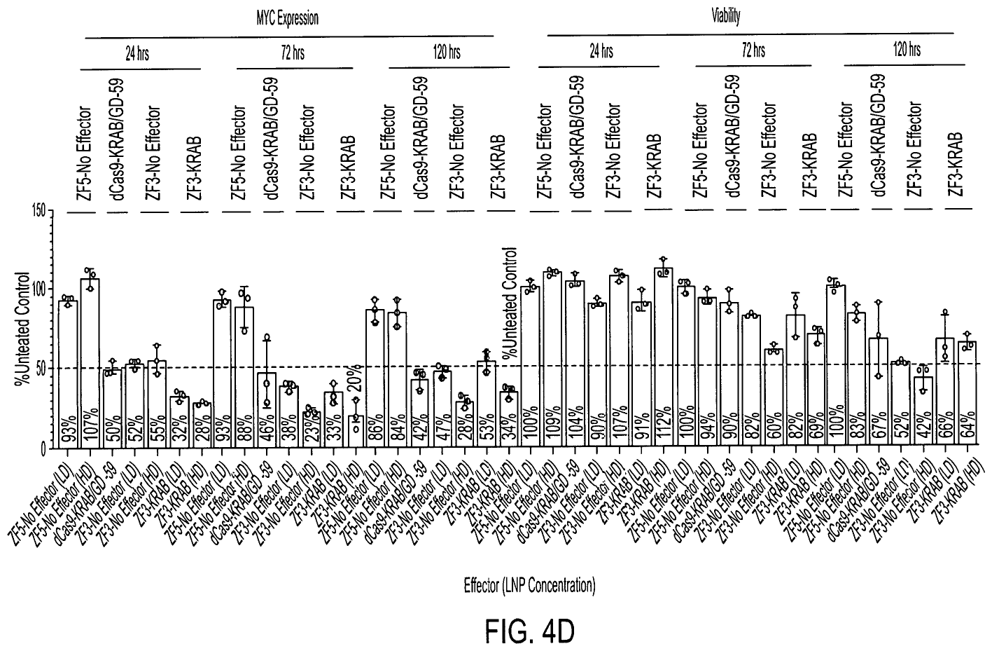

This example describes down-regulating MYC1 expression by targeting KRAB effector fused to zinc finger domains to the upstream region directly adjacent to the CTCF motif (GD-28859). In this example, zinc finger-directed KRAB effectors (ZF-KRAB effectors) were designed to bind the anchor site at the DNA region targeted by GD-28859 (FIG. 4A). 7 constructs (dCas-KRAB/GD-59, ZF1-KRAB, ZF2-KRAB, ZF3-KRAB, ZF4-KRAB, ZF5-KRAB, and ZF6-KRAB) were designed and screened in human HCC models (HepG2, Hep3B, SKHEP1). Negative controls for these experiments included untreated cells and dCas9-KRAB/GD-28859 was used as positive control. RNA was isolated from two independent experiments, each done in biological triplicate, using the Rneasy® Plus 96-well Kit (Qiagen) following the Manufacture's protocol. RNA samples were retrotranscribed to cDNA using LunaScript® RT SuperMix Kit (NEB) and analyzed by quantitative PCR (qPCR) (in technical triplicates) using a MYC specific Taqman primer/probe set assay with the TaqMan™ Fast Advanced Master Mix (Thermo Scientific). MYC expression was quantified relative to the expression of GAPDH reference genes using the ΔΔCt method, the untreated and dCas9(no-effector) samples were used as calibrators. Cell viability was assessed by ATP quantification using CellTiter-GLO® Luminescent Cell Viability Assay (Promega #G9241). HCC cells were seeded in 96-well plates in growth media (5000 cell/well). LNP formulations (0.6-2.5 ug/ml) were then added to the cells to transfect the mRNA and sgRNA. All treatments were done in biological triplicate.

These data showed that ZF2-KRAB, ZF3-KRAB and ZF4-KRAB down-regulated MYC to an equivalent or greater degree than dCas9-KRAB/GD-28859 in Hep3B cells, with ZF3-KRAB having the strongest down-regulatory effect (FIG. 4B). ZF3-KRAB was also shown to down-regulate MYC to an equivalent or greater degree than dCas9-KRAB/GD-28859 in the other two HCC models, HepG2 and SKHEP1 (FIG. 4C). ZF3-No Effector (NE) was also determined to have a down-regulatory effect on MYC expression, likely due to steric blocking of a regulatory site. Both ZF3-KRAB and ZF3-NE in Hep3B demonstrated downregulatory effects on MYC expression and viability over a time-course of 24, 72 and 120 hours (FIG. 4D).

### Example 4: Down-Regulation of MYC1 Expression by MQ1 Effectors Fused to dCas9sgRNA

This example describes down-regulating MYC1 expression by targeting MQ1 effector fused to dCas9sgRNA to the CTCF motif (GD-28616) or the upstream region directly adjacent to the CTCF motif (GD-28859), GD-28862, or the MYC promoter (GD-28617).

In this example, dCas9-MQ1 mRNA was delivered to human HCC cell lines (HepG2, Hep3B and SKHEP1) with individual sgRNA (Table 11 and FIG. 1A-B) targeting it to the CTCF “anchor” site, or the MYC promoter. The effector mRNA and sgRNA were co-delivered using LNP delivery with SSOP. For controls, both (1) dCas9 (no effector) combined with sgRNA, and (2) untreated cells were used to assess changes. The protocol essentially as described in Example 2 was followed to conduct the experiment.

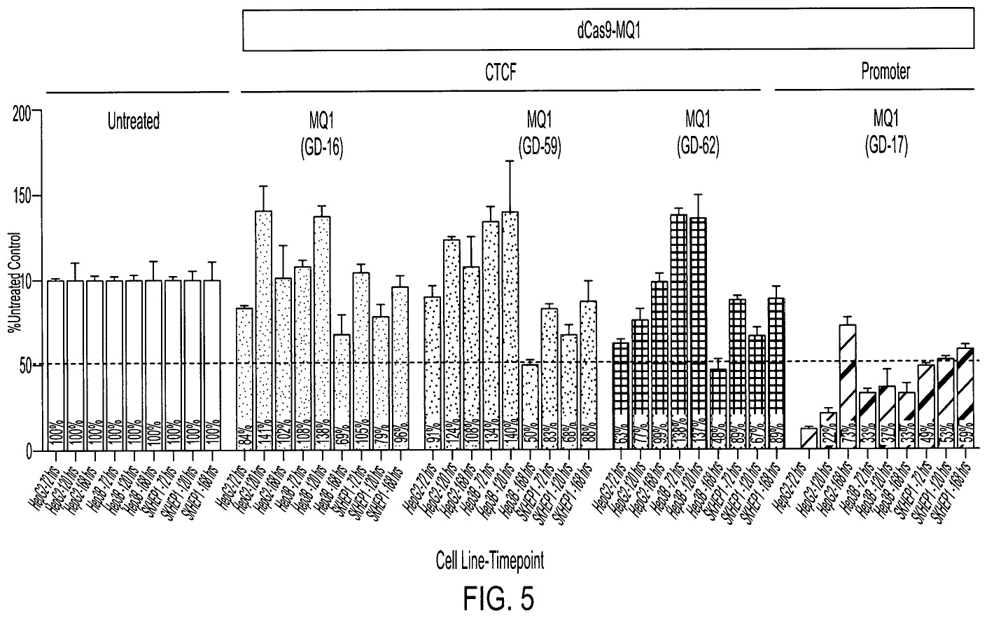

Results showed that dCas9-MQ1 targeted to the CTCF by GD-16, GD-59 and GD-62, led to variable down-regulation and up-regulation in a cell line specific manner. dCas9-MQ1 targeted to the promoter by GD-17 resulted in 50-90% MYC down-regulation at 72 hours and persisted out to 168 hours in all three HCC models (FIG. 5). MYC down-regulation dramatically decreased viability in HepG2 and Hep3B at 72 and 168 hours, although SK-HEP-1 viability was minimally affected by MYC down-regulation. The dCas9-sgRNA controls had no effect on expression or viability as compared to the untreated controls

### Example 5: Down-Regulation of MYC1 Expression by MQ1 Effectors Fused to Zinc-Finger Domains

This example describes down-regulating MYC1 expression by targeting MQ1 effector fused to Zinc-Finger domains to the MYC promoter (GD-28617).

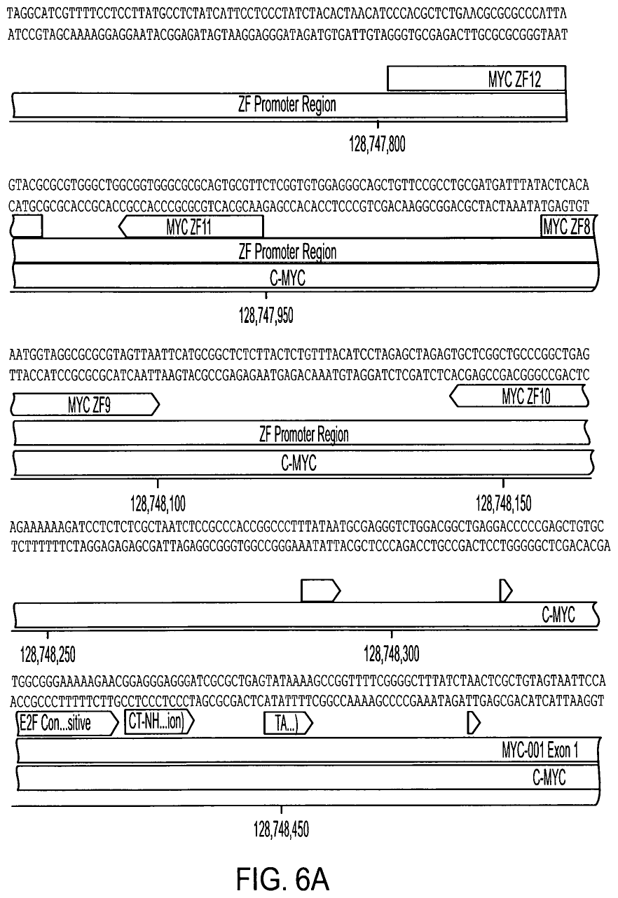

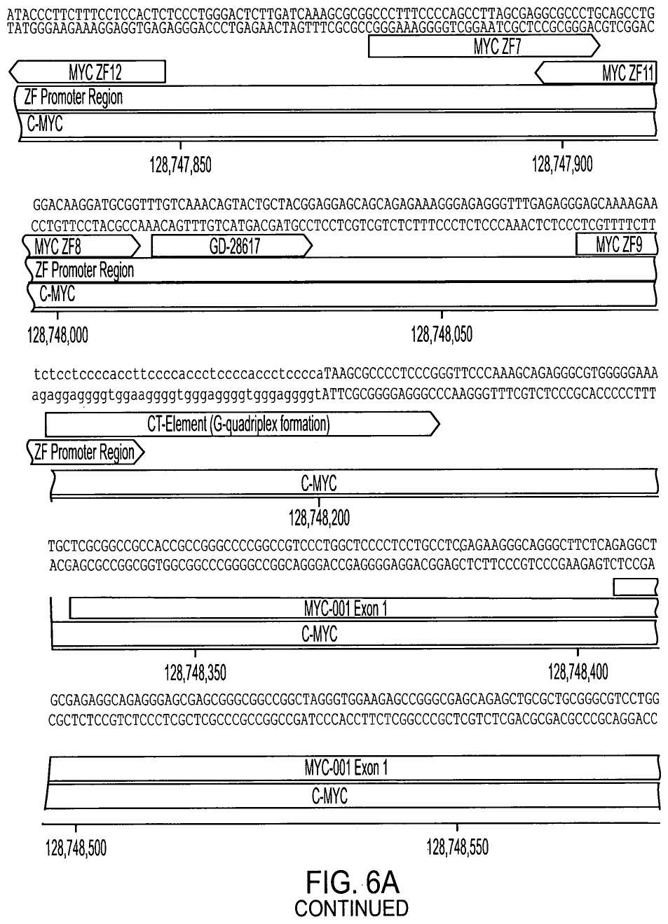

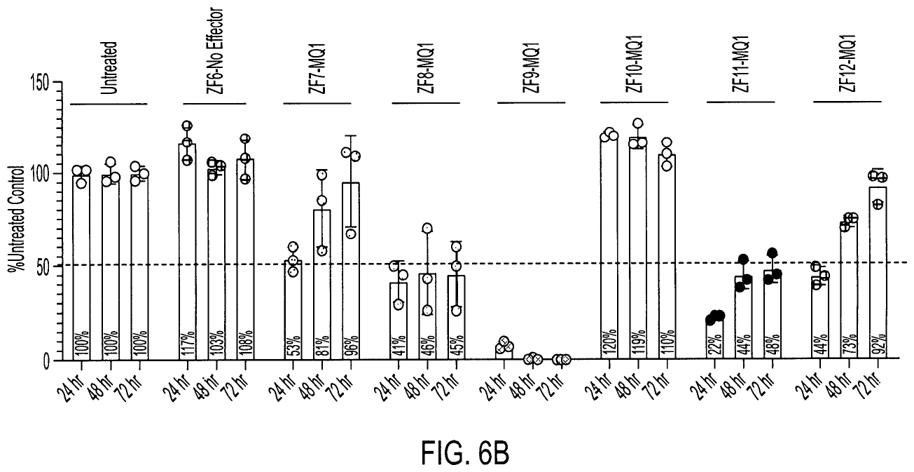

In this example, zinc finger-directed MQ1 effectors (ZF-MQ1 effectors) were designed to bind the DNA region targeted by GD-28617 (FIG. 4A and FIG. 6A). 6 constructs (ZF7-MQ1, ZF8-MQ1, ZF9-MQ1, ZF10-MQ1, ZF11-MQ1, and ZF12-MQ1) were designed were designed around GD-28617 to bind the region identified in Example 4 screening and screened in human HCC models (HepG2, Hep3B, SKHEP1). Untreated cells and cells transfected with ZF protein alone (ZF-no effector or ZF-NE) were used as the negative control for the experiment and dCas9-KRAB/GD-28859 was used as positive control. The protocol as described in Example 3 was followed.

Results demonstrated, ZF8-MQ1, ZF9-MQ1 and ZF11-MQ1 down-regulated MYC to the greatest degree in Hep3B cells, with ZF9-MQ1 having the strongest down-regulatory effect (FIG. 6B). The data further demonstrated that the genomic binding sites of ZF-MQ1 molecules that successfully reduced MYC expression (ZF8-MQ1, ZF9-MQ1 and ZF11-MQ1) were in close proximity with to the ZF-MQ1 molecules that did not alter MYC expression (ZF7-MQ1, ZF10-MQ1 and ZF12-MQ1). This could be an effect of binding efficiency, 3-D orientation of binding, extend of methylation of the region, or a result of specific effector positioning in relation to regulatory elements in the MYC promoter. It appears that slight shifting the ZF-effector binding site has significant consequences on MYC gene regulation. ZF9-MQ1 was found to strongly down-regulate MYC mRNA and reduce viability in all three HCC models, and that the effects seen with the ZF9-MQ1 construct were much greater than what can be directed by the dCas9-MQ1/GD-28617 system.

### Example 6: ZF9-MQ1 Demonstrates the Strongest Downregulatory Effect on MYC1 Expression Compared to Other ZF-MQ1 Effectors

This example describes ZF9-MQ1 has the strongest effect on down-regulating MYC1 expression by targeting MQ1 effector fused to Zinc-Finger domains to the MYC promoter (GD-28617) compared to other ZF-MQ1 effectors tested.

In this example, the effect of ZF9-MQ1 on downregulating MYC expression in all HCC models (HepG2, Hep3B and SKHEP1) were compared against that of ZF12-MQ1, ZF8-MQ1, and dCas9-MQ1/GD-28617 respectively. Untreated cells and cells transfected with ZF protein alone (ZF-no effector or ZF-NE) were used as the negative control for the experiment and dCas9-KRAB/GD-28859 was used as positive control. The protocol as described in Example 3 was followed.

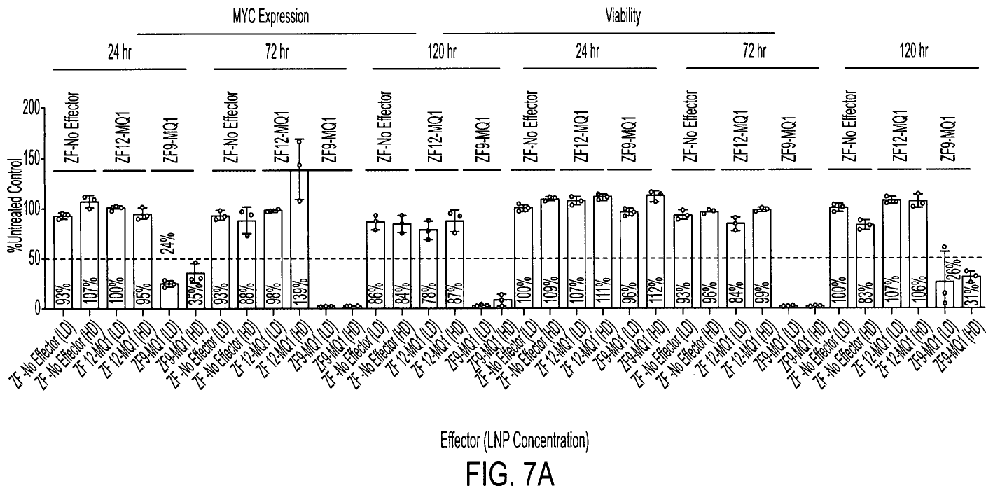

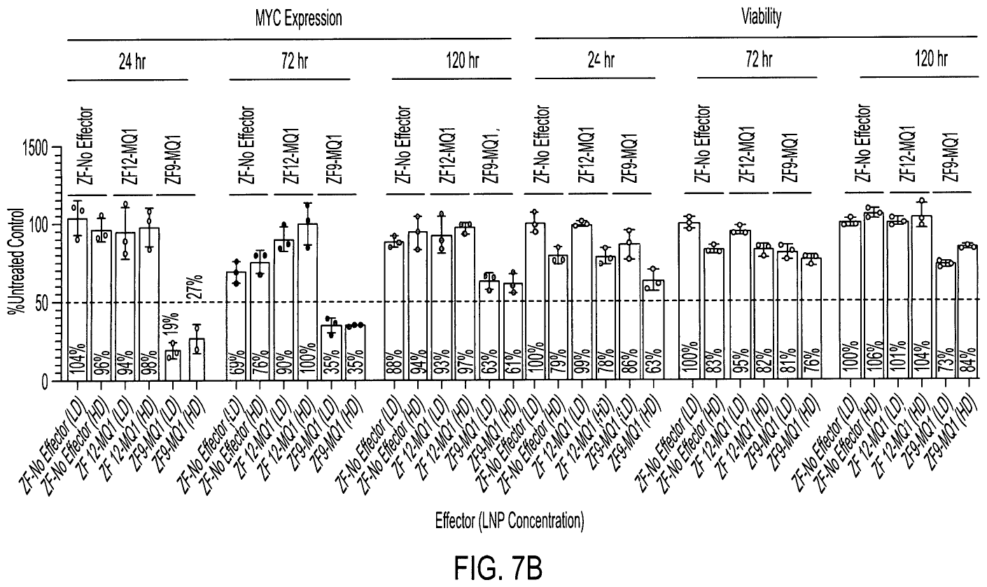

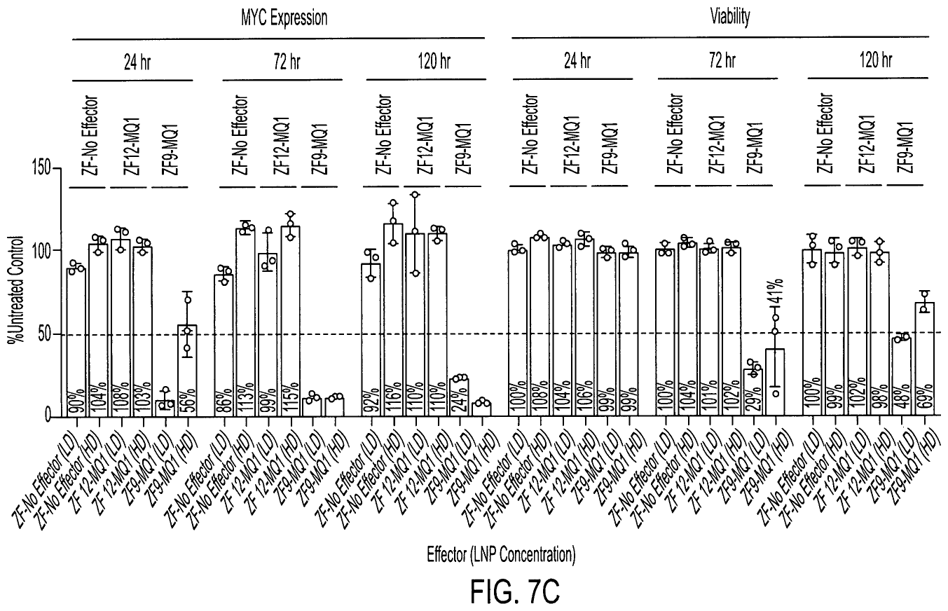

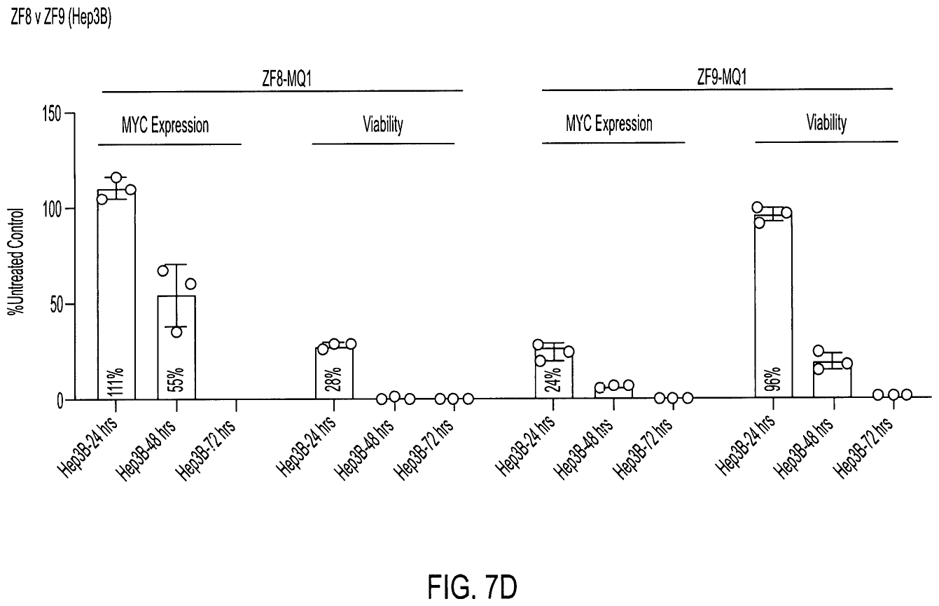

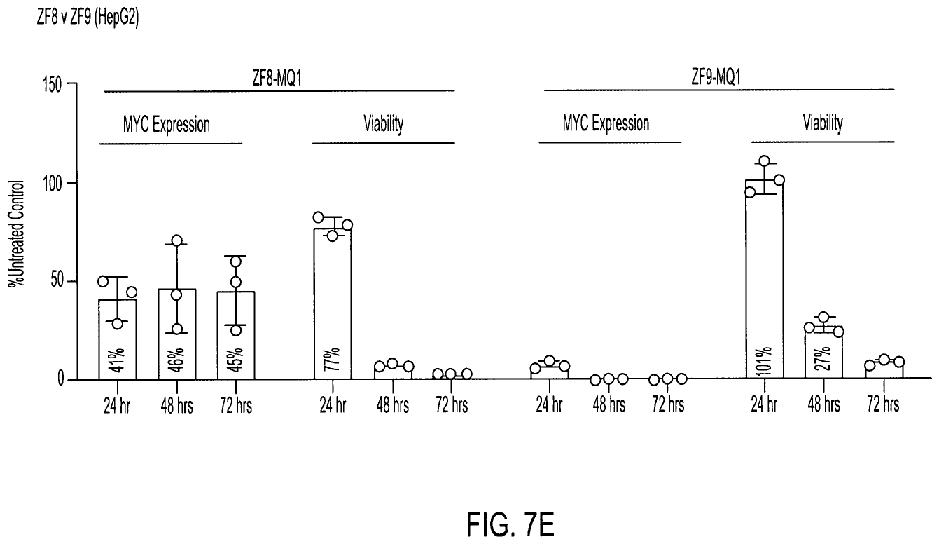

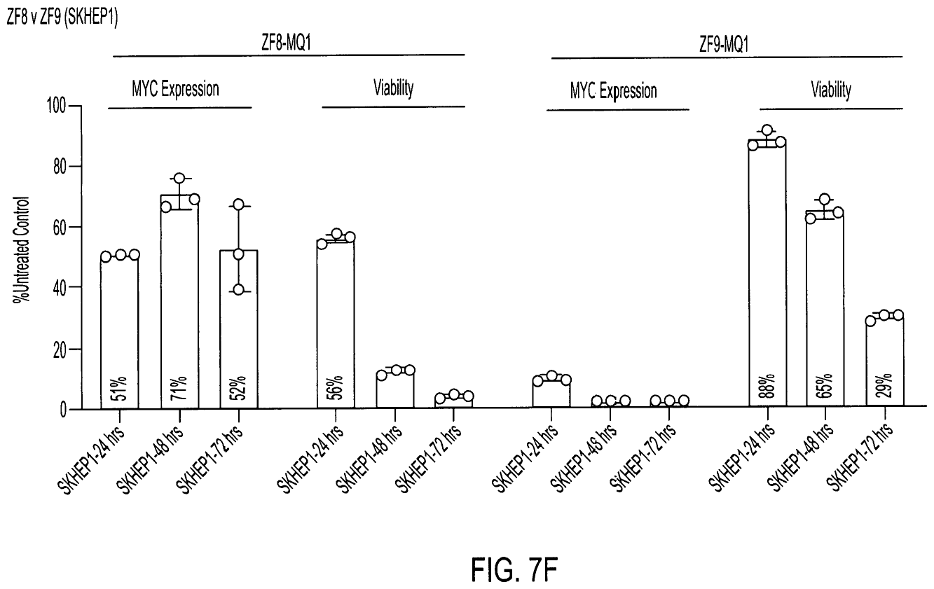

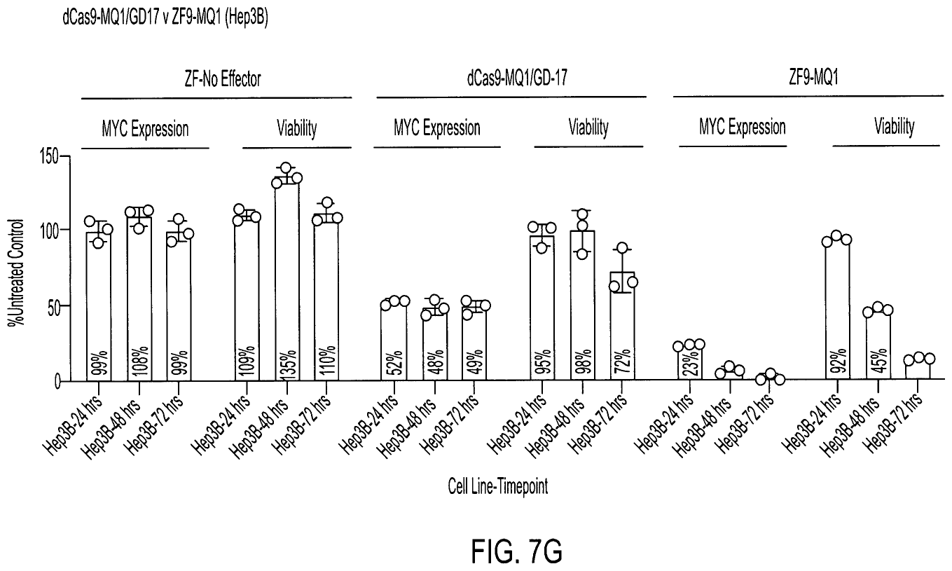

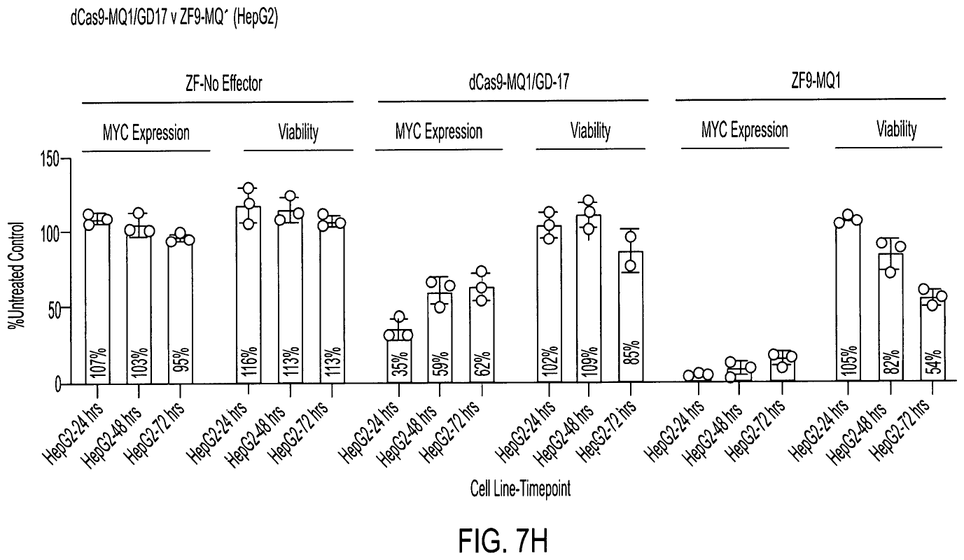

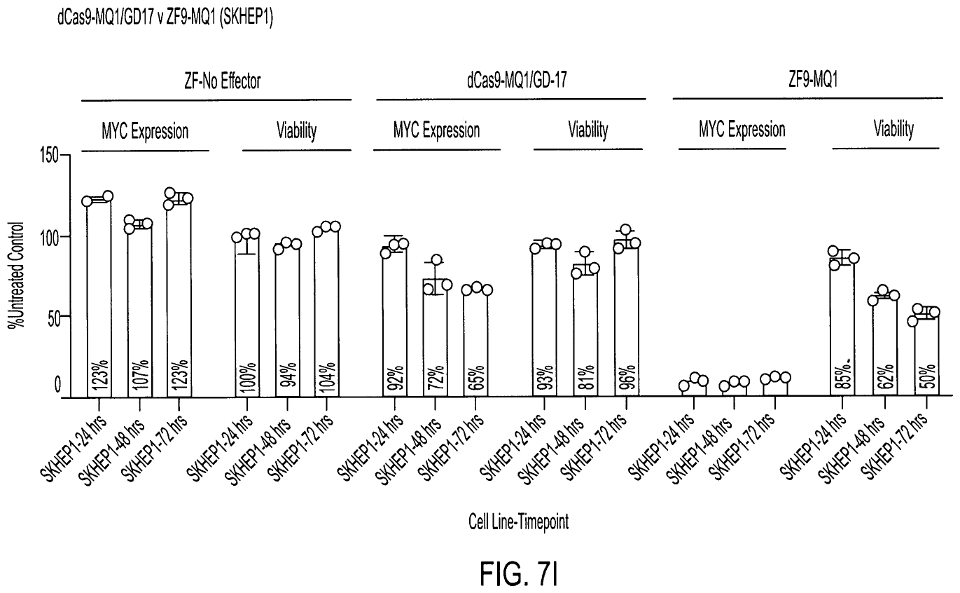

ZF9-MQ1 was shown to strongly down-regulate MYC and reduce viability in all three HCC models examined (Hep3B, HepG2 and SKHEP1) compared to ZF12-MQ1 (FIG. 7A-C). ZF9-MQ1 regulated MYC1 expression downregulation is comparatively greater than the downregulation seen in presence of ZF8-MQ1 (FIG. 7D-F) and that the effects seen with the ZF9-MQ1 construct are much greater than what can be directed by the dCas9-MQ1/GD-28617 system (FIG. 7G-I). The data further showed, while ZF9-MQ1 down-regulated MYC expression as quickly as 24 hours, the effects on viability were not observed until 72 hours or later. In contrast, ZF8-MQ1 effected a much less significant change in MYC mRNA, and a much more immediate reduction in cell viability.

### Example 7: dCas9-MQ1 has a Significantly Greater Effect on MYC Expression than Human dCas9-DNMT1 or dCas9-DNMT-3A-3L

This example demonstrates that dCas9-MQ1 has a significantly greater effect on MYC expression than the human dCas9-DNMT1 or dCas9-DNMT-3A-3L.

In this example, the CRISPR-dCas9 system was modified by tethering it to a selection of epigenetic repressors, including KRAB, human DNMT1, a human DNMT3A-3L fusion, human DNMT3Bm prokaryotic DNMT, and MQ1. These molecules modulate transcriptional repression by recruiting repressive complexes (KRAB) or methylating the CpG nucleotides of the DNA (DNMT1, DNMT3A-3L, DNMT3B and MQ1). A panel of human HCC cell lines, including HepG2, Hep3B and SKHEP1, were utilized to carry out effector screening in hepatoma models. GD-28617 sgRNA was combined with dCas9-DNMT mRNA and was co-delivered by LNP transfection into these cell lines. CRISPR-Cas9 studies assessing the efficacy of LNP delivery by measuring editing efficiency of sgRNA/Cas9 confirmed 90-99% editing efficiency using this system (data not shown). Following transfection, cells were then assayed for MYC mRNA expression by qPCR, and for viability by CellTiter-GLO®, at multiple timepoints. For targeted and global methylation analysis by Bisulfite sequencing genomic DNA was isolated using Lucigen DNA extraction kit.

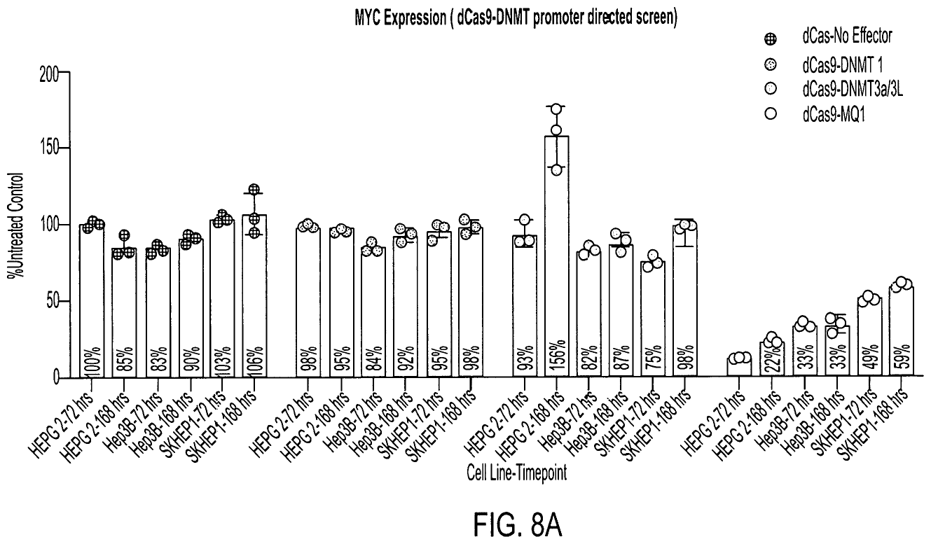

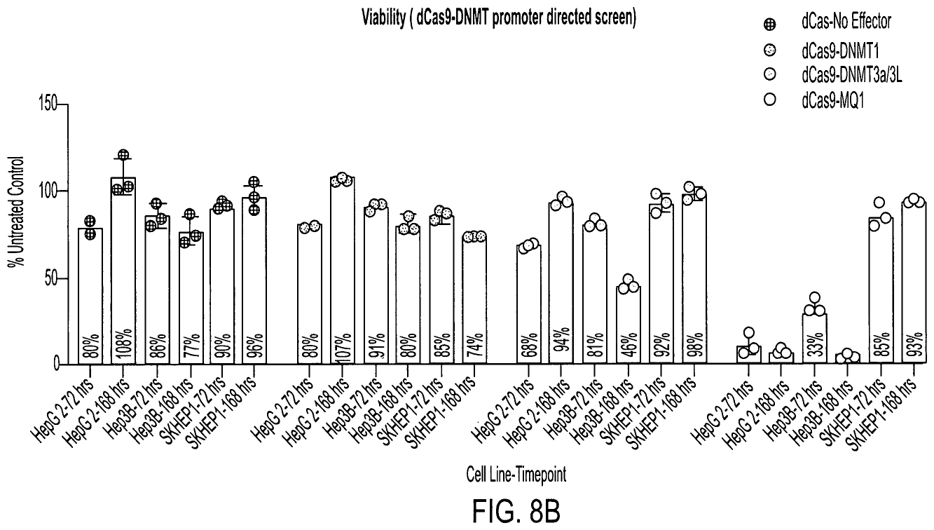

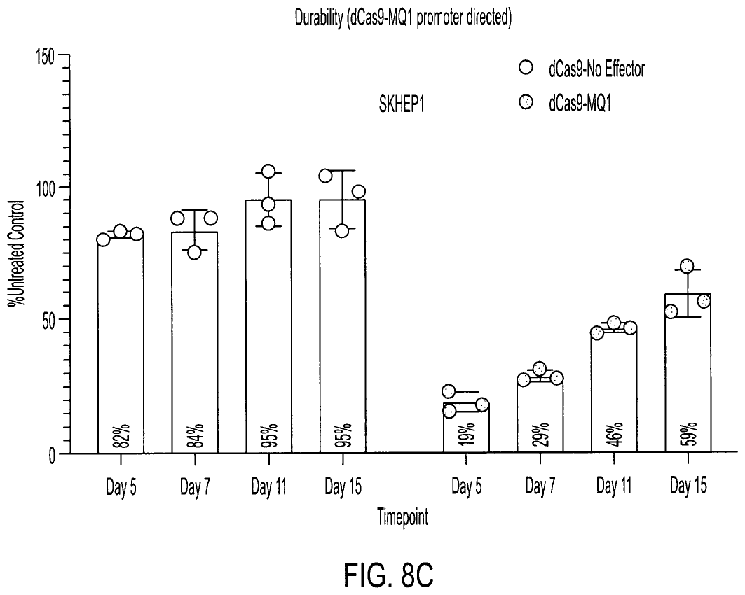

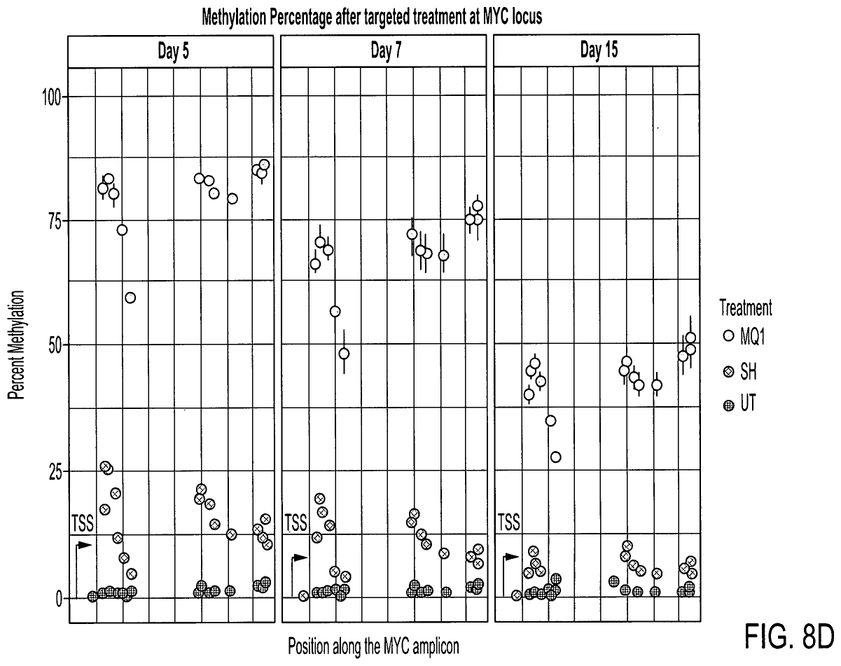

These studies demonstrate that dCas9-MQ1 has a significantly greater effect on MYC expression than any of the human dCas9-DNMTs examined or dCas9-KRAB (data not shown). dCas9-MQ1 effected a 50-90% decrease in mRNA at 72 hours across the three lines (FIG. 8A). MYC down-regulation dramatically decreased viability in HepG2 and Hep3B at 72 and 168 hours, although SK-HEP-1 viability was minimally affected by MYC down-regulation (FIG. 8B). The dCas9-sgRNA controls had no effect on expression or viability as compared to the untreated controls.

As the SK-REP-1 model demonstrated minimal changes to viability upon MYC down-regulation, this line was utilized to assess the durability of the down-regulatory effect on MYC expression. At day 5, MYC mRNA was decreased by 80% as compared to the dCas9 DBD only and untreated controls. At day 7 and day 11, MYC mRNA was decreased ˜70% and ˜55% respectively. As far out as day 15, an ˜40% down-regulation in transcript was maintained (FIG. 8C). Using bisulfite genomic sequencing, a qualitative and quantitative means of measuring 5-methylcytosine at a single base pair resolution, it was established that treatment with dCas9-MQ1/GD-28617 directs de-novo methylation to the targeted region and that these transcriptional changes tightly correlate with the percentage of CpG methylation in the target region and confirm methylation persists out to day 15 (FIG. 8D).

### Example 8: Treatment with dCas9-MQ1/GD-17 Inhibit Tumor Growth In Vivo

This example describes in vivo analysis of dCas9-MQ1/GD-17 treatment of a subcutaneous Hep3B xenograft, resulting in inhibition of tumor growth as compared to control treatments (PBS and/or dCas9/GD-SafeHarbor).

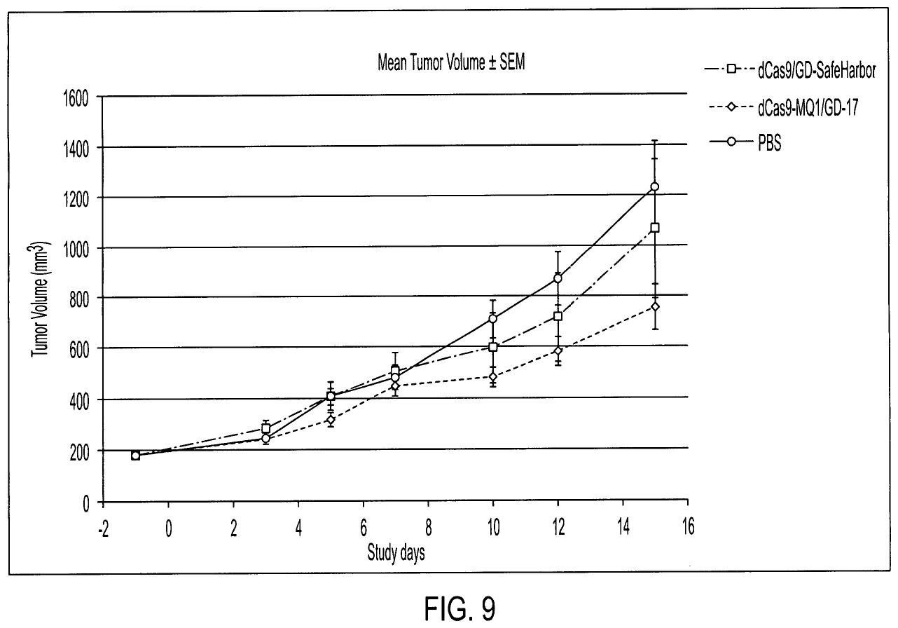

In this example, 0.6 mg/ml LNP (MC3) formulated effector (dCas9-MQ1/GD-17) was delivered to tumor sites by intratumoral injection on day 1 and day 7 (20 μl/mice) in the test group animals. The control group mice were injected at the tumor site either with PBS or with 0.6 mg/ml LNP (MC3) control effector dCas9/GD-SafeHarbor (20 μl/mice). Each control and test group consisted of 6 animals having SubQ Hep3B Xenografts (250 mm³). Changes in tumor volume for each group was measured every 3 days for 15 days. At the end of 15 days, the changes in mean tumor volume were measured using Paired T-test and was plotted. Mice treated with dCas9-MQ1/GD-17 showed reduction in tumor volume compared to both the control groups (FIG. 9).

### Example 9: dCas9-MQ1/GD-17 Down-Regulates MYC in the Context of Hepatitis B Infection in Human Hepatocytes

This example demonstrates dCas9-MQ1/GD-17 down-regulates MYC in the context of hepatitis B infection in human hepatocytes.

In this example, human hepatocyte cells were infected with HBV and both uninfected and infected hepatocyte cells were plated and grown over an 8-day period. At the end of 9 days, HBV infected human hepatocyte cells showed higher MYC expression compared to the uninfected cells. Both the uninfected and HBV infected hepatocyte cells were transfected by LNPs with control effector (dCas9+SafeHarbor sgRNA (GD-SH)), or the effector dCas9-MQ1/GD-17 and were allowed to grow for another 48 hours. MYC expression was then assessed by qPCR after 48 hours (FIG. 8). The study was done in biological triplicate. RNA was isolated using the Rneasy® Plus 96-well Kit (Qiagen) following the Manufacture's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH reference genes using the ΔΔCt method, and dCas9+GD-SH samples were used as calibrators.

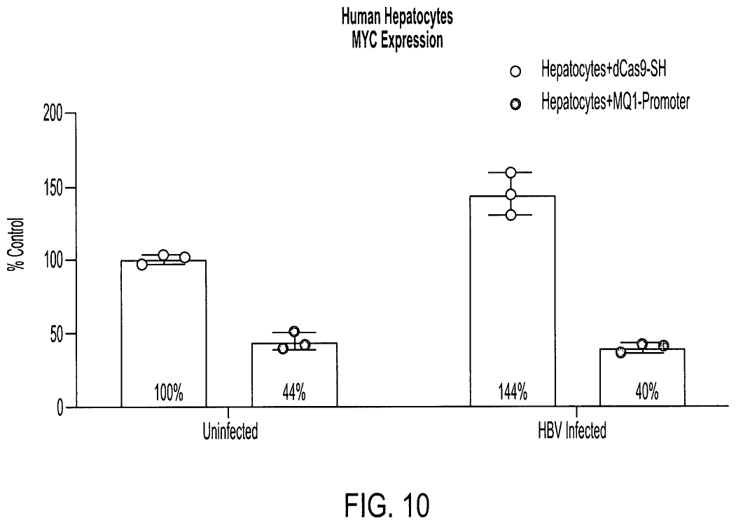

MYC expression, as assessed by qPCR after 48 hours and normalized to uninfected human hepatocyte controls demonstrated that dCas9-MQ1/GD-17(promoter) down-regulates MYC in uninfected and infected cells (FIG. 10).

### Example 10: Combination of Zinc Finger Domains Plus Transcriptional Effectors Targeted to MYC ASMC Down-Regulate MYC Expression

This example demonstrates targeting a KRAB effector (or no-effector or NE) fused to a zinc-finger domain to the upstream region directly adjacent to the CTCF motif (ZF3-NE or ZF3-KRAB) and targeting an MQ1 effector fused to a Zinc-Finger domain to the MYC promoter (ZF9-MQ1) downregulates MYC mRNA expression. A combination of ZF9-MQ1 and ZF3-KRAB is more effective in downregulating MYC expression compared to the other effectors tested in this Example, individually or in combination.

ZF3-NE or ZF3-KRAB effectors targeting the anchor CTCF were combined with ZF9-MQ1 designed to bind and target the MYC promoter in the HCC cells line, Hep3B. dCas-KRAB/GD-28859 and dCas9-MQ1/GD-28617 were used at positive controls for the two regions. Negative controls for these experiments included untreated cells and cells transfected with ZF5-NE and green-fluorescent protein (GFP). The effector mRNAs were co-delivered using LNP delivery with SSOP. HCC cells were seeded in 96-well plates in growth media (5000 cell/well). LNP formulations (0.6 ug/ml) were then added to the cells to transfect the mRNA then incubated for different time points. RNA was isolated from three biological replicates, using the Rneasy® Plus 96-well Kit (Qiagen) following the Manufacture's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH reference genes using the ΔΔCt method, the untreated and dCas9(no-effector) samples were used as calibrators.

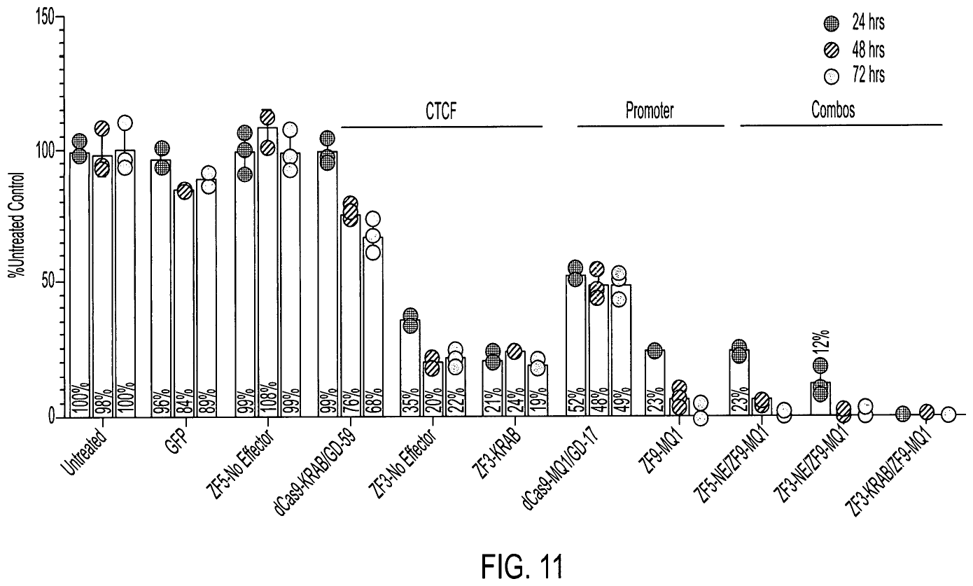

These data showed that ZF3-KRAB plus ZF9-MQ1 down-regulated MYC to a greater degree than dCas9-KRAB/GD-28859, ZF3-KRAB, ZF3-NE, dCas9-MQ1/GD-28617, ZF9-MQ1 alone or ZF3-NE or ZF5-NE plus ZF9-MQ1 combination (FIG. 11). GFP and ZF5-NE alone had negligible effect on MYC expression. The data was comparable over the time course tested (24, 48 and 72 hours) with maximum repression after 24 hours continuing to at least 72 hours.

### Example 11: Combination of ZF9-MQ1 and ZF3-KRAB Down-Regulates MYC Expression in Three HCC Models

This example demonstrates that a combination of ZF9-MQ1 and ZF3-KRAB downregulates MYC mRNA expression more in additional HCC cell lines (Hep3B, HepG2 and SKHEP1) compared to other effectors tested alone or in combination.

ZF3-NE or ZF3-KRAB effectors targeting the anchor CTCF were combined with ZF9-MQ1 designed to bind and target the MYC promoter in three HCC cells line, Hep3B, HepG2 and SKHEP1. Negative controls for these experiments included cells transfected with ZF5-NE. The effector mRNAs were co-delivered using LNP delivery with SSOP. HCC cells were seeded in 96-well plates in growth media (5000 cell/well). LNP formulations (0.6 ug/ml) were then added to the cells to transfect the mRNA then incubated for different time points. RNA was isolated from three biological replicates, using the Rneasy® Plus 96-well Kit (Qiagen) following the manufacturer's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH reference genes using the ΔΔCt method, the untreated and dCas9(no-effector) samples were used as calibrators.

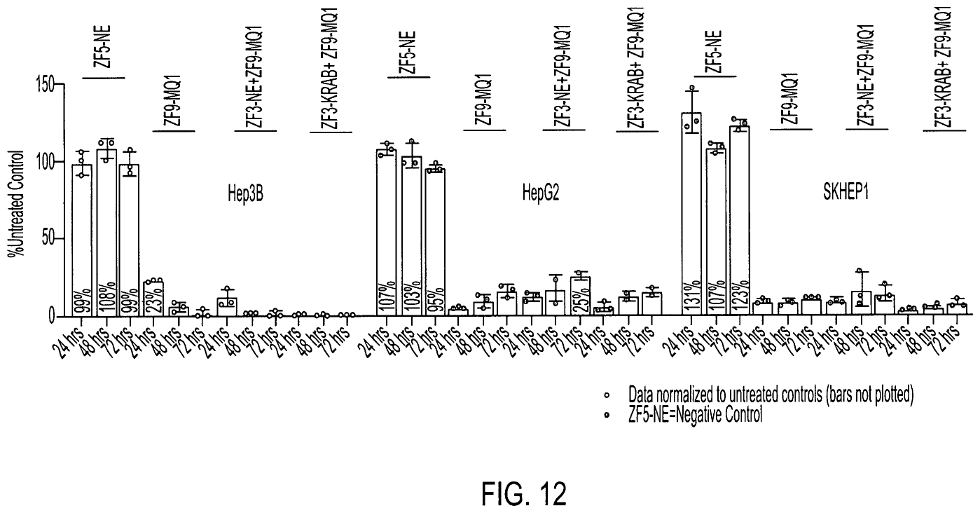

These data showed that ZF3-KRAB plus ZF9-MQ1 downregulated MYC to a greater degree than ZF9-MQ1 alone or ZF3-NE plus ZF9-MQ1 combination (FIG. 12). The data was comparable over the time course tested (24, 48 and 72 hours).

### Example 12: Dose Response Curves of Viability and MYC Expression for ZF9-MQ1 in Additional HCC Models

This example describes down-regulating Myc expression and cell viability by ZF9-MQ1 using dose response curves in five HCC cell lines.

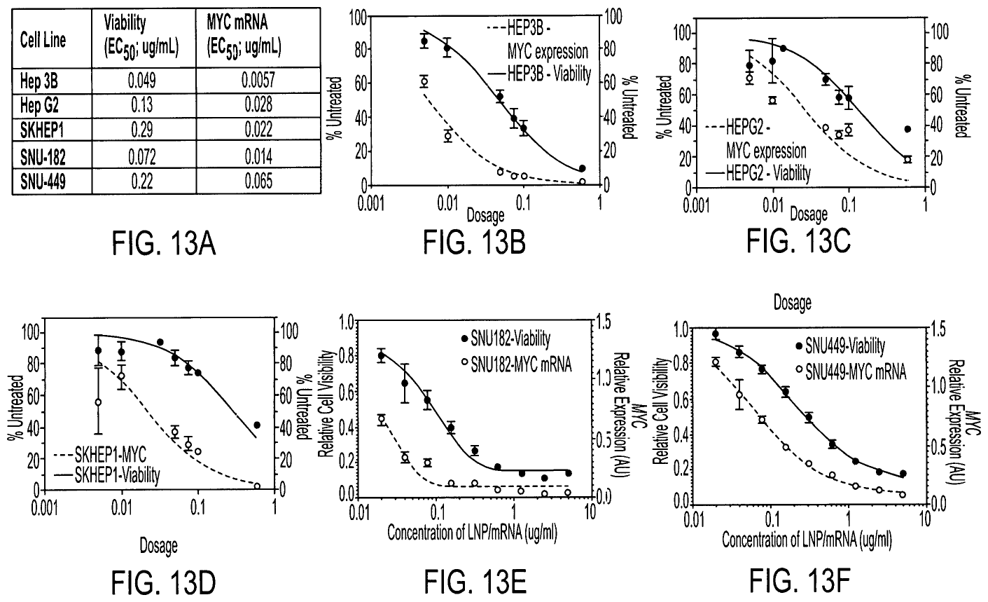

In this example ZF9-MQ1 designed to bind and target the MYC promoter was dosed at multiple concentration in five HCC cells line, Hep3B, HepG2, SKHEP1, SNU-182 and SNU-449 (FIG. 13A). d Untreated cells were used as negative controls. The effector mRNA was delivered using LNP delivery with SSOP. HCC cells were seeded in 96-well plates in growth media (5000 cell/well). LNP formulations (starting at 5 or 0.6 ug/ml) were then added to 3 wells each then diluted ˜1:2 in subsequent wells for 6 to 10 doses in order to transfect mRNA then incubated for 72 hours. Different replicate plates were collected for viability and RNA. Viability was measured using the Celltiter GLO assay kit from Promega according to manufacturer's protocol. RNA was isolated from three biological replicates, using the Rneasy® Plus 96-well Kit (Qiagen) following the Manufacturer's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH reference genes using the ΔΔCt method, the untreated and dCas9(no-effector) samples were used as calibrators. The EC50 value was calculated from the dose response curve.

These data showed that ZF9-MQ1 downregulated MYC expression and reduced viability in all five HCC cell lines tested. ZF9-MQ1 down-regulated MYC expression with a range of EC50 between 0.0057-0.065 ug/ml LNP/mRNA with a EC50 effect on viability ranging from 0.049-0.29 ug/ml in vitro at 72 hours in these five HCC cell lines. (FIG. 13B-F).

### Example 13: In Vivo Efficacy of ZF9-MQ1 in Hep 3B Model Grown Subcutaneously in Nude Mice

This example demonstrates that treatment with ZF9-MQ1 inhibited the growth of Hep 3B tumors established in female nude mice.

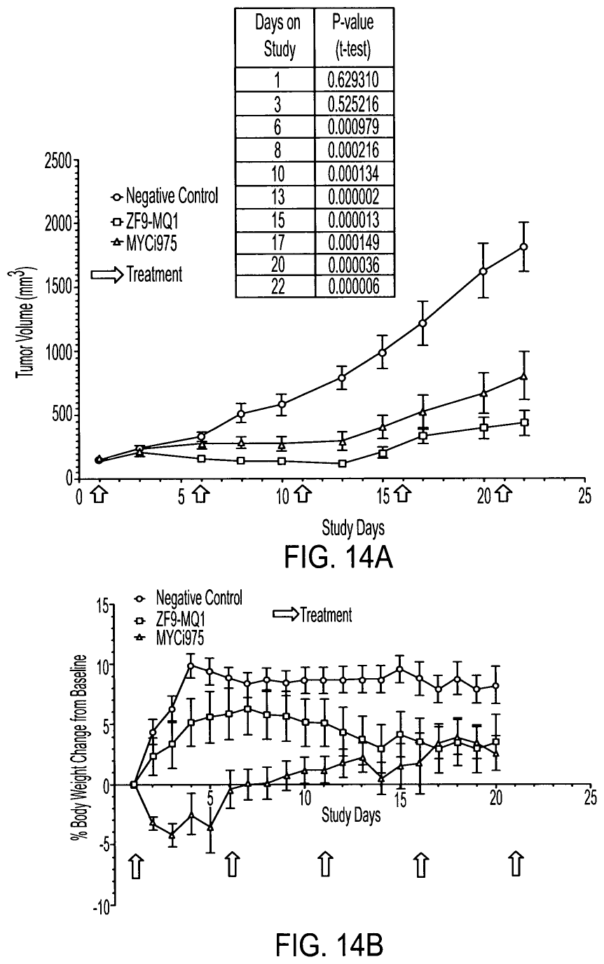

Hep 3B tumor cells were implanted into the left flank of thirty female nude mice to induce tumor. Changes in tumor volume was measured and the treatment initiated when mean tumor volume reached approximately 100-150 mm³. Mice were divided into treatment and control groups so that mean tumor volume in each group were approximately equal. Control groups were injected either with PBS or with MYCi975, a small molecule comparator. The mice in each group were injected intratumorally with PBS (every 5 days for 3 doses then switched to IV for 2 doses), intravenously with ZF9-MQ1 (every 5 days at 3 mg/kg) or intraperitoneally with MYCi975 (once daily for 5 days/week). All animals were weighed daily and assessed visually. Changes in tumor volume for each group were measured 3 times per week. Over the course of 22 days, the changes in body weight from baseline and the mean tumor volume were measured using Paired T-test and was plotted (FIG. 14A-B).

The results show that ZF9-MQ1 was able to significantly reduce tumor growth (from day 6 forward) when compared to PBS control treated mice. ZF9-MQ1 reduced tumor growth more than the small molecule comparator (MYCi975) (FIG. 14A). In addition, IHC staining 48 hours after the last dose showed expression of the ZF9-MQ1 polypeptide, a decrease in MYC expression, and a decrease in proliferation as measured by Ki67 (data not shown). ZF9-MQ1 had minimal effect on overall animal weight (FIG. 14B).

### Example 14: In Vivo Efficacy of ZF9-MQ1+ZF3-KRAB in Hep 3B Model Grown Orthotopically in Fox Chase CB17 SCID Mice

This example demonstrates the long-term anti-tumor efficacy and durability of bi-cistronic ZF9-MQ1+ZF3-KRAB following dosing, in the orthotopic Hep3B-luc model in female Fox Chase CB17 SCID mice.

Hep-3B-luc cells were injected in the upper left lobe of the liver in SCID mice. The mean ventral view whole body tumor-associated bioluminescence (TABL) for each group at randomization was ˜2.8×109 p/s. Mice were randomly allocated to two groups of 12 mice each for treatment with PBS and ZF9-MQ1+ZF3-KRAB and one group of 6 mice for treatment with sorafenib on day 7 of the post-implantation of the cells. Treatment started on day 8 post-implantation of tumor cells (marked as day 1 of dosing on graph). Mice were treated intravenously with PBS (every 5 days for 4 doses, then every 3 days for 2 doses), intravenously with LNP (MC3) ZF9-MQ1+ZF3-KRAB (1.5 mg/kg every 5 days for 2 doses, 3 mg/kg every 5 days for 3 doses, 3 mg/kg every 3 days for 1 dose), orally with sorafenib (50 mg/kg daily). All animals were weighed daily and assessed visually. Tumor size were measured by bioluminescence 2 times per week.

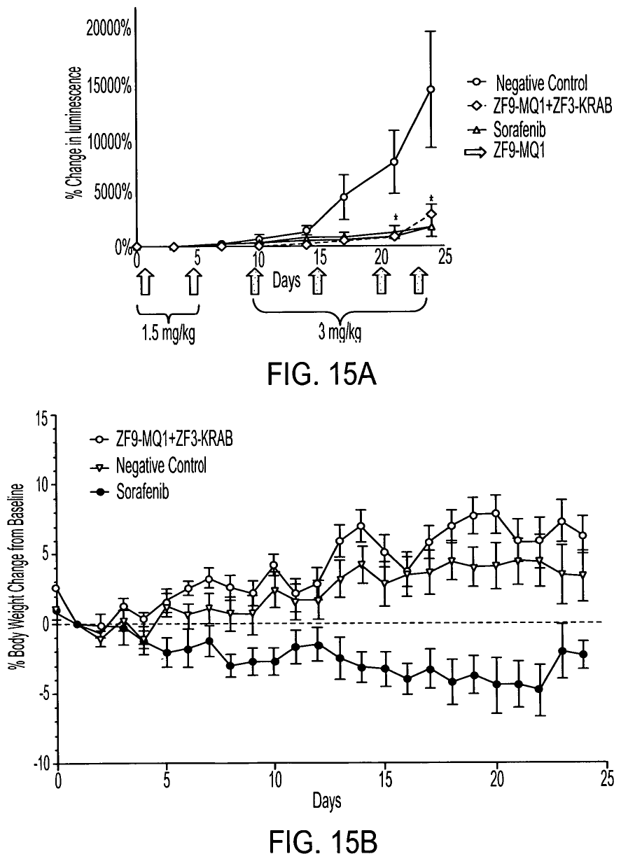

The results show that treatment with ZF9-MQ1+ZF3-KRAB significantly reduced tumor growth (from day 21 forward) when compared to PBS control treated mice. ZF9-MQ1+ZF3-KRAB reduced tumor growth was comparable to soraterub (FIG. 15A). Treatment with ZF9-MQ1+ZF3KRAB had minimal effect on overall animal weight (FIG. 15B).

### Example 15: In Vivo Efficacy of ZF9-MQ1 and Co-Formulated ZF9-MQ1+ZF3-KRAB in Hep 3B Model Grown Subcutaneously in Nude Mice

This example demonstrates that ZF9-MQ1 and co-formulated ZF9-MQ1+ZF3-KRAB was able to inhibit the growth of Hep 3B tumors in a dose-dependent manner in female nude mice.

Hep 3B tumor cells were implanted into the left flank of seventy-two female nude mice to induce tumor. Changes in tumor volume was measured and the treatment initiated when mean tumor volume reached approximately 200 mm³. Mice were divided into nine treatment groups (8 mice each) so that mean tumor volume in each group was approximately equal. The mice in each group were injected intravenously with PBS (every 5 days for 3 doses then every 3 days for 3 doses), ZF9-MQ1 at 1 mg/kg and 3 mg/kg (every 5 days for 3 doses then every 3 days for 3 doses), co-formulated ZF9-MQ1+ZF3-KRAB at 1 mg/kg and 3 mg/kg (every 5 days for 3 doses then every 3 days for 3 doses), negative control mRNA at 1 mg/kg and 3 mg/kg (every 5 days for 3 doses then every 3 days for 3 doses), intraperitoneal with MYCi975 at 100 mg/kg (once daily for 5 days per week) or intraperitoneal with cisplatin at 1 mg/kg (once every 15 days). All animals were weighed daily and assessed visually. Changes in tumor volume were measured 3 times per week.

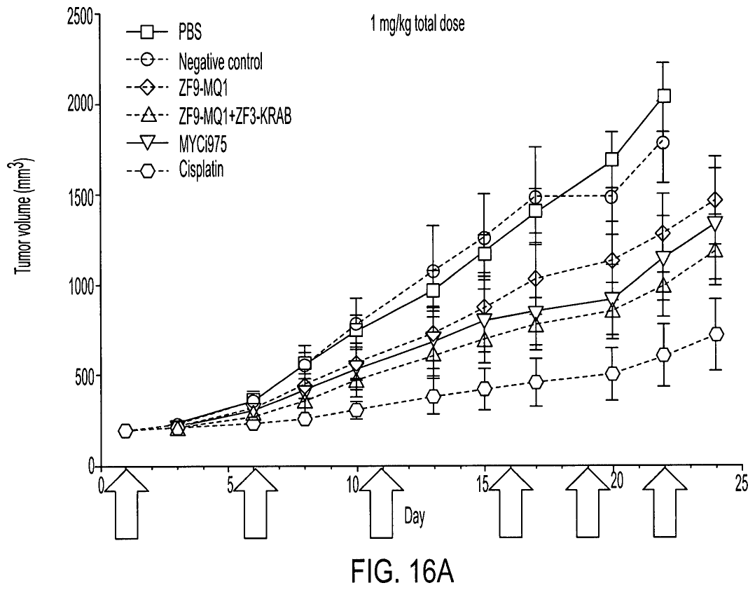

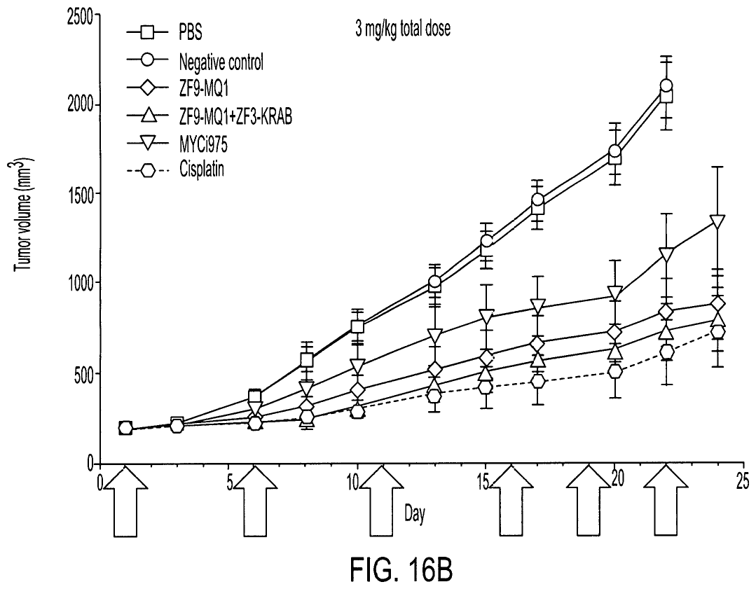

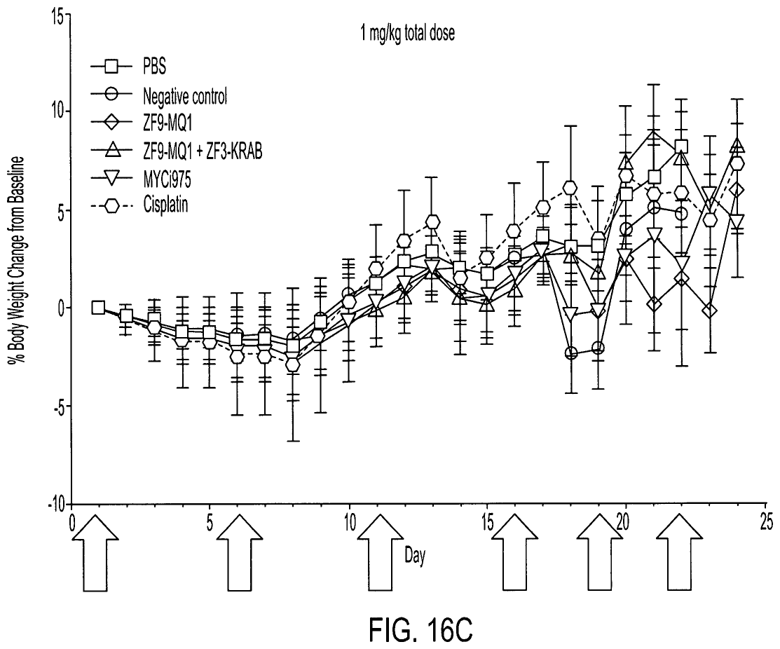

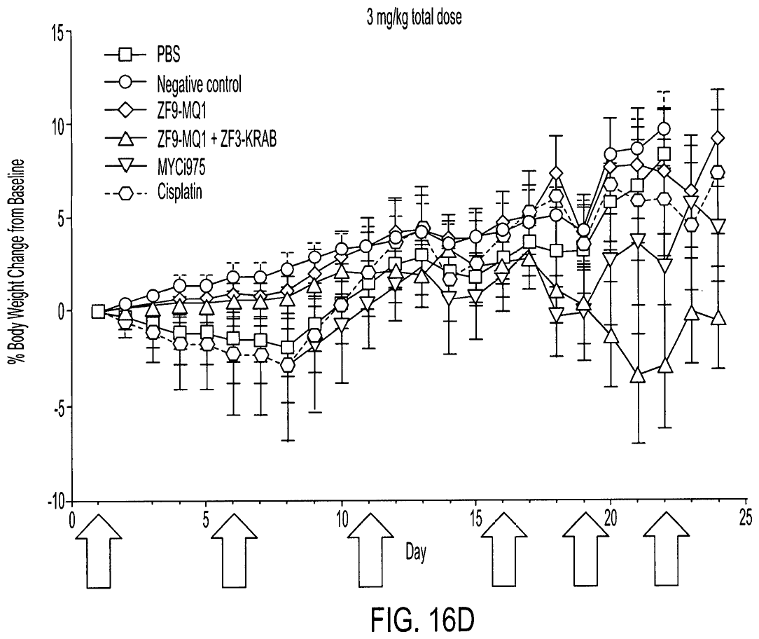

The results show that ZF9-MQ1 individually and the combination of ZF9-MQ1 and ZF3-KRAB at 1 mg/kg were able to reduce tumor growth when compared to negative control treated mice (FIG. 16A). Further, ZF9-MQ1 individually (from day 13 forward) and the combination of co-formulated ZF9-MQ1+ZF3-KRAB (from day 6 forward) at 3 mg/kg were able to significantly reduce tumor growth when compared to negative control treated mice (FIG. 16B). Co-formulated ZF9-MQ1+/ZF3-KRAB was able to reduce tumor growth at a similar or a greater level than cisplatin or the small molecule comparator (MYCi975) at both 1 mg/kg and 3 mg/kg dosage. Co-formulated ZF9-MQ1+/−ZF3-KRAB had minimal effect on overall animal weight compared to both cisplatin and MYCi975 at both 1 mg/kg and 3 mg/kg dosage (FIG. 16C-D).

### Example 16: Combination of Zinc Finger Domains Plus a DNA Methyltransferase Targeted to MYC Insulated Genomic Domain (IGD) Down-Regulates MYC Expression and Reduce Cell Viability in Various Lung Cancer Cell Lines

This example demonstrates that down-regulating Myc1 mRNA expression by targeting MQ1 effector fused to zinc-finger domain to the MYC promoter (ZF9-MQ1) in NSCLC lines (A549, NCI-H2009, NCI-H358HCC95) results in loss of cell viability across numerous lung cancer cell lines.

ZF9-MQ1 designed to bind and target the MYC promoter in lung cancer cell lines was delivered alongside negative controls, which included untreated cells and cells transfected with green-fluorescent protein (GFP). The mRNAs encoding either ZF9-MQ1 or GFP were delivered using SSOP LNP delivery as described in Examples 12 and 14 above. Lung cancer cells were seeded in 96-well plates in growth media (10000 cells/well). LNP formulations (1 ug/ml) were then added to the cells to transfect the mRNA then incubated for 72 hours and 120 hours respectively. Different replicate plates were collected for determining change in viability and mRNA expression.

Viability was measured using the CellTiter-GLO® assay kit from Promega according to manufacturer's protocol. RNA was isolated from three biological replicates, using the Rneasy® Plus 96-well Kit (Qiagen) following the Manufacture's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH reference genes using the ΔΔCt method, the untreated samples were used as calibrators.

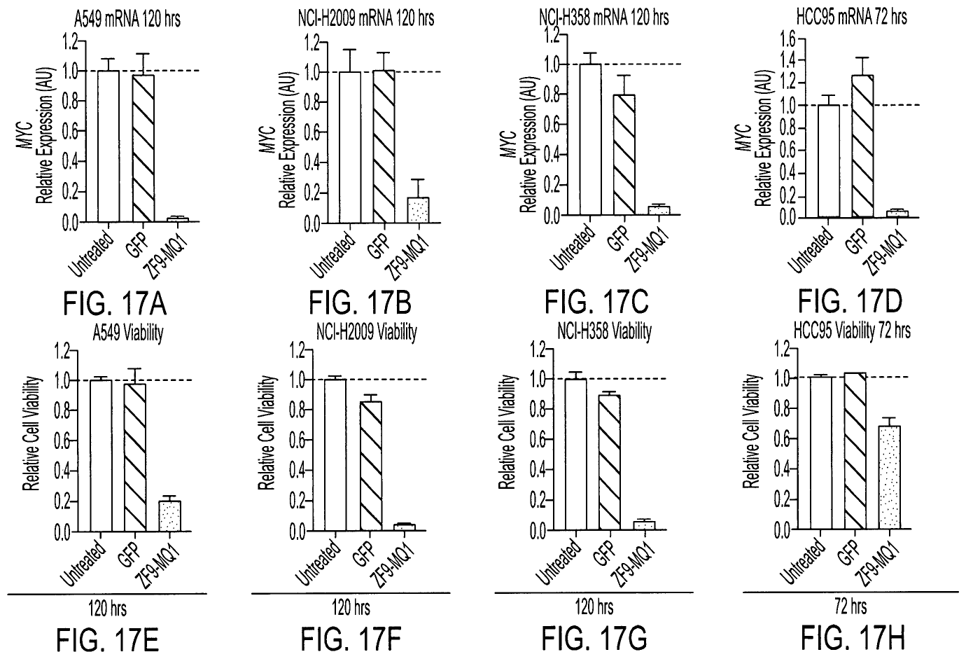

These data showed that ZF9-MQ1 is capable of reducing MYC mRNA levels >80% in four lung cancer cells lines and this coincided with significant loss of lung cancer cell viability across all four cell lines (FIG. 17A-H).

### Example 17: ZF9-MQ1 Induces Cellular Apoptosis in NSCLC Cell Line NCI-H2009

This example demonstrates the effect of ZF9-MQ1 on cellular apoptosis of lung cancer cells.

Viability assays such as Cell Titer GLO (used in Example 16) measures viability by determining the relative number of cells remaining in the well based on levels of ATP. These assays do not distinguish between a loss of cell proliferation and different types of cell death (e.g., necroptosis vs apoptosis).

Fluorescently tagged antibodies to the ANNEXIN V protein and propidium iodide (PI) nuclear stain were utilized to quantify apoptotic cells following transfection with ZF9-MQ1. mRNA encoding the ZF9 zinc finger domain without an effector protein (ZF9-NE) was used a negative control in addition to untreated cells. Lung cell line NCI-H2009 was plated in a 12 well plate in growth media (50,000 cells per well). LNP formulations with mRNA (1 ug/ml) were then added to the cells to transfect the ZF9-MQ1 or ZF9-NE mRNA then incubated for 96 hours. Cells were harvested and stained using the BD Annexin V: FITC apoptosis detection kit (BDB556570) and analyzed by flow cytometry. Cells positive for Annexin V-FITC and PI were categorized as apoptotic.

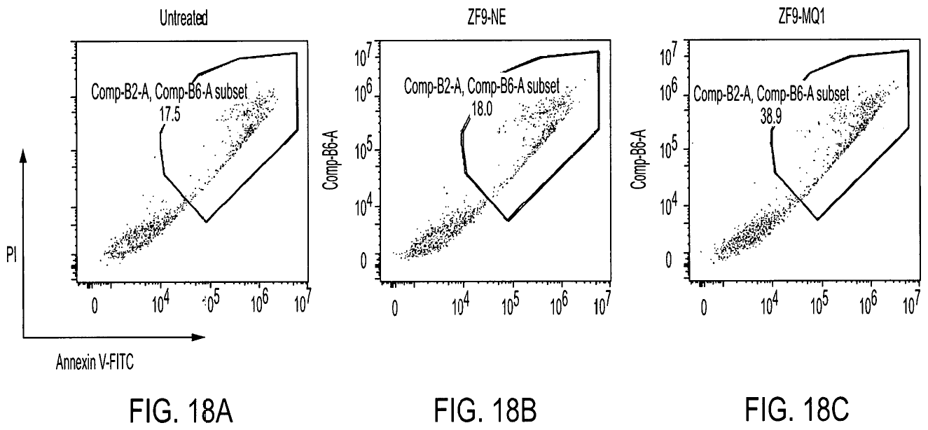

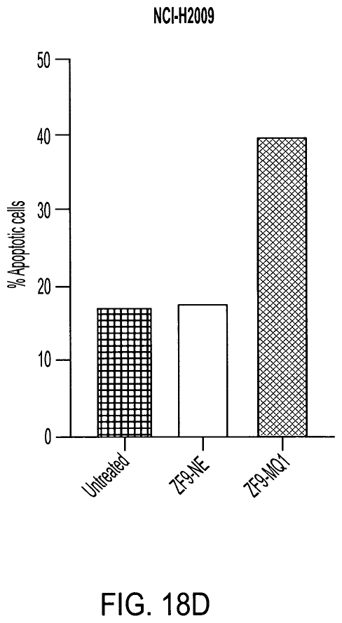

These data showed that after 96 hours in the negative control (cells treated with ZF9-NE and untreated cells) only 18% of the cells were apoptotic (FIG. 18A-B, 18D). In contrast, 40% of the cells were apoptotic in the NCI-H2009 culture treated with ZF9-MQ1 (FIG. 18C-D). This indicates that ZF9-MQ1 could induce cellular apoptosis of lung cancer cells.

### Example 18: Dose Response Curves of Viability and MYC Expression for ZF9-MQ1 in Additional NSCLC Cell Lines

This example demonstrates ZF9-MQ1 down-regulates MYC1 expression and cell viability in NSCLC cell lines in a dose responsive manner.

ZF9-MQ1 designed to bind and target the MYC promoter was dosed at multiple concentration in two NSCLC cell lines, A549 and HCC95. Untreated cells were used as negative controls. The effector mRNAs were delivered using LNP delivery with SSOP. Lung cancer cells were seeded in 96-well plates in growth media (˜10000 cells/well). LNP formulations (starting at 5 ug/ml) were then added to 3 wells each then diluted ˜1:2 in subsequent wells for 10 doses in order to transfect mRNA and incubated for 72 hours. Different replicate plates were collected for determining change in viability and mRNA expression.

Viability was measured using the CellTiter-GLO® assay kit from Promega according to manufacturer's protocol. RNA was isolated from three biological replicates, using the Rneasy® Plus 96-well Kit (Qiagen) following the Manufacturer's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH reference genes using the ΔΔCt method, the untreated and dCas9(no-effector) samples were used as calibrators.

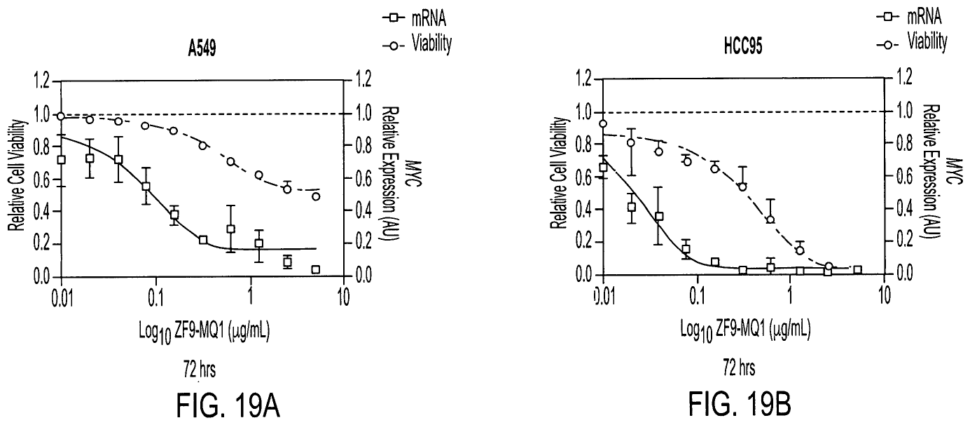

Data showed that ZF9-MQ1 down-regulated MYC with an EC50 of 0.08 ug/ml LNP/mRNA with a ˜25-fold higher EC50 effect on viability (2 ug/ml) in vitro at 72 hours in both A549 and HCC95 (FIG. 19A-B).

### Example 19: ZF9-MQ1 Reduces MYC Protein Levels Over 80% in NSCLC Cell Line NCI-H2009

This example demonstrates alteration of MYC protein level in response to ZF9-MQ1 treatment through an immunoblotting technique.

ZF9-MQ1 designed to bind and target the MYC promoter in lung cancer cell lines was delivered alongside negative controls ZF9-NE and untreated cells. Lung cell line NCI-H2009 was plated in a 12 well plate in growth media (50,000 cells per well). LNP formulations (1 ug/ml) were then added to the cells to transfect the ZF9-MQ1 or ZF9-NE mRNA then incubated for 96 hours. Cells were then lysed in RIPA buffer and protein levels quantified using the Pierce BCA protein assay (23225). Equal amounts of protein were loaded for each sample and separated by size using the NuPAGE™ mini gel system (Thermo Fisher). Protein was then transferred to PVDF membrane using the iBlot™ 2 gel transfer device (Thermo Fisher). Membranes were probed overnight with ABCAM anti-MYC antibody (ab32072). Cell Signaling anti-ACTIN antibody (8H10D10) was used as a loading control. Signal was then visualized and quantified using the LI-COR imaging system using fluorescent secondary antibodies to the MYC and ACTIN antibody species.

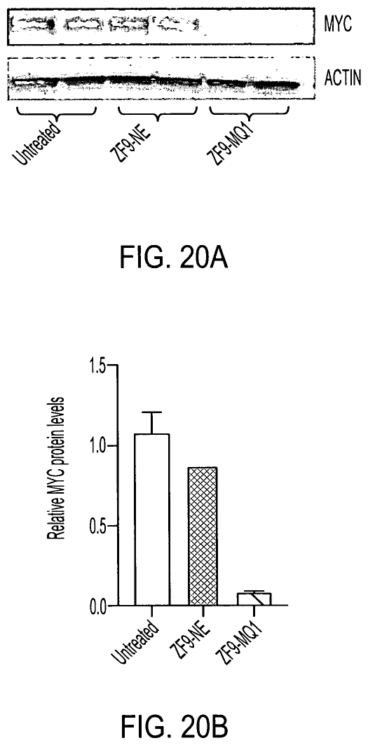

Data showed that ZF9-MQ1 treatment reduces MYC protein levels over 80% 96 hours post-treatment in lung cancer cell lines (FIG. 20A-B), comparable to the reduction in mRNA expression level (Example 17).

### Example 20: In Vivo Efficacy of ZF9-MQ1 in NCI-H2009 Model Grown Subcutaneously in Nude Mice

This example demonstrates that ZF9-MQ1 inhibits the growth of NCI-H2009 tumors established in female nude mice.

Disease was induced in female nude mice by the implantation of NCI-H2009 tumor cells into the left flank. Treatment was initiated when mean tumor volume reached approximately 100-150 mm³. Mice were divided into treatment groups so that mean tumor volume in each group were approximately equal. mRNA was delivered in the MC3 LNP. Mice were injected intravenously with ZF9-MQ1 or a non-coding mRNA in MC3 LNPs at 3 mg/kg or PBS (once every 5 days for 4 doses then once every 3 days for 3 doses; mice were given 7 doses in total). All animals were weighed daily and assessed visually. Tumor sizes were measured 3 times per week.

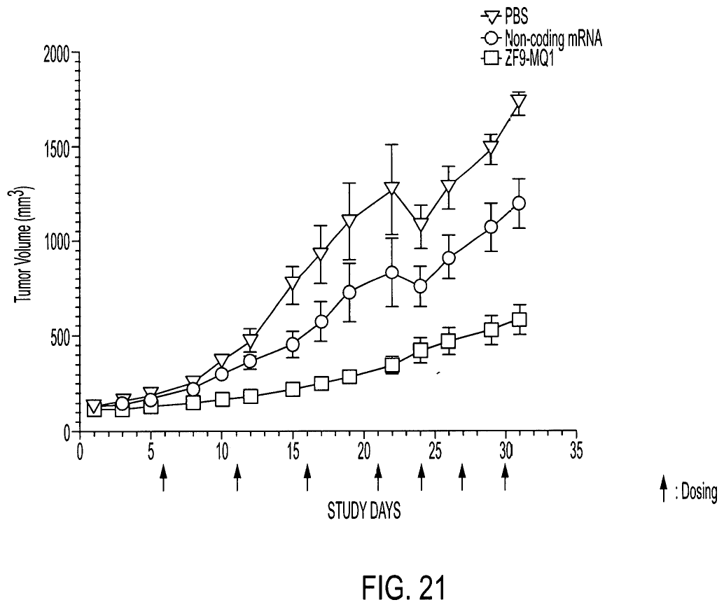

The results showed that ZF9-MQ1 was able to significantly reduce tumor growth (from day 8 forward) when compared to PBS control treated mice (FIG. 21). ZF9-MQ1 had minimal effect on overall animal weight.

### Example 21: Combination of Guide RNAs Plus Transcriptional Repressors (Via dCas9) Targeted to MYC IGD Super-Enhancer Down-Regulate MYC Expression Using SSOP LNP

This example demonstrates MYC mRNA expression is downregulated when the lung specific super-enhancer which regulates MYC expression in A549 cell line is targeted by KRAB effector proteins.

Guide RNAs (Table 13) were designed across the lung super-enhancer region and tested in combination with the KRAB repressor protein conjugated to an enzymatically dead CAS9. The effector mRNA and guide RNAs were co-delivered using LNP delivery with SSOP with mRNA delivery of GFP as a negative control. NCSLC cell line A549 were seeded in 96-well plates in growth media (10000 cells/well). LNP formulations (2.5 ug/ml) were then added to the cells to transfect the effector mRNA/guide RNA then incubated for 72 hours. RNA was isolated from three biological replicates, using the Rneasy® Plus 96-well Kit (Qiagen) following the Manufacture's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH reference genes using the ΔΔCt method, the untreated samples were used as calibrators.

TABLE 13
Guides

| Guide Name | Target Sequence | Genomic Coordinates |
|---|---|---|
| GD-29833 | TGCCACTTCCCCACTAACCC | GRCh37: chr8:129188878-129188900 |
| | (SEQ ID NO: 96) | |
| GD-29834 | GGCCACACAAGGAAGCTGCA | GRCh37: chr8:129188958-129188980 |
| | (SEQ ID NO: 97) | |
| GD-29835 | CCACACAAGGAAGCTGCAGG | GRCh37: chr8:129188960-129188982 |
| | (SEQ ID NO: 98) | |
| GD-29836 | TGATTGGAATGCAACCCGAA | GRCh37: chr8:129189067-129189089 |
| | (SEQ ID NO: 99) | |
| GD-29837 | TTTTGCCCTTGCTACCCCAA | GRCh37: chr8:129189457-129189479 |
| | (SEQ ID NO: 100) | |
| GD-29838 | AGCTGATGGTATCCACTAGG | GRCh37: chr8:129189554-129189576 |
| | (SEQ ID NO: 101) | |
| GD-29839 | CACATCCAAGAATGTAGTGG | GRCh37: chr8:129189679-129189701 |
| | (SEQ ID NO: 102) | |
| GD-29840 | GATACAGCCACAAAGCTCAC | GRCh37: chr8:129209511-129209533 |
| | (SEQ ID NO: 103) | |
| GD-29841 | ATTACATAACAGAATCCAGG | GRCh37: chr8:129209643-129209665 |
| | (SEQ ID NO: 104) | |
| GD-29842 | CCCTTGACTGTGCTGCCACC | GRCh37: chr8:129209658-129209680 |
| | (SEQ ID NO: 105) | |
| GD-29843 | CAGACGAGGAACCTGAACCC | GRCh37: chr8:129209856-129209878 |
| | (SEQ ID NO: 106) | |
| GD-29844 | AGAATCCCTTGGGGTAGCAA | GRCh37: chr8:129189452-129189474 |
| | (SEQ ID NO: 107) | |
| GD-29914 | CAGCACTCTCGCTGACCGCA | GRCh37: chr8:129189190-129189212 |
| | (SEQ ID NO: 108) | |
| GD-29915 | GTTGAGTCATGTGTACTCTG | GRCh37: chr8:129189274-129189296 |
| | (SEQ ID NO: 109) | |
| GD-29916 | AGGAACAGGATGTTACAACT | GRCh37: chr8:129189421-129189443 |
| | (SEQ ID NO: 110) | |
| GD-28662 | GGGGCCACTAGGGACAGGAT | GRCh37: chr19:55627120-55627139 |
| | (SEQ ID NO: 111) | |

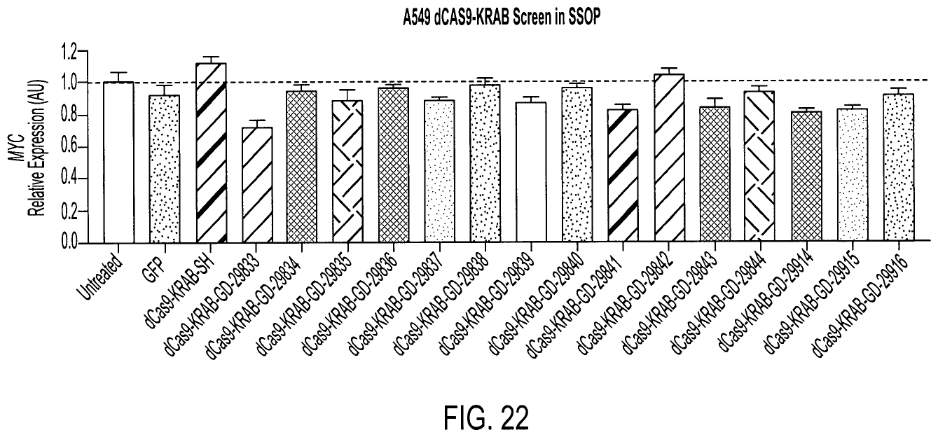

These data showed that guide RNA GD-29833 and 29914 could downregulate MYC mRNA levels when delivered with a dCAS9-KRAB effector mRNA, highlighting the ability to decrease oncogenic MYC using this distal regulatory element (FIG. 22).

### Example 22: Combination of Guide RNAs Plus Transcriptional Repressors (dCas9) Targeted to MYC IGD Super-Enhancer Down-Regulate MYC Expression Using MC3 LNP

This example describes down-regulating MYC mRNA expression by targeting KRAB effector proteins to the lung specific super-enhancer which regulates MYC expression utilizing an alternative lipid MC3 (vs. SSOP in Example 21).

GuideRNAs (Table 13) were designed across the lung super-enhancer region and tested in combination with the KRAB repressor protein conjugated to an enzymatically dead CAS9. The effector mRNA and guide RNAs were co-delivered using LNP delivery with MC3 with mRNA delivery of GFP as a negative control. NCSLC cell line A549 were seeded in 96-well plates in growth media (10000 cells/well). LNP formulations (2.5 ug/ml) were then added to the cells to transfect the effector mRNA/guide RNA then incubated for 72 hours. RNA was isolated from three biological replicates, using the Rneasy® Plus 96-well Kit (Qiagen) following the Manufacture's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH reference genes using the ΔΔCt method, the untreated samples were used as calibrators.

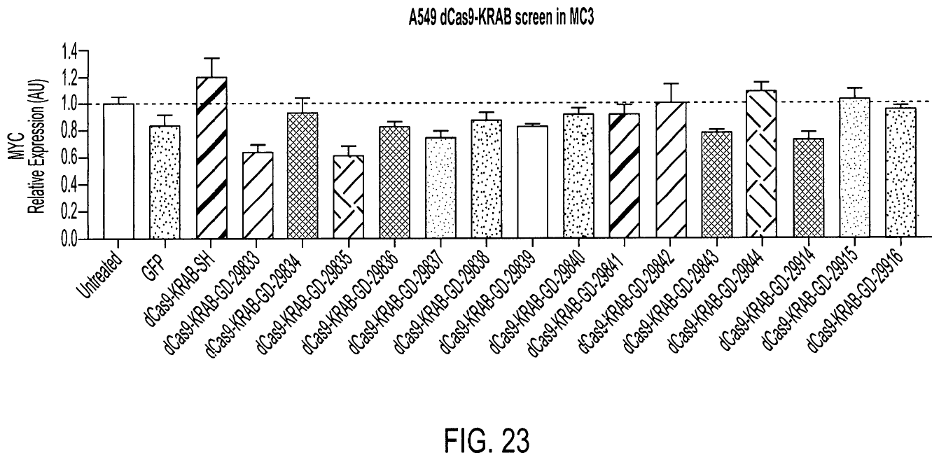

These data showed that guide RNA GD-29833 and 29914 could downregulate MYC mRNA levels when delivered with a dCAS9-KRAB effector mRNA, highlighting the ability to decrease oncogenic MYC using this distal regulatory element (FIG. 23). This effect was seen with both SSOP (Example 21) and MC3 lipid transfection (Example 22).

### Example 23: Combination of Guide RNAs Plus Transcriptional Repressors (dCas9) Targeted to MYC IGD Super-Enhancer Down-Regulate MYC Expression in NSCLC

This example describes down-regulating MYC mRNA expression by targeting various transcriptional effector proteins (EZH2, EZH2-KRAB, or MQ1) to the lung specific super-enhancer which regulates MYC expression.

Guide RNAs (GD-29833 and GD-29914) targeted to the MYC super-enhancer were tested in combination with repressor proteins including the histone methyltransferase EZH2 (alone or in conjunction with KRAB) and the DNA methyltransferase MQ1 conjugated to an enzymatically dead CAS9. The effector mRNA and guide RNAs were co-delivered using LNP delivery with SSOP with mRNA delivery of GFP as a negative control. NCSLC cell lines A549 or NCI-H2009 were seeded in 96-well plates in growth media (10000 cells/well). LNP formulations (2.5 ug/ml) were then added to the cells to transfect the effector mRNA/guide RNA then incubated for 72 hours. RNA was isolated from three biological replicates, using the Rneasy® Plus 96-well Kit (Qiagen) following the Manufacture's protocol. RNA samples were retrotranscribed to cDNA using LunaScript® RT SuperMix Kit (NEB) and analyzed by quantitative PCR (qPCR) (in technical triplicates) using a MYC specific TaqMan′ primer/probe set assay with the TaqMan™ Fast Advanced Master Mix (Thermo Scientific). MYC expression was quantified relative to the expression of GAPDH reference genes using the ΔΔCt method, the untreated samples were used as calibrators.

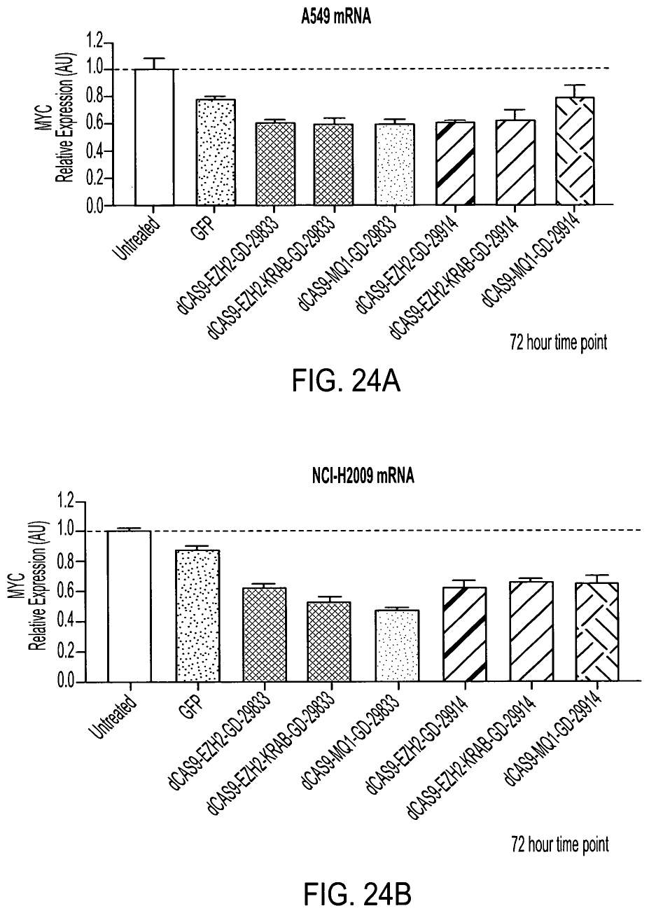

These data showed that guide RNA GD-29833 and 29914 could effectively downregulate MYC mRNA levels when delivered with all 3 effector proteins (EZH2, EZH2-KRAB, and MQ1) tested in 2 different NSCLC cell lines (A549 and NCI-H2009) (FIG. 24A-B).

### Example 24: Combination of Guide RNAs Plus Transcriptional Repressors (dCas9) Targeted to MYC IGD Super-Enhancer Further Down-Regulate MYC Expression in NSCLC at 120 Hours

This example demonstrates an increased decrease in MYC mRNA expression observed at 120 hours following transfection with super-enhancer targeted guides with KRAB or MQ1 effector proteins in lung cancer cell lines (A549 and NCI-H2009).

Guide RNAs targeted to the MYC super-enhancer were tested in combination with the KRAB repressor protein or the MQ1 DNA methyltransferase. The effector mRNA and guide RNAs were co-delivered using LNP delivery with SSOP with mRNA delivery of GFP as a negative control. NCSLC cell lines A549 or NCI-H2009 were seeded in 12 well plates in growth media (˜50000 cells/well). LNP formulations (2.5 ug/ml) were then added to the cells to transfect the effector mRNA/guide RNA then incubated for 120 hours. RNA was isolated from three biological replicates, using the Rneasy® Plus 96-well Kit (Qiagen) following the Manufacture's protocol. RNA samples were retrotranscribed to cDNA using LunaScript® RT SuperMix Kit (NEB) and analyzed by quantitative PCR (qPCR) (in technical triplicates) using a MYC specific TaqMan′ primer/probe set assay with the TaqMan′ Fast Advanced Master Mix (Thermo Scientific). MYC expression was quantified relative to the expression of GAPDH reference genes using the ΔΔCt method, the untreated samples were used as calibrators.

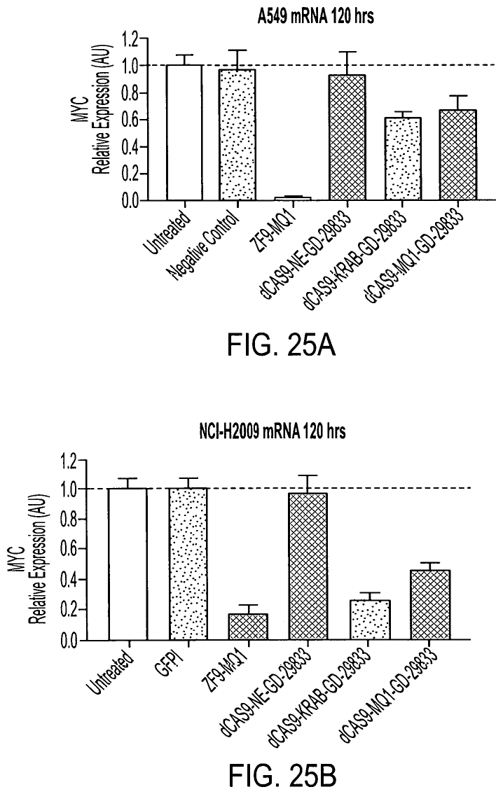

These data showed that guide RNA GD-29833 and 29914 delivered with KRAB or MQ1 could significantly downregulate MYC mRNA levels in 2 NSCLC cell lines at 120 hours (A549 and NCI-H2009) (FIG. 25A-B). Furthermore, observed downregulation is comparable to the downregulation observed by ZF9-MQ1 treatment in NCI-H2009 (FIG. 25B).

### Example 25: Directing dCAS9-MQ1 to the MYC Super-Enhancer Results in Increased DNA Methylation at the Super-Enhancer Target Site and the MYC Promoter Region

This example demonstrates that dCas9-MQ1 can be directed to the MYC super-enhancer resulting in methylation of the target site and the MYC promoter region.

The CRISPR-dCas9 system was modified by tethering it to epigenetic repressor: MQ1. These molecules modulate transcriptional repression methylating the CpG nucleotides of the DNA. The effector mRNA and super-enhancer guide RNAs (29833 and 29914) were co-delivered using LNP delivery with SSOP with mRNA delivery of GFP or a dCAS9-no effector construct (dCas9-NE) as negative controls. NCSLC cell line NCI-H2009 was seeded in a 6 well plates in growth media (˜100000 cells/well). LNP formulations (2.5 ug/ml) were then added to the cells to transfect the effector mRNA/guide RNA then incubated for 72 hours. DNA was isolated using Lucigen QuickExtract™ DNA extraction kit and methylated regions were determined using targeted bisulfite sequencing of the promoter and super-enhancer regions.

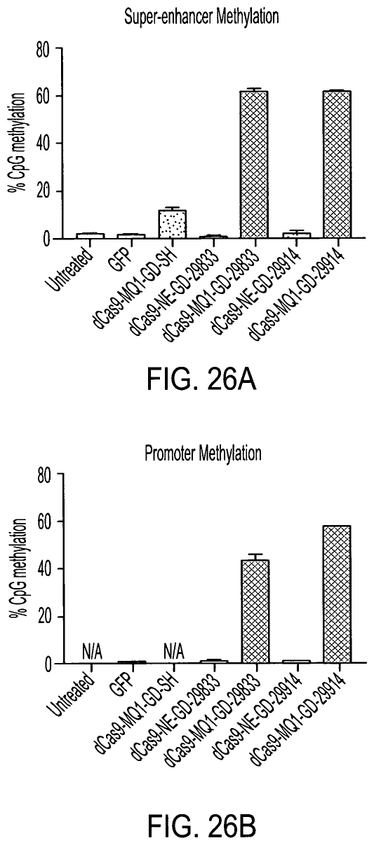

These studies demonstrated that dCas9-MQ1 increased target site methylation in NSCLC by 60% and also directed methylation to the distal promoter region (increased to about 50%), (FIG. 26A-B).

### Example 26: Combination of Guide RNAs Plus Transcriptional Repressors (Via “Dead” CAS9) Targeted to MYC IGD Super-Enhancer Reduce MYC Protein Levels in NSCLC Cell Line NCI-H2009

This example demonstrates alteration in the levels of MYC protein by targeting guide RNAs to the MYC super-enhancer in NSCLC cells through an immunoblotting technique.

GD-29833 designed to bind and target the MYC super-enhancer in lung cancer cell lines was co-delivered with dCAS9-KRAB or dCAS9-MQ1 effector mRNA. Lung cell line NCI-H2009 was plated in a 12 well plate in growth media (50,000 cells per well). LNP formulations (1 ug/ml) were then added to the cells to transfect guide and effector mRNAs then incubated for 96 hours. A dCAS9-no effector (dCAS9-NE) construct was used as a negative control. Cells were then lysed in RIPA buffer and protein levels quantified using the Pierce BCA protein assay (23225). Equal amounts of protein were loaded for each sample and separated by size using the NuPAGE™ mini gel system(Thermo Fisher). Protein was then transferred to PVDF membrane using the Invitrogen iBlot™ 2 gel transfer device (Thermo Fisher). Membranes were probed overnight with ABCAM anti-MYC antibody (ab32072). Cell Signaling anti-ACTIN antibody (8H10D10) was used as a loading control. Signal was then visualized and quantified using the LI-COR imaging system using fluorescent secondary antibodies to the MYC and ACTIN antibody species.

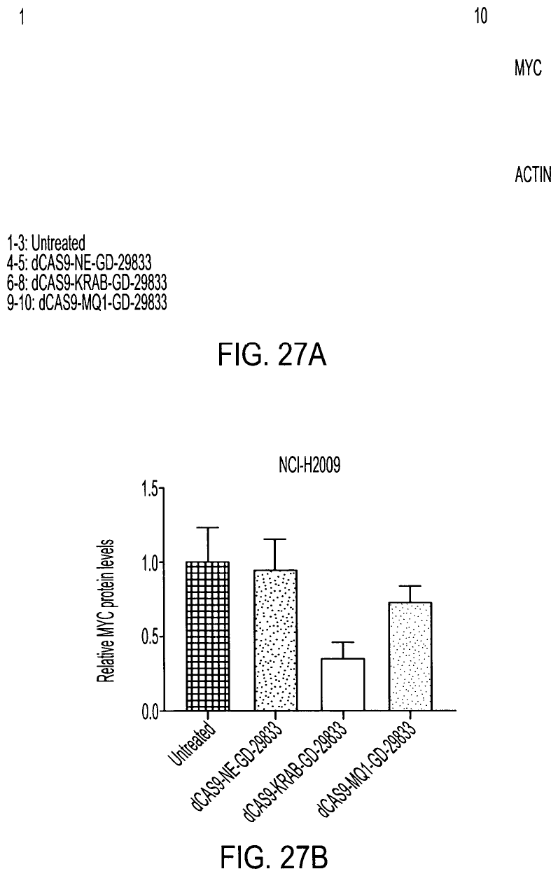

These data showed that directing guides to the MYC lung super-enhancer with transcriptional repressors reduces MYC protein levels up to 50% at 96 hours in NCI-H2009 lung cancer cell lines (FIG. 27A-B), comparable to the reduction in mRNA expression level (Example 16).

### Example 27: ZF9-MQ1 Protein Presence in Whole Cell Lysate Correlates with Down Regulation of MYC Protein in Hep3B Cell Line

The example describes determination of the change ZF9-MQ1 and MYC protein expression level over a time course in Hep 3B cells after treatment with ZF9-MQ1.

ZF9-MQ1 and MYC protein expression levels were assessed (western blot) at 6, 16 and 48 hours after ZF9-MQ1 treatment, with LNPs being removed and media replaced 24 hours after treatment initiation. Protein was extracted using RIPA buffer and total protein was quantitated using BCA assay (Thermo Fisher). Total protein was run on NuPAGE™ Bis-Tris Gels (Thermo Fisher), MOPS running buffer and transferred using the iBlot™ 2 Gel Transfer Device (Thermo Fisher).

MYC western: β-actin antibody was stained with a secondary antibody tagged with fluorophore emitting at 594 nm wavelength and MYC antibody (Abcam) was stained with secondary antibody tagged with fluorophore emitting at a wavelength of 488 nm. Odyssey® CLx Imaging System (LI-COR) using near-infrared (NIR) fluorescence was used to capture the protein images that were used for quantitation via the LI-COR software. Area under the curve (AUC) for each MYC and ACTIN band had a background area subtracted then all were normalized to negative control for each timepoint.

ZF9-MQ1 (controller tagged with hemagglutinin [HA] epitope) western: β-actin antibody was stained with a secondary antibody tagged with fluorophore emitting at a wavelength of 594 nm and HA antibody was stained with secondary antibody tagged with fluorophore emitting at a wavelength of 488 nm. Odyssey® CLx Imaging System (LI-COR) using MR fluorescence was used to capture the protein images that were used for quantitation via the LI-COR software. AUC for each HA and ACTIN band had a background area subtracted then all were normalized to negative control for each timepoint.

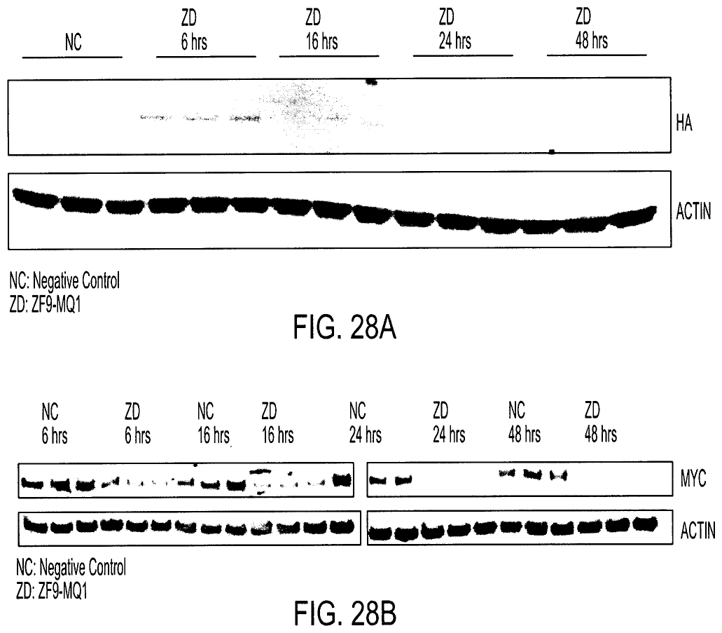

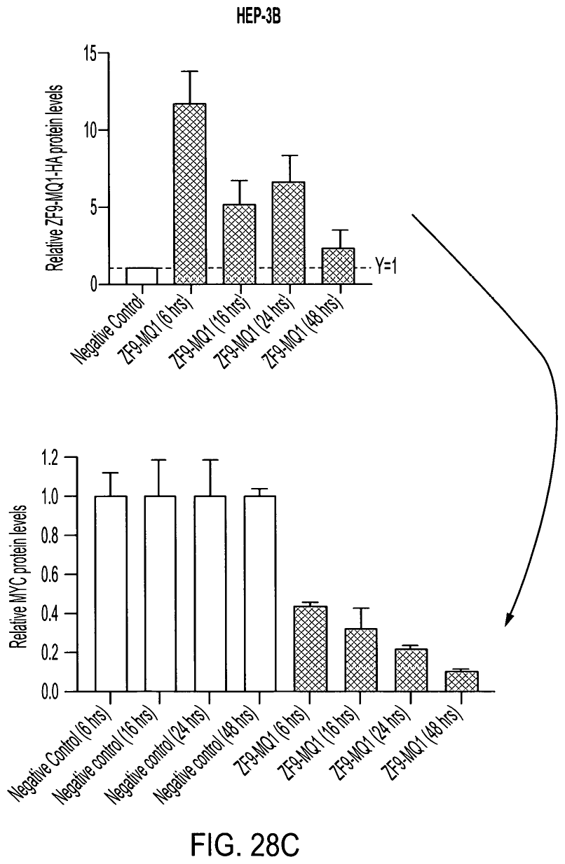

Data shows both ZF9-MQ1 protein presence and MYC protein expression level decrease in the whole cell lysate after treating the cells with ZF9-MQ1 (FIGS. 28A-B) and the ZF9-MQ1 protein presence in whole cell lysate correlates with down regulation of MYC protein (FIG. 28C).

### Example 28: Duration of Reduction in MYC Expression and Methylation Status

The experiment assesses the durability of reduction of MYC expression after treatment with ZF9-MQ1. Additionally, this experiment demonstrates and assesses the correlation of MYC expression with increased DNA methylation at the target locus.

The SKHEP-1 cell line which demonstrated minimal changes to viability but 40-50% MYC down-regulation (Example 4) was utilized to assess the durability of the response. SK-HEP-1 were transfected with LNP/ZF9-MQ1 or ZF-no effector (negative control) then replaced with new media after 24 hours of treatment. At designated timepoints cells are collected for extraction of mRNA and genomic DNA (Qiagen RNA/DNA kit). To assess MYC mRNA, whole cell RNA was processed to make complementary DNA (cDNA) (using a poly-A primer) that was then used for reverse transcription polymerase chain reaction (RT-PCR) analysis using TaqMan™ probes (Thermo Fisher) specific for human MYC mRNA transcripts. To assess methylation status at targeted loci, targeted bisulfite genomic sequencing was used to measure 5-methylcytosine at a single DNA base pair resolution.

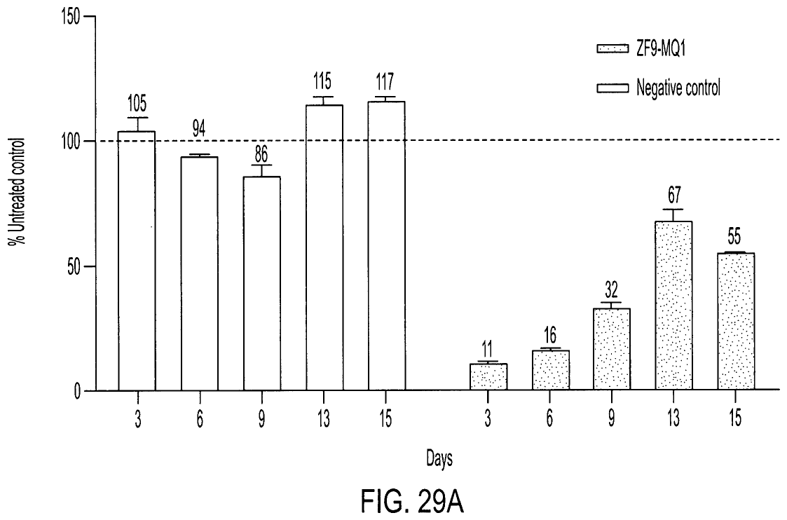

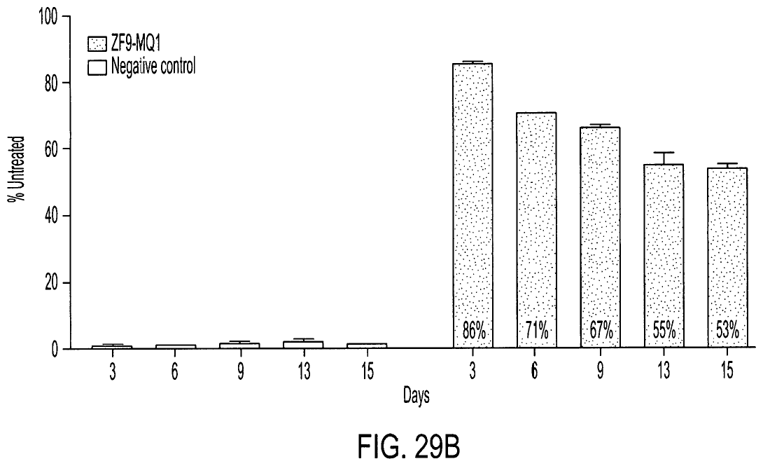

At day 3, MYC mRNA was decreased by 89% as compared to a negative control (ZF-no effector) and untreated cells (not shown). Down regulation of mRNA expression slowly increased with a 45% down-regulation in MYC transcript being maintained on Day 15 (FIG. 29A). In addition, expression of ZF9-MQ1 directed de novo CpG methylation to the MYC promoter region. MYC transcriptional changes correlated with the percentage of methylation out to day 15 (FIG. 29B).

### Example 29: C-MYC Expression and Cell Viability with Bi-Cistronic ZF9-MQ1_ ZF3-KRAB on Primary Hepatocytes

The example evaluates the effect of bi-cistronic ZF9-MQ1_ZF3-KRAB on MYC mRNA and viability in primary hepatocytes.

Cryopreserved primary human hepatocytes (Lonza) were thawed and added to prewarmed thawing media and plated from 24 hours. The cells were resuspended in plating media and counted to prepare at specified concentration (106 cells/mL). Fifty (50) uL of cell solution were then added to 96-well plates (with additional 50 uL of the plating media) for 100 uL total volume and incubated overnight. LNP formulated mRNA (GFP, ZF-NE, ZF9-MQ1, ZF3-KRAB, ZF9-MQ1+ZF3-KRAB, or bi-cistronic ZF9-MQ1_ZF3-KRAB) were added to the cells in 100 μL of additional media in 0.6 μg/ml, 1.25 μg/ml, and 2.5 μg/ml concentration. Cells were incubated for 72 hours, with maintenance media replacements starting at 6 hours and daily thereafter. MYC mRNA expression levels (RT-PCR) and cell viability)(CellTiter-GLO®) were assessed 72 hours after treatment.

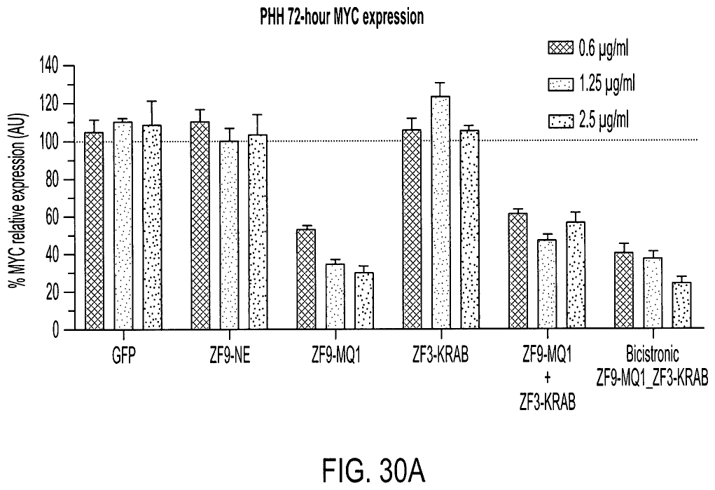

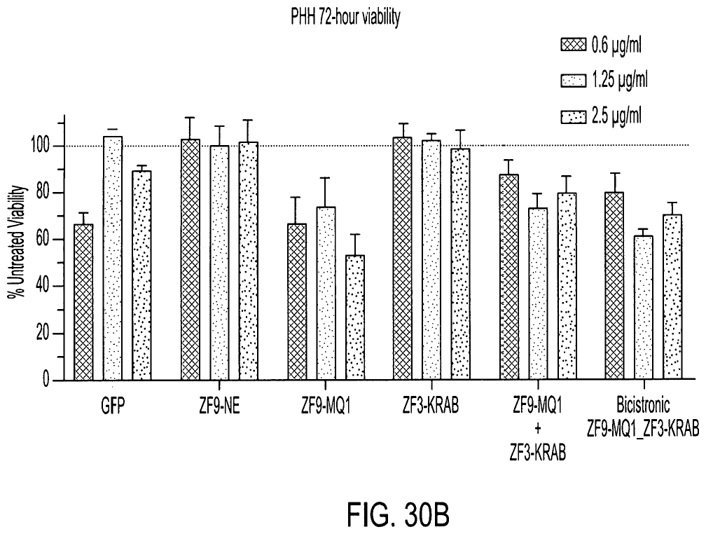

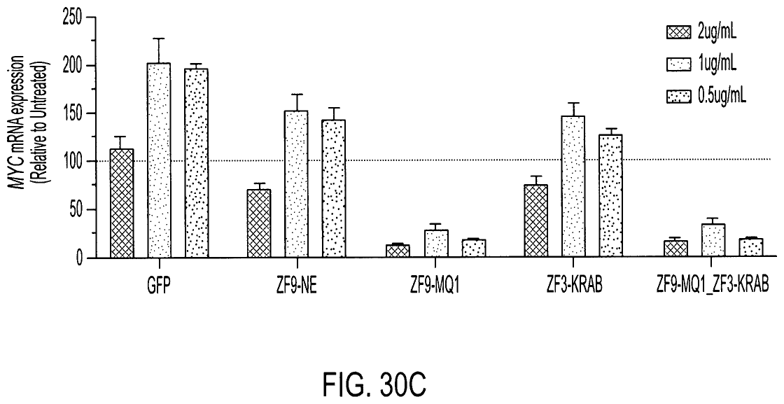

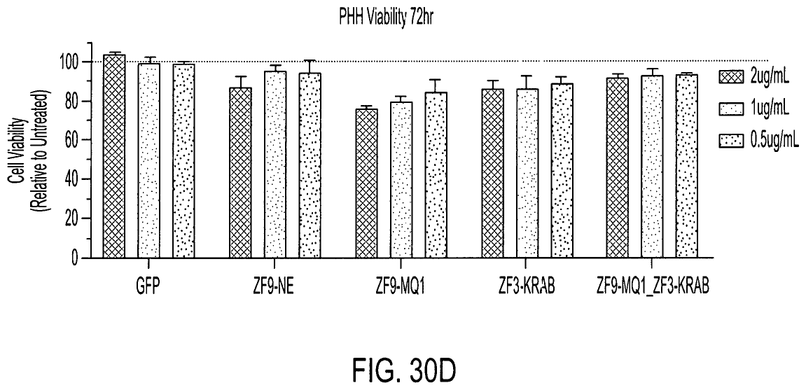

Primary hepatocytes treated with ZF9-MQ1, ZF9-MQ1+ZF3-KRAB, or bi-cistronic ZF9-MQ1_ZF3-KRAB showed a decrease of MYC mRNA expression when compared to GFP, ZF-NE or ZF3-KRAB (FIG. 30A). Overall, the treatment showed minimal effect on viability demonstrating that the decrease in MYC expression is less consequential to normal cells when compared to HCC cell lines (FIG. 30B).

In another experiment, cryopreserved PHH were thawed into prewarmed Hepatocyte Thawing Medium and spun for 8 minutes at 100 g at room temperature. Cell pellet was resuspended in Hepatocyte Plating Medium. Cells were then counted, and their baseline viability was measured. Cell dilution was prepared, and 50,000 cells were plated into duplicate 96-well plates. Cells were incubated overnight. Plating media was completely removed the following day and prewarmed Hepatocyte Culture Medium was added. Cells were treated with bi-cistronic ZF9-MQ1 ZF3-KRAB, ZF9-MQ1, ZF3-KRAB, ZF9-NE or control GFP mRNA at 2.0, 1.0, or 0.5 μg/mL in triplicate and incubated for 6 hours. Treatment media was then removed and replaced with 200 μL fresh Hepatocyte Culture Medium. The cells were incubated for another 66 hours post transfection (72 hours total). Following treatment one plate was lysed with the CellTiter-GLO® reagent with luminescence quantified using the Glo Max Discovery Plate Reader. Media was removed from the second plate of cells by aspiration and cells were lysed with RLT Plus Lysis buffer. mRNA extraction was performed using the Rneasy® Plus 96 Kit according to manufacturer's instructions. After extraction mRNA was converted to cDNA using LunaScript® RT SuperMix Kit (NEB). cDNA was analyzed through ΔΔCT qPCR with a MYC (target) and GAPDH (reference) probe.

PHH treated with bi-cistronic ZF9-MQ1_ZF3-KRAB showed a decrease of MYC mRNA when compared to controls (FIG. 30C). ZF9-MQ1 (all doses) and ZF3-KRAB (2 μg/mL) downregulated MYC mRNA as well with minimal effect on cell viability (FIG. 30D). ZF9-NE did not affect MYC mRNA or viability (FIG. 30C-D). Overall, bi-cistronic ZF9-MQ1_ZF3-KRAB treatment showed minimal effect on viability demonstrating that the decrease in MYC expression in normal cells did not impact cell viability (FIG. 30A-D).

### Example 30: In Vivo Efficacy of ZF9-MQ1+ZF3-KRAB in NCI-H2009 Model Grown Subcutaneously in Nude Mice

This example demonstrates that ZF9-MQ1+ZF3-KRAB inhibits the growth of NCI-H2009 tumors established in female nude mice.

Disease was induced in female nude mice by the implantation of NCI-H2009 tumor cells into the left flank. Treatment was initiated when mean tumor volume reached approximately 100-150 mm³. Mice were divided into treatment groups so that mean tumor volume in each group were approximately equal. mRNA was delivered in the MC3 LNP. Mice were injected intravenously with ZF9-MQ1+ZF3-KRAB at 3 mg/kg at every 5 days, or with an or a non-coding mRNA in MC3 LNPs at 3 mg/kg at every 5 days or with Cisplatin at 1 mg/kg IP every 15 days, or with PBS every 5 days.

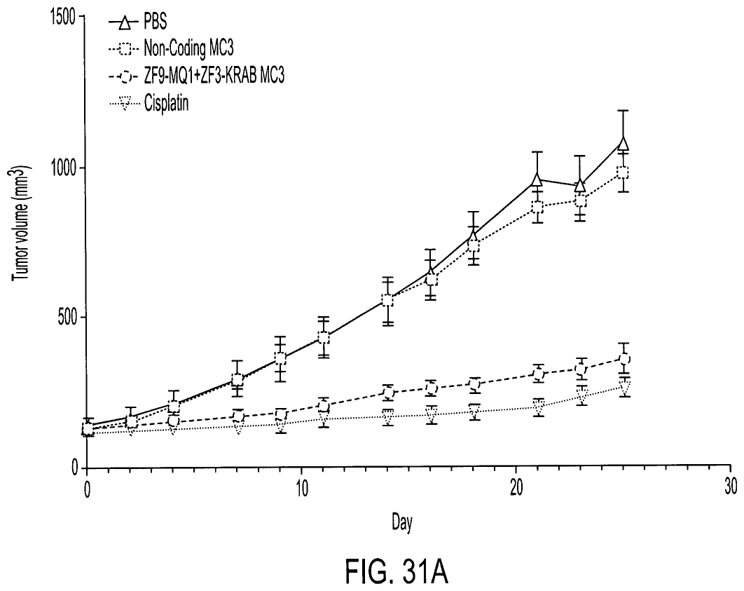

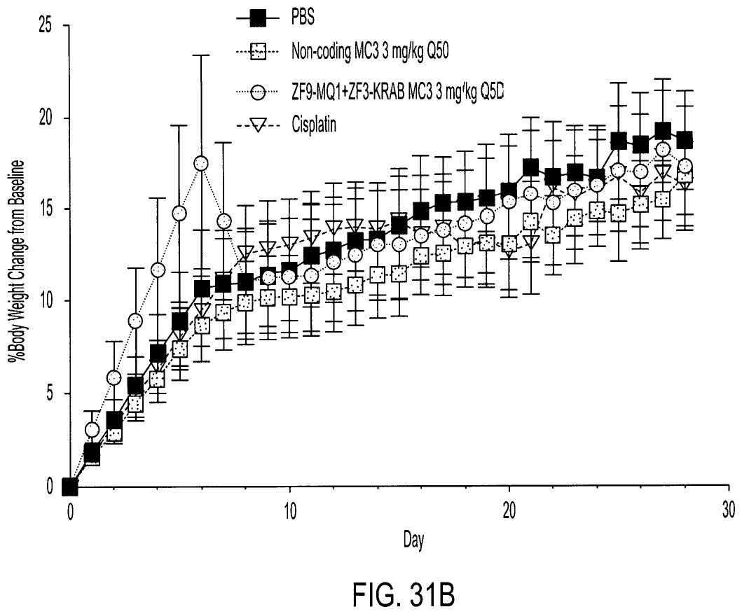

The results showed that treatment with ZF9-MQ1+ZF3-KRAB showed a statistically significant reduction in tumor size following three administrations, resulting in a 63% lower tumor volume at Day 25 compared to control (FIG. 31A), with no significant effect on the body weight of treated mice (FIG. 31B). In this study, ZF9-MQ1+ZF3-KRAB treatment was associated with an equivalent effect on tumor volume to treatment with cisplatin (FIG. 31A).

### Example 31: In Vivo Efficacy of ZF9-MQ1+ZF3-KRAB Co-Formulation in Hep 3B Model Grown Orthotopically in Fox Chase CB17 SCID Mice

This example demonstrates that the long-term anti-tumor efficacy and durability of ZF9-MQ1+ZF3-KRAB co-formulation following dosing, in the orthotopic Hep3B-luc model in female Fox Chase CB17 SCID mice.

Hep-3B-luc cells were injected in the upper left lobe of the liver in SCID mice. The mean ventral view whole body tumor-associated bioluminescence (TABL) for each group at randomization was ˜2.8×10⁹ p/s. Mice were randomly allocated to four groups of 12 mice each for treatment with PBS, ZF9-MQ1+ZF3-KRAB (higher dose, i.e., 6 mg/kg), ZF9-MQ1+ZF3-KRAB (mid dose, i.e. 3 mg/kg), ZF9-MQ1+ZF3-KRAB (low dose, i.e. 3 mg/kg), and one group of 6 mice for treatment with sorafenib on day 7 of the post-implantation of the cells. Treatment started on day 8 post-implantation of tumor cells (marked as day 1 of dosing on graph). Mice were treated intravenously with PBS (every 5 days for 4 doses, then every 3 days for 2 doses), LNP (MC3) ZF9-MQ1+ZF3-KRAB (1.5 mg/kg every 5 days), intravenously with LNP (MC3) ZF9-MQ1+ZF3-KRAB (3 mg/kg every 5 days), intravenously with LNP (MC3) ZF9-MQ1+ZF3-KRAB (6 mg/kg every 5 days), and orally with sorafenib (50 mg/kg daily). All animals were weighed daily and assessed visually. Tumor size were measured by bioluminescence 2 times per week.

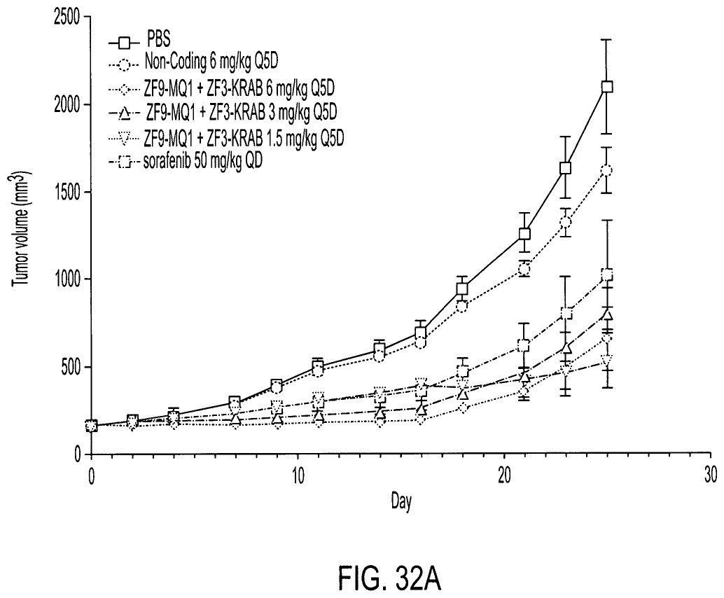

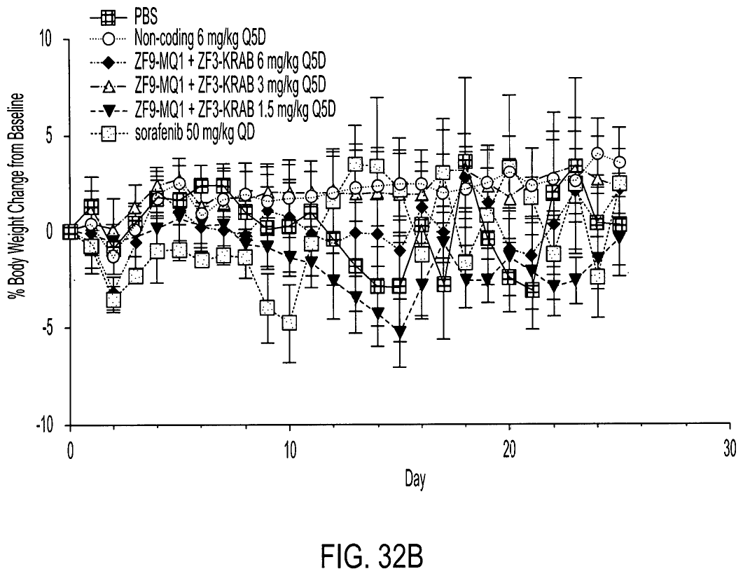

The results showed that treatment with ZF9-MQ1+ZF3-KRAB was associated with significant inhibition in tumor size following two administrations. Treatment with 1.5 mg/kg dose resulted in about 63% inhibition of tumor growth by Day 23 compared to negative control, treatment with 3 mg/kg resulted in about 54% inhibition of tumor growth by Day 23 compared to negative control (FIG. 32A). Similarly, treatment with 6 mg/kg dose of ZF9-MQ1+ZF3-KRAB was associated with a statistically significant reduction in tumor size following two administrations, resulting in 63% lower tumor volume at Day 23 compared to negative control (FIG. 32A). Treatment with ZF9-MQ1+ZF3-KRAB at 3 mg/kg was equivalent to treatment with sorafenib (FIG. 32A). Mice treated with ZF9-MQ1+ZF3-KRAB did not experience a significant decrease in body weight (FIG. 32B). Mice treated with sorafenib experienced an initial drop in body weight with a later gain in overall body weight potentially due to an increase in tumor mass (FIG. 32B). These data suggested that treatment with ZF9-MQ1+ZF3-KRAB was well-tolerated in this study.

### Example 32: Bi-Cistronic mRNA Encoding ZF9-MQ1 and ZF3-KRAB Reduces MYC Expression and Cell Viability

This example compares the efficacy of the bi-cistronic construct ZF9-MQ1_ ZF3-KRAB to single constructs ZF3-KRAB and ZF9-MQ1, and co-formulation of ZF3-KRAB and ZF9-MQ1. These constructs are delivered to hepatocellular carcinoma cells via mRNA encapsulated in lipid nanoparticles (LNPs).

ZF9-MQ1, ZF3-KRAB, bi-cistronic ZF9-MQ1_ZF3-KRAB, and co-formulated ZF9-MQ1 and ZF3-KRAB constructs were prepared by encapsulating the respective mRNAs in LNPs. Hep 3B cells were transfected by seeding 10,000 cells per well in a 96 well plate and further treated with 0.6 μg/mL and 2 μg/mL mRNA/LNPs.

MYC mRNA and cell viability were analyzed 48 hours post transfection. Viability was measured using CellTiter-GLO® assay kit from Promega according to manufacturer's protocol. RNA was isolated from three biological replicates, using the RNeasy® Plus 96-well Kit (Qiagen) following the manufacturer's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with TaqMan™ Fast Advanced Master Mix (Thermo Scientific). MYC expression was quantified relative to the expression of GAPDH genes (using TaqMan™ primer/probes) using the ΔΔCt method. The untreated cells were used to normalize expression of MYC.

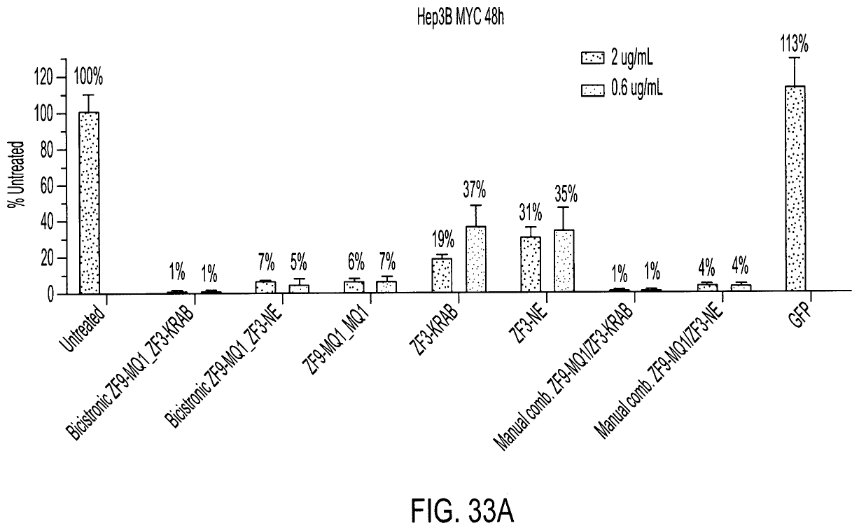

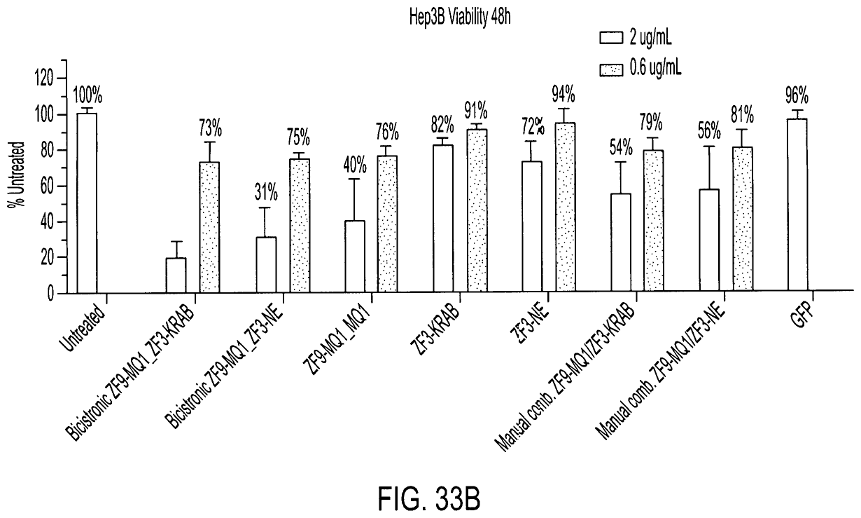

These data showed that the bi-cistronic construct ZF9-MQ1_ ZF3-KRAB downregulated MYC mRNA and cell viability in Hep 3B cells to a greater extent than the single constructs (ZF3-KRAB or ZF9-MQ1) alone (FIG. 33A-33B). Bi-cistronic ZF9-MQ1_ ZF3-KRAB reduced total MYC mRNA levels by 99% at 48 hours at both 0.6 μg/ml and 2 μg/ml concentrations (FIG. 33A). Bi-cistronic ZF9-MQ1_ZF3-KRAB reduced the viability of Hep3B cells by about 80% and 27%, respectively, at both 2 μg/ml and 0.6 μg/ml concentrations (FIG. 33B). Furthermore, treatment with the bi-cistronic construct was equally effective to co-formulation of ZF3-KRAB and ZF9-MQ1 constructs.

### Example 33: Bi-Cistronic ZF9-MQ1_ZF3-KRAB Reduces MYC mRNA and HCC Cell Viability in Dose Dependent Manner Across HCC Subtypes

This example evaluates the potency of bi-cistronic ZF9-MQ1_ZF3-KRAB across HCC subtype S1 and S2. HCC S1 subtype cell lines SKHEP-1, SNU-449 and SNU-182 and S2 subtype cell lines Hep 3B and Hep G2 were treated with bi-cistronic ZF9-MQ1_ZF3-KRAB. We evaluated MYC mRNA and cell viability of bi-cistronic construct ZF9-MQ1_ ZF3-KRAB at serially diluted concentrations after 72 hours of treatment.

HCC cells were seeded in 96-well plates in growth media (˜10,000 cells/well). LNP formulations (starting at 2.5 μg/ml) were then added to 3 wells each then diluted ˜1:2 in subsequent wells for 10 doses points in order to transfect mRNA then incubated for 72 hours. Different replicate plates were collected for viability and RNA. Viability was measured using the CellTiter-GLO® assay kit from Promega according to manufacturer's protocol. RNA was isolated from three biological replicates, using the Rneasy® Plus 96-well Kit (Qiagen) following the Manufacturer's protocol. RNA samples were retrotranscribed to cDNA using LunaScript® SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH genes (using TaqMan™ primer/probes) using the ΔΔCt method. The untreated cells were used to normalize expression of MYC.

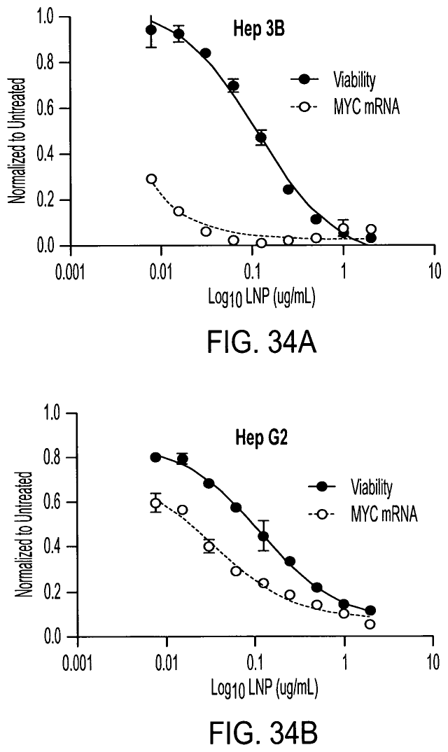

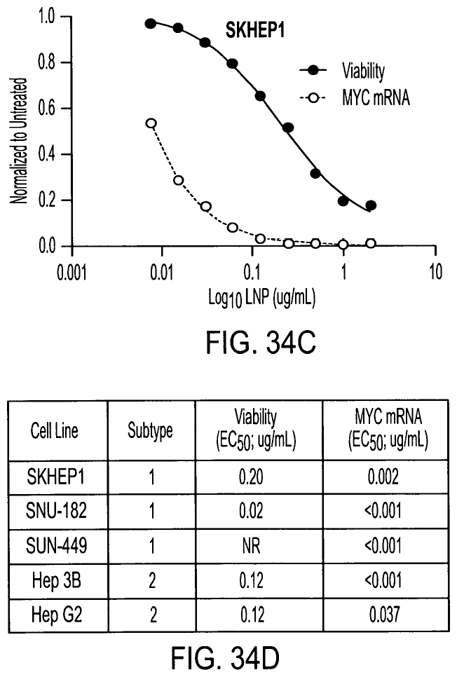

Results indicate that bi-cistronic ZF9-MQ1_ZF3-KRAB treatment showed effects on cell viability across HCC subtypes (FIG. 34A-C). EC50 values for inhibition of MYC mRNA range from <1-20 ng/mL with no trend between S1 and S2 subtypes (FIG. 34D). Similarly, loss of 50% cell viability translated to a higher range of values (120-200 ng/mL) compared to MYC mRNA EC50. There was no significant difference between S1 and S2 subtypes in either MYC mRNA expression or cell viability. EC50 value for three S1 and two S2 subtype HCC tumor cell lines, demonstrated that bi-cistronic ZF9-MQ1_ZF3-KRAB was effective against both HCC subtypes (FIG. 34D).

### Example 34: Apoptosis Induction of HCC Cells by Bi-Cistronic ZE9-MQ1_ZF3-KRAB

This example describes the effect of bi-cistronic ZF9-MQ1_ZF3-KRAB on cellular apoptosis of HCC cells. Viability assays such as CellTiter-GLO® only assess relative number of cells remaining in the well based on levels of ATP not distinguishing between a loss of cell proliferation and cell death.

In this example, fluorescently tagged antibodies to the Annexin V protein (Annexin V FITC) and propidium iodide (PI) was used to quantify apoptotic cells in three HCC cell lines: Hep 3B, Hep G2, and SK-HEP-1 following transfection with bi-cistronic ZF9-MQ1_ZF3-KRAB. A non-coding mRNA was used a negative control in addition to untreated cells. HCC cells were plated in 12 well plates in growth media (50,000 cells per well). LNP formulations (1 μg/ml) were then added to the cells to transfect the mRNAs and incubated for 48 hours. Cells were harvested and stained using the BD Annexin V: FITC apoptosis detection kit (BDB556570) and analyzed by flow cytometry. Cells positive for Annexin V FITC and PI were categorized as apoptotic.

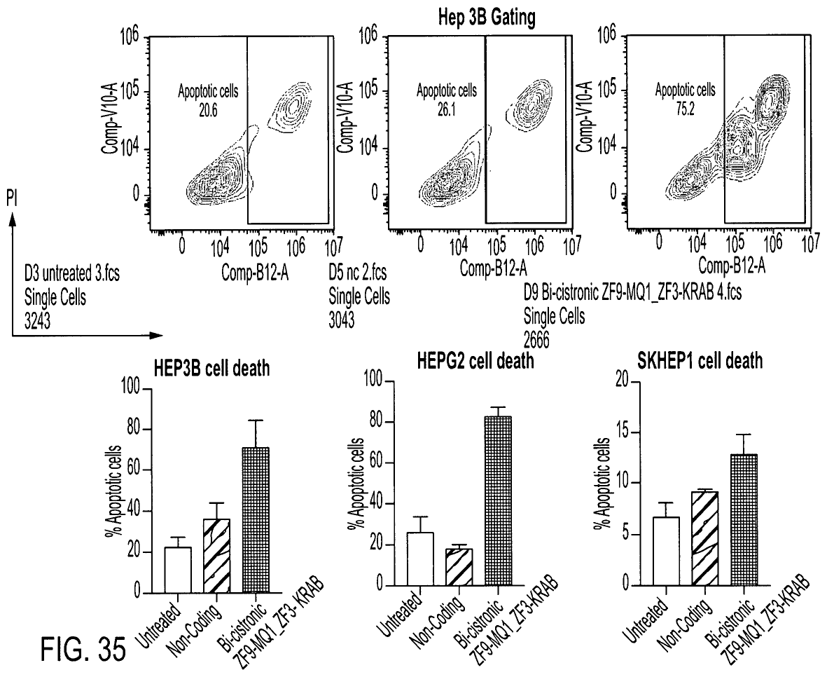

These data showed, at 48 hours of treatment with bi-cistronic ZF9-MQ1 ZF3-KRAB>75% apoptotic cells were detected in the Hep 3B and Hep G2 cell lines and 15% apoptotic cells were detected in the SK-HEP-1 cell line (FIG. 35). Cells were unaffected by non-coding mRNA control compared to untreated cells (5-20% background apoptosis) (FIG. 35). indicating bi-cistronic ZF9-MQ1_ZF3-KRAB was capable of inducing cellular apoptosis of HCC cell lines in culture.

### Example 35: Bi-Cistronic ZF9-MQ1_ZE3-KRAB Reduces MYC mRNA Levels in a Durable Manner

In this example, the durability of bi-cistronic ZF9-MQ1_ZF3-KRAB on MYC mRNA downregulation was evaluated following one treatment of bi-cistronic ZF9-MQ1_ZF3-KRAB SSOP LNPs. Bi-cistronic ZF9-MQ1_ZF3-KRAB represses MYC by directing methylation and repressive histone marks to the MYC IGD. To determine the duration for which those modifications could be effective, SK-HEP-1 cells were utilized as they exhibited minimal effects on cell viability following treatment with bi-cistronic ZF9-MQ1_ZF3-KRAB. The objective of this study was to determine if one treatment of bi-cistronic ZF9-MQ1_ZF3-KRAB could maintain MYC mRNA repression for ˜2 weeks.

SK-HEP-1 cells were plated in a 6 well plate at 200,000 cells per well in 2 mL of growth media. Cells were then treated with 0.6 μg/mL of bi-cistronic ZF9-MQ1_ZF3-KRAB or control non-coding mRNA LNPs. On day 1 post treatment with bi-cistronic ZF9-MQ1_ZF3-KRAB, cells were trypsinized and split into three samples; 1 sample for RNA extraction, 1 sample for genomic DNA (gDNA) extraction, 1 samples saved for future timepoints. This process was repeated for day 3, 6, 9, and 12 post-treatment. For the Day 15, remaining cells were split equally for RNA and gDNA extraction. RNA was isolated from three biological replicates, using the Rneasy® Plus 96-well Kit (Qiagen) following the Manufacturer's protocol. RNA samples were retrotranscribed to cDNA using LunaScript® RT SuperMix Kit (NEB) and analyzed by quantitative PCR (qPCR) (in technical triplicates) using a MYC specific TaqMan™ primer/probe set assay with the TaqMan™ Fast Advanced Master Mix (Thermo Scientific). MYC expression was quantified relative to the expression of GAPDH genes (using TaqMan™ primer/probes) using the ΔΔCt method. The untreated cells were used to normalize expression of MYC.

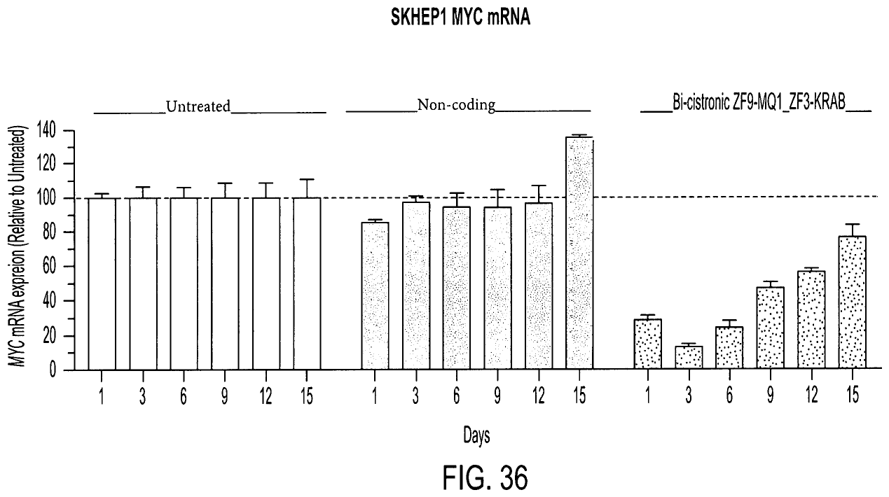

This data demonstrated the durability of bi-cistronic ZF9-MQ1_ZF3-KRAB mediated MYC gene expression modulation. After one treatment of bi-cistronic ZF9-MQ1_ZF3-KRAB in SKHEP1 cells, MYC mRNA levels were reduced at day 1 and remained repressed up to 15 days after treatment (FIG. 36).

### Example 36: Bi-Cistronic ZF9-MQ1_ZF3-KRAB Reduces MYC mRNA and Protein Expression and Cell Viability in HCC Cell Lines

The expression of MYC mRNA and protein levels in HCC cells, cell viability, and expression of bi-cistronic ZF9-MQ1_ZF3-KRAB were assessed at 6-96 hours after treatment with bi-cistronic ZF9-MQ1_ZF3-KRAB to understand the pharmacodynamics of bi-cistronic ZF9-MQ1_ZF3-KRAB in multiple HCC cells.

Hep 3B or SK-HEP-1 cells were plated in sets of two 96-well plates at 10,000 cells per well and a 6-well plate at 400,000 cells per well in culture media for each timepoint. The 96-well plates were treated in triplicate with either bi-cistronic ZF9-MQ1_ZF3-KRAB or non-coding mRNA at 1 μg/mL for cell viability and mRNA analysis. The 6-well plate was treated in duplicate with either bi-cistronic ZF9-MQ1_ZF3-KRAB or non-coding mRNA at 1 μg/mL for protein analysis. Cells were incubated for 6, 24, 48, 72, or 96 hours after treatment, with a subset of cells at each timepoint left as untreated negative controls. At each timepoint, one 96-well plate was lysed with CellTiter-GLO® and luminescence quantified using the GLOMAX®. The second 96-well plate was lysed with RLT Plus Lysis buffer for mRNA extraction using Rneasy® Plus 96 Kit. The lysed sample was bound to an RNA column, washed with buffers, and eluted off. mRNA was then converted to cDNA with RT LunaScript®. cDNA was then analyzed through ΔΔCT qPCR with a MYC (target) and GAPDH (reference) probe. Cells from the 6-well plate were lysed with RIPA buffer for protein isolation at each timepoint. Total protein was quantified using the Pierce BCA protein assay (23225). Equal amounts of protein were loaded for each sample and separated by size using the NuPAGE™ mini gel system (Thermo Fisher). Protein was then transferred to PVDF membrane using the iBlot™ 2 gel transfer device (Thermo Fisher). Membranes were probed overnight with anti-MYC antibody (Abcam ab32072). Anti-β-actin antibody (Cell Signaling 8H10D10) was used as a loading control. Signal was then visualized and quantified using the LI-COR imaging system using fluorescent secondary antibodies to the MYC and β-actin primary antibodies.

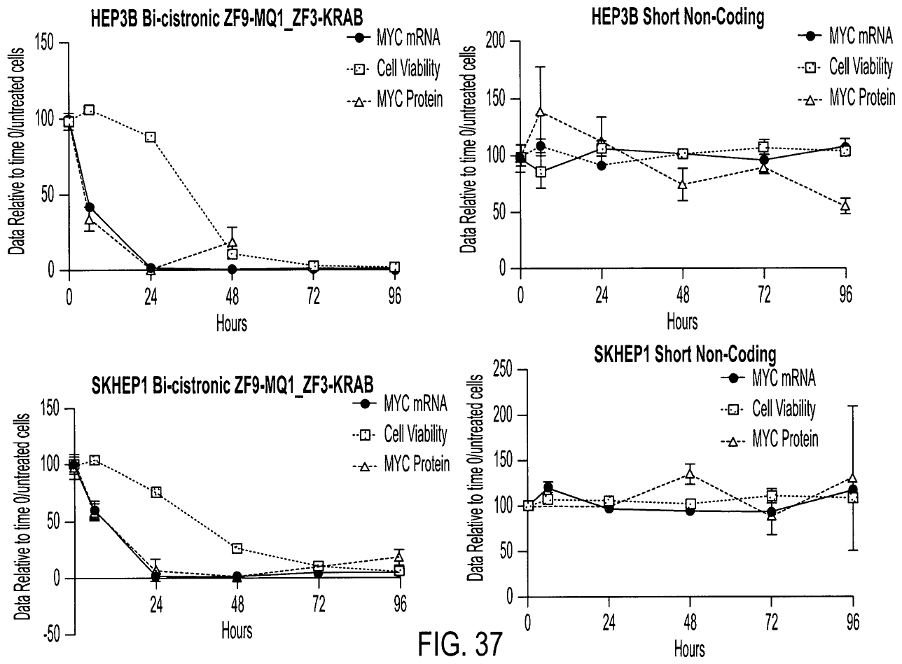

In both cell lines, bi-cistronic ZF9-MQ1_ZF3-KRAB decreased MYC mRNA and protein expression at 6 hours which remained down 96 hours later when compared to short non-coding mRNA or untreated cells (FIG. 37). In both cell lines, a decrease in cell viability was observed 48 hours after treatment with bi-cistronic ZF9-MQ1_ZF3-KRAB (FIG. 37). The short non-coding negative control had no effect on cell viability.

### Example 37: Bi-Cistronic ZF9-MQ1_ZF3-KRAB mRNA Expresses Both ZF9-MQ1 and ZF3-KRAB as Visualized by HA Tagged Proteins

In this example, expression of the proteins encoded by bi-cistronic ZF9-MQ1_ZF3-KRAB mRNA/LNPs was confirmed through western blot analysis. Inside a cell, a bi-cistronic ZF9-MQ1_ZF3-KRAB produces two ZF proteins (ZF3-KRAB and ZF9-MQ1) which, in this experiment, are tagged with HA, allowing quantification of protein expression following transfection of HCC cells.

Hep 3B or SK-HEP-1 cells were plated in a 6-well plate at 400,000 cells per well in culture media for each timepoint. The 6-well plate was treated in duplicate with bi-cistronic ZF9-MQ1_ZF3-KRAB at 1 μg/mL for protein analysis. Cells were incubated for either 6 or 24 hours. Cells were then lysed in RIPA buffer and total protein levels quantified using the Pierce BCA protein assay (23225). Equal amounts of protein were loaded for each sample and separated by size using the NuPAGE™ mini gel system (Thermo Fisher). Protein was then transferred to PVDF membrane using the iBlot™ 2 gel transfer device (Thermo Fisher). Membranes were probed overnight with Abcam HA antibody. Anti-β-actin antibody (Cell Signaling 8H10D10) was used as a loading control. Signal was visualized and quantified using the LI-COR imaging system using fluorescent secondary antibodies to the MYC and β-actin antibodies.

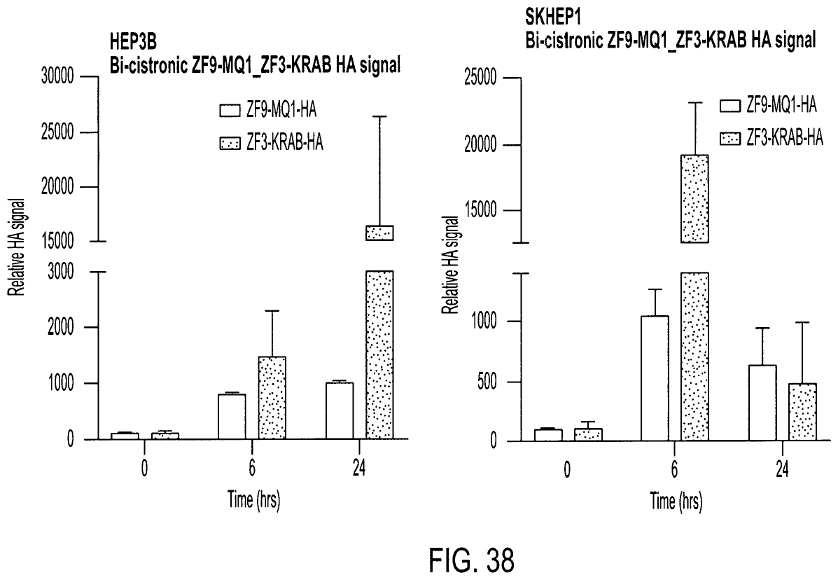

At both 6 and 24 hours following transfection, both ZF3-KRAB and ZF9-MQ1 proteins encoded by bi-cistronic ZF9-MQ1_ZF3-KRAB mRNA were visualized by HA tag on a western blot (FIG. 38). Accumulation of both ZF3-KRAB and ZF9-MQ1 constructs was observed at both time points.

### Example 38: Bi-Cistronic ZF9-MQ1_ZF3-KRAB Increases Sorafenib Response in HCC Cells

In this example, the effect of bi-cistronic ZF9-MQ1_ZF3-KRAB on the small molecule sorafenib (multi-kinase inhibitor) efficacy in HCC cell lines was evaluated. Sorafenib is used as a standard of care in HCC and high MYC levels can predict sorafenib resistance. It was hypothesized that bi-cistronic ZF9-MQ1_ZF3-KRAB may re-sensitize HCC to sorafenib through its downregulation of MYC. Dose response assays were utilized to evaluate if bi-cistronic ZF9-MQ1_ZF3-KRAB treatment could decrease the IC₅₀ of sorafenib.

To evaluate potential synergy between bi-cistronic ZF9-MQ1_ZF3-KRAB and sorafenib, Hep 3B or SK-HEP-1 cells were plated in 96 well plates at 10,000 cells per well. Cells were then treated with sorafenib at a dose range between 0.1 and 25 μM, with serial dilution of 1:2. Lipid mix carrying bi-cistronic ZF9-MQ1_ZF3-KRAB was then added to a subset of the wells at a dose of 0.1 or 0.6 μg/mL. A set of cells was treated with sorafenib alone to use as control. Following 72-hour treatment, cells were lysed with the CellTiter-GLO® reagent and luminescence was quantified using the Glo Max. Relative cell viability was calculated by averaging the untreated values and dividing each experimental luciferase value by that average.

TABLE 19
Effect of bi-cistronic ZF9-MQ1_ZF3-KRAB on sorafenib IC₅₀

| Hep3B | | SKHEP-1 | |
|---|---|---|---|
| bi-cistronic ZF9- | | bi-cistronic ZF9- | |
| MQ1_ZF3-KRAB | Sorafenib | MQ1_ZF3-KRAB | Sorafenib |
| concentration | IC₅₀ | concentration | IC₅₀ |
| 0 μg/ml | 4.4 μM | 0 μg/ml | 12.3 μM |
| 0.1 μg/ml | 4.25 μM | 0.1 μg/ml | 12.3 μM |
| 0.6 μg/ml | 2.9 μM | 0.6 μg/ml | 10.7 μM |

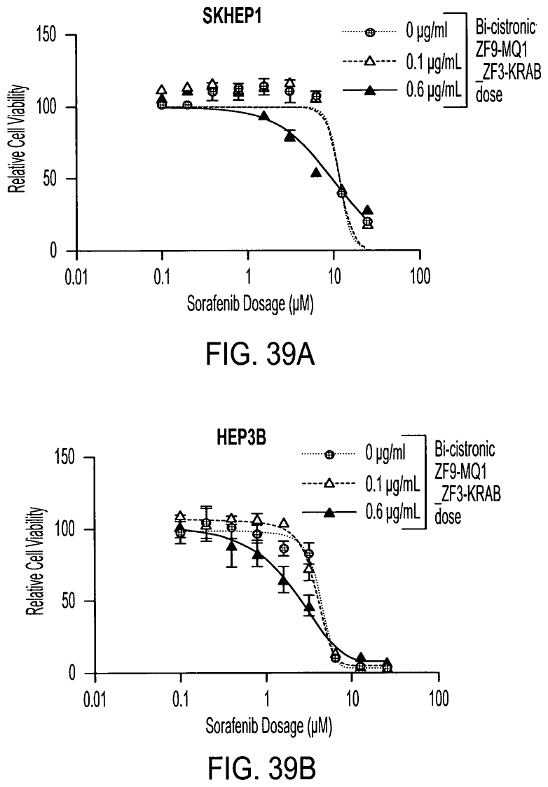

The data showed that the IC₅₀ of sorafenib in SKHEP1 reduced from 12.3 to 10.7 μM (FIG. 39A and Table 19) and in Hep 3B reduced from 4.4 to 2.9 μM (FIG. 39B and Table 19) when sorafenib was administered in combination with 0.6 μg/ml bi-cistronic ZF9-MQ1_ZF3-KRAB. the IC₅₀ of sorafenib did not change significantly in Hep 3B or SK-HEP-1 cells when sorafenib was administered in combination with 0.1 μg/ml bi-cistronic ZF9-MQ1_ZF3-KRAB (FIG. 39A-39B). The combination of sorafenib and bi-cistronic ZF9-MQ1_ZF3-KRAB was more effective than sorafenib alone (FIG. 39A-39B). This data suggested synergistic activity between bi-cistronic ZF9-MQ1_ZF3-KRAB and sorafenib.

### Example 39: Bi-Cistronic ZF9-MQ1_ZF3-KRAB Increases the JQ1 Response in HCC Cells

This example evaluates the efficacy of bi-cistronic ZF9-MQ1_ZF3-KRAB in combination with JQ1 (BET inhibitor) in multiple HCC cell lines. BET proteins have been showed to be important for MYC transcription. Later generation BET inhibitors are currently being evaluated in the clinic for hematologic indications; however, their toxicity profile have limited their use. Combination treatments that can increase the potency of BET inhibitors at reduced dose levels could improve BET inhibitor tolerability.

To evaluate potential synergy between bi-cistronic ZF9-MQ1_ZF3-KRAB and JQ1, Hep 3B or SK-HEP-1 cells were plated in 96 well plates at 10,000 cells per well. Cells were then treated with JQ1 with a dose range between 0.1 and 25 μM, with serial dilution of 1:2. Lipid mix carrying bi-cistronic ZF9-MQ1_ZF3-KRAB was then added to a set of the wells at a dose of 0.1 or 0.6 μg/mL. A set of cells was treated with JQ1 alone to use as control. Following 72-hour treatment, cells were lysed with the CellTiter-GLO® reagent and luminescence was quantified using the Glo Max. Relative cell viability was calculated by averaging the untreated values and dividing each experimental luciferase value by that average.

TABLE 20
Effect of bi-cistronic ZF9-MQ1 ZF3-KRAB on JQ1 IC50

| Hep3B | | SKHEP-1 | |
|---|---|---|---|
| bi-cistronic ZF9- | | bi-cistronic ZF9- | |
| MQ1_ZF3-KRAB | JQ1 | MQ1_ZF3-KRAB | JQ1 |
| concentration | IC₅₀ | concentration | IC₅₀ |
| 0 μg/ml | 6.6 μM | 0 μg/ml | >25 μM |
| 0.1 μg/ml | 0.6 μM | 0.1 μg/ml | 1.9 μM |
| 0.6 μg/ml | 0.2 μM | 0.6 μg/ml | 1.1 μM |

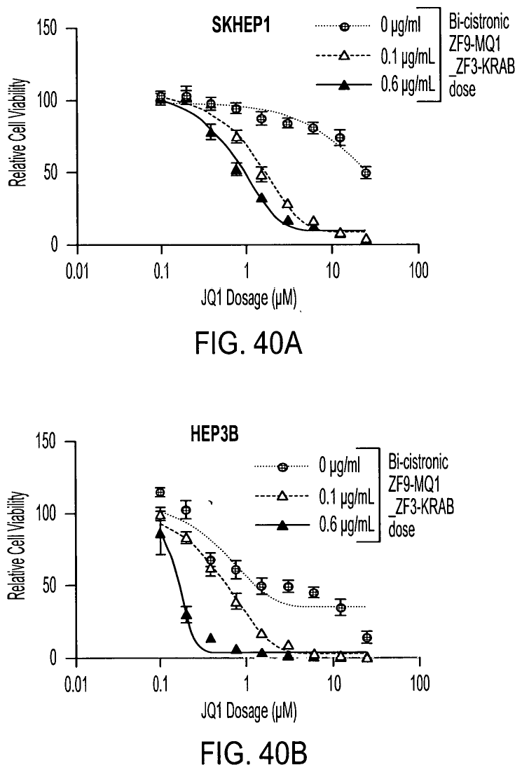

The combination of 0.6 μg/mL of bi-cistronic ZF9-MQ1_ZF3-KRAB plus JQ1 reduced the IC₅₀ of JQ1 in SK-HEP1 from >25 to 1.1 μM (FIG. 40A and Table 20) and in Hep 3B from 6.6 to 0.2 p′M (FIG. 40B and Table 20). The combination of 0.1 μg/mL of bi-cistronic ZF9-MQ1_ZF3-KRAB plus JQ1 reduced the IC₅₀ of JQ1 in SK-HEP1 from >25 to 1.9 μM (FIG. 40A and Table 20) and in Hep 3B from 6.6 to 0.6 μM (FIG. 40B and Table 20). This data suggested synergistic activity between bi-cistronic construct ZF9-MQ1_ZF3-KRAB and JQ1.

### Example 40: Screening Mouse Surrogate Constructs Designed to Target the MYC Genomic Locus

This example, a mouse model of hepatocellular carcinoma, Hepa1-6, was used to evaluate constructs that target to the MYC IGD in mouse. These constructs (ZF15-MQ1, ZF16-MQ1 and ZF17-MQ1) were developed as surrogates to bi-cistronic ZF9-MQ1_ZF3-KRAB that target the mouse genome. We evaluated the ability of these constructs to downregulate MYC mRNA expression and reduce mouse HCC cell viability.

A panel of mouse surrogate constructs (ZF15-MQ1, ZF16-MQ1 and ZF17-MQ1) were generated and screened in Hepa1-6 cells by seeding 10,000 cells per well in duplicate plates for mRNA extraction or cell viability analysis. The 96-well plates were treated in triplicate with the candidate ZFs at 0.6 or 1.2 μg/mL. Cells were incubated for 72 hours. Following incubation period, one 96-well plate was lysed with CellTiter-GLO® and luminescence was quantified using the GloMax® Discovery plate reader. The second 96-well plate was lysed with RLT Plus Lysis buffer for mRNA extraction using Rneasy® Plus 96 Kit. The lysed sample was bound to an RNA column, washed with buffers, and eluted off. mRNA was then converted to cDNA with RT LunaScript®. cDNA was then analyzed through ΔΔCT qPCR with a MYC (target) and GAPDH (reference) probe.

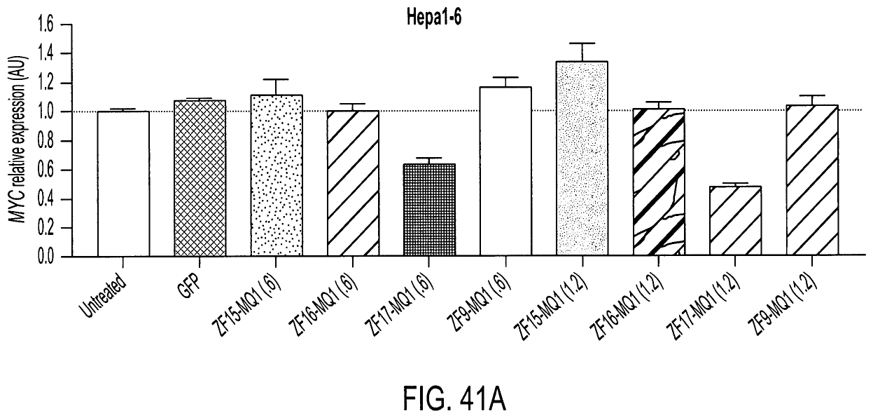

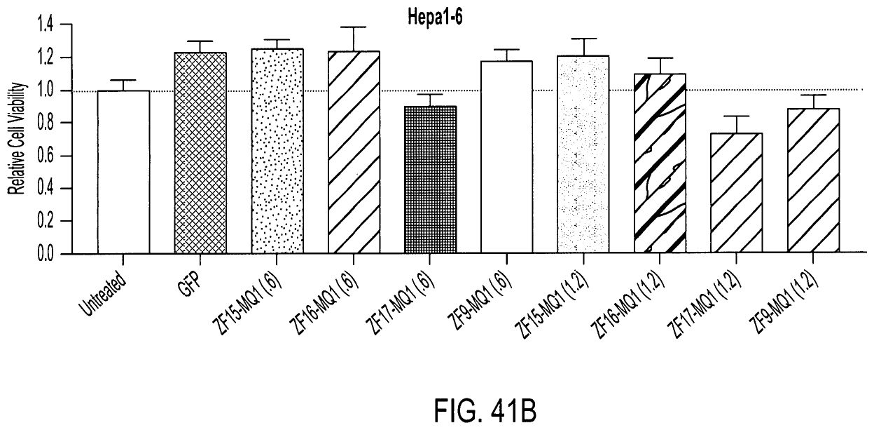

This screen indicated that ZF17-MQ1 showed downregulation of mouse MYC mRNA (FIG. 41A) corresponding to a decrease in viability (FIG. 41B).

### Example 41: Effect of Mouse Surrogate Construct ZF17-MQ1 on HCC Cell Viability and MYC mRNA and Protein Expression

In this example, a mouse model of hepatocellular carcinoma, Hepa1-6, was used to evaluate effect of ZF17-MQ1 construct downregulating MYC mRNA and protein expression and mouse HCC cell viability.

MYC mRNA and cell viability were analyzed 96 hours of treatment with ZF17-MQ1 or GFP mRNA. Hepa1-6 cells were seeded at 10,000 cells per well in duplicate plates for mRNA extraction or cell viability analysis. The 96-well plates were treated in triplicate with ZF17-MQ1 at 0.6 or 1.2 μg/mL. After incubation, one 96-well plate was lysed with CellTiter-GLO® and luminescence was quantified using the GloMax® Discovery plate reader. The second 96-well plate was lysed with RLT Plus Lysis buffer for mRNA extraction using RNeasy® Plus 96 Kit. The lysed sample was bound to an RNA column, washed with buffers, and eluted off. mRNA was then converted to cDNA with RT Lunascript. cDNA was then analyzed through OCTA qPCR with a MYC (target) and GAPDH (reference) probe.

MYC protein levels were analyzed after 24 and 48 hours of treatment by transfecting 100,000 cells in a 12 well plate with ZF17-MQ1 or control GFP mRNA. Cells were lysed in RIPA buffer and total protein levels were quantified using the Pierce BCA protein assay (23225). Equal amounts of protein were loaded for each sample and separated by size using the NuPAGE™ mini gel system (Thermo Fisher). Protein was then transferred to PVDF membrane using the iBlot™ gel transfer device (Thermo Fisher). Membranes were probed overnight with anti-MYC antibody (Abcam ab32072). Anti-β-actin antibody (Cell Signaling 8H10D10) was used as a loading control. Signal was then visualized and quantified using the LI-COR imaging system using fluorescent secondary antibodies to the MYC and β-actin antibodies.

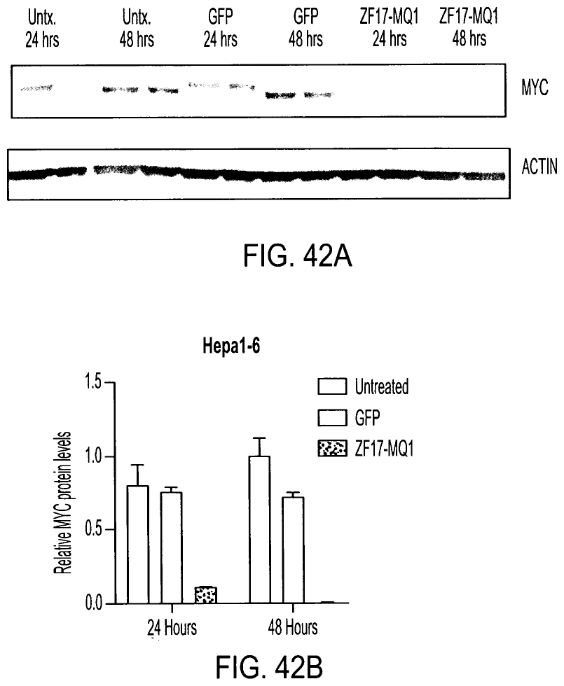

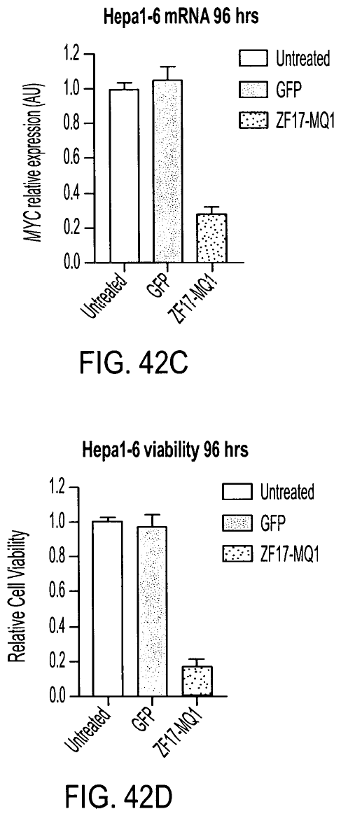

The data showed that ZF17-MQ1 was able to function as a mouse surrogate for bi-cistronic ZF9-MQ1_ZF3-KRAB by targeting the MYC IGD. ZF17-MQ1 treatment in mouse HCC cells Hepa1-6 showed significant downregulation of MYC protein at 24 and 48 hours (FIG. 42A). ZF17-MQ1 treatment in mouse HCC cells Hepa1-6 showed significant downregulation of MYC mRNA at 96 hours (FIG. 42C) and decrease in cell viability at 96 hours (FIG. 42D).

### Example 42: Hepa1-6 Subcutaneous Syngeneic Model Showing Efficacy of ZF17-MQ1

This example demonstrates ZF17-MQ1 efficacy in an immune-competent animal. Specifically, Hepa1-6 is implanted into syngeneic recipient C57BL/6 normal mice.

Disease was induced in female C57BL/6 mice by the implantation of Hepa1-6 tumor cells into the left flank. Treatment was initiated when mean tumor volume reached ˜150 mm³. Mice were divided into treatment groups (9 mice each for PBS or ZF17-MQ1, 6 mice for sorafenib) so that mean tumor volume in each group was approximately equal. Mice were injected intravenously with PBS or ZF17-MQ1 at 3 mg/kg. The positive control standard of care drug sorafenib at 50 mg/kg was given via oral gavage every day. ZF17-MQ1 was dosed every 5 days for 4 doses then given a 2-week drug holiday with treatment re-initiated twice more. All animals were weighed daily and assessed visually. Tumor size were measured via calipers 3 times per week.

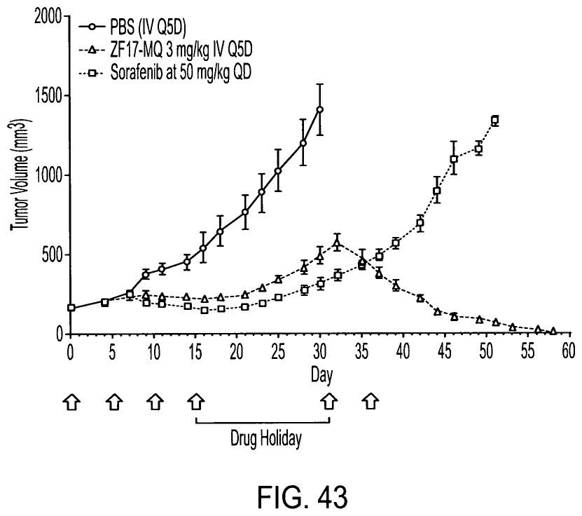

The results showed that ZF17-MQ1 significantly reduced animal tumor burden after 4 doses (FIG. 43). Following a drug holiday, re-treatment of the mice resulted in full tumor depletion after ˜4 weeks (FIG. 43). These data showed that ZF17-MQ1 could effectively reduce tumor burden in HCC xenografts done in immune-competent animals.

### Example 43: Epigenetic Modulation of the MYC Oncogene as a Potential Novel Therapy for HCC

This example describes characterizing bi-cistronic ZF9-MQ1-ZF3-KRAB in HCC cell lines (Hep 3B, Hep G2, SK-HEP-1, SNU-182 and SNU-449) by measuring MYC mRNA and cell viability.

The bi-cistronic ZF9-MQ1_ZF3KRAB construct was characterized in HCC cell lines (Hep 3B, Hep G2, SK-HEP-1, SNU-182 and SNU-449) by measuring MYC mRNA and cell viability. Bi-cistronic ZF9-MQ1_ZF3KRAB was tested for durable epigenetic (e.g., DNA/chromatin methylation) and transcriptome (e.g., RNA-seq) changes. Changes in MYC protein levels and pathway signaling were measured using various proteomic methods. Finally, the activity of bi-cistronic ZF9-MQ1_ZF3KRAB were analyzed in-vivo in subcutaneous (subQ) and orthotopic HCC models by assessing tumor volume, tumor-associated bioluminescence (BLI) and immunohistochemistry (IHC).

We identified constructs, including bi-cistronic ZF9-MQ1_ZF3-KRAB, that target multiple loci on the MYC IGD and that were effective at decreasing MYC mRNA, protein and cell viability in HCC cells while sparing normal cells. In HCC cells, bi-cistronic ZF9-MQ1.ZF3KRAB median EC50 of inhibition is <0.001 ng/mL for MYC mRNA and 120 ng/mL for cell viability. Importantly, the effects of bi-cistronic ZF9-MQ1_ZF3KRAB persisted for over 2 weeks providing durable MYC mRNA repression. Intravenous delivery of bi-cistronic ZF9-MQ1_ZF3KRAB in LNPs at 3 and 6 mg/kg Q5D in a Hep 3B subQ model in athymic nude mice demonstrated a statistically significant tumor growth inhibition (TGI) of 54% and 63%, respectively, by Day 23 compared to negative control. Mice treated with bi-cistronic ZF9-MQ1_ZF3KRAB did not have a significant decrease in bodyweight (BW) compared to negative control or sorafenib-treated mice. IHC of bi-cistronic ZF9-MQ1_ZF3KRAB and control treated tumors showed significant down-regulation of MYC and Ki67, a marker of proliferation, and upregulation of Caspase 3, a marker of apoptosis. In a Hep 3B orthotopic model 3 mg/kg bi-cistronic ZF9-MQ1_ZF3KRAB Q5D showed a comparable reduction of BLI to sorafenib at 50 mg/kg QD without the reduction in BW.

### Example 44: Effect of Mouse Surrogate Constructs on MYC Expression and Cell Viability in LL2 Cells

The Example is directed to evaluating the effect of mouse surrogate constructs (ZF15-MQ1, ZF16-MQ1, and ZF17-MQ1) on MYC expression and cell viability in LL2 cells.

LL2 cells were seeded at 5,000 cells per well in duplicate 96 well plates for mRNA extraction or cell viability analysis. The 96-well plates were treated in triplicate with ZF15-MQ1, ZF16-MQ1, or ZF17-MQ1 loaded LNPs at 0.625 μg/mL (for mRNA and viability readouts) and at 1.25 μg/ml (mRNA and viability readouts).and incubated. 6 well plates were seeded with 200,000 cells per well and transfected with the above constructs at 1.25 μg/ml (for western blot readout). GFP treated cells were used as negative control. After incubation, one 96-well plate was lysed with CellTiter-GLO® and luminescence was quantified using the GloMax® Discovery plate reader. The second 96-well plate was lysed with RLT Plus Lysis buffer for mRNA extraction using Rneasy® Plus 96 Kit. The lysed sample was bound to an RNA column, washed with buffers, and eluted off mRNA was then converted to cDNA using poly-A primer with RT Lunascript. cDNA was then used for reverse transcription polymerase chain reaction (RT-PCR) analysis using TaqMan™ probes (Thermo Fisher) specific for mouse MYC mRNA transcripts. GAPDH mRNA transcript levels were used for normalization across groups. For western blot, β-actin antibody was stained with a secondary antibody tagged with fluorophore emitting at 594 nm wavelength and MYC antibody (Abcam) was detected with secondary antibody tagged with fluorophore emitting at a wavelength of 488 nm. Odyssey® CLx Imaging System (LI-COR) using near-infrared (NIR) fluorescence was used to capture the protein images.

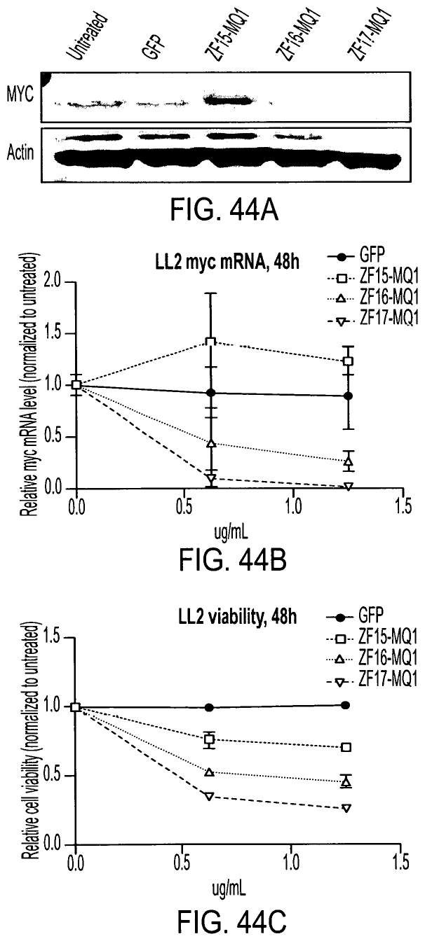

The data showed that ZF17-MQ1 treated cells showed reduced MYC protein levels in LL2 cells in comparison to untreated or GFP-treated conditions cells (FIG. 44A). Compared to levels observed in untreated cells, ZF17-MQ1 and ZF16-MQ1 reduced MYC mRNA levels by >99.9% or 74%, respectively in LL2 cells (FIG. 44B). Further, all three constructs were able to reduce cell viability in LL2 cell to a greater extent than untreated and GFP-treated cells (FIG. 44C). Compared to untreated cells, constructs ZF17-MQ1, ZF16-MQ1, and ZF15-MQ1 reduced viability up to 74%, 65%, and 30%, respectively in LL2 cells.

### Example 45: MYC Transcript is Downregulated in ZF17-MQ1 Treated LL2 and CT26 Cells

This example is directed to evaluation of the efficacy of ZF17-MQ1 construct in downregulating MYC mRNA expression and reducing cell viability in CT26 and LL2 cells.

CT26 and LL2 cells were seeded at 2,500 cells per well in duplicate plates for mRNA extraction or cell viability analysis. The 96-well plates were treated in triplicate with ZF17-MQ1 loaded LNPs at 1.25 μg/mL and 2.5 μg/ml and incubated. Untreated cells and GFP treated cells were used as controls. After incubation, one 96-well plate was lysed with CellTiter-GLO® and luminescence was quantified using the GloMax® Discovery plate reader. The second 96-well plate was lysed with RLT Plus Lysis buffer for mRNA extraction using Rneasy® Plus 96 Kit. The lysed sample was bound to an RNA column, washed with buffers, and eluted off. mRNA was then converted to cDNA using poly-A primer with RT Lunascript. cDNA was then used for reverse transcription polymerase chain reaction (RT-PCR) analysis using TaqMan™ probes (Thermo Fisher) specific for mouse MYC mRNA transcripts. GAPDH mRNA transcript levels were used for normalization across groups.

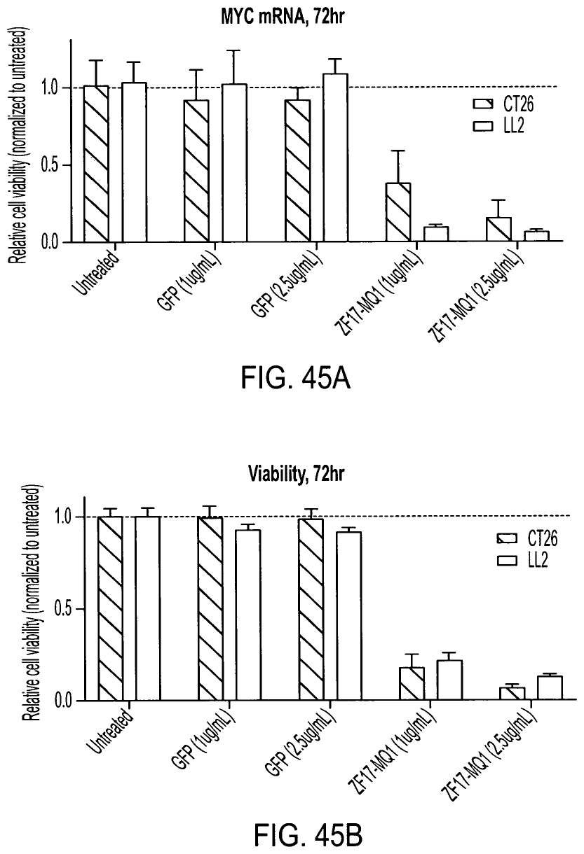

The data showed ZF17-MQ1 downregulated MYC mRNA and reduced cell viability in LL2 and CT26 cells to a greater extent than untreated and GFP-treated cells (negative control). Compared to levels observed in untreated cells, at 2.5 μg/mL ZF17-MQ1 reduced MYC mRNA levels by 93% and 85% in LL2 and CT26 cells, respectively (FIG. 45A). Furthermore, compared to untreated cells, under these conditions, ZF17-MQ1 reduced cell viability by 87% and 93% in LL2 and CT26 cells, respectively (FIG. 45B).

### Example 46: MYC Transcript is Downregulated in ZF17-MQ1 Treated LL2 and CMT167 Cells

This example is directed to evaluation of the efficacy of ZF17-MQ1 construct in downregulating MYC mRNA expression and reducing cell viability in CMT167 and LL2 cells.

CMT167 and LL2 cells were seeded at 5,000 cells per well in duplicate plates for mRNA extraction or cell viability analysis. The 96-well plates were treated in triplicate with GFP, non-coding RNA, or ZF17-MQ1 loaded LNPs at 1.0 μg/mL and incubated. Untreated cells and GFP treated cells were used as controls. After incubation, one 96-well plate was lysed with CellTiter-GLO® and luminescence was quantified using the GloMax® Discovery plate reader. The second 96-well plate was lysed with RLT Plus Lysis buffer for mRNA extraction using Rneasy® Plus 96 Kit. The lysed sample was bound to an RNA column, washed with buffers, and eluted off. mRNA was then converted to cDNA using poly-A primer with RT Lunascript. cDNA was then used for reverse transcription polymerase chain reaction (RT-PCR) analysis using TaqMan™ probes (Thermo Fisher) specific for mouse MYC mRNA transcripts. GAPDH mRNA transcript levels were used for normalization across groups.

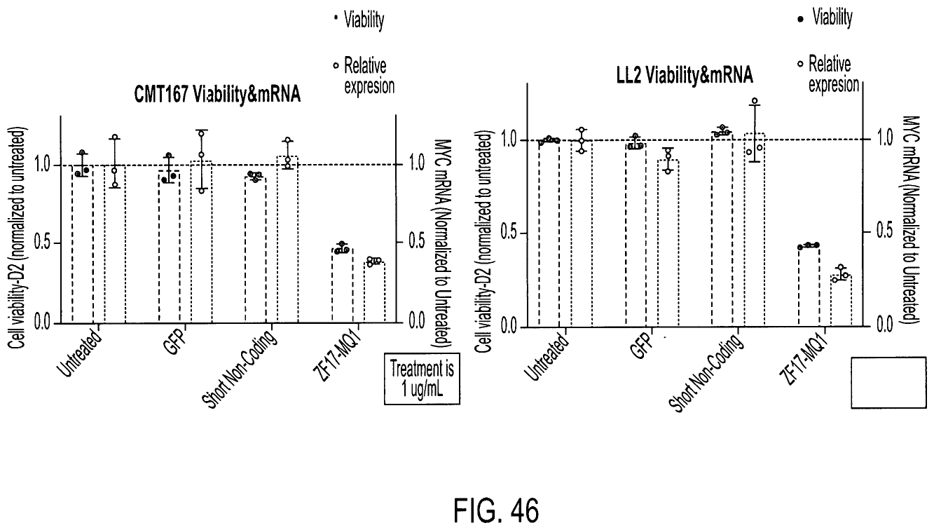

The data showed that ZF17-MQ1 downregulated MYC mRNA and reduces cell viability in CMT167 and LL2 cells to a greater extent than untreated and GFP-treated cells (negative, control). Compared to levels observed in untreated cells, ZF17-MQ1 reduced MYC mRNA levels by 62% and 73% in CMT167 and LL2 cells, respectively (FIG. 46). Furthermore, compared to untreated cells, under these conditions, ZF17-MQ1 reduced cell viability by 54% and 57% in CMT167 and LL2 cells, respectively (FIG. 46).

### Example 47: ZF9-MQ1 Shows Little Effect on Primary Cell Viability

This example evaluates the effect of ZF9-MQ1 construct on primary cell viability.

Primary small airway epithelial cells, primary lobar epithelial cells, and primary lung fibroblast cells were seeded at 7,500 (primary small airway epithelial) or 5000 (primary lobar epithelial and primary lung fibroblast) cells per well in duplicate plates for cell viability analysis. The 96-well plates were treated in triplicate with GFP or ZF9-MQ1 loaded LNPs at 1.0 ug/mL and incubated. Untreated cells and GFP treated cells were used as controls. After incubation, one 96-well plate was lysed with CellTiter-GLO® and luminescence was quantified using the GloMax® Discovery plate reader. The second 96-well plate was lysed with RLT Plus Lysis buffer for mRNA extraction using Rneasy® Plus 96 Kit. The lysed sample was bound to an RNA column, washed with buffers, and eluted off. mRNA was then converted to cDNA using poly-A primer with RT Lunascript. cDNA was then used for reverse transcription polymerase chain reaction (RT-PCR) analysis using TaqMan′ probes (Thermo Fisher) specific for human MYC mRNA transcripts. GAPDH mRNA transcript levels were used for normalization across groups.

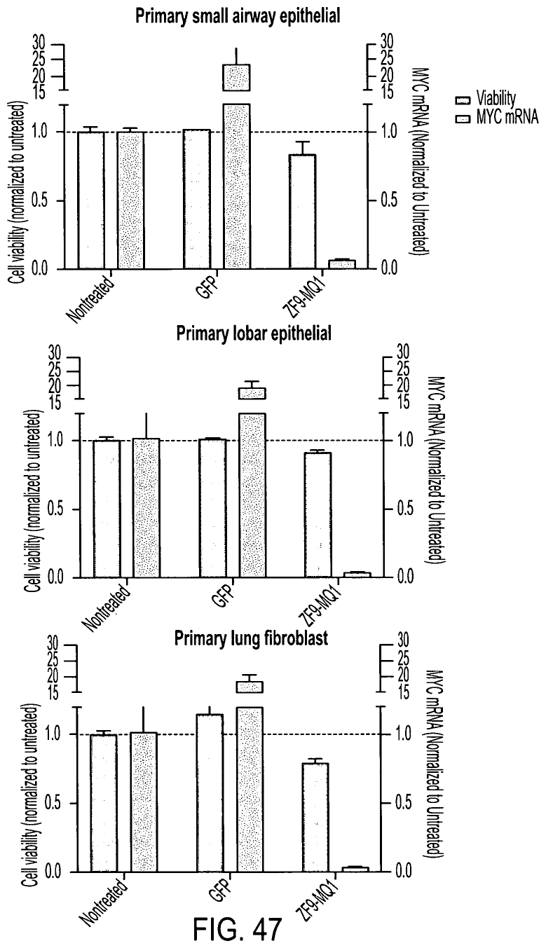

The data showed that ZF9-MQ1 downregulated MYC mRNA levels by 94%, 96%, 96% levels compared to untreated cells in primary small airway epithelial cells, primary lobar epithelial cells, and primary lung fibroblasts respectively (FIG. 47). However, viability was only reduced by 16%, 9%, and 22% compared to control cells suggesting ZF9-MQ1 had only a modest effect on cell viability of normal lung epithelial or fibroblast cells in contrast to previous findings in H2009 cancer cells.

### Example 48: Co-Treatment of ZF9-MQ1 with JQ1 Showed a Greater than Additive Effect on A549 Viability

This example evaluates the effect of ZF9-MQ1 construct on A549 cell viability when used in combination with varying concentrations of JQ1 inhibitor.

A549 cells were seeded at 4,000 cells per well in duplicate plates for cell viability analysis. The 96-well plates were treated in triplicate with GFP or ZF9-MQ1 loaded LNPs at 0.5 ug/mL or 1.0 μg/mL in combination with increasing concentrations of the BET inhibitor JQ1 (concentrations up to 6.25 uM) and incubated. Untreated cells and GFP treated cells were used as controls. After 72-hour incubation, one 96-well plate was lysed with CellTiter-GLO® and luminescence was quantified using the GloMax® Discovery plate reader.

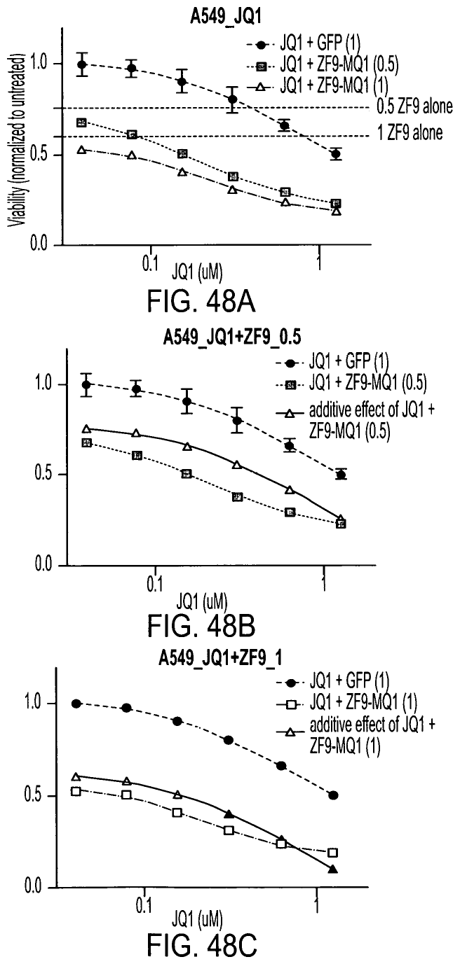

The data showed ZF9-MQ1 and JQ1 each separately inhibited cell viability of A549 cells (FIG. 48A). When combined, ZF9-MQ1 (0.5 or 1 μg/ml) and JQ1 (concentrations up to 6.25 uM) showed a greater than additive effect on the inhibition of A549 viability than what is predicted by their individual activities suggesting that ZF9-MQ1 and JQ1 combination was synergistic to inhibit viability in A549 cells (FIG. 48B-48C).

### Example 49: Co-Treatment of ZF9-MQ1 with BET762 Showed a Greater than Additive Effect on A549 Viability

This example evaluates the effect of ZF9-MQ1 construct on A549 cell viability when used in combination with varying concentrations of BET762 inhibitor.

A549 cells were seeded at 4,000 cells per well in duplicate plates for cell viability analysis. The 96-well plates were treated in triplicate with GFP or ZF9-MQ1 loaded LNPs at 0.5 μg/mL or 1.0 μg/mL in combination with increasing concentrations (concentrations up to 1.25 uM) of the BET inhibitor BET762 and incubated. Untreated cells and GFP treated cells were used as controls. After 72-hour incubation, one 96-well plate was lysed with CellTiter-GLO® and luminescence was quantified using the GloMax® Discovery plate reader.

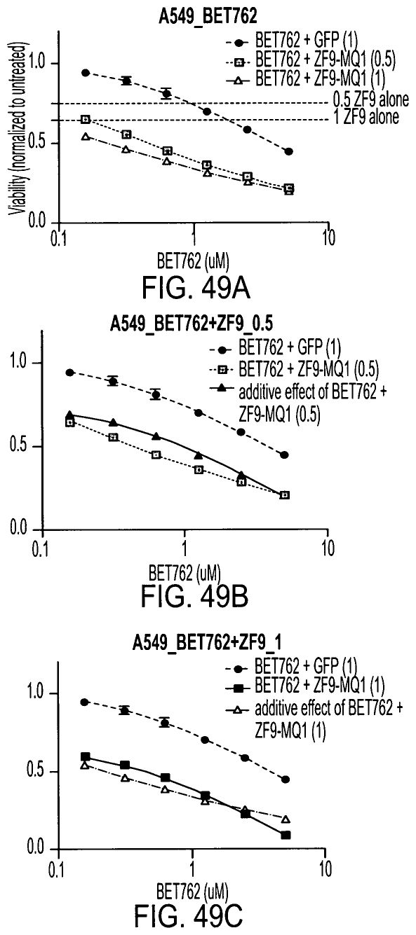

The data showed ZF9-MQ1 and BET762 each separately inhibited cell viability of A549 cells (FIG. 49A). When combined, ZF9-MQ1 (0.5 μg/ml) and BET762 (concentrations up to 1.25 uM for 0.5 μg/ml ZF9-MQ1 treated cells and up to 0.625 uM for 1.0 μg/ml ZF9-MQ1 treated cells) showed a greater than additive effect on the inhibition of A549 viability than what is predicted by their individual activities (FIG. 49B). When combined, ZF9-MQ1 (1.0 μg/ml) and BET762 (concentrations up to 0.625 uM) showed a greater than additive effect on the inhibition of A549 viability (FIG. 49C) than what is predicted by their individual activities. The data suggested that ZF9-MQ1 and BET762 combination was synergistic to inhibit viability in A549 cells (FIG. 49B-C).

### Example 50: Co-Treatment of ZF9-MQ1 with Birabresib Showed a Greater than Additive Effect on A549 Viability

This example evaluates the effect of ZF9-MQ1 construct on A549 cell viability when used in combination with varying concentrations of BET inhibitor, Birabresib.

A549 cells were seeded at 4,000 cells per well in duplicate plates for cell viability analysis. The 96-well plates were treated in triplicate with GFP or ZF9-MQ1 loaded LNPs at 0.5 μg/mL or 1.0 μg/mL in combination with increasing concentrations (concentrations up to 0.625 uM for 0.5 μg/ml ZF9-MQ1 treated cells and up to 0.313 uM for 1.0 μg/ml ZF9-MQ1 treated cells) of the BET inhibitor Birabresib and incubated. Untreated cells and GFP treated cells were used as controls. After 72-hour incubation, one 96-well plate was lysed with CellTiter-GLO® and luminescence was quantified using the GloMax® Discovery plate reader.

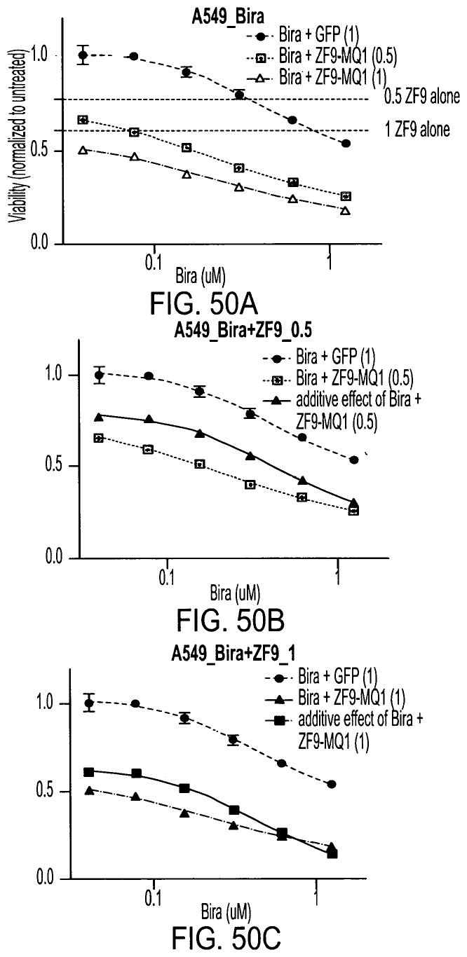

The data showed ZF9-MQ1 and Birabresib each separately inhibited cell viability of A549 cells (FIG. 50A). When combined, ZF9-MQ1 (0.5 μg/ml) and Birabresib (concentrations up to 0.625 uM) showed a greater than additive effect on the inhibition of A549 viability than what is predicted by their individual activities (FIG. 50B). When combined, ZF9-MQ1 (1.0 μg/ml) and Birabresib (concentrations up to 0.313 uM) showed a greater than additive effect on the inhibition of A549 viability (FIG. 50C) than what is predicted by their individual activities. The data suggested that ZF9-MQ1 and Birabresib combination was synergistic to inhibit viability in A549 cells (FIG. 50B-50C).

### Example 51: Co-Treatment of ZF9-MQ1 with Trametinib Showed a Greater than Additive Effect on A549 Viability

This example evaluates the effect of ZF9-MQ1 construct on A549 cell viability when used in combination with varying concentrations of MEK inhibitor, Trametinib.

A549 cells were seeded at 4,000 cells per well in duplicate plates for cell viability analysis. The 96-well plates were treated in triplicate with GFP or ZF9-MQ1 loaded LNPs at 0.5 μg/mL or 1.0 μg/mL in combination with increasing concentrations (concentrations up to 0.05 uM) of the MEK inhibitor Trametinib and incubated. Untreated cells and GFP treated cells were used as controls. After 72-hour incubation, one 96-well plate was lysed with CellTiter-GLO® and luminescence was quantified using the GloMax® Discovery plate reader.

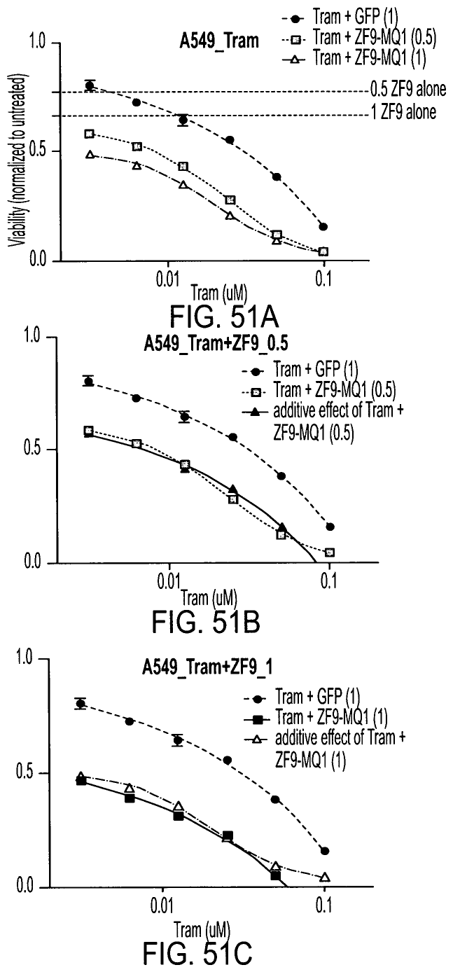

The data showed ZF9-MQ1 and Trametinib each separately inhibited cell viability of A549 cells (FIG. 51A). When combined, ZF9-MQ1 (0.5 μg/ml) and Trametinib (concentrations up to 0.05 uM) showed a greater than additive effect on the inhibition of A549 viability than what is predicted by their individual activities (FIG. 51B). When combined, ZF9-MQ1 (1.0 μg/ml) and Trametinib (concentrations up to 0.05 uM) showed a greater than additive effect on the inhibition of A549 viability (FIG. 51C) than what is predicted by their individual activities. The data suggested that ZF9-MQ1 and Trametinib combination was synergistic to inhibit viability in A549 cells (FIG. 51B-51C).

### Example 52: Study on MYC Downregulation with Panel of Super Enhancer Region Targeting ZF-KRAB Constructs in Multiple Cell Lines

This example evaluates the effect of ZF9-MQ1 and a panel of super-enhancer region targeting zinc finger constructs ZF9-MQ1, ZF54-KRAB, ZF61-KRAB, ZF67-KRAB, and ZF68-KRAB on MYC downregulation in H2009, H460, and H226 cells.

H2009 and H226 cells were seeded at 5,000 cells per well in duplicate plates for mRNA analysis. The 96-well plates were treated in triplicate with GFP, ZF9-MQ1, ZF54-KRAB, ZF67-KRAB, or ZF68-KRAB loaded LNPs at 1.0 μg/mL and incubated. H226 cells were also treated in triplicate with non-coding RNA loaded LNPs at 1.0 μg/mL and incubated. H460 cells were seeded at 5,000 cells per well in duplicate plates for mRNA analysis. The 96-well plates were treated in triplicate with GFP, ZF9-MQ1, ZF61-KRAB, ZF67-KRAB, or ZF68-KRAB loaded LNPs at 1.0 μg/mL an incubated. Untreated cells and GFP treated cells were used as controls. After incubating for 72 hours, the cells were lysed with RLT Plus Lysis buffer for mRNA extraction using Rneasy® Plus 96 Kit. The lysed sample was bound to an RNA column, washed with buffers, and eluted off mRNA was then converted to cDNA using poly-A primers with RT Lunascript. cDNA was then used for reverse transcription polymerase chain reaction (RT-PCR) analysis using TaqMan™ probes (Thermo Fisher) specific for human MYC mRNA transcripts. GAPDH mRNA transcript levels were used for normalization across groups.

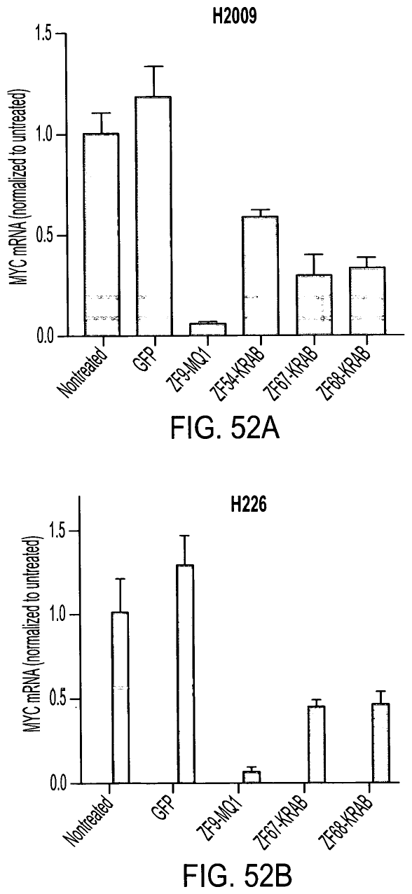

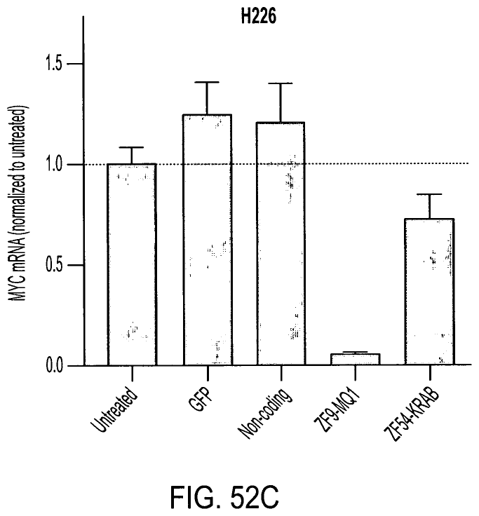

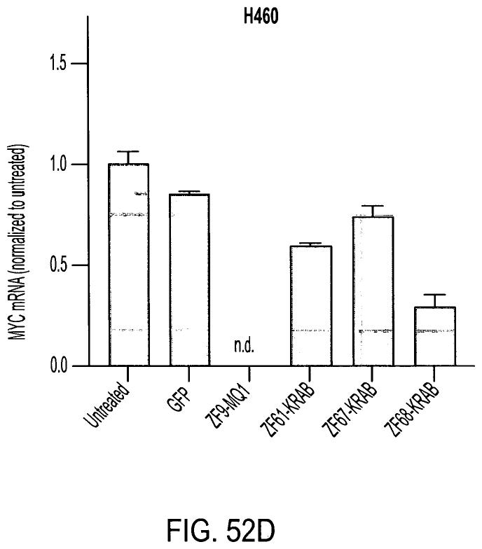

The data showed that ZF9-MQ1, ZF54-KRAB, ZF67-KRAB, and ZF68-KRAB downregulated MYC mRNA levels in H2009 by at least 42% (FIG. 52A) and H226 cells by at least 27% in comparison to untreated cells (FIG. 52B-52C). In addition, ZF9-MQ1, ZF61-KRAB, ZF67-KRAB, and ZF68-KRABdownregulated MYC mRNA levels in H460 cells by at least 26% compared to untreated cells (FIG. 52D).

### Example 53: Co-Treatment of ZF54-KRAB and ZF9-MQ1 Decrease MYC mRNA Level Further Compared to ZF9-MQ1 Alone

This example evaluates the effect of combined treatment of ZF9-MQ1 and ZF54-KRAB on MYC downregulation in H2009 cells.

H2009 cells were seeded at 5,000 cells per well in a 96 well plate for mRNA analysis. The walls in the plate was treated in triplicate with GFP, ZF9-MQ1, loaded LNPs at 1.0 μg/mL or 2.0 μg/mL combination with increasing concentrations of ZF9-MQ1-loaded LNPs and incubated for 72 hours. Untreated cells and GFP treated cells were used as controls. After incubation, cells were lysed with RLT Plus Lysis buffer for mRNA extraction using Rneasy® Plus 96 Kit. The lysed sample was bound to an RNA column, washed with buffers, and eluted off mRNA was then converted to cDNA using poly-A primer with RT Lunascript. cDNA was then used for reverse transcription polymerase chain reaction (RT-PCR) analysis using TaqMan™ probes (Thermo Fisher) specific for human MYC mRNA transcripts. GAPDH mRNA transcript levels were used for normalization across groups.

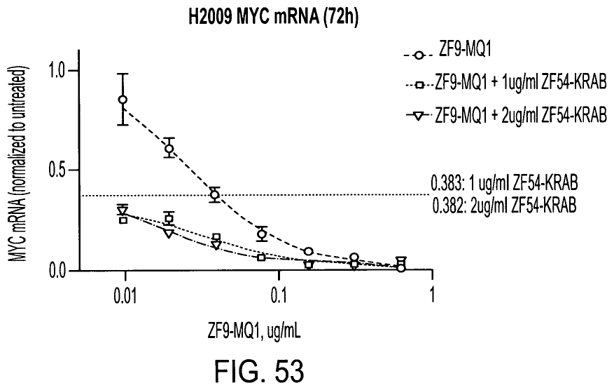

The data showed that at the highest concentration tested, ZF9-MQ1 and ZF54-KRAB each separately downregulated MYC mRNA in H2009 cells by 99% or 62% respectively, relative to untreated control cells. When less than 0.313 μg/mL ZF9-MQ1 is combined with 1 or 2 μg/mL ZF54-KRAB, MYC mRNA is downregulated to a greater extent than that observed for either treatment alone indicating that each ZF9-MQ1 and ZF54-KRAB contributed to downregulation of MYC mRNA levels in H2009 cells line in vitro (FIG. 53).

### Example 54: The 1:1 Combination of ZF9-MQ1 and ZF54-KRAB Suppressed MYC Expression for all Time Points for Up to at Least 6 Days

This example evaluates the durability of the combined treatment of ZF9-MQ1 and ZF54-KRAB on MYC downregulation in H1299 cells. H1299 cells were seeded at 10,000 cells per well in 96 well plates for mRNA analysis. The walls in the 96-well plate was treated in triplicate with GFP, ZF54-KRAB, or ZF9-MQ1+ZF54-KRAB loaded LNPs at 1.0 μg/mL and incubated. Untreated cells and GFP treated cells were used as controls. Measurements were made at 6 hour, 1 day, 2 days, 3 days, or 6 days post transfection. After incubation, one 96-well plate was lysed with RLT Plus Lysis buffer for mRNA extraction using Rneasy® Plus 96 Kit. The lysed sample was bound to an RNA column, washed with buffers, and eluted off. mRNA was then converted to cDNA using poly-A primer with RT Lunascript. cDNA was then used for reverse transcription polymerase chain reaction (RT-PCR) analysis using TaqMan™ probes (Thermo Fisher) specific for human MYC mRNA transcripts. GAPDH mRNA transcript levels were used for normalization across groups.

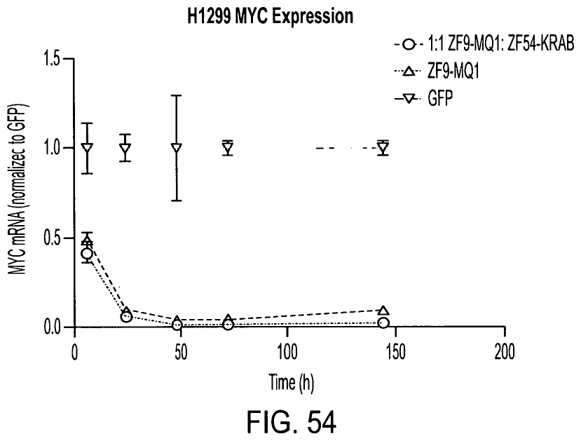

The data showed that ZF9-MQ1 downregulated MYC mRNA in H1299 cells by 95% relative to untreated control cells by 48 hours and maintained downregulation at 90% of control levels at 144 hours. Combination of ZF9-MQ1 plus ZF54-KRAB reduced MYC mRNA levels to 98% at 48 hours and maintained downregulation to 93% of control levels at 144 hours (FIG. 54). Further, the data showed ZF9-MQ1 and ZF9-MQ1 combined with ZF54-KRAB downregulated MYC mRNA levels in H1299 cells for at least 6 days (FIG. 54).

### Example 55: Bi-Cistronic ZF9-MQ1_ ZF54-KRAB Inhibits MYC Levels Earlier than ZF9-MQ1

This example evaluates the efficacy of bi-cistronic constructs ZF9-MQ1_ZF54-KRAB and ZF54-KRAB_ZF9-MQ1 compared to ZF9-MQ1 and ZF54-KRAB construct alone.

H2009 cells were seeded at 5,000 cells per well in a 96 well plate for mRNA analysis. The walls of the 96 well plate were treated in triplicate with GFP, ZF9-MQ1, ZF54-KRAB, ZF9-MQ1_ZF54-KRAB or ZF54-KRAB_ZF9-MQ1 loaded LNPs at 0.5 μg/mL or 1.0 μg/mL and incubated. Untreated cells and GFP treated cells were used as controls. Measurements were made at 24 hours and 48 hours post transfection. After incubation, one 96-well plate was lysed with RLT Plus Lysis buffer for mRNA extraction using Rneasy® Plus 96 Kit. The lysed sample was bound to an RNA column, washed with buffers, and eluted off. mRNA was then converted to cDNA using poly-A primer with RT Lunascript. cDNA was then used for reverse transcription polymerase chain reaction (RT-PCR) analysis using TaqMan™ probes (Thermo Fisher) specific for human MYC mRNA transcripts. GAPDH mRNA transcript levels were used for normalization across groups.

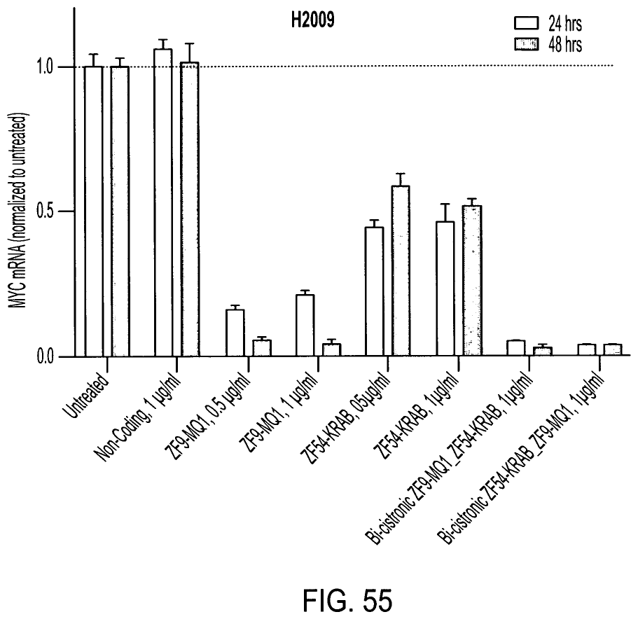

The data showed that 24 hours after introduction to H2009 cells, ZF9-MQ1 and ZF54-KRAB downregulated MYC mRNA levels by up to 83% and 55%, respectively, in comparison to untreated cells. MYC mRNA levels were further reduced by another 13% in ZF9-MQ1-treated cells to 96% of untreated controls 48 hours after treatment, whereas ZF54-KRAB did not further downregulate MYC levels. MYC mRNA levels in cells treated with ZF9-MQ1_ZF54-KRAB and ZF54-KRAB_ZF9-MQ1 were reduced to 95% and 96% of control cells, respectively, at 24 hours post-treatment. The data indicated that these controllers were able to reduce MYC mRNA levels earlier than ZF9-MQ1 leading to a greater level of MYC downregulation in treated cells compared ZF9-MQ1 treated cells at 24 hours in H2009 cells. (FIG. 55).

### Example 56: Effect of ZF9-MQ1 Treatment on Inhibiting Tumor Growth in Mice Bearing H460 SQ Tumor

This example demonstrates that ZF9-MQ1 inhibits the growth of subcutaneous H460 tumors established in nude mice.

Disease was induced in nude mice by the implantation of subcutaneous H460 tumor cells into the left flank. Treatment was initiated when mean tumor volume reached approximately 100-150 mm³. Mice were divided into treatment groups so that mean tumor volume in each group were approximately equal. Tumors were treated with PBS (vehicle), sorafenib (standard of care), non-coding RNA formulated in MC3 LNPs, or ZF9-MQ1 mRNA formulated in MC3 LNPs. PBS, Non-coding RNA (SEQ ID NO: 198) LNP and ZF9-MQ1 LNP were dosed at 3 mg/kg administered every five days. Sorafenib was dosed daily at 50 mg/kg. The length and width of tumors were measured twice weekly. Tumor volumes were calculated as (width²×length)/2.

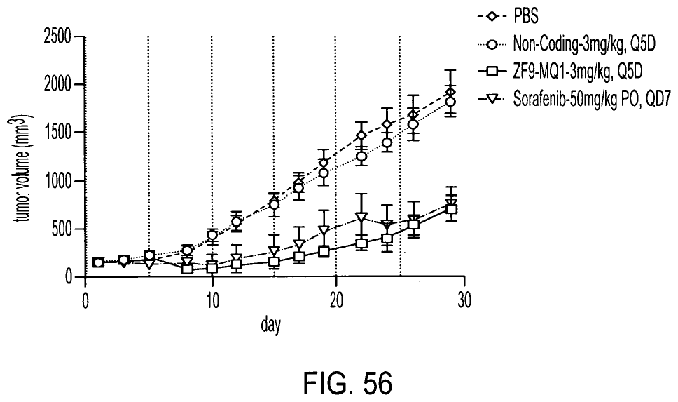

The results showed that ZF9-MQ1 treatment inhibited tumor growth in the H460 subcutaneous tumor model. The average tumor volumes at the study termination were 1921 mm³, 1829 mm³, and 702 mm³ in PBS-treated, mRNA negative control-treated, and ZF09-MQ1-treated groups, respectively. Average tumor volumes in sorafenib-treated mice (752 mm³) were indistinguishable from ZF9-MQ1-LNP treated mice, indicating that ZF9-MQ1 exhibited a similar or better activity standard of care agent sorafenib in this model. These results demonstrated that in comparison to the negative control groups, ZF9-MQ1-loaded LNPs were effective in inhibiting tumor growth in the in vivo H460 subcutaneous tumor model (FIG. 56).

### EQUIVALENTS

It is to be understood that while the invention has been described in conjunction with the detailed description thereof, the foregoing description is intended to illustrate and not limit the scope of the invention, which is defined by the scope of the appended claims. Some aspects, advantages, and modifications are within the scope of the following claims.

We claim:

1. An expression repressor comprising:
a first targeting moiety that binds a genomic locus comprising at least 16, 17, 18, 19, or 20 nucleotides of the sequence of SEQ ID NO: 83, and
a first effector moiety comprising a DNA methyltransferase.

2. The expression repressor of claim 1, wherein the first targeting moiety comprises a zinc finger domain.

3. The expression repressor of claim 1 or 2, wherein the first targeting moiety comprises an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

4. The expression repressor of any of the preceding claims, wherein the first effector moiety comprises MQ1 or a functional variant or fragment thereof.

5. The expression repressor of any of the preceding claims, wherein the first effector moiety comprises a sequence of SEQ ID NO: 19 or 87, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

6. The expression repressor of any of the preceding claims, wherein the first effector moiety comprises a sequence of SEQ ID NO: 30 or 129, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

7. A nucleic acid encoding the expression repressor of any of the preceding claims.

8. The nucleic acid of claim 7, which comprises a nucleotide sequence encoding the first targeting moiety, wherein the nucleotide sequence encoding the first targeting moiety comprises a sequence according to SEQ ID NO: 46 or 131 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

9. The nucleic acid of claim 7 or 8, which comprises a nucleotide sequence encoding the first effector moiety, wherein the nucleotide sequence encoding the first effector moiety comprises a sequence according to SEQ ID NO: 52 or 132, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

10. The nucleic acid of any of claims 7-9, which comprises a nucleotide sequence according to SEQ ID NO: 63 or 130, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.

11. A system comprising:
a first expression repressor according to any of claims 1-6, and
a second expression repressor.

12. The system of claim 11, wherein the second expression repressor comprises:
a second targeting moiety that binds a genomic locus, and
a second effector moiety.

13. The system of claim 12, wherein the second targeting moiety binds a genomic locus comprising at least 14, 15, 16, 17, 18, 19, or 20 nucleotides of the sequence of SEQ ID NO: 77 199 or 201.

14. The system of claim 12 or 13, wherein the second targeting moiety comprises a zinc finger domain.

15. The system of any of claims 12-14, wherein the second targeting moiety comprises an amino acid sequence according to SEQ ID NO: 7 169, 171, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

16. The system of any of claims 12-15, wherein the second effector moiety comprises KRAB or a functional variant or fragment thereof.

17. The system of any of claims 12-16, wherein the second effector moiety comprises an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

18. The system of any of claims 12-17, wherein the second expression repressor comprises an amino acid sequence according to SEQ ID NO: 24 177, 183, 179, or 185 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

19. A nucleic acid encoding the first expression repressor and second repressor of the system of any of claims 12-18.

20. The nucleic acid of claim 19, which comprises a nucleotide sequence according to SEQ ID NO: 113, 196, or 197, wherein a poly-A sequence is optional.

21. An expression repression system comprising:
a) a first expression repressor comprising:
i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto and
ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second expression repressor comprising:
i) a second targeting moiety having an amino acid sequence according to SEQ ID NO: 7 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
ii) a second effector moiety having an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

22. An expression repression system comprising:
a) a first expression repressor comprising:
i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto and
ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second expression repressor comprising:
i) a second targeting moiety having an amino acid sequence according to SEQ ID NO: 169, 171 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
ii) a second effector moiety having an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

23. An expression repression system comprising:
a) a first expression repressor comprising:
i) a first targeting moiety having an amino acid sequence according to SEQ ID NO: 169, 171, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto and
ii) a first effector moiety having an amino acid sequence according to SEQ ID NO: 18 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second expression repressor comprising:
i) a second targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
ii) a second effector moiety having an amino acid sequence according to SEQ ID NO: 19 or 87, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

24. The expression repressor or expression repression system of any of the preceding claims, which appreciably decreases expression of MYC for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA.

25. A fusion protein comprising a first amino acid region encoding a first expression repressor described herein and a second amino acid region comprising a second expression repressor described herein.

26. The expression repressor, fusion protein, or expression repression system of any of the preceding claims, wherein binding of the expression repressor to the MYC locus appreciably decreases expression of MYC at 1, 2, 3, 4, 5, 6, 7, 8, 10, 12, 16, 20, 24, 28, 32, 36, 40, 44, 48, 52, 56, 60, 64, 68, 72, 76, 80, or 96 hours post-transfection with the expression repressor or expression repression system.

27. The expression repressor, fusion protein, or expression repression system of any of the preceding claims, wherein contacting a plurality of cells with the expression repressor, expression repressor system, or a nucleic acid encoding the expression repressor or the first expression repressor and the second expression repressor decreases the viability of the plurality of cells.

28. The expression repressor, fusion protein, or expression repression system of any of the preceding claims, wherein, administration of the expression repressor or expression repression system result in apoptosis of at least 5%, 6%, 7%, 8%, 9% 10%, 12%, 15%, 17% 20%, 25% 30%, 40%, 45%, 50%, 55%, 60%, 65%, 75% of target cells (e.g., cancer cells).

29. The expression repressor, fusion protein, or expression repression system of claim 17d, wherein the plurality of cells comprises a plurality of cancer cells and a plurality of non-cancer cells.

30. The expression repressor, fusion protein, or expression repression system of claim 17e, wherein contacting the plurality of cells with the system or a nucleic acid encoding the system decreases the viability of the plurality of cancer cells more than it decreases the viability of the plurality of non-cancer cells, optionally wherein the viability of the plurality of cancer cells decreases 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than the viability of the plurality of non-cancer cells.

31. A nucleic acid comprising a sequence encoding the expression repressor, fusion protein, or system of any of claims 1-30.

32. A nucleic acid encoding an expression repression system, the nucleic acid comprising:
a) a first region encoding a first expression repressor, the first expression repressor comprising:
i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second region encoding a second expression repressor, the second expression repressor comprising:
i) a second targeting moiety having an amino acid sequence according to SEQ ID NO: 7 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
ii) a second effector moiety having an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

33. A nucleic acid encoding an expression repression system, the nucleic acid comprising:
a) a first region encoding a first expression repressor, the first expression repressor comprising:
i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second region encoding a second expression repressor, the second expression repressor comprising:
i) a second targeting moiety having an amino acid sequence according to SEQ ID NO: 169, 171, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
ii) a second effector moiety having an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

34. A nucleic acid encoding an expression repression system, the nucleic acid comprising:
a) a first region encoding a first expression repressor, the first expression repressor comprising:
i) a first targeting moiety having an amino acid sequence according to SEQ ID NO: 169, 171, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
ii) a first effector moiety having an amino acid sequence according to SEQ ID NO: 18 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
b) a second region encoding a second expression repressor, the second expression repressor comprising:
i) a second targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
ii) a second effector moiety having an amino acid sequence according to SEQ ID NO:19 or 87, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

35. The nucleic acid of any of claim 19, 21 or 22, which comprises a nucleotide sequence of SEQ ID NO: 93, 94, 112, 113, 196, 197, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.

36. An expression repressor comprising:
a targeting moiety that binds a genomic locus comprising at least 16, 17, 18, 19, or 20 nucleotides of a sequence of Table 12, and
optionally, an effector moiety.

37. An expression repressor comprising:
a targeting moiety having an amino acid sequence according to Table 4, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
optionally, an effector moiety.

38. The expression repressor of claim 37, which comprises an amino acid sequence according to Table 7 or Table 9, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

39. A nucleic acid comprising a sequence according to Table 5, Table 6, Table 8, Table 16, or Table 10, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

40. A vector comprising the nucleic acid encoding the fusion protein, system, or expression repressor of any of the preceding claims.

41. A reaction mixture comprising the expression repressor, system, fusion protein, nucleic acid, or vector of any of the preceding claims.

42. A pharmaceutical composition comprising the expression repressor, system, fusion protein, nucleic acid, vector, or reaction mixture of any of the preceding claims.

43. A method of treating cancer in a subject in need thereof, the method comprising:
administering the expression repressor, the system, the fusion protein, or the nucleic acid encoding the expression repressor or the system of any of claims 1-20.

44. The method of claim 43, wherein the cancer is a hepatocellular carcinoma (HCC), Fibrolamellar Hepatocellular Carcinoma (FHCC), Cholangiocarcinoma, Angiosarcoma, secondary liver cancer, lung cancer, Non-small cell lung cancer (NSCLC), Adenocarcinoma, Small cell lung cancer (SCLC), Large cell (undifferentiated) carcinoma, triple negative breast cancer, gastric adenocarcinoma, endometrial carcinoma, or pancreatic carcinoma.

45. A method of treating hepatitis in a subject in need thereof, the method comprising:
administering the expression repressor, the fusion protein, the system, or the nucleic acid encoding the expression repressor or the system of any of claims 1-42.

46. A method of reducing tumor growth in a subject in need thereof, the method comprising:
administering the expression repressor, fusion protein, system, nucleic acid, vector, or a pharmaceutical composition of any of claims 1-42 to the subject,
thereby reducing the tumor growth in the subject.

47. A method of increasing or restoring sensitivity of a cancer to a kinase inhibitor, e.g., sorafenib, the method comprising administering an expression repressor or system of any of claims 1-42 to a subject having the cancer, optionally wherein administration of the expression repressor or system lowers the IC₅₀ of the kinase inhibitor by 10%, 20%, 30%, or 40%, e.g., in a cancer cell viability assay.

48. A method of increasing or restoring sensitivity of a cancer to a bromodomain inhibitor, e.g., a BET inhibitor, e.g., JQ1, the method comprising administering an expression repressor or system of any of t claims 1-42 to a subject having the cancer, wherein optionally administration of the expression repressor or system lowers the IC₅₀ of the bromodomain inhibitor by 10%, 20%, 30%, 40%, 50%, 60%, 70%, 80%, 90%, or 95%, e.g., in a cancer cell viability assay.

49. A method of increasing or restoring sensitivity of a cancer to a MEK inhibitor, e.g., Trametinib, the method comprising administering an expression repressor or system any of claims 1-42 to a subject having the cancer, wherein optionally administration of the expression repressor or system lowers the IC₅₀ of the MEK inhibitor by 10%, 20%, 30%, 40%, 50%, 60%, 70%, 80%, 90%, or 95%, e.g., in a cancer cell viability assay.

50. The method of any of claims 43-49, wherein the cancer comprises cells characterized by increased MYC expression relative to a reference level (e.g., relative to a reference cell's MYC expression, e.g., an otherwise similar non-cancerous cell of the subject), and cells not characterized by increased MYC expression relative to a reference level (e.g., relative to a reference cell's MYC expression, e.g., an otherwise similar non-cancerous cell of the subject), e.g., having normal MYC expression.

51. The method of any of claims 43-50, comprising:
a) first, administering to the subject a first plurality of doses of an expression repressor or system of any of claims_wherein optionally each subsequent dose in the first plurality is administered 5 days after the previous dose in the first plurality;
b) second, withdrawing the expression repressor or system for a period of time (a “drug holiday”), e.g., for about 2 weeks), and
c) third, administering to the subject a second plurality of doses of the expression repressor or system, wherein optionally a subsequent dose of the second plurality is administered 5 days after the previous dose in the second plurality.

52. The method of any of claims 43-51, wherein tumor volume declines (e.g., to undetectable levels) after cessation of treatment with the expression repressor, fusion protein, or system.

53. The method of any of claims 43-52, wherein the method further comprises
a. contacting the cell with a second therapeutic agent or
b. administering a second therapeutic agent to the subject, optionally wherein the second therapeutic agent is not an expression repressor, system, fusion protein, nucleic acid, vector, reaction mixture, or pharmaceutical composition, of any of claims_.

54. The method of any of claims 43-53, wherein the second therapeutic agent is an immunotherapy, one or both of immune checkpoint and anti-vascular-endothelial-growth-factor therapy, systemic chemotherapy, a tyrosine kinase inhibitor, e.g., sorafenib, a mitogen-activated protein kinase kinase inhibitor, e.g., trametinib, or a bromodomain inhibitor, e.g., a BET inhibitor, e.g., JQ1, e.g., birabresib.

55. The method of any of claims 43-54, wherein the first and the second therapeutic agent are administered concurrently.

56. The method of any of claims 43-55, wherein the first and the second therapeutic agent are administered sequentially.

57. The method of any of claims 43-56, wherein the subject has an overexpression of MYC in at least some cells.
